(19) United States
(12) Patent Application Publication
(10) Pub. No.: US 2007/0142369 A1
(43) Pub. Date: Jun. 21, 2007

(54) Combination of an H3 antagonist/inverse agonist and an appetite suppressant

(76) Inventors: Margaret van Heek, Scotch Plains, NJ (US); Joyce J. Hwa, Westfield, NJ (US); Michael P. Graziano, Scotch Plains, NJ (US); Jean E. Lachowicz, Berkeley Heights, NJ (US); Timothy J. Kowalski, Plainsboro, NJ (US); Enrico P. Veltri, Princeton, NJ (US); Kevin D. McCormick, Basking Ridge, NJ (US); Michael Y. Berlin, Flemington, NJ (US); Robert G. Aslanian, Rockaway, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/640,729

(22) Filed: Dec. 18, 2006

Related U.S. Application Data
(60) Provisional application No. 60/752,323, filed on Dec. 21, 2005.

(51) Int. Cl. A61K 31/541; A61K 31/517; A61K 31/4745; A61K 31/4192; A61K 31/428; A61K 31/496; A61K 31/5377; A61K 31/423; A61K 31/4184; A61K 31/4178

(52) U.S. Cl. 514/227.5; 514/373; 514/397; 514/394; 514/303; 514/266.2; 514/367; 514/359; 514/375; 514/227.8; 514/235.5; 514/254.05

(57) ABSTRACT

The present invention relates to pharmaceutical compositions comprising therapeutic combinations comprising: one or more H₃ antagonists/inverse agonists; one or more appetite suppressants selected from the group consisting of CB₁ antagonists/inverse agonists, sibutramine, phentermine and topiramate; and optionally one or more HMG-CoA reductase inhibitors. The invention also relates to medicaments and kits comprising the pharmaceutical compositions of the present invention, and methods of treating obesity, obesity related disorders and diabetes using the pharmaceutical compositions of the present invention.

This application claims the benefit of U.S. Provisional Application No. 60/752,323, filed Dec. 21, 2005, which is incorporated by reference herein in its entirety.

### FIELD OF THE INVENTION

The present invention relates to pharmaceutical compositions comprising therapeutic combinations comprising: one or more H₃ antagonists/inverse agonists; one or more appetite suppressants selected from the group consisting of CB₁ antagonists/inverse agonists, sibutramine, phentermine and topiramate; and optionally one or more HMG-CoA reductase inhibitors. The invention also relates to medicaments and kits comprising the pharmaceutical compositions of the present invention, and methods of treating obesity, obesity related disorders and diabetes using the pharmaceutical compositions of the present invention.

### BACKGROUND OF THE INVENTION

The histamine receptors, H₁, H₂, H₃ and H₄ have been characterized by their pharmacological behavior. The H₁ receptors are those that mediate the response antagonized by conventional antihistamines. H₁ receptors are present, for example, in the ileum, the skin, and the bronchial smooth muscle of humans and other mammals. The most prominent H₂ receptor-mediated responses are the secretion of gastric acid in mammals and the chronotropic effect in isolated mammalian atria. H₄ receptors are expressed primarily on eosinophils and mast cells and have been shown to be involved in the chemotaxis of both cell types.

In the periphery, H₃ receptor sites are found on sympathetic nerves, where they modulate sympathetic neurotransmission and attenuate a variety of end organ responses under control of the sympathetic nervous system. Specifically, H₃ receptor activation by histamine attenuates norepinephrine outflow to resistance and capacitance vessels, causing vasodilation. In addition, in rodents, peripheral H₃ receptors are expressed in brown adipose tissue, suggesting that they may be involved in thermogenesis regulation.

H₃ receptors are also present in the CNS. H₃ receptor expression is observed in cerebral cortex, hippocampal formation, hypothalamus and other parts of the human and animal brain. H₃ receptors are expressed on histaminergic neurons where they function as autoreceptors and, on neurons involved in other neurotransmitter systems, where they function as heteroreceptors. In both cases H₃ receptor activation results in presynaptic inhibition of neurotransmitter release. In the particular case of histaminergic neurons, H₃ receptors have been implicated in the regulation of hypothalamic histamine tone, which in turn has been associated with the modulation of sleeping, feeding and cognitive processes in the human brain (see, for example, Leurs et al., Nature Reviews, Drug Discovery, 4, (2005), 107).

It is also known and has been described in the literature that histamine is involved in regulation of cognitive and memory processes in the human brain (see, for example, Life Sciences, 72, (2002), 409-414). Consequently, indirect modulation of histaminergic brain function through the central H₃ receptors may be a means to modulate these processes. Different classes of H₃ receptor ligands have been described and their use for neurological and psychiatric diseases has been suggested (see, e.g., US Patent Publication No. 20040224953, International Publication No. WO2004089373, International Publication No. WO2004101546). H₃ receptor antagonists may be useful in treating various neuropsychiatric conditions, where cognitive deficits are an integral part of the disease, specifically ADHD, schizophrenia and Alzheimer's disease.

Imidazole H₃ receptor antagonists are well known in the art. More recently, non-imidazole H₃ receptor antagonists have been disclosed in U.S. Pat. Nos. 6,720,328 and 6,849,621, and in US Published Applications 2004/0097483, 2004/0048843 and 2004/0019099.

U.S. Pat. No. 5,869,479 discloses compositions for the treatment of the symptoms of allergic rhinitis using a combination of at least one histamine H₁ receptor antagonist and at least one histamine H₃ receptor antagonist.

WO 95/14007 discloses H₃ receptor antagonists of the imidazole type.

WO 99/24405 discloses H₃ receptor ligands of the imidazole type.

U.S. Pat. No. 5,869,479 discloses compositions for the treatment of the symptoms of allergic rhinitis using a combination of at least one histamine H₁ receptor antagonist and at least one histamine H₃ receptor antagonist.

HMG-CoA reductase inhibitors, e.g., statins such as lovastatin, simvastatin, pravastatin, atorvastatin, fluvastatin, and resuvastatin, slow the progression of atherosclerotic lesions in the coronary and carotid arteries. Simvastatin, atorvastatin and pravastatin have also been shown to reduce the risk of coronary heart disease events in patients with hypercholesterolemia and/or atherosclerotic coronary heart disease (CHD).

Simvastatin is marketed worldwide, and sold in the U.S. under the tradename ZOCOR®. Methods for making it are described in U.S. Pat. Nos. 4,444,784; 4,916,239; 4,820,850; among other patent and literature publications.

The CB₁ receptor is one of the most abundant neuromodulatory receptors in the brain, and is expressed at high levels in the hippocampus, cortex, cerebellum, and basal ganglia (e.g., Wilson et al., Science, 2002, vol. 296, 678-682). Selective CB₁ receptor antagonists, for example pyrazole derivatives such as rimonabant, can be used to treat various conditions, such as obesity and metabolic syndrome (e.g., Bensaid et al., Molecular Pharmacology, 2003 vol. 63, no. 4, pp. 908-914; Trillou et al., Am. J. Physiol. Regul. Integr. Comp. Physiol. 2002 vol. 284, R345-R353; Kirkham, Am. J. Physiol. Regul. Integr. Comp. Physiol. 2002 vol. 284, R343-R344; Sanofi-Aventis Publication, Bear Stearns Conference, New York, Sep. 14, 2004; Nicole Cranois and Jean-Marc Podvin, Sanofi-Synthelabo, press release reporting results of RIO-LIPIDS AND STRATUS-US Study results, American College of Cardiology Annual Meeting, New Orleans, Mar. 9, 2004;), neuroinflammatory disorders (e.g., Adam, et al., Expert Opin. Ther. Patents, 2002, vol.12, no. 10, 1475-1489), cognitive disorders, psychosis, addiction, gastrointestinal disorders (e.g., Lange et al., J. Med. Chem. 2004, vol. 47, 627-643) and cardiovascular conditions (e.g., Porter et al., Pharmacology and Therapeutics, 2001 vol. 90, 45-60).

Recently, it has been shown that treatment of subjects with CB₁ receptor antagonists (e.g., rimonabant) can increase serum high density lipoprotein cholesterol (HDL-C) levels, decrease triglyceride levels and decrease waist circumference in patients (Sanofi-Aventis Publication, Bear Stearns Conference, New York, Sep. 14, 2004, pages 19-24).

Sibutramine has been shown to reduce food intake (e.g., Halford et al., British Journal of Pharmacology 1994, 114: Proc Suppl (387P); Stricker-Krongrad et al., International Journal of Obesity 1995, 19: Suppl 2 (145)) and increase oxygen consumption and body core temperature (Connoley et al., British Journal of Pharmacology 1994, 114:Proc Suppl (388P)).

Phentermine is an appetite suppressant used for treating obesity (e.g., D. Craddock, Drugs 1976; 11:378).

WO 2004/110368 describes combination therapies for the treatment of hypertension comprising the combination of an anti-obesity agent and an anti-hypertensive agent.

WO 2005/000217 describes combination therapies for the treatment of dyslipidemia comprising the administration of a combination of an anti-obesity agent and an anti-dyslipidemic agent.

WO 2004/110375 describes combination therapies for the treatment of diabetes comprising the administration of a combination of an anti-obesity agent and an anti-diabetic agent.

US 2004/0122033 describes combination therapies for the treatment of obesity comprising the administration of a combination of an appetite suppressant and/or metabolic rate enhancers and/or nutrient absorption inhibitors. US 2004/0229844 describes combination therapies for treating atherosclerosis comprising the administration of a combination of nicotinic acid or another nicotinic acid receptor agonist and a DP receptor antagonist.

However, none of the above patents, published patent applications or articles expressly describes the combination of an H₃ antagonist/inverse agonist with an appetite suppressant selected from the group consisting of a CB₁ antagonist (e.g., rimonabant), sibutramine, phentermine and topiramate

U.S. Pat. Nos. 6,437,147, 6,756,384, and 2003/0135056 describe combinations of imidazo heterocyclic compounds which bind to the H₃ receptor with antiobesity agents or appetite regulating agents, including sibutramine, phentermine, topiramate, lovastatin, pravastatin, and simvastatin. However, the compounds of U.S. Pat. Nos. 6,437,147, 6,756,384, and 2003/0135056 which bind to the H₃ receptor are different from the H₃ antagonists/inverse agonists of Formulae (I)-(VI) of the present invention.

U.S. Pat. No. 6,673,829 and 2003/0130253 describe combinations of aminoazetidine, pyrrolidine, and piperidine derivatives which bind to the H₃ receptor with antiobesity agents or appetite regulating agents, including sibutramine, phentermine, topiramate, lovastatin, pravastatin, and simvastatin. However, the compounds of U.S. Pat. No. 6,673,829 and 2003/0130253 which bind to the H₃ receptor are different from the H₃ antagonists/inverse agonists of Formulae (I)-(VI) of the present invention.

U.S. Pat. No. 6,417,218 and 2002/0058659 describe combinations of imidazole compounds which bind to the H₃ receptor with antiobesity agents or appetite regulating agents, including sibutramine, phentermine, topiramate, lovastatin, pravastatin, and simvastatin. However, the compounds of U.S. Pat. No. 6,417,218 and 2002/0058659 which bind to the H₃ receptor are different from the H₃ antagonists/inverse agonists of Formulae (I)-(VI) of the present invention.

U.S. 2004/0248938 and 2003/0186963 describe combinations of substituted piperidines which bind to the H₃ receptor with antiobesity agents or appetite regulating agents, including sibutramine, phentermine, topiramate, lovastatin, pravastatin, and simvastatin. However, the compounds of U.S. 2004/0248938 and 2003/0186963 which bind to the H₃ receptor are different from the H₃ antagonists/inverse agonists of Formulae (I)-(VI) of the present invention.

### SUMMARY OF THE INVENTION

In one embodiment, the present invention is directed to a composition comprising one or more appetite suppressants selected from the group consisting of CB₁ antagonists/inverse agonists (e.g., rimonabant), sibutramine, phentermine, and topiramate, in combination with one or more metabolic rate enhancers comprising an H₃ antagonist/inverse agonist of Formula (I)-(VII) (as defined herein).

In another embodiment, the present invention is directed to a pharmaceutical composition comprising one or more appetite suppressants selected from the group consisting of CB₁ antagonists/inverse agonists (e.g., rimonabant), sibutramine, phentermine, and topiramate, in combination with one or more metabolic rate enhancers comprising an H₃ antagonist/inverse agonist of Formula (I)-(VIII) (as defined herein), and at least one pharmaceutically acceptable carrier.

In another embodiment, the present invention is directed to a pharmaceutical composition comprising one or more appetite suppressants selected from the group consisting of CB₁ antagonists/inverse agonists (e.g., rimonabant), sibutramine, phentermine, and topiramate, in combination with one or more metabolic rate enhancers comprising an H₃ antagonist/inverse agonist, and one or more HMG-CoA reductase inhibitors.

In another embodiment, the present invention is directed to a method of treating obesity or an obesity-related disorder. The method comprises administering to the patient an effective amount of a composition comprising one or more appetite suppressants selected from the group consisting of CB₁ antagonists/inverse agonists (e.g., rimonabant), sibutramine, phentermine, and topiramate, in combination with one or more metabolic rate enhancers comprising an H₃ antagonist/inverse agonist of Formula (I)-(VIII) (as defined herein).

In another embodiment, the present invention is directed to a method of treating obesity or an obesity-related disorder. The method comprises administering to the patient an effective amount of one or more appetite suppressants selected from the group consisting of CB₁ antagonists/inverse agonists (e.g., rimonabant), sibutramine, phentermine, and topiramate, in combination with one or more metabolic rate enhancers comprising an H₃ antagonist/inverse, and one or more HMG-CoA reductase inhibitors.

### DETAILED DESCRIPTION OF THE INVENTION

As used above, and throughout this disclosure, the following terms, unless otherwise indicated, shall be understood to have the following meanings:

A “patient” is a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat.

“Alkyl” means an aliphatic hydrocarbon group which may be straight or branched and comprising about 1 to about 20 carbon atoms in the chain. The alkyl groups can contain about 1 to about 12 carbon atoms in the chain, and in another embodiment, the alkyl groups can contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. The term “substituted alkyl” means that the alkyl group may be substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl.

“Alkylene” means a difunctional group obtained by removal of a hydrogen atom from an alkyl group that is defined above. Non-limiting examples of alkylene include methylene (i.e., —CH₂—), ethylene (i.e., —CH₂CH₂— or —CH(CH₃)—), propylene (i.e., —CH₂CH₂CH₂—, —CH₂CH(CH₃)—, —CH(CH₃)CH₂—, or —CH(CH₂CH₃)—), butylene (i.e., —CH₂CH₂CH₂CH₂—, —CH₂CH₂CH(CH₃)—, —CH₂CH(CH₃)CH₂—, —CH(CH₂CH₂CH₃)—, etc.). “Lower alkylene” means a group having about 1 to about 6 carbon atoms in the chain which may be straight or branched.

“Alkenyl” means a hydrocarbon group containing at least one carbon-carbon double bond and which may be straight or branched and comprising about 2 to about 15 carbon atoms in the chain. Alkenyl groups can have about 2 to about 12 carbon atoms in the chain; and in another embodiment, about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. The term “substituted alkenyl” means that the alkenyl group may be substituted by one or more substituents which may be the same or different, each substituent being independently selected from the group consisting of halo, alkyl, aryl, cycloalkyl, cyano, alkoxy, and —S(alkyl). Non-limiting examples of suitable alkenyl groups include ethenyl, propenyl (i.e., allyl), n-butenyl, 3-methylbut-2-enyl, n-pentenyl, octenyl and decenyl.

“Alkenylene” means a difunctional group obtained by removal of a hydrogen from an alkenyl group that is defined above. Non-limiting examples of alkenylene include —CH═CH—, —C(CH₃)═CH—, and —CH═CHCH₂—.

“Alkynyl” means a hydrocarbon group containing at least one carbon-carbon triple bond and which may be straight or branched and comprising about 2 to about 15 carbon atoms in the chain. Alkynyl groups can have about 2 to about 12 carbon atoms in the chain, and in another embodiment, about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. The term “substituted alkynyl” means that the alkynyl group may be substituted by one or more substituents which may be the same or different, each substituent being independently selected from the group consisting of alkyl, aryl and cycloalkyl.

“Alkynylene” means a difunctional group obtained by removal of a hydrogen from an alkynyl group that is defined above. Non-limiting examples of alkenylene include —C≡C— and —CH₂C≡—C—.

“Aryl” means an aromatic monocyclic or multicyclic ring system comprising about 6 to about 14 carbon atoms, an in another embodiment, about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, and in another embodiment, about 5 to about 10 ring atoms, in which one or more of the ring atoms is an element other than carbon, for example nitrogen, oxygen or sulfur, alone or in combination. Heteroaryls can contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidinyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl, indazolyl, and the like, in which there is at least one aromatic ring.

“Alkylene-aryl” (or aryl-alkylene-) means a group in which the aryl and alkylene are as previously described. The bond to the parent moiety is through the alkylene. The alkylene moiety can be bonded to one or more aryl moieties. Alkylene-aryls can comprise a lower alkylene group. Non-limiting examples of suitable alkylene-aryl groups include benzyl, 2-phenethyl, 2,2-diphenylethylene and naphthalenylmethyl.

“Alkylaryl” means an alkyl-aryl- group in which the alkyl and aryl are as previously described. Alkylaryls can comprise a lower alkyl group. Non-limiting examples of suitable alkylaryl groups include tolyl and xylyl. The bond to the parent moiety is through the aryl.

“Alkylheteroaryl” means an alkyl-heteroaryl- group in which the alkyl and heteroaryl are as previously described. Alkylheteroaryls can comprise a lower alkyl group. A non-limiting example of a suitable alkylheteroaryl group includes 2-methylpyridine. The bond to the parent moiety is through the heteroaryl.

“Cycloalkyl” means a non-aromatic mono- or multicyclic ring system comprising about 3 to about 10 carbon atoms, and in another embodiment, about 5 to about 10 carbon atoms. Cycloalkyl rings can contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like, as well as partially saturated species such as, for example, indanyl, tetrahydronaphthyl and the like.

“Cycloalkenyl” means an unsaturated, non-aromatic mono- or multicyclic ring system having at least 1 carbon-carbon double bond, and comprising about 3 to about 10 carbon atoms, an in another embodiment, about 5 to about 10 carbon atoms. Cycloalkenyl rings can contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopropenyl, cyclopentenyl, cyclohexenyl, cycloheptenyl and the like. Non-limiting examples of suitable multicyclic cycloalkyls include norbornenyl, adamantenyl and the like.

“Cycloalkylene” means a difunctional group obtained by removal of a hydrogen atom from a cycloalkyl group that is defined above. Non-limiting examples of cycloalkylene include

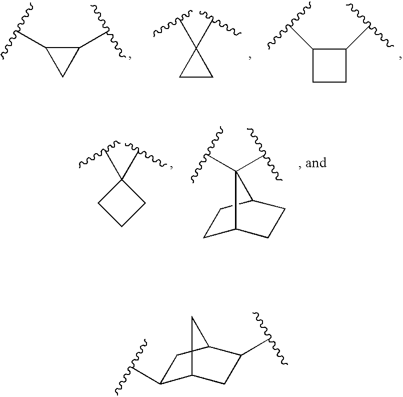

“Halogen” or “halo” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, alkylene-aryl, alkylaryl, alkylene-heteroaryl, heteroaryl-alkenylene-, heteroaryl-alkynylene-, alkylheteroaryl, hydroxy, hydroxyalkyl, alkoxy, aryloxy, aryl-alkoxy-, acyl, aroyl, halo, nitro, cyano, carboxy, alkoxycarbonyl, aryloxycarbonyl, aryl-alkoxycarbonyl, alkylsulfonyl, arylsulfonyl, heteroarylsulfonyl, alkylthio, arylthio, heteroarylthio, aryl-alkylthio, heteroaryl-alkylthio, cycloalkyl, heterocyclyl, —C(═N—CN)—NH₂, —C(═NH)—NH₂, —C(═NH)—NH(alkyl), Y¹Y²N—, Y¹Y²N-alkyl-, Y¹Y²NC(O)—, Y¹Y²NSO₂— and —SO₂NY¹Y², wherein Y¹ and Y² can be the same or different and are independently selected from the group consisting of hydrogen, alkyl, aryl, cycloalkyl, and aryl-alkylene-. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

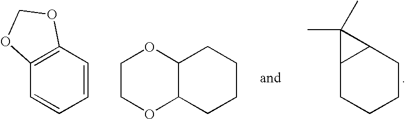

“Heterocyclyl” or “heterocyclic” means a monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Heterocyclyls may be completely saturated, partially unsaturated, or aromatic. Aromatic heterocyclyls are termed “heteroaryl”, as defined above. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBn), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include saturated heterocyclyls, for example piperidyl, pyrrolidinyl, piperazinyl, morpholinyl, thiomorpholinyl, thiazolidinyl, 1,4-dioxanyl, tetrahydrofuranyl, tetrahydrothiophenyl, lactams, lactones, and the like. Non-limiting examples of partially unsaturated monocyclic heterocyclyl rings include, for example, thiazolinyl, and the like.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring: 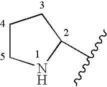
there is no —OH attached directly to carbons marked 2 and 5.

“Alkynylalkyl” means an alkynyl-alkyl- group in which the alkynyl and alkyl are as previously described. Alkynylalkyls can contain a lower alkynyl and a lower alkyl group. The bond to the parent moiety is through the alkyl. Non-limiting examples of suitable alkynylalkyl groups include propargylmethyl.

“Heteroarylalkyl” means a heteroaryl-alkyl- group in which the heteroaryl and alkyl are as previously described. Heteroaralkyls can contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Hydroxyalkyls can contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Acyls can contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aryl-alkyloxy” (or arylalkoxy) means an aryl-alkyl-O— group in which the aryl-alkyl group is as previously described. Non-limiting examples of suitable aryl-alkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aryl-alkylthio” (or arylalkylthio) means an aryl-alkyl-S— group in which the aryl-alkyl group is as previously described. Non-limiting example of a suitable aryl-alkylthio group is benzylthio. The bond to the parent moiety is through the sulfur.

“Alkoxycarbonyl” means an alkyl-O—C(O)— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Arylalkoxycarbonyl” means an aryl-alkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties. An optionally substituted moiety may be unsubstituted or substituted with one or more substituents.

The term “purified”, “in purified form” or “in isolated and purified form” for a compound refers to the physical state of said compound after being isolated from a synthetic process or natural source or combination thereof. Thus, the term “purified”, “in purified form” or “in isolated and purified form” for a compound refers to the physical state of said compound after being obtained from a purification process or processes described herein or well known to the skilled artisan, in sufficient purity to be characterizable by standard analytical techniques described herein or well known to the skilled artisan.

The term “metabolic rate enhancer” refers to compounds which improve energy expenditure.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any variable (e.g., aryl, heterocycle, R¹, etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if the appetite suppressant, metabolic rate enhancer, or HMG-CoA reductase inhibitor contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if the appetite suppressant, metabolic rate enhancer, or HMG-CoA reductase inhibitor contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If the appetite suppressant, metabolic rate enhancer, or HMG-CoA reductase inhibitor incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describes the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The term “obesity” as used herein, refers to a patient being overweight and having a body mass index (BMI) of 25 or greater. In one embodiment, an obese patient has a BMI of 25 or greater. In another embodiment, an obese patient has a BMI from 25 to 30. In another embodiment, an obese patient has a BMI greater than 30. In still another embodiment, an obese patient has a BMI greater than 40.

The term “obesity-related disorder” as used herein refers to any disorder which results from a patient having a BMI of 25 or greater. Non-limiting examples of an obesity-related disorder include edema, shortness of breath, sleep apnea, skin disorders and high blood pressure.

“Effective amount” or “therapeutically effective amount” is meant to describe an amount of compound or a composition of the present invention effective in inhibiting the diseases or conditions noted below, and thus producing the desired therapeutic, ameliorative, inhibitory or preventative effect.

The appetite suppressant, metabolic rate enhancer, or HMG-CoA reductase inhibitor of the present invention can form salts which are also within the scope of this invention. Reference to the appetite suppressant or metabolic rate enhancer of the present invention herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when the appetite suppressant, metabolic rate enhancer, or HMG-CoA reductase inhibitor of the present invention contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the appetite suppressant, metabolic rate enhancer, or HMG-CoA reductase inhibitor of the present invention may be formed, for example, by reacting the appetite suppressant, metabolic rate enhancer, HMG-CoA reductase inhibitor of the present invention with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the appetite suppressant, metabolic rate enhancer, or HMG-CoA reductase inhibitor of the present invention include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, (C₁-C₄)alkyl, or (C₁-C₄)alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a (C₁-C₂₀) alcohol or reactive derivative thereof, or by a 2,3-di-(C₆-C₂₄)acyl glycerol.

The appetite suppressant, metabolic rate enhancer, or HMG-CoA reductase inhibitor of the present invention may contain asymmetric or chiral centers, and, therefore, exist in different stereoisomeric forms. It is intended that all stereoisomeric forms of the appetite suppressant, metabolic rate enhancer, or HMG-CoA reductase inhibitor of the present invention as well as mixtures thereof, including racemic mixtures, (and including those of the salts, solvates, esters and prodrugs of the compounds as well as the salts, solvates and esters of the prodrugs) form part of the present invention. In addition, the present invention embraces all geometric and positional isomers, as well as enantiomeric forms (which may exist even in the absence of asymmetric carbons), rotameric forms, atropisomers (e.g., substituted biaryls), and diastereomeric forms. For example, if the appetite suppressant, metabolic rate enhancer, or HMG-CoA reductase inhibitor of the present invention incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of a chiral HPLC column.

The present invention also embraces isotopically-labeled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively

Certain isotopically labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present invention can generally be prepared by following procedures analogous to those disclosed in the Schemes and/or in the Examples herein below, by substituting an appropriate isotopically labeled reagent for a non-isotopically labeled reagent.

Polymorphic forms of the appetite suppressant, metabolic rate enhancer, or HMG-CoA reductase inhibitor of the present invention, and of the salts, solvates, esters and prodrugs of the appetite suppressant or metabolic rate enhancer of the present invention, are intended to be included in the present invention.

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

The compounds of the present invention, or pharmaceutically acceptable salts, solvates, or esters thereof are useful in treating obesity or obesity related disorders.

The appetite suppressant, metabolic rate enhancer, or HMG-CoA reductase inhibitor of the present invention, or pharmaceutically acceptable salts, solvates, or esters thereof, can be administered in any suitable form, e.g., alone, or in combination with a pharmaceutically acceptable carrier, excipient or diluent in a pharmaceutical composition, according to standard pharmaceutical practice. The compounds of the present invention, or pharmaceutically acceptable salts, solvates, or esters thereof, can be administered orally or parenterally, including intravenous, intramuscular, interperitoneal, subcutaneous, rectal, or topical routes of administration.

Pharmaceutical compositions comprising the appetite suppressant or metabolic rate enhancer of the present invention, or a pharmaceutically acceptable salt, solvate, ester, or tautomer thereof can be in a form suitable for oral administration, e.g., as tablets, troches, capsules, lozenges, aqueous or oily suspensions, dispersible powders or granules, emulsions, syrups, or elixirs. Oral compositions may be prepared by any conventional pharmaceutical method, and may also contain sweetening agents, flavoring agents, coloring agents, and preserving agents.

The amount of the appetite suppressant or metabolic rate enhancer of the present invention, or a pharmaceutically acceptable salt, solvate, ester, or tautomer thereof, administered to a patient can be determined by a physician based on the age, weight, and response of the patient, as well as by the severity of the condition treated. For example, the amount of the appetite suppressant or metabolic rate enhancer of the present invention, or a pharmaceutically acceptable salt, solvate, ester, or tautomer thereof, administered to the patient can range from about 0.1 mg/kg body weight per day to about 60 mg/kg/d, preferably about 0.5 mg/kg/d to about 40 mg/kg/d.

Non-limiting examples of HMG CoA reductase inhibitor compounds useful in combination with the nicotinic acid receptor agonists of the present invention are lovastatin (for example MEVACOR® which is available from Merck & Co.), simvastatin (for example ZOCOR® which is available from Merck & Co.), pravastatin (for example PRAVACHOL® which is available from Bristol Meyers Squibb), atorvastatin (for example LIPITOR® which is available from Pfizer), atorvastatin, fluvastatin (for examples LESCOL® which is available from Novartis), cerivastatin, Cl-981, rivastatin (sodium 7-(4-fluorophenyl)-2,6-diisopropyl-5-methoxymethylpyridin-3-yl)-3,5-dihydroxy-6-heptanoate), rosuvastatin calcium (CRESTOR® from AstraZeneca Pharmaceuticals), pitavastatin (such as NK-104 of Negma Kowa of Japan).

H₃ receptors have been implicated in thermogenesis regulation in rodents and in feeding behavior in humans. Various H₃ receptor antagonists/inverse agonists have been disclosed as useful for modulating histaminergic function, and thereby can be useful in treating obesity and obesity-related conditions. H₃ receptor antagonists/inverse agonists have been disclosed in U.S. 2002/183309, 2002/177589, 2002/111340, 2004/0122033, 2003/0186963, 2003/0130253, 2004/0248938, 2002/0058659, 2003/0135056, 2003/134835, 2003/153548, 2004/0019099, 2004/0097483, 2004/0048843, 2004/087573, 2004/092521, 2004/214856, 2004/248899, 2004/224953, 2004/224952, 2005/222151, 2005/222129, 2005/182045, 2005/171181, U.S. Pat. Nos. 6,620,839, 6,515,013, 6,559,140, 6,316,475, 6,166,060, 6,448,282, 6,008,240, 5,652,258, 6,417,218, 6,673,829, 6,756,384, 6,437,147, 6,720,328, 5,869,479, 6,849,621, 6,908,929, 6,908,926, 6,906,060, 6,884,809, 6,884,803, 6,878,736, 6,638,967, 6,610,721, 6,528,522, 6,518,287, 6,506,756, 6,489,337, 6,436,939, 6,448,282, 6,407,132, 6,355,665, 6,248,765, 6,133,291, 6,103,735, 6,080,871, 5,932,596, 5,929,089, 5,837,718, 5,821,259, 5,807,872, 5,639,775, 5,708,171, 5,578,616, 5,990,147, 6,906,081, WO 95/14007, WO 99/24405 (each of which is herein incorporated by reference).

In one embodiment, the present invention is directed to compositions comprising one or more metabolic rate enhancer which is an H₃ receptor antagonist/inverse agonist described generically (i.e., a compound according to Formula (I)-(VIII) as described herein) or specifically exemplified in U.S. Pat. Nos. 6,720,328, 6,849,621, 2004/0019099, 2004/0097483, 2004/0048843, or 2005/0113383 (each of which is herein incorporated by reference); and one or more appetite suppressant selected from the group consisting of a CB₁ antagonist (e.g., rimonabant), phentermine, sibutramine, and topiramate.

In another embodiment, the present invention is directed to compositions comprising one or more H₃ receptor antagonist/inverse agonist; one or more appetite suppressant selected from the group consisting of a CB₁ antagonist (e.g., rimonabant), phentermine, sibutramine, and topiramate; and one or more HMG-CoA reductase inhibitor.

In another embodiment, the present invention is directed to compositions comprising one or more H₃ receptor antagonist/inverse agonists and one or more anti-diabetic agents. The compositions are useful for treating or preventing diabetes.

There are two major forms of diabetes: Type I diabetes (also referred to as insulin-dependent diabetes or IDDM) and Type II diabetes (also referred to as noninsulin dependent diabetes or NIDDM). In one embodiment, the compositions are useful for treating Type I diabetes. In another embodiment, the compositions are useful for treating Type II diabetes.

Examples of anti-diabetic agents useful in the present methods for treating diabetes include sulfonylureas, insulin sensitizers (such as PPAR agonists, DPPIV inhibitors, PTP-1B inhibitors and glucokinase activators), α-glucosidase inhibitors, insulin secretagogues, hepatic glucose output lowering compounds, anti-obesity agents, antihypertensive agents, meglitinides, insulin and insulin-containing compositions.

In one embodiment, the anti-diabetic agent is an insulin sensitizer or a sulfonylurea.

Non-limiting examples of sulfonylureas include glipizide, tolbutamide, glyburide, glimepiride, chlorpropamide, acetohexamide, gliamilide, gliclazide, glibenclamide and tolazamide. Insulin sensitizers include PPAR-γ agonists described in detail above, preferably troglitazone, rosiglitazone, pioglitazone and englitazone; biguanidines such as mefformin and phenformin; DPPIV inhibitors such as sitagliptin, saxagliptin, denagliptin and vildagliptin; PTP-1B inhibitors; and glucokinase activators. α-Glucosidase inhibitors that can be useful in treating type II diabetes include miglitol, acarbose, and voglibose. Hepatic glucose output lowering drugs include Glucophage and Glucophage XR. Insulin secretagogues include sulfonylurea and non-sulfonylurea drugs such as GLP-1, exendin, GIP, secretin, glipizide, chlorpropamide, nateglinide, meglitinide, glibenclamide, repaglinide and glimepiride. Insulin includes all formualtions of insulin, including long acting and short acting forms of insulin.

Non-limiting examples of anti-obesity agents useful in the present methods for treating diabetes include CB1 antagonists or inverse agonists such as rimonabant, neuropeptide Y antagonists, MCR4 agonists, MCH receptor antagonists, histamine H3 receptor antagonists or inverse agonists, leptin, appetite suppressants such as sibutramine, and lipase inhibitors such as xenical.

Non-limiting examples of antihypertensive agents useful in the present methods for treating diabetes include β-blockers and calcium channel blockers (for example diltiazem, verapamil, nifedipine, amlopidine, and mybefradil), ACE inhibitors (for example captopril, lisinopril, enalapril, spirapril, ceranopril, zefenopril, fosinopril, cilazopril, and quinapril), AT-1 receptor antagonists (for example losartan, irbesartan, and valsartan), renin inhibitors and endothelin receptor antagonists (for example sitaxsentan).

Non-limiting examples of meglitinides useful in the present methods for treating diabetes include repaglinide and nateglinide.

Non-limiting examples of insulin sensitizers include biguanides, such as mefformin and thiazolidinediones.

In one embodiment, the insulin sensitizer is a thiazolidinedione.

Non-limiting examples of antidiabetic agents that slow or block the breakdown of starches and certain sugars and are suitable for use in the compositions and methods of the present invention include alpha-glucosidase inhibitors and certain peptides for increasing insulin production. Alpha-glucosidase inhibitors help the body to lower blood sugar by delaying the digestion of ingested carbohydrates, thereby resulting in a smaller rise in blood glucose concentration following meals. Non-limiting examples of suitable alpha-glucosidase inhibitors include acarbose; miglitol; camiglibose; certain polyamines as disclosed in WO 01/47528 (incorporated herein by reference); voglibose. Non-limiting examples of suitable peptides for increasing insulin production including amlintide (CAS Reg. No. 122384-88-7 from Amylin; pramlintide, exendin, certain compounds having Glucagon-like peptide-1 (GLP-1) agonistic activity as disclosed in WO 00/07617 (incorporated herein by reference).

Non-limiting examples of orally administrable insulin and insulin containing compositions include AL-401 from Autoimmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

In another embodiment, the compositions comprising one or more H₃ receptor antagonist/inverse agonists and one or more anti-diabetic agents are useful for treating or preventing obesity or an obesity-related disorder.

Anti-diabetic agents useful in the present methods for treating obesity or an obesity-related disorder include, but are not limited to the anti-diabetic agents listed above herein.

In the combination therapies of the present invention, the one or more H₃ receptor antagonist/inverse agonists and the one or more additional therapeutic agents can be administered simultaneously (at the same time, in a single dosage form or in separate dosage forms) or sequentially (first one and then another, etc. . . . over a period of time) in any order.

In one embodiment, the H₃ antagonists/inverse agonists of the present invention can have a structure according to Formula (I): 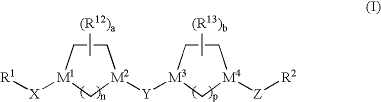
as described in U.S. Pat. No. 6,720,328, which is herein incorporated by reference in its entirety. Non-limiting examples of compounds of Formula (I) include:

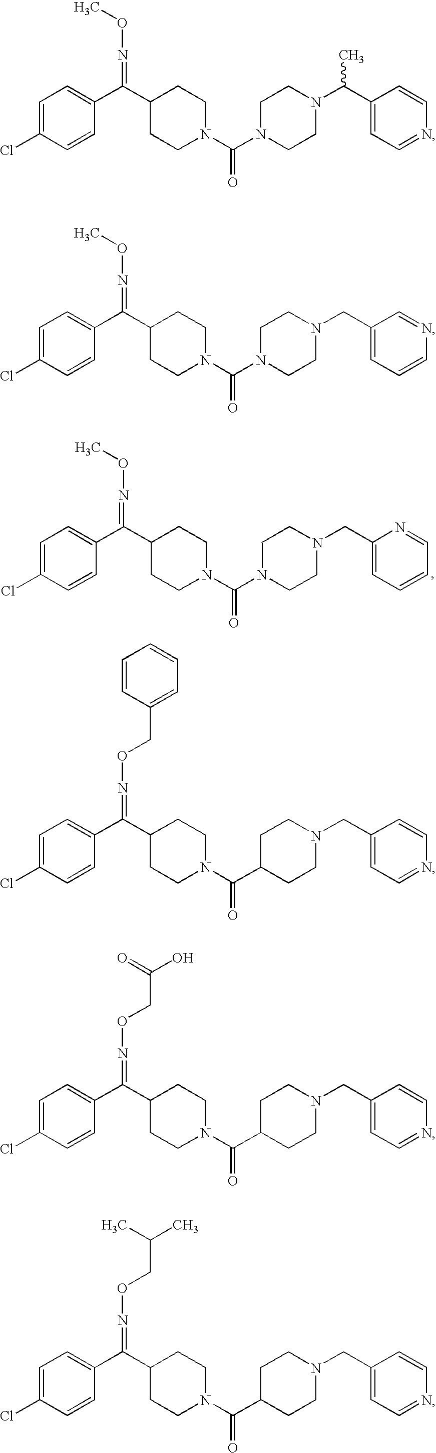

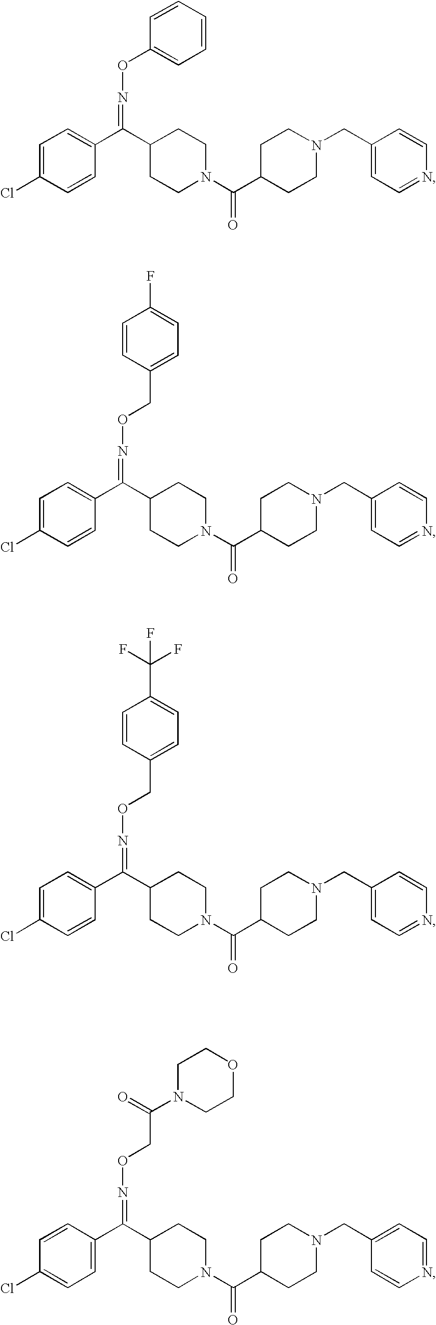

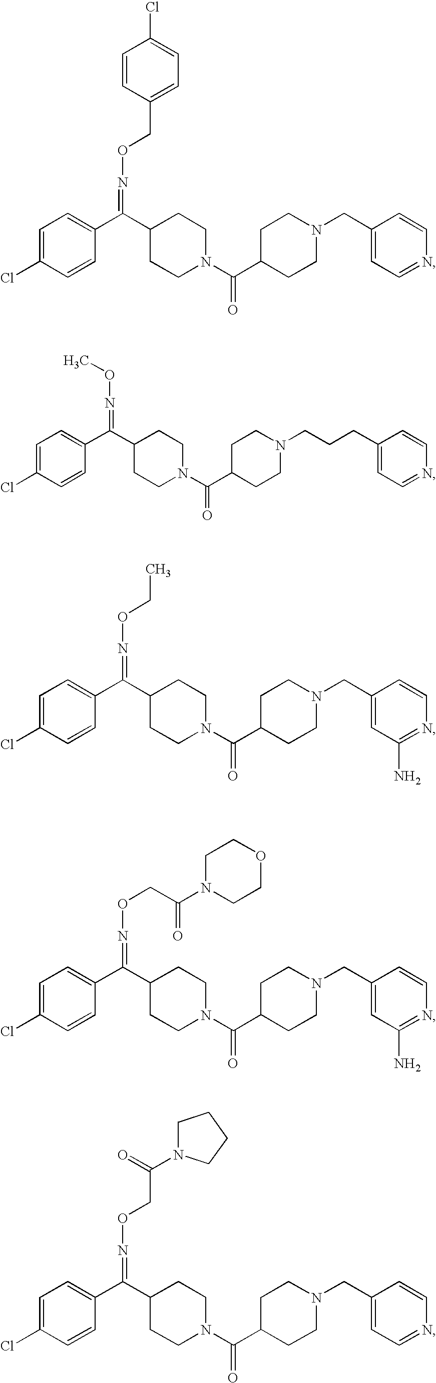

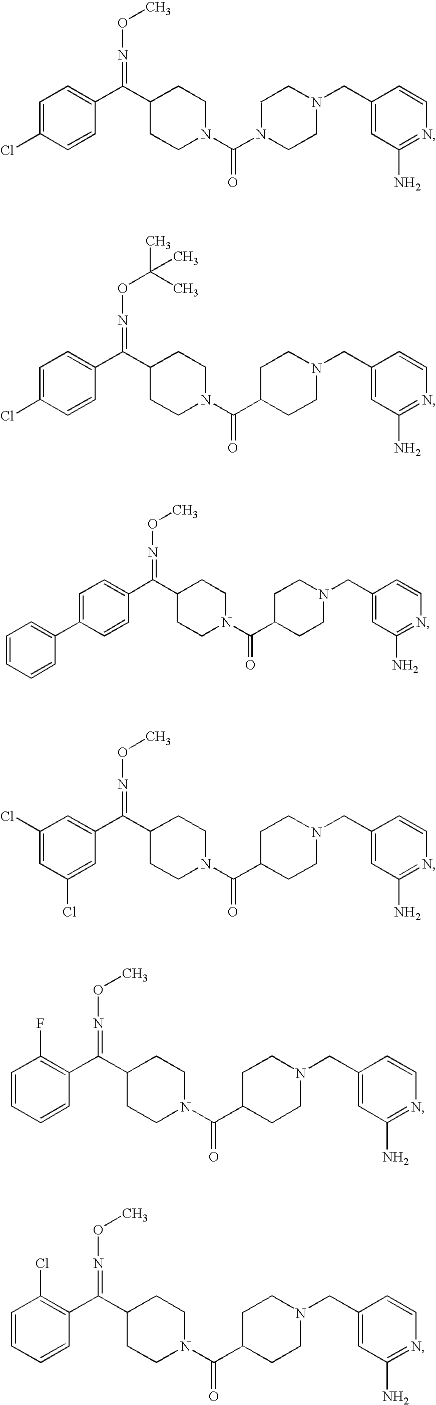

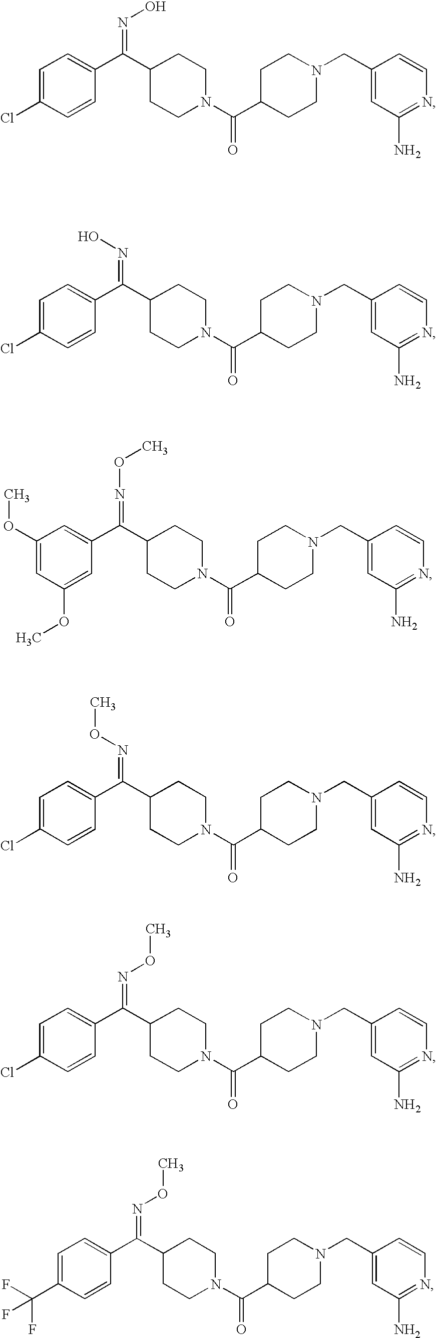

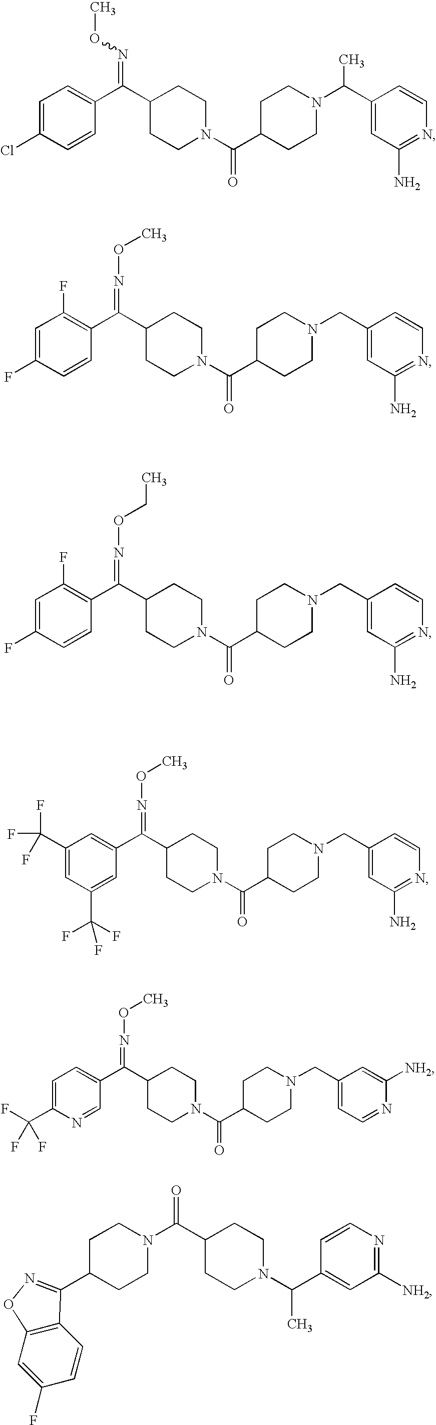

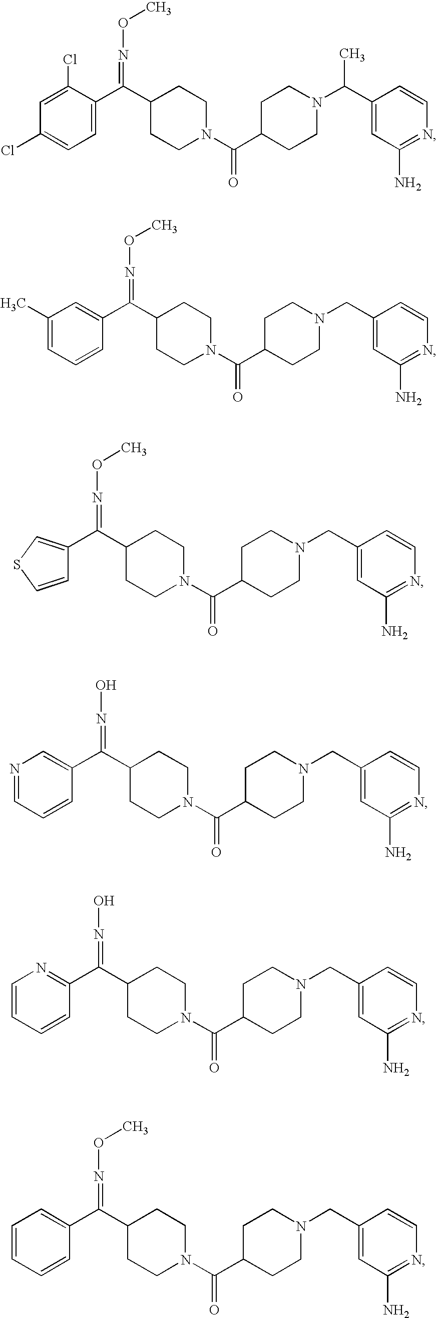

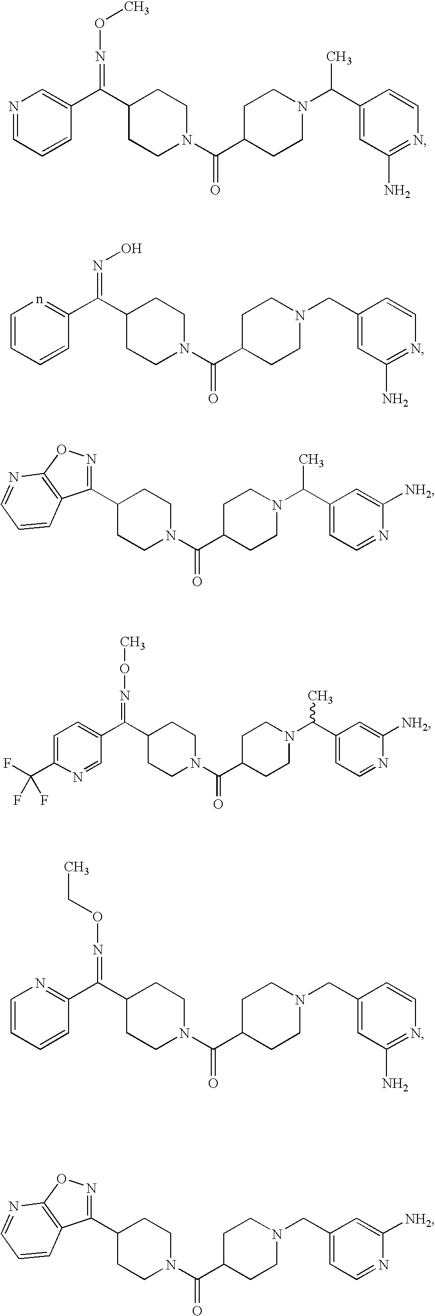

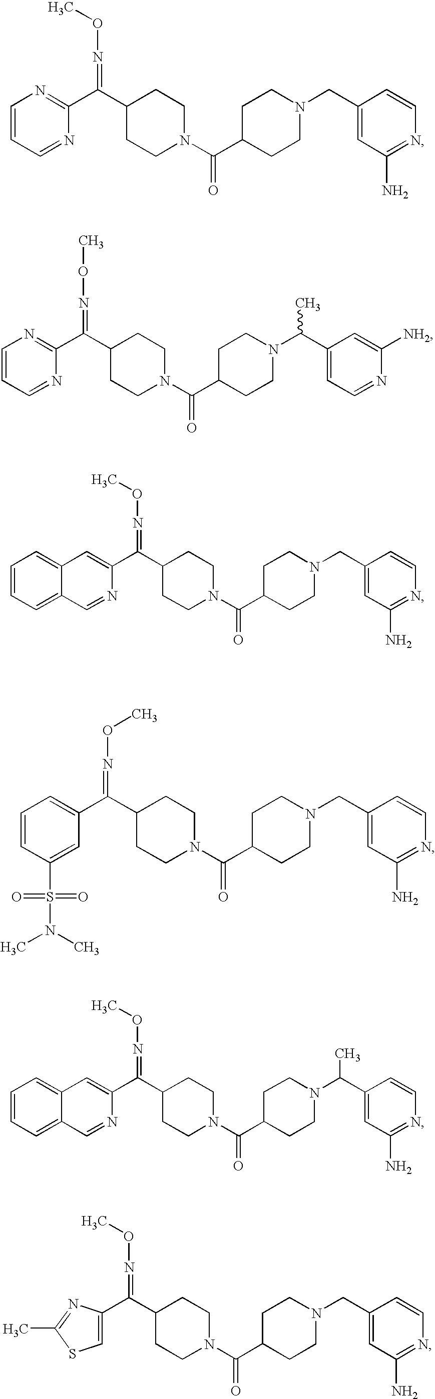

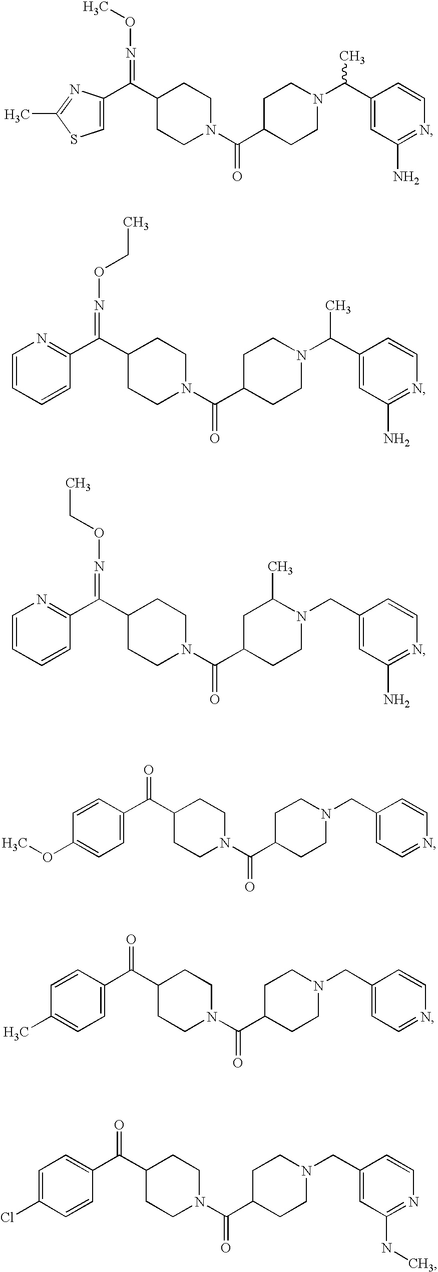

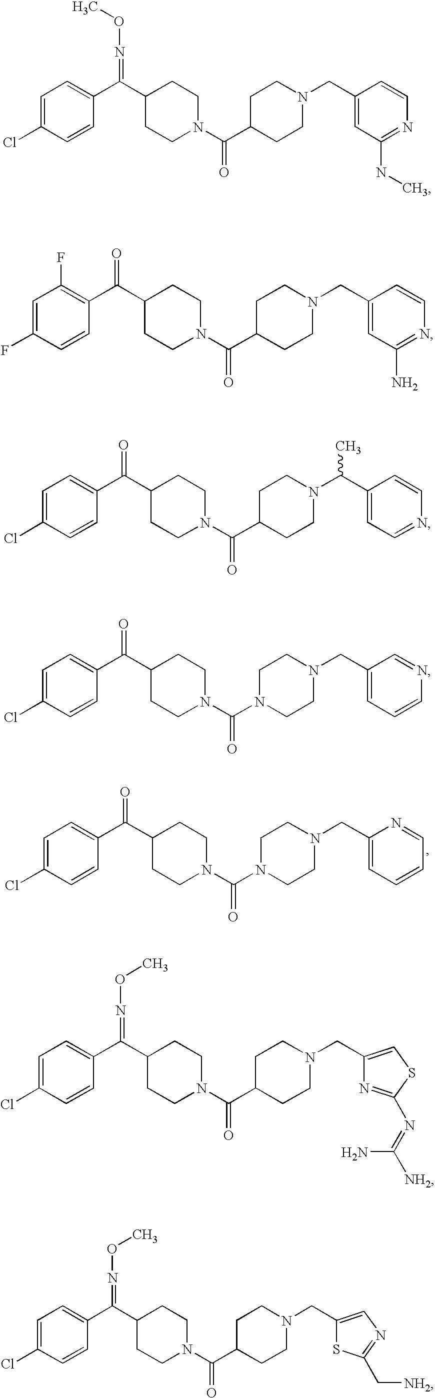

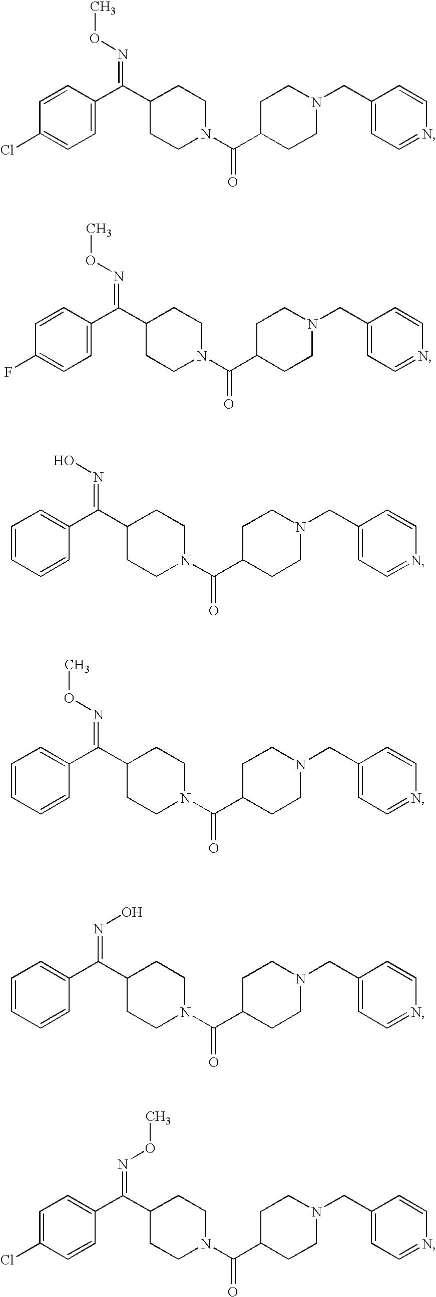

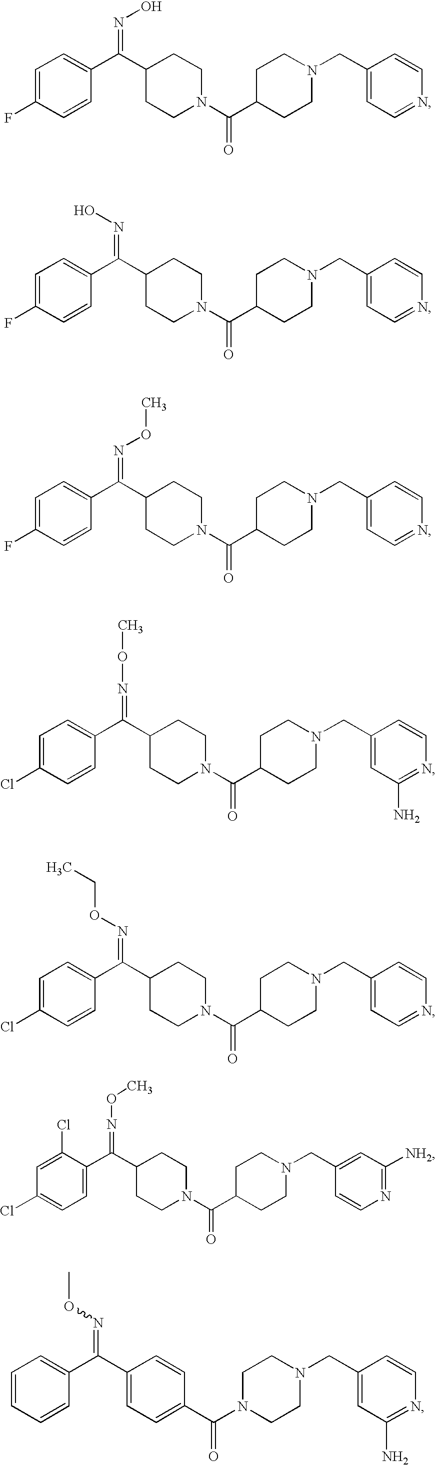

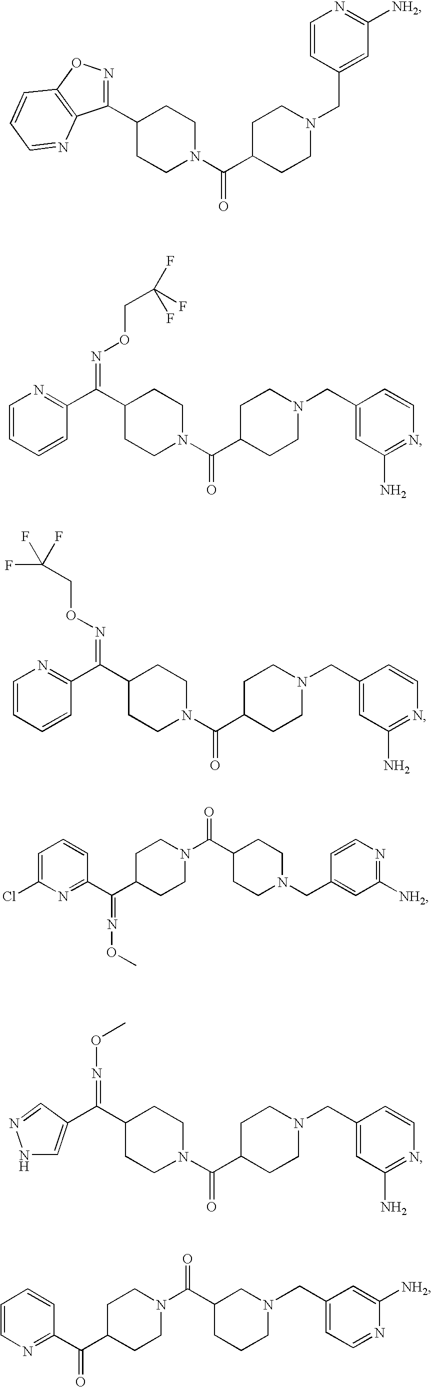

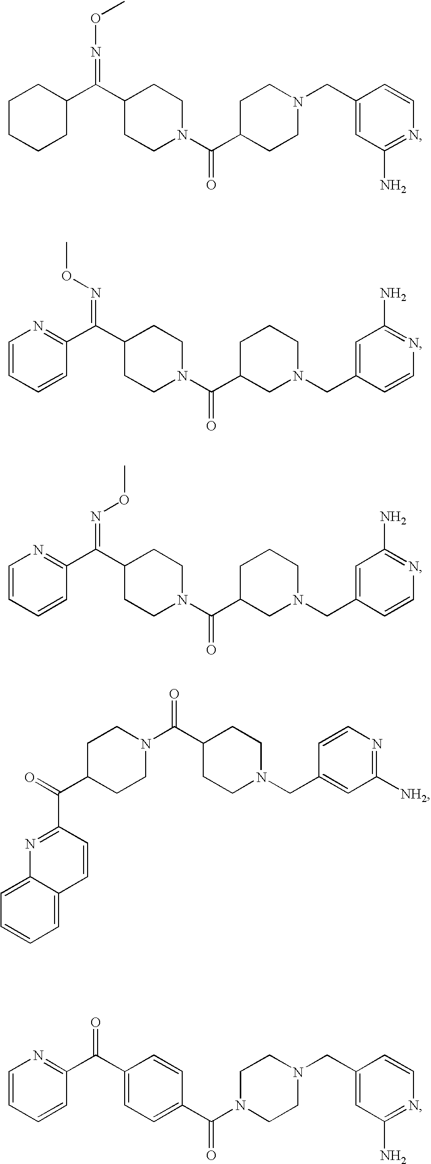

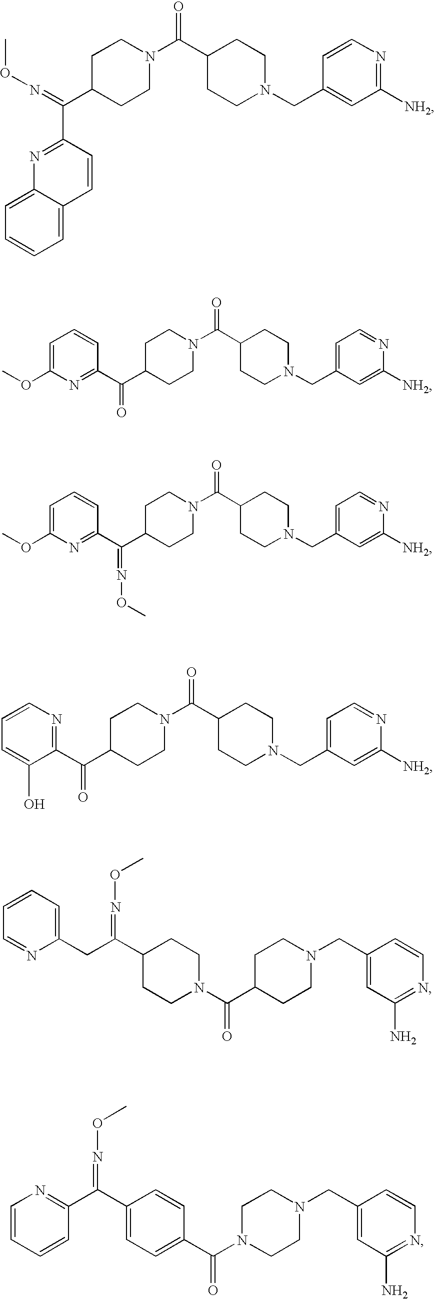

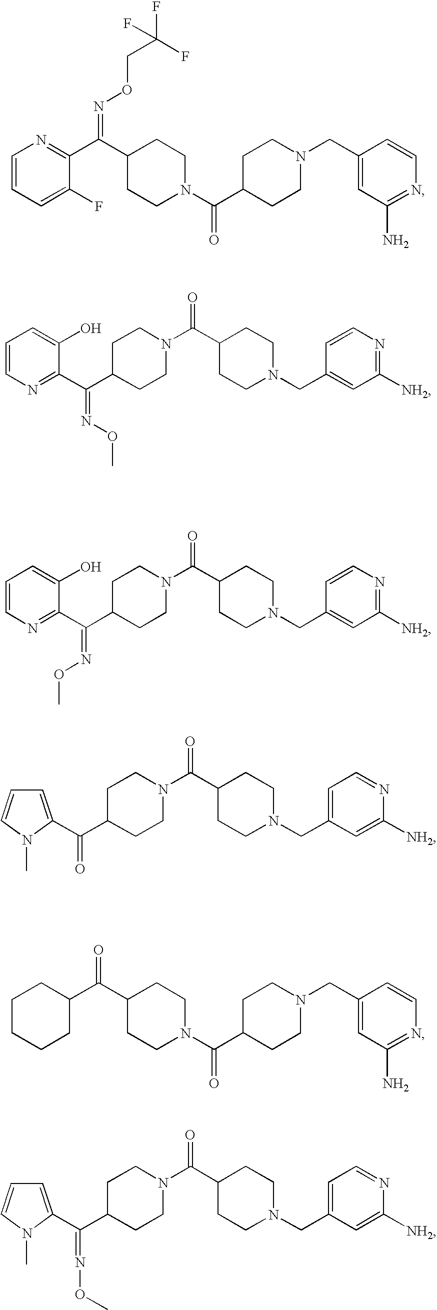

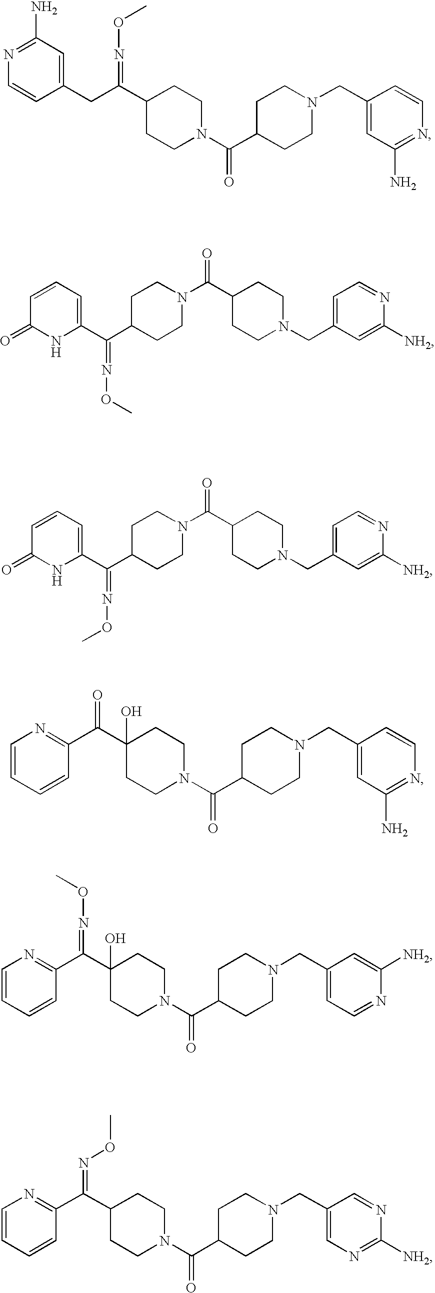

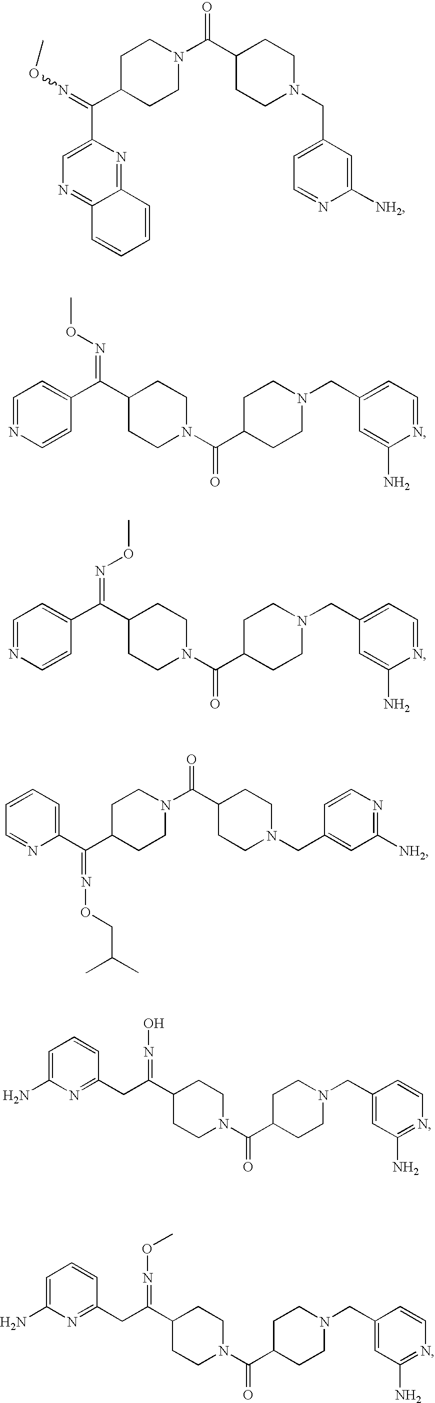

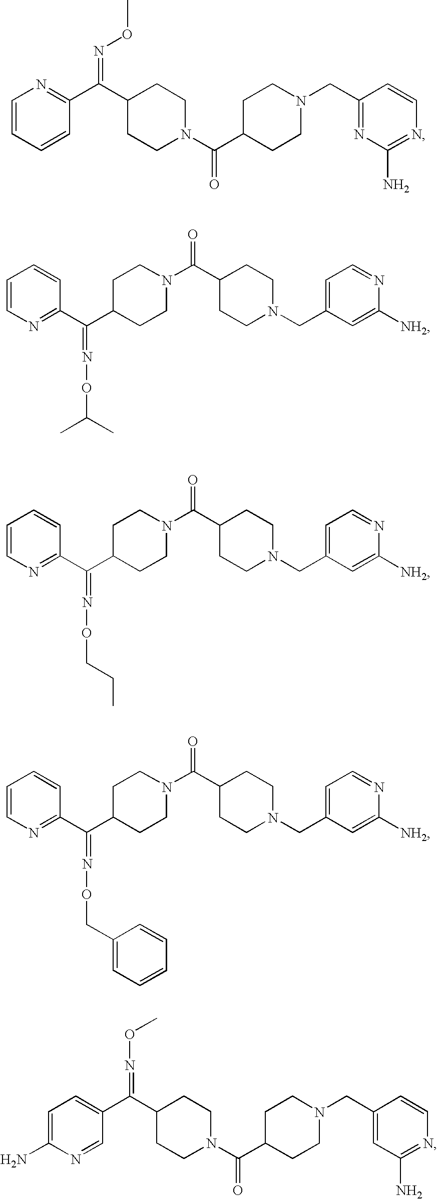

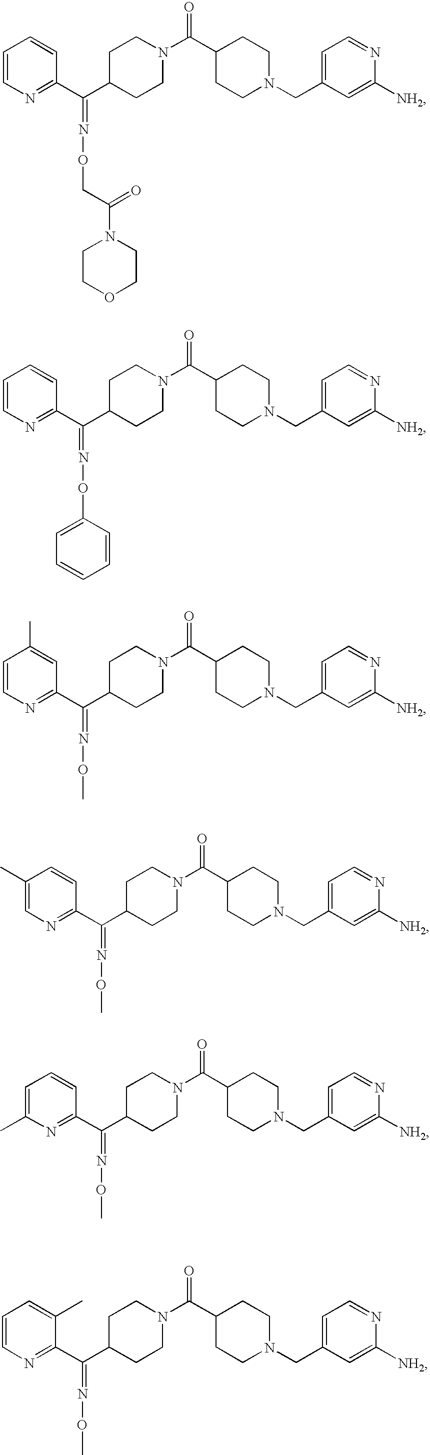

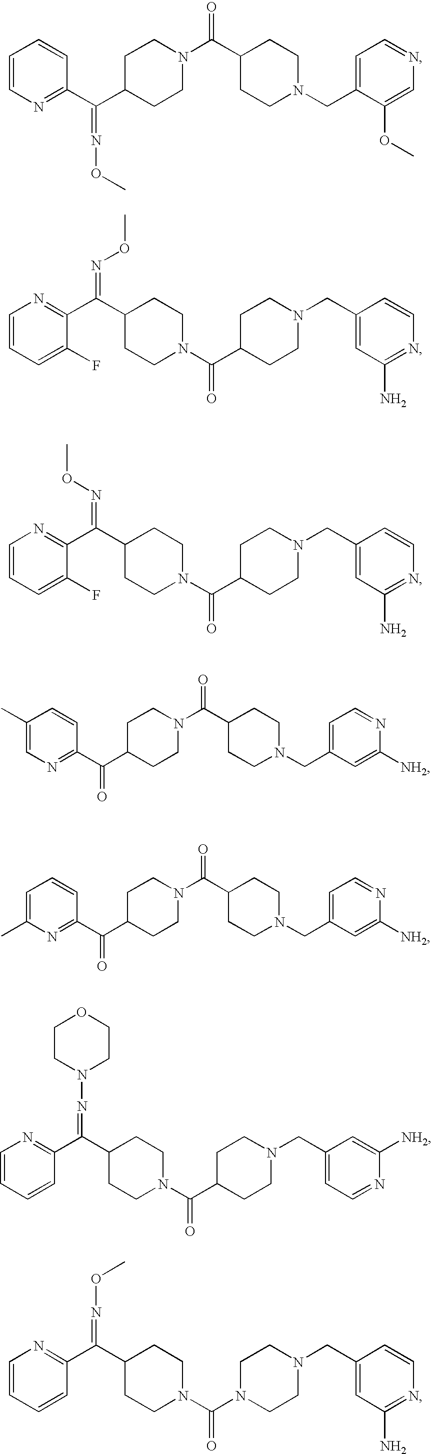

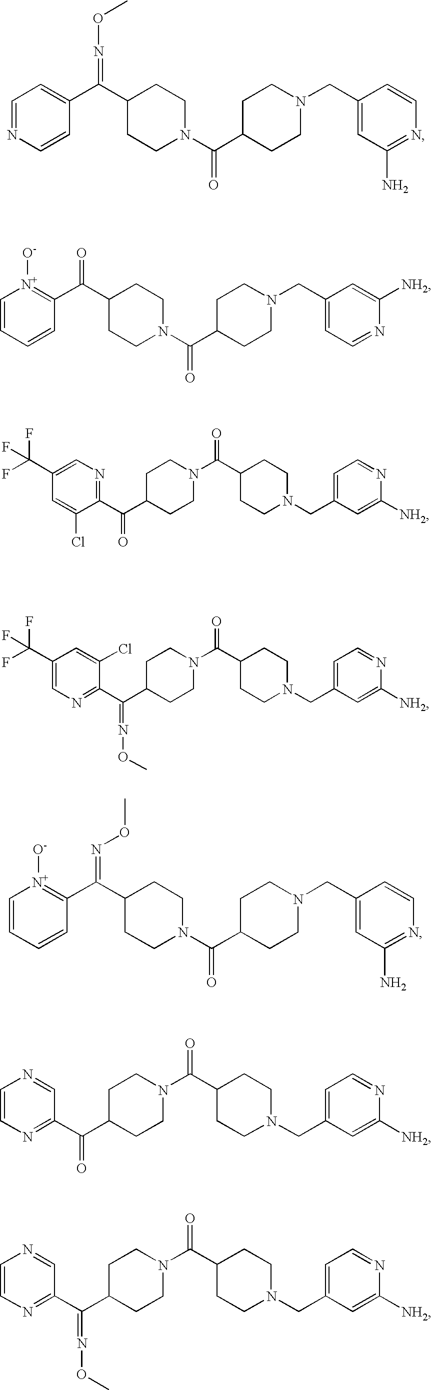

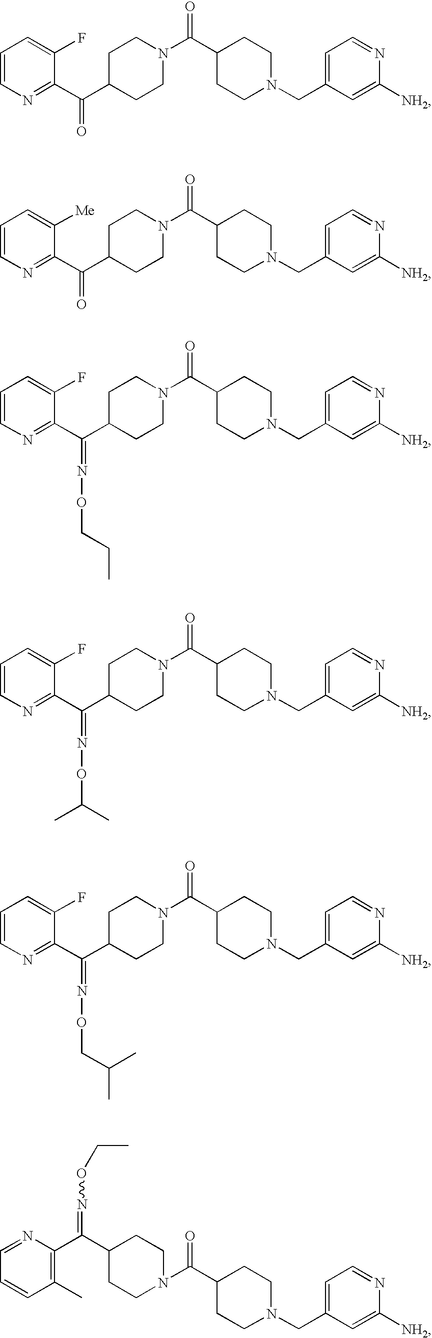

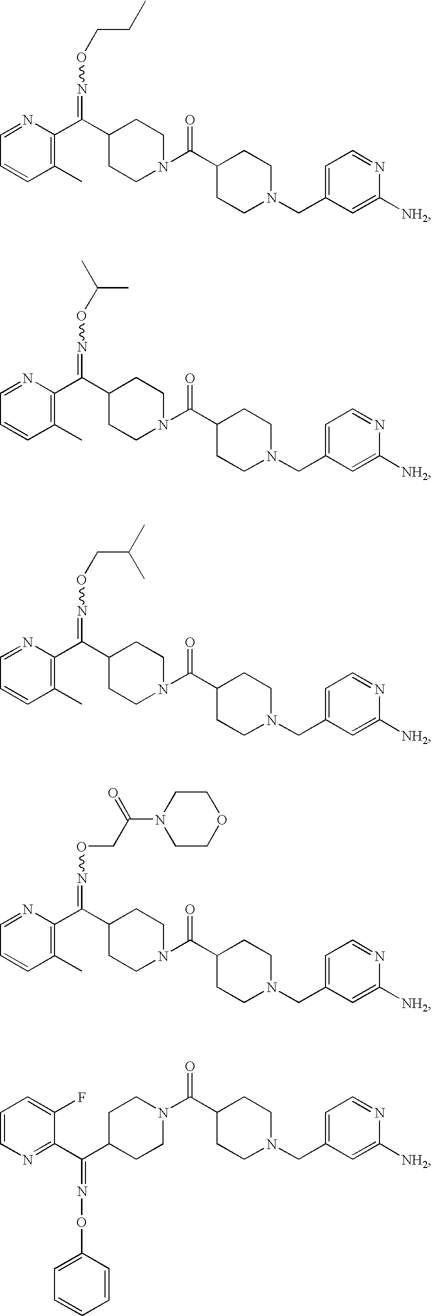

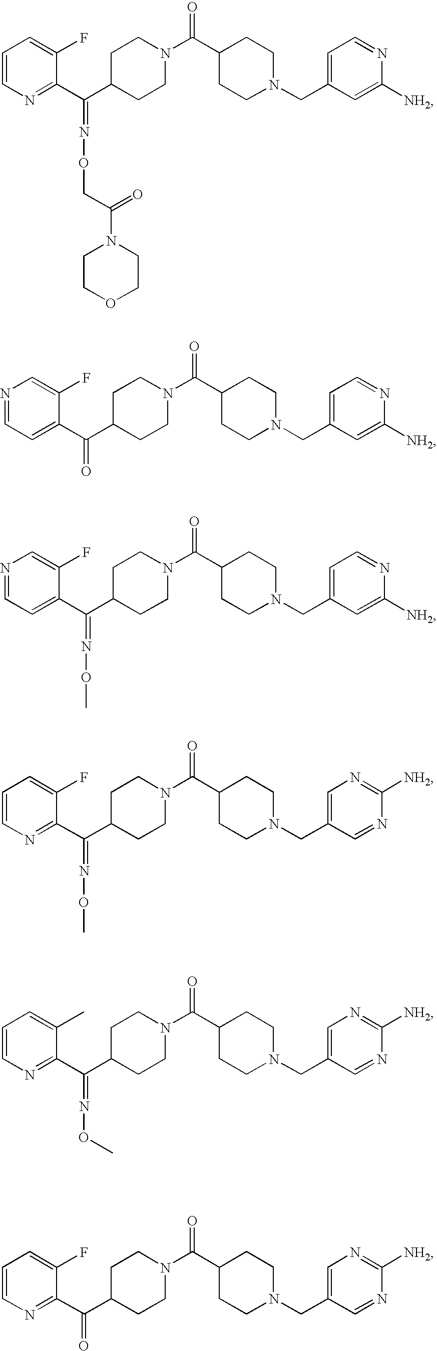

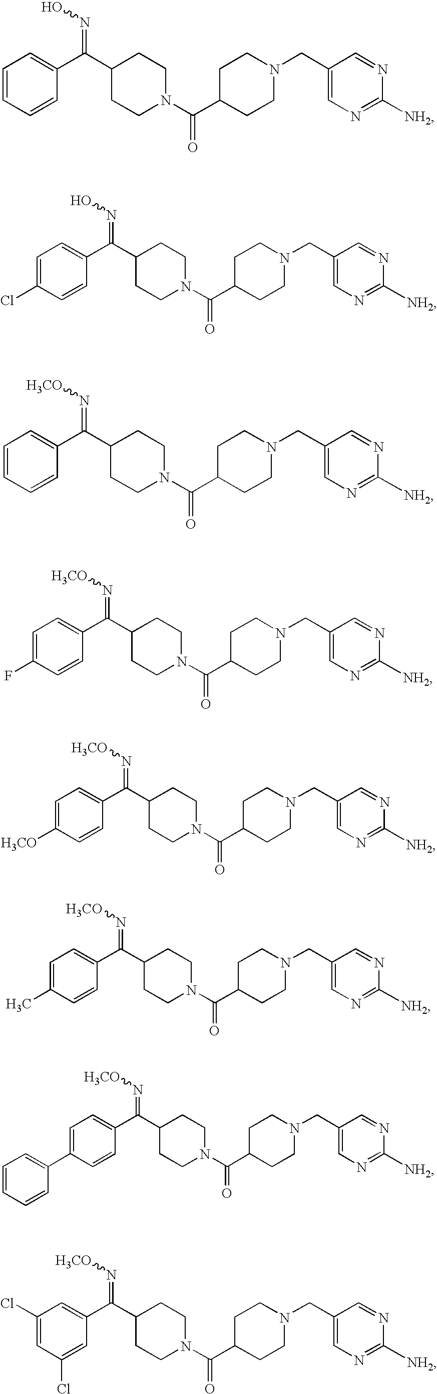

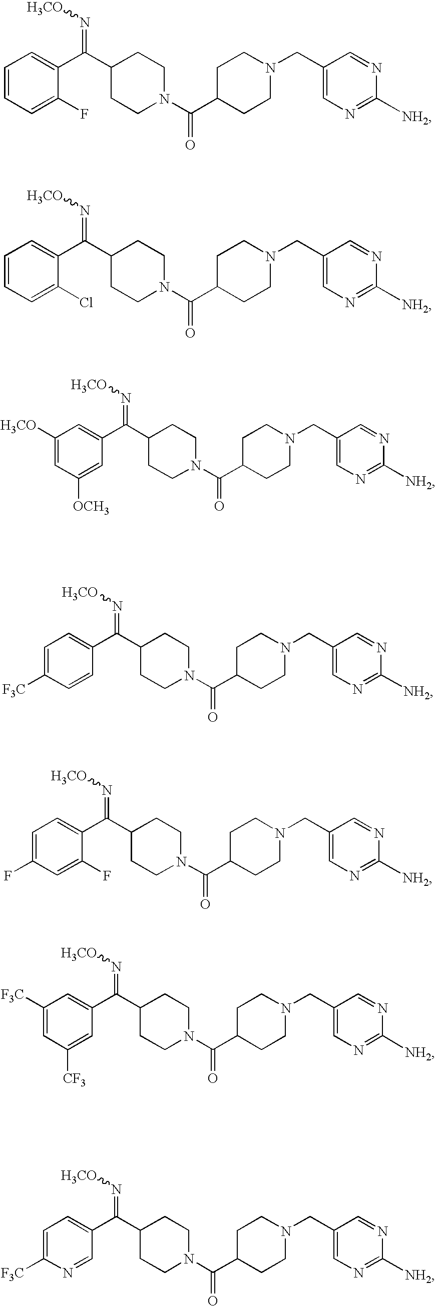

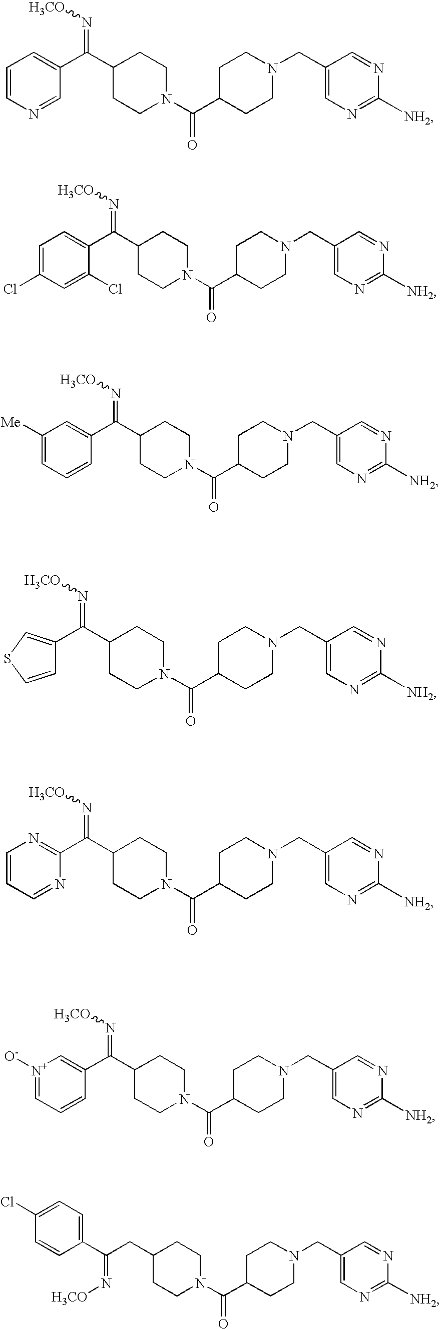

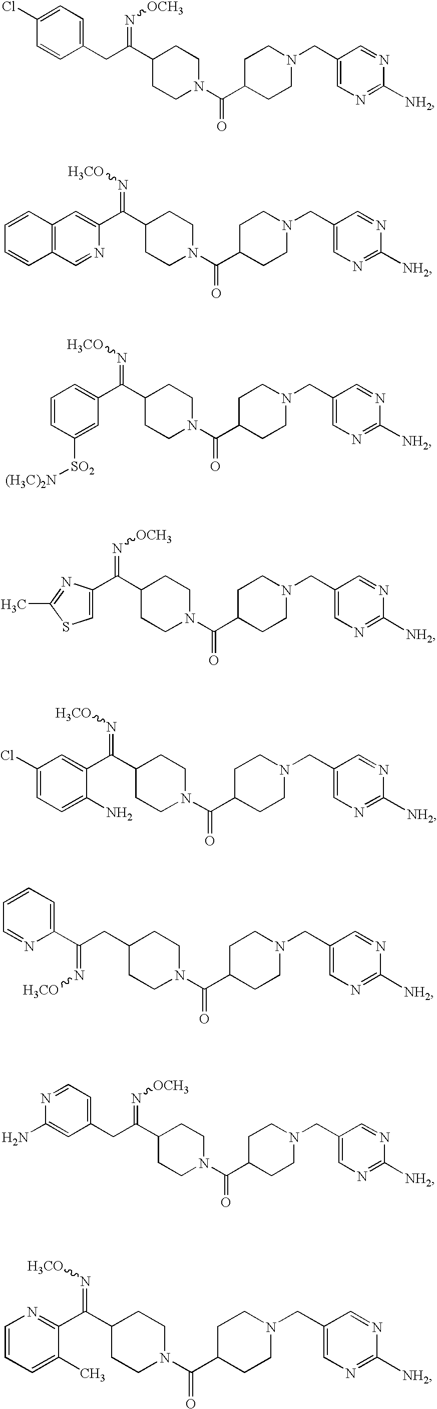

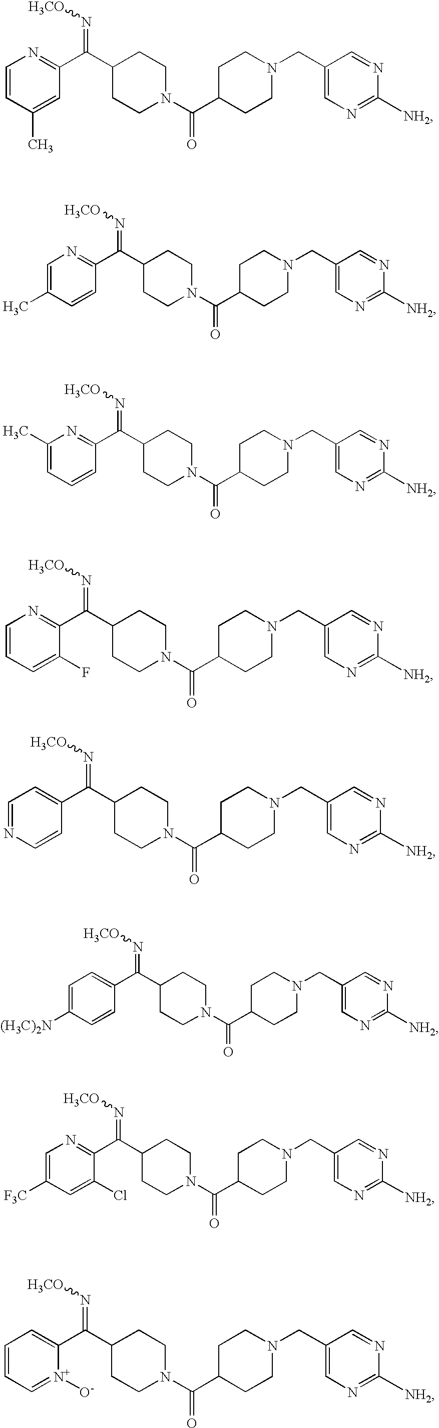

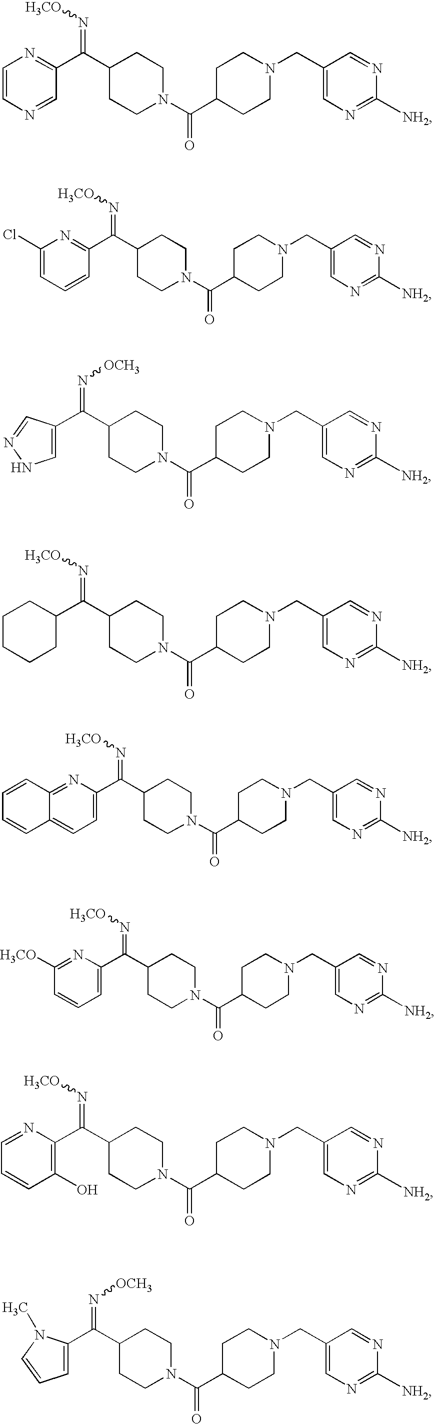

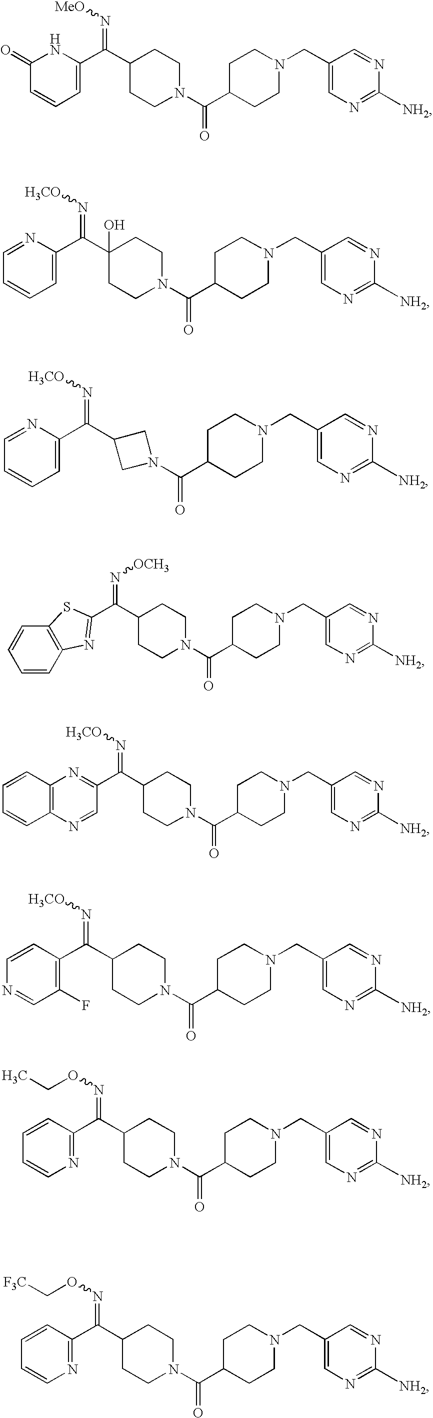

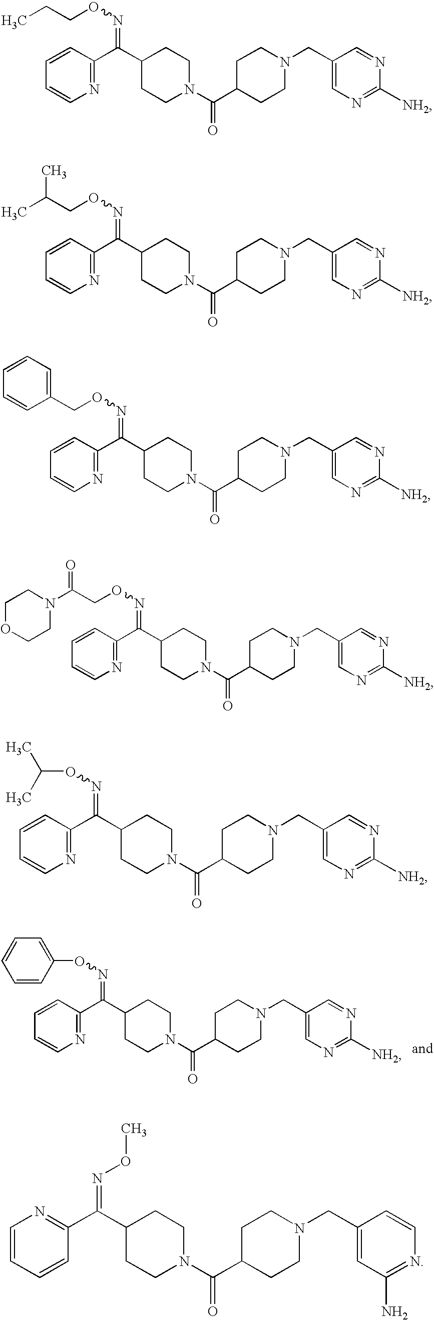

In one embodiment, the H₃ antagonists/inverse agonists of the present invention can have a structure according to Formula (II): 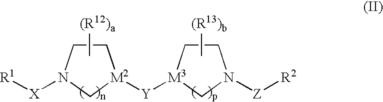
as described in U.S. Pat. No. 6,849,621 and U.S. 2005/0113383, both of which are herein incorporated by reference in their entirety. Non-limiting examples of compounds of Formula (II) include:

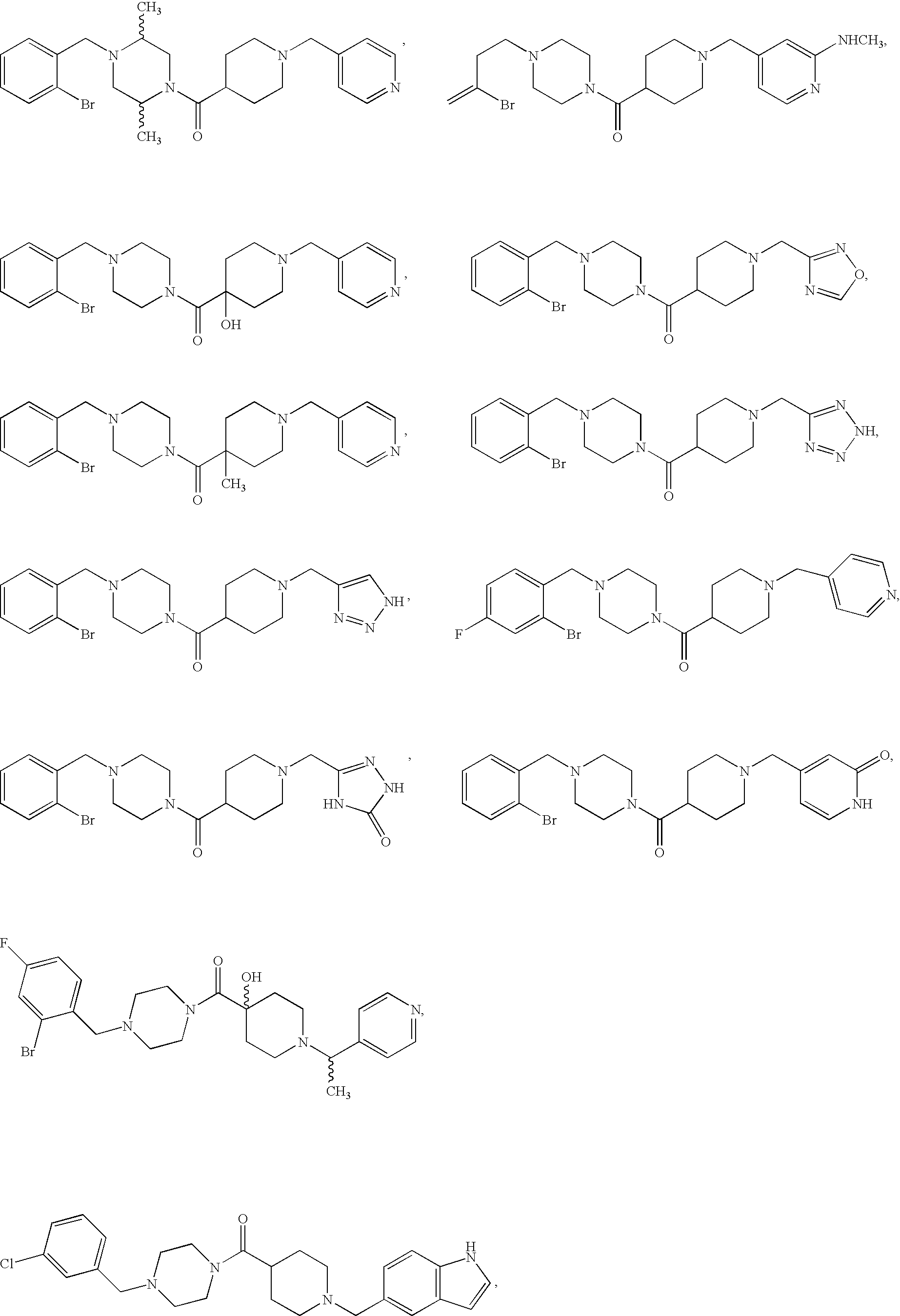

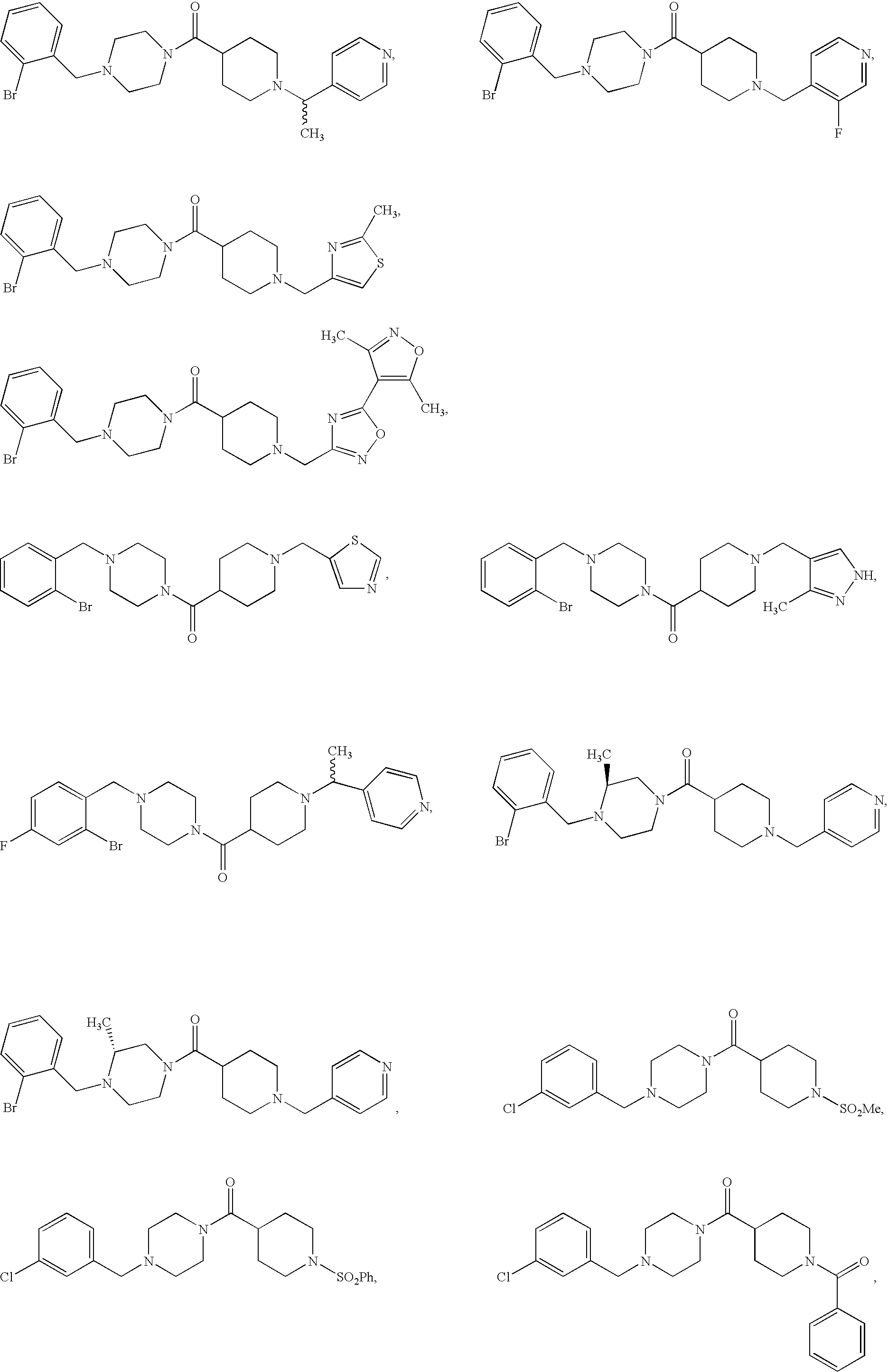

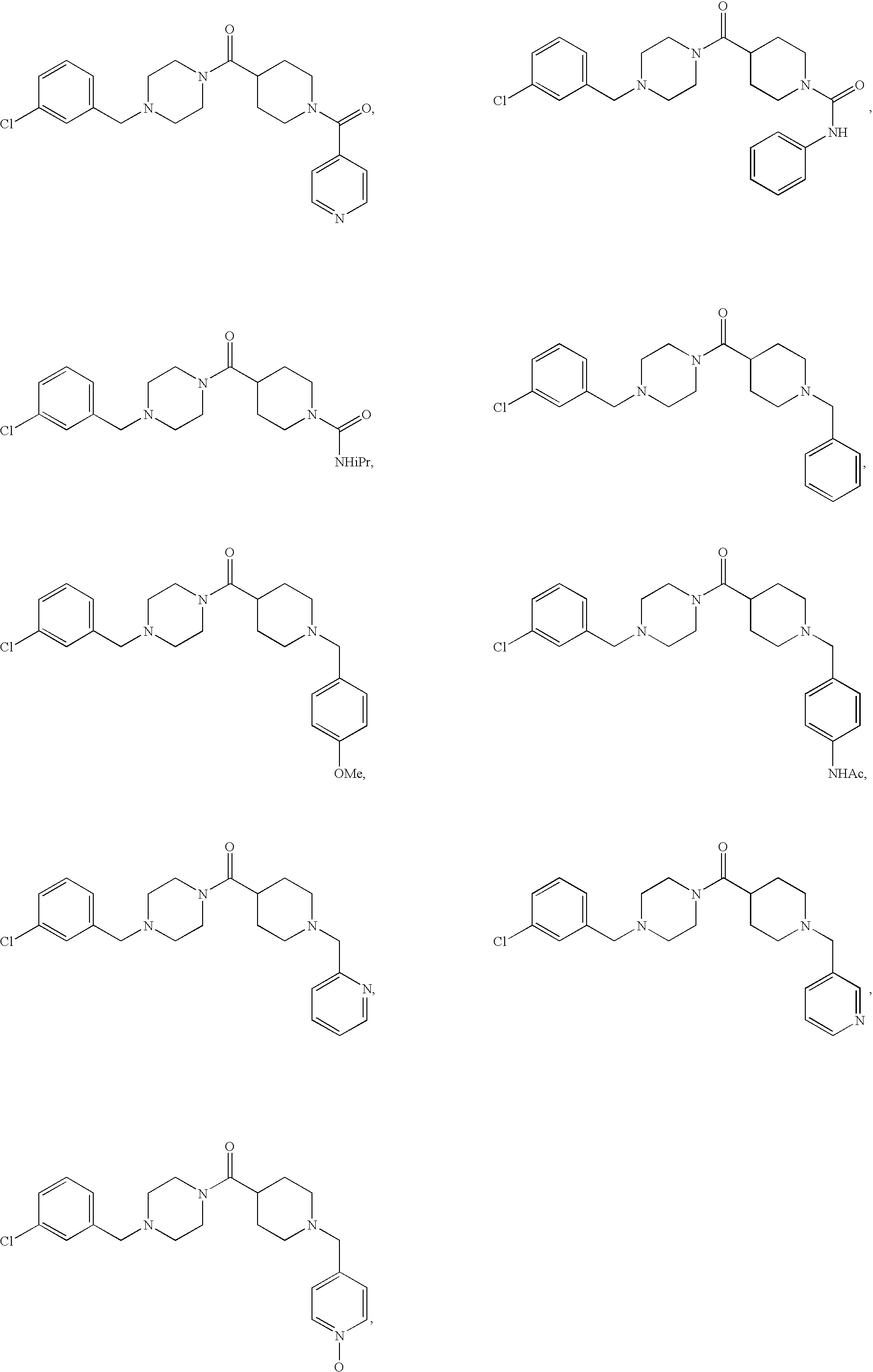

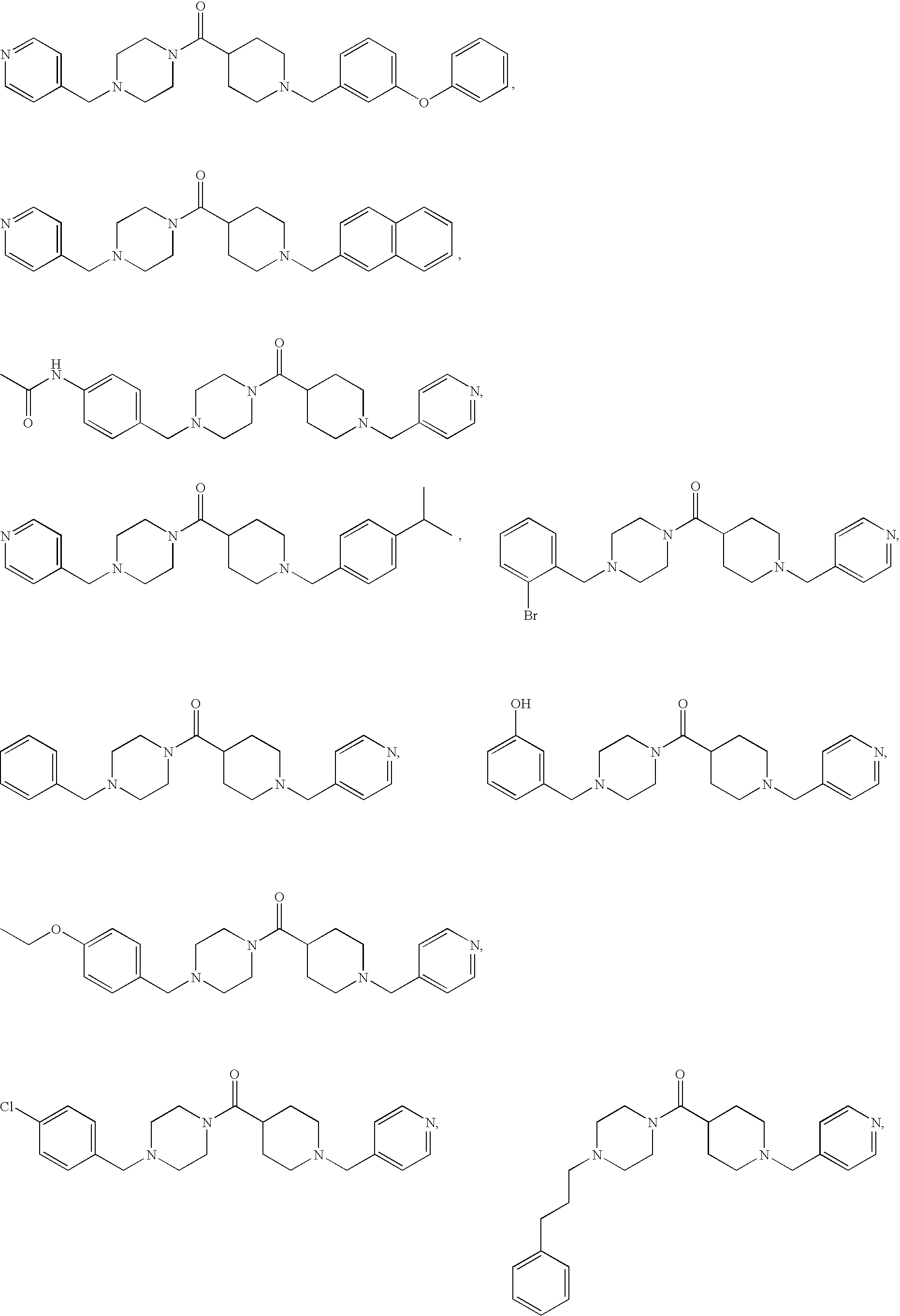

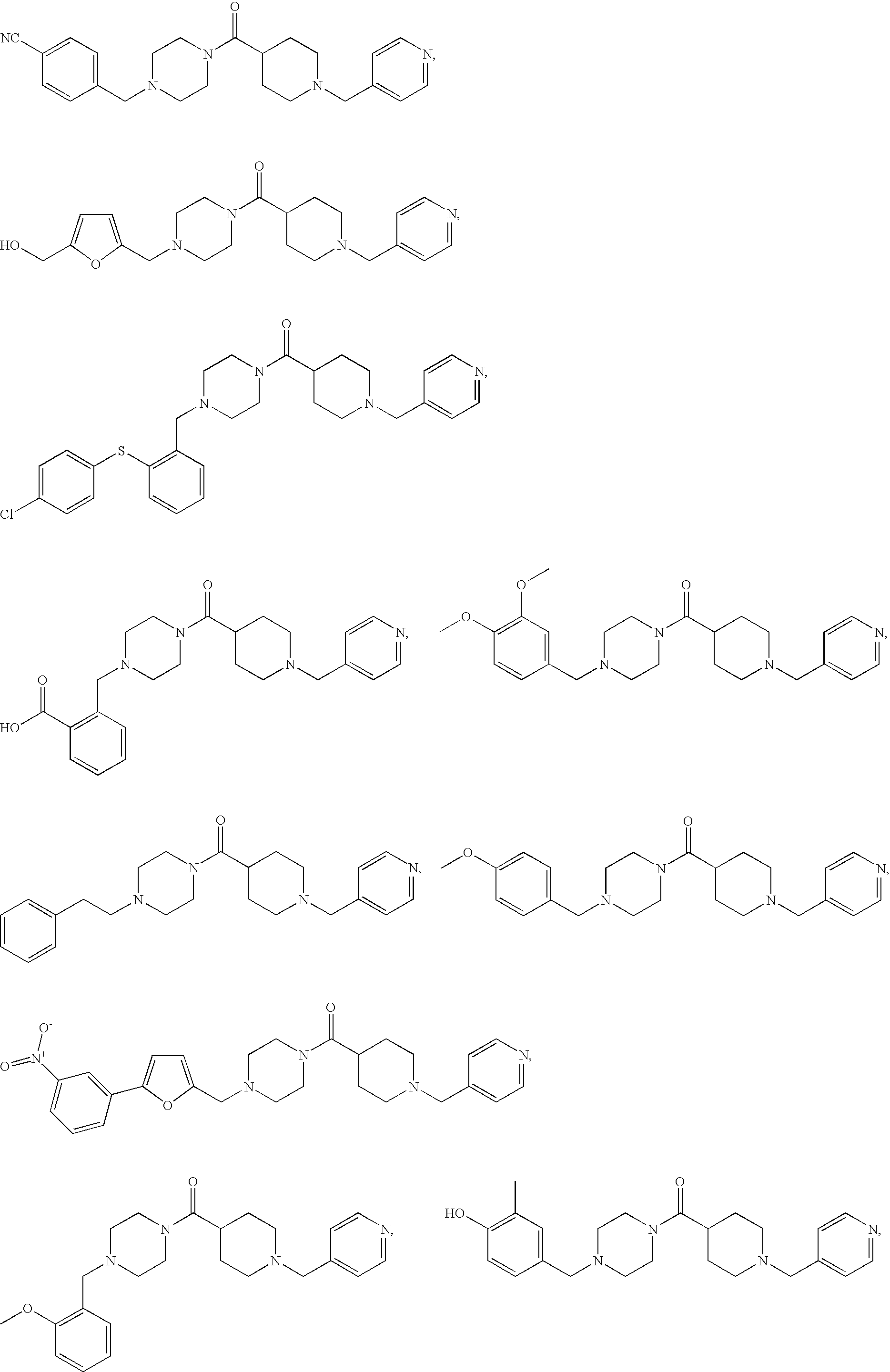

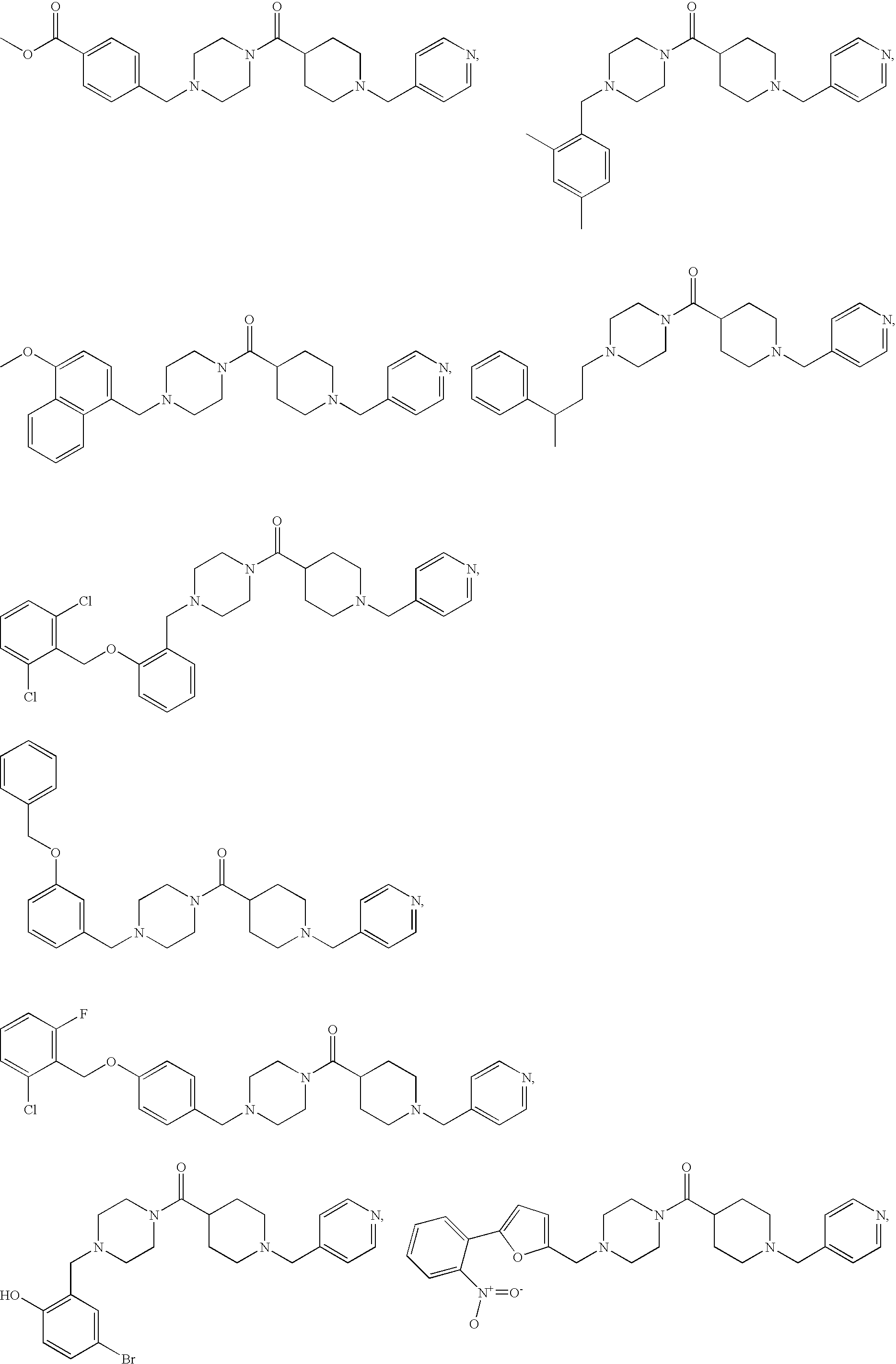

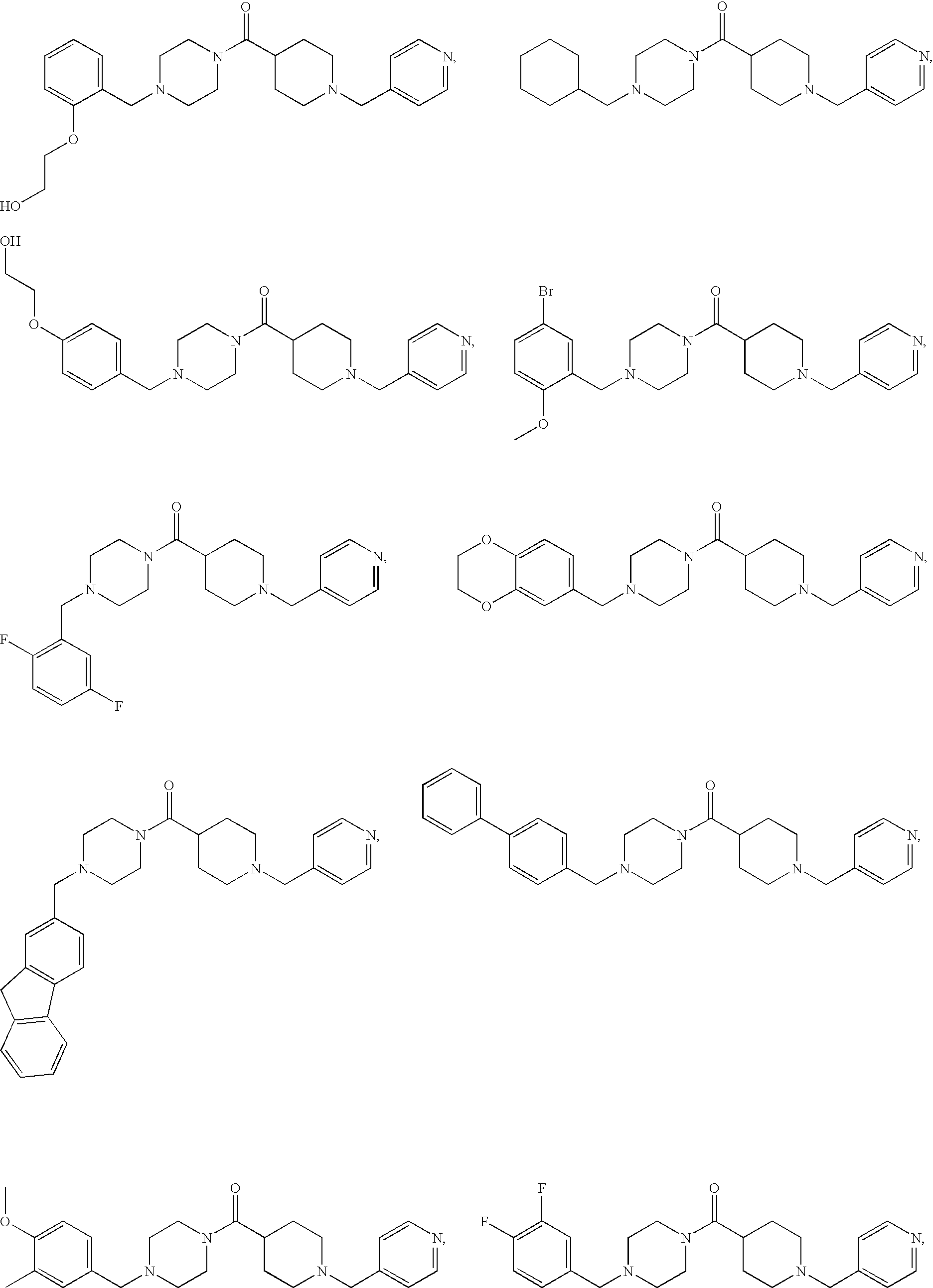

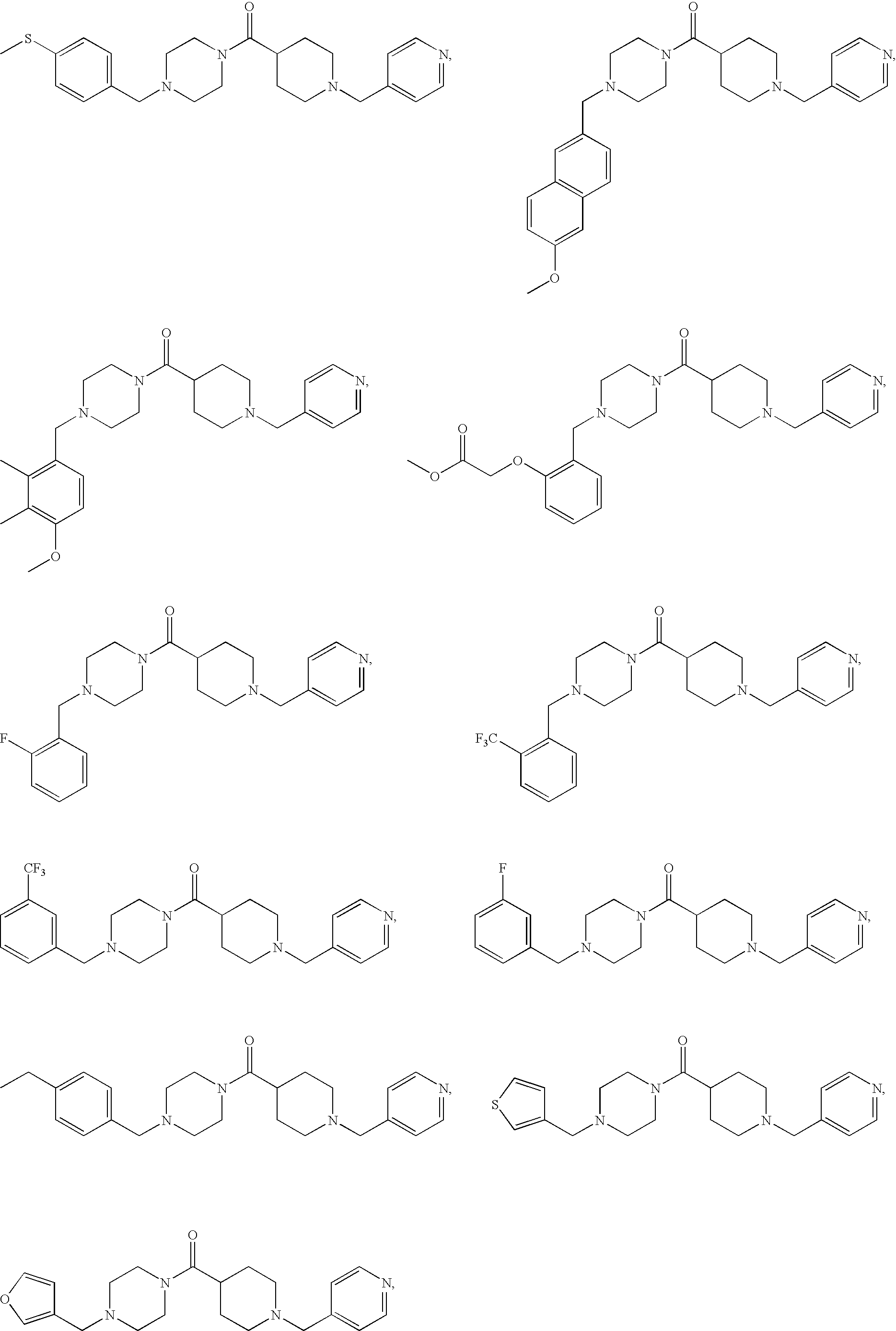

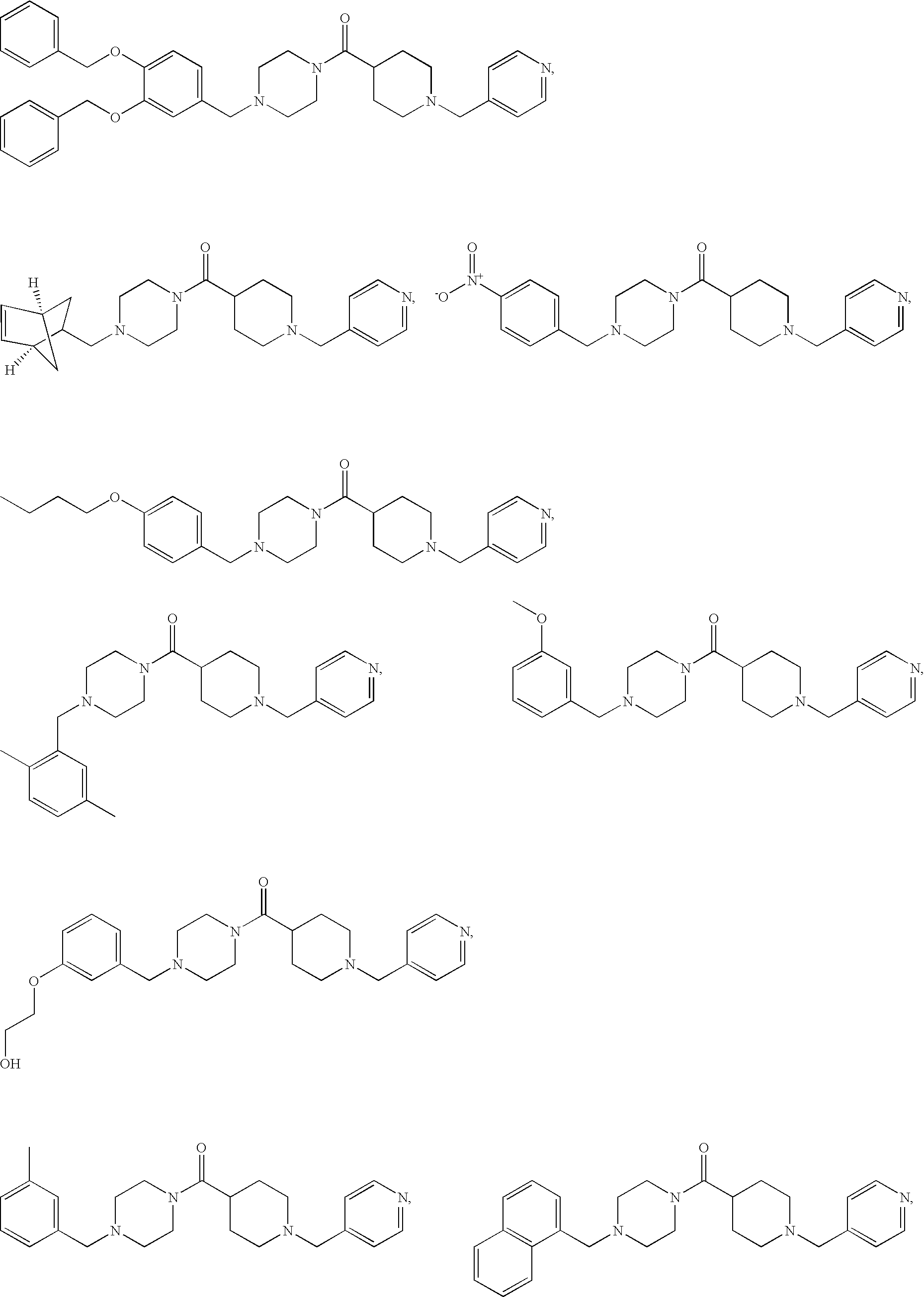

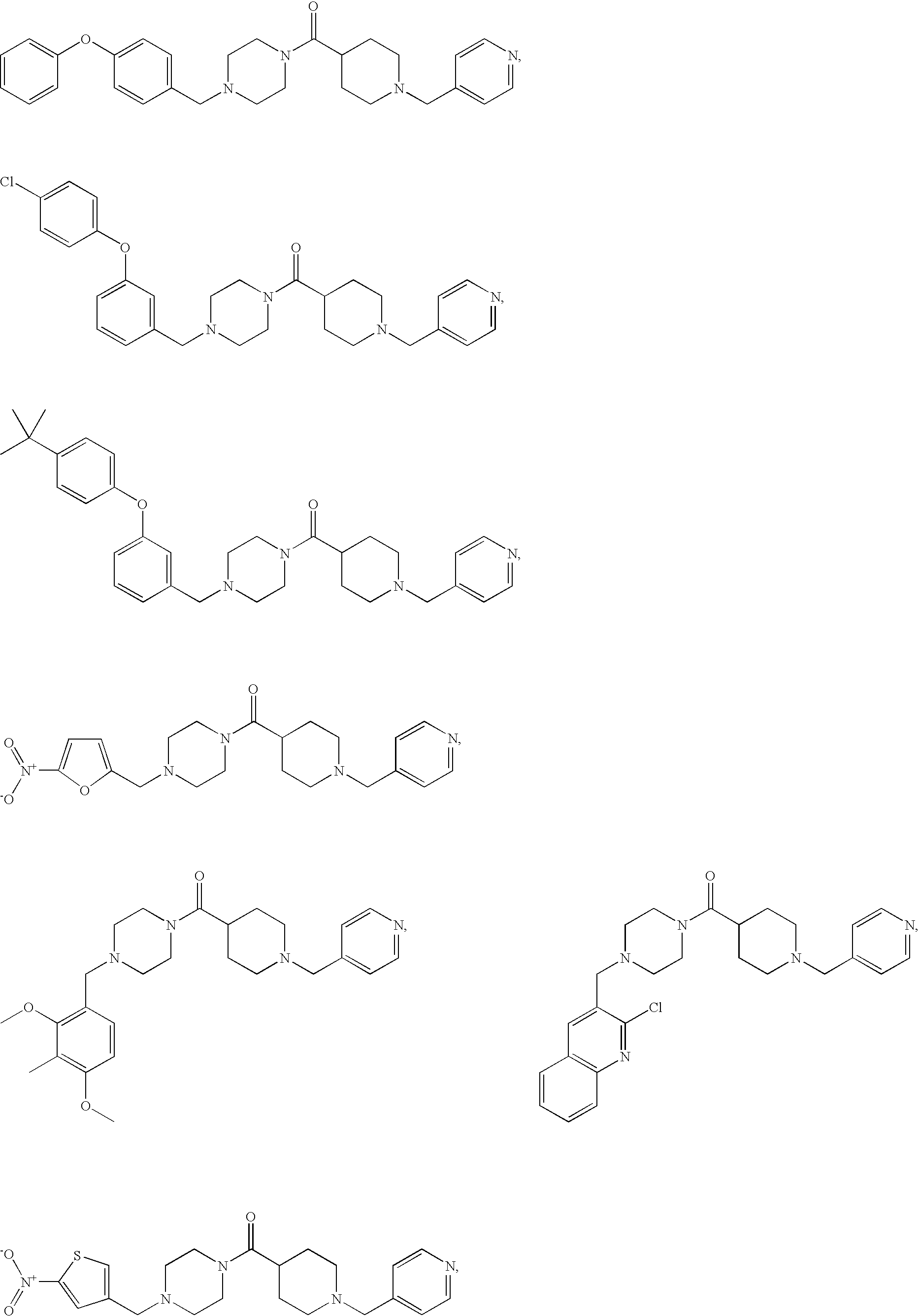

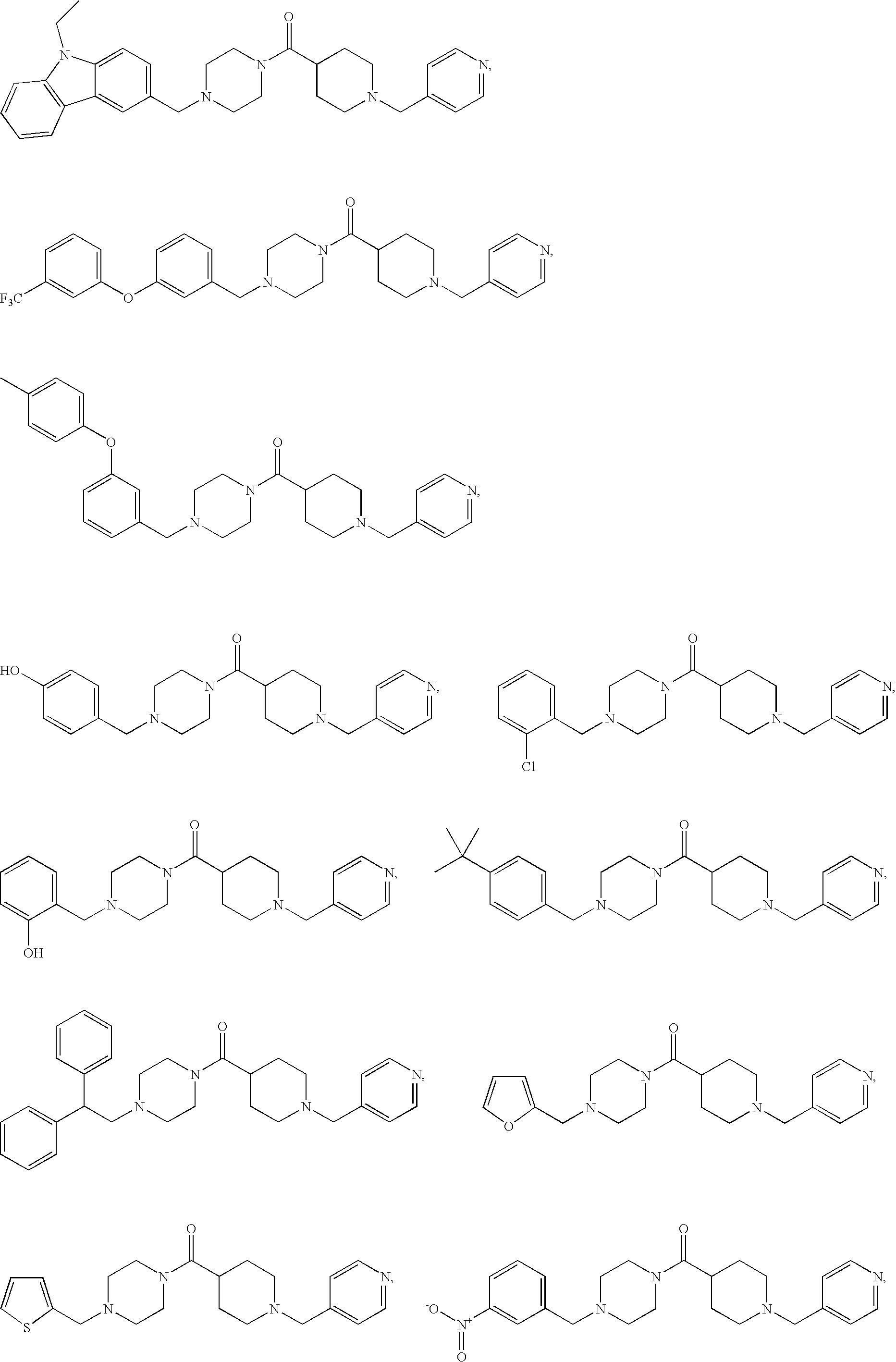

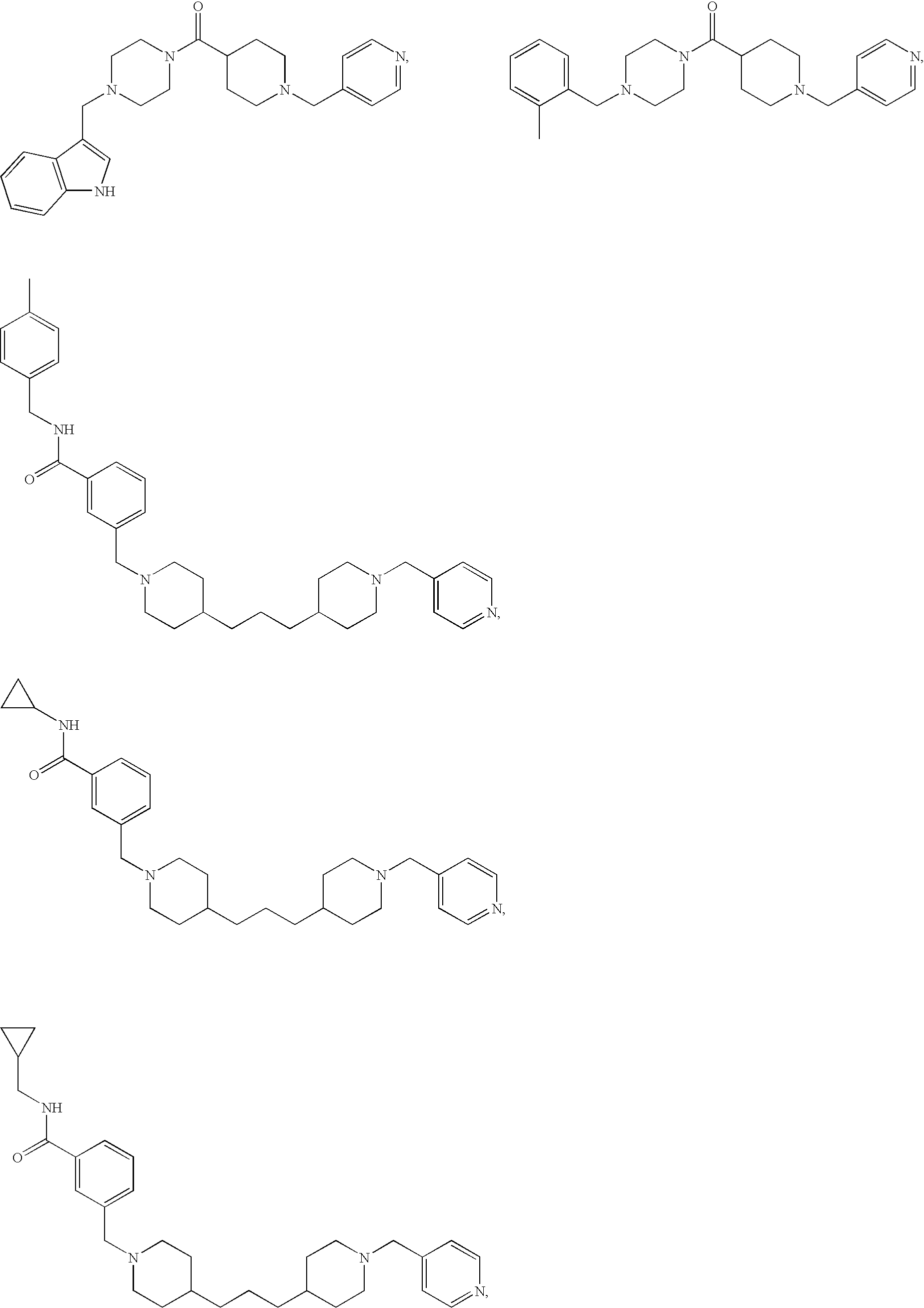

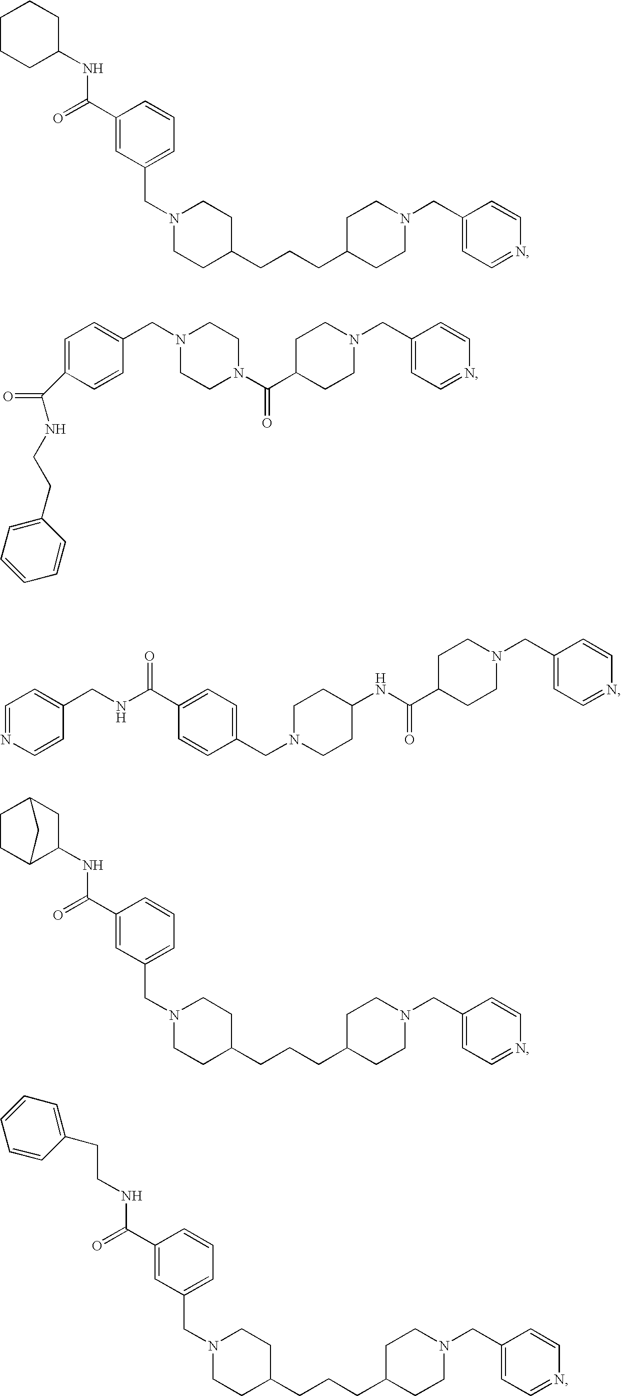

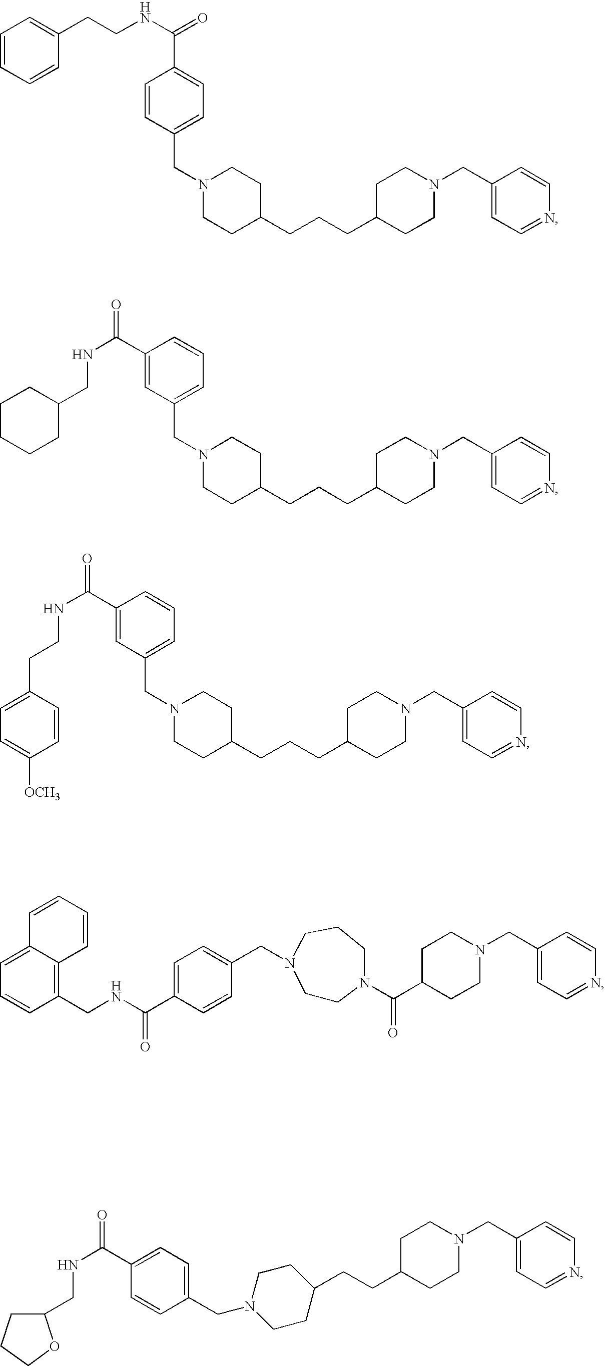

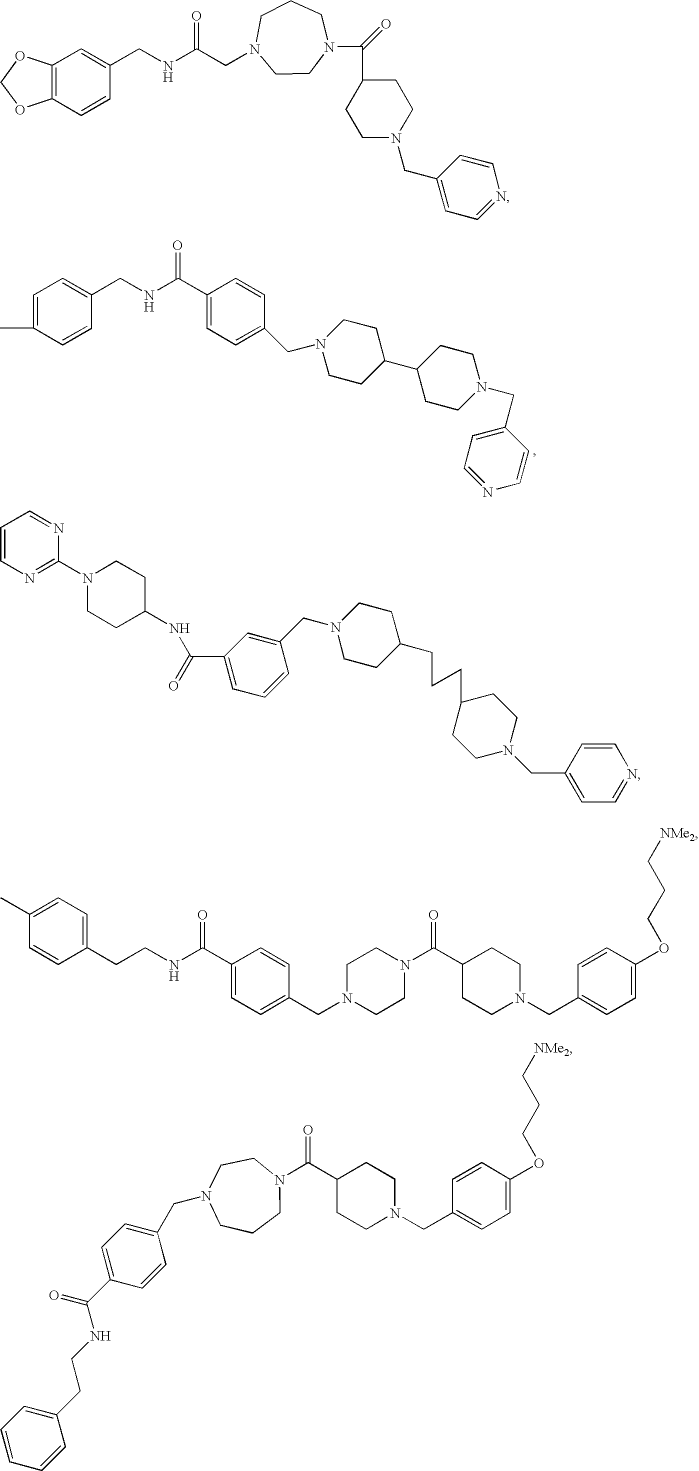

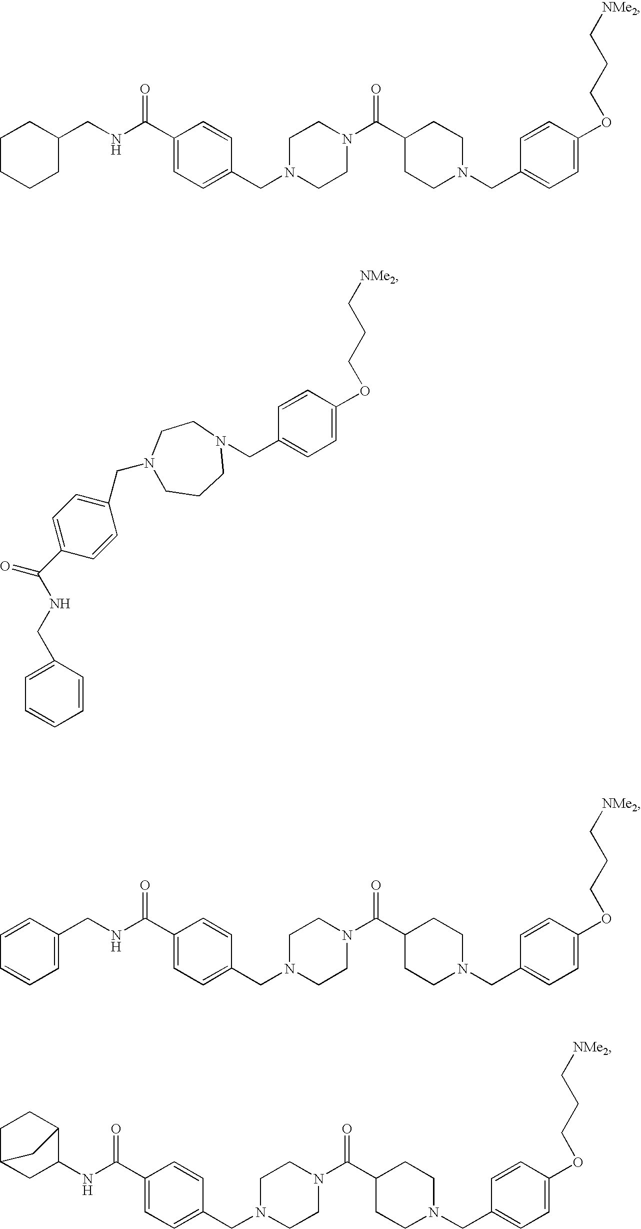

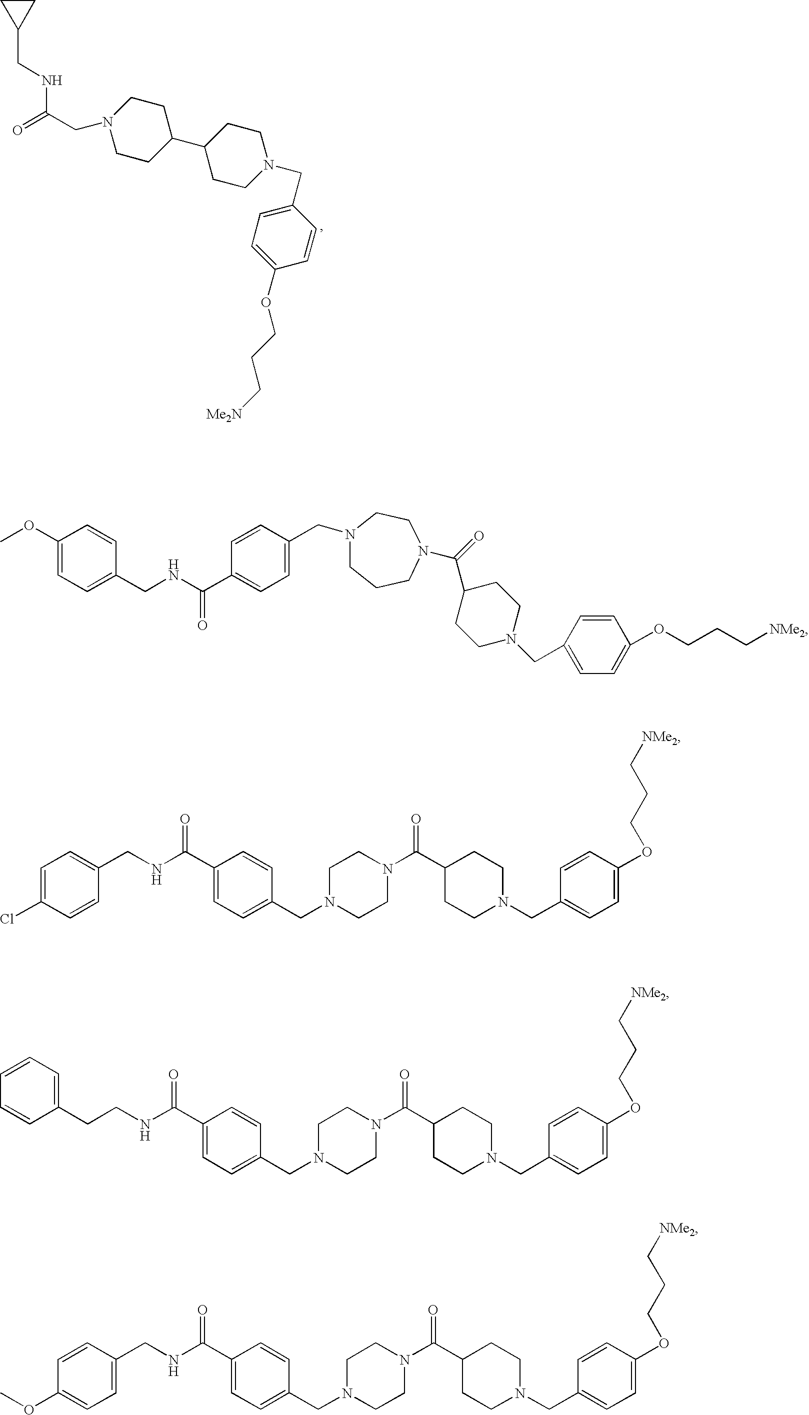

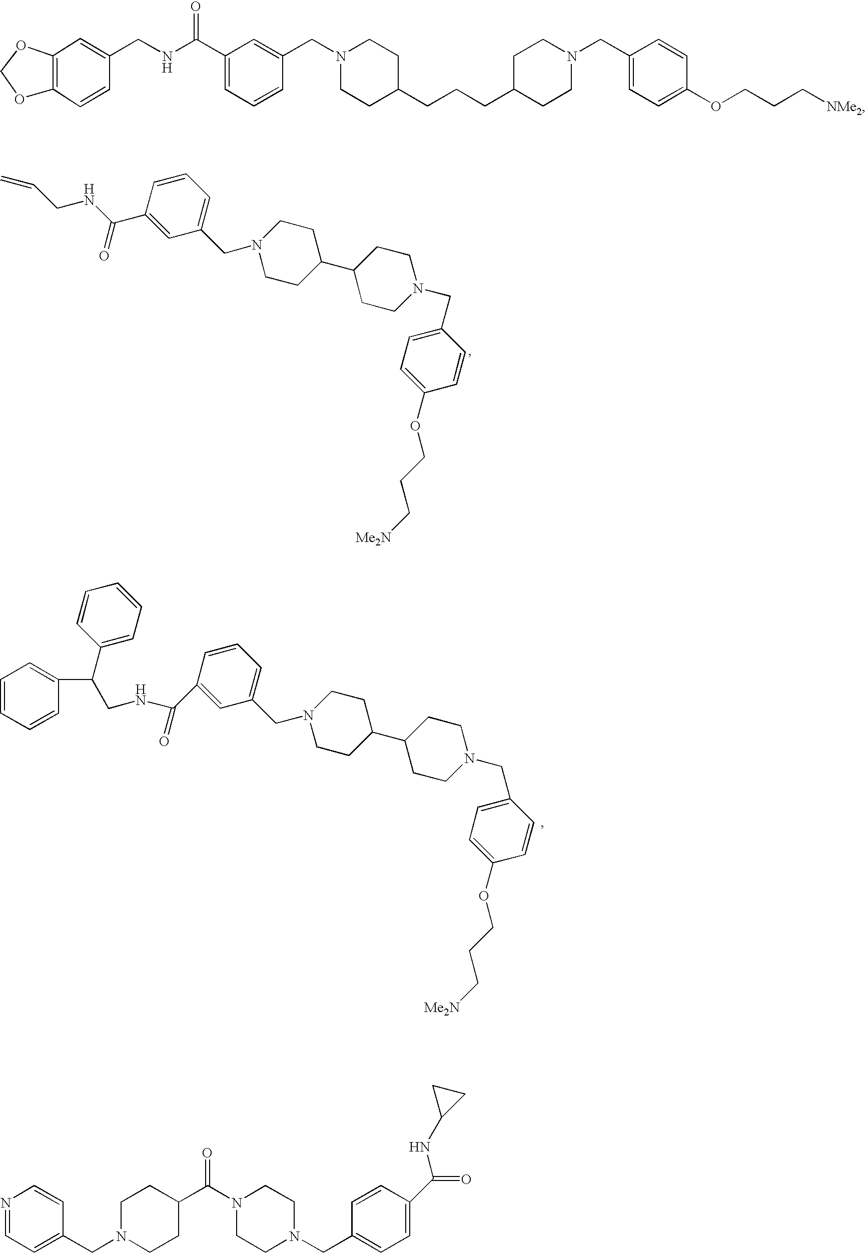

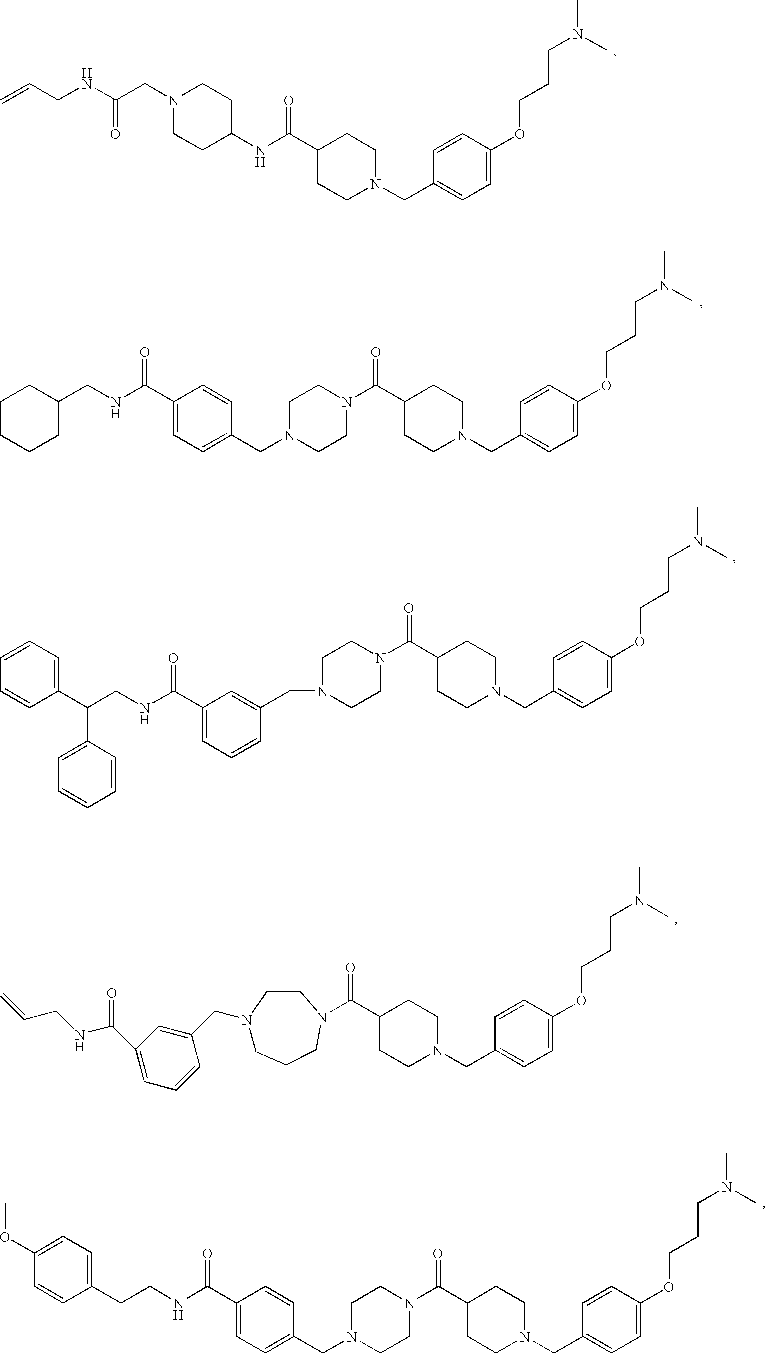

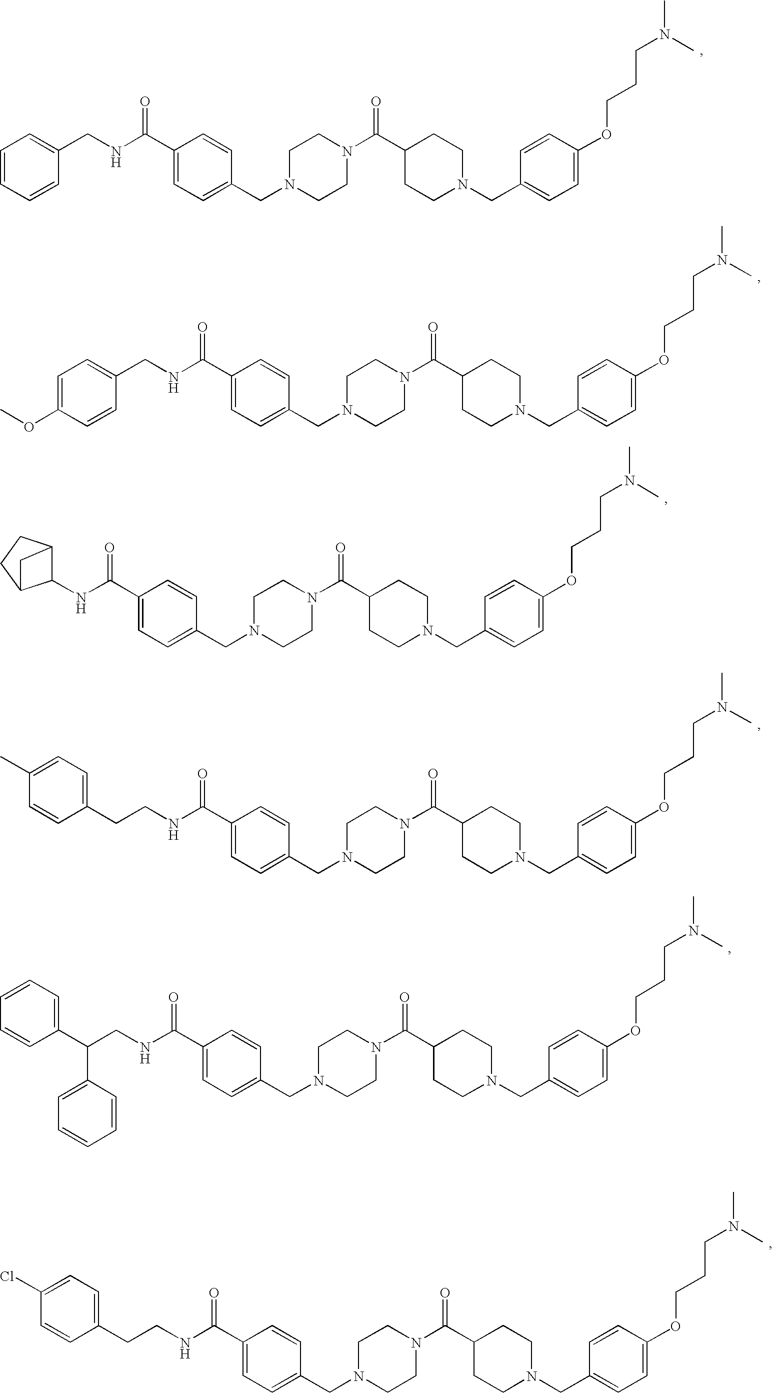

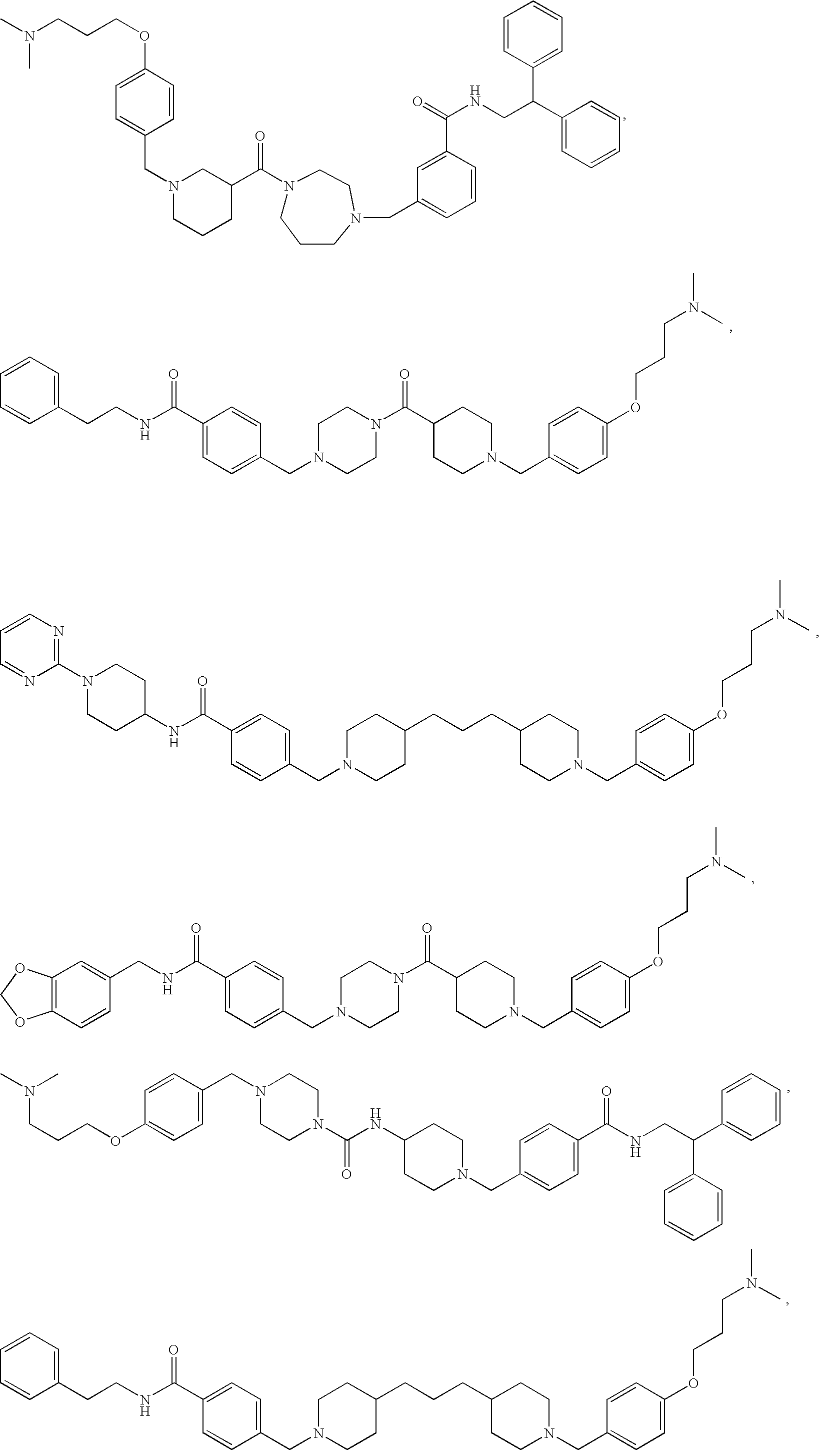

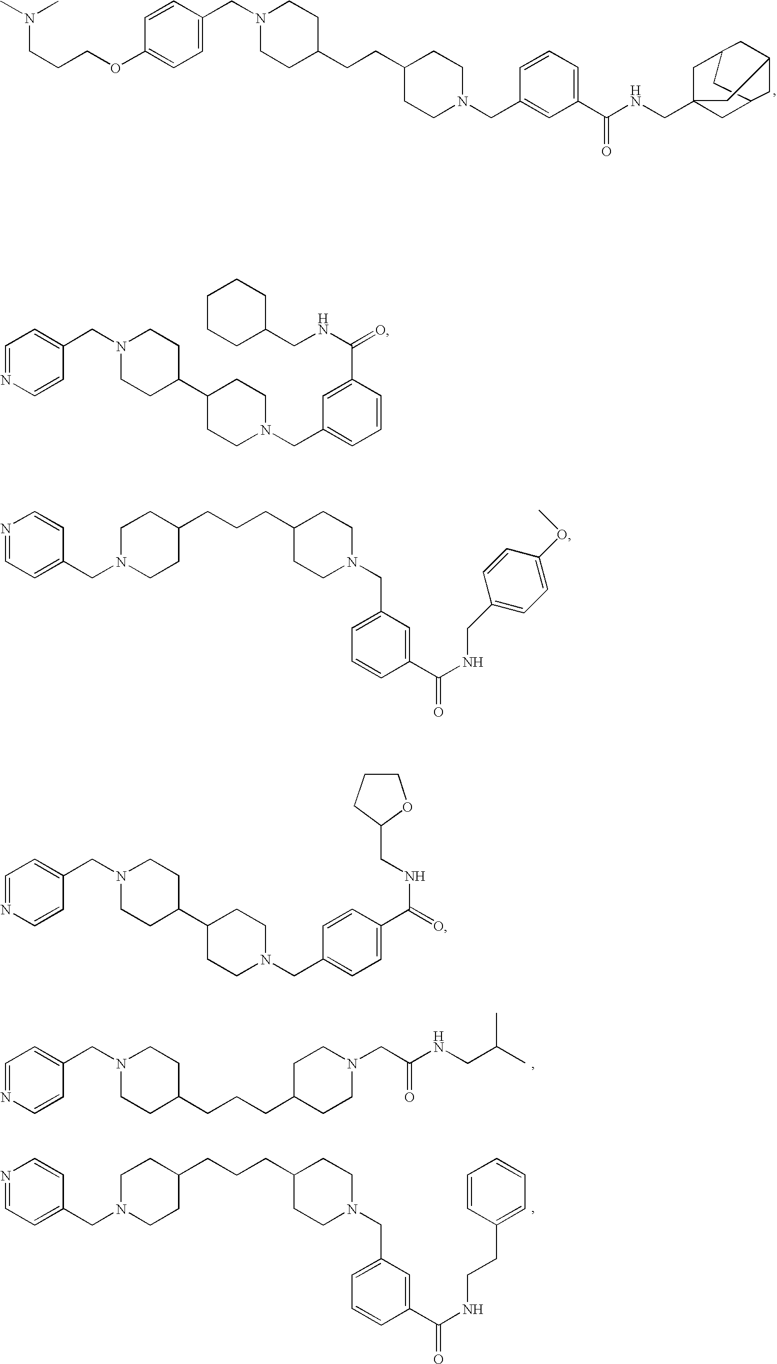

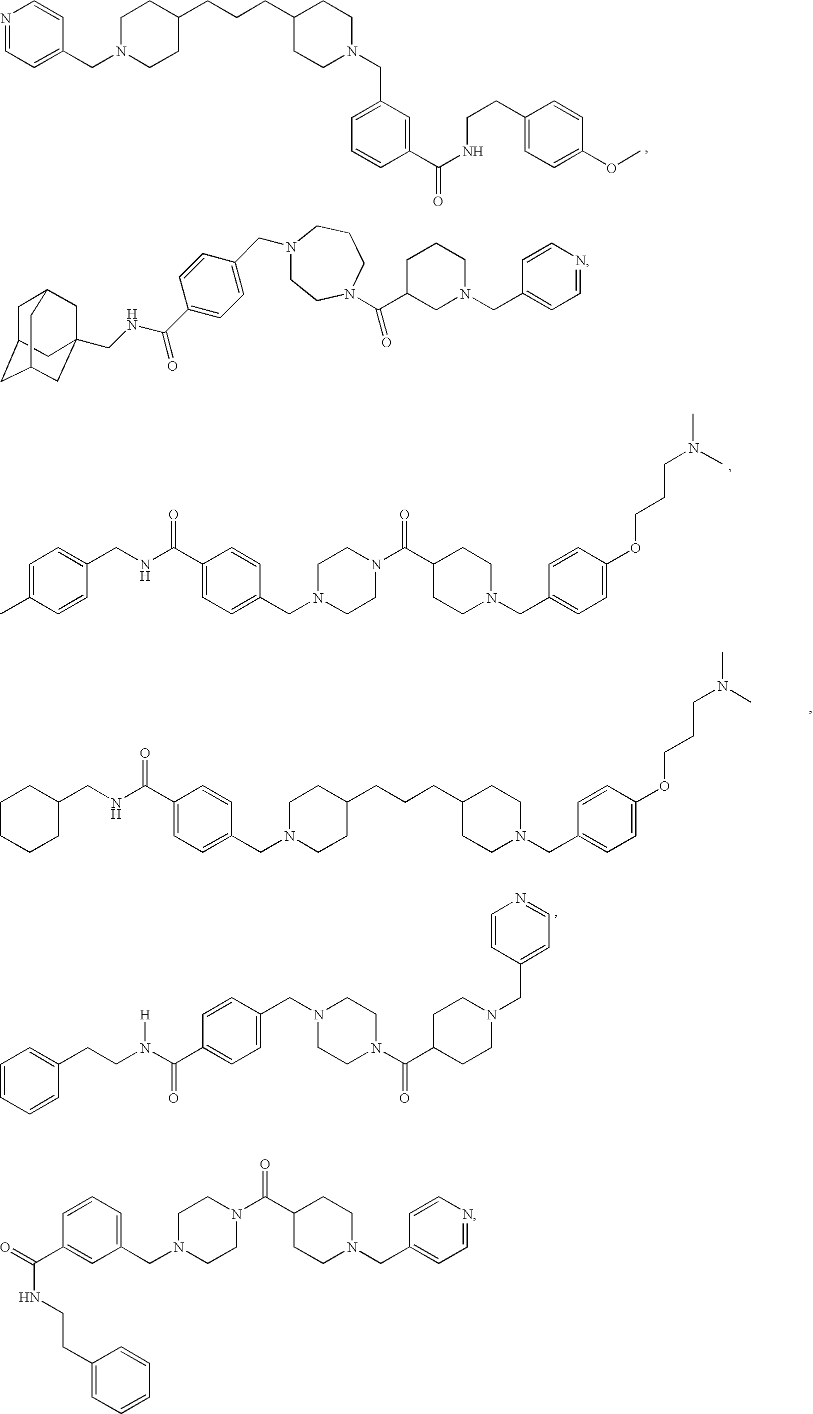

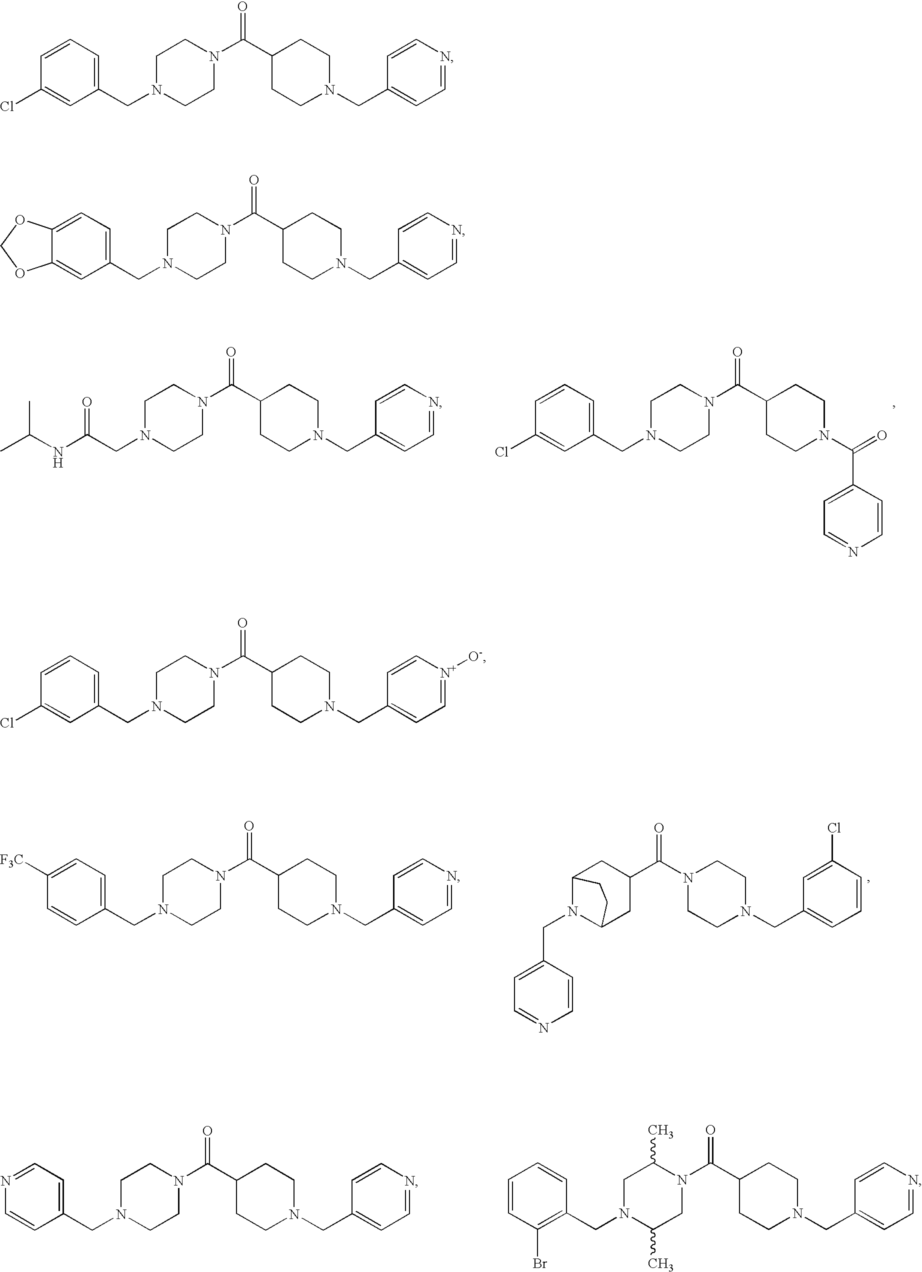

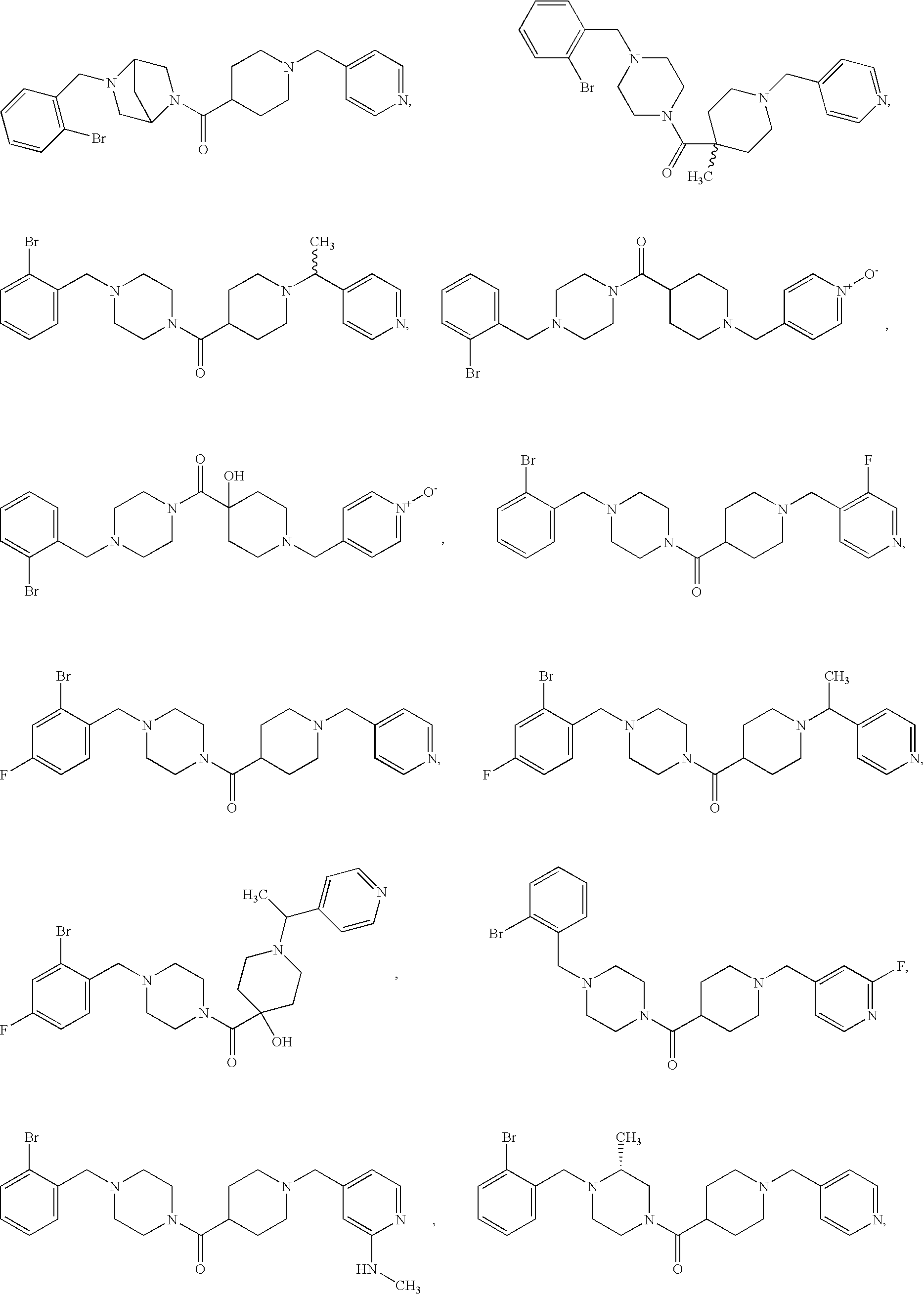

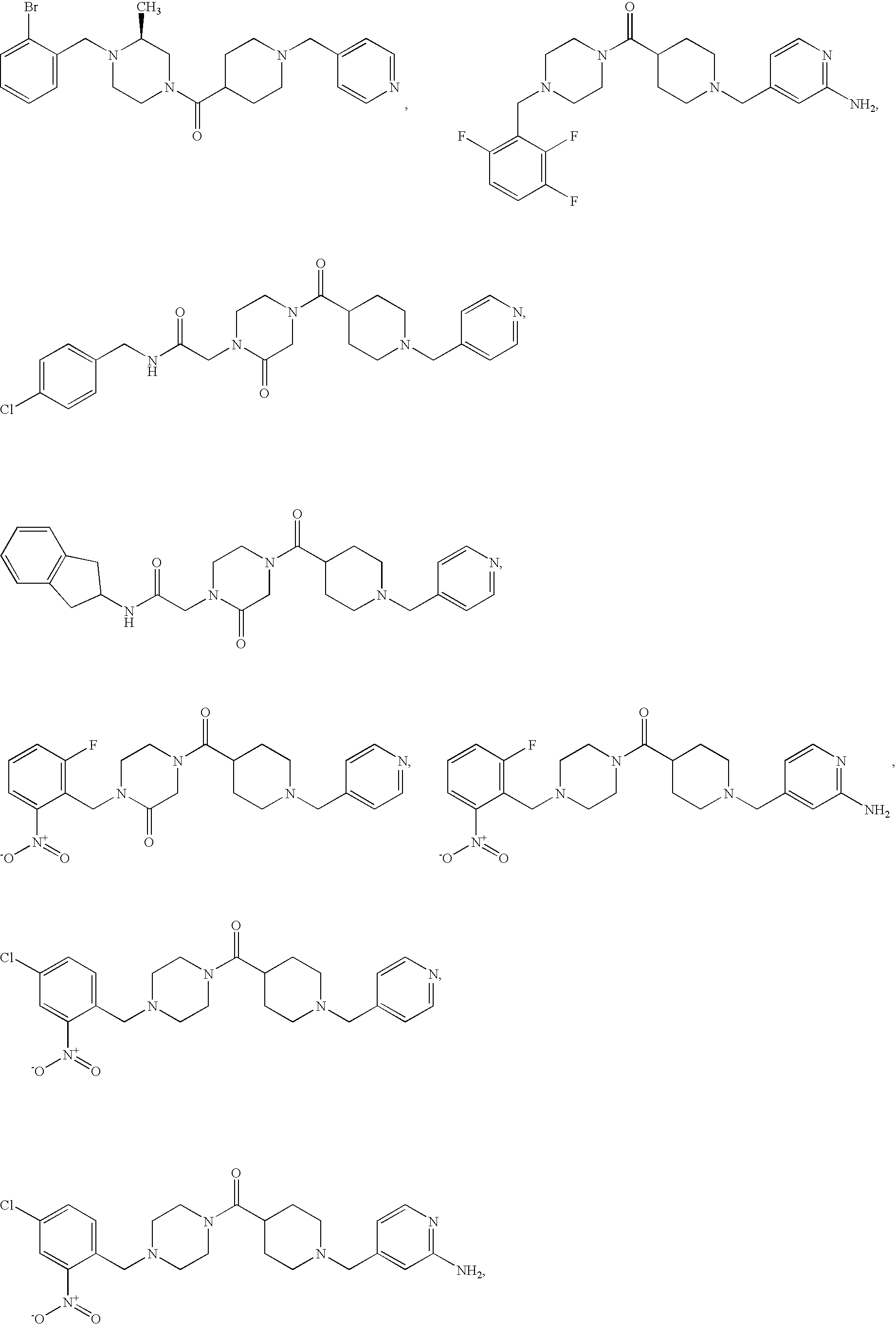

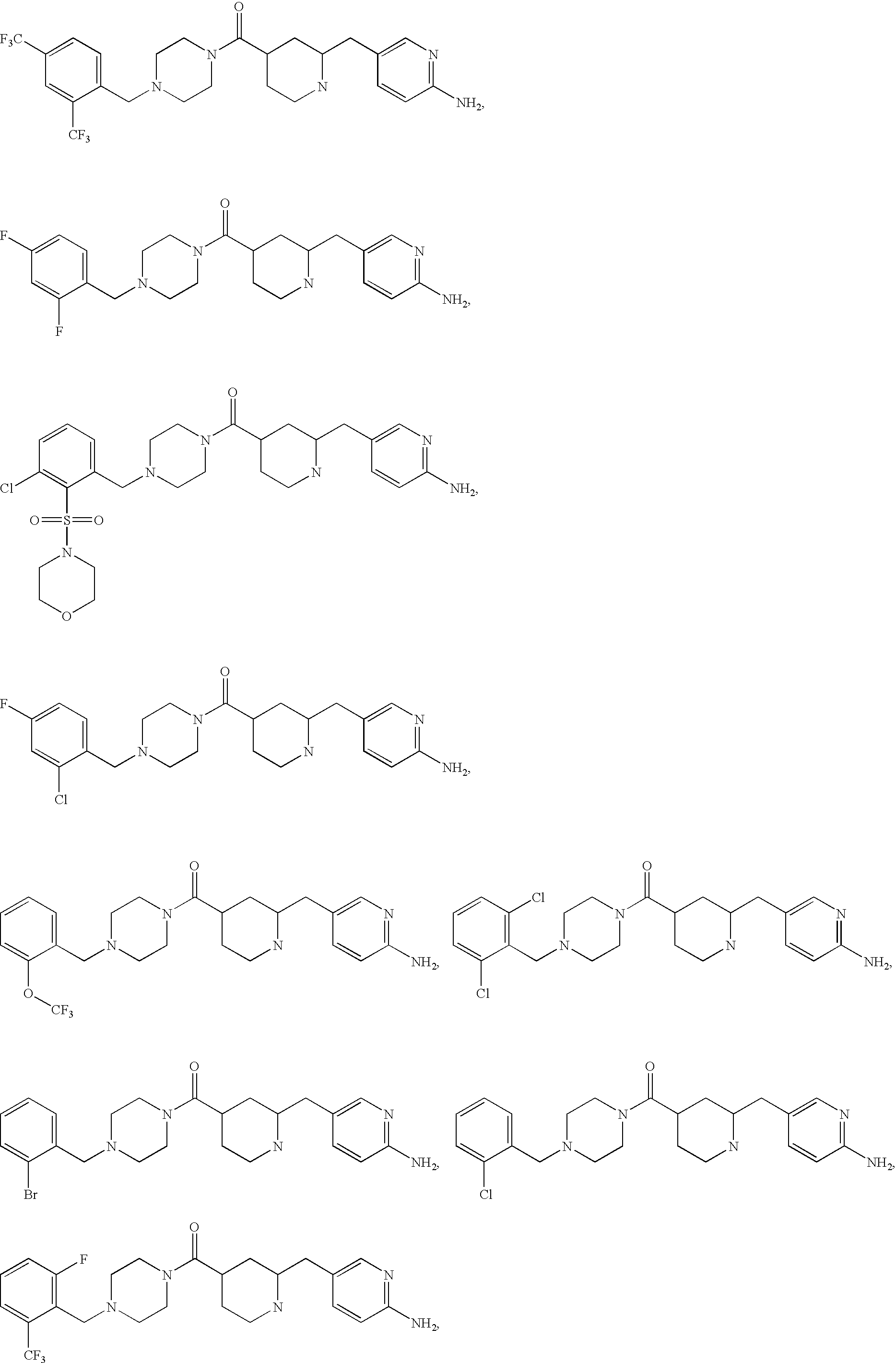

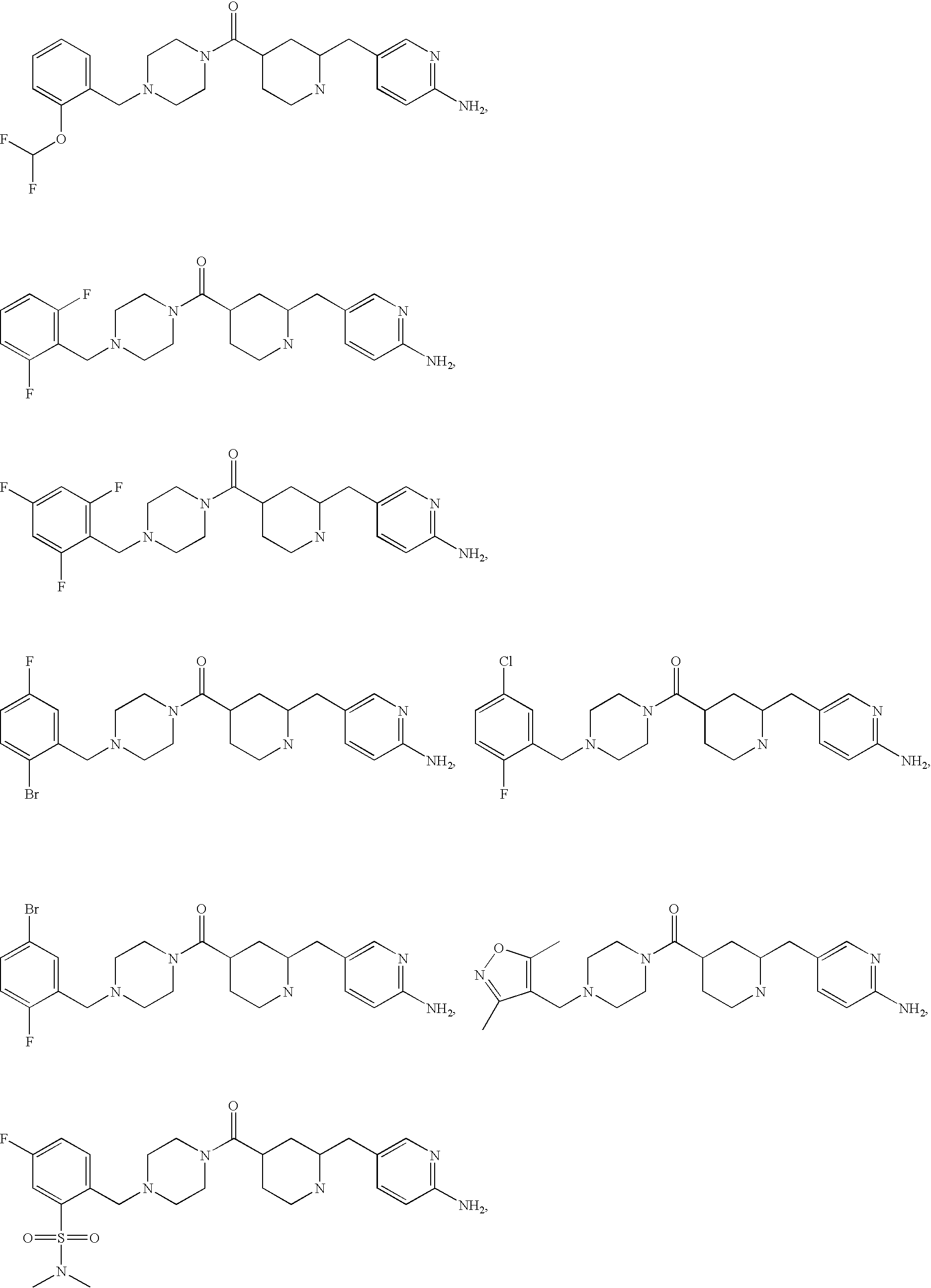

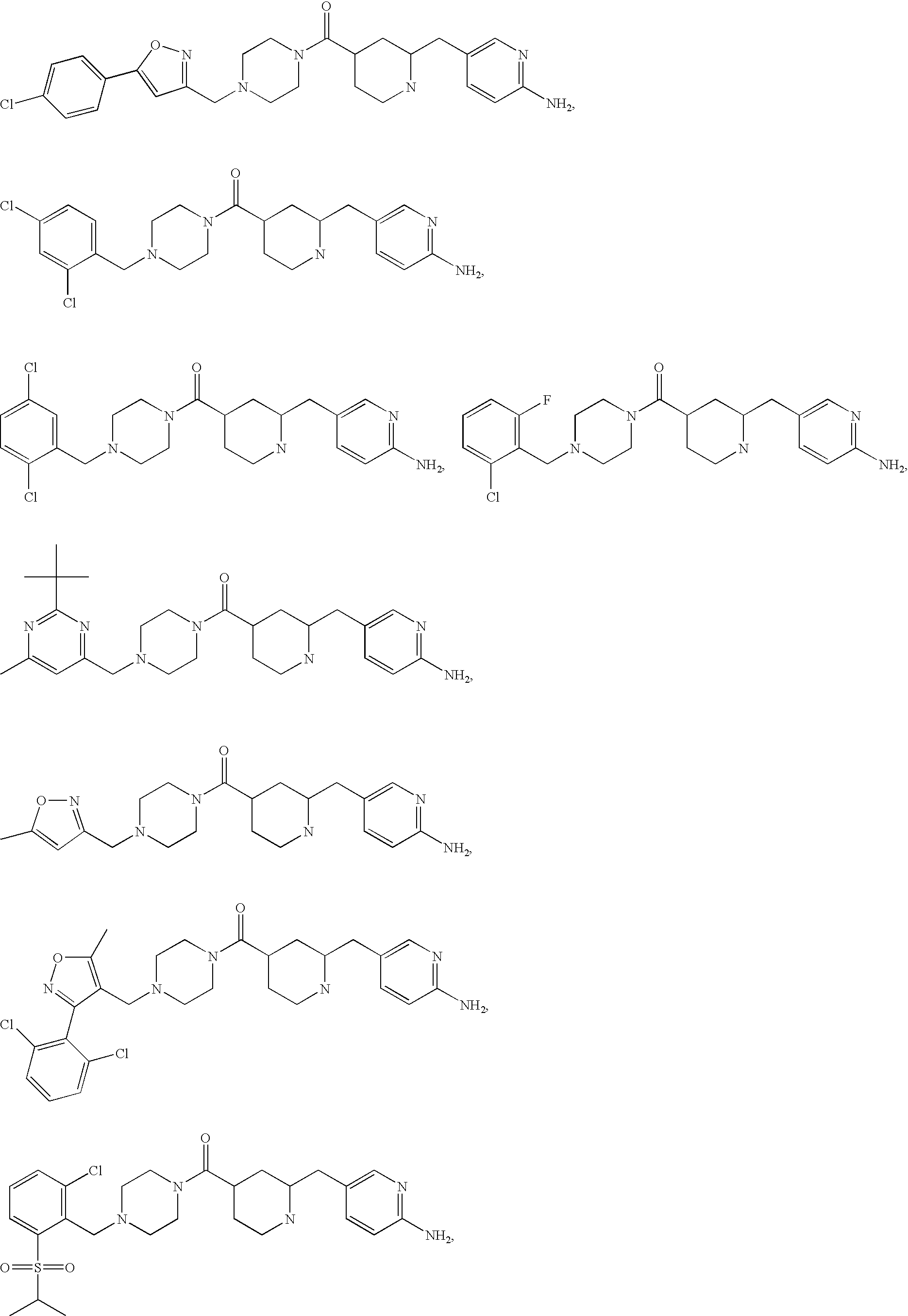

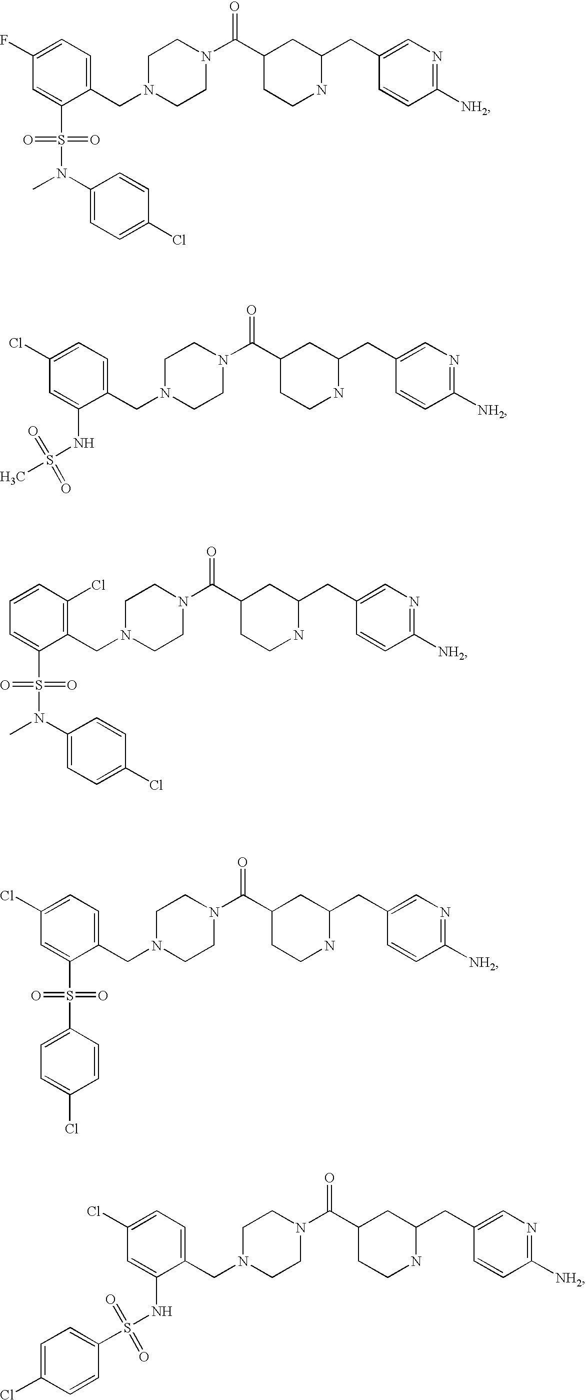

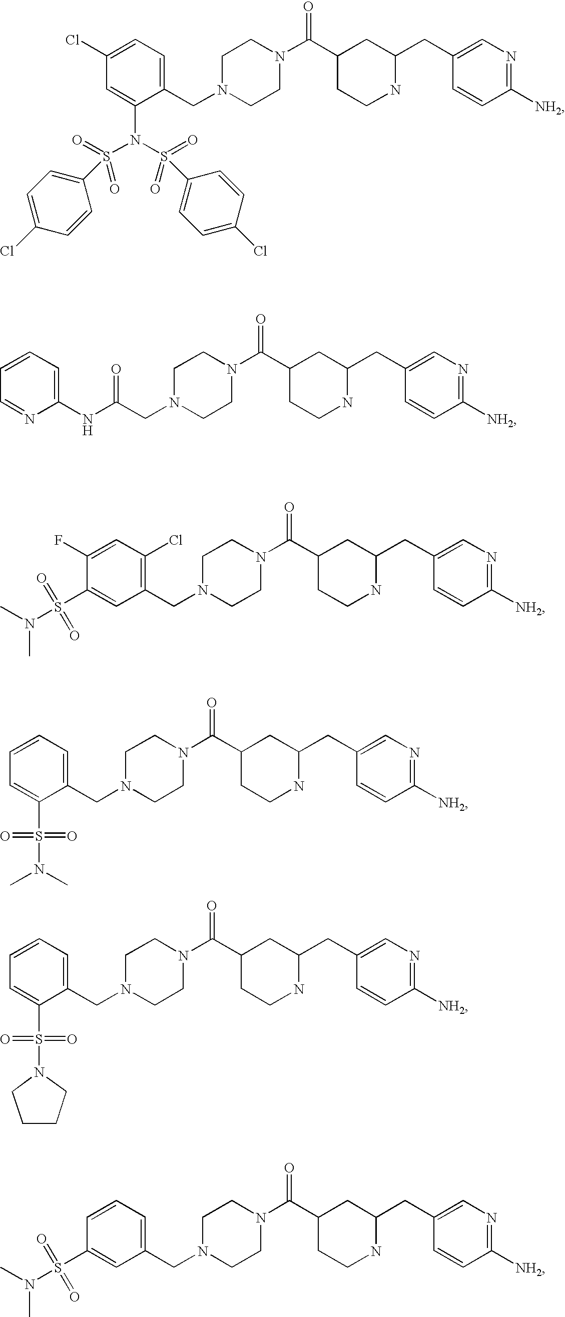

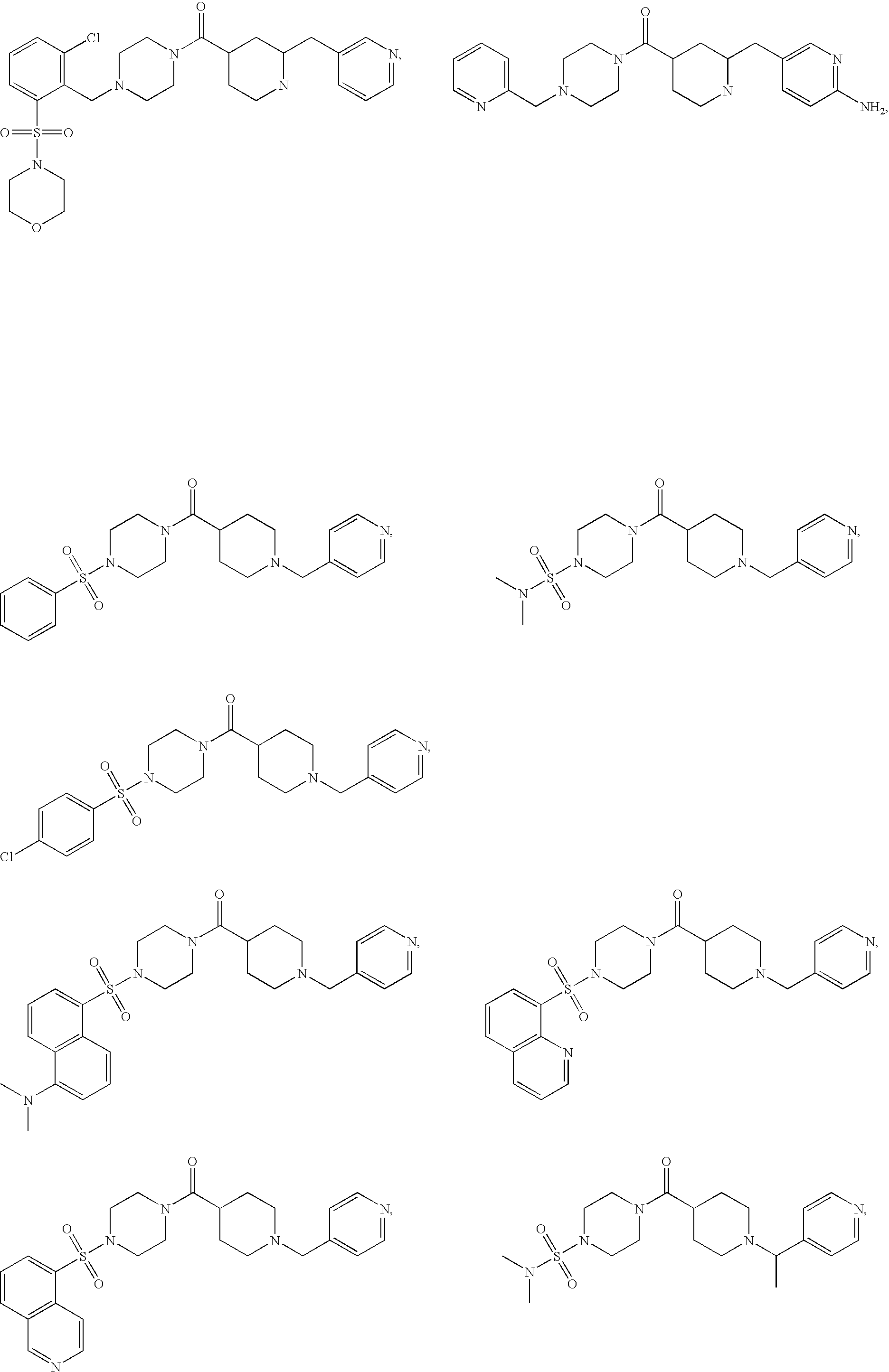

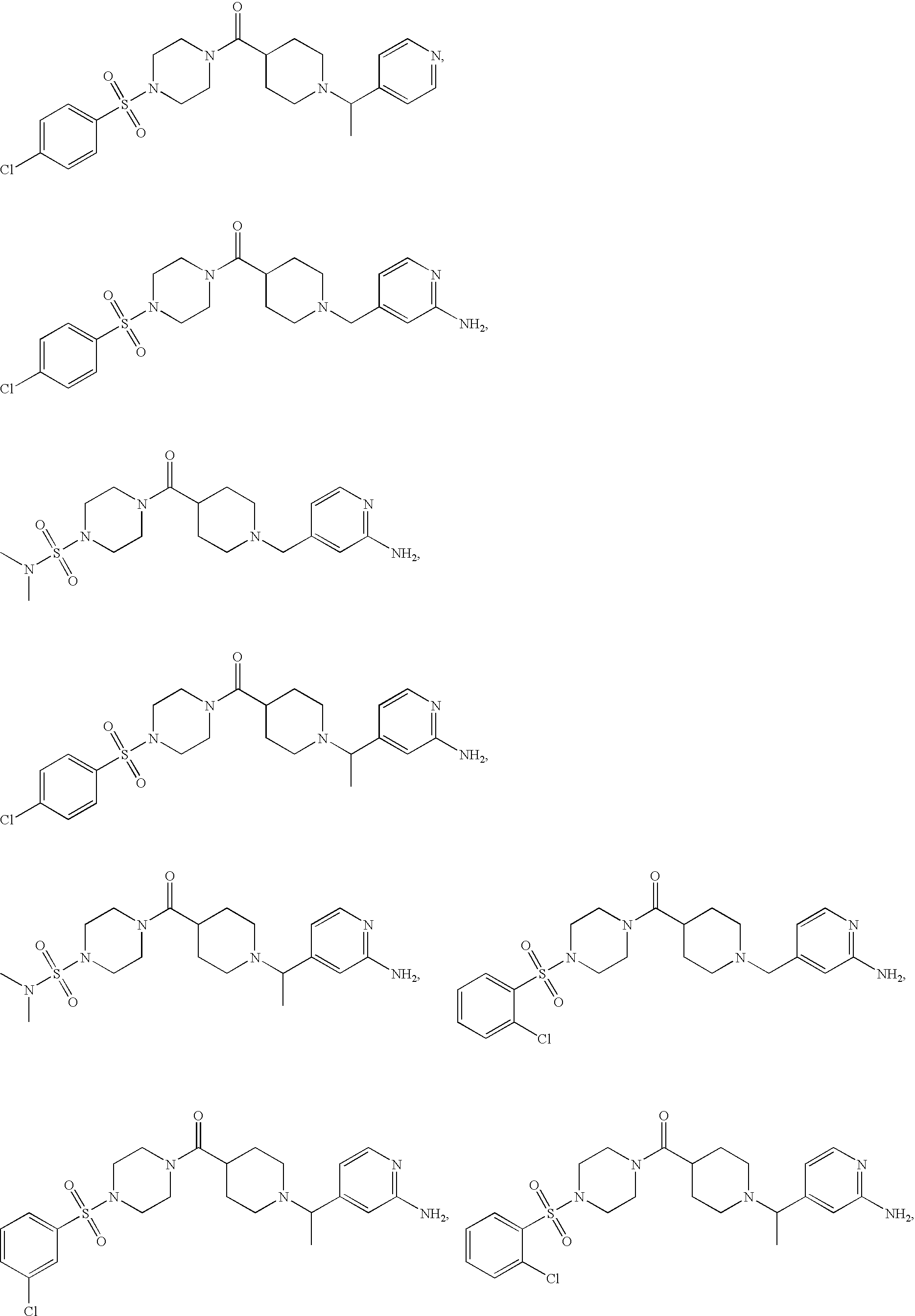

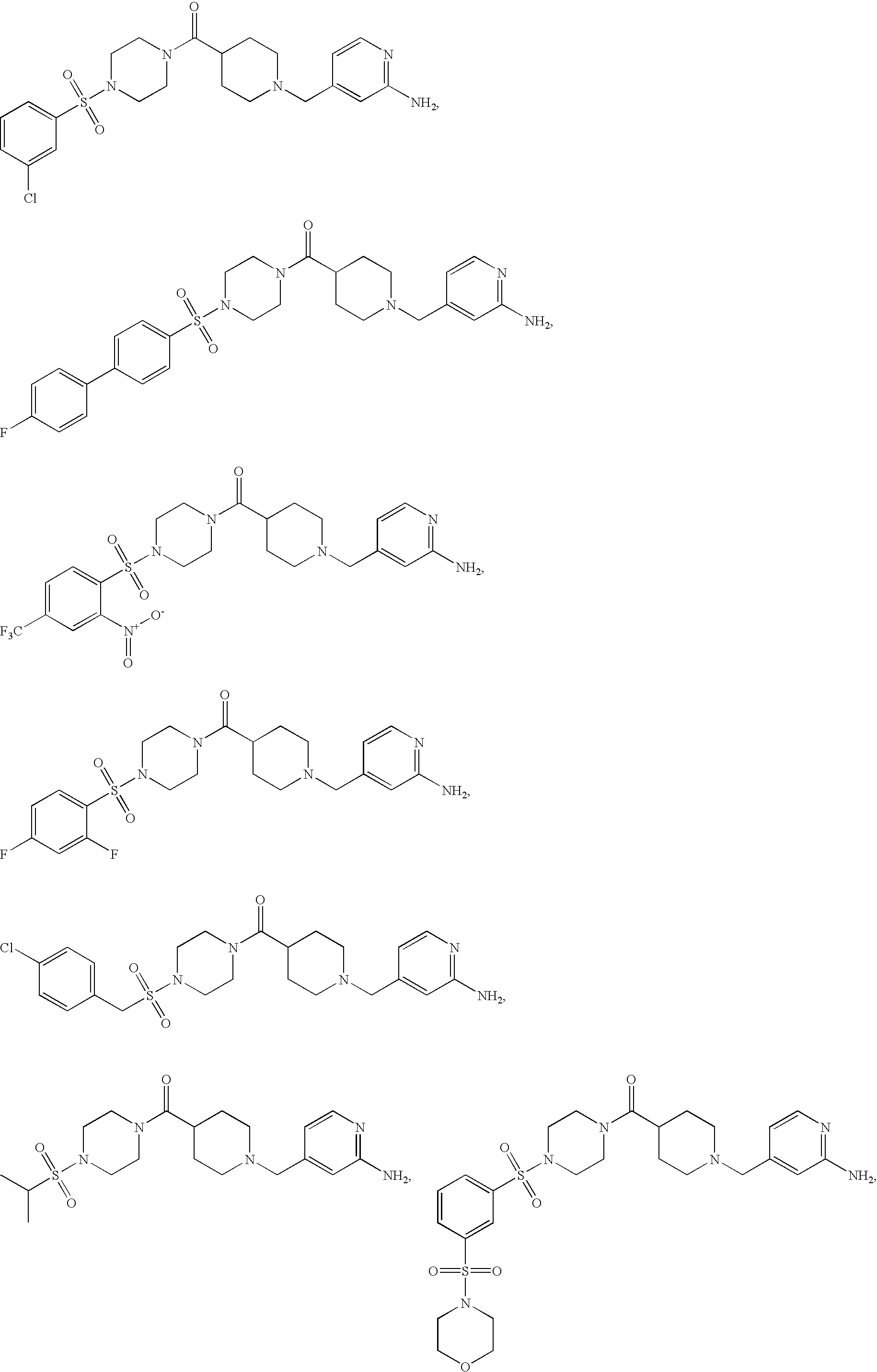

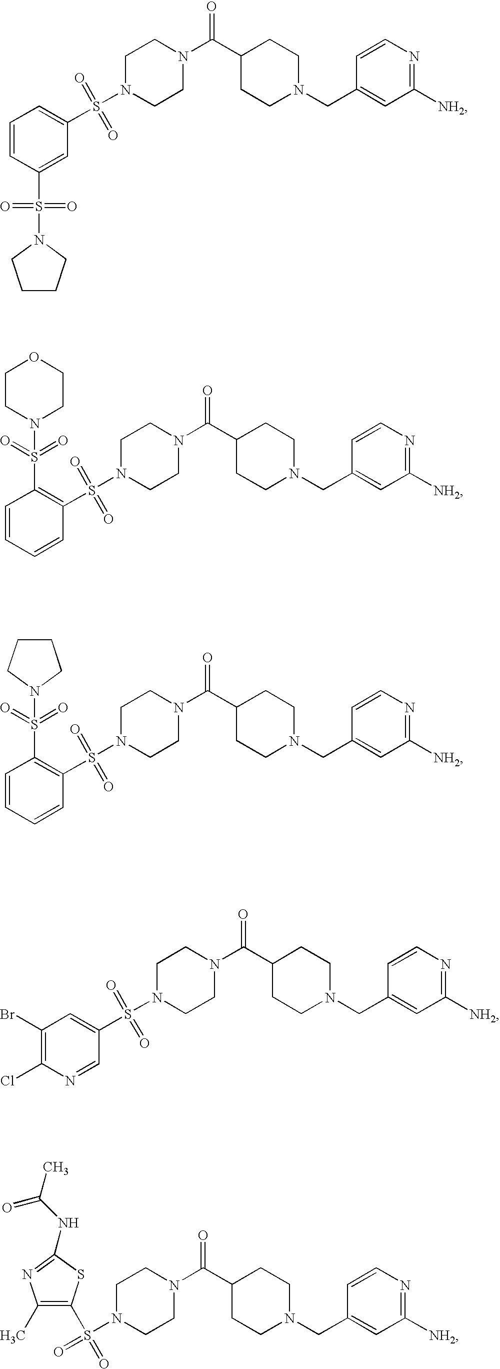

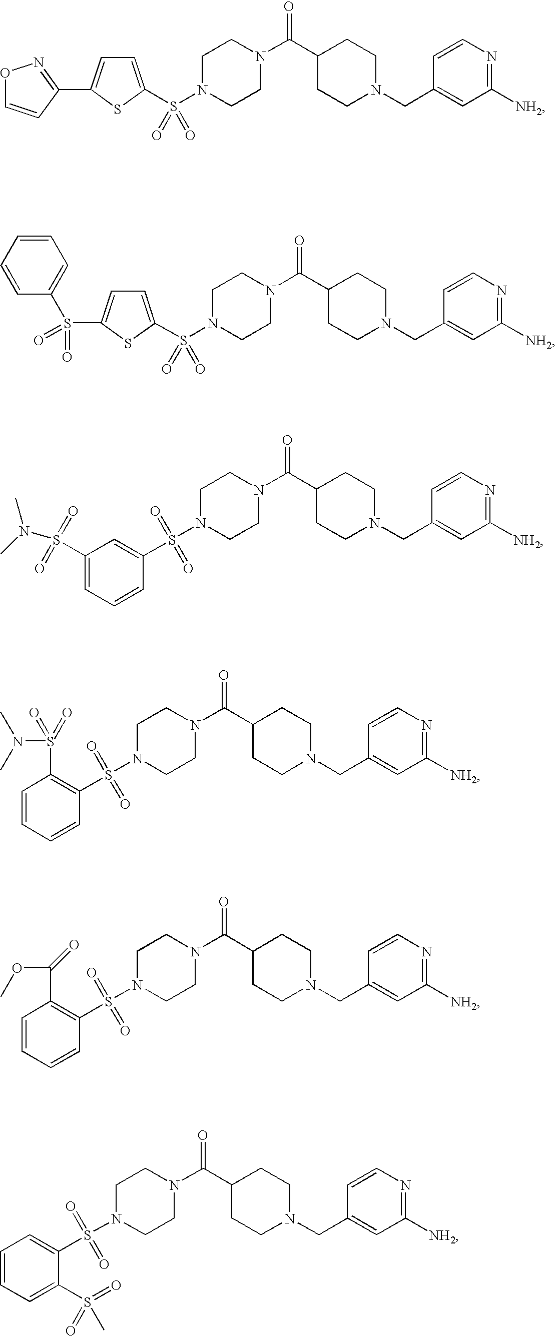

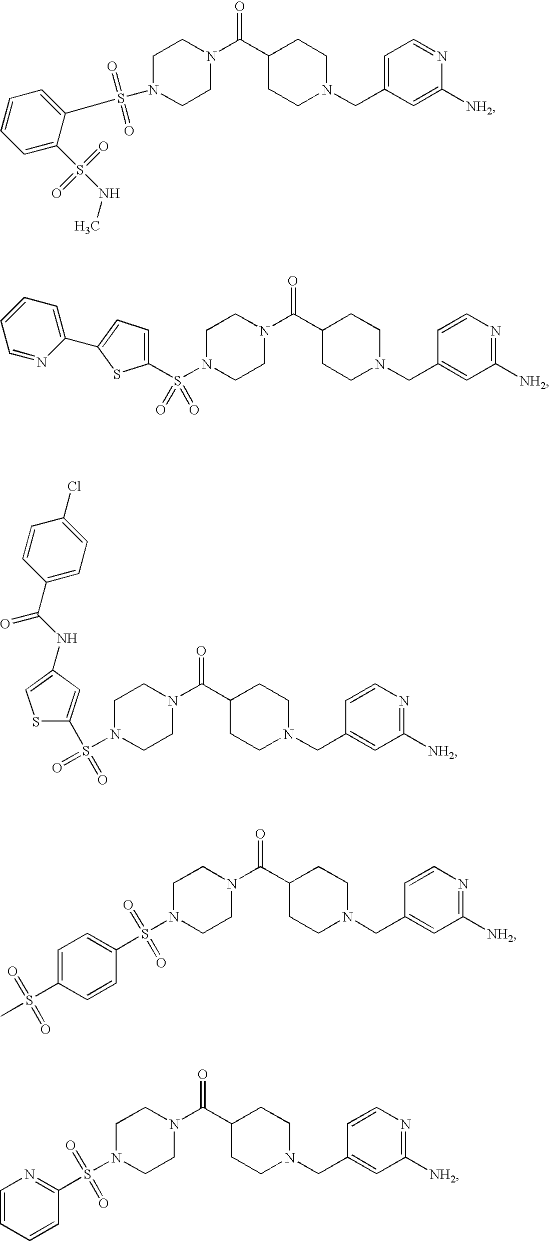

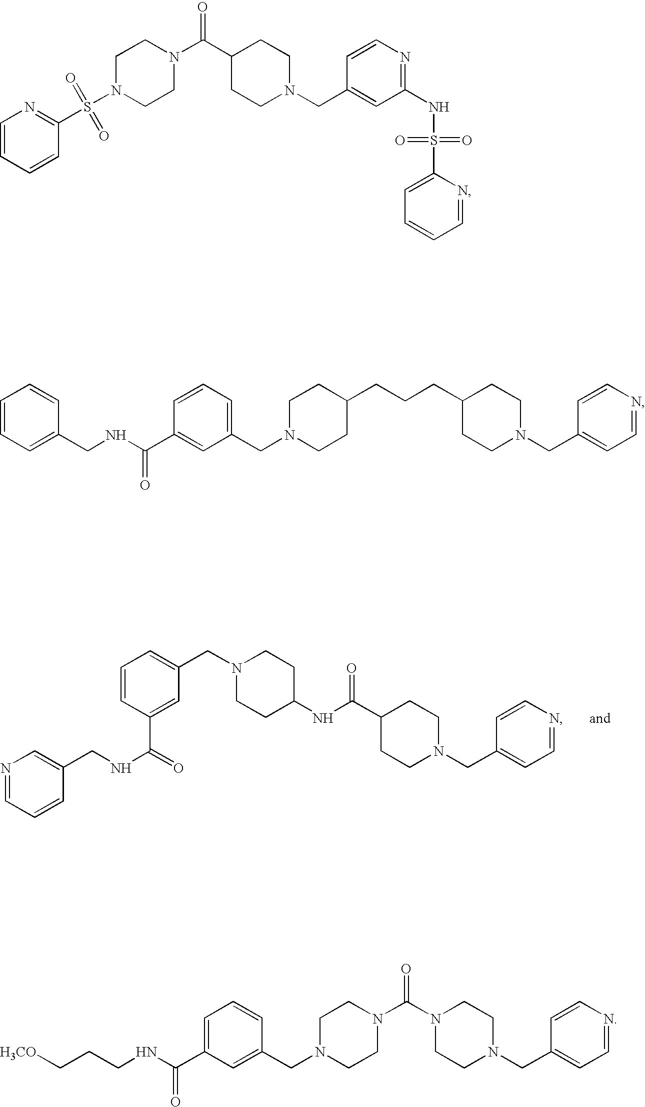

In one embodiment, the H₃ antagonists/inverse agonists of the present invention can have a structure according to Formula (III): 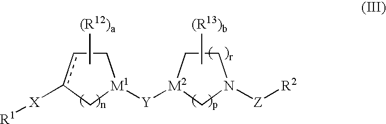
as described in U.S. Patent Publication No. 2004/0097483, which is herein incorporated by reference in its entirety. Non-limiting examples of compounds of Formula (III) include: 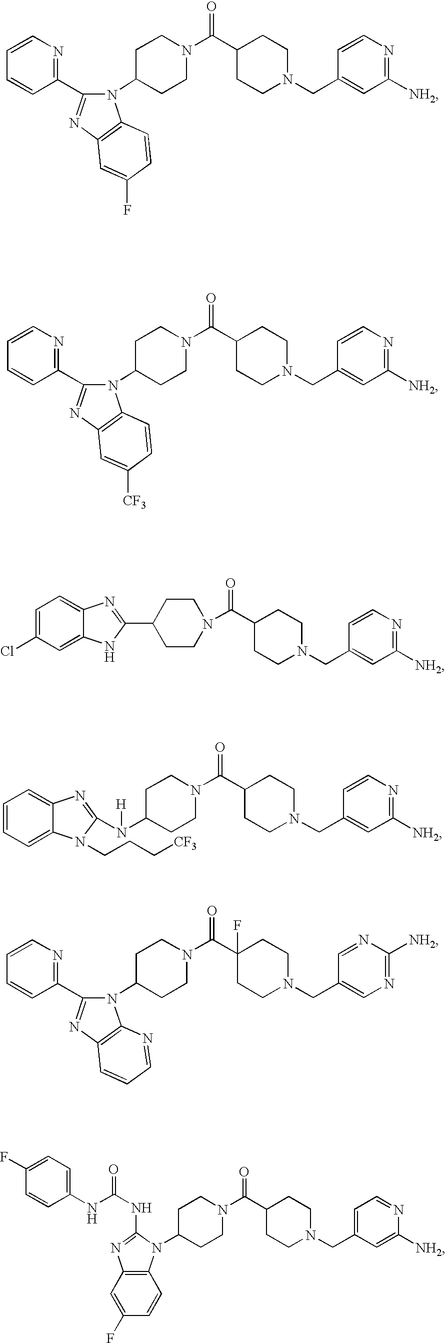
compounds of the following general formula:

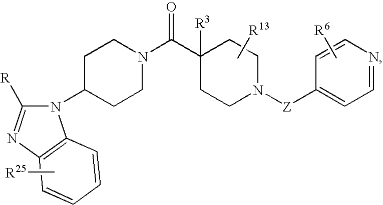

wherein R, R²⁵, R³, R¹³, Z, and R⁶ are as shown in the following Table:

| R | R²⁵ | R³ | R¹³ | Z | R⁶ |
|---|---|---|---|---|---|
| —CH₃ | 5-OCH₃ | H | H | —CH₂— | 2-NH₂ |
| —CH₃ | 6-Cl | H | H | —CH₂— | 2-NH₂ |
| —CH₃ | 5-Cl | H | H | —CH₂— | 2-NH₂ |
| —CH₃ | 5-Br | H | H | —CH₂— | 2-NH₂ |
| 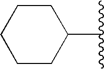 | 5-Cl | H | H | —CH₂— | 2-NH₂ |
| benzyl | 5-F | H | H | —CH₂— | 2-NH₂ |
| —CH(CH₃)₂ | 5-Br | H | H | —CH₂— | 2-NH₂ |
| —CH₂NH₂ | H | H | H | —CH₂— | 2-NH₂ |
| —CH₂NHSO₂CH₃ | H | H | H | —CH₂— | 2-NH₂ |
| —CH₂NHO(O)CH₃ | 5-Cl | H | H | —CH₂— | 2-NH₂ |
| —CH₂OCH₃ | 5-F | H | H | —CH₂— | 2-NH₂ |
| —CH₂NH₂ | 5-Cl | H | H | —CH₂— | 2-NH₂ |
| —CH₂OCH₃ | 6,7-di-F | H | H | —CH₂— | 2-NH₂ |
| 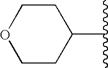 | 6-F | H | H | —CH₂— | 2-NH₂ |
| 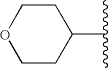 | 5-F | H | H | —CH₂— | 2-NH₂ |
| 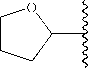 | 6-F | H | H | —CH₂— | 2-NH₂ |
| 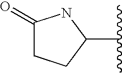 | 5-F | H | H | —CH₂— | 2-NH₂ |
| 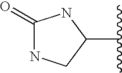 | 5-F | H | H | —CH₂— | 2-NH₂ |
| 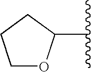 | 5-Br | H | H | —CH₂— | 2-NH₂ |
| 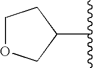 | 5-F | H | H | —CH₂— | 2-NH₂ |
| 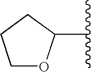 | 5-F | H | H | —CH₂— | 2-NH₂ |
| 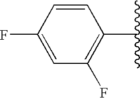 | H | H | H | —CH₂— | 2-NH₂ |
| 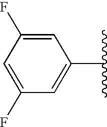 | 5-F | H | H | —CH₂— | 2-NH₂ |
| 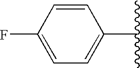 | 6-F | H | H | —CH₂— | 2-NH₂ |
| 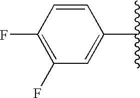 | 6,7-di-F | H | H | —CH₂— | 2-NH₂ |
| 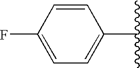 | 6-Cl | H | H | —CH₂— | 2-NH₂ |
| 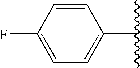 | 5-F | H | H | —CH₂— | 2-NH₂ |
| 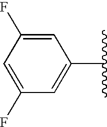 | 5-Cl | H | H | —CH₂— | 2-NH₂ |
| 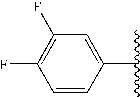 | H | H | H | —CH₂— | 2-NH₂ |
| 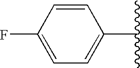 | 5-Cl | H | H | —CH₂— | 2-NH₂ |
| 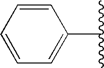 | 5-Cl | H | H | —CH₂— | 2-NH₂ |
| 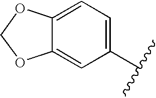 | 6-F | H | H | —CH₂— | 2-NH₂ |
| 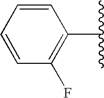 | 5-Br | H | H | —CH₂— | 2-NH₂ |
| 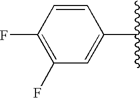 | 5-Br | H | H | —CH₂— | 2-NH₂ |
| 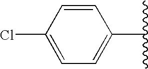 | 5-F | H | H | —CH₂— | 2-NH₂ |
| 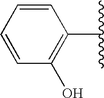 | 5-F | H | H | —CH₂— | 2-NH₂ |
| 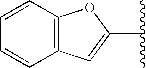 | 6-F | H | H | —CH₂— | 2-NH₂ |
| 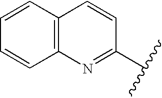 | 6-F | H | H | —CH₂— | 2-NH₂ |
| 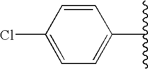 | H | H | H | —CH₂— | 2-NH₂ |
| 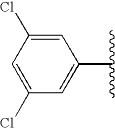 | 5-F | H | H | —CH₂— | 2-NH₂ |
| 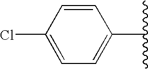 | 5-Cl | H | H | —CH₂— | 2-NH₂ |
| 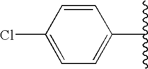 | 6-Cl | H | H | —CH₂— | 2-NH₂ |
| 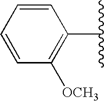 | 5-F | H | H | —CH₂— | 2-NH₂ |
| 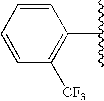 | 5-F | H | H | —CH₂— | 2-NH₂ |
| 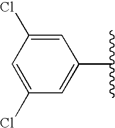 | 5-Cl | H | H | —CH₂— | 2-NH₂ |
| 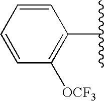 | 5-F | H | H | —CH₂— | 2-NH₂ |
| 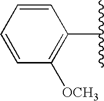 | 5-Br | H | H | —CH₂— | 2-NH₂ |
| 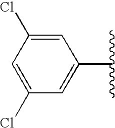 | 6-Cl | H | H | —CH₂— | 2-NH₂ |
| 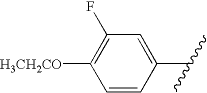 | 5-CH₃ | H | H | —CH₂— | 2-NH₂ |
| 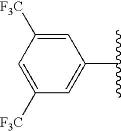 | 5-Cl | H | H | —CH₂— | 2-NH₂ |
| 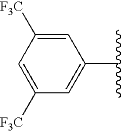 | 5-Br | H | H | —CH₂— | 2-NH₂ |
| 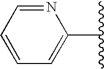 | 6-ethoxy | H | H | —CH₂— | 2-NH₂ |
| 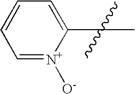 | 5-Cl | H | H | —CH₂— | 2-NH₂ |
| 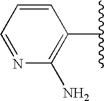 | H | H | H | —CH₂— | 2-NH₂ |
| 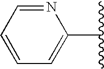 | 5-F | H | H | —CH₂— | H |
| 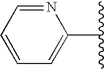 | 6-Cl | H | H | —CH₂— | 2-NH₂ |
| 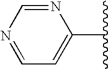 | 5-F | H | H | —CH₂— | 2-NH₂ |
| 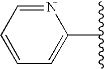 | 6-F | H | H | —CH₂— | 2-NH₂ |
| 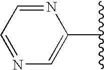 | 6-F | H | H | —CH₂— | 2-NH₂ |
| 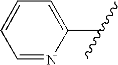 | 7-Cl | H | H | —CH₂— | 2-NH₂ |
| 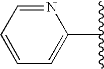 | H | H | H | —CH₂— | 2-NH₂ |
| 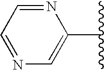 | 5-F | H | H | —CH₂— | 2-NH₂ |
| 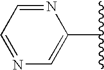 | 5-Cl | H | H | —CH₂— | 2-NH₂ |
| 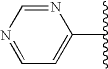 | 5-Cl | H | H | —CH₂— | 2-NH₂ |
| 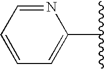 | 5,6-di-F | H | H | —CH₂— | 2-NH₂ |
| 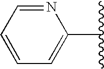 | 5-Br | H | H | —CH₂— | 2-NH₂ |
| 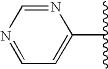 | 6-ethoxy | H | H | —CH₂— | 2-NH₂ |
| 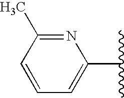 | 5-F | H | H | —CH₂— | 2-NH₂ |
| 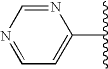 | 6-F | H | H | —CH₂— | 2-NH₂ |
| 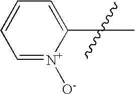 | 5-Br | H | H | —CH₂— | 2-NH₂ |
| 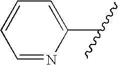 | 5-Cl | H | H | —CH₂— | 2-NH₂ |
| 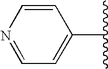 | 5-Cl | H | H | —CH₂— | 2-NH₂ |
| 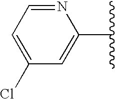 | 5-F | H | H | —CH₂— | 2-NH₂ |
| 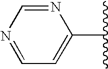 | 5-CF₃ | H | H | —CH₂— | 2-NH₂ |
| 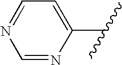 | H | H | H | —CH₂— | 2-NH₂ |
| 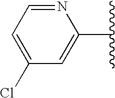 | 6,7-di-F | H | H | —CH₂— | 2-NH₂ |
| 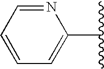 | 6,7-di-F | H | H | —CH₂— | 2-NH₂ |
| 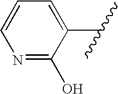 | 5-F | H | H | —CH₂— | 2-NH₂ |
| 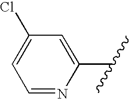 | 5-CF₃,7-F | H | H | —CH₂— | 2-NH₂ |
| 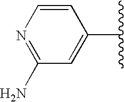 | 5-F | H | H | —CH₂— | 2-NH₂ |
| 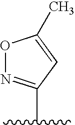 | H | H | H | —CH₂— | 2-NH₂ |
| 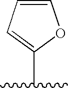 | H | H | H | —CH₂— | 2-NH₂ |
| 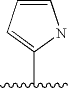 | H | H | H | —CH₂— | 2-NH₂ |
| 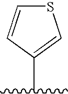 | 6-F | H | H | —CH₂— | 2-NH₂ |
| 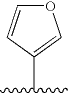 | 6-F | H | H | —CH₂— | 2-NH₂ |
| 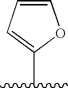 | 5-F | H | H | —CH₂— | 2-NH₂ |
| 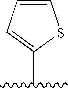 | H | H | H | —CH₂— | 2-NH₂ |
| 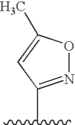 | 5-F | H | H | —CH₂— | 2-NH₂ |
| 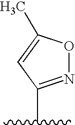 | 5-Cl | H | H | —CH₂— | 2-NH₂ |
| 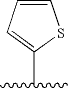 | 5-F | H | H | —CH₂— | 2-NH₂ |
| 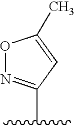 | 6,7-di-F | H | H | —CH₂— | 2-NH₂ |
| 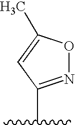 | 5-Br | H | H | —CH₂— | 2-NH₂ |
| 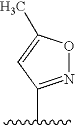 | 6-ethoxy | H | H | —CH₂— | 2-NH₂ |
| 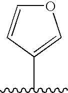 | 5-F | H | H | —CH₂— | 2-NH₂ |
| 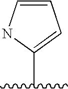 | 5-Br | H | H | —CH₂— | 2-NH₂ |
| 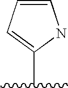 | 5-F | H | H | —CH₂— | 2-NH₂ |
| 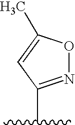 | 5-CF₃ | H | H | —CH₂— | 2-NH₂ |
| 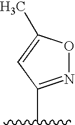 | 5-CF₃,7-F | H | H | —CH₂— | 2-NH₂ |
| 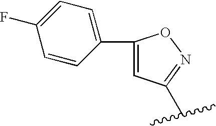 | 5-F | H | H | —CH₂— | 2-NH₂ |
| 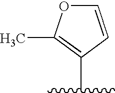 | 5-F | H | H | —CH₂— | 2-NH₂ |
| 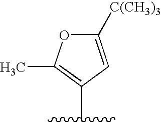 | 5-F | H | H | —CH₂— | 2-NH₂ |
| 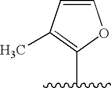 | 5-F | H | H | —CH₂— | 2-NH₂ |
| CH₃—S— | 5-F | H | H | —CH₂— | 2-NH₂ |
| CH₃—CH₂—S— | 5-F | H | H | —CH₂— | 2-NH₂ |
| CH₃—SO₂— | 5-F | H | H | —CH₂— | 2-NH₂ |
| 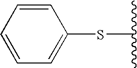 | 5-F | H | H | —CH₂— | 2-NH₂ |
| 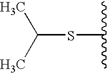 | 5-F | H | H | —CH₂— | 2-NH₂ |
| 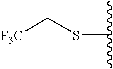 | 5-F | H | H | —CH₂— | 2-NH₂ |
| 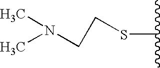 | 5-F | H | H | —CH₂— | 2-NH₂ |
| HS— | 5-F | H | H | —CH₂— | 2-NH₂ |
| CH₃—S— | 5-F | H | 2-CH₃ | —CH₂— | 2-NH₂ |
| CH₃—S— | 5-F | F | H | —CH₂— | 2-NH₂ |
| 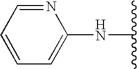 | 5-F | H | H | —CH₂— | 2-NH₂ |
| 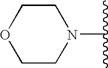 | 5-F | H | H | —CH₂— | 2-NH₂ |
| 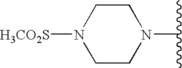 | 5-F | H | H | —CH₂— | 2-NH₂ |
| 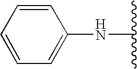 | 5-F | H | H | —CH₂— | 2-NH₂ |
| 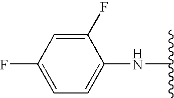 | 5-F | H | H | —CH₂— | 2-NH₂ |
| 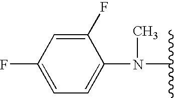 | 5-F | H | H | —CH₂— | 2-NH₂ |
| 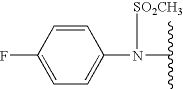 | 5-F | H | H | —CH₂— | 2-NH₂ |
| 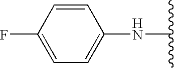 | 5-F | H | H | —CH₂— | 2-NH₂ |
| 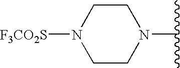 | 5-F | H | H | —CH₂— | 2-NH₂ |
| CH₃—O—(CH₂)₂—NH— | 5-F | H | H | —CH₂— | 2-NH₂ |
| 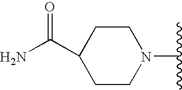 | 5-F | H | H | —CH₂— | 2-NH₂ |
| 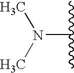 | 5-F | H | H | —CH₂— | 2-NH₂ |
| CH₃—O— | 5-F | H | H | —CH₂— | 2-NH₂ |
| CH₃—CH₂—O— | 5-F | H | H | —CH₂— | 2-NH₂ |
| CH₃—O—(CH₂)₂—O— | 5-F | H | H | —CH₂— | 2-NH₂ |
| (CH₃)₂—CH—O— | 5-F | H | H | —CH₂— | 2-NH₂ |
| 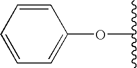 | 5-F | H | H | —CH₂— | 2-NH₂ |
| 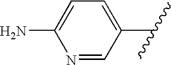 | H | H | H | —CH₂— | 2-NH₂ |
| 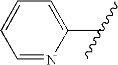 | 5-CF₃,7-F | H | H | —CH₂— | 2-NH₂ |
| 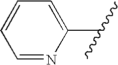 | 5-F | H | H | 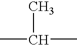 | 2-NH₂ |
| 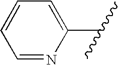 | 5-F | F | H | —CH₂— | 2-NH₂ |
| 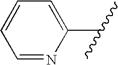 | 5-F | OH | H | —CH₂— | 2-NH₂ |
| 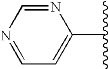 | 5-F | H | H | 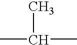 | 2-NH₂ |
| 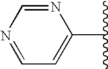 | 5-F | H | H | 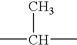 | 2-NH₂ |
| 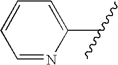 | 5-F | —CH₃ | H | —CH₂— | 2-NH₂ |
| 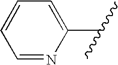 | 6-F | H | H | 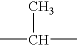 | 2-NH₂ |
| H | 5-F | H | H | —CH₂— | 2-NH₂ |
| 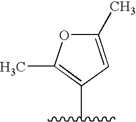 | 5-F | H | H | —CH₂— | 2-NH₂ |
| 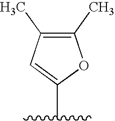 | 5-F | H | H | —CH₂— | 2-NH₂ |
| 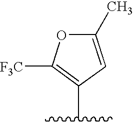 | 5-F | H | H | —CH₂— | 2-NH₂ |
| 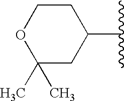 | 5-F | H | H | —CH₂— | 2-NH₂ |
| 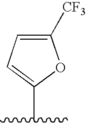 | 5-F | H | H | —CH₂— | 2-NH₂ |
| 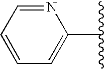 | H | F | H | —CH₂— | 2-NH₂ |
| (CH₃)₂N—(CH₂)₂—NH— | 5-F | H | H | —CH₂— | 2-NH₂ |
| CH₃—S— | 5-F | H | H | 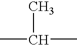 | 2-NH₂ |
| 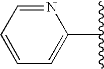 | 5-F | H | 2-CH₃ | —CH₂— | 2-NH₂ |
| 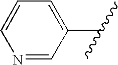 | 5-F | H | H | —CH₂— | 2-NH₂ |
| 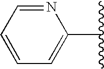 | 5-F | H | H | —CH₂— | 3-NH₂ |
| 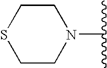 | 5-F | H | H | —CH₂— | 2-NH₂ |
| 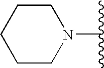 | 5-F | H | H | —CH₂— | 2-NH₂ |
| CH₃CH₂O— | 5-F | F | H | —CH₂— | 2-NH₂ |
| 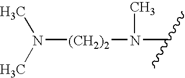 | 5-F | H | H | —CH₂— | 2-NH₂ |
| 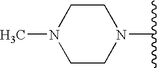 | 5-F | H | H | —CH₂— | 2-NH₂ |
| 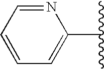 | 5-F | H | 5-OH | —CH₂— | 2-NH₂ |
| 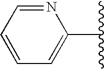 | 5-F | F | H | —CH₂— | 3-NH₂ |
| 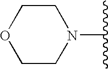 | 5-F | F | H | —CH₂— | 2-NH₂ |
| 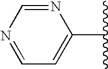 | 5-F | H | H | —CH₂— | 3-NH₂ |

compounds having the following general formula:

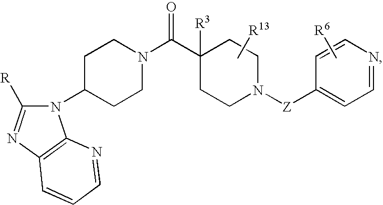

wherein R, R³, Z, and R⁶ are as defined in the following Table:

| R | R³ | Z | R⁶ |
|---|---|---|---|
| 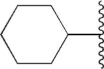 | H | —CH₂— | 2-NH₂ |
| —CH₂OCH₃ | H | —CH₂— | 2-NH₂ |
| 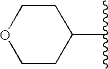 | H | —CH₂— | 2-NH₂ |
| 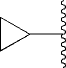 | H | —CH₂— | 2-NH₂ |
| (CH₃)₂—CH— | H | —CH₂— | 2-NH₂ |
| 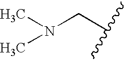 | H | —CH₂— | 2-NH₂ |
| 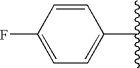 | H | —CH₂— | 2-NH₂ |
| 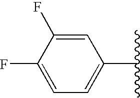 | H | —CH₂— | 2-NH₂ |
| 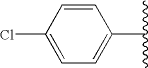 | H | —CH₂— | 2-NH₂ |
| 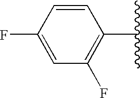 | H | —CH₂— | 2-NH₂ |
| 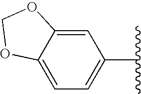 | H | —CH₂— | 2-NH₂ |
| 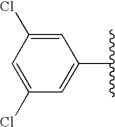 | H | —CH₂— | 2-NH₂ |
| 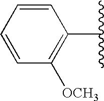 | H | —CH₂— | 2-NH₂ |
| 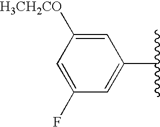 | H | —CH₂— | 2-NH₂ |
| 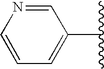 | H | —CH₂— | 2-NH₂ |
| 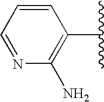 | H | —CH₂— | 2-NH₂ |
| 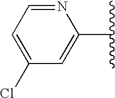 | H | —CH₂— | 2-NH₂ |
| 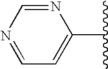 | H | —CH₂— | 2-NH₂ |
| 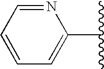 | H | —CH₂— | 2-NH₂ |
| 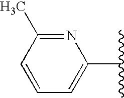 | H | —CH₂— | 2-NH₂ |
| 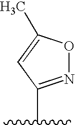 | H | —CH₂— | 3-NH₂ |
| 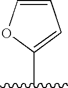 | H | —CH₂— | 2-NH₂ |
| 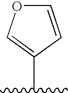 | H | —CH₂— | 2-NH₂ |
| 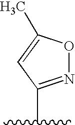 | H | —CH₂— | 2-NH₂ |
| 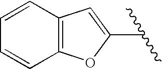 | H | —CH₂— | 2-NH₂ |
| 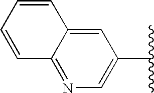 | H | —CH₂— | 2-NH₂ |
| 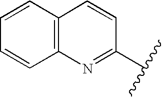 | H | —CH₂— | 2-NH₂ |
| 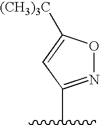 | H | —CH₂— | 2-NH₂ |
| 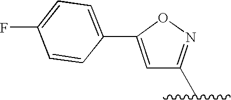 | H | —CH₂— | 2-NH₂ |
| 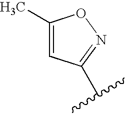 | F | —CH₂— | 2-NH₂ |
| 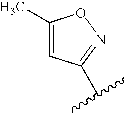 | H | 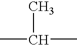 | 2-NH₂ |
| 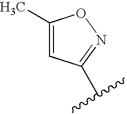 | OH | —CH₂— | 2-NH₂ |
| 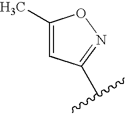 | 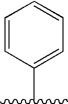 | —CH₂— | 2-NH₂ |
| 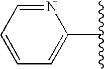 | F | —CH₂— | 2-NH₂ |
| 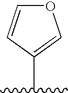 | F | —CH₂— | 2-NH₂ |
| 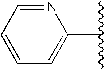 | H | —CH₂— | 3-NH₂ |
| 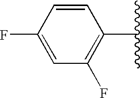 | H | —CH₂— | 3-NH₂ |
| 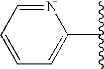 | F | —CH₂— | 3-NH₂ |
| 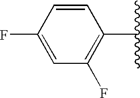 | F | —CH₂— | 3-NH₂ |

compounds of the following general formula:

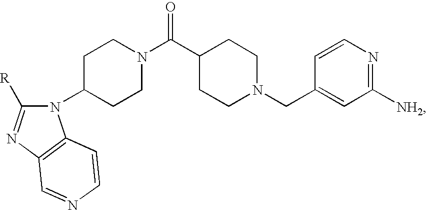

wherein R is as defined in the following Table:

R
—CH₃

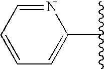

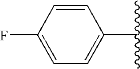

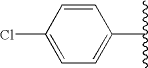

compounds of the following general formula:

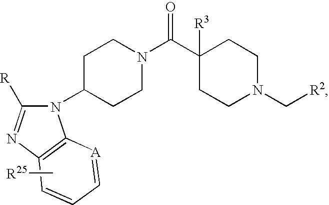

wherein R, R²⁵, A, R³, and R² are as defined in the following Table:

| R | R²⁵ | A | R³ | R² |
|---|---|---|---|---|
| 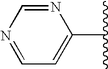 | 5-Cl | C | H | 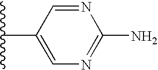 |
| 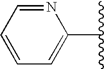 | 5-F | C | H | 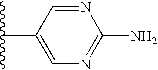 |
| 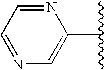 | 5-Cl | C | H | 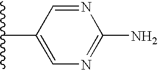 |
| 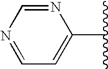 | 5-F | C | H | 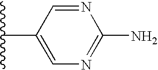 |
| 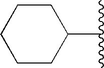 | H | N | H | 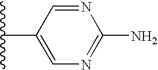 |
| 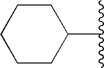 | H | N | H | 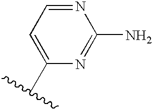 |
| (CH₃)₂CH— | H | N | H | 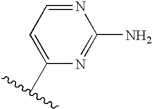 |
| 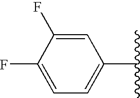 | 5-F | C | H | 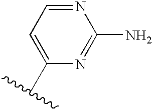 |
| 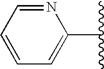 | 5-F | C | H | 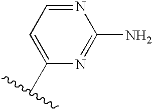 |
| 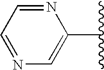 | 5-Cl | C | H | 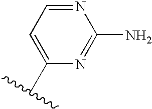 |
| 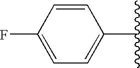 | 6-Cl | C | H | 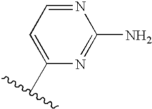 |
| 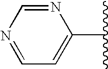 | 5-F | C | H | 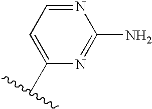 |
| 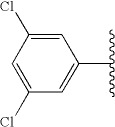 | 6-Cl | C | H | 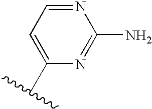 |
| 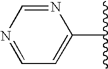 | 5-Cl | C | H | 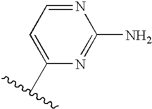 |
| 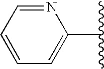 | 6-F | C | H | 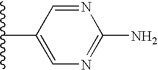 |
| 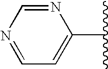 | H | N | H | 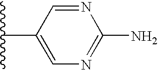 |
| 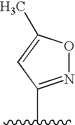 | H | N | H | 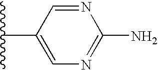 |
| 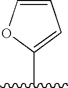 | H | N | H | 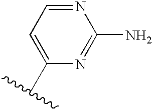 |
| 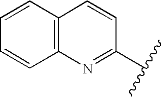 | H | N | H | 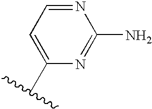 |
| 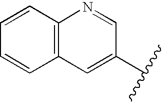 | H | N | H | 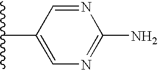 |
| 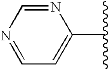 | H | N | H | 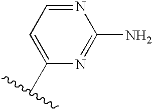 |
| 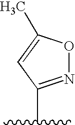 | H | N | H | 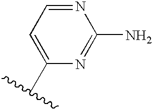 |
| 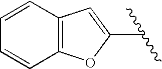 | H | N | H | 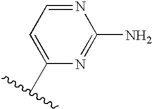 |
| 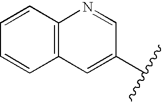 | H | N | H | 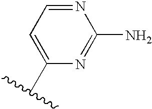 |
| 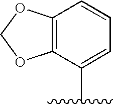 | H | N | H | 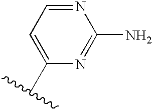 |
| 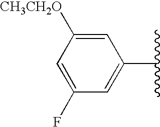 | H | N | H | 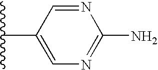 |
| 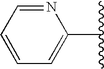 | H | N | H | 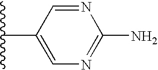 |
| 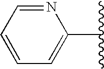 | 5-F | C | F | 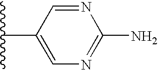 |
| 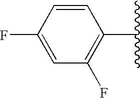 | 5-F | C | H | 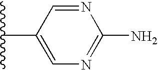 |
| 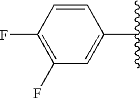 | 5-F | C | H | 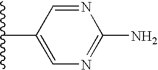 |
| 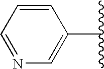 | 5-F | C | H | 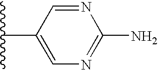 |
| 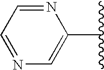 | 5-F | C | H | 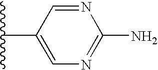 |
| 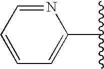 | H | C | H | 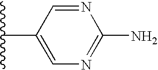 |
| (CH₃)₂N—CH₂— | H | N | H | 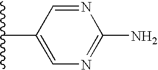 |
| 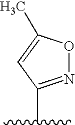 | 5-F | C | H | 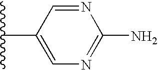 |
| 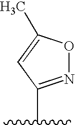 | H | C | H | 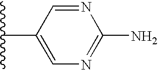 |
| 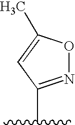 | 5,6-di-F | C | H | 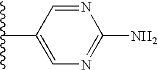 |
| 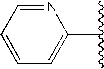 | 5-F | C | H | 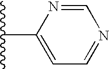 |
| 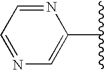 | 5,6-di-F | C | H | 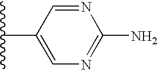 |
| 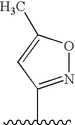 | 5-F | C | F | 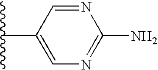 |
| 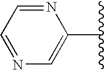 | 5-F | C | F | 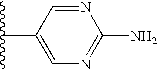 |
| 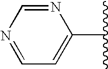 | 5-F | C | F | 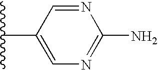 |
| 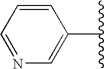 | 5-F | C | F | 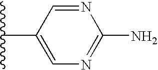 |
| 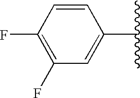 | 5-F | C | F | 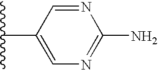 |
| 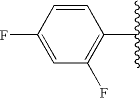 | 5-F | C | F | 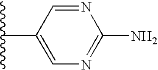 |
| 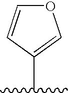 | H | N | H | 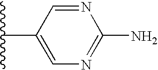 |
| 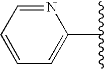 | H | C | F | 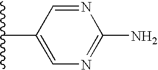 |
| 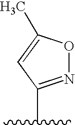 | H | C | F | 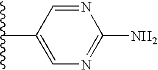 |
| 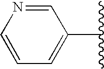 | H | N | F | 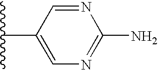 |
| 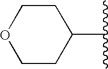 | H | N | H | 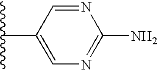 |
| 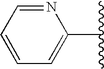 | H | N | F | 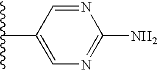 |
| 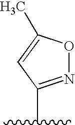 | H | N | F | 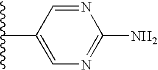 |
| 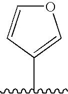 | 5-F | C | H | 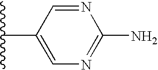 |
| 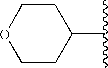 | 5-F | C | H | 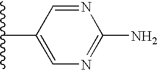 |
| 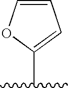 | 5-F | C | H | 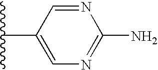 |
| 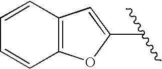 | H | N | H | 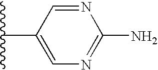 |
| (CH₃)₂N—CH₂— | H | N | F | 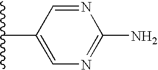 |
| 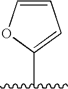 | H | N | F | 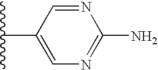 |
| CH₃CH₂—O— | 5-F | C | H | 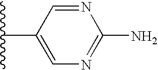 |
| CH₃—S— | 5-F | C | H | 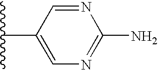 |
| CH₃CH₂—O— | 5-F | C | F | 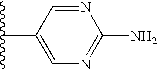 |
| 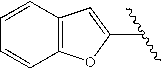 | H | N | F | 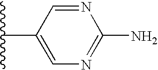 |
| 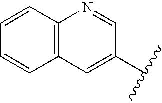 | H | N | F | 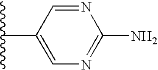 |
| 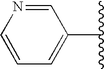 | H | N | H | 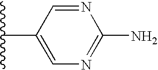 |
| 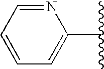 | 5,6-di-F | C | F | 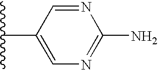 |
| 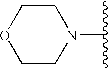 | 5-F | C | F | 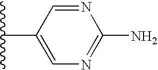 |
| 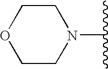 | 5-F | C | H | 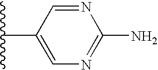 |
| 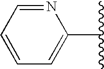 | 5-F | C | H | 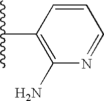 |
| 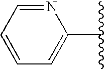 | 5-F | C | H | 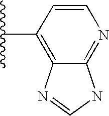 |
| 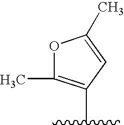 | H | N | H | 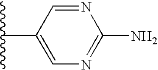 |
| 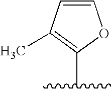 | H | N | H | 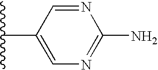 |
| 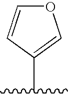 | H | N | F | 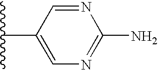 |
| 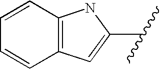 | H | N | H | 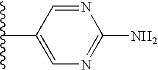 |
| 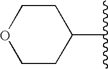 | H | N | F | 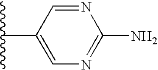 |
| 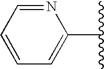 | 5-F | C | F | 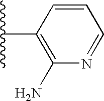 |
| 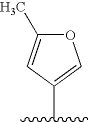 | H | N | H | 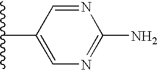 |
| 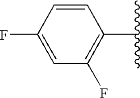 | H | N | H | 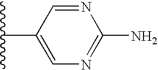 |
| 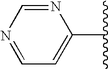 | H | N | F | 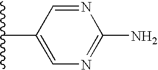 |
| 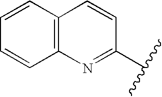 | H | N | H | 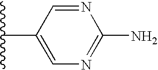 |
| 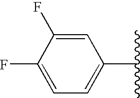 | H | N | H | 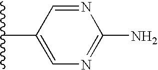 |
| CH₃S— | 5-F | C | F | 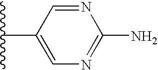 |
| 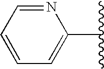 | H | N | F | 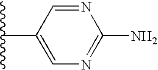 |
| 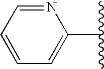 | 5-F | C | F | 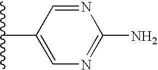 |
| 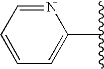 | 5-F | C | H | 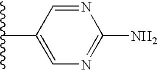 |
| 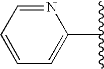 | H | N | H | 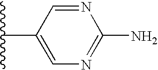 |
| (CH₃)₂N— | 5-F | C | F | 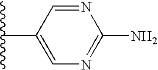 |
| CH₃CH₂—S— | 5-F | C | F | 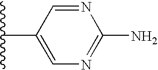 |
| CH₃—O— | 5-F | C | F | 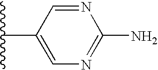 |
| 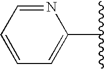 | H | N | H | 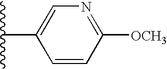 |
| 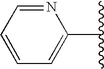 | H | N | F | 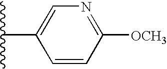 |
| 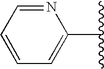 | 5-F | C | F | 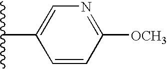 |
| 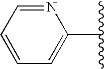 | 5-F | C | H | 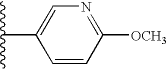 |
| 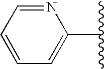 | 5-F | C | H | 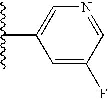 |
| 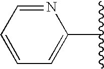 | 5-F | C | F | 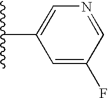 |
| 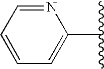 | H | N | H | 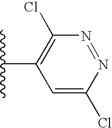 |
| 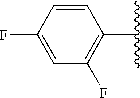 | H | N | F | 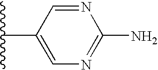 |
| 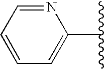 | 5-F | C | H | 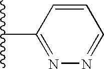 |
| 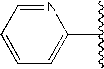 | 5-F | C | H | 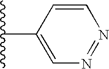 |
| 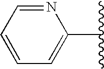 | 5-F | C | F | 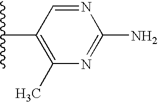 |
| (CH₃CH₂)₂N— | 5-F | C | F | 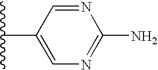 |
| 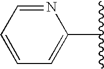 | H | N | H | 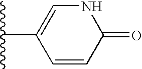 |
| 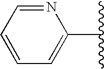 | H | N | F | 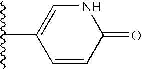 |
| 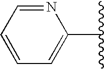 | 5-F | C | H | 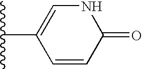 |
| 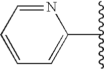 | 5-F | C | F | 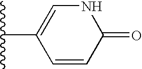 |
| 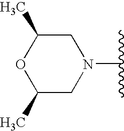 | 5-F | C | F | 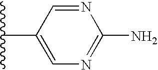 |
| CH₃—S— | H | N | F | 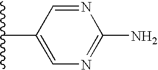 |
| CH₃CH₂—O— | H | N | F | 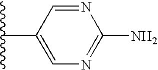 |
| 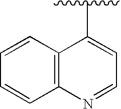 | H | N | F | 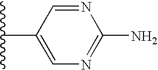 |
| 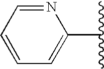 | H | N | F | 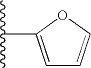 |
| 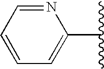 | H | N | F | 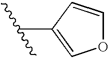 |
| 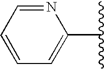 | H | N | F | 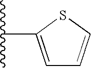 |
| 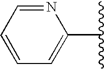 | H | N | F | 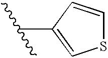 |
| 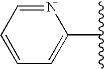 | 5-F | C | F | 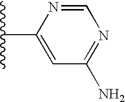 |
| 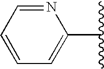 | H | N | F | 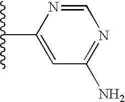 |
| 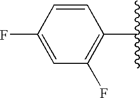 | H | N | F | 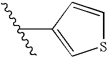 |
| 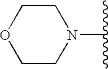 | H | N | F | 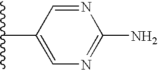 |
| (CH₃)₂CH—O— | 5-F | C | F | 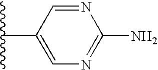 |
| 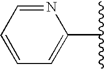 | H | N | F | 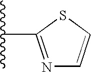 |
| 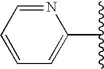 | H | N | F | 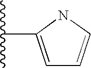 |
| 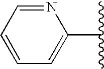 | H | N | F | 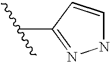 |
| 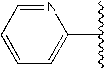 | H | N | F | 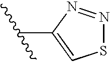 |
| 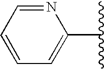 | H | N | F | 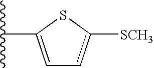 |
| 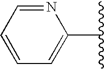 | H | N | F | 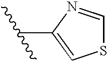 |
| 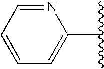 | H | N | F | 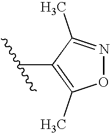 |
| 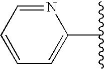 | H | N | F | 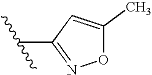 |
| CH₃—O— | H | N | F | 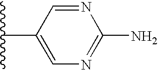 |
| 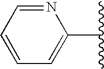 | H | N | F | 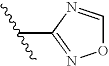 |
| 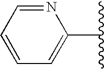 | H | N | F | 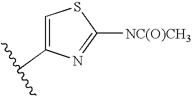 |
| 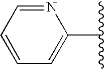 | 5-F | C | H | 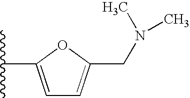 |
| 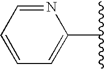 | 5-F | C | F | 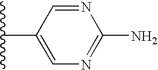 |
| 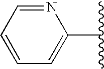 | H | N | F | 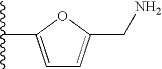 |
| 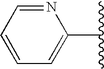 | 5-F | C | H | 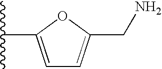 |
| 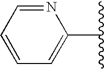 | H | N | F | 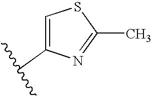 |
| 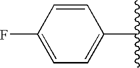 | 6-Cl | C | H | 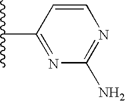 |
| 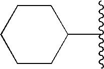 | H | N | H | 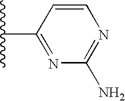 |
| (CH₃)₂—CH— | H | N | H | 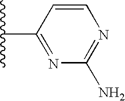 |

compounds of the following general formula:

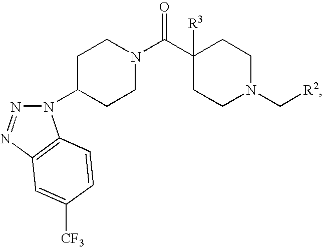

wherein R³ and R² are as defined in the following Table:

| R³ | R² |
|---|---|
| H | 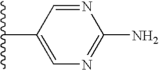 |
| F | 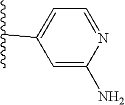 |
| F | 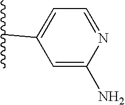 |
| F | 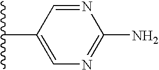 |
| F | 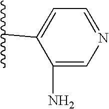 |

compounds of the following general formula:

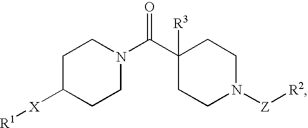

wherein R¹—X—, Z, R³, and R² are defined as shown in the following Table:

| R¹—X— | Z | R³ | R² |
|---|---|---|---|
| 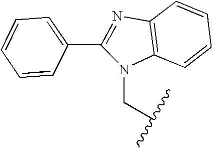 | —CH₂— | H | 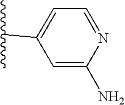 |
| 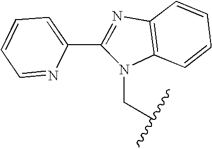 | —CH₂— | H | 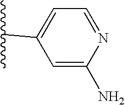 |
| 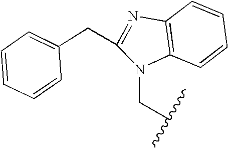 | —CH₂— | H | 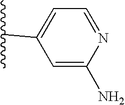 |
| 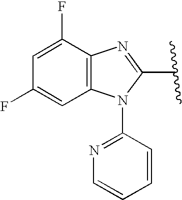 | —CH₂— | H | 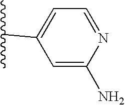 |
| 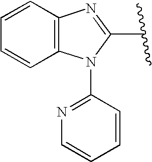 | —CH₂— | H | 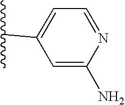 |
| 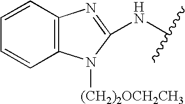 | —CH₂— | H | 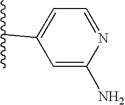 |
| 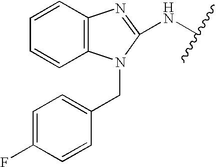 | —CH₂— | H | 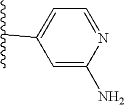 |
| 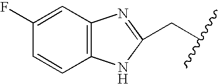 | —CH₂— | H | 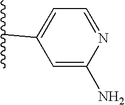 |
| 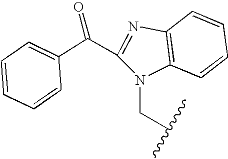 | —CH₂— | H | 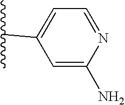 |
| 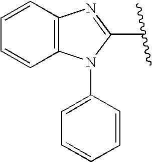 | —CH₂— | H | 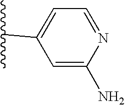 |
| 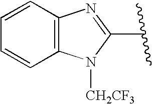 | —CH₂— | H | 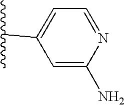 |
| 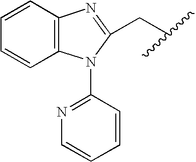 | —CH₂— | H | 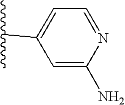 |
| 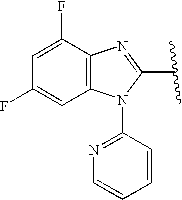 | —CH₂— | H | 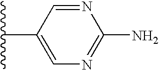 |
| 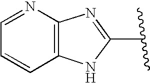 | —CH₂— | H | 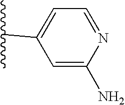 |
| 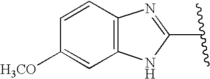 | —CH₂— | H | 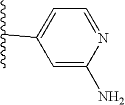 |
| 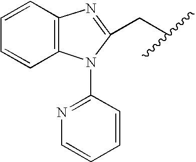 | —CH₂— | H | 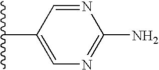 |
| 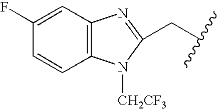 | —CH₂— | H | 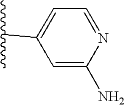 |
| 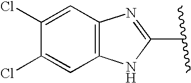 | —CH₂— | H | 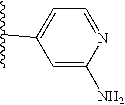 |
| 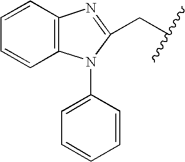 | —CH₂— | H | 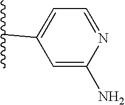 |
| 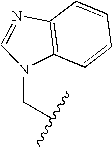 | —CH₂— | H | 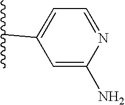 |
| 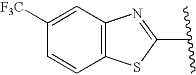 | —CH₂— | H | 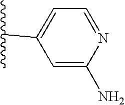 |
| 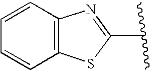 | —CH₂— | H | 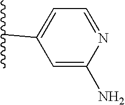 |
| 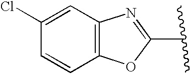 | —CH₂— | H | 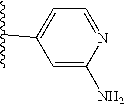 |
| 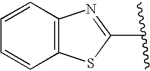 | —(CH₂)₃— | H | 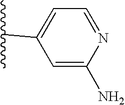 |
| 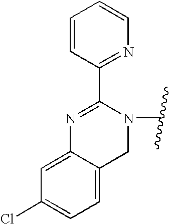 | —CH₂— | H | 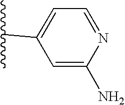 |
| 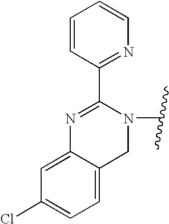 | —CH₂— | F | 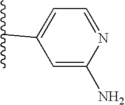 |

compounds of the following general formula:

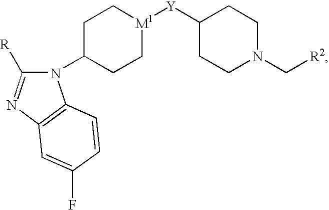

wherein R, M¹, Y, and R² are defined as shown in the following Table:

| R | M¹ | Y | R² |
|---|---|---|---|
| 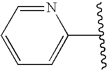 | CH | —CH₂— | 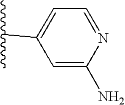 |
| 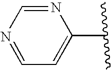 | N | —NH— | 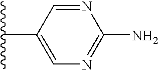 |
| 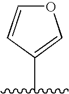 | N | —NH— | 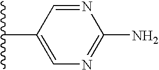 |
| 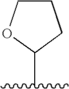 | N | —NH— | 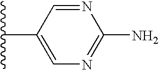 |
| 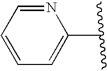 | N | —NH— | 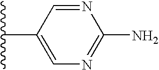 |
| 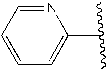 | N | —NH— | 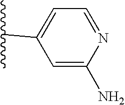 |
| 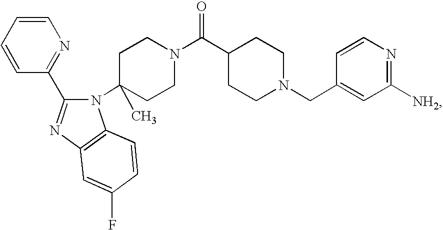 | | | |
| 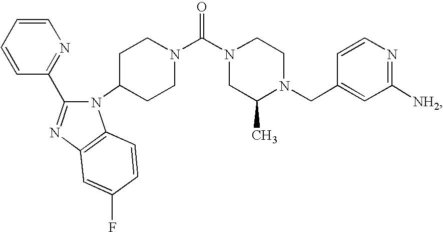 | | | |
| 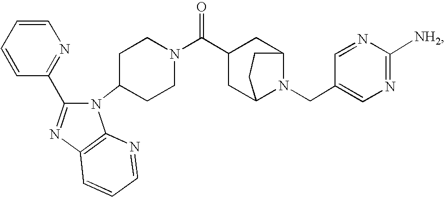 | | | |
| 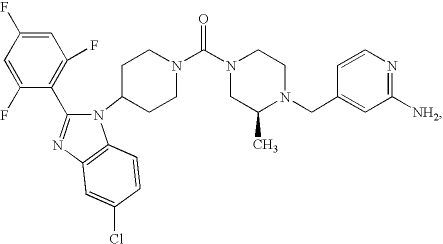 | | | |
| 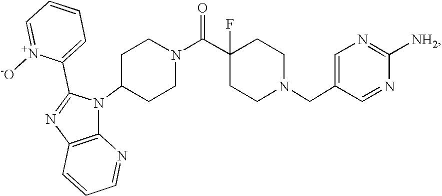 | | | |
| 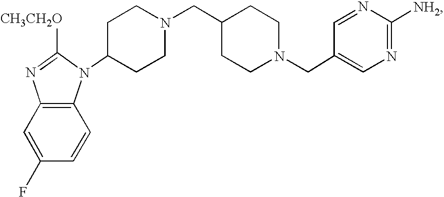 | | | |
| 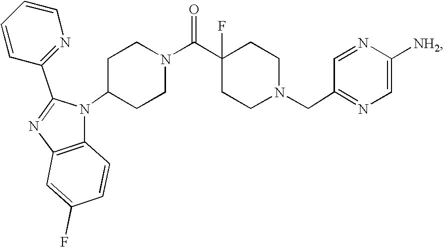 | | | |
| 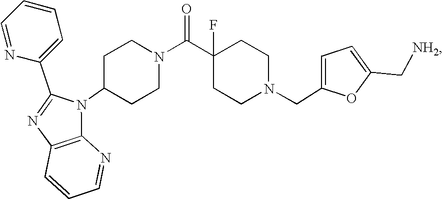 | | | |
| 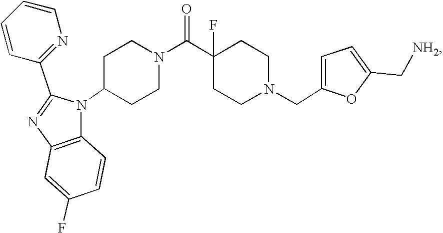 | | | |
| 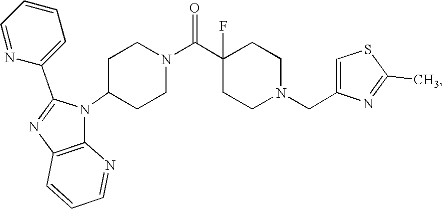 | | | |
| 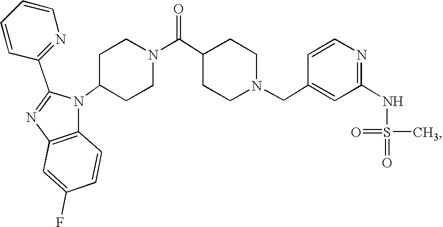 | | | |
| 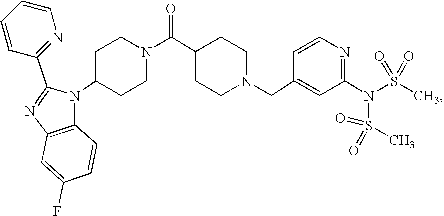 | | | |
| 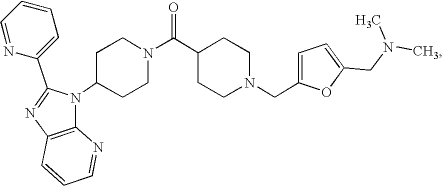 | | | |
| 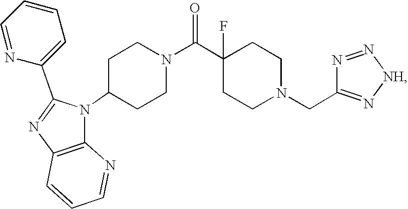 | | | |
| 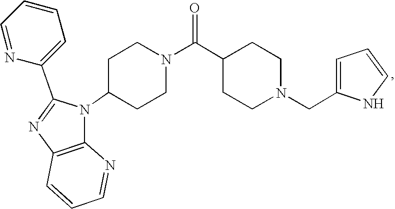 | | | |
| 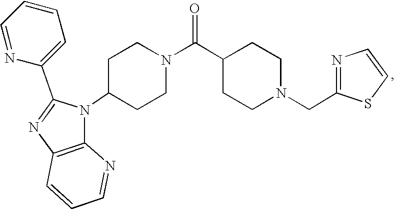 | | | |
| 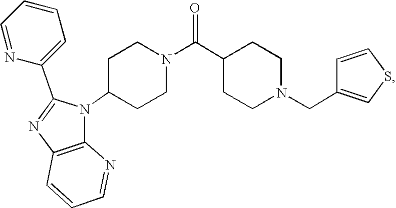 | | | |
| 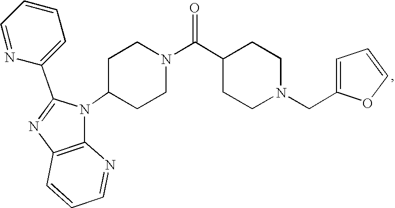 | | | |
| 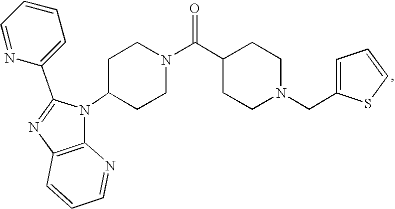 | | | |
| 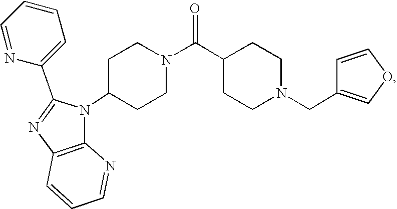 | | | |
| 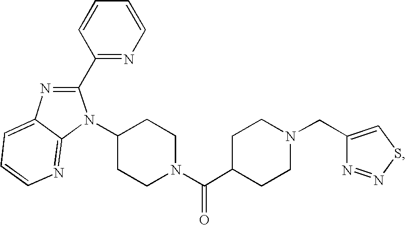 | | | |
| 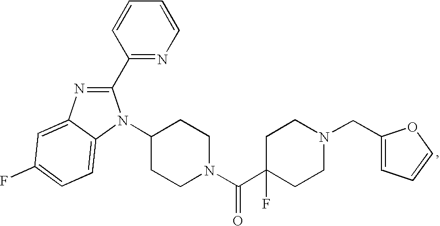 | | | |
| 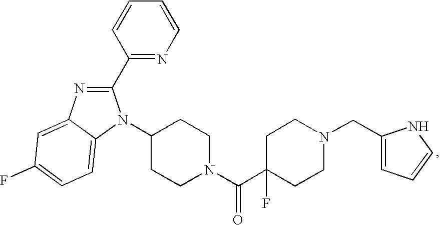 | | | |
| 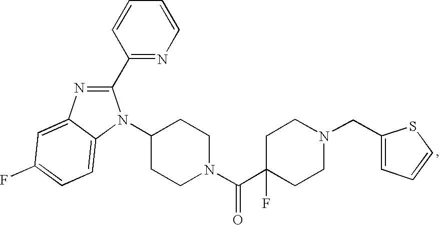 | | | |
| 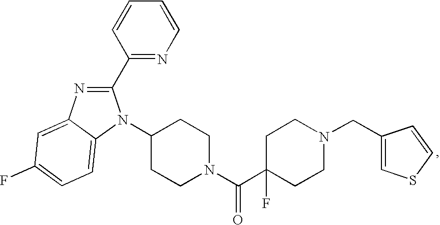 | | | |
| 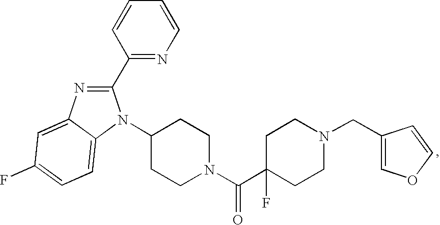 | | | |
| 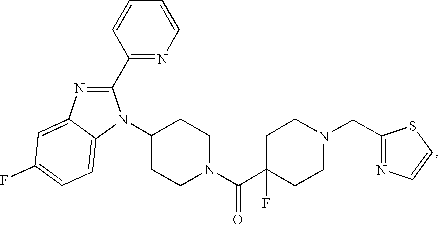 | | | |
| 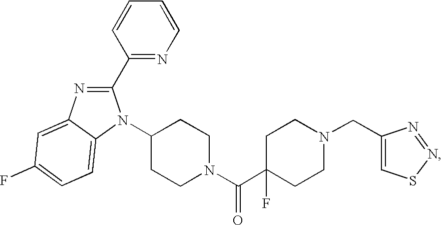 | | | |
| 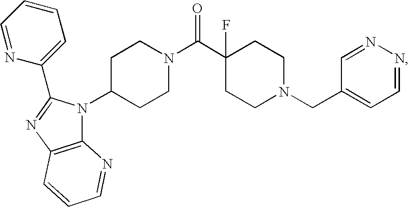 | | | |
| 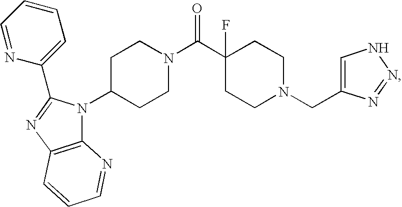 | | | |
| 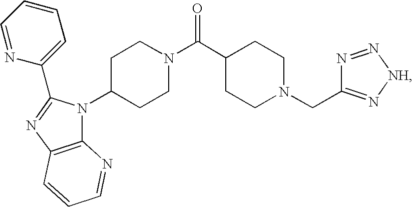 | | | |
| 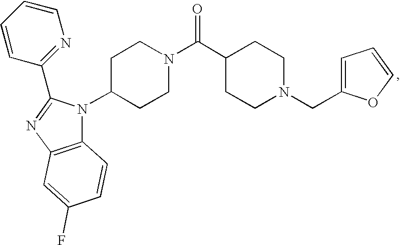 | | | |
| 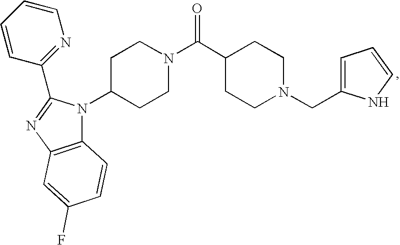 | | | |
| 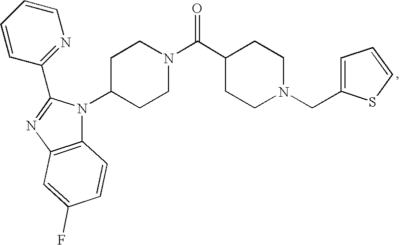 | | | |
| 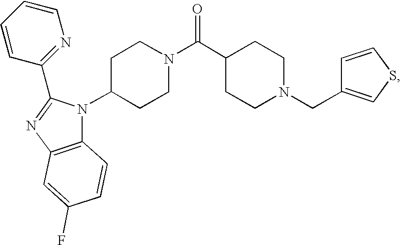 | | | |
| 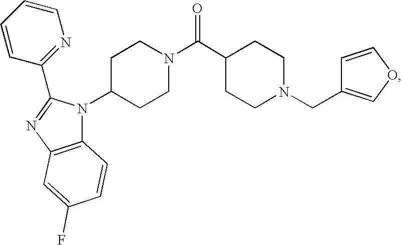 | | | |
| 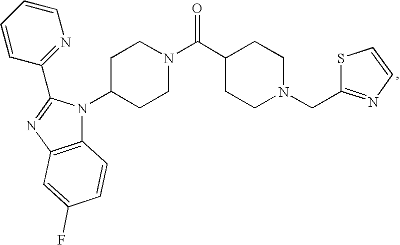 | | | |
| 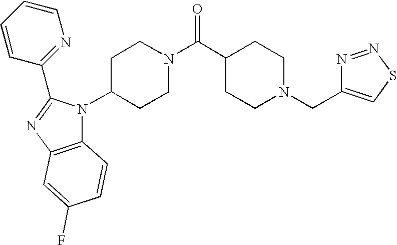 | | | |
| 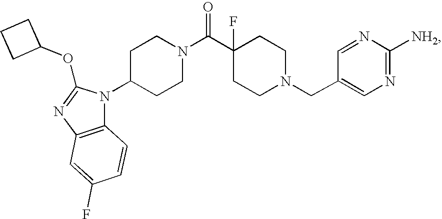 | | | |
| 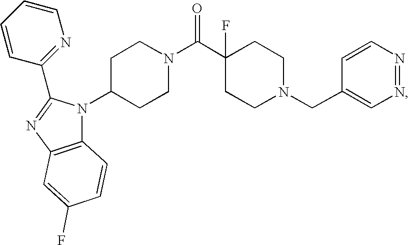 | | | |
| 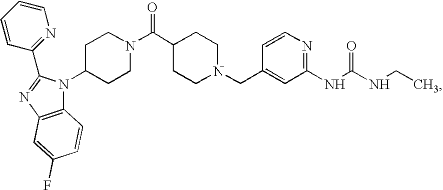 | | | |
| 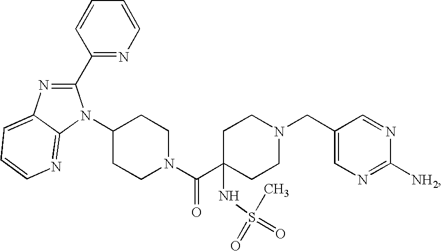 | | | |
| 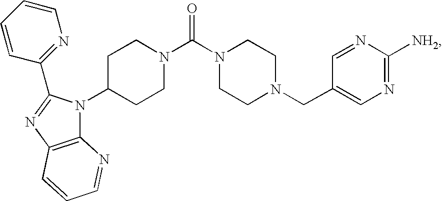 | | | |
| 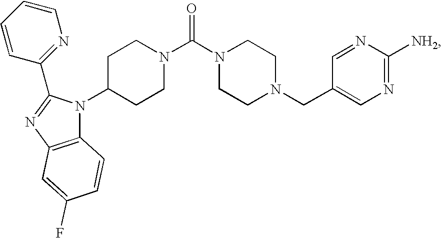 | | | |
| 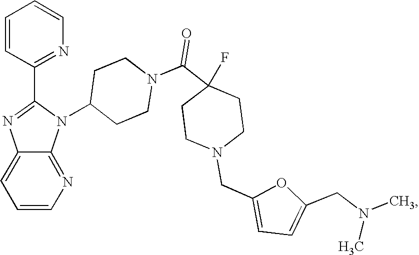 | | | |
| 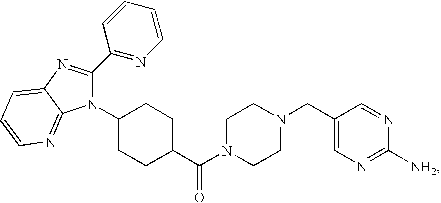 | | | |
| 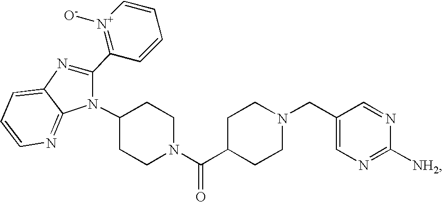 | | | |
| 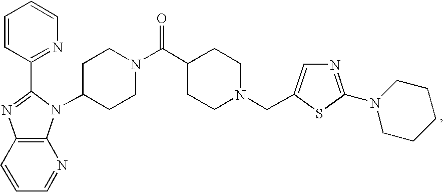 | | | |
| 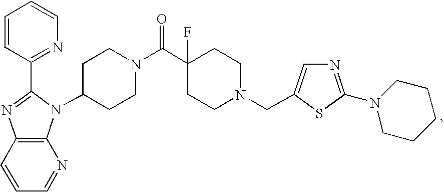 | | | |
| 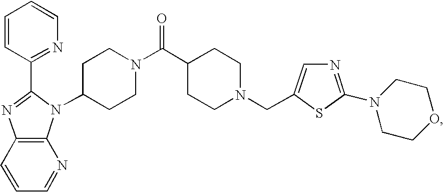 | | | |
| 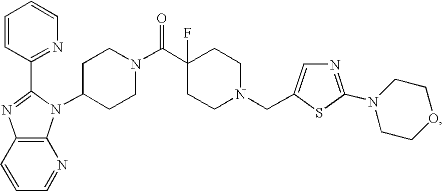 | | | |
| 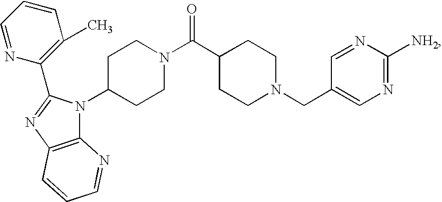 | | | |
| 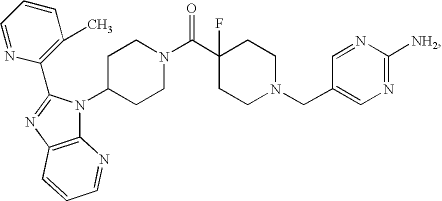 | | | |
| 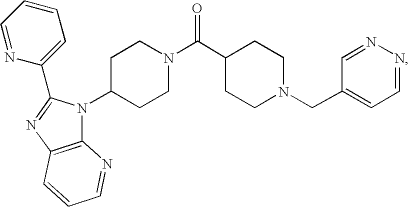 | | | |
| 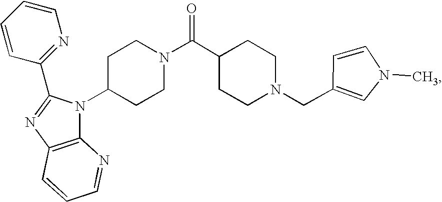 | | | |
| 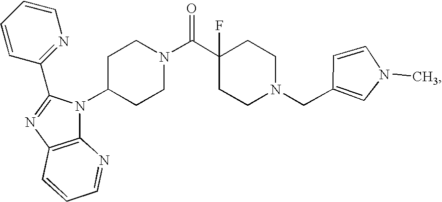 | | | |
| 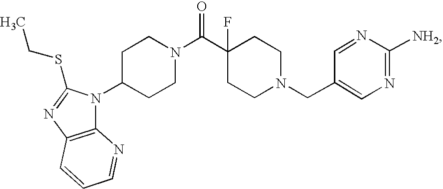 | | | |
| 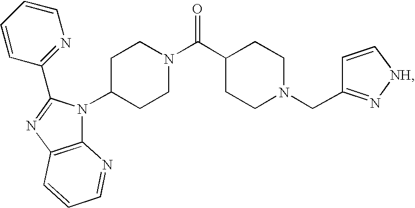 | | | |
| 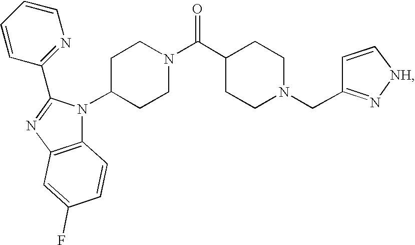 | | | |
| 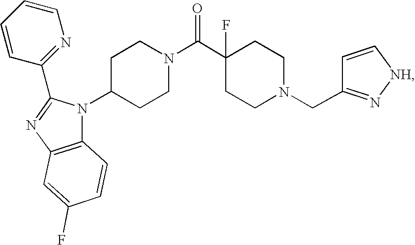 | | | |
| 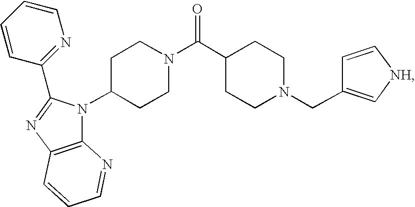 | | | |
| 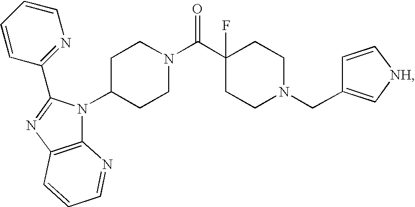 | | | |
| 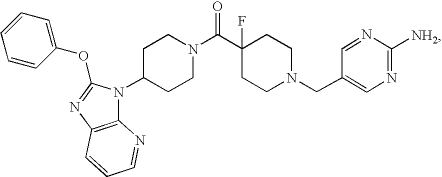 | | | |
| 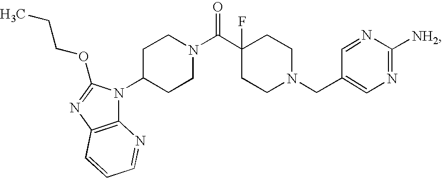 | | | |
| 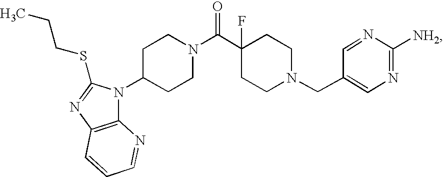 | | | |
| 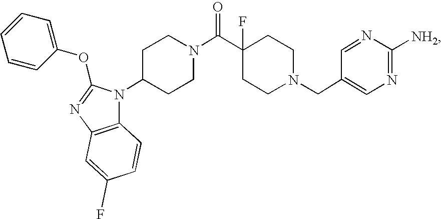 | | | |
| 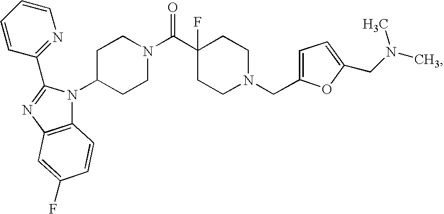 | | | |
| 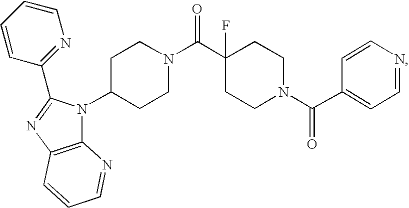 | | | |
| 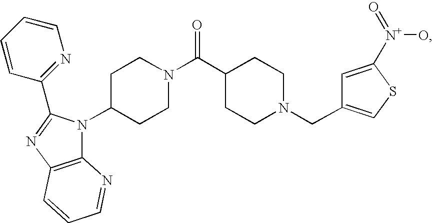 | | | |
| 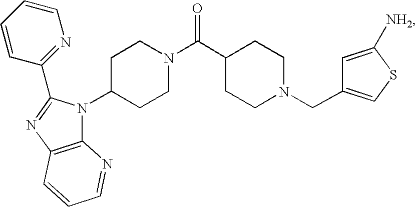 | | | |
| 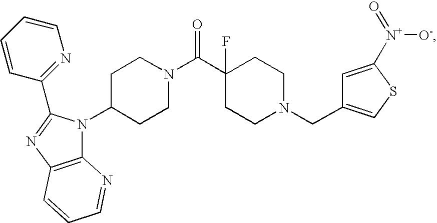 | | | |
| 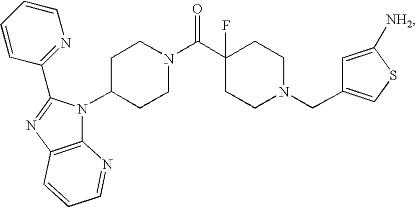 | | | |
| 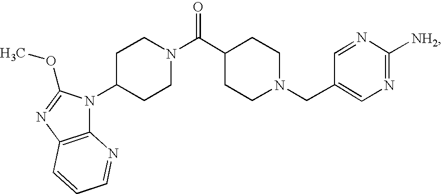 | | | |
| 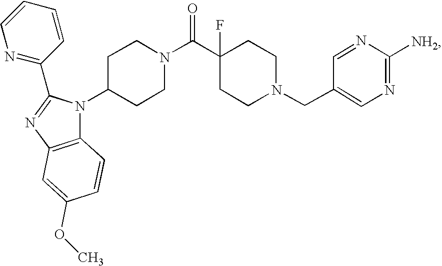 | | | |
| 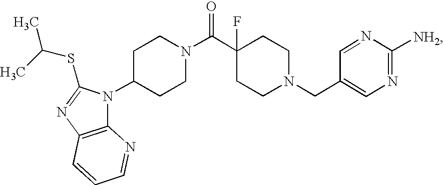 | | | |
| 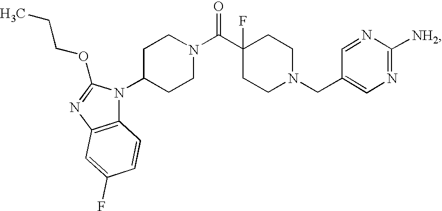 | | | |
| 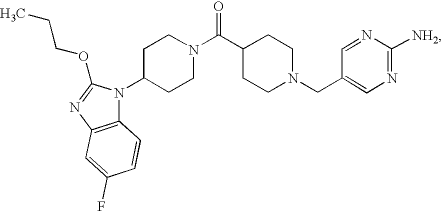 | | | |
| 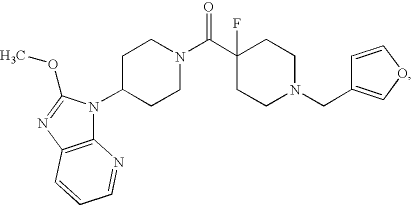 | | | |
| 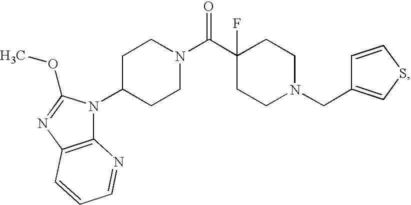 | | | |
| 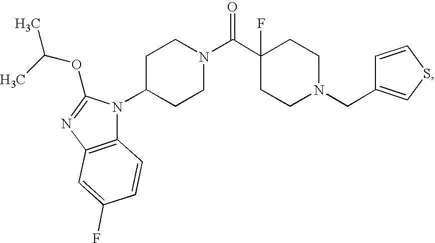 | | | |
| 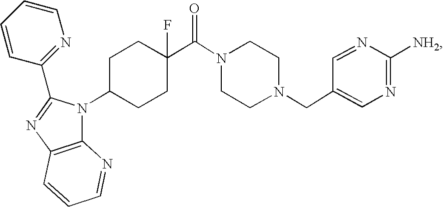 | | | |
| 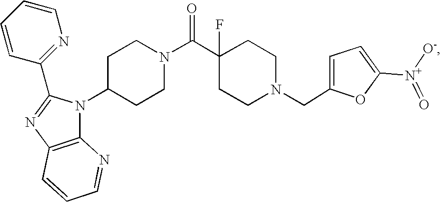 | | | |
| 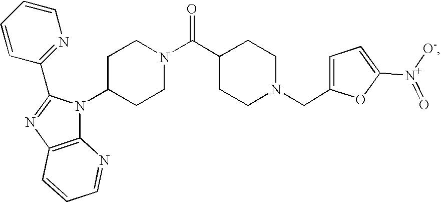 | | | |
| 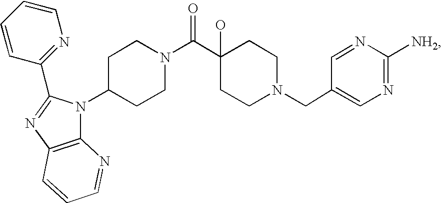 | | | |
| 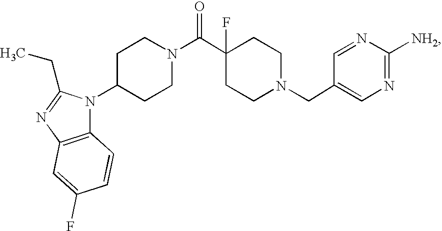 | | | |
| 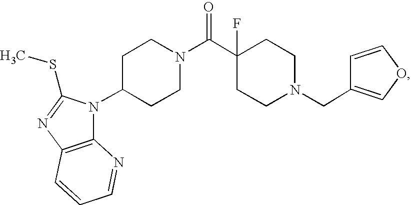 | | | |
| 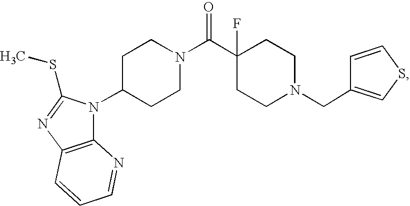 | | | |
| 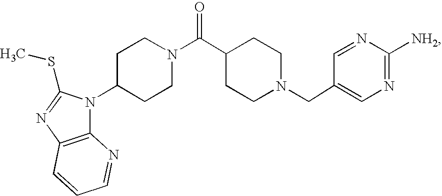 | | | |
| 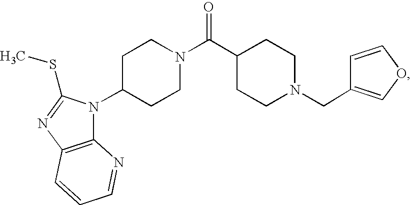 | | | |
| 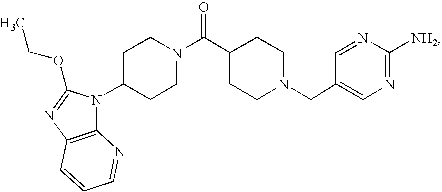 | | | |
| 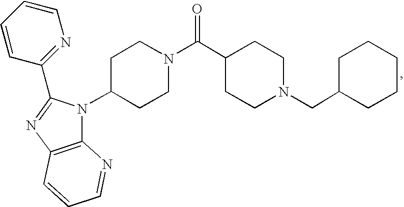 | | | |
| 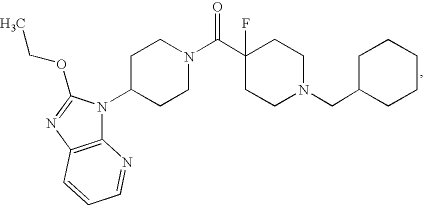 | | | |
| 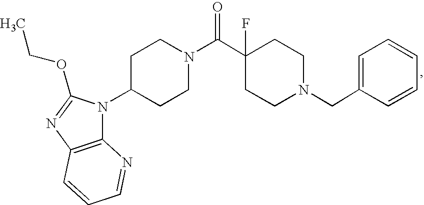 | | | |
| 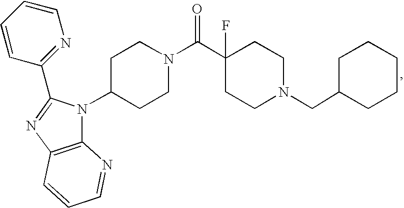 | | | |
| 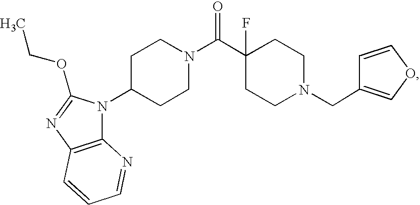 | | | |
| 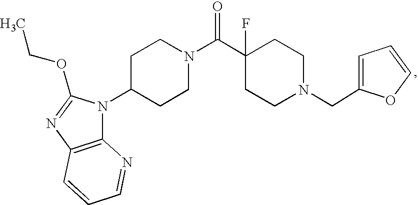 | | | |
| 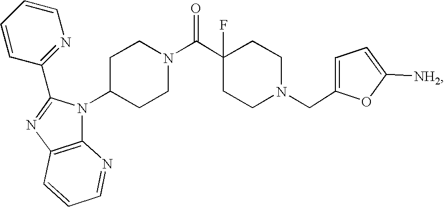 | | | |
| 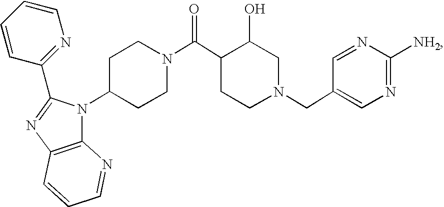 | | | |
| 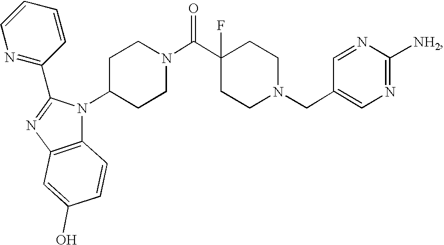 | | | |
| 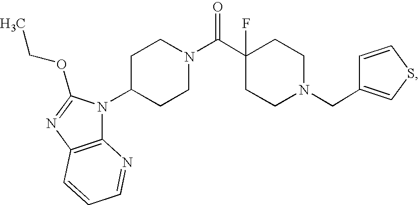 | | | |
| 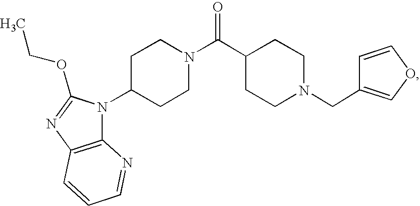 | | | |
| 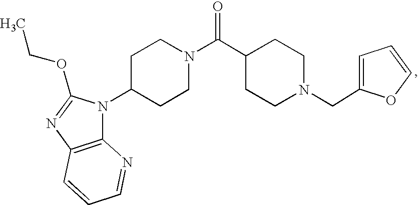 | | | |
| 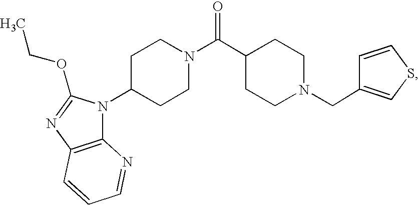 | | | |
| 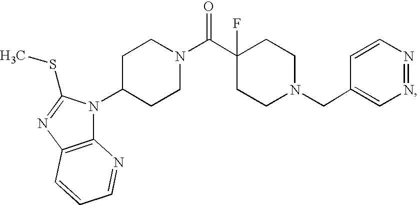 | | | |
| 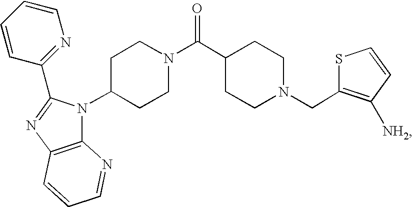 | | | |
| 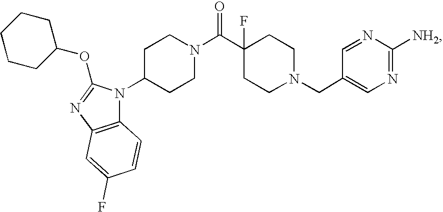 | | | |
| 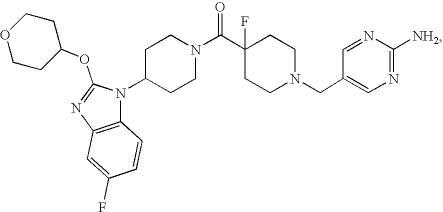 | | | |
| 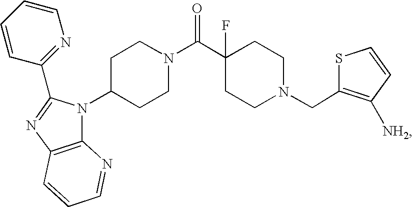 | | | |
| 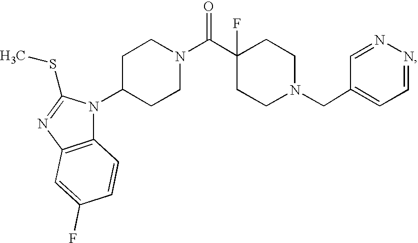 | | | |
| 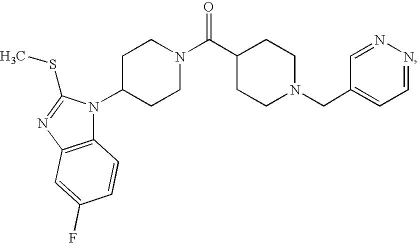 | | | |
| 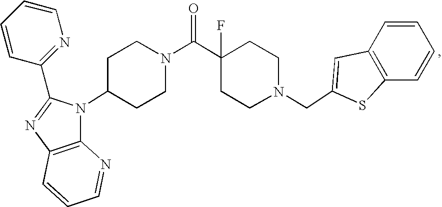 | | | |
| 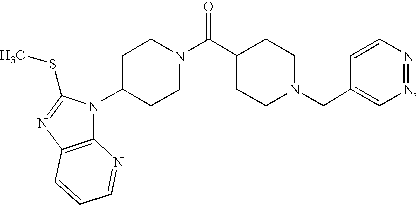 | | | |
| 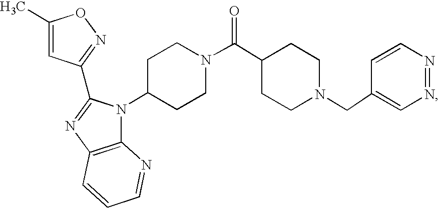 | | | |
| 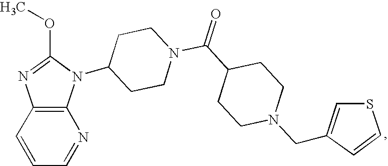 | | | |
| 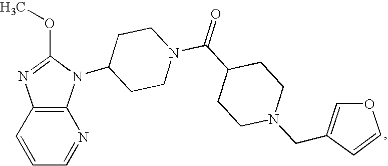 | | | |
| 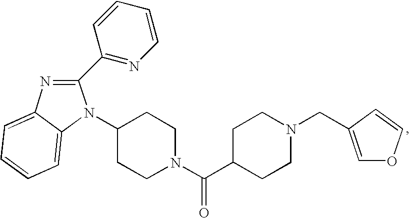 | | | |
| 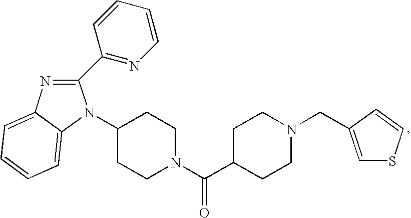 | | | |
| 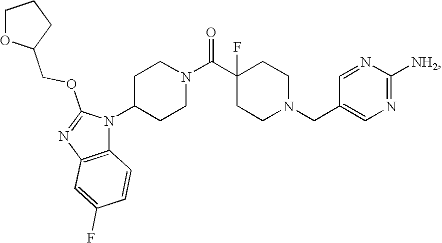 | | | |
| 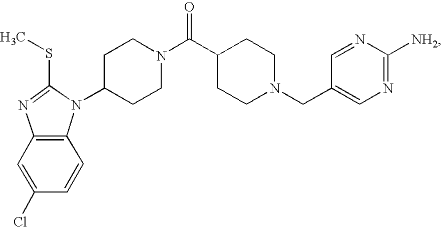 | | | |
| 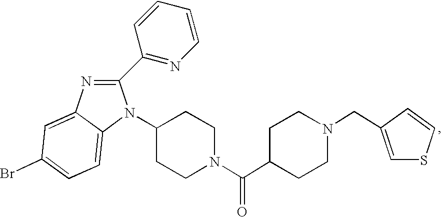 | | | |
| 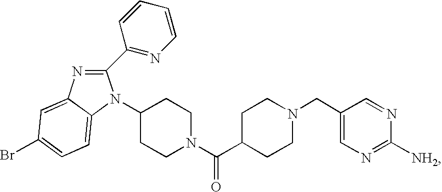 | | | |
| 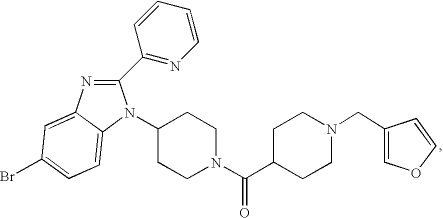 | | | |
| 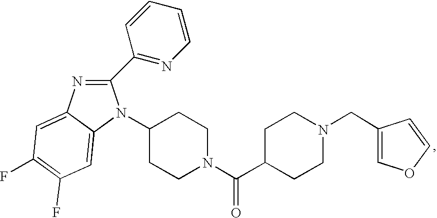 | | | |
| 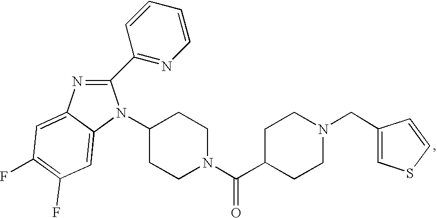 | | | |
| 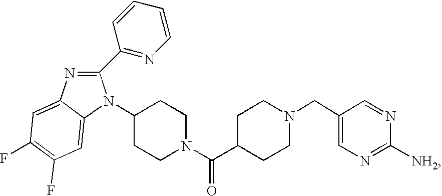 | | | |
| 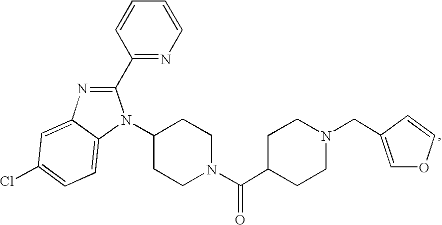 | | | |
| 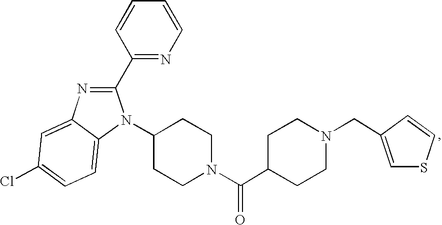 | | | |
| 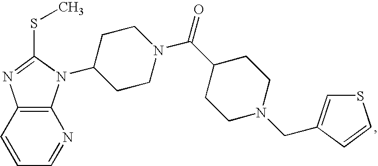 | | | |
| 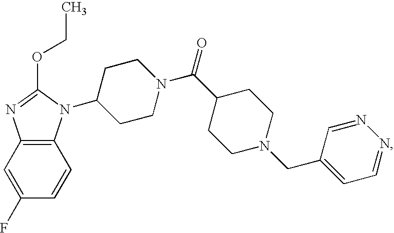 | | | |
| 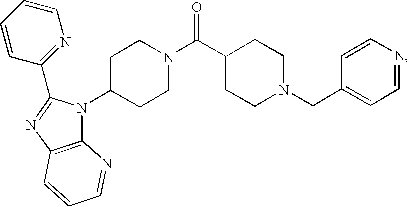 | | | |
| 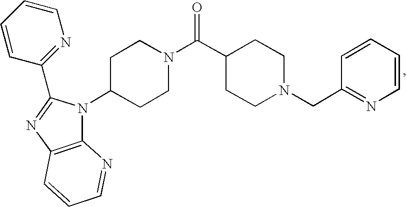 | | | |
| 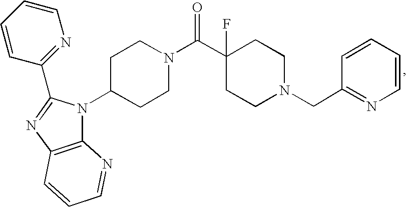 | | | |
| 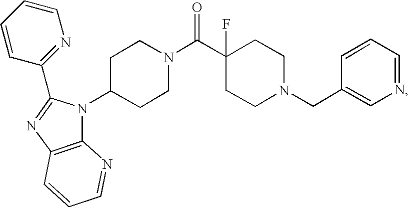 | | | |
| 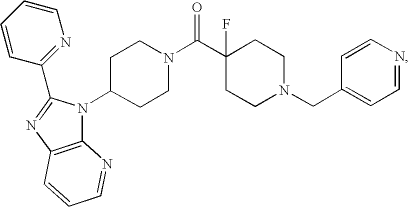 | | | |
| 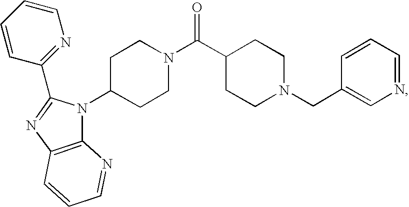 | | | |
| 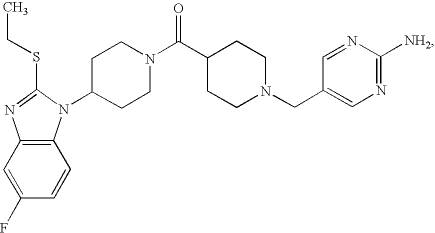 | | | |
| 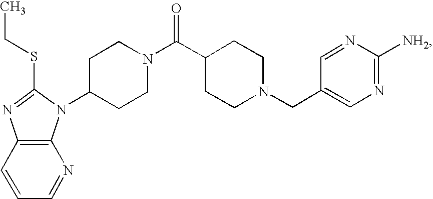 | | | |
| 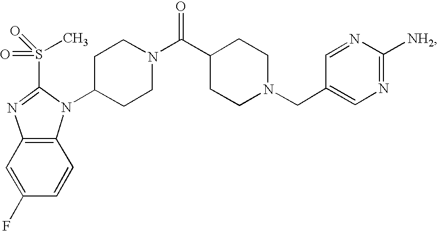 | | | |
| 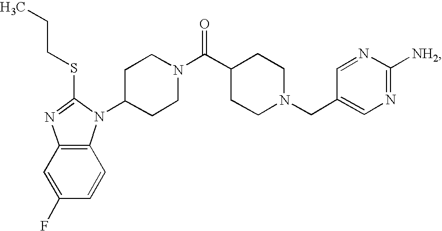 | | | |
| 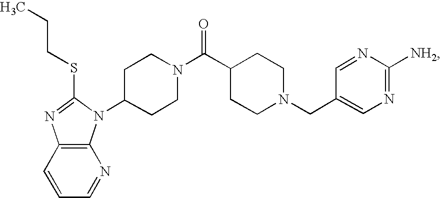 | | | |
| 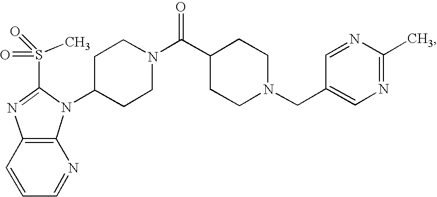 | | | |
| 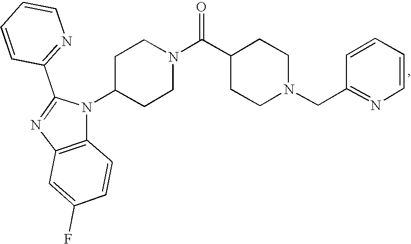 | | | |
| 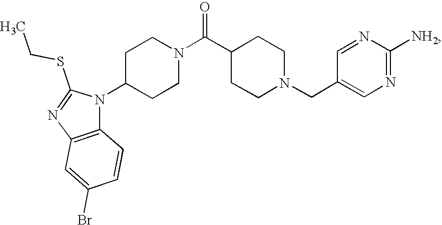 | | | |
| 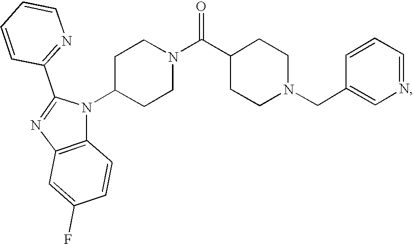 | | | |
| 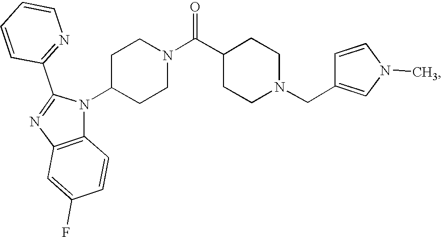 | | | |
| 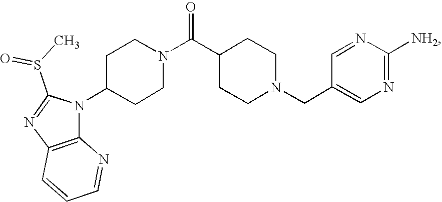 | | | |
| 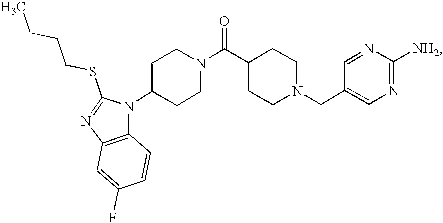 | | | |
| 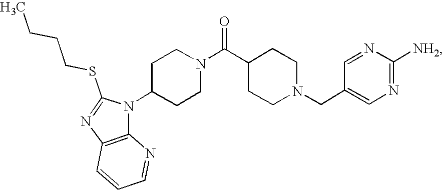 | | | |
| 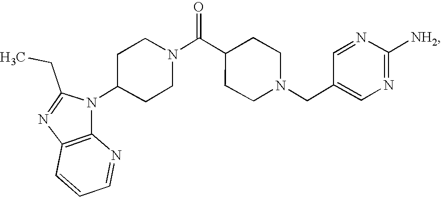 | | | |
| 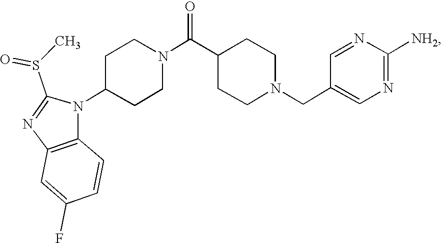 | | | |
| 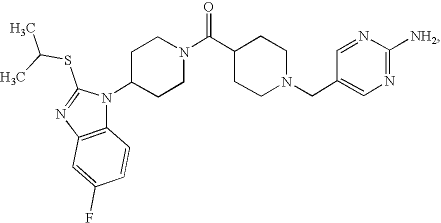 | | | |
| 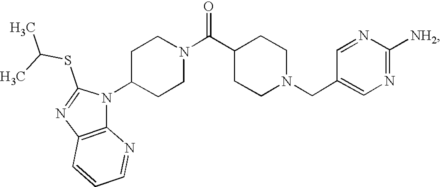 | | | |
| 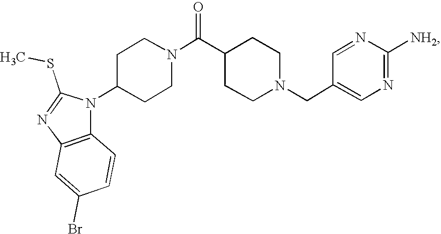 | | | |
| 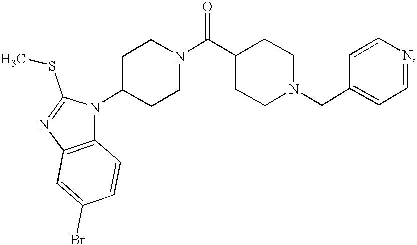 | | | |
| 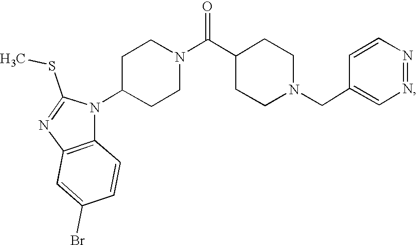 | | | |
| 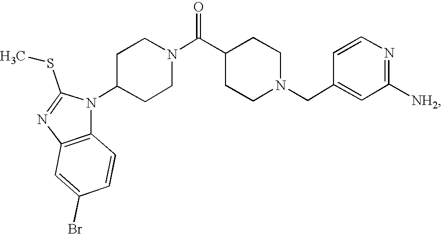 | | | |
| 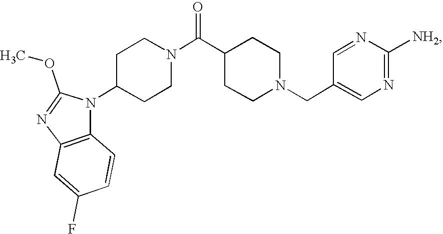 | | | |
| 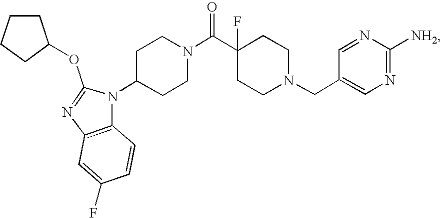 | | | |
| 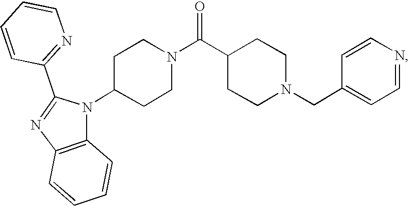 | | | |
| 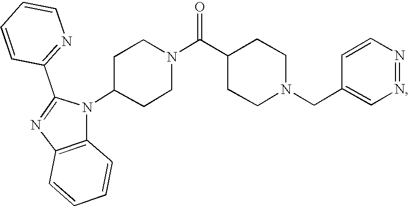 | | | |
| 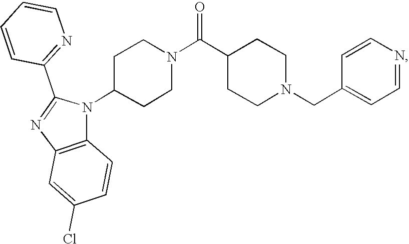 | | | |
| 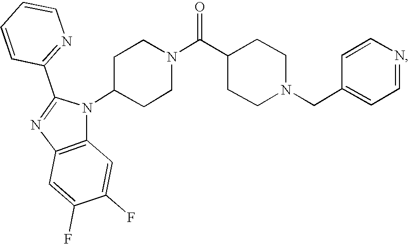 | | | |
| 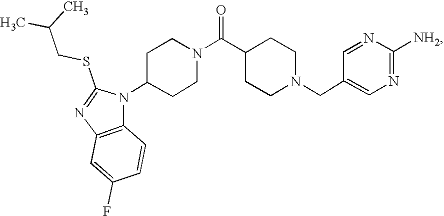 | | | |
| 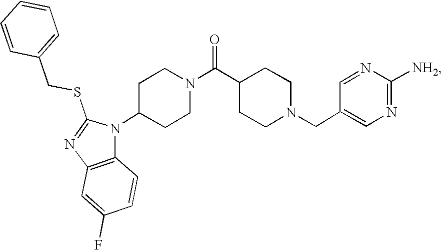 | | | |
| 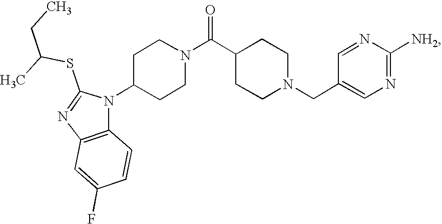 | | | |
| 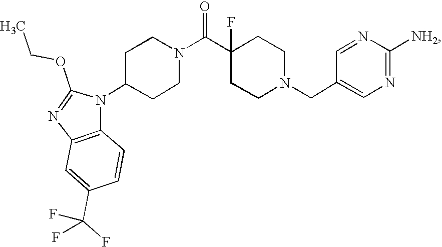 | | | |
| 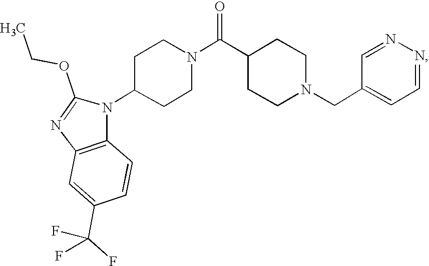 | | | |
| 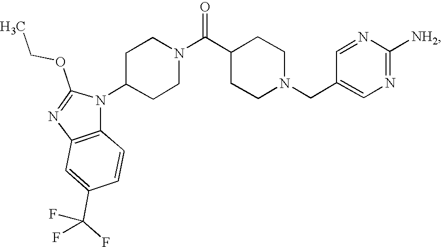 | | | |
| 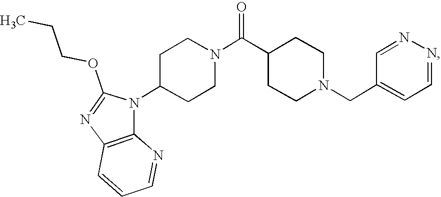 | | | |
| 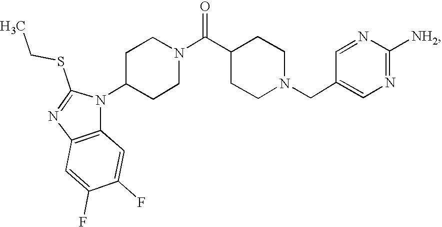 | | | |
| 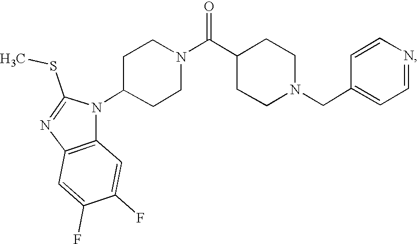 | | | |
| 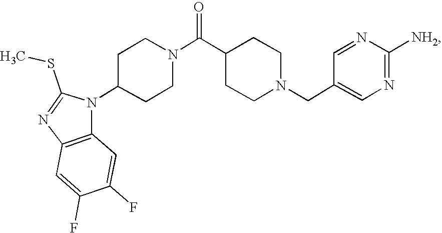 | | | |
| 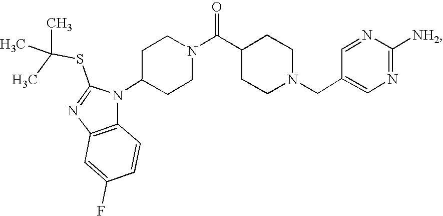 | | | |
| 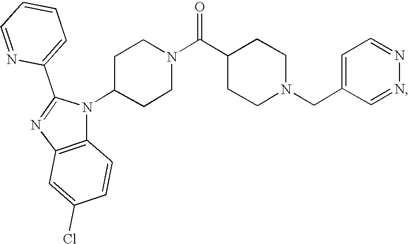 | | | |
| 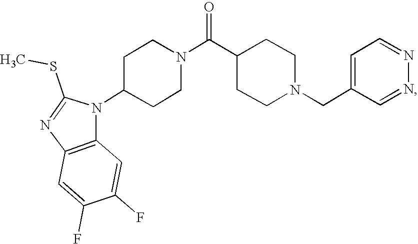 | | | |
| 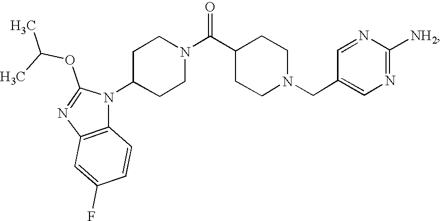 | | | |
| 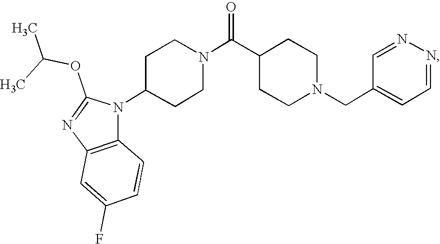 | | | |
| 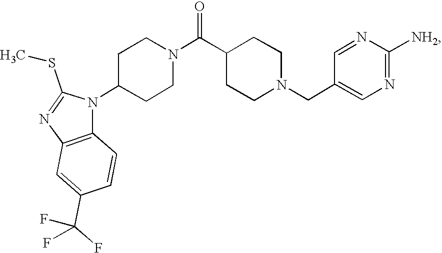 | | | |
| 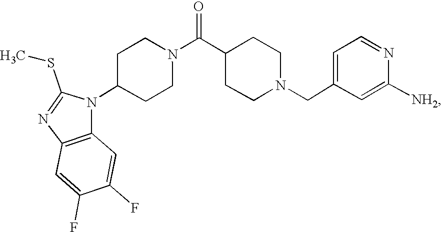 | | | |
| 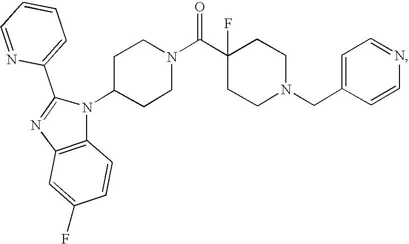 | | | |
| 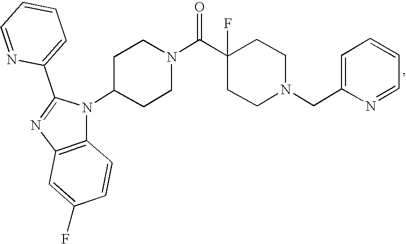 | | | |
| 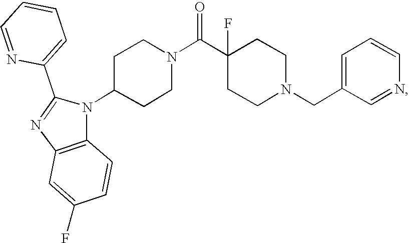 | | | |
| 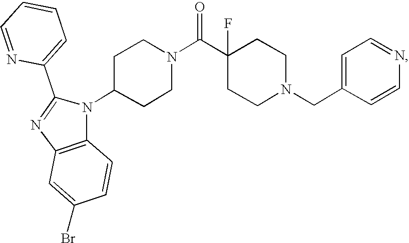 | | | |
| 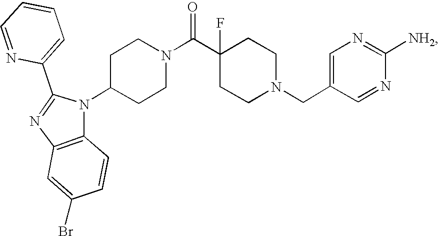 | | | |
| 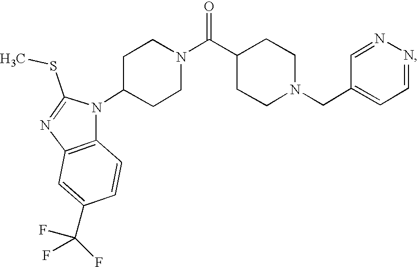 | | | |
| 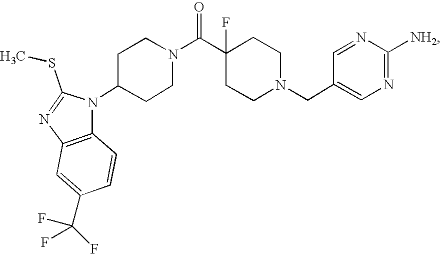 | | | |
| 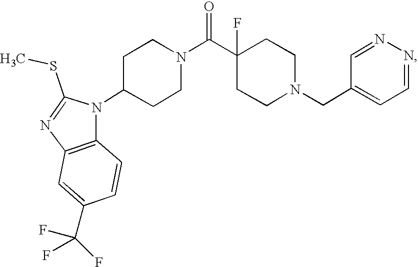 | | | |
| 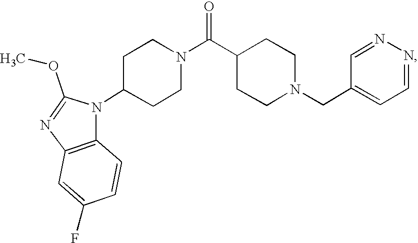 | | | |
| 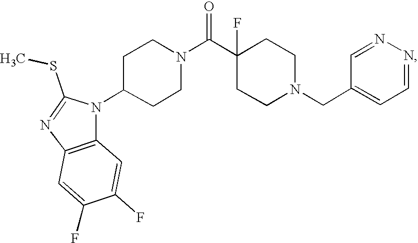 | | | |
| 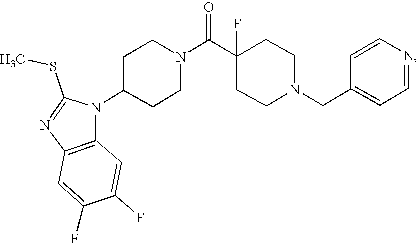 | | | |
| 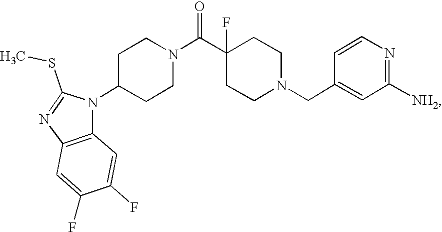 | | | |
| 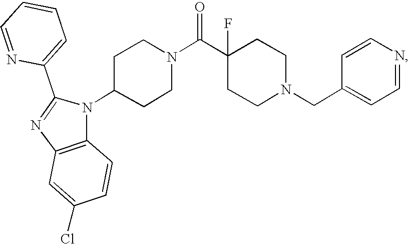 | | | |
| 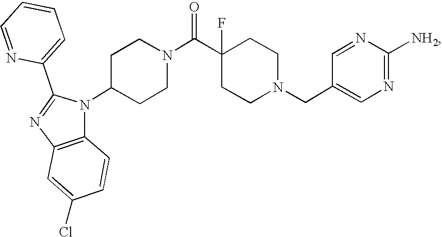 | | | |
| 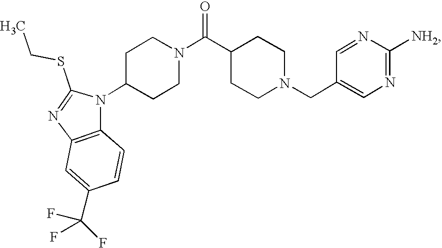 | | | |
| 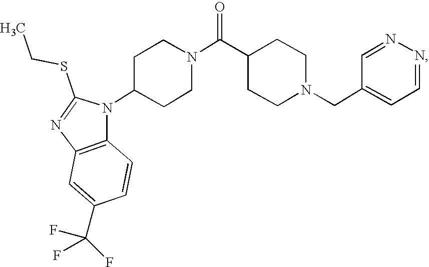 | | | |
| 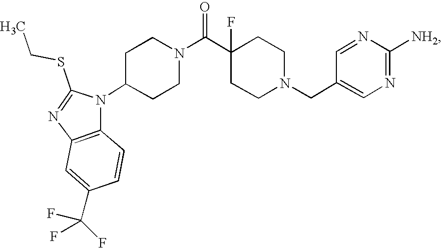 | | | |
| 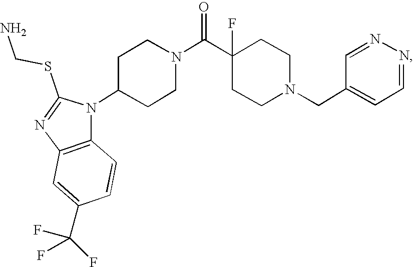 | | | |
| 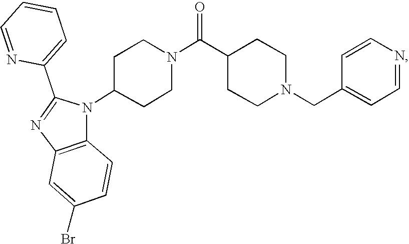 | | | |
| 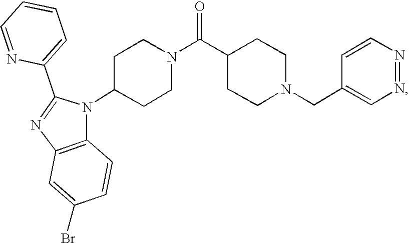 | | | |
| 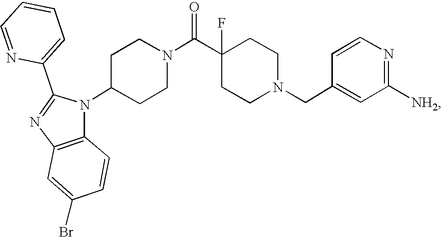 | | | |
| 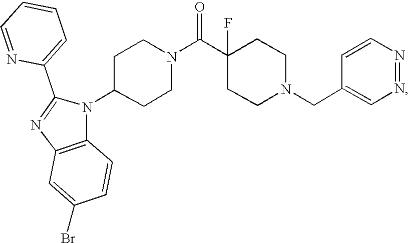 | | | |
| 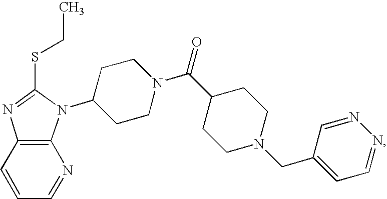 | | | |
| 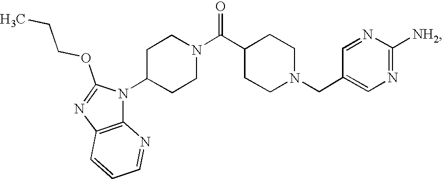 | | | |
| 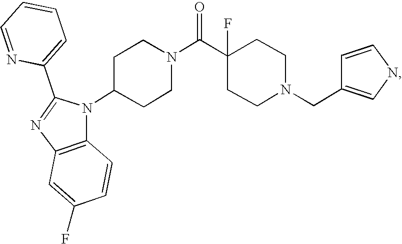 | | | |
| 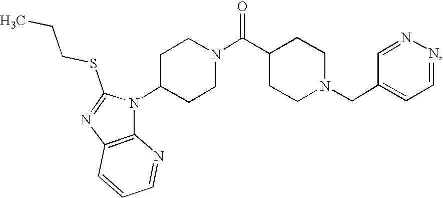 | | | |
| 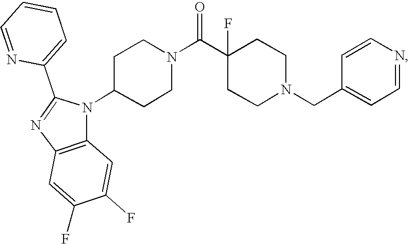 | | | |
| 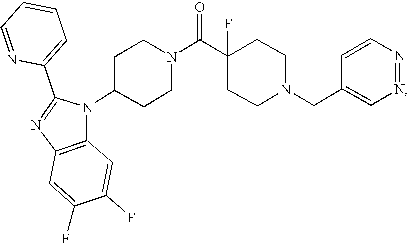 | | | |
| 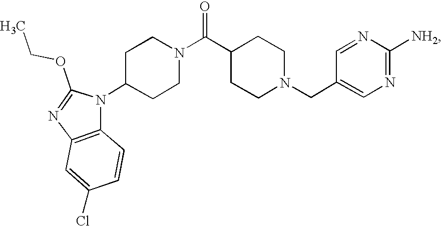 | | | |
| 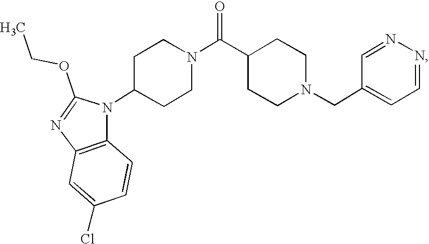 | | | |
| 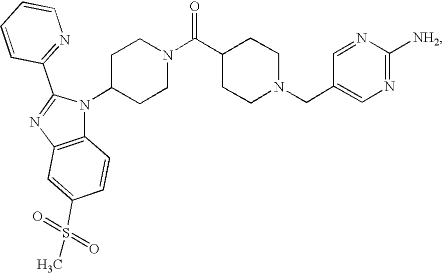 | | | |
| 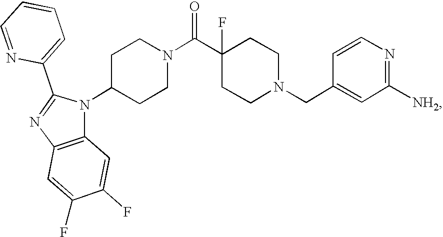 | | | |
| 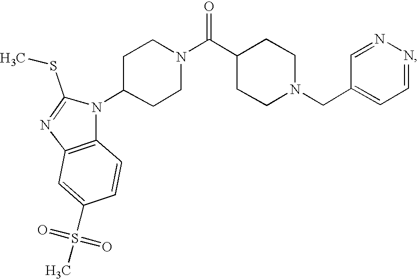 | | | |
| 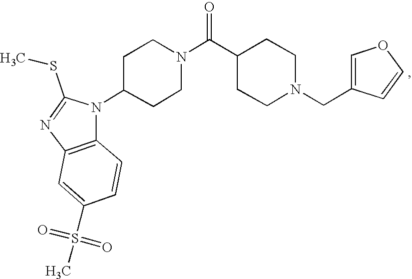 | | | |
| 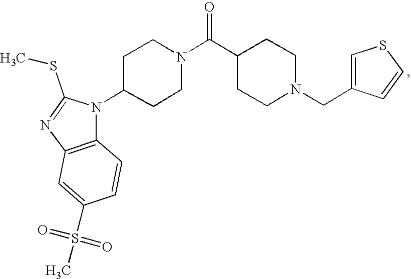 | | | |
| 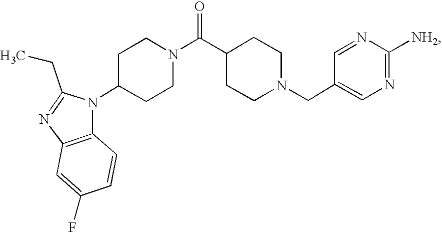 | | | |
| 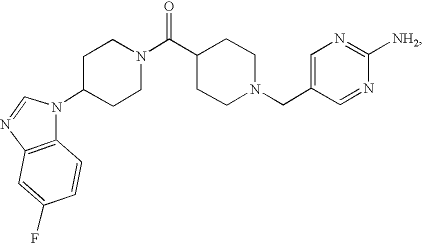 | | | |
| 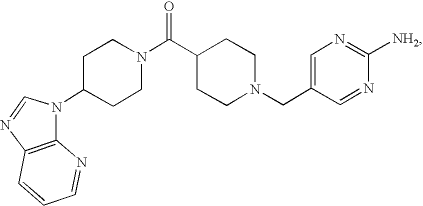 | | | |
| 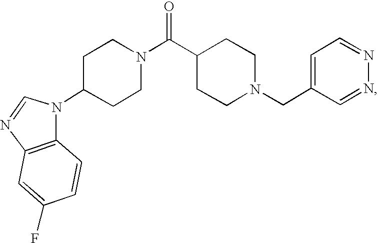 | | | |
| 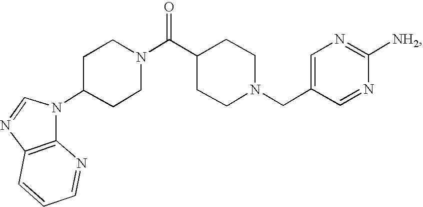 | | | |
| 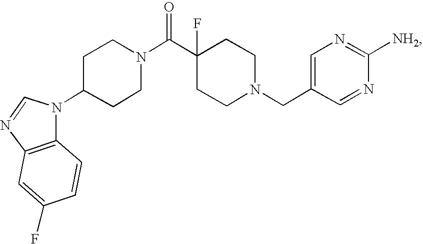 | | | |
| 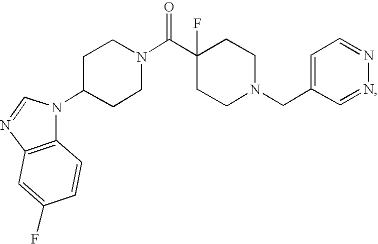 | | | |
| 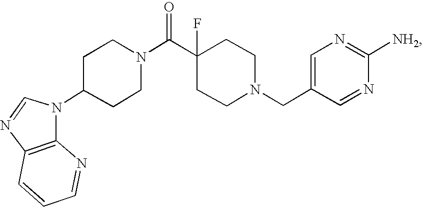 | | | |
| 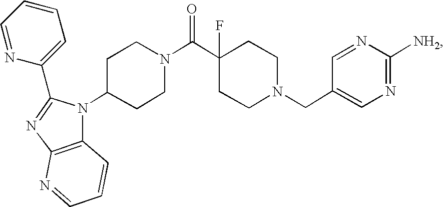 | | | |
| 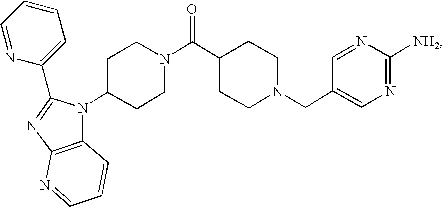 | | | |
| 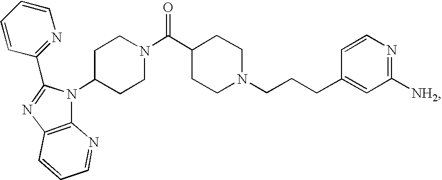 | | | |
| 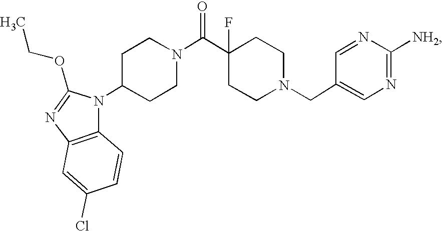 | | | |
| 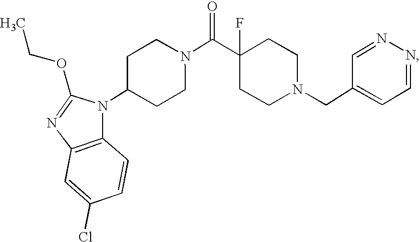 | | | |
| 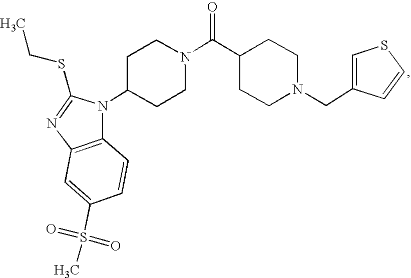 | | | |
| 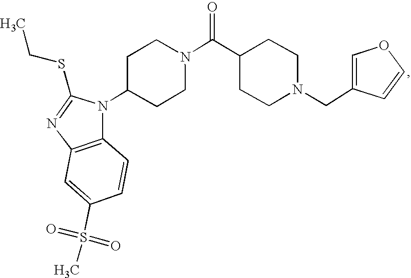 | | | |
| 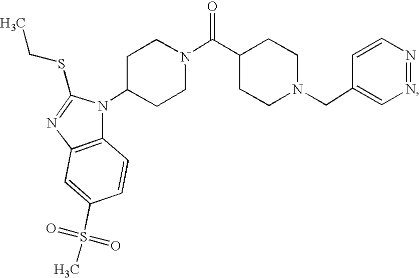 | | | |
| 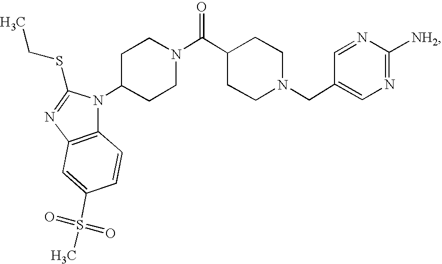 | | | |
| 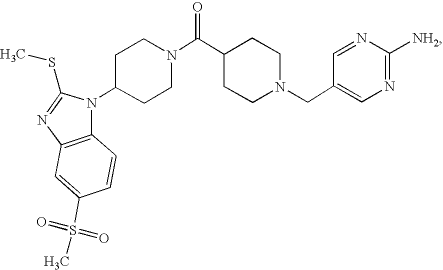 | | | |
| 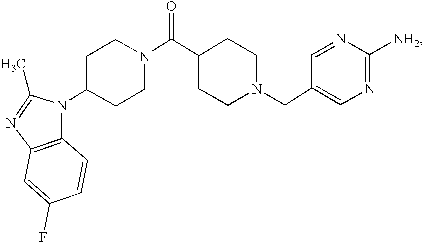 | | | |
| 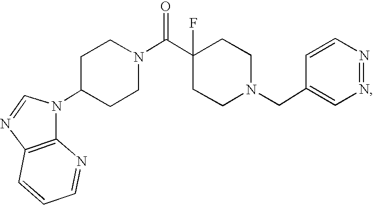 | | | |
| 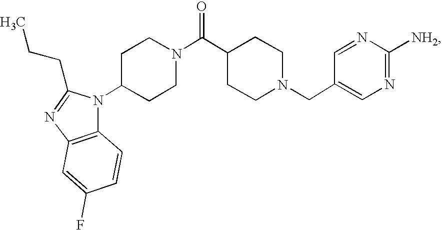 | | | |
| 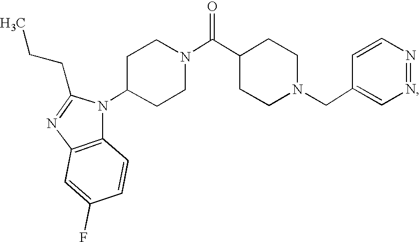 | | | |
| 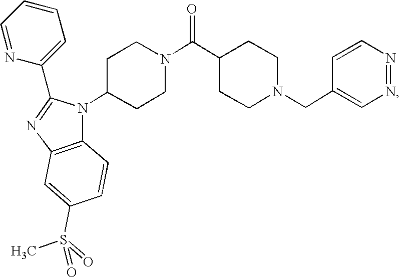 | | | |
| 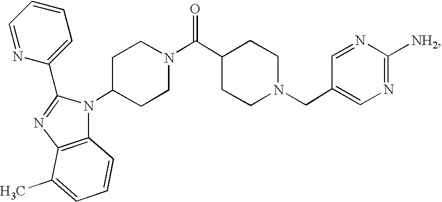 | | | |
| 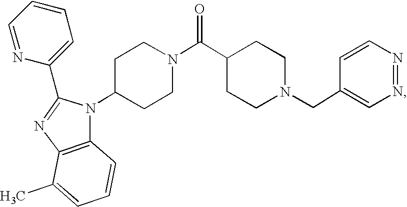 | | | |
| 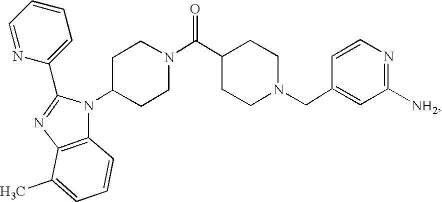 | | | |
| 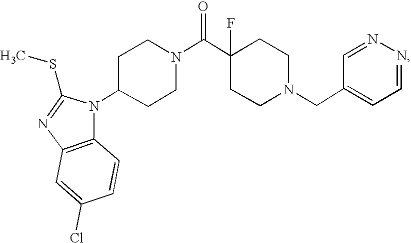 | | | |
| 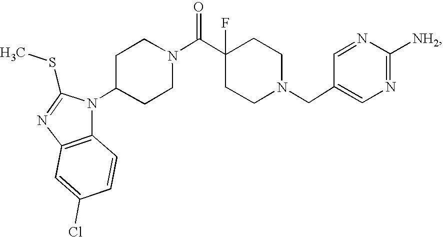 | | | |
| 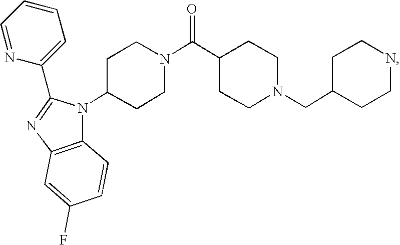 | | | |
| 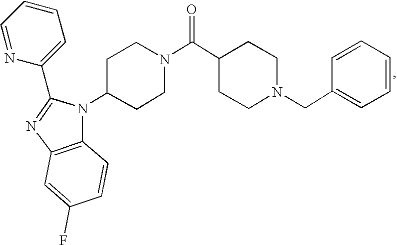 | | | |
| 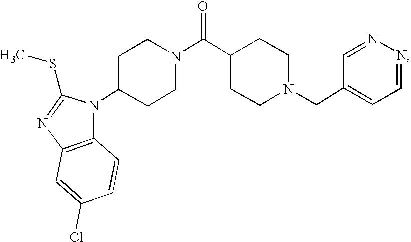 | | | |
| 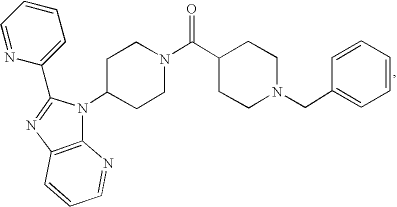 | | | |
| 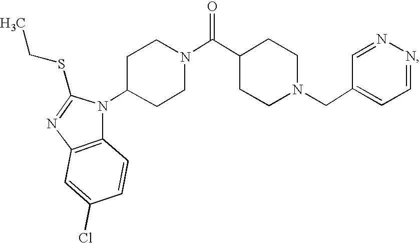 | | | |
| 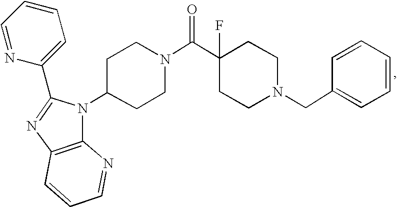 | | | |
| 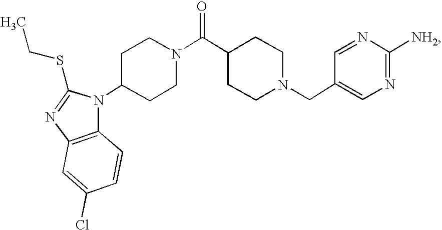 | | | |
| 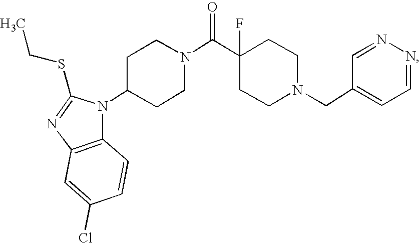 | | | |
| 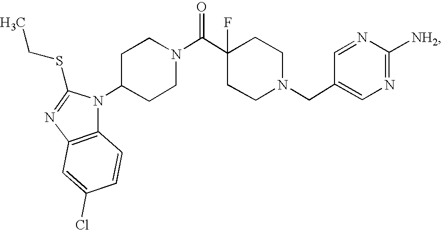 | | | |
| 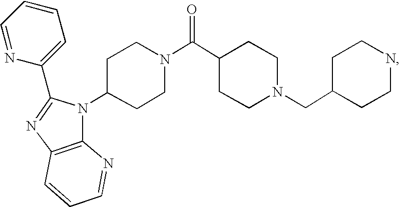 | | | |
| 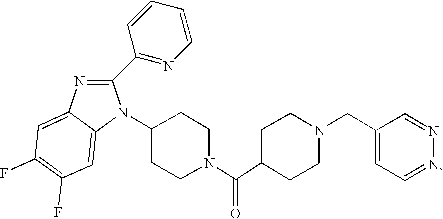 | | | |
| 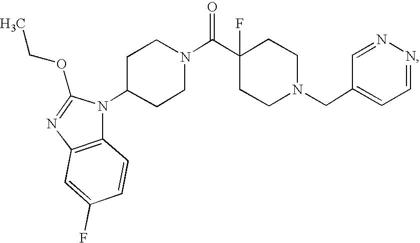 | | | |
| 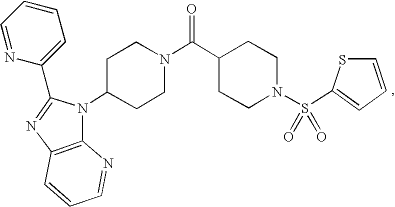 | | | |
| 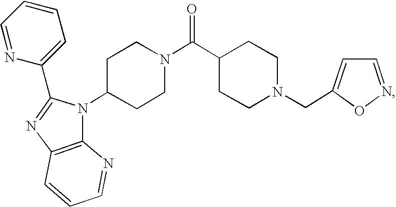 | | | |
| 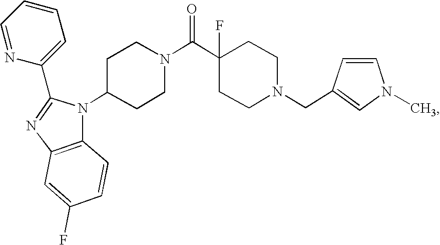 | | | |
| 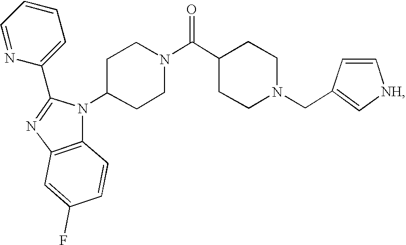 | | | |
| 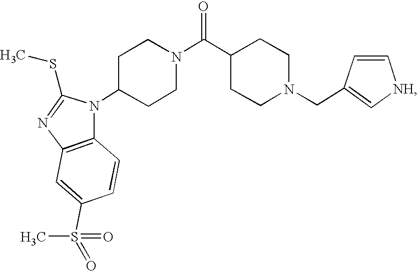 | | | |
| 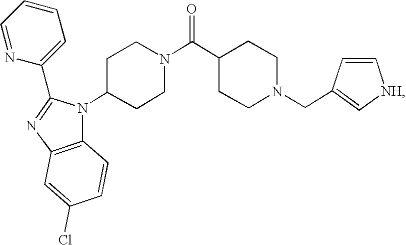 | | | |
| 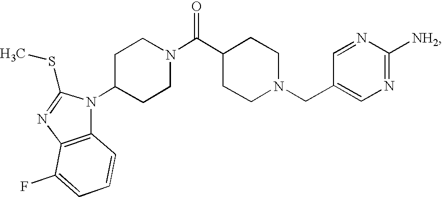 | | | |
| 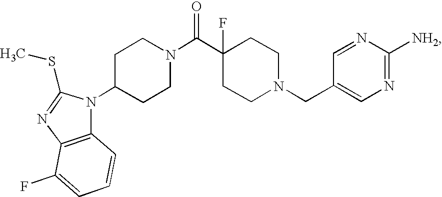 | | | |
| 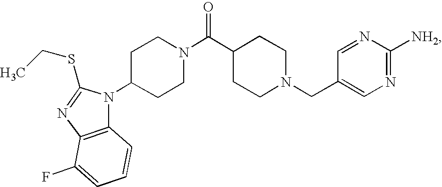 | | | |
| 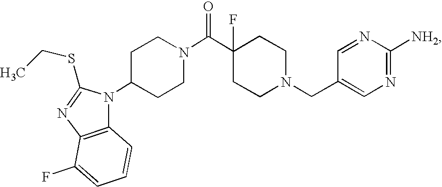 | | | |
| 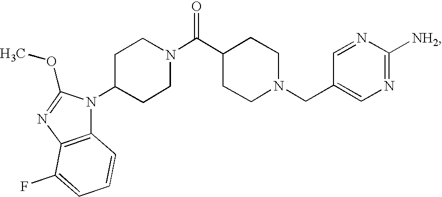 | | | |
| 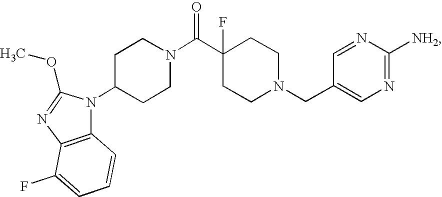 | | | |
| 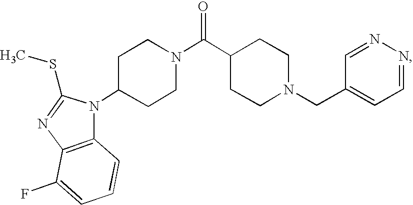 | | | |
| 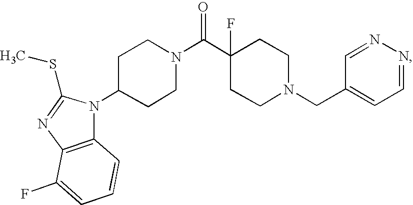 | | | |
| 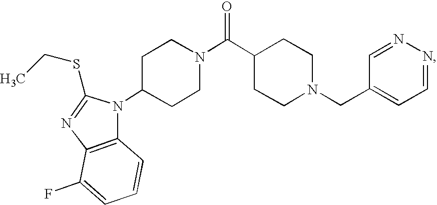 | | | |
| 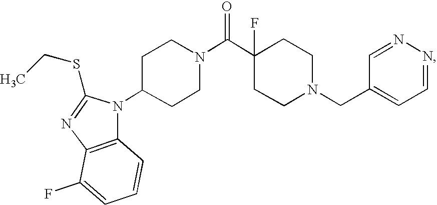 | | | |
| 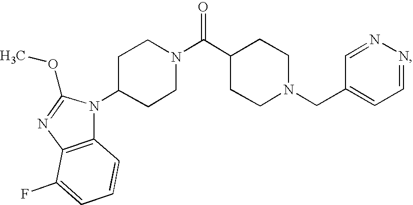 | | | |
| 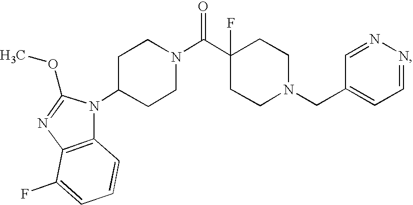 | | | |
| 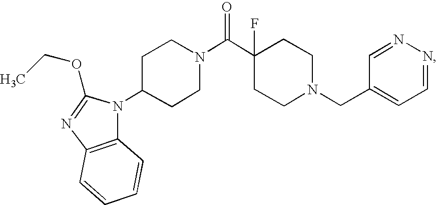 | | | |
| 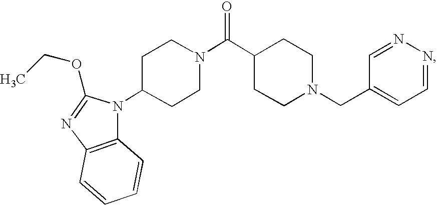 | | | |
| 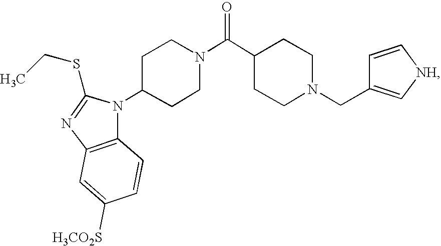 | | | |
| 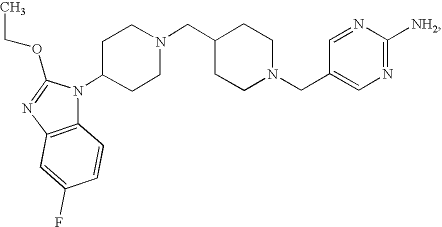 | | | |
| 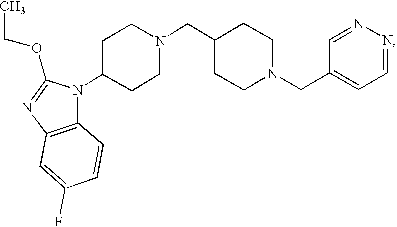 | | | |
| 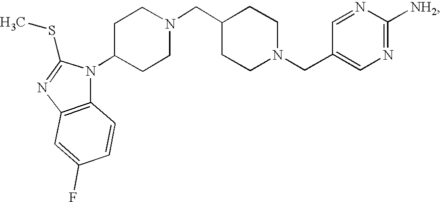 | | | |
| 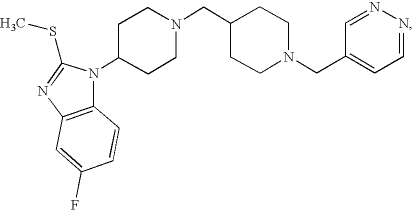 | | | |
| 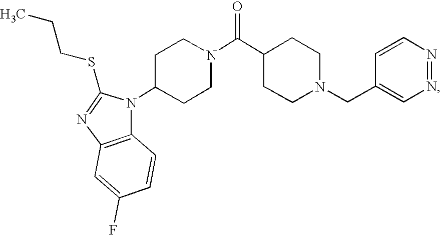 | | | |
| 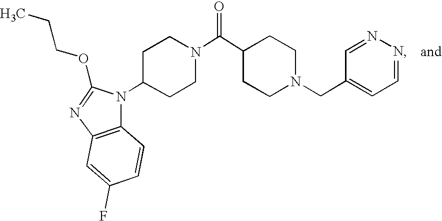 | | | |
| 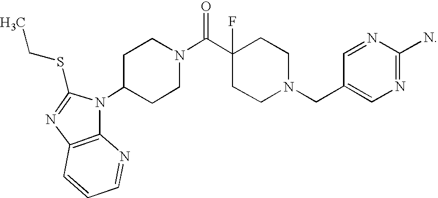 | | | |

In one embodiment, the H₃ antagonists/inverse agonists of the present invention can have a structure according to Formula (IV): 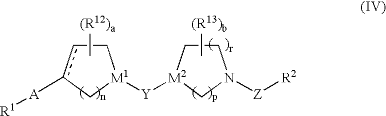
as described in U.S. 2004/0048843, which is herein incorporated by reference in its entirety. Non-limiting examples of compounds of Formula (IV) include: 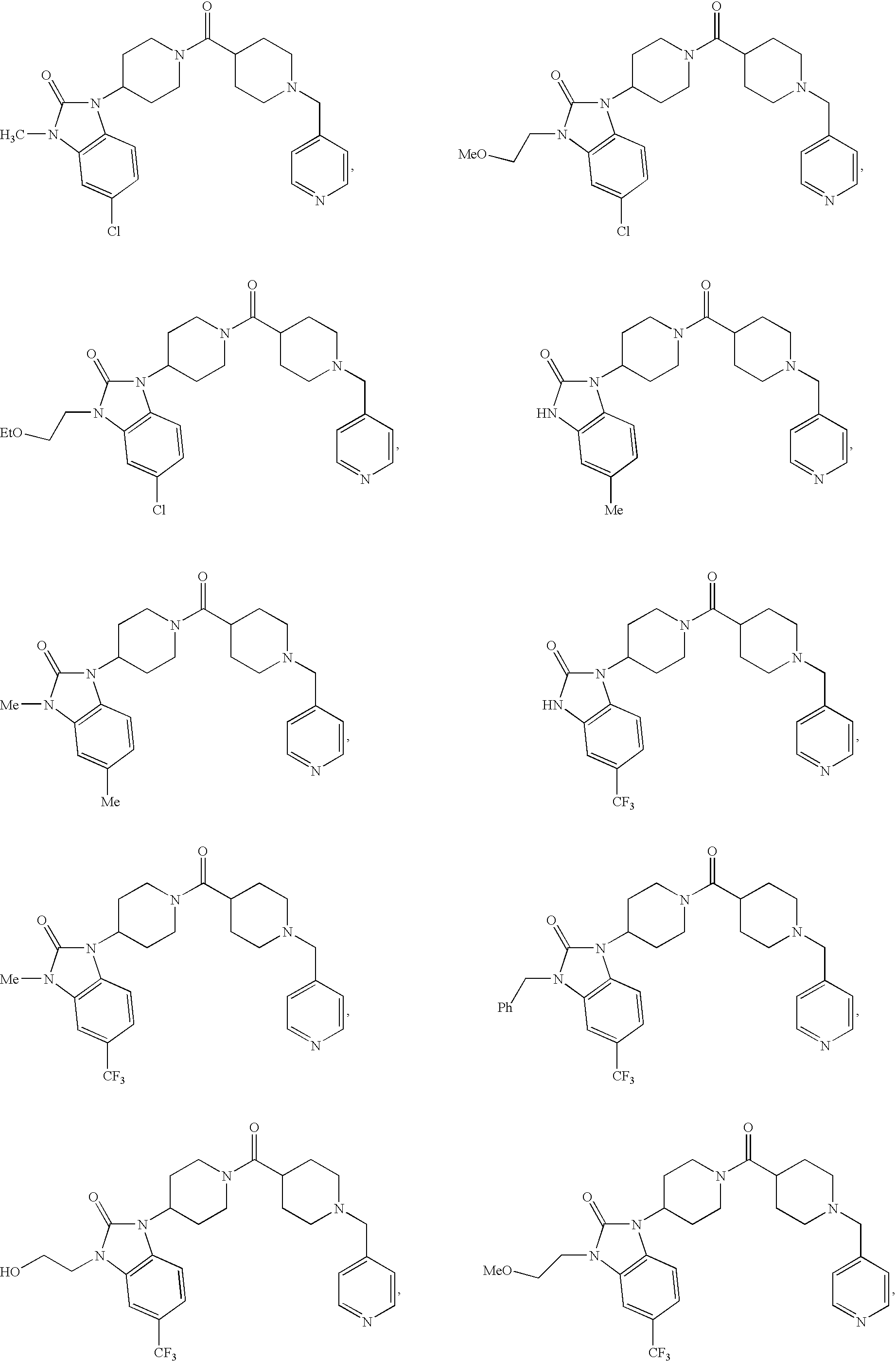 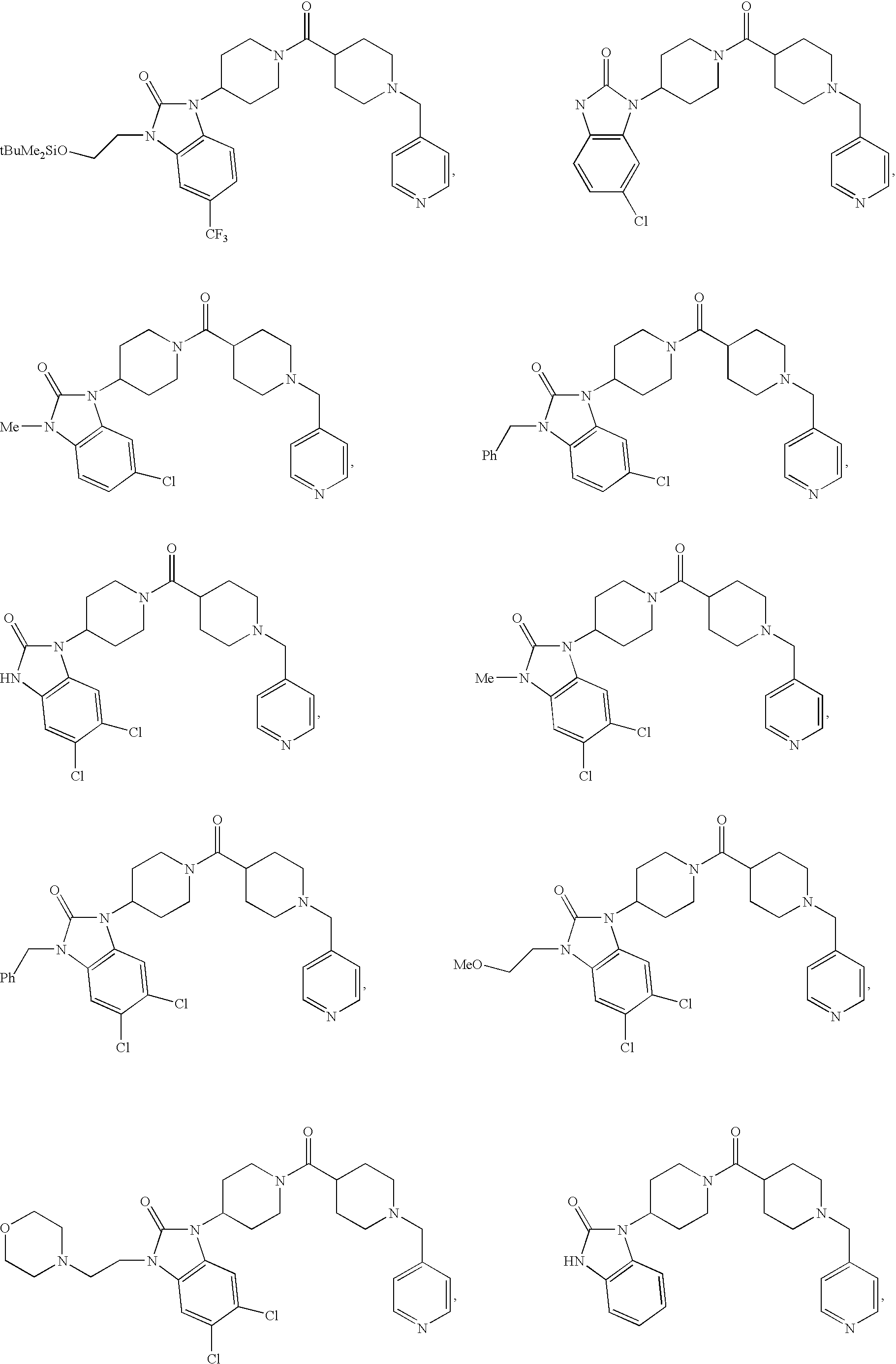 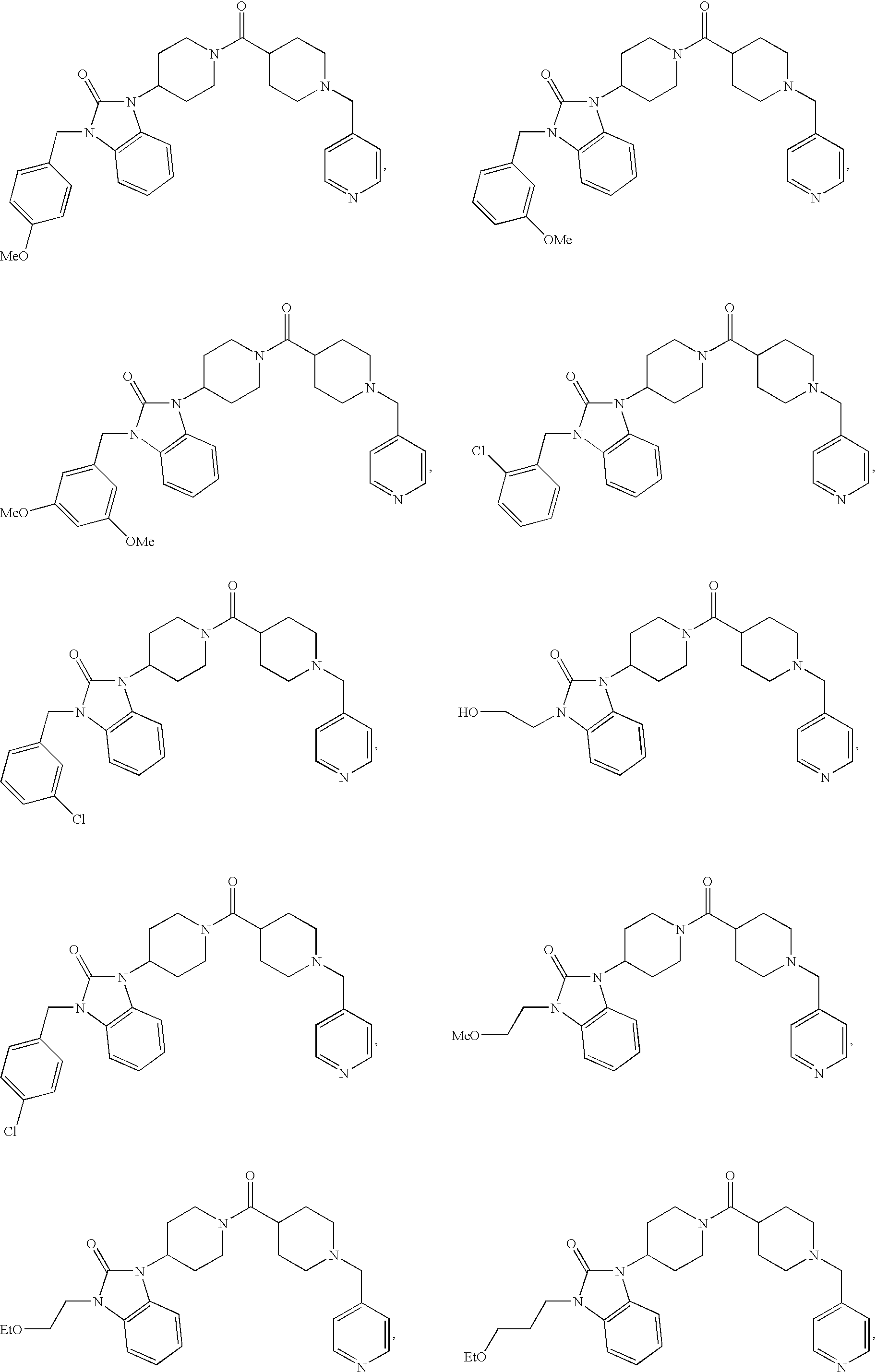 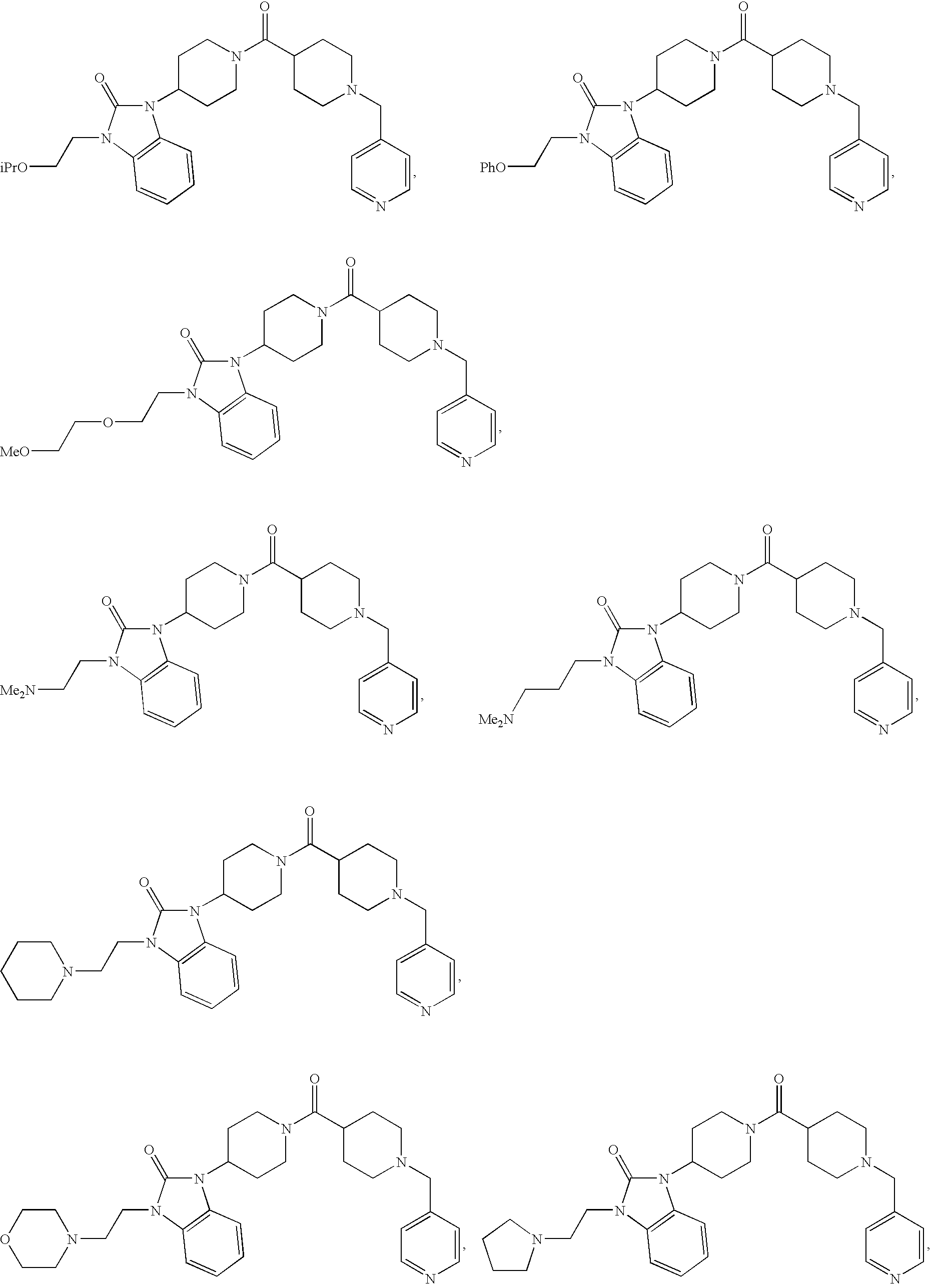 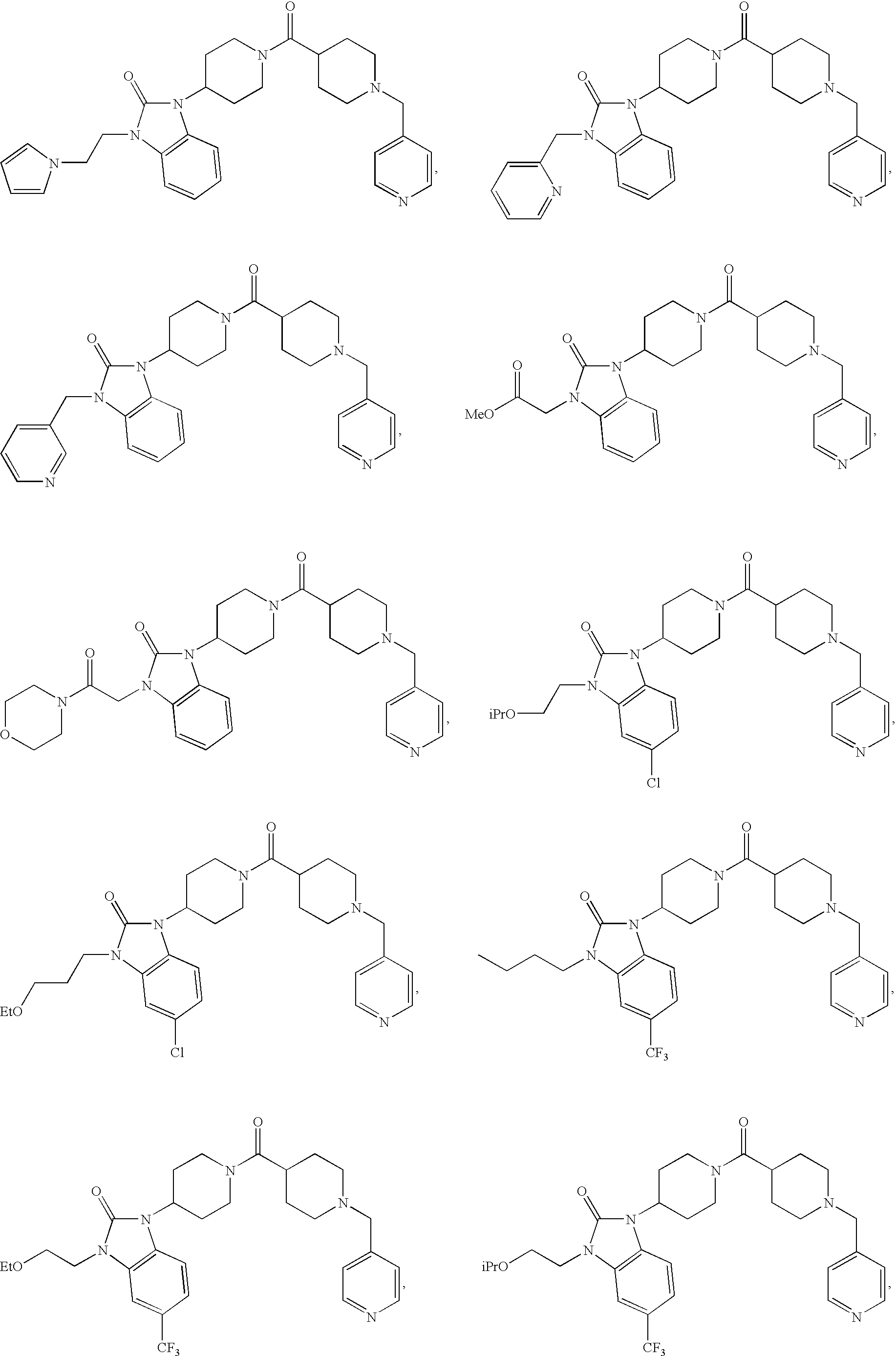 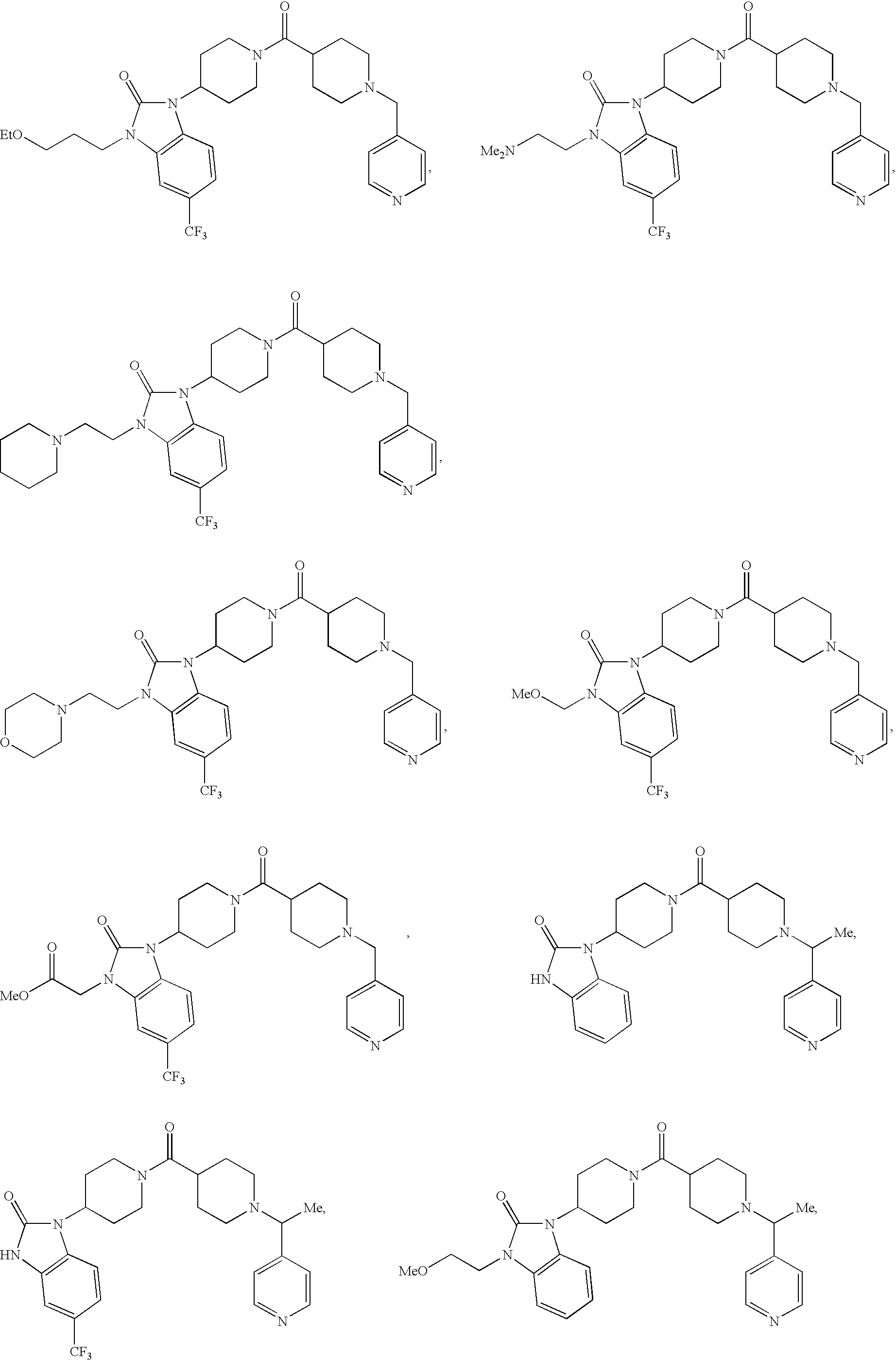 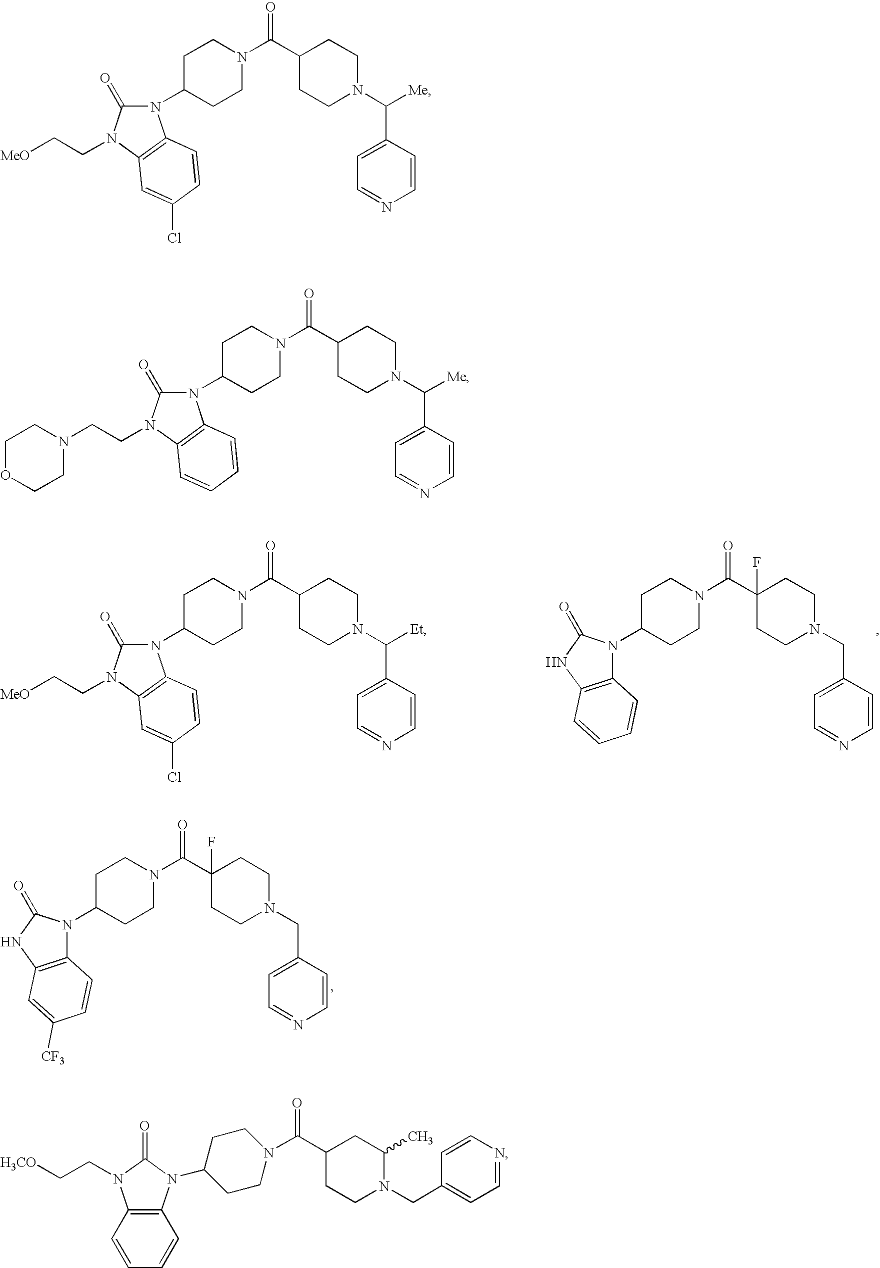 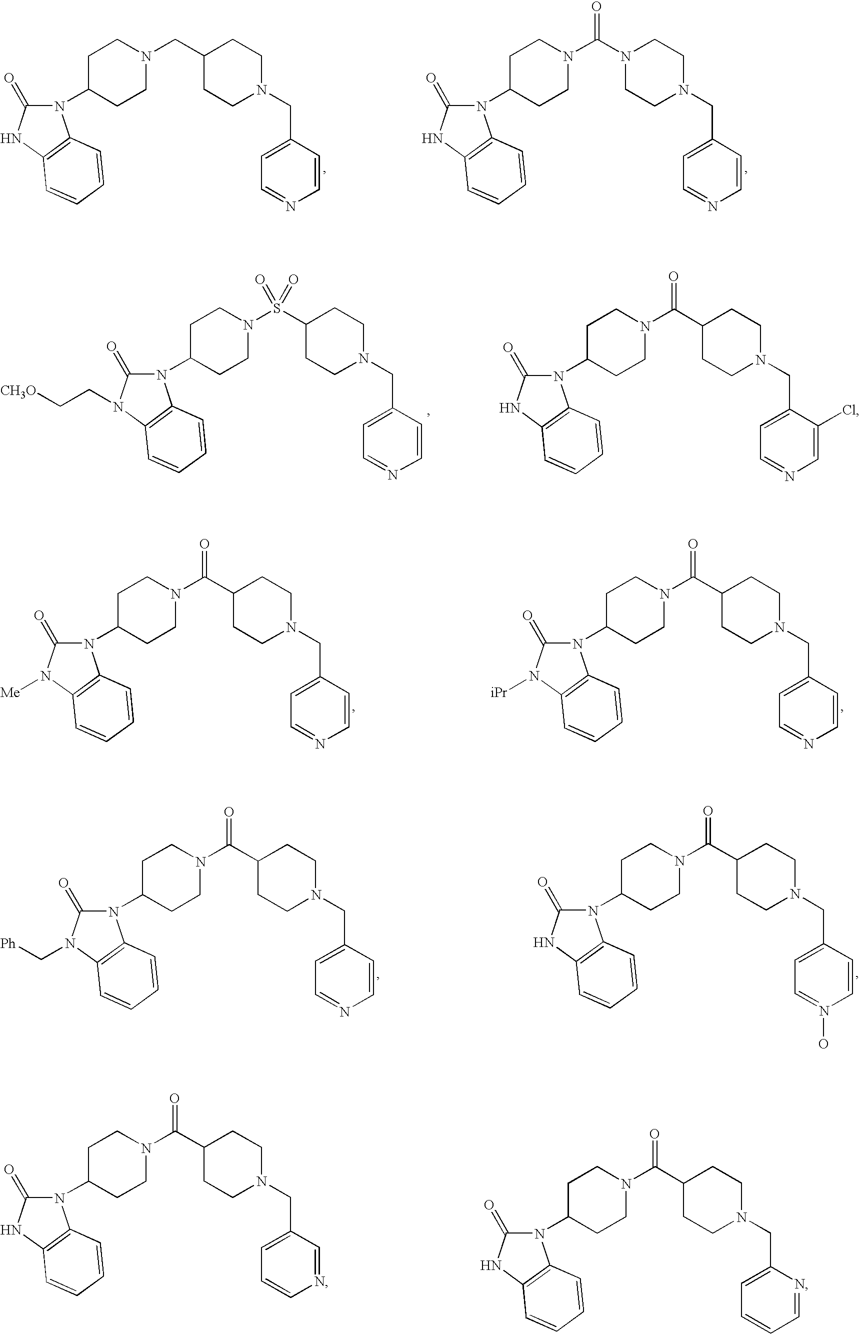 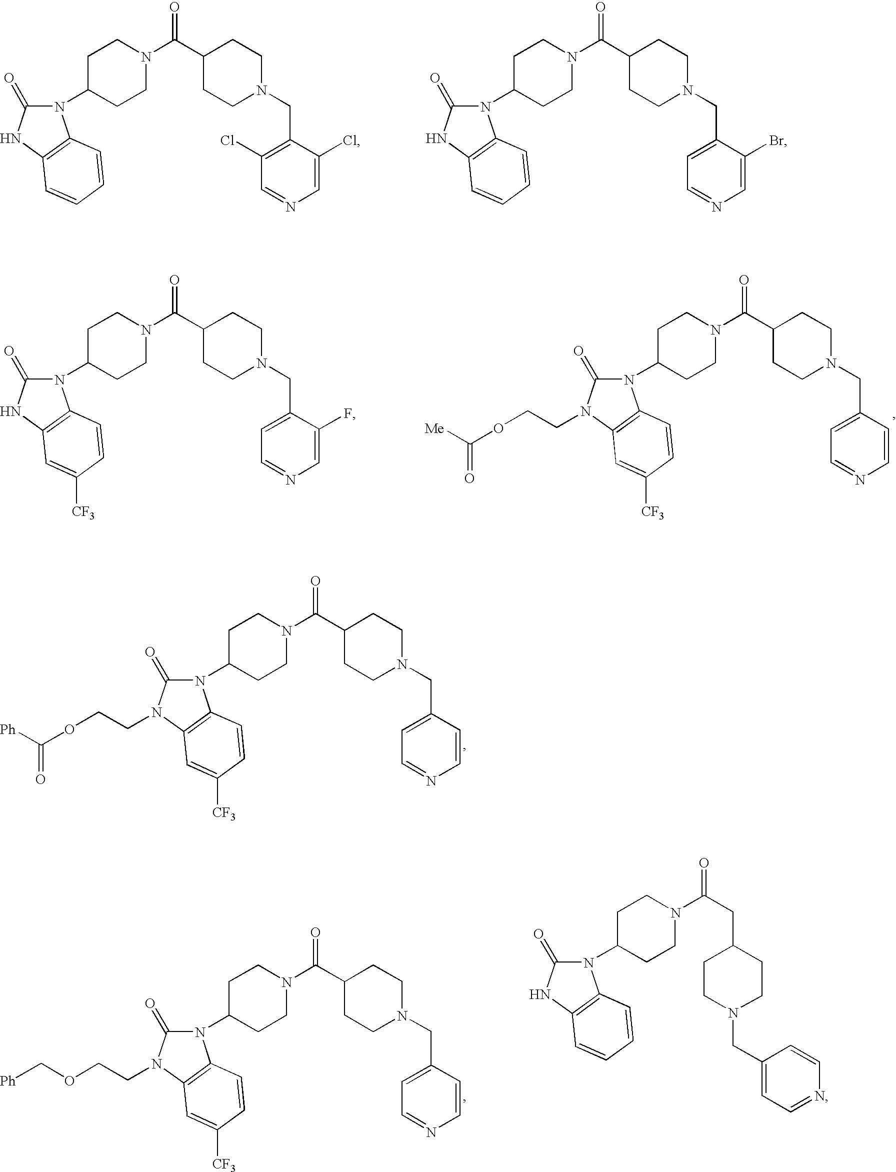 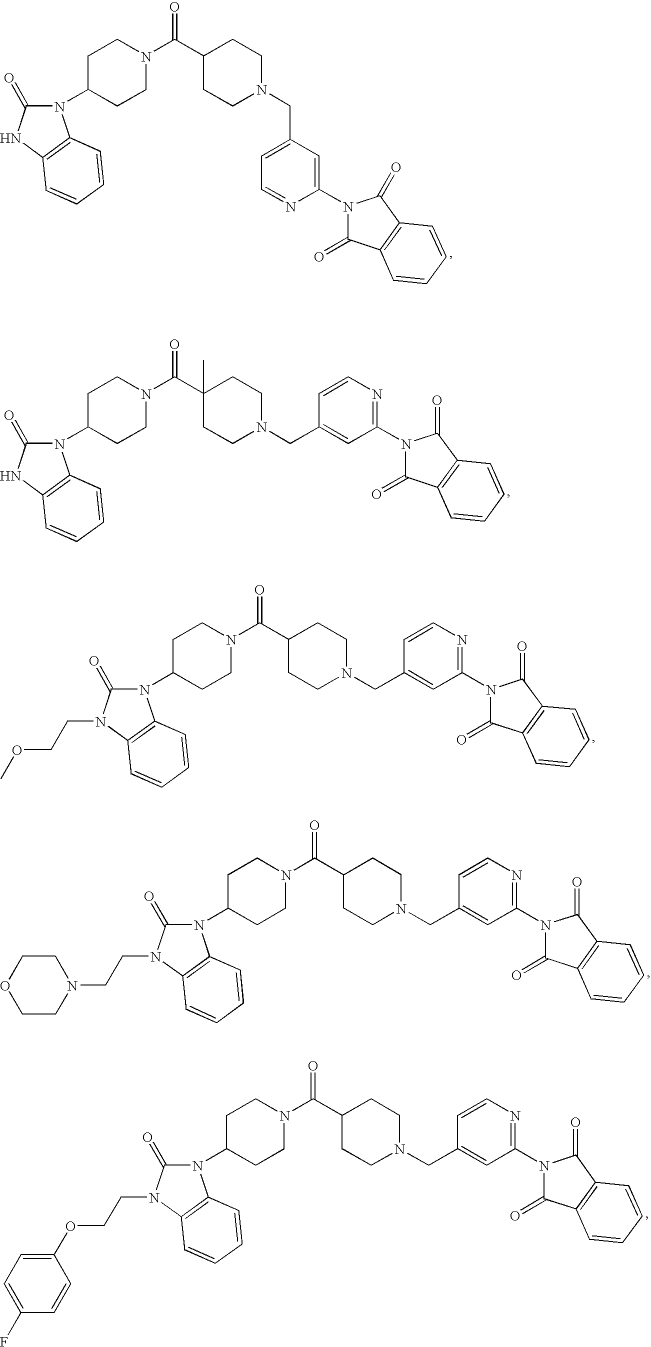 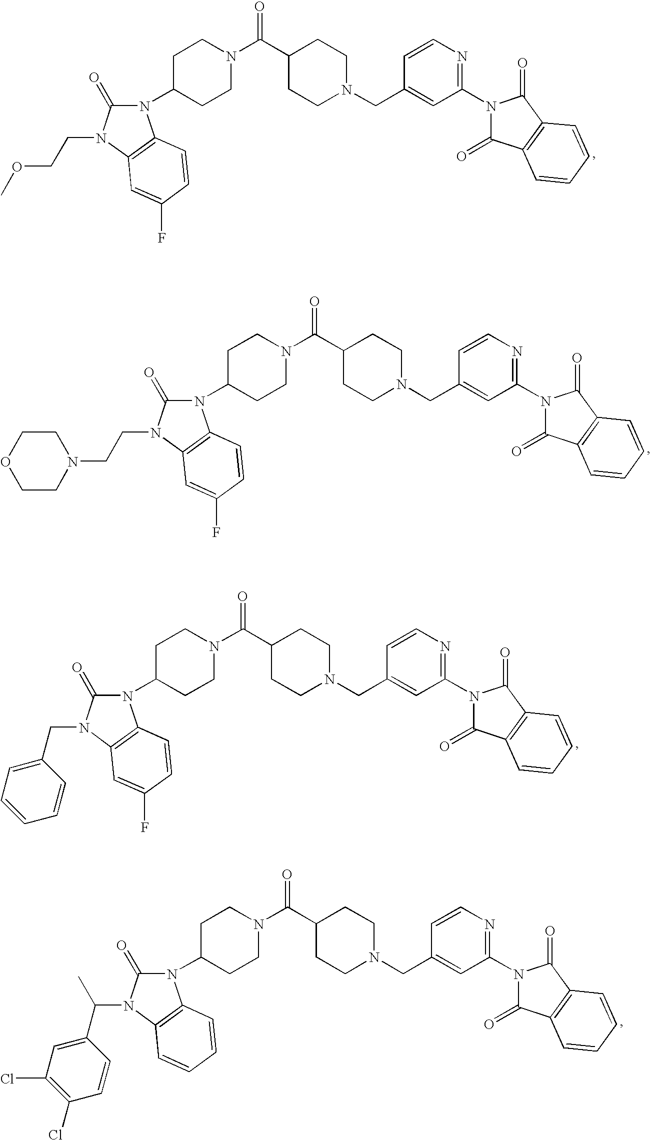 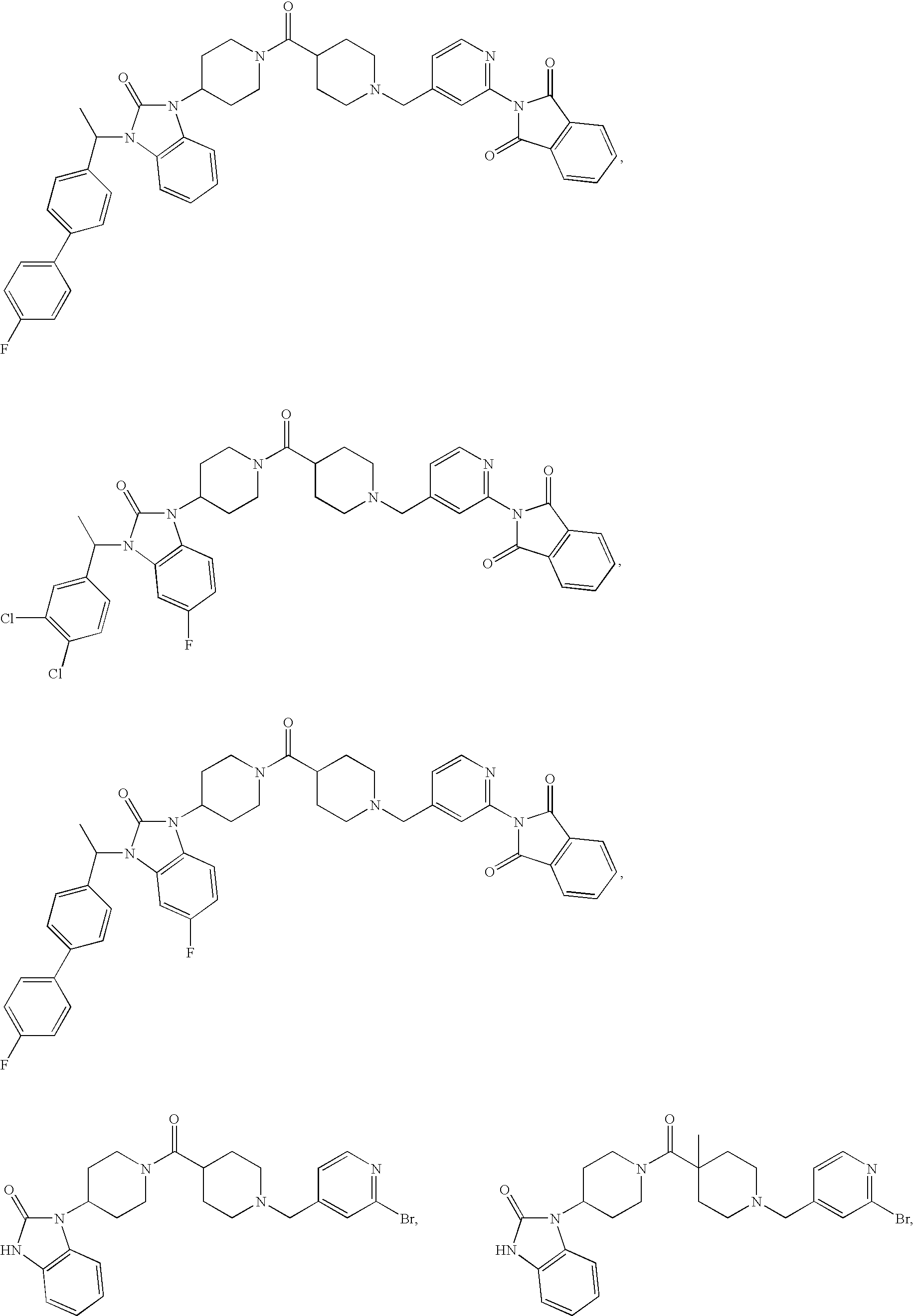 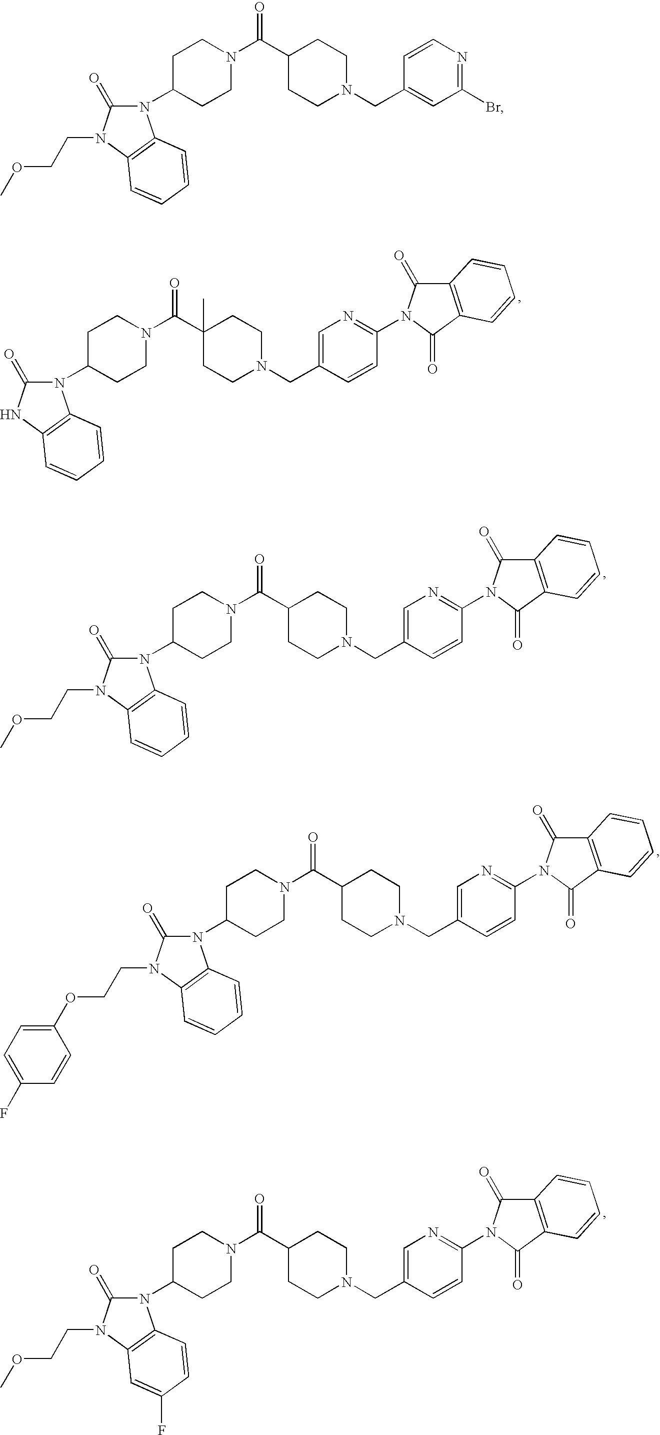 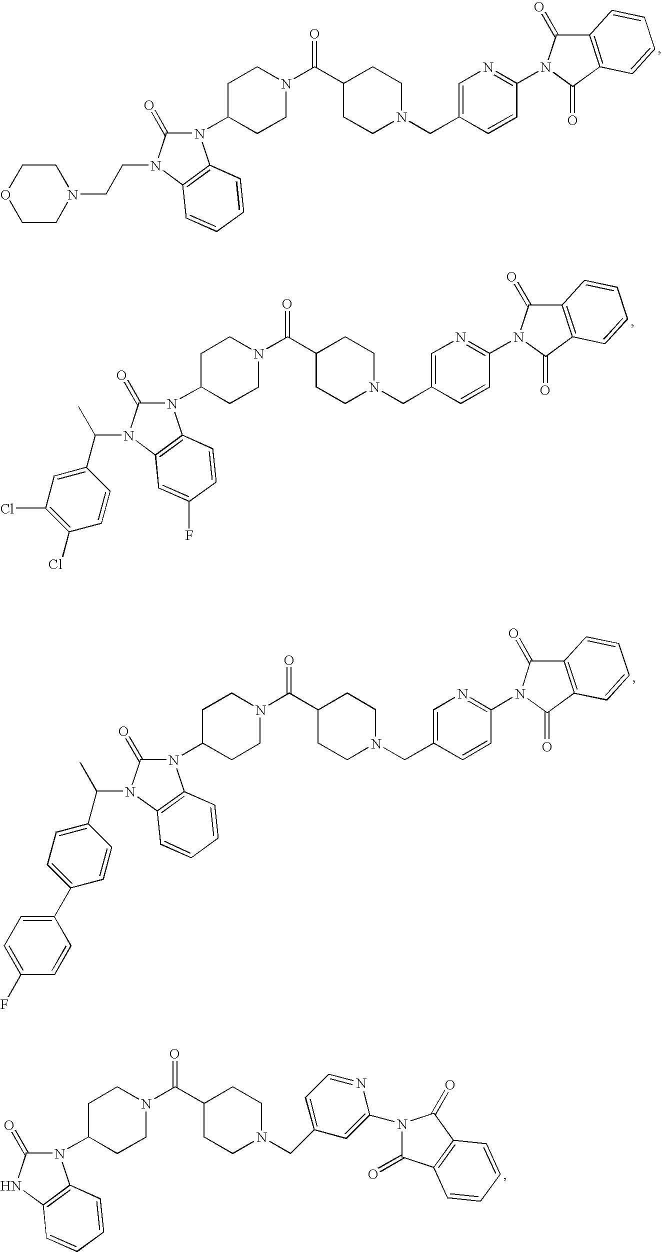 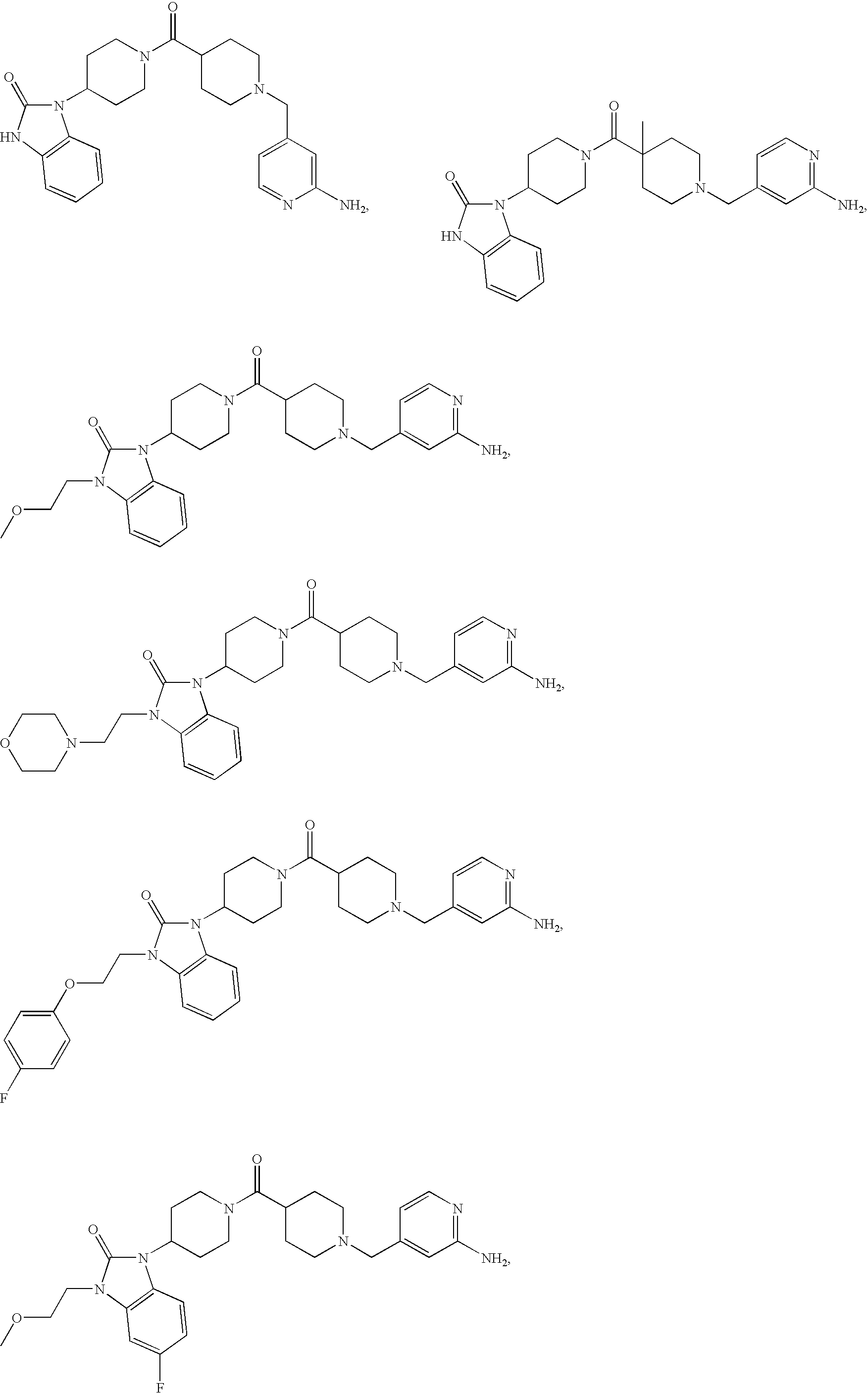 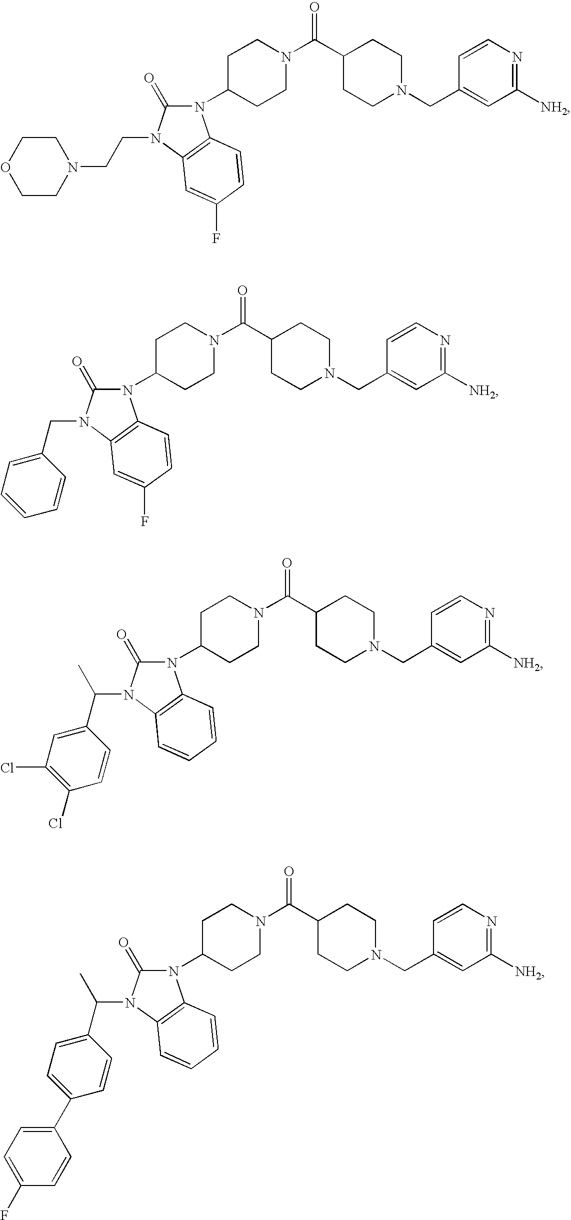 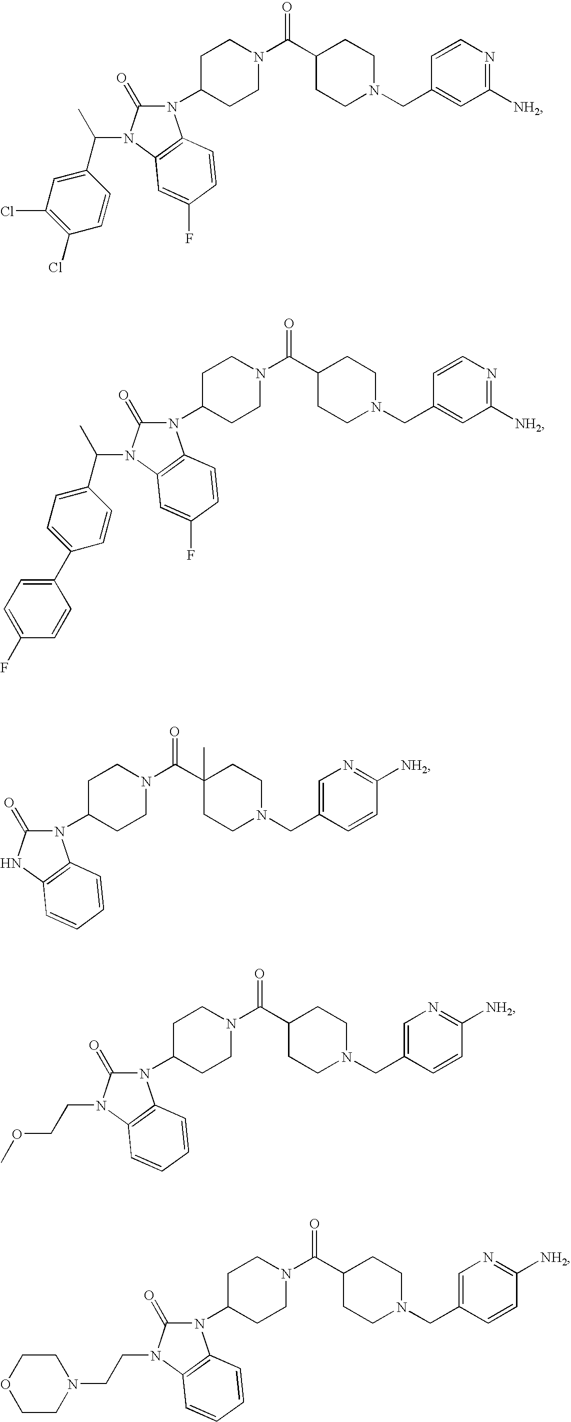 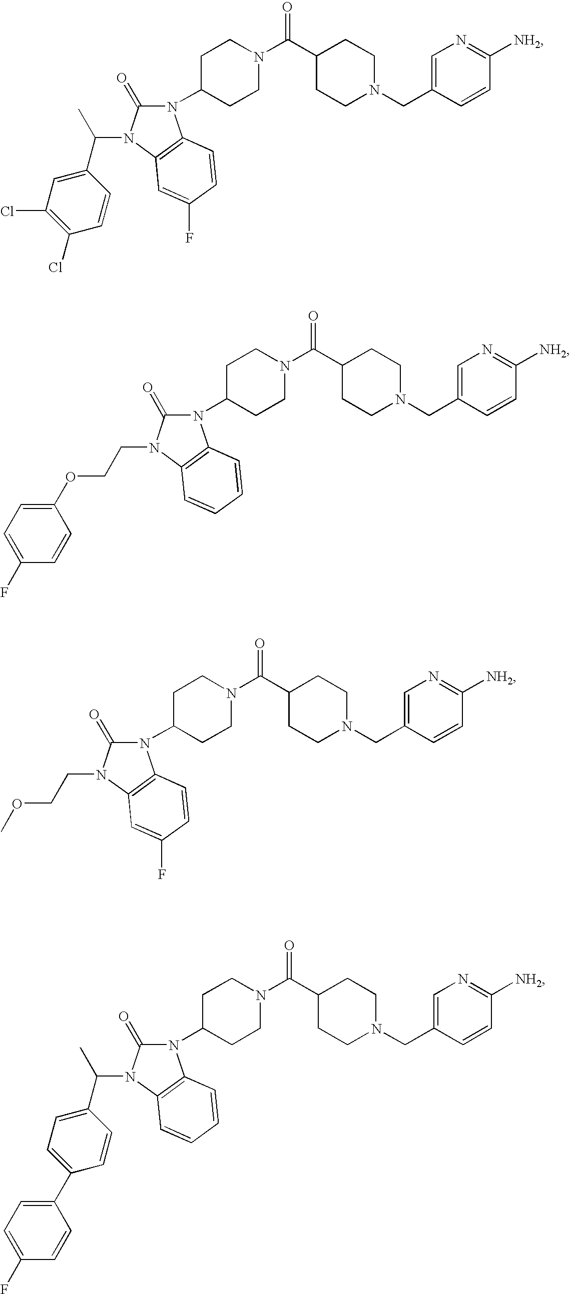 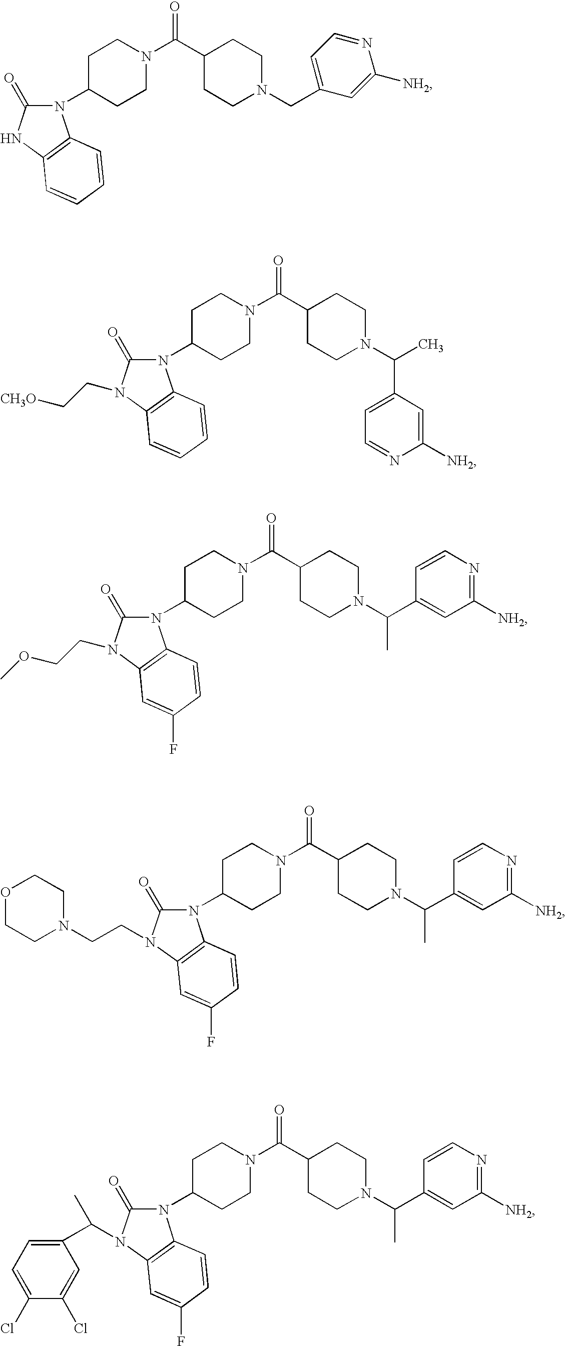 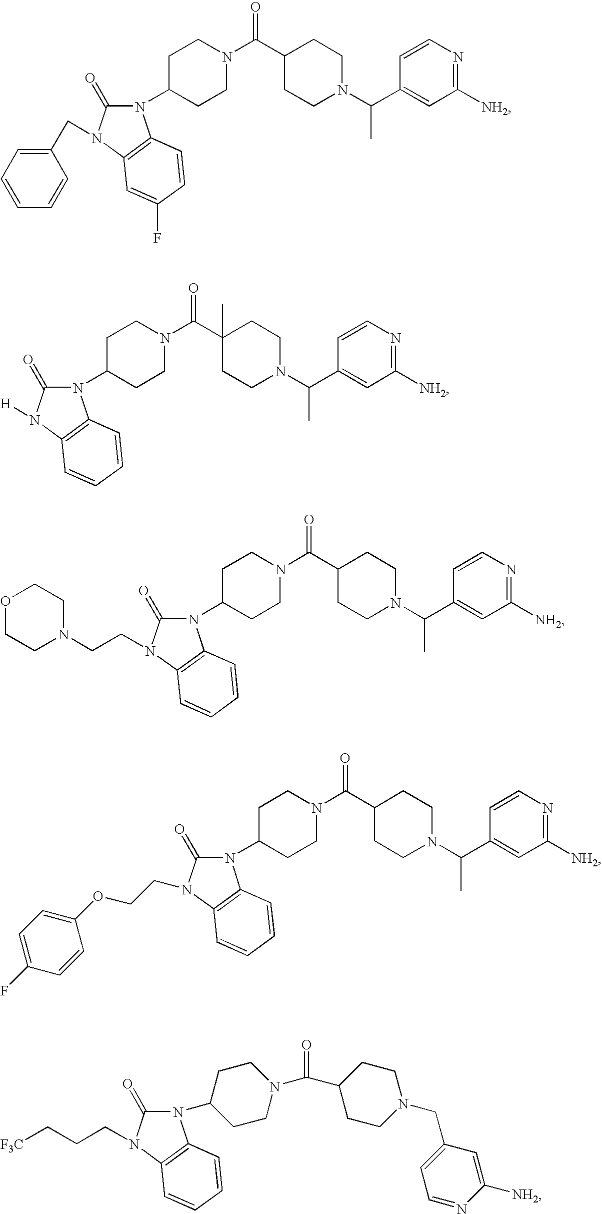 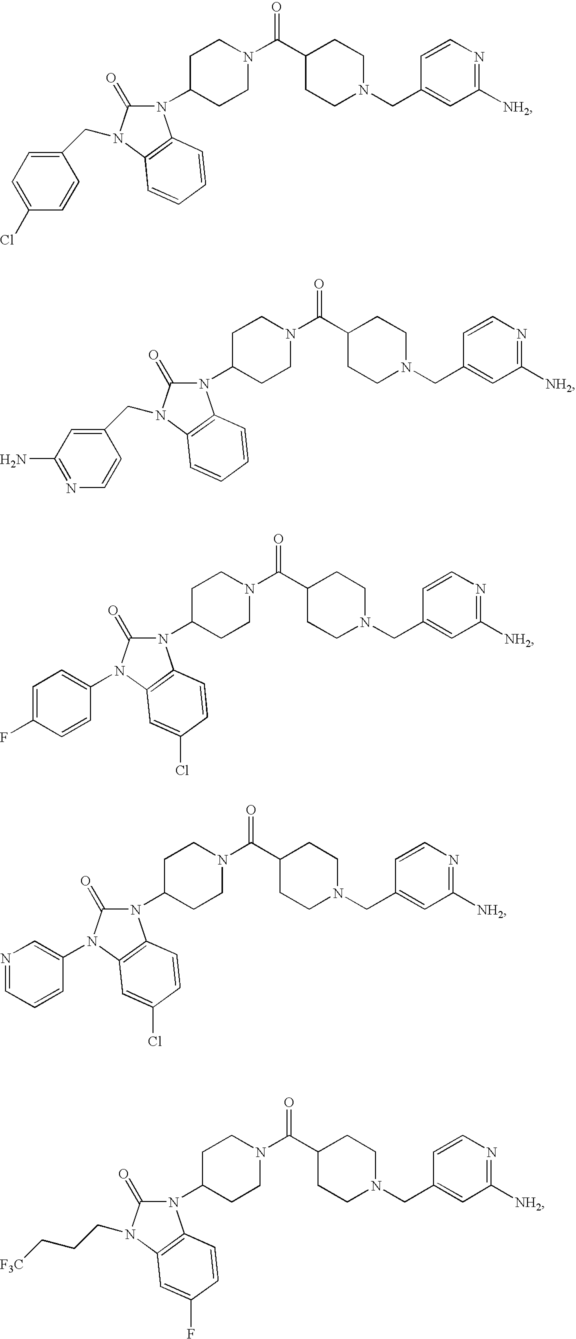 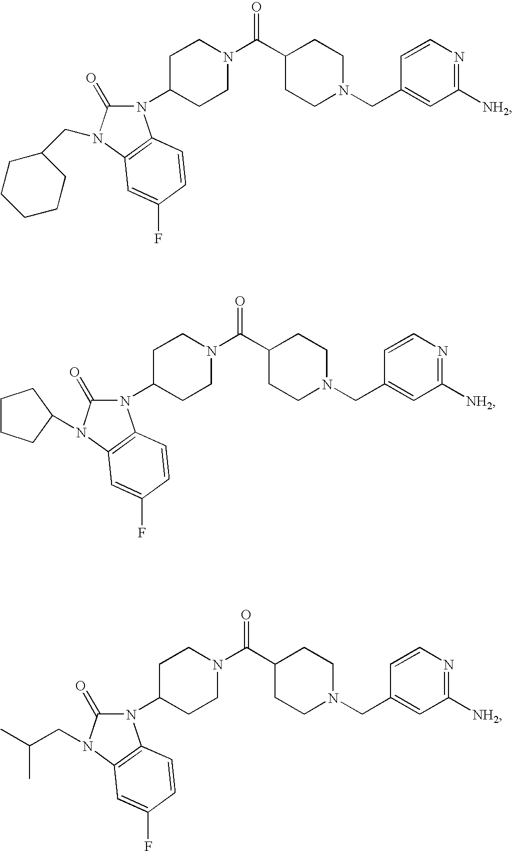
compounds of the following general formula:

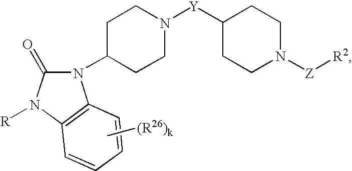

wherein R, (R²⁶)ₖ, Y, Z, and R² are as defined in the following Table:

| R | (R²⁶)ₖ | Y | Z | R² |
|---|---|---|---|---|
| H | H | —C(O)— | —CH₂— | 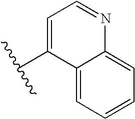 |
| 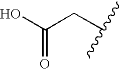 | H | —C(O)— | —CH₂— | 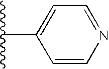 |
| CH₃(CH₂)₃— | H | —C(O)— | —CH(CH₃)— | 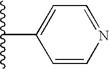 |
| CH₃(CH₂)₃— | H | —C(O)— | —CH(CH₃)— | 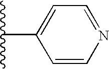 |
| CH₃O(CH₂)₂— | H | —C(O)— | —CH(CN)— | 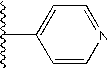 |
| CH₃O(CH₂)₂— | H | —C(O)— | —C(CH₃)₂— | 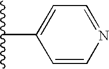 |
| H | H | —C(O)— | bond | 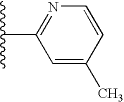 |
| CH₃O(CH₂)₂— | 5-F | —C(O)— | —CH₂— | 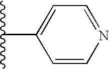 |
| CH₃O(CH₂)₂— | 5-F | —C(O)— | —CH(CH₃)— | 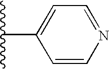 |
| 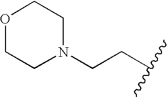 | 5-F | —C(O)— | —CH₂— | 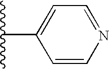 |
| 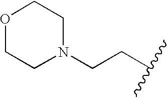 | 5-F | —C(O)— | —CH(CH₃)— | 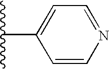 |
| H | 5-F | —C(O)— | —CH₂— | 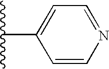 |
| H | 5-F | —C(O)— | —CH(CH₃)— | 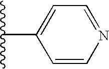 |
| H | 5-Cl | —C(O)— | —CH₂— | 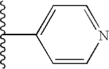 |
| CH₃— | 5-F | —C(O)— | —CH₂— | 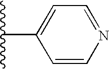 |
| C₆H₅—CH₂— | 5-Cl | —C(O)— | —CH₂— | 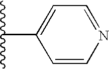 |
| 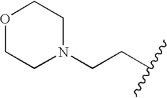 | 5-Cl | —C(O)— | —CH₂— | 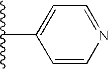 |
| 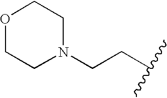 | 5-Cl | —C(O)— | —CH(CH₃)— | 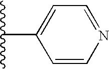 |
| 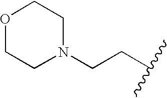 | 5-Cl | —C(O)— | —CH(CH₃)— | 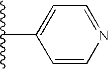 |
| H | 5-CF₃— | —C(O)— | —CH₂— | 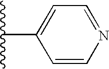 |
| H | H | C(O)CH₂— | —CH₂— | 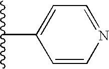 |
| CH₃O(CH₂)₂— | H | C(O)CH₂— | bond | 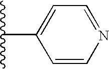 |
| CH₃O(CH₂)₂— | H | C(O)CH₂— | bond | 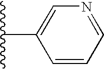 |
| H | 5-CF₃ | —C(O)NH— | —CH₂— | 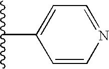 |
| H | 5-CF₃ | —SO₂— | —CH₂— | 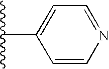 |
| H | 5-CF₃ | C(═N—CN)—NH— | —CH₂— | 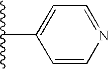 |
| H | H | —C(O)— | bond | 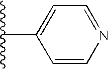 |
| H | H | —C(O)— | —C(O)— | 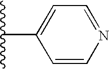 |
| H | H | —C(O)— | —(CH₂)₂— | 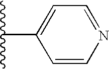 |
| H | H | —C(O)— | —C(O)CH₂— | 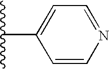 |
| H | 5-CF₃ | —C(O)— | bond | 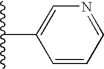 |
| CH₃O(CH₂)₂— | H | —C(O)— | —C(═NH)— | 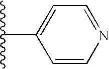 |
| CH₃O(CH₂)₂— | H | —C(O)— | —C(O)— | 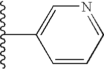 |
| H | 5-CF₃ | —C(O)— | —C(O)— | 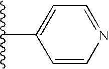 |
| CH₃O(CH₂)₂— | H | —C(O)— | —C(O)NH— | 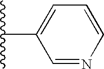 |
| CH₃O(CH₂)₂— | H | —C(O)— | —C(O)— | 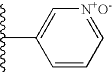 |
| H | 5-CF₃ | —C(O)— | —NH—C(O)— | 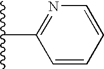 |
| H | 5-CF₃ | —C(O)— | —NH—C(O)— | 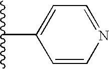 |
| CH₃O(CH₂)₂— | H | —C(O)— | bond | 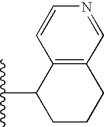 |
| CH₂O(CH₂)₂— | H | —C(O)— | 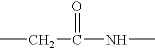 | 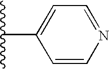 |
| CH₃O(CH₂)₂— | H | —C(O)— | 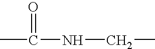 | 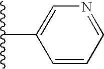 |
| CH₃O(CH₂)₂— | H | —C(O)— | bond | 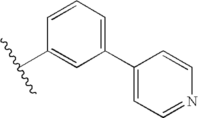 |
| CH₃O(CH₂)₂— | H | —C(O)— | 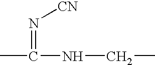 | 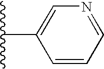 |
| CH₃O(CH₂)₂— | 5-CF₃ | —C(O)— | —(CH₂)₃— | 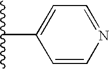 |
| CH₃O(CH₂)₂— | 5-CF₃ | —C(O)— | —(CH₂)₃— | 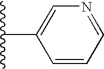 |
| CH₃O(CH₂)₂— | 5-CF₃ | —C(O)— | —C(O)—(CH₂)₂— | 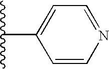 |
| CH₃O(CH₂)₂— | 5-CF₃ | —C(O)— | —(CH₂)₄— | 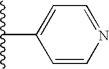 |
| CH₃O(CH₂)₂— | H | —C(O)— | —CH₂— | 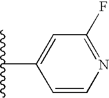 |
| H | 5-CF₃ | —C(O)— | —(CH₂)₄— | 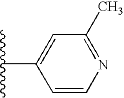 |
| 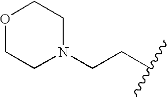 | H | —C(O)— | —CH₂— | 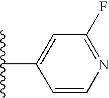 |
| H | H | —C(O)— | —CH(CH₃)— | 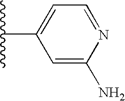 |
| 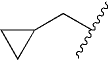 | 5-F | —C(O)— | —CH₂— | 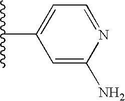 |
| 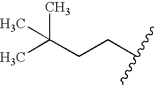 | 5-F | —C(O)— | —CH₂— | 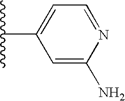 |
| 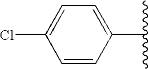 | H | —C(O)— | —CH₂— | 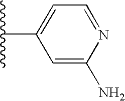 |
| 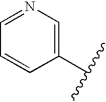 | 5-Cl | —C(O)— | —CH₂— | 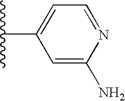 |
| 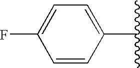 | 5-Cl | —C(O)— | —CH₂— | 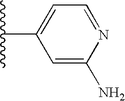 |
| H | 5-CF₃— | —C(O)— | —NH—C(O)— | 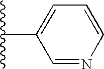 |
| CH₃O(CH₂)₂— | 5-CF₃— | —C(O)— | bond | 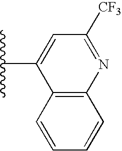 |
| CH₃O(CH₂)₂— | H | —C(O)— | —CH₂— | 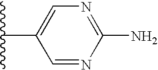 |
| CH₃O(CH₂)₂— | H | —C(O)— | —CH₂— | 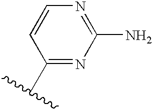 |
| CH₃O(CH₂)₂— | H | —C(O)— | —CH₂— | 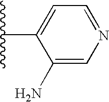 |
| 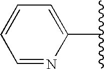 | 5-F | —C(O)— | —CH₂— | 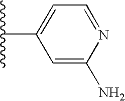 |
| 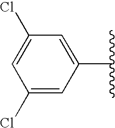 | 5-F | —C(O)— | —CH₂— | 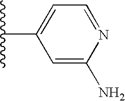 |
| 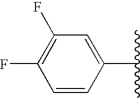 | 5-F | —C(O)— | —CH₂— | 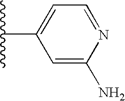 |
| 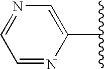 | 5-F | —C(O)— | —CH₂— | 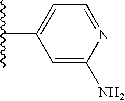 |
| 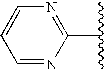 | 5-F | —C(O)— | —CH₂— | 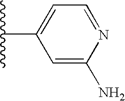 |
| 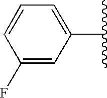 | 5-F | —C(O)— | —CH₂— | 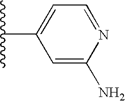 |
| 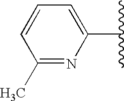 | 5-F | —C(O)— | —CH₂— | 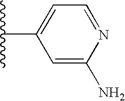 |
| 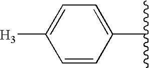 | 5-F | —C(O)— | —CH₂— | 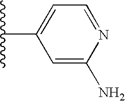 |
| 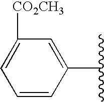 | 5-F | —C(O)— | —CH₂— | 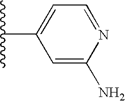 |
| H | 5-F | —C(O)— | —CH₂— | 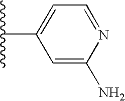 |
| CF₃CH₂— | 5-F | —C(O)— | —CH₂— | 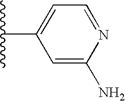 |
| 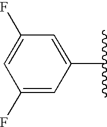 | 5-F | —C(O)— | —CH₂— | 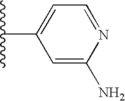 |
| 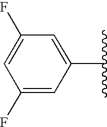 | 5-Cl | —C(O)— | —CH₂— | 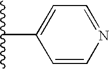 |
| 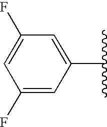 | 5-Cl | —C(O)— | —CH₂— | 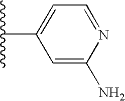 |
| 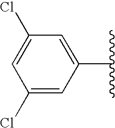 | 5-Cl | —C(O)— | —CH₂— | 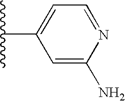 |
| 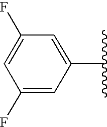 | 5-Cl | —C(O)— | —CH(CH₃)— | 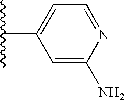 |
| 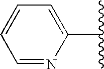 | 5-CF₃— | —C(O)— | —CH₂— | 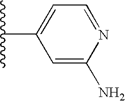 |
| 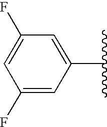 | 5-CF₃— | —C(O)— | —CH₂— | 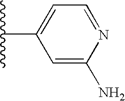 |
| 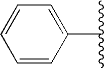 | 5-CF₃— | —C(O)— | —CH₂— | 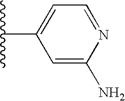 |
| H | 5-CF₃— | —C(O)— | —CH₂— | 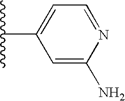 |

compounds of the following general formula:

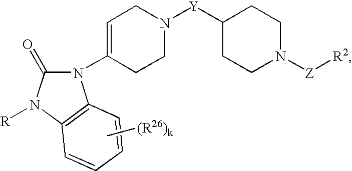

wherein R, (R²⁶)ₖ, Y, Z, and R² are as defined in the following Table:

| R | (R²⁶)ₖ | Y | Z | R² |
|---|---|---|---|---|
| H | H | —C(O)— | —CH₂— | 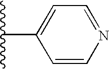 |
| N(CH₃)₂—(CH₂)₂— | H | —C(O)— | —CH₂— | 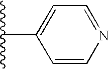 |
| H | H | —C(O)— | —C(O)— | 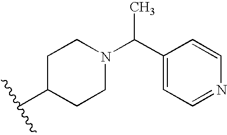 |

compounds of the following general formula:

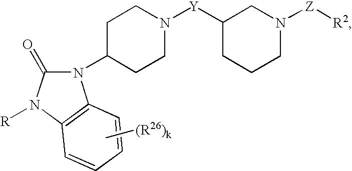

wherein R, (R²⁶)ₖ, Y, Z, and R² are as defined in the following Table:

| R | (R²⁶)ₖ | Y | Z | R² |
|---|---|---|---|---|
| H | 5-CF₃— | —C(O)— | —CH₂— | 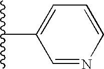 |
| H | 5-CF₃— | —C(O)— | —CH₂— | 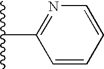 |
| H | 5-CF₃— | —C(O)— | —C(O)— | 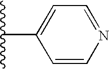 |
| H | 5-CF₃— | —C(O)— | —CH₂— | 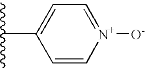 |

compounds of the following general formula:

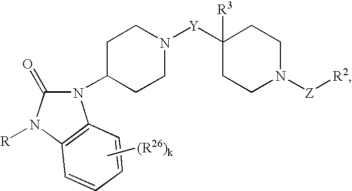

wherein R, (R²⁶)ₖ, Y, Z, R³, and R² are as defined in the following Table:

| R | (R²⁶)ₖ | Y | R³ | Z | R² |
|---|---|---|---|---|---|
| H | H | —C(O)— | —CH₃ | —CH(CH₃)— | 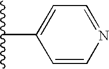 |
| H | 5-CF₃— | —C(O)— | —CH₃ | —CH₂— | 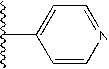 |
| H | 5-CF₃— | —C(O)— | —OH | —CH₂— | 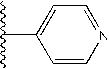 |
| CH₃O(CH₂)₂— | H | —C(O)— | F | —CH(CH₃)— | 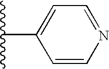 |
| H | H | —C(O)— | —CH₃ | —CH₂— | 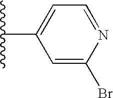 |
| CH₃O(CH₂)₂— | 5-Cl | —C(O)— | F | —CH(CH₃)— | 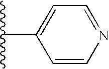 |
| CH₃C(O)(CH₂)₂— | 5-CF₃— | —C(O)— | —CH₃ | —CH₂— | 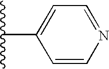 |
| CH₃O(CH₂)₂— | H | —C(O)— | F | —CH₂— | 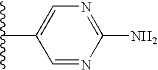 |
| CH₃O(CH₂)₂— | H | —C(O)— | F | —CH₂— | 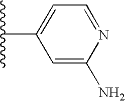 |
| H | 5-F | —C(O)— | F | —CH₂— | 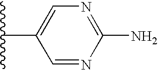 |

compounds of the following general formula:

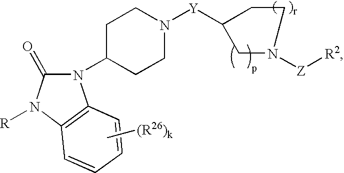

wherein R, (R²⁶)ₖ, Y, r, p, Z, and R² are defined as in the following Table:

| R | (R²⁶)ₖ | Y | r | p | Z | R² |
|---|---|---|---|---|---|---|
| CH₃O(CH₂)₂— | H | —C(O)— | 0 | 1 | —CH₂— | 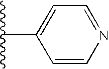 |
| CH₃O(CH₂)₂— | H | —C(O)— | 1 | 1 | —CH₂— | 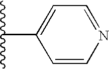 |
| CH₃O(CH₂)₂— | H | —C(O)— | 1 | 3 | —CH₂— | 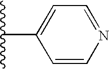 |

compounds of the following general formula:

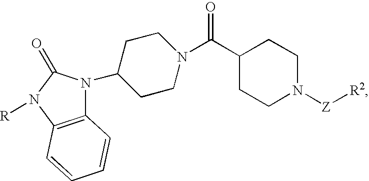

wherein R, Z, and R² are defined as in the following Table:

| R | Z | R² |
|---|---|---|
| CH₃O(CH₂)₂— | —C(O)— | 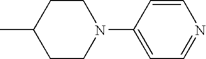 |
| CH₃O(CH₂)₂— | —C(O)— | 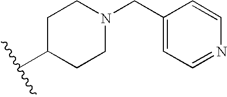 |
| CH₃O(CH₂)₂— | —C(O)— | 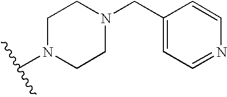 |
| CH₃O(CH₂)₂— | —C(O)— | 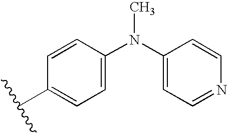 |
| CH₃O(CH₂)₂— | 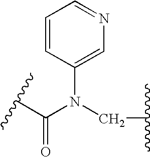 | 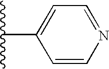 |
| CH₃O(CH₂)₂— | —C(O)—NH— | 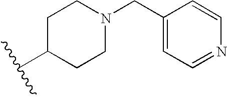 |
| 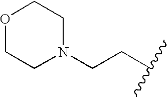 | —C(O)— | 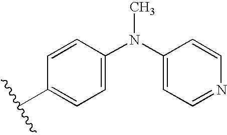 |
| CH₃O(CH₂)₂— | —C(O)— | 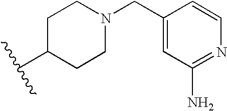 |
| H | —CH₂— | 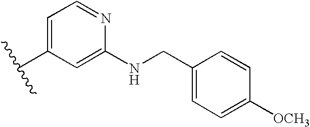 |

compounds of the following general formula:

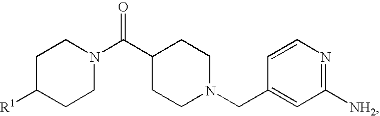

wherein R¹ is defined as shown in the following Table:

R¹

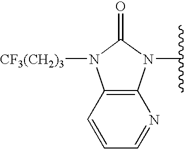

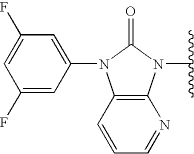

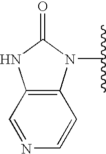

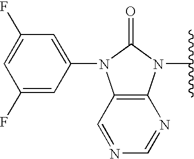

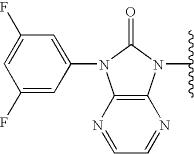

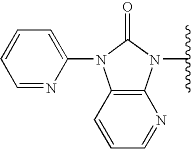

compounds of the following general formula:

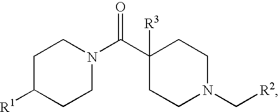

wherein R¹, R³, and R² are defined as shown in the following Table:

| R¹ | R³ | R² |
|---|---|---|
| 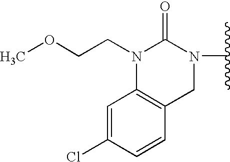 | F | 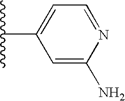 |
| 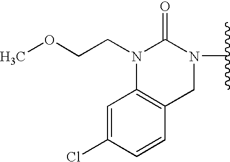 | H | 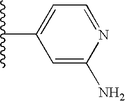 |
| 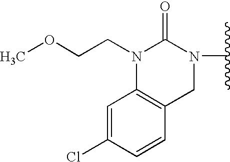 | H | 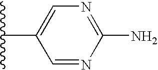 |
| 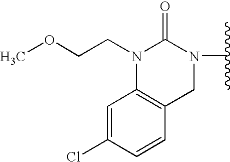 | F | 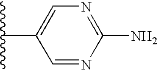 |
| 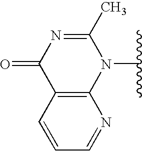 | H | 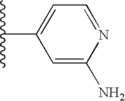 |
| 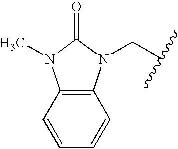 | H | 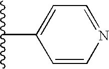 |

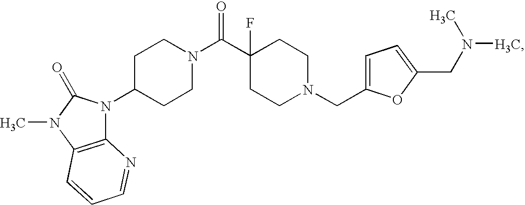

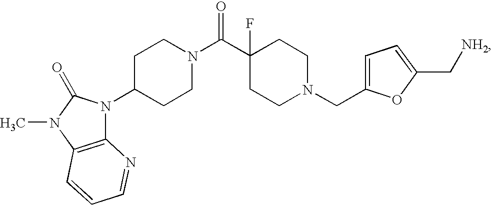

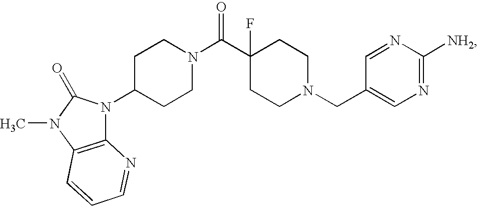

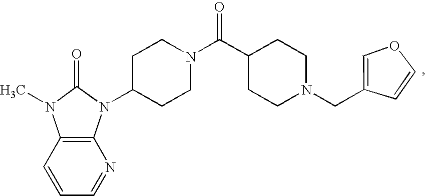

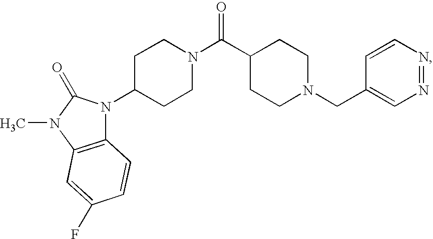

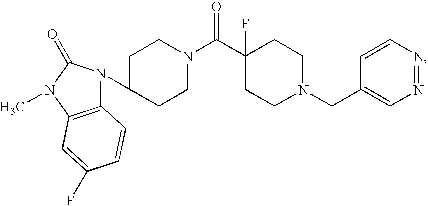

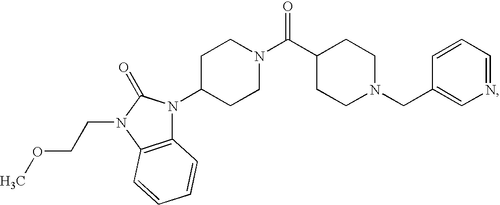

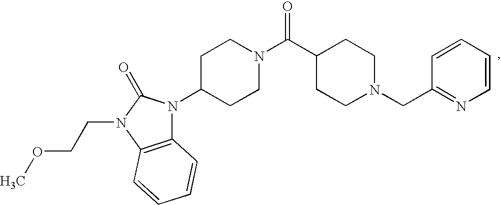

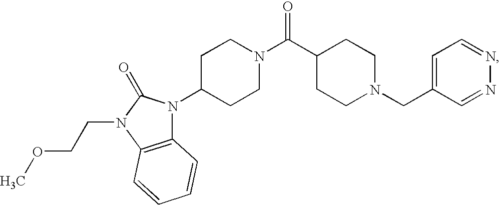

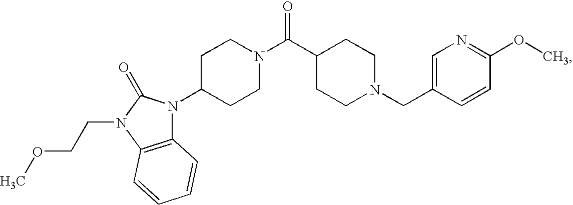

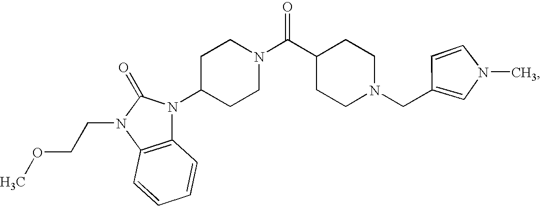

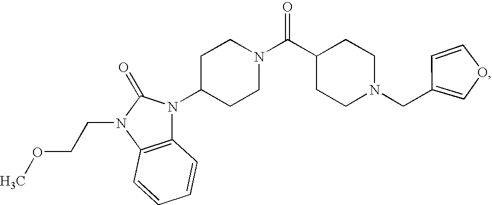

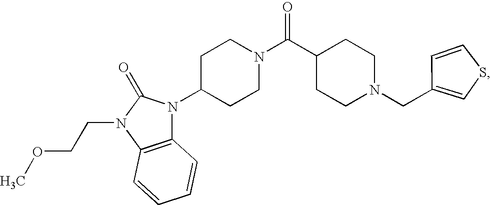

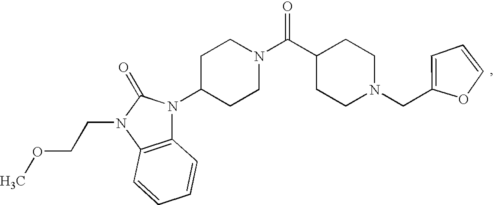

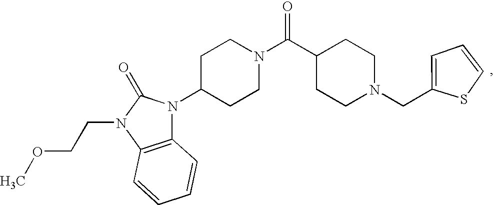

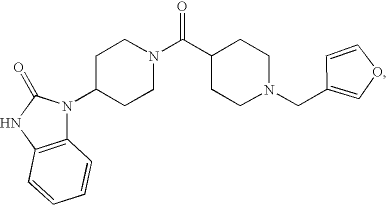

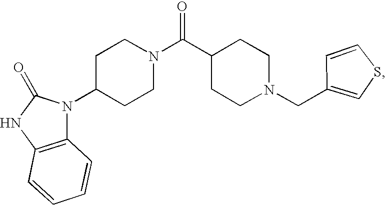

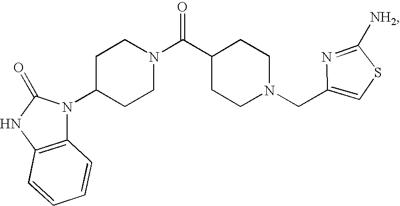

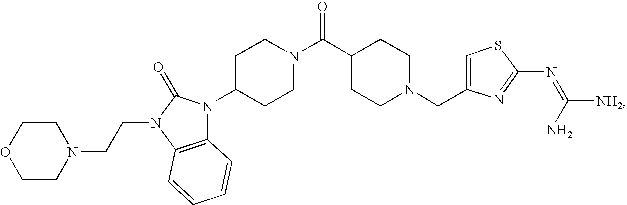

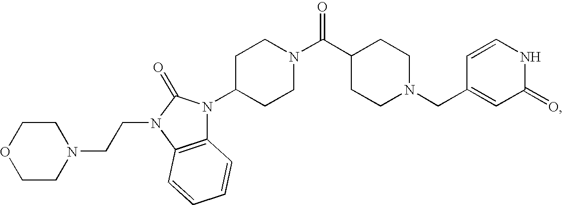

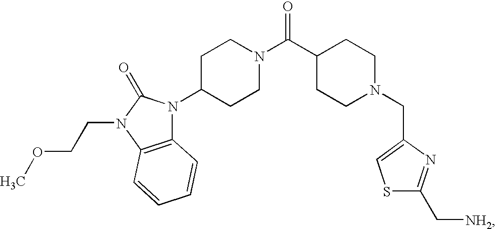

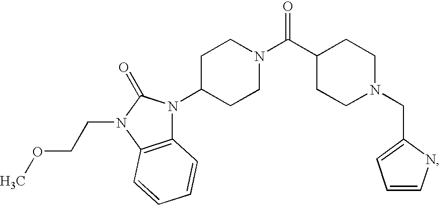

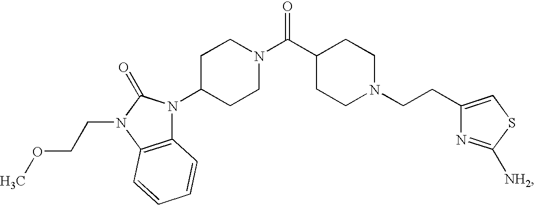

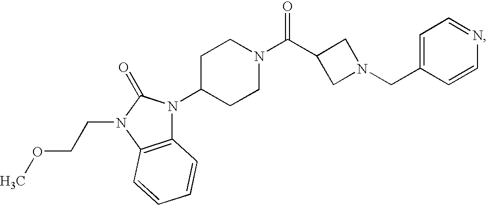

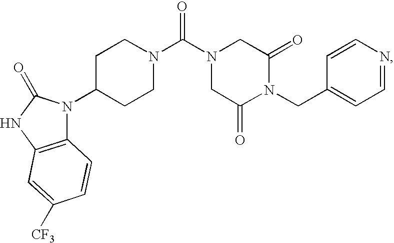

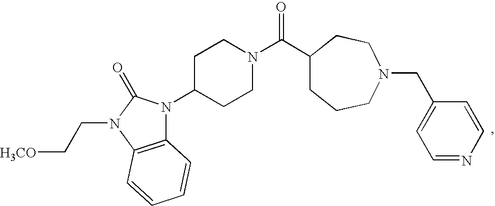

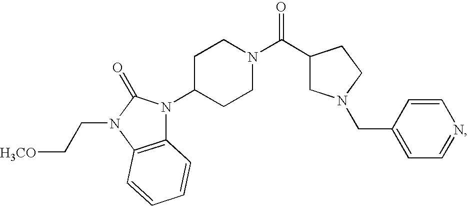

compounds of the following general formula:

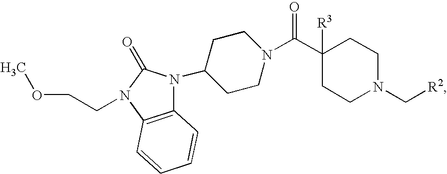

wherein R³ and R² are defined as shown in the following Table:

| R³ | R² |
|---|---|
| H | 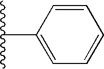 |
| F | 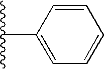 |
| F | 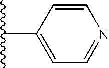 |
| F | 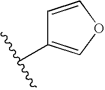 |
| F | 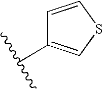 |
| F | 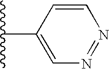 |
| F | 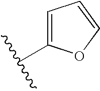 |
| F | 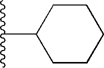 |
| F | 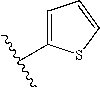 |
| F | 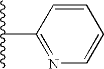 |

compounds of the following general formula:

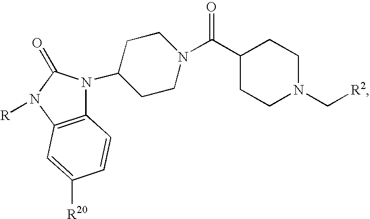

wherein R, R²⁰, and R² are defined as shown in the following Table:

| R | R²⁰ | R² |
|---|---|---|
| 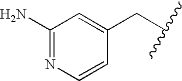 | H | 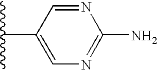 |
| 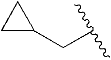 | F | 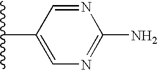 |
| 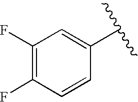 | F | 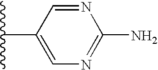 |
| 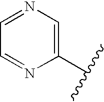 | F | 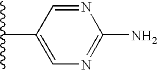 |
| 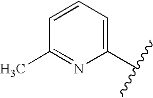 | F | 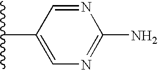 |
| 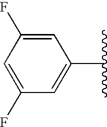 | F | 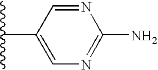 |
| 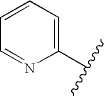 | —CF₃ | 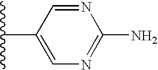 |
| CF₃(CH₂)₃— | F | 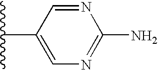 |
| 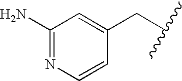 | H | 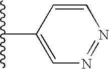 |
| 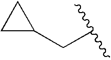 | F | 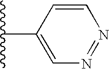 |
| 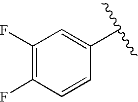 | F | 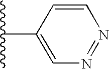 |
| 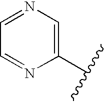 | F | 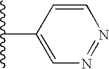 |
| 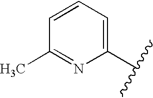 | F | 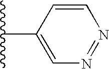 |
| 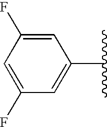 | F | 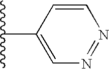 |
| 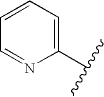 | —CF₃ | 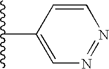 |
| CF₃(CH₂)₃— | F | 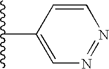 |

In one embodiment, the H₃ antagonists/inverse agonists of the present invention can have a structure according to Formula (V): 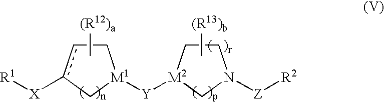
as described in U.S. 2004/0019099, which is herein incorporated by reference in its entirety. Non-limiting examples of compounds of Formula (V) include: 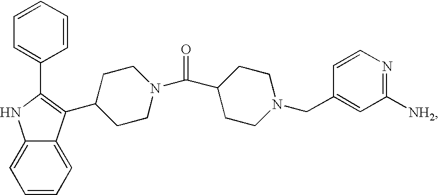
compounds of the following general formula:

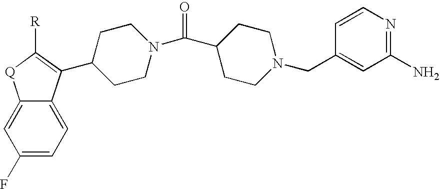

wherein Q and R are as defined in the following Table:

| Q | R |
|---|---|
| O | 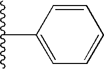 |
| O | —CH₃ |
| S | —CH₃ |
| S | —C(O)—O—CH₂CH₃ |
| O | H |

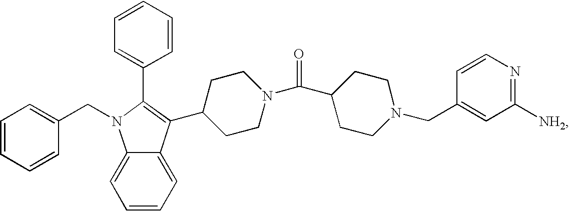

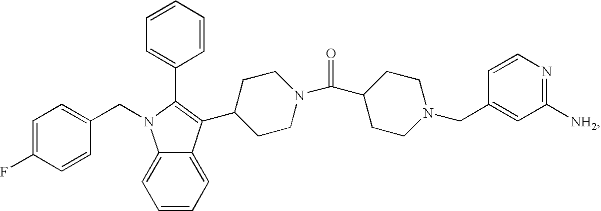

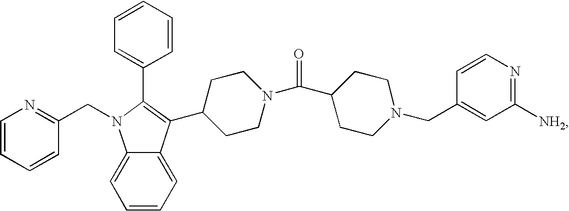

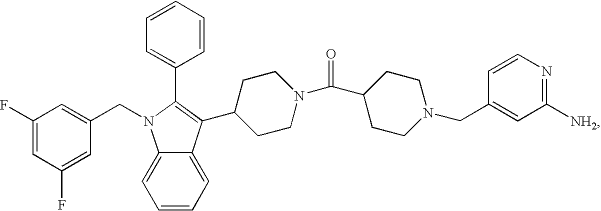

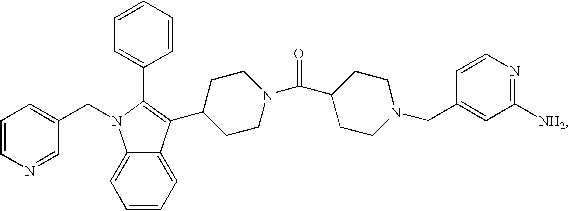

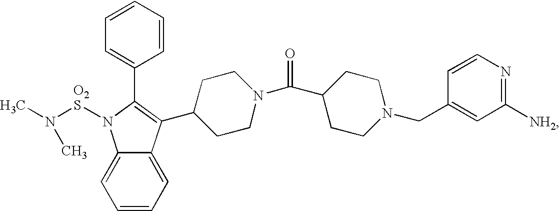

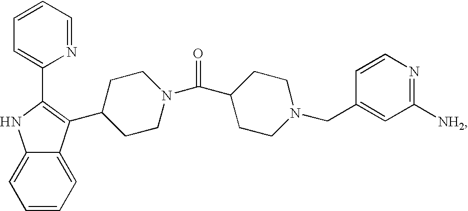

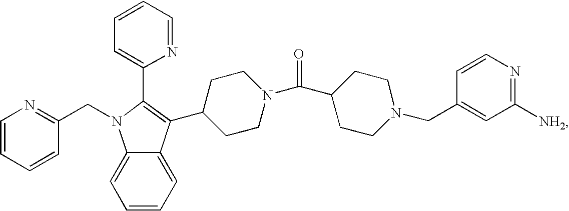

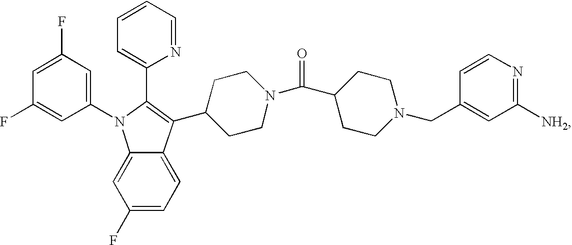

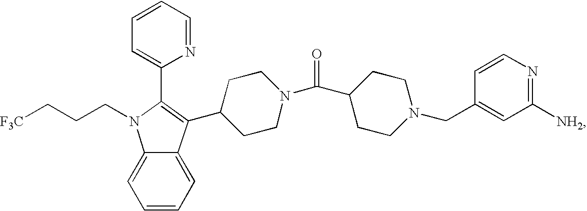

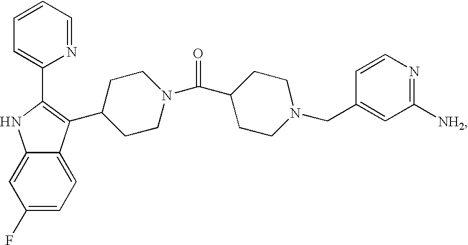

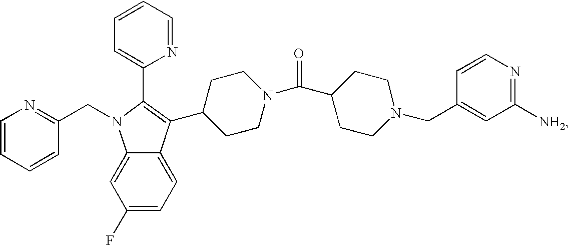

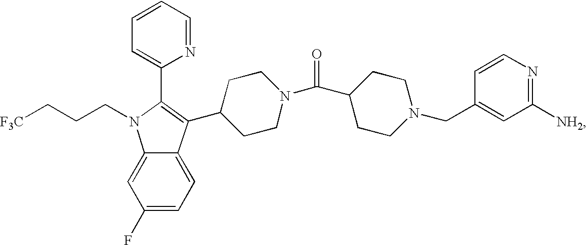

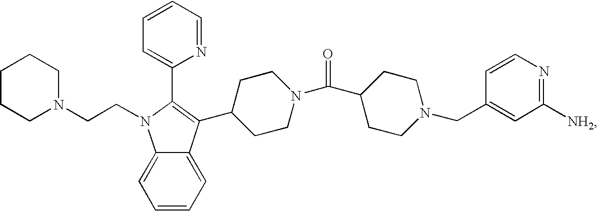

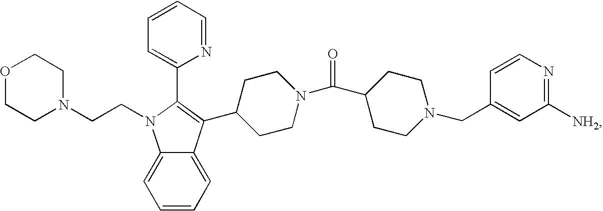

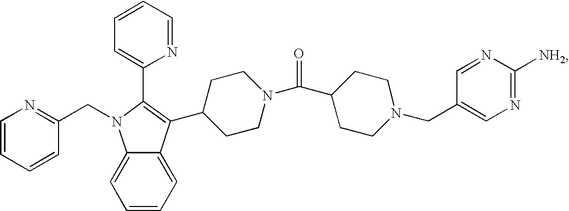

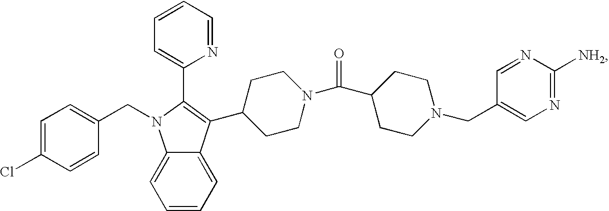

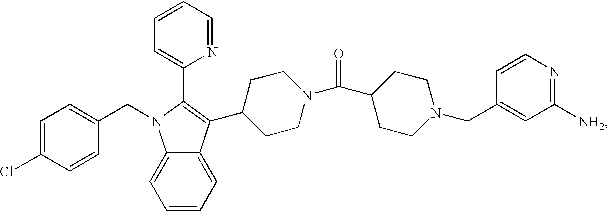

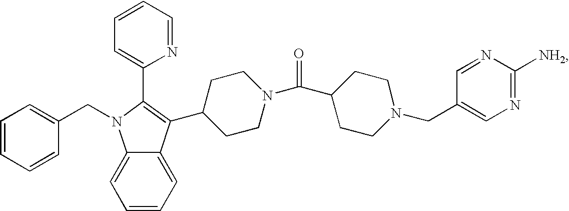

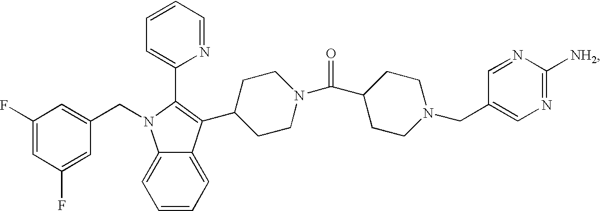

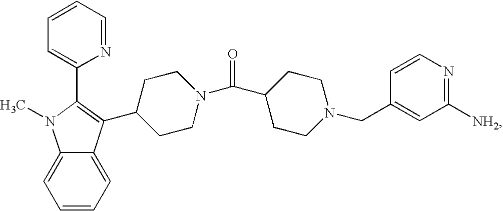

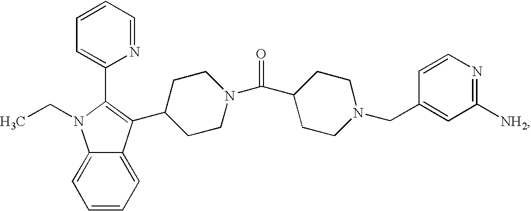

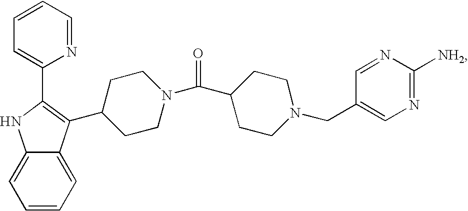

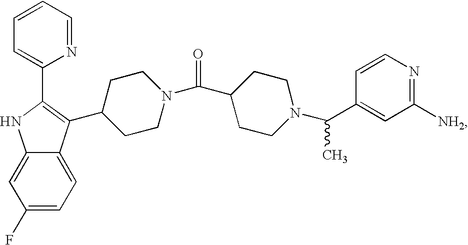

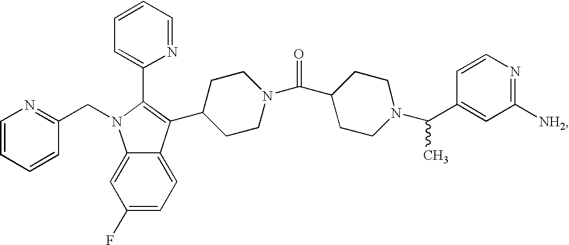

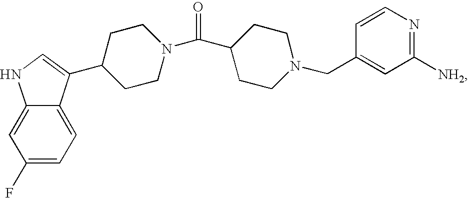

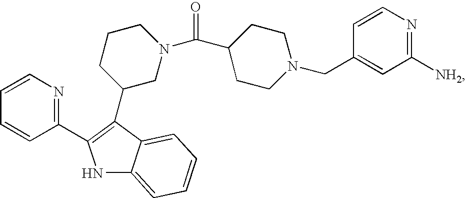

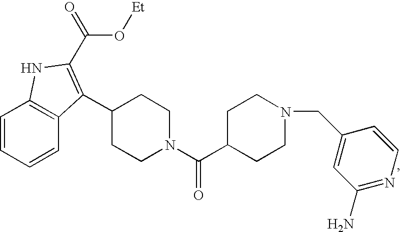

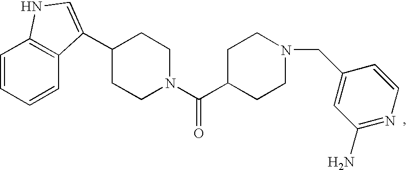

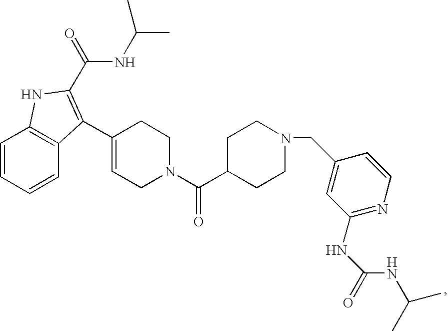

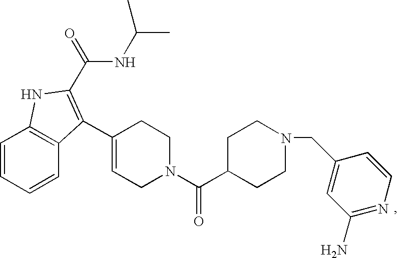

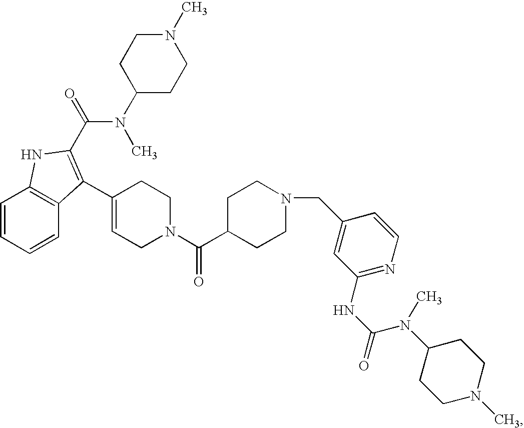

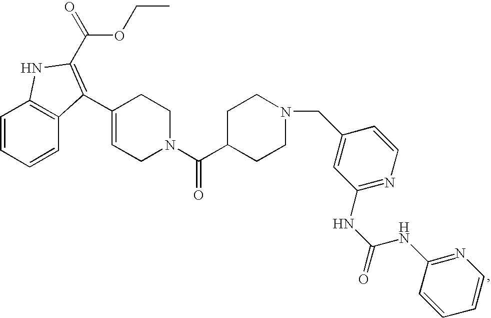

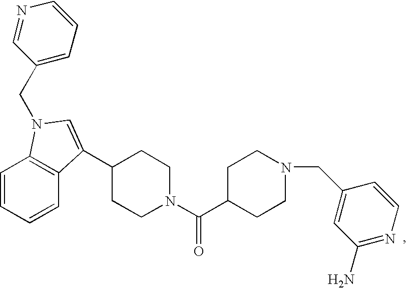

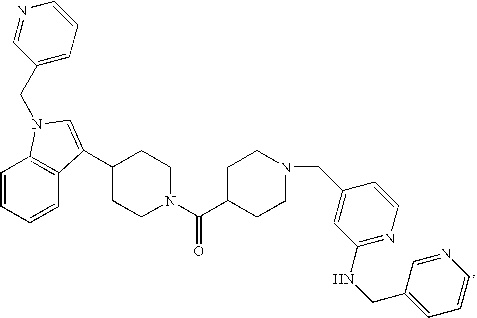

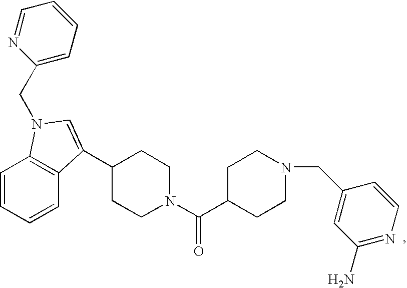

compounds of the following general formula:

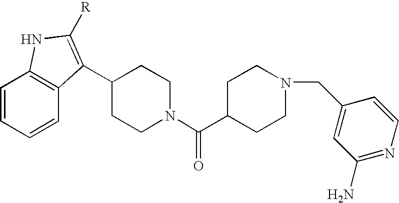

wherein R is as defined in the following Table:

R

—C(O)—NH—CH₃
—C(O)—NH—CH₂CH₃

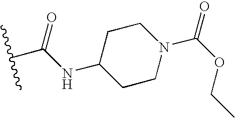

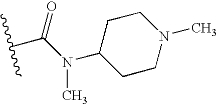

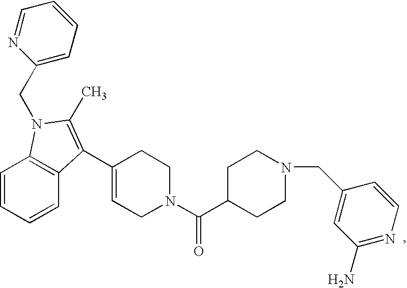

compounds of the following general formula:

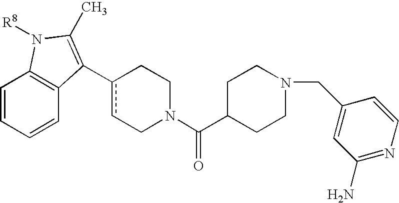

wherein R is as defined in the following Table:

| | Optional |
|---|---|
| R⁸ | Double Bond |
| 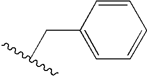 | present |
| CF₃—(CH₂)₃— | present |
| CH₃—CH₂— | present |
| 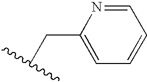 | absent |
| H | absent |
| 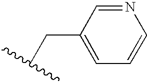 | absent |

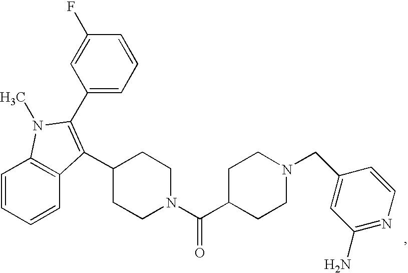

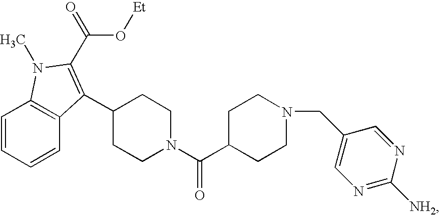

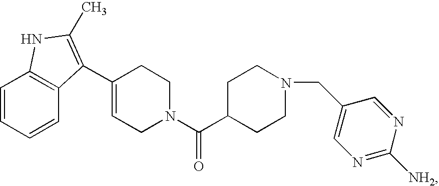

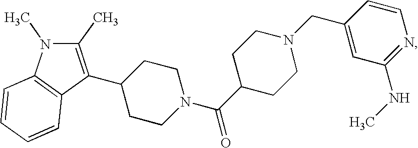

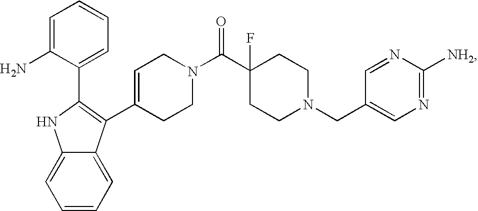

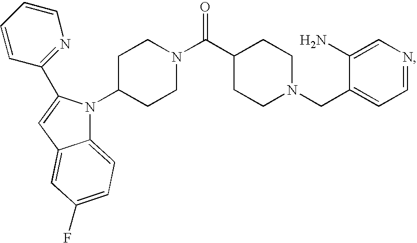

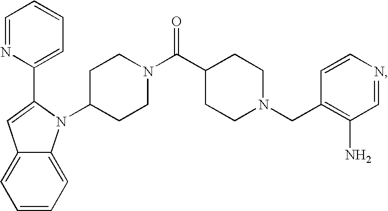

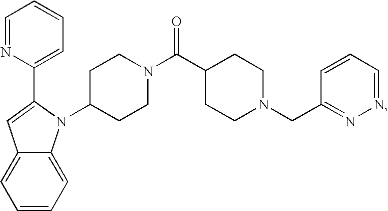

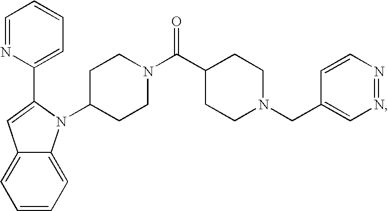

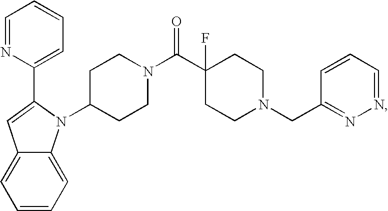

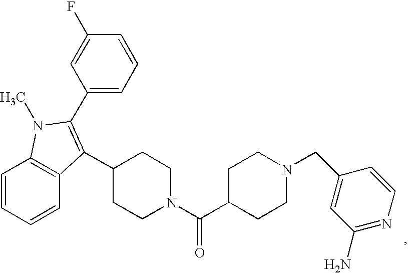

compounds of the following general formula:

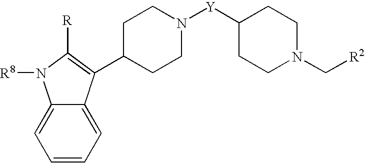

wherein R, R⁸ and R² are as defined in the following Table:

| R | R⁸ | Y | R² |
|---|---|---|---|
| 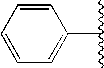 | 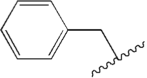 | —C(O)— | 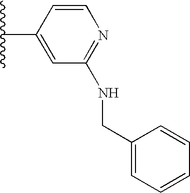 |
| 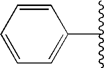 | 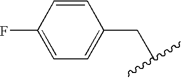 | —C(O)— | 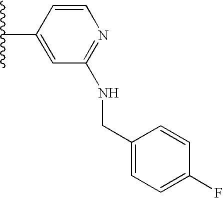 |
| 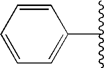 | 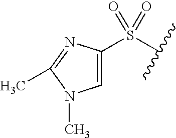 | —C(O)— | 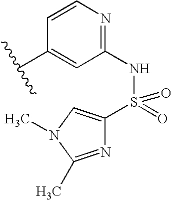 |
| 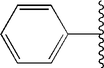 | H | bond | 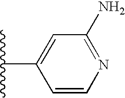 |
| 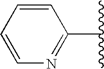 | CH₃CH₂— | —C(O)— | 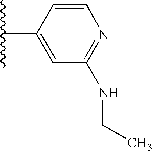 |
| 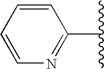 | 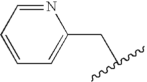 | —C(O)— | 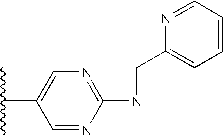 |
| 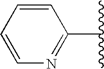 | 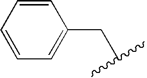 | —C(O)— | 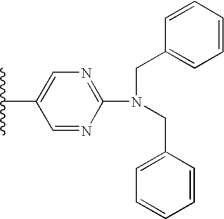 |
| 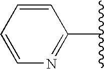 | 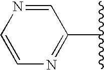 | —C(O)— | 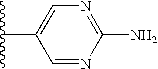 |
| 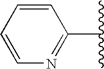 | 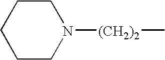 | —C(O)— | 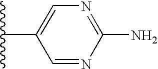 |
| 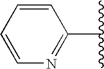 | H | —C(O)— | 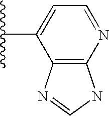 |

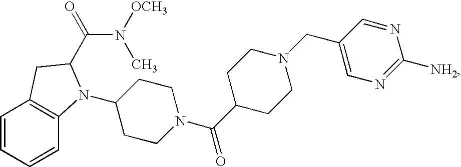

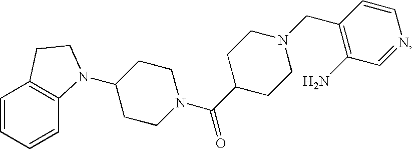

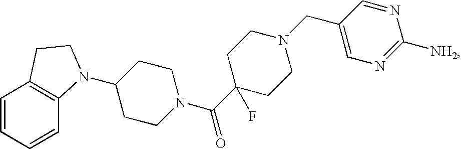

and

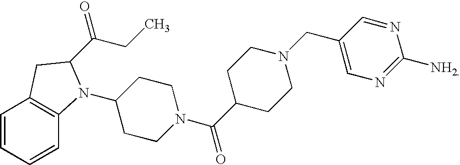

In one embodiment, the H₃ antagonists/inverse agonists of the present invention can have a structure according to Formula (VI): 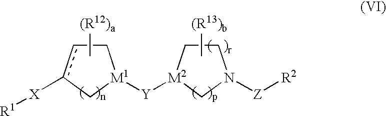
as described in U.S. 2004/0097483, which is herein incorporated by reference in its entirety. Non-limiting examples of compounds of Formula (VI) include: 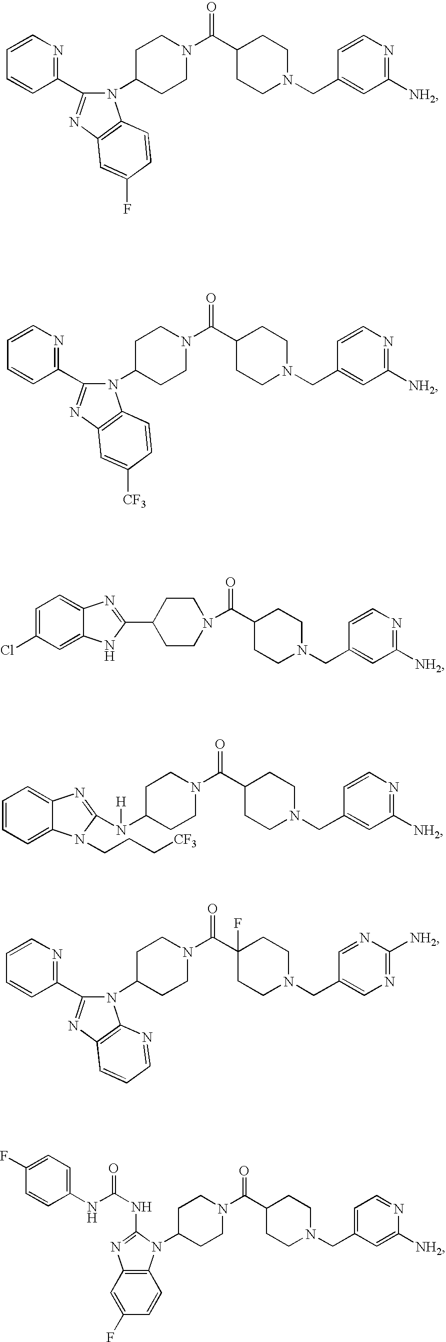
compounds of the following formula:

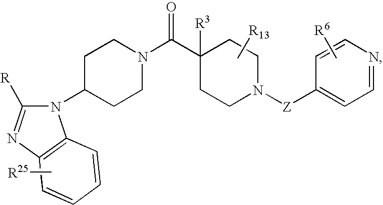

wherein R, R²⁵, R³, R¹³, Z and R⁶ are as shown in the following table:

| | | | | | | | Physical |
|---|---|---|---|---|---|---|---|
| | | | | | | | Data |
| No. | R | R²⁵ | R³ | R¹³ | Z | R⁶ | MS (MH⁺) |
| 7 | —CH₃ | 5-OCH₃ | H | H | —CH₂— | 2-NH₂ | 463 |
| 8 | —CH₃ | 6-Cl | H | H | —CH₂— | 2-NH₂ | 467 |
| 9 | —CH₃ | 5-Cl | H | H | —CH₂— | 2-NH₂ | 467 |
| 10 | —CH₃ | 5-Br | H | H | —CH₂— | 2-NH₂ | 512 |
| 11 | 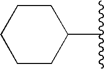 | 5-Cl | H | H | —CH₂— | 2-NH₂ | 535 |
| 12 | benzyl | 5-F | H | H | —CH₂— | 2-NH₂ | 527 |
| 13 | —CH(CH₃)₂ | 5-Br | H | H | —CH₂— | 2-NH₂ | 540 |
| 14 | —CH₂NH₂ | H | H | H | —CH₂— | 2-NH₂ | 488 |
| 15 | —CH₂NHSO₂CH₃ | H | H | H | —CH₂— | 2-NH₂ | 526 |
| 16 | —CH₂NHC(O)CH₃ | 5-Cl | H | H | —CH₂— | 2-NH₂ | 524 |
| 17 | —CH₂OCH₃ | 5-F | H | H | —CH₂— | 2-NH₂ | 481 |
| 18 | —CH₂NH₂ | 5-Cl | H | H | —CH₂— | 2-NH₂ | 482 |
| 19 | —CH₂OCH₃ | 6,7-di-F | H | H | —CH₂— | 2-NH₂ | 499 |
| 20 | 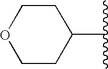 | 6-F | H | H | —CH₂— | 2-NH₂ | 521 |
| 21 | 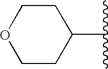 | 5-F | H | H | —CH₂— | 2-NH₂ | 521 |
| 22 | 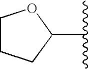 | 6-F | H | H | —CH₂— | 2-NH₂ | 507 |
| 23 | 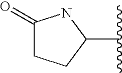 | 5-F | H | H | —CH₂— | 2-NH₂ | 520 |
| 24 | 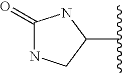 | 5-F | H | H | —CH₂— | 2-NH₂ | 521 |
| 25 | 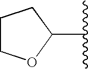 | 5-Br | H | H | —CH₂— | 2-NH₂ | 568 |
| 26 | 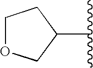 | 5-F | H | H | —CH₂— | 2-NH₂ | 507 |
| 27 | 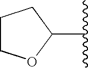 | 5-F | H | H | —CH₂— | 2-NH₂ | 507 |
| 28 | 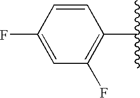 | H | H | H | —CH₂— | 2-NH₂ | 531 |
| 29 | 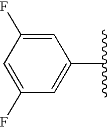 | 5-F | H | H | —CH₂— | 2-NH₂ | 549 |
| 30 | 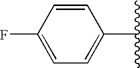 | 6-F | H | H | —CH₂— | 2-NH₂ | 531 |
| 31 | 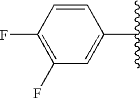 | 6,7-di-F | H | H | —CH₂— | 2-NH₂ | 567 |
| 32 | 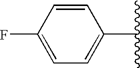 | 6-Cl | H | H | —CH₂— | 2-NH₂ | 547 |
| 33 | 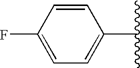 | 5-F | H | H | —CH₂— | 2-NH₂ | 531 |
| 34 | 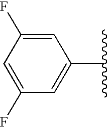 | 5-Cl | H | H | —CH₂— | 2-NH₂ | 565 |
| 35 | 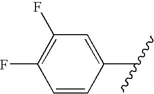 | H | H | H | —CH₂— | 2-NH₂ | 531 |
| 36 | 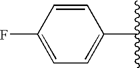 | 5-Cl | H | H | —CH₂— | 2-NH₂ | 547 |
| 37 | 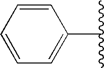 | 5-Cl | H | H | —CH₂— | 2-NH₂ | 529 |
| 38 | 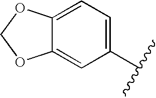 | 6-F | H | H | —CH₂— | 2-NH₂ | 557 |
| 39 | 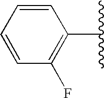 | 5-Br | H | H | —CH₂— | 2-NH₂ | 592 |
| 40 | 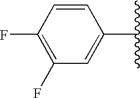 | 5-Br | H | H | —CH₂— | 2-NH₂ | 610 |
| 41 | 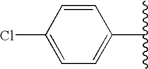 | 5-F | H | H | —CH₂— | 2-NH₂ | 547 |
| 42 | 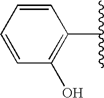 | 5-F | H | H | —CH₂— | 2-NH₂ | 529 |
| 43 | 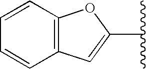 | 6-F | H | H | —CH₂— | 2-NH₂ | 553 |
| 44 | 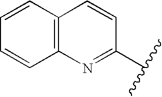 | 6-F | H | H | —CH₂— | 2-NH₂ | 564 |
| 45 | 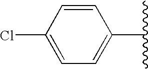 | H | H | H | —CH₂— | 2-NH₂ | 529 |
| 46 | 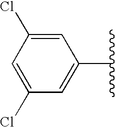 | 5-F | H | H | —CH₂— | 2-NH₂ | 581 |
| 47 | 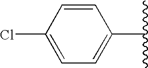 | 5-Cl | H | H | —CH₂— | 2-NH₂ | 563 |
| 48 | 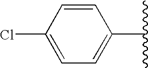 | 6-Cl | H | H | —CH₂— | 2-NH₂ | 563 |
| 49 | 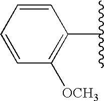 | 5-F | H | H | —CH₂— | 2-NH₂ | 543 |
| 50 | 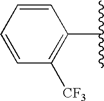 | 5-F | H | H | —CH₂— | 2-NH₂ | 581 |
| 51 | 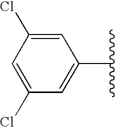 | 5-Cl | H | H | —CH₂— | 2-NH₂ | 597 |
| 52 | 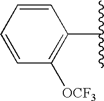 | 5-F | H | H | —CH₂— | 2-NH₂ | 597 |
| 53 | 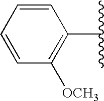 | 5-Br | H | H | —CH₂— | 2-NH₂ | 604 |
| 54 | 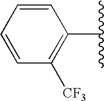 | 6-Cl | H | H | —CH₂— | 2-NH₂ | 597 |
| 55 | 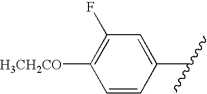 | 5-CH₃ | H | H | —CH₂— | 2-NH₂ | 571 |
| 56 | 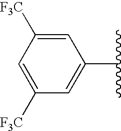 | 5-Cl | H | H | —CH₂— | 2-NH₂ | 665 |
| 57 | 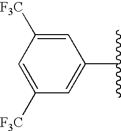 | 5-Br | H | H | —CH₂— | 2-NH₂ | 710 |
| 58 | 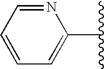 | 6-ethoxy | H | H | —CH₂— | 2-NH₂ | 540 |
| 59 | 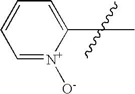 | 5-Cl | H | H | —CH₂— | 2-NH₂ | 546 |
| 60 | 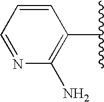 | H | H | H | —CH₂— | 2-NH₂ | 511 |
| 61 | 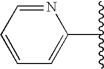 | 5-F | H | H | —CH₂— | H | 499 |
| 62 | 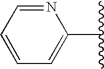 | 6-Cl | H | H | —CH₂— | 2-NH₂ | 530 |
| 63 | 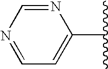 | 5-F | H | H | —CH₂— | 2-NH₂ | 515 |
| 64 | 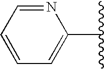 | 6-F | H | H | —CH₂— | 2-NH₂ | 514 |
| 65 | 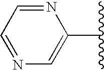 | 6-F | H | H | —CH₂— | 2-NH₂ | 515 |
| 66 | 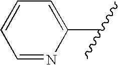 | 7-Cl | H | H | —CH₂— | 2-NH₂ | 531 |
| 67 | 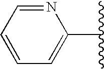 | H | H | H | —CH₂— | 2-NH₂ | 496 |
| 68 | 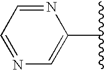 | 5-F | H | H | —CH₂— | 2-NH₂ | 515 |
| 69 | 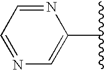 | 5-Cl | H | H | —CH₂— | 2-NH₂ | 531 |
| 70 | 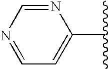 | 5-Cl | H | H | —CH₂— | 2-NH₂ | 531 |
| 71 | 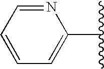 | 5,6-di-F | H | H | —CH₂— | 2-NH₂ | 532 |
| 72 | 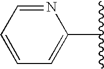 | 5-Br | H | H | —CH₂— | 2-NH₂ | 575 |
| 73 | 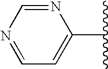 | 6-ethoxy | H | H | —CH₂— | 2-NH₂ | 541 |
| 74 | 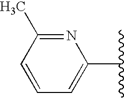 | 5-F | H | H | —CH₂— | 2-NH₂ | 528 |
| 75 | 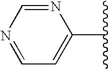 | 6-F | H | H | —CH₂— | 2-NH₂ | 515 |
| 76 | 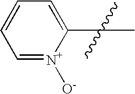 | 5-Br | H | H | —CH₂— | 2-NH₂ | 591 |
| 77 | 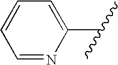 | 5-Cl | H | H | —CH₂— | 2-NH₂ | 530 |
| 78 | 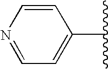 | 5-Cl | H | H | —CH₂— | 2-NH₂ | 530 |
| 79 | 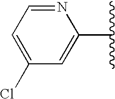 | 5-F | H | H | —CH₂— | 2-NH₂ | 548 |
| 80 | 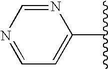 | 5-CF₃ | H | H | —CH₂— | 2-NH₂ | 565 |
| 81 | 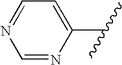 | H | H | H | —CH₂— | 2-NH₂ | 497 |
| 82 | 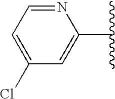 | 6,7-di-F | H | H | —CH₂— | 2-NH₂ | 567 |
| 83 | 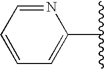 | 6,7-di-F | H | H | —CH₂— | 2-NH₂ | 532 |
| 84 | 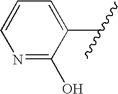 | 5-F | H | H | —CH₂— | 2-NH₂ | 530 |
| 85 | 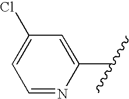 | 5-CF₃,7-F | H | H | —CH₂— | 2-NH₂ | 617 |
| 86 | 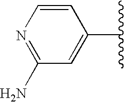 | 5-F | H | H | —CH₂— | 2-NH₂ | 529 |
| 87 | 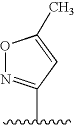 | H | H | H | —CH₂— | 2-NH₂ | 500 |
| 88 | 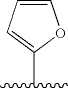 | H | H | H | —CH₂— | 2-NH₂ | 485 |
| 89 | 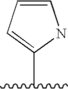 | H | H | H | —CH₂— | 2-NH₂ | 489 |
| 90 | 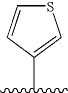 | 6-F | H | H | —CH₂— | 2-NH₂ | 514 |
| 91 | 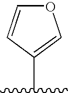 | 6-F | H | H | —CH₂— | 2-NH₂ | 503 |
| 92 | 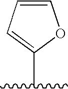 | 5-F | H | H | —CH₂— | 2-NH₂ | 503 |
| 93 | 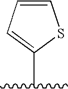 | H | H | H | —CH₂— | 2-NH₂ | 501 |
| 94 | 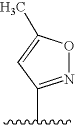 | 5-F | H | H | —CH₂— | 2-NH₂ | 518 |
| 95 | 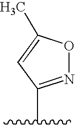 | 5-Cl | H | H | —CH₂— | 2-NH₂ | 534 |
| 96 | 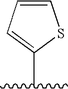 | 5-F | H | H | —CH₂— | 2-NH₂ | 519 |
| 97 | 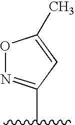 | 6,7-di-F | H | H | —CH₂— | 2-NH₂ | 536 |
| 98 | 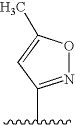 | 5-Br | H | H | —CH₂— | 2-NH₂ | 579 |
| 99 | 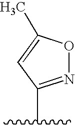 | 6-ethoxy | H | H | —CH₂— | 2-NH₂ | 544 |
| 100 | 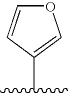 | 5-F | H | H | —CH₂— | 2-NH₂ | 503 |
| 101 | 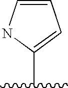 | 5-Br | H | H | —CH₂— | 2-NH₂ | 563 |
| 102 | 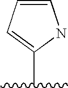 | 5-F | H | H | —CH₂— | 2-NH₂ | 502 |
| 103 | 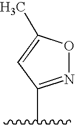 | 5-CF₃ | H | H | —CH₂— | 2-NH₂ | 568 |
| 104 | 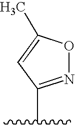 | 5-CF₃,7-F | H | H | —CH₂— | 2-NH₂ | 586 |
| 105 | 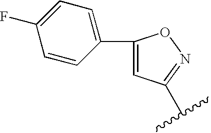 | 5-F | H | H | —CH₂— | 2-NH₂ | 598 |
| 106 | 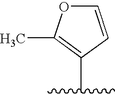 | 5-F | H | H | —CH₂— | 2-NH₂ | 517 |
| 107 | 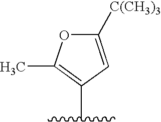 | 5-F | H | H | —CH₂— | 2-NH₂ | 573 |
| 108 | 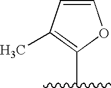 | 5-F | H | H | —CH₂— | 2-NH₂ | 517 |
| 109 | CH₃—S— | 5-F | H | H | —CH₂— | 2-NH₂ | 483 |
| 110 | CH₃—CH₂—S— | 5-F | H | H | —CH₂— | 2-NH₂ | 497 |
| 111 | CH₃—SO₂— | 5-F | H | H | —CH₂— | 2-NH₂ | 515 |
| 112 | 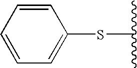 | 5-F | H | H | —CH₂— | 2-NH₂ | 545 |
| 113 | 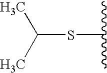 | 5-F | H | H | —CH₂— | 2-NH₂ | 511 |
| 114 | 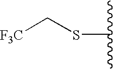 | 5-F | H | H | —CH₂— | 2-NH₂ | 551 |
| 115 | 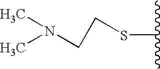 | 5-F | H | H | —CH₂— | 2-NH₂ | 540 |
| 116 | HS— | 5-F | H | H | —CH₂— | 2-NH₂ | 469 |
| 117 | CH₃—S— | 5-F | H | 2-CH₃ | —CH₂— | 2-NH₂ | 497 |
| 118 | CH₃—S— | 5-F | F | H | —CH₂— | 2-NH₂ | 501 |
| 119 | 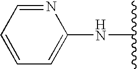 | 5-F | H | H | —CH₂— | 2-NH₂ | 529 |
| 120 | 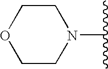 | 5-F | H | H | —CH₂— | 2-NH₂ | 522 |
| 121 | 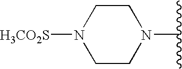 | 5-F | H | H | —CH₂— | 2-NH₂ | 599 |
| 123 | 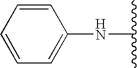 | 5-F | H | H | —CH₂— | 2-NH₂ | 528 |
| 124 | 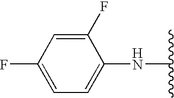 | 5-F | H | H | —CH₂— | 2-NH₂ | 564 |
| 125 | 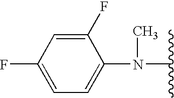 | 5-F | H | H | —CH₂— | 2-NH₂ | 578 |
| 126 | 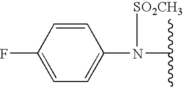 | 5-F | H | H | —CH₂— | 2-NH₂ | 624 |
| 127 | 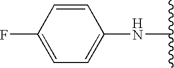 | 5-F | H | H | —CH₂— | 2-NH₂ | 546 |
| 128 | 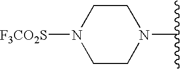 | 5-F | H | H | —CH₂— | 2-NH₂ | 653 |
| 129 | CH₃—O—(CH₂)₂—NH— | 5-F | H | H | —CH₂— | 2-NH₂ | 510 |
| 130 | 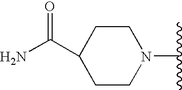 | 5-F | H | H | —CH₂— | 2-NH₂ | 563 |
| 131 | 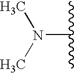 | 5-F | H | H | —CH₂— | 2-NH₂ | 480 |
| 132 | CH₃—O— | 5-F | H | H | —CH₂— | 2-NH₂ | 467 |
| 133 | CH₃—CH₂—O— | 5-F | H | H | —CH₂— | 2-NH₂ | 481 |
| 134 | CH₃—O—(CH₂)₂—O— | 5-F | H | H | —CH₂— | 2-NH₂ | 511 |
| 135 | (CH₃)₂—CH—O— | 5-F | H | H | —CH₂— | 2-NH₂ | 495 |
| 136 | 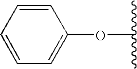 | 5-F | H | H | —CH₂— | 2-NH₂ | 529 |
| 137 | 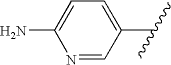 | H | H | H | —CH₂— | 2-NH₂ | 511 |
| 138 | 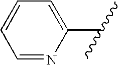 | 5-CF₃,7-F | H | H | —CH₂— | 2-NH₂ | 582 |
| 139 | 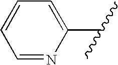 | 5-F | H | H | 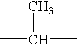 | 2-NH₂ | 528 |
| 140 | 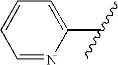 | 5-F | F | H | —CH₂— | 2-NH₂ | 532 |
| 141 | 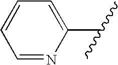 | 5-F | OH | H | —CH₂— | 2-NH₂ | 530 |
| 142 | 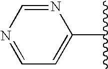 | 5-F | H | H | 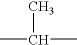 | 2-NH₂ | 529 |
| 143 | 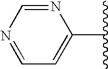 | 5-F | H | H | 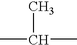 | 2-NH₂ | 529 |
| 144 | 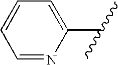 | 5-F | —CH₃ | H | —CH₂— | 2-NH₂ | 528 |
| 145 | 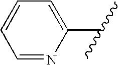 | 6-F | H | H | 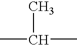 | 2-NH₂ | 528 |
| 146 | H | 5-F | H | H | —CH₂— | 2-NH₂ | 437 |
| 147 | 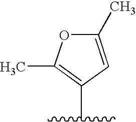 | 5-F | H | H | —CH₂— | 2-NH₂ | 531 |
| 148 | 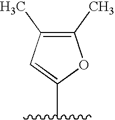 | 5-F | H | H | —CH₂— | 2-NH₂ | 531 |
| 149 | 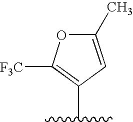 | 5-F | H | H | —CH₂— | 2-NH₂ | 585 |
| 150 | 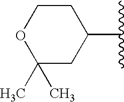 | 5-F | H | H | —CH₂— | 2-NH₂ | 549 |
| 151 | 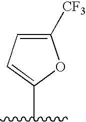 | 5-F | H | H | —CH₂— | 2-NH₂ | 571 |
| 152 | 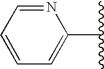 | H | F | H | —CH₂— | 2-NH₂ | 514 |
| 153 | (CH₃)₂N—(CH₂)₂—NH— | 5-F | H | H | —CH₂— | 2-NH₂ | 523 |
| 154 | CH₃—S— | 5-F | H | H | 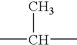 | 2-NH₂ | 497 |
| 155 | 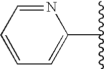 | 5-F | H | 2-CH₃ | —CH₂— | 2-NH₂ | 528 |
| 156 | 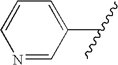 | 5-F | H | H | —CH₂— | 2-NH₂ | 514 |
| 157 | 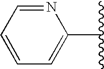 | 5-F | H | H | —CH₂— | 3-NH₂ | 514 |
| 158 | 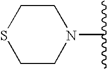 | 5-F | H | H | —CH₂— | 2-NH₂ | 589 |
| 159 | 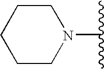 | 5-F | H | H | —CH₂— | 2-NH₂ | 520 |
| 160 | CH₃CH₂O— | 5-F | F | H | —CH₂— | 2-NH₂ | 499 |
| 161 | 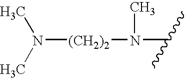 | 5-F | H | H | —CH₂— | 2-NH₂ | 537 |
| 162 | 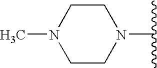 | 5-F | H | H | —CH₂— | 2-NH₂ | 535 |
| 163 | 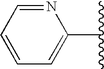 | 5-F | H | 5-OH | —CH₂— | 2-NH₂ | 530 |
| 164 | 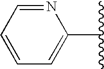 | 5-F | F | H | —CH₂— | 3-NH₂ | 532 |
| 165 | 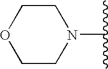 | 5-F | F | H | —CH₂— | 2-NH₂ | 540 |
| 166 | 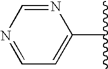 | 5-F | H | H | —CH₂— | 3-NH₂ | 515 |

compounds of the following formula:

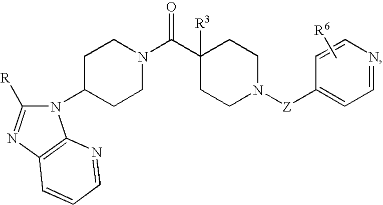

wherein R, R³, Z and R⁶ are as shown in the following table:

| | | | | | Physical |
|---|---|---|---|---|---|
| | | | | | Data |
| No. | R | R³ | Z | R⁶ | MS (MH⁺) |
| 167 | 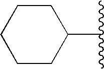 | H | —CH₂— | 2-NH₂ | 502 |
| 168 | —CH₂OCH₃ | H | —CH₂— | 2-NH₂ | 464 |
| 169 | 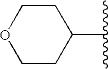 | H | —CH₂— | 2-NH₂ | 504 |
| 170 | 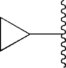 | H | —CH₂— | 2-NH₂ | 460 |
| 171 | (CH₃)₂—CH— | H | —CH₂— | 2-NH₂ | 462 |
| 172 | 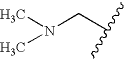 | H | —CH₂— | 2-NH₂ | 477 |
| 173 | 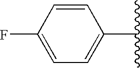 | H | —CH₂— | 2-NH₂ | 514 |
| 174 | 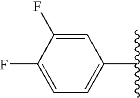 | H | —CH₂— | 2-NH₂ | 532 |
| 175 | 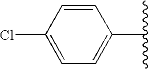 | H | —CH₂— | 2-NH₂ | 530 |
| 176 | 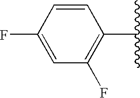 | H | —CH₂— | 2-NH₂ | 532 |
| 177 | 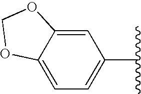 | H | —CH₂— | 2-NH₂ | 540 |
| 178 | 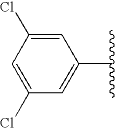 | H | —CH₂— | 2-NH₂ | 564 |
| 179 | 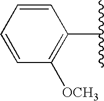 | H | —CH₂— | 2-NH₂ | 526 |
| 180 | 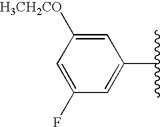 | H | —CH₂— | 2-NH₂ | 558 |
| 181 | 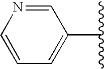 | H | —CH₂— | 2-NH₂ | 497 |
| 182 | 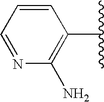 | H | —CH₂— | 2-NH₂ | 512 |
| 183 | 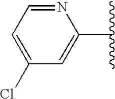 | H | —CH₂— | 2-NH₂ | 531 |
| 184 | 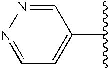 | H | —CH₂— | 2-NH₂ | 498 |
| 185 | 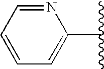 | H | —CH₂— | 2-NH₂ | 497 |
| 186 | 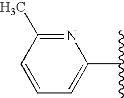 | H | —CH₂— | 2-NH₂ | 511 |
| 187 | 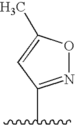 | H | —CH₂— | 3-NH₂ | 501 |
| 188 | 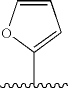 | H | —CH₂— | 2-NH₂ | 486 |
| 189 | 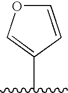 | H | —CH₂— | 2-NH₂ | 486 |
| 190 | 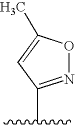 | H | —CH₂— | 2-NH₂ | 501 |
| 191 | 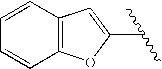 | H | —CH₂— | 2-NH₂ | 536 |
| 192 | 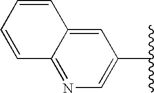 | H | —CH₂— | 2-NH₂ | 547 |
| 193 | 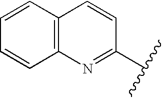 | H | —CH₂— | 2-NH₂ | 547 |
| 194 | 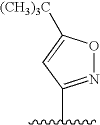 | H | —CH₂— | 2-NH₂ | 543 |
| 195 | 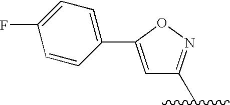 | H | —CH₂— | 2-NH₂ | 581 |
| 196 | 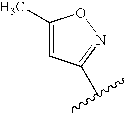 | F | —CH₂— | 2-NH₂ | 519 |
| 197 | 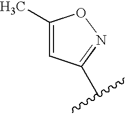 | H | 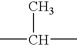 | 2-NH₂ | 515 |
| 198 | 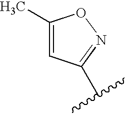 | OH | —CH₂— | 2-NH₂ | 517 |
| 199 | 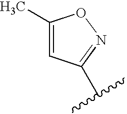 | 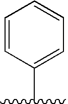 | —CH₂— | 2-NH₂ | 577 |
| 200 | 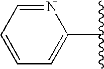 | F | —CH₂— | 2-NH₂ | 515 |
| 201 | 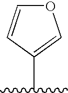 | F | —CH₂— | 2-NH₂ | 504 |
| 202 | 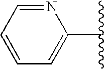 | H | —CH₂— | 3-NH₂ | 497 |
| 203 | 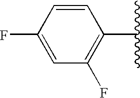 | H | —CH₂— | 3-NH₂ | 532 |
| 204 | 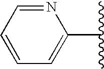 | F | —CH₂— | 3-NH₂ | 515 |
| 205 | 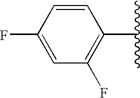 | F | —CH₂— | 3-NH₂ | 550 |

compounds of the following formula:

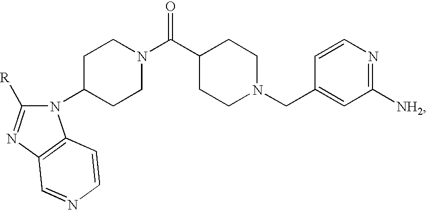

wherein R is as shown in the following table:

| | | |
|---|---|---|
| | | Physical |
| | | Data |
| No. | R | MS (MH⁺) |

| | | |
|---|---|---|
| 206 | —CH₃ | 434 |
| 207 | 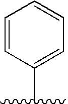 | 497 |
| 208 | 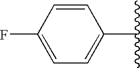 | 514 |
| 209 | 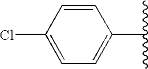 | 530 |

compounds of the following formula:

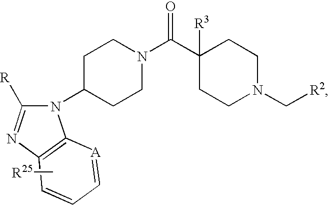

wherein R, R²⁵, A, R³, and R² are as shown in the following table:

| | | | | | | Physical |
|---|---|---|---|---|---|---|
| | | | | | | Data |
| No. | R | R²⁵ | A | R³ | R² | MS (MH⁺) |
| 210 | 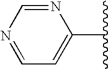 | 5-Cl | C | H | 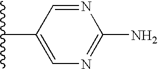 | 532 |
| 211 | 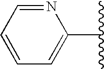 | 5-F | C | H | 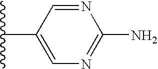 | 515 |
| 212 | 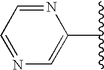 | 5-Cl | C | H | 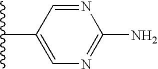 | 532 |
| 213 | 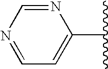 | 5-F | C | H | 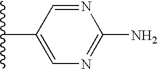 | 516 |
| 214 | 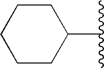 | H | N | H | 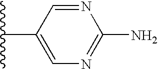 | 503 |
| 215 | 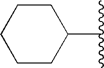 | H | N | H | 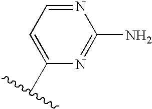 | 503 |
| 216 | (CH₃)₂CH— | H | N | H | 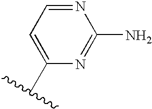 | 463 |
| 217 | 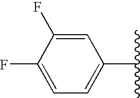 | 5-F | C | H | 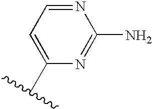 | 550 |
| 218 | 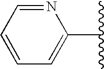 | 5-F | C | H | 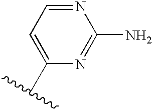 | 515 |
| 219 | 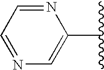 | 5-Cl | C | H | 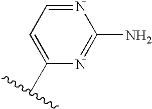 | 532 |
| 220 | 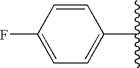 | 6-Cl | C | H | 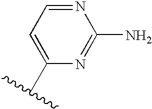 | 548 |
| 221 | 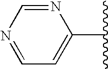 | 5-F | C | H | 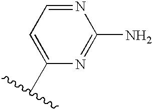 | 516 |
| 222 | 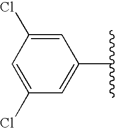 | 6-Cl | C | H | 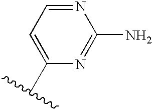 | 600 |
| 223 | 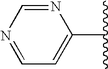 | 5-Cl | C | H | 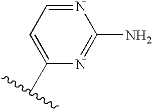 | 532 |
| 224 | 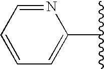 | 6-F | C | H | 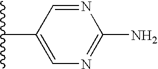 | 515 |
| 225 | 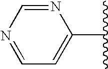 | H | N | H | 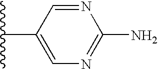 | 499 |
| 226 | 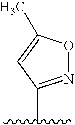 | H | N | H | 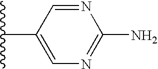 | 502 |
| 227 | 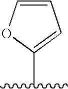 | H | N | H | 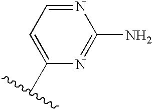 | 487 |
| 228 | 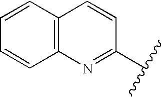 | H | N | H | 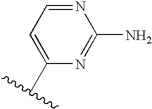 | 548 |
| 229 | 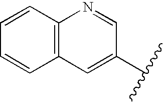 | H | N | H | 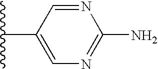 | 548 |
| 230 | 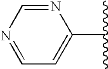 | H | N | H | 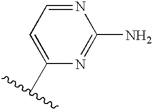 | 499 |
| 231 | 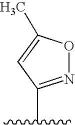 | H | N | H | 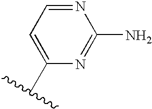 | 502 |
| 232 | 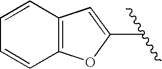 | H | N | H | 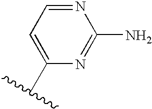 | 537 |
| 233 | 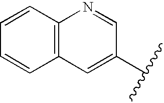 | H | N | H | 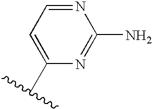 | 548 |
| 234 | 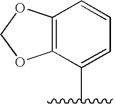 | H | N | H | 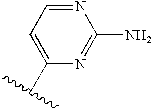 | 541 |
| 235 | 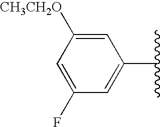 | H | N | H | 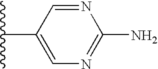 | 559 |
| 236 | 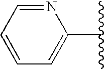 | H | N | H | 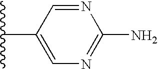 | 498 |
| 237 | 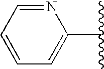 | 5-F | C | F | 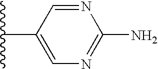 | 533 |
| 238 | 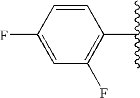 | 5-F | C | H | 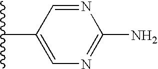 | 550 |
| 239 | 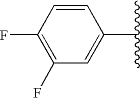 | 5-F | C | H | 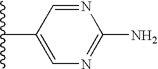 | 550 |
| 240 | 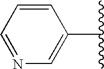 | 5-F | C | H | 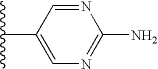 | 515 |
| 241 | 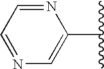 | 5-F | C | H | 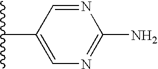 | 516 |
| 242 | 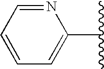 | H | C | H | 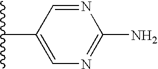 | 497 |
| 243 | (CH₃)₂N—CH₂— | H | N | H | 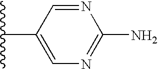 | 478 |
| 244 | 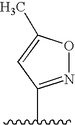 | 5-F | C | H | 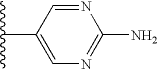 | 519 |
| 245 | 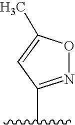 | H | C | H | 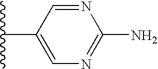 | 501 |
| 246 | 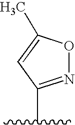 | 5,6-di-F | C | H | 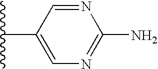 | 537 |
| 247 | 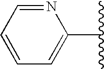 | 5-F | C | H | 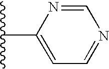 | 500 |
| 248 | 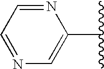 | 5,6-di-F | C | H | 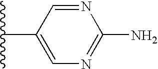 | 534 |
| 249 | 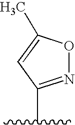 | 5-F | C | F | 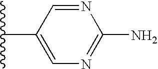 | 537 |
| 250 | 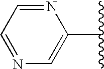 | 5-F | C | F | 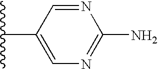 | 534 |
| 251 | 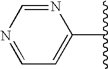 | 5-F | C | F | 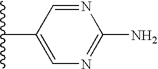 | 534 |
| 252 | 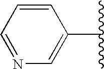 | 5-F | C | F | 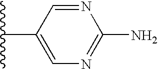 | 533 |
| 253 | 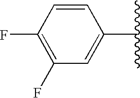 | 5-F | C | F | 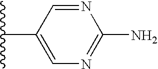 | 568 |
| 254 | 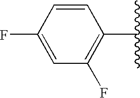 | 5-F | C | F | 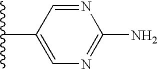 | 568 |
| 255 | 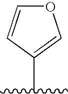 | H | N | H | 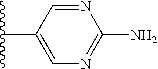 | 487 |
| 256 | 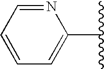 | H | C | F | 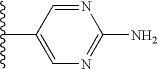 | 515 |
| 257 | 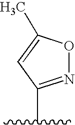 | H | C | F | 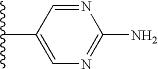 | 519 |
| 258 | 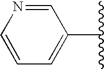 | H | N | F | 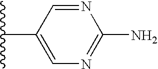 | 516 |
| 259 | 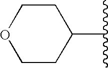 | H | N | H | 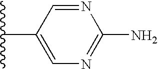 | 505 |
| 260 | 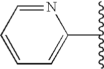 | H | N | F | 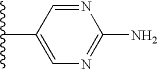 | 516 |
| 261 | 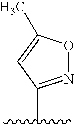 | H | N | F | 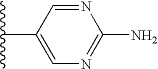 | 520 |
| 262 | 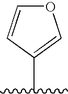 | 5-F | C | H | 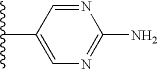 | 504 |
| 263 | 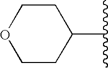 | 5-F | C | H | 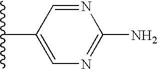 | 522 |
| 264 | 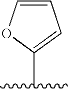 | 5-F | C | H | 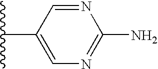 | 504 |
| 265 | 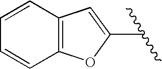 | H | N | H | 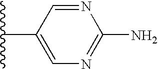 | 537 |
| 266 | (CH₃)₂N—CH₂— | H | N | 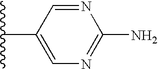 | 496 | |
| 267 | 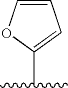 | H | N | F | 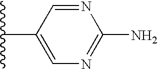 | 505 |
| 268 | CH₃CH₂—O— | 5-F | C | H | 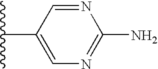 | 482 |
| 269 | CH₃—S— | 5-F | C | H | 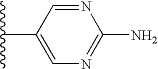 | 484 |
| 270 | CH₃CH₂—O— | 5-F | C | F | 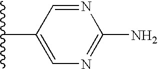 | 500 |
| 271 | 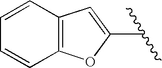 | H | N | F | 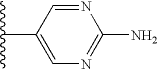 | 555 |
| 272 | 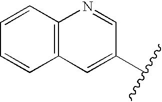 | H | N | F | 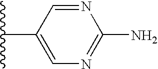 | 566 |
| 273 | 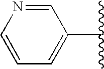 | H | N | H | 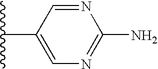 | 498 |
| 274 | 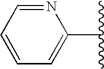 | 5,6-di-F | C | F | 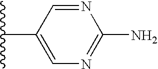 | 551 |
| 275 | 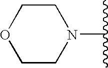 | 5-F | C | F | 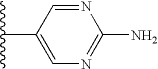 | 541 |
| 276 | 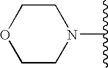 | 5-F | C | H | 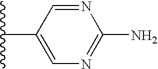 | 523 |
| 277 | 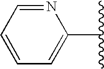 | 5-F | C | H | 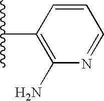 | 514 |
| 278 | 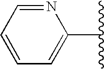 | 5-F | C | H | 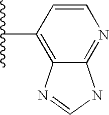 | 539 |
| 279 | 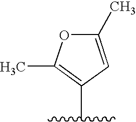 | H | N | H | 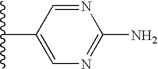 | 515 |
| 280 | 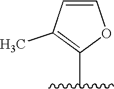 | H | N | H | 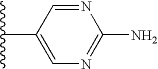 | 501 |
| 281 | 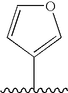 | H | N | F | 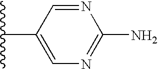 | 505 |
| 282 | 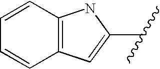 | H | N | H | 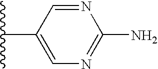 | 536 |
| 283 | 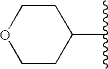 | H | N | F | 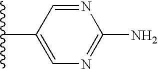 | 523 |
| 284 | 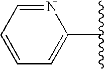 | 5-F | C | F | 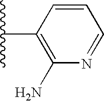 | 532 |
| 285 | 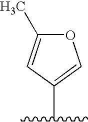 | H | N | H | 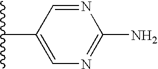 | 501 |
| 286 | 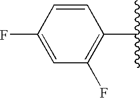 | H | N | H | 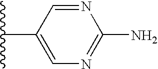 | 533 |
| 287 | 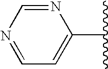 | H | N | F | 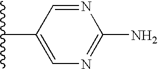 | 517 |
| 288 | 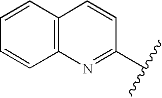 | H | N | H | 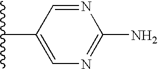 | 548 |
| 289 | 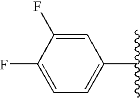 | H | N | H | 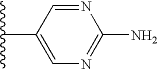 | 533 |
| 290 | CH₃S— | 5-F | C | F | 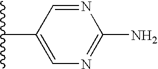 | 502 |
| 291 | 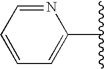 | H | N | F | 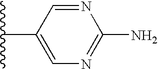 | 515 |
| 292 | 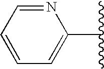 | 5-F | C | F | 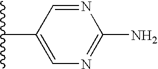 | 532 |
| 293 | 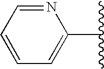 | 5-F | C | H | 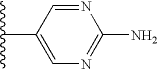 | 514 |
| 294 | 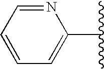 | H | N | H | 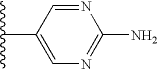 | 497 |
| 295 | (CH₃)₂N— | 5-F | C | F | 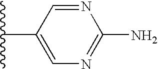 | 499 |
| 296 | CH₃CH₂—S— | 5-F | C | F | 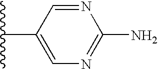 | 516 |
| 297 | CH₃—O— | 5-F | C | F | 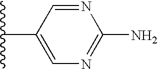 | 486 |
| 298 | 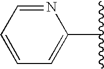 | H | N | H | 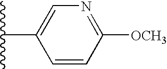 | 512 |
| 299 | 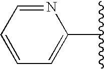 | H | N | F | 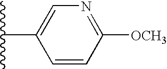 | 530 |
| 300 | 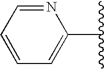 | 5-F | C | F | 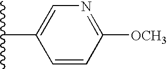 | 547 |
| 301 | 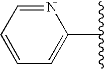 | 5-F | C | H | 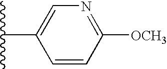 | 529 |
| 302 | 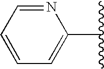 | 5-F | C | H | 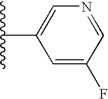 | 517 |
| 303 | 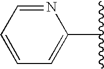 | 5-F | C | F | 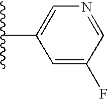 | 535 |
| 304 | 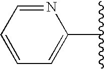 | H | N | H | 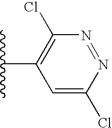 | 551 |
| 305 | 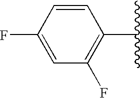 | H | N | F | 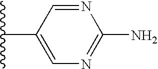 | 551 |
| 306 | 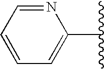 | 5-F | C | H | 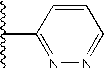 | 500 |
| 307 | 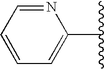 | 5-F | C | H | 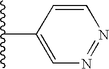 | 500 |
| 308 | 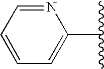 | 5-F | C | F | 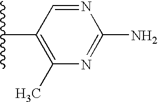 | 547 |
| 309 | (CH₃CH₂)₂N— | 5-F | C | F | 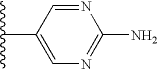 | 527 |
| 310 | 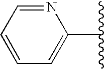 | H | N | H | 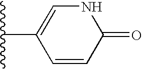 | 498 |
| 311 | 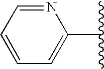 | H | N | F | 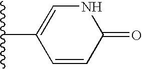 | 516 |
| 312 | 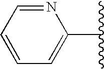 | 5-F | C | H | 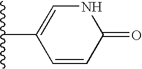 | 515 |
| 313 | 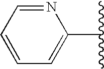 | 5-F | C | F | 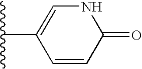 | 533 |
| 314 | 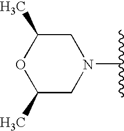 | 5-F | C | F | 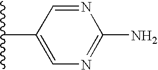 | 569 |
| 315 | CH₃—S— | H | N | F | 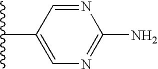 | 485 |
| 316 | CH₃CH₂—O— | H | N | F | 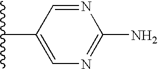 | 483 |
| 317 | 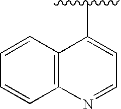 | H | N | F | 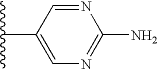 | 566 |
| 318 | 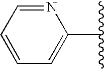 | H | N | F | 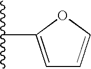 | 489 |
| 319 | 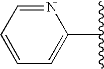 | H | N | F | 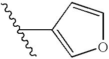 | 489 |
| 320 | 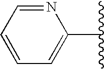 | H | N | F | 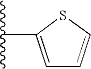 | 505 |
| 321 | 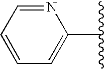 | H | N | F | 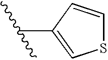 | 505 |
| 322 | 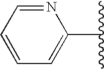 | 5-F | C | F | 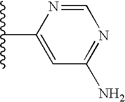 | 533 |
| 323 | 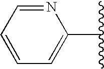 | H | N | F | 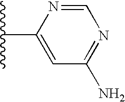 | 516 |
| 325 | 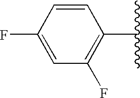 | H | N | F | 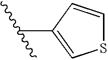 | 540 |
| 325 | 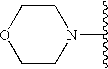 | H | N | F | 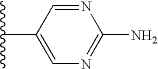 | 524 |
| 326 | (CH₃)₂CH—O— | 5-F | C | F | 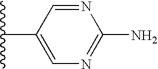 | 514 |
| 327 | 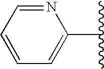 | H | N | F | 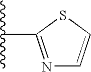 | 506 |
| 328 | 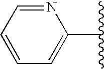 | H | N | F | 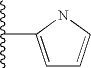 | 488 |
| 329 | 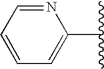 | H | N | F | 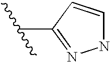 | 489 |
| 330 | 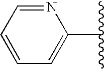 | H | N | F | 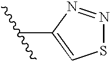 | 507 |
| 331 | 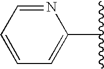 | H | N | F | 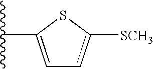 | 551 |
| 332 | 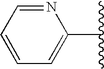 | H | N | F | 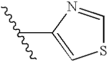 | 506 |
| 333 | 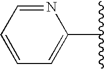 | H | N | F | 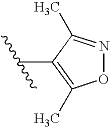 | 518 |
| 334 | 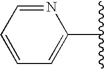 | H | N | F | 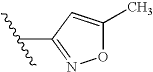 | 504 |
| 335 | CH₃—O— | H | N | F | 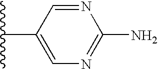 | 464 |
| 336 | 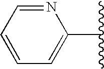 | H | N | F | 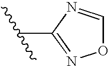 | 491 |
| 337 | 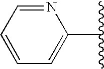 | H | N | F | 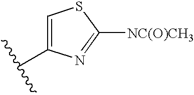 | 563 |
| 338 | 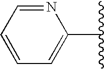 | 5-F | C | H | 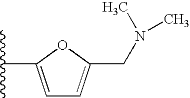 | 545 |
| 339 | 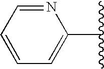 | 5-F | C | F | 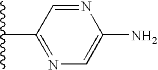 | 533 |
| 340 | 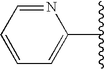 | H | N | F | 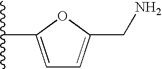 | 518 |
| 341 | 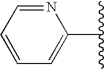 | 5-F | C | H | 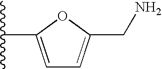 | 535 |
| 342 | 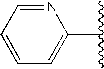 | H | N | F | 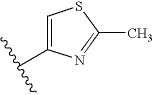 | 520 |
| 343 | 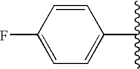 | 6-Cl | C | H | 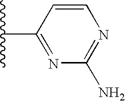 | 548 |
| 345 | 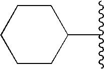 | H | N | H | 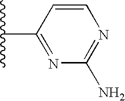 | 503 |
| 346 | (CH₃)₂—CH— | H | N | H | 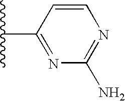 | 436 |

compounds of the following formula:

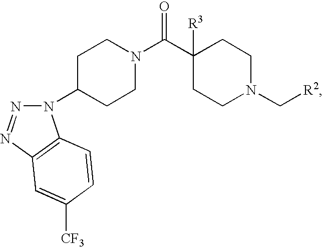

wherein R³ and R² shown in the following table:

| | | | Physical |
|---|---|---|---|
| | | | Data |
| No. | R³ | R² | MS (MH⁺) |
| 347 | H | 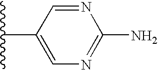 | 489 |
| 348 | F | 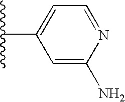 | 506 |
| 349 | F | 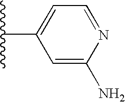 | 488 |
| 350 | F | 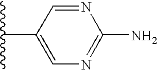 | 507 |
| 351 | F | 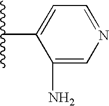 | 506 |

compounds of the following formula:

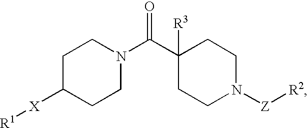

wherein R¹—X—, Z, R³, and R² are as shown in the following table:

| | | | | | Physical |
|---|---|---|---|---|---|
| | | | | | Data |
| No. | R¹—X— | Z | R³ | R² | MS (MH⁺) |
| 361 | 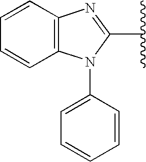 | —CH₂— | H | 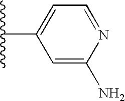 | 495 |
| 362 | 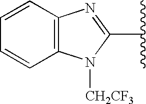 | —CH₂— | H | 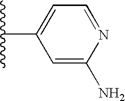 | 501 |
| 363 | 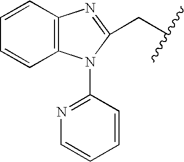 | —CH₂— | H | 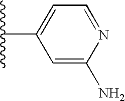 | 510 |
| 364 | 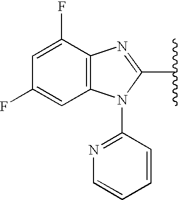 | —CH₂— | H | 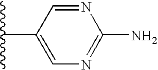 | 533 |
| 365 | 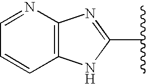 | —CH₂— | H | 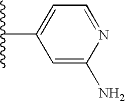 | 420 |
| 366 | 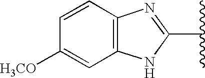 | —CH₂— | H | 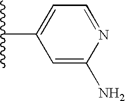 | 449 |
| 367 | 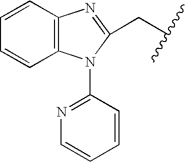 | —CH₂— | H | 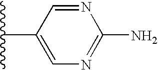 | 497 |
| 368 | 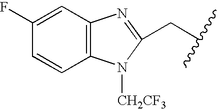 | —CH₂— | H | 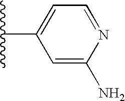 | 533 |
| 369 | 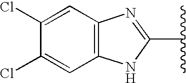 | —CH₂— | H | 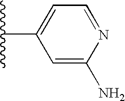 | 487 |
| 370 | 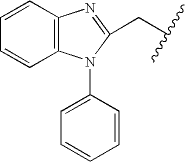 | —CH₂— | H | 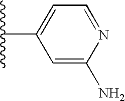 | 509 |
| 371 | 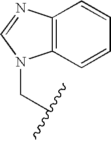 | —CH₂— | H | 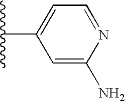 | 433 |
| 372 | 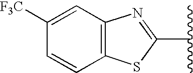 | —CH₂— | H | 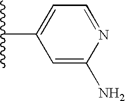 | 504 |
| 373 | 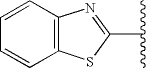 | —CH₂— | H | 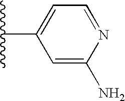 | 436 |
| 374 | 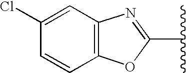 | —CH₂— | H | 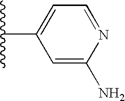 | 472 |
| 375 | 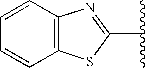 | —(CH₂)₃— | H | 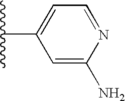 | 464 |
| 376 | 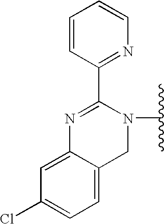 | —CH₂— | H | 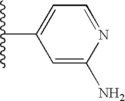 | 544 |
| 377 | 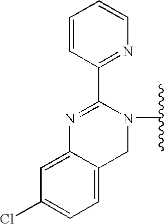 | —CH₂— | F | 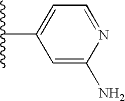 | 562 |

compounds of the following formula:

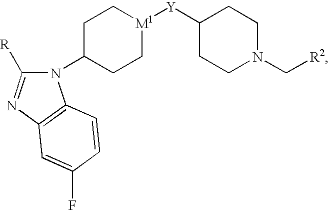

wherein R, M¹, Y, and R² are as shown in the following table:

| | | | | | Physical |
|---|---|---|---|---|---|
| | | | | | Data |
| No. | R | M¹ | Y | R² | MS (MH⁺) |
| 378 | 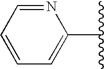 | CH | —CH₂— | 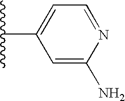 | 500 |
| 379 | 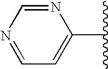 | N | —NH— | 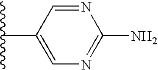 | 502 |
| 380 | 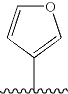 | N | —NH— | 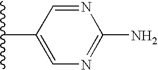 | 490 |
| 381 | 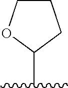 | N | —NH— | 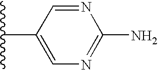 | 494 |
| 382 | 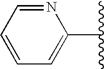 | N | —NH— | 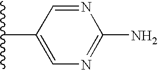 | 501 |
| 383 | 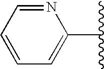 | N | —NH— | 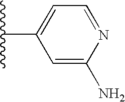 | 500 |

and

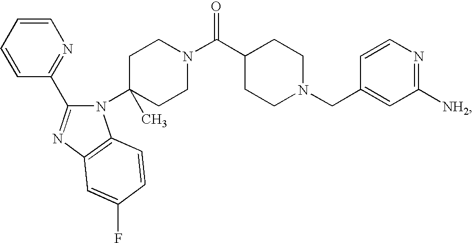

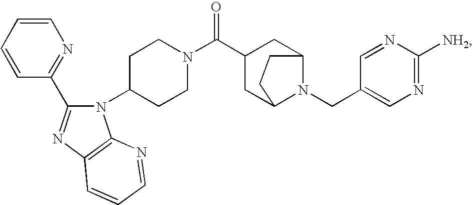

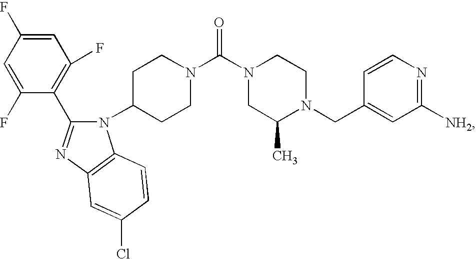

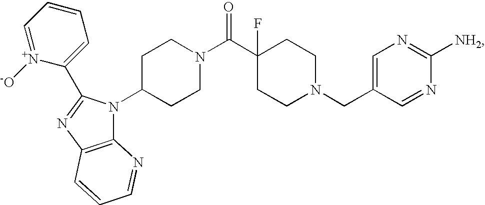

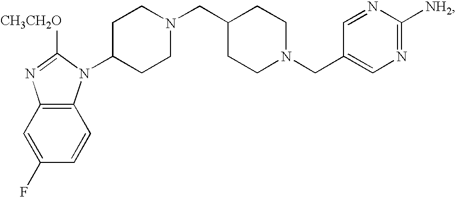

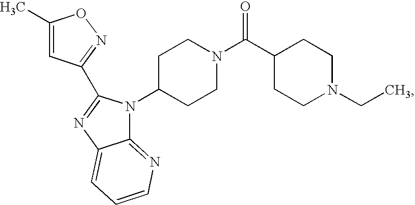

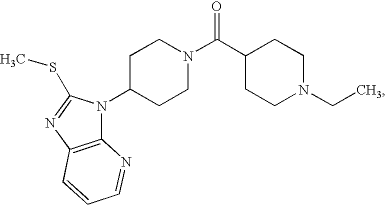

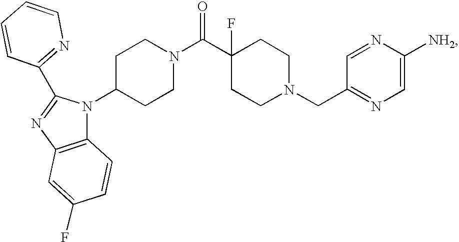

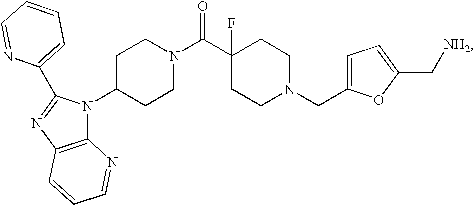

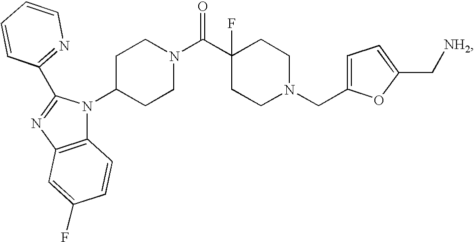

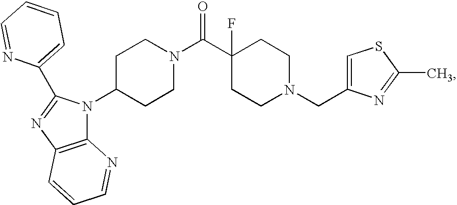

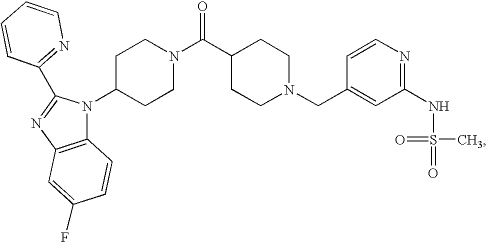

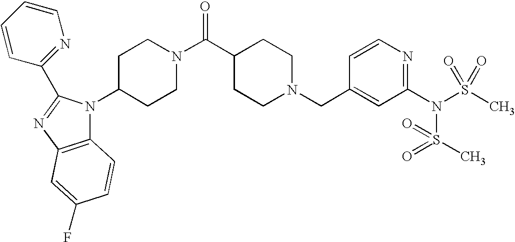

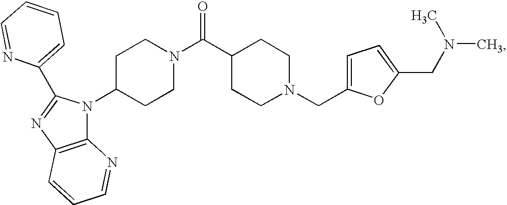

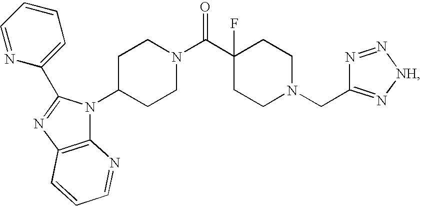

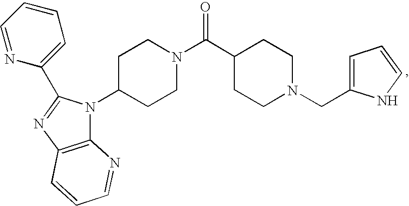

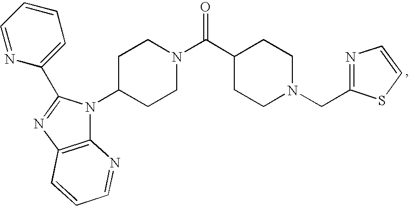

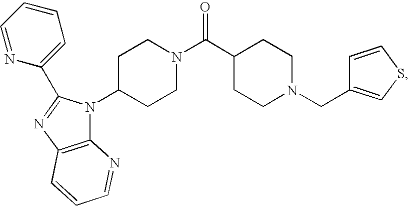

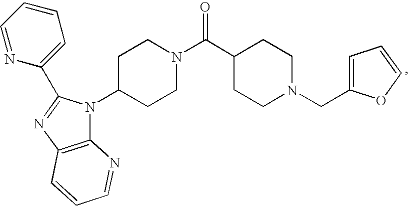

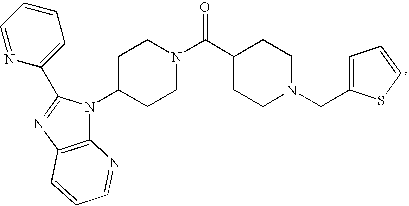

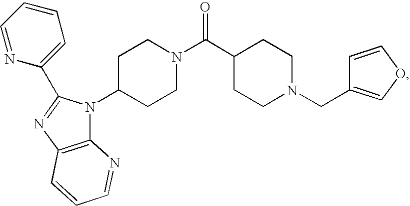

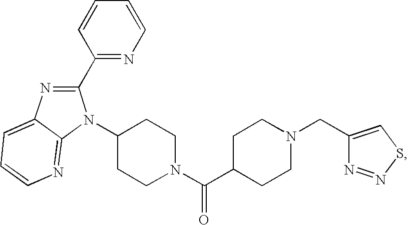

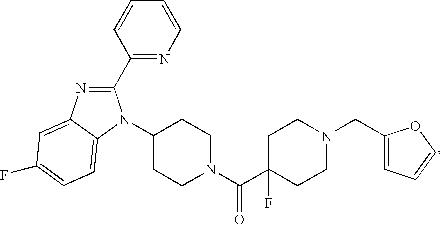

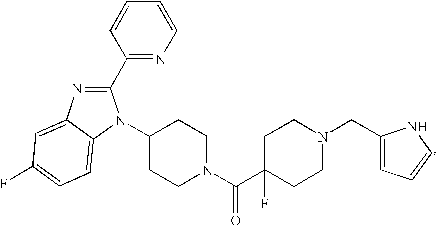

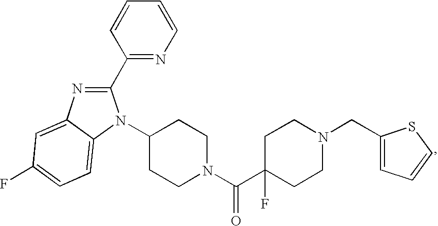

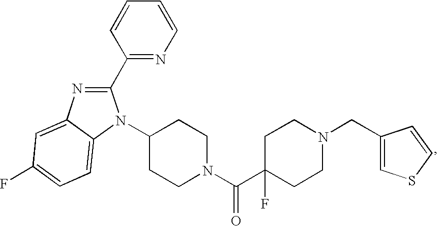

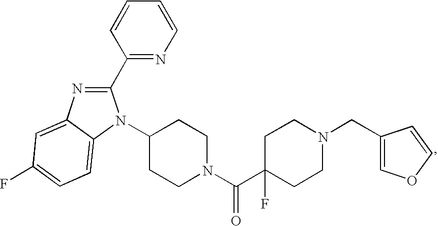

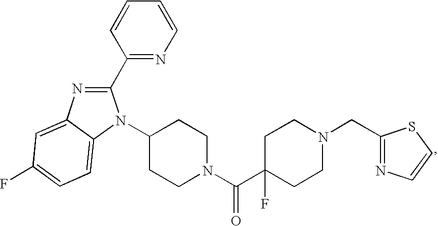

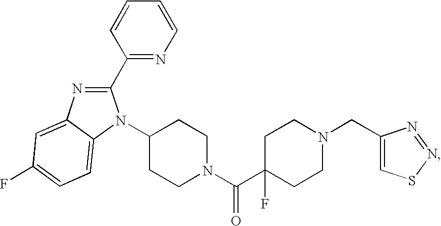

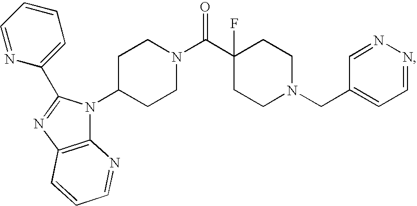

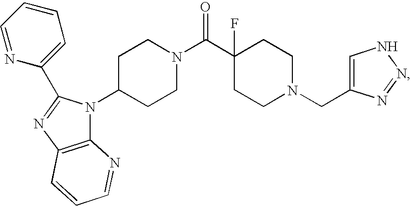

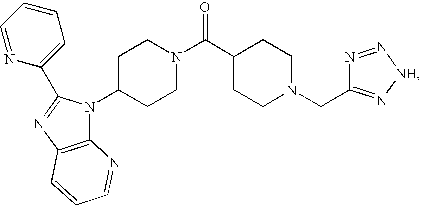

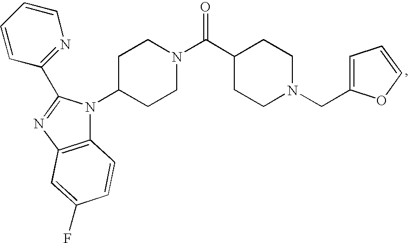

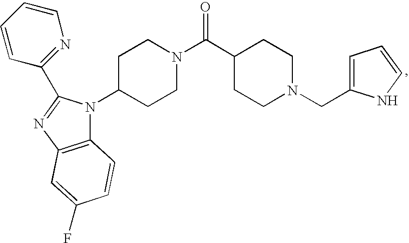

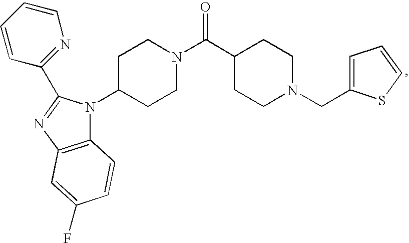

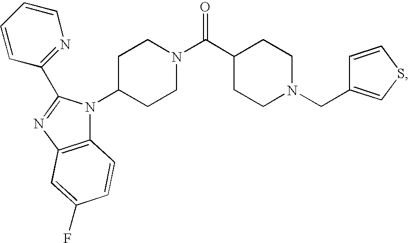

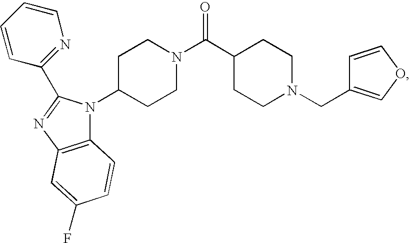

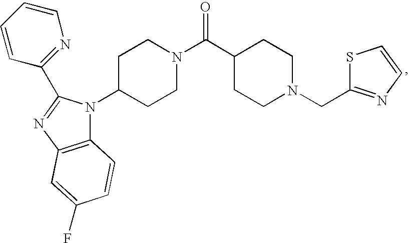

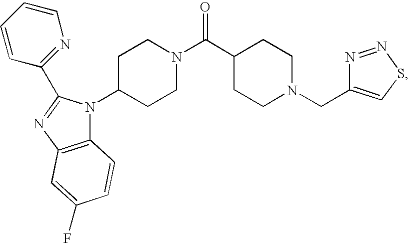

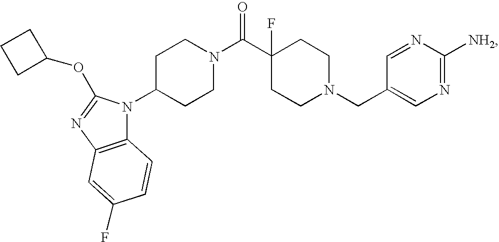

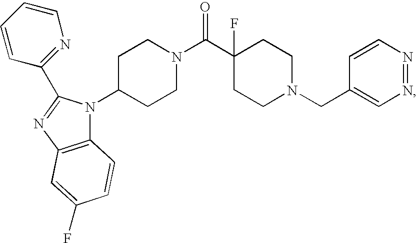

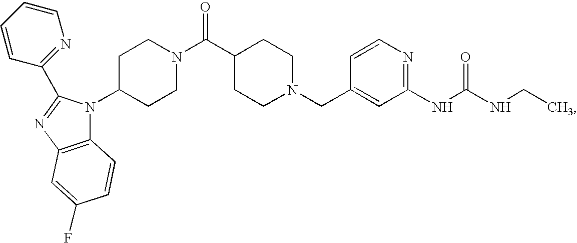

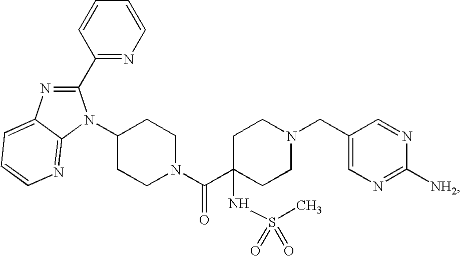

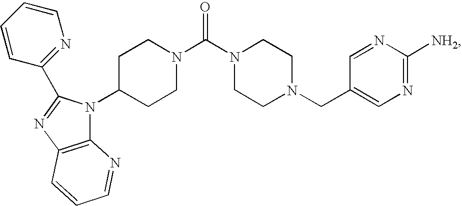

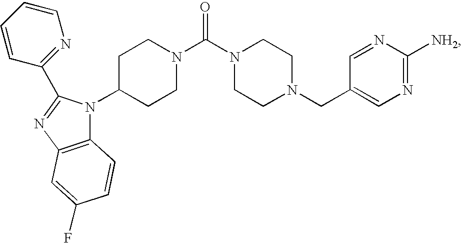

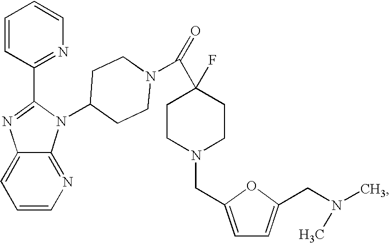

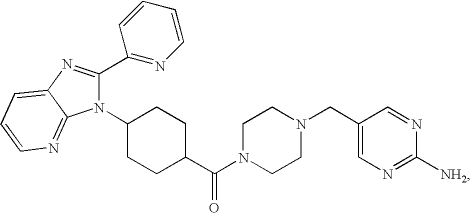

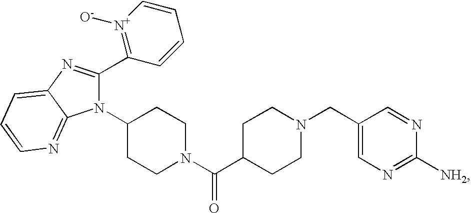

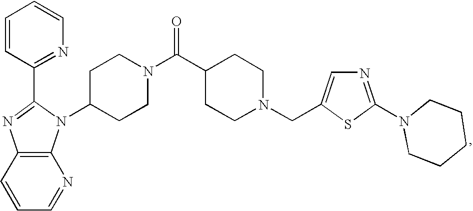

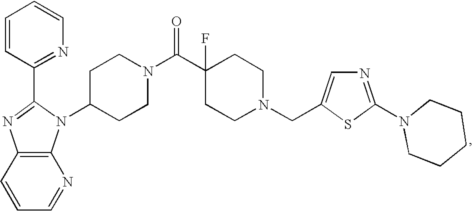

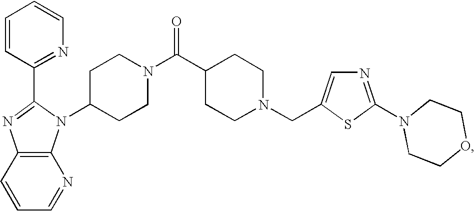

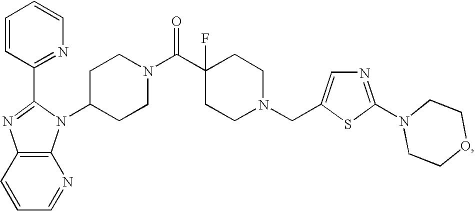

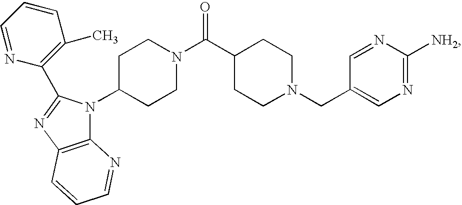

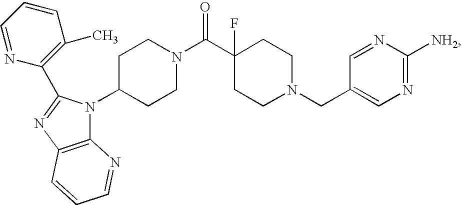

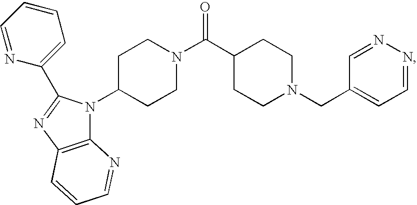

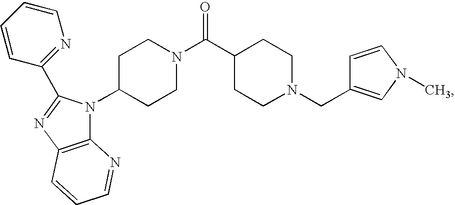

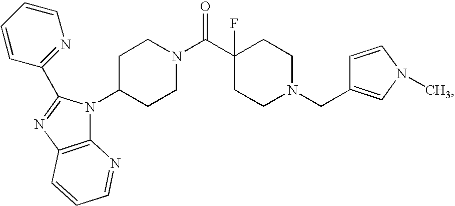

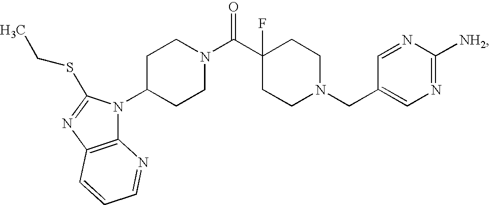

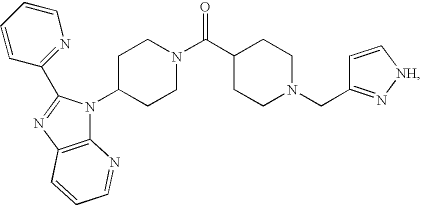

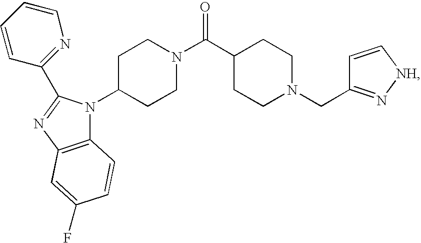

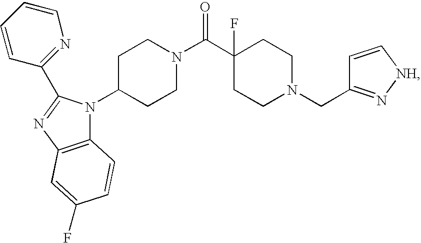

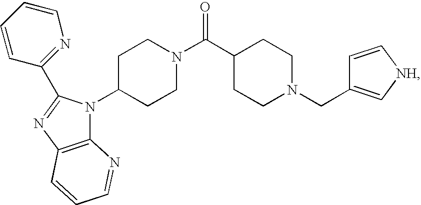

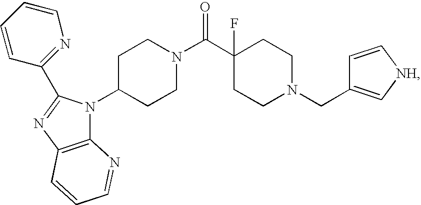

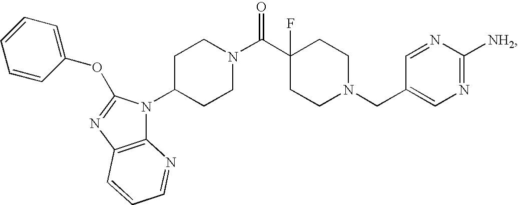

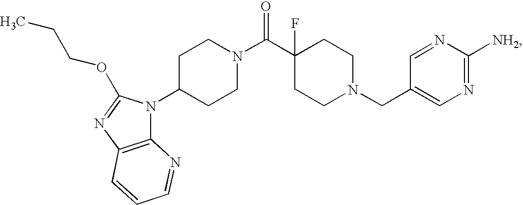

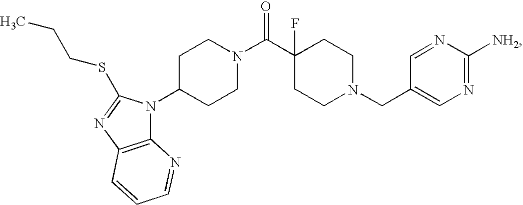

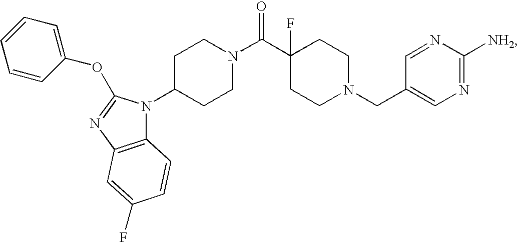

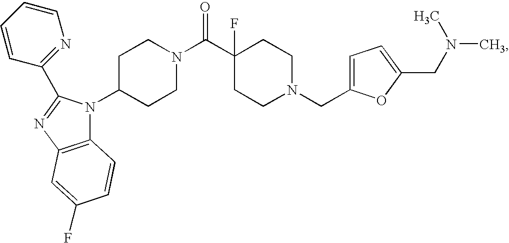

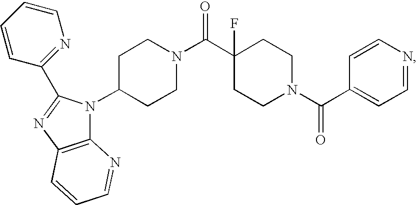

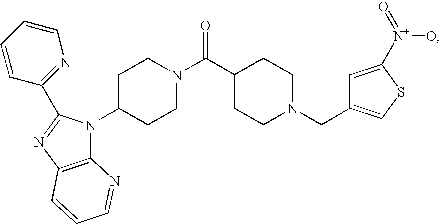

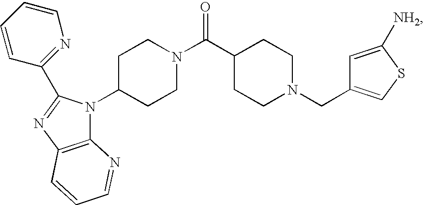

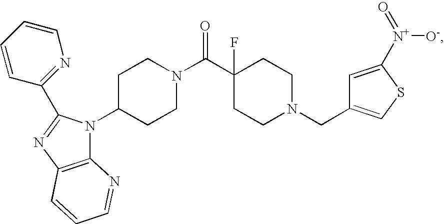

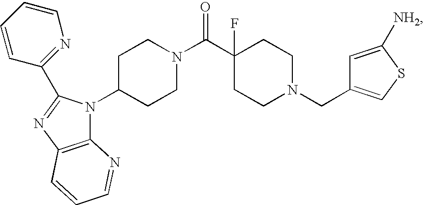

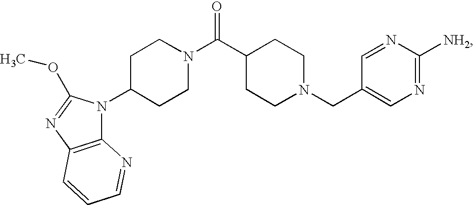

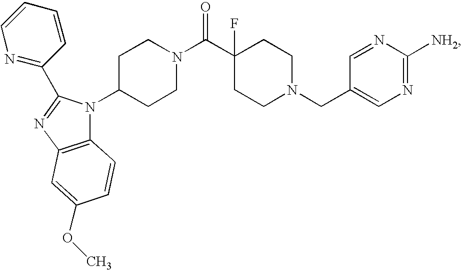

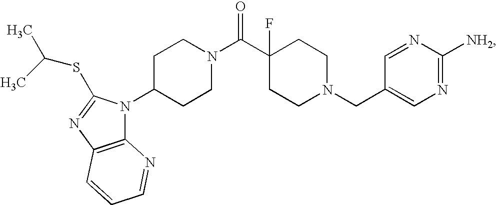

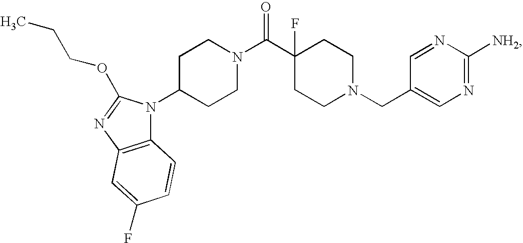

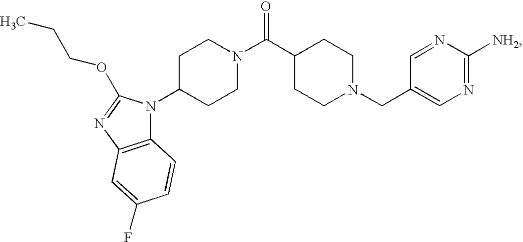

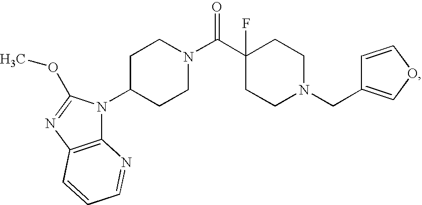

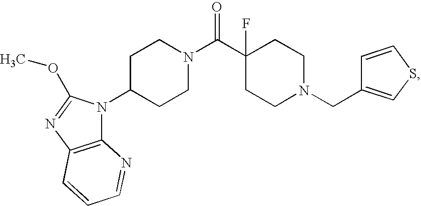

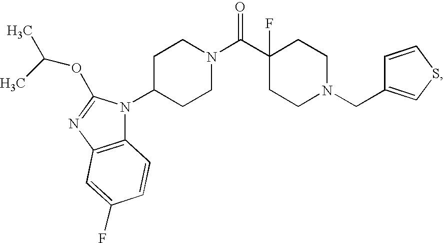

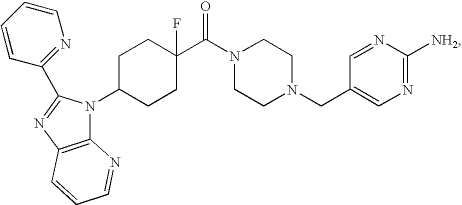

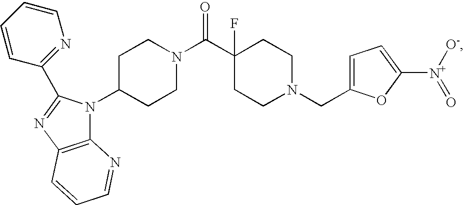

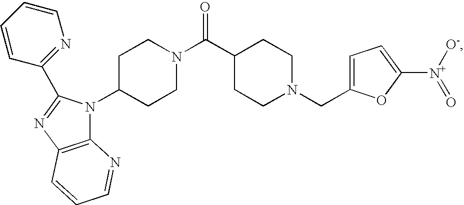

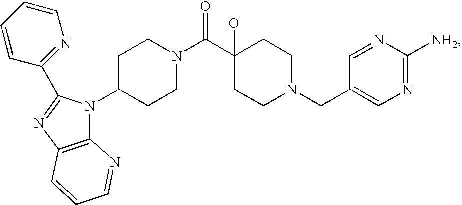

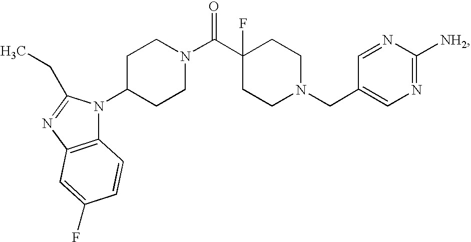

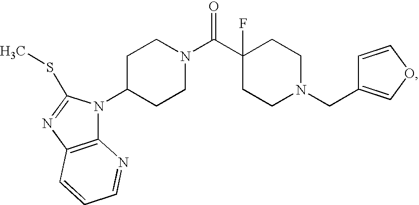

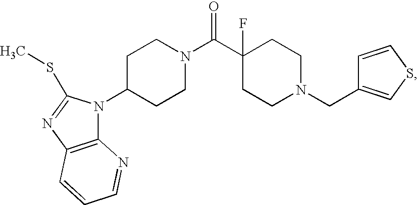

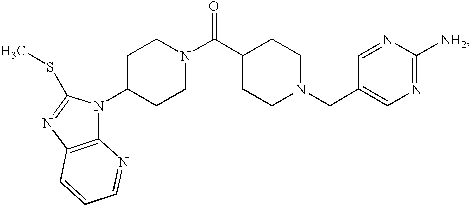

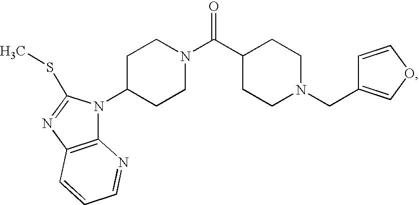

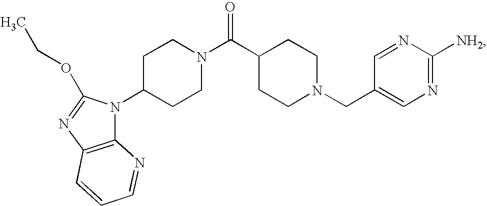

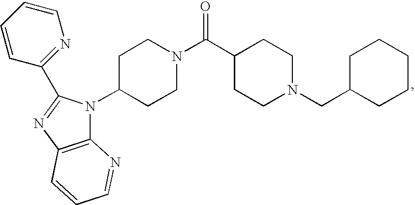

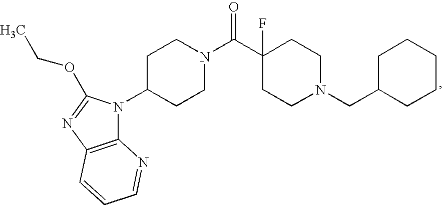

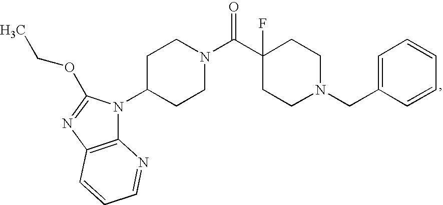

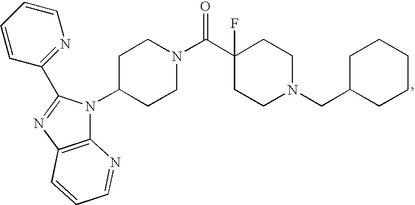

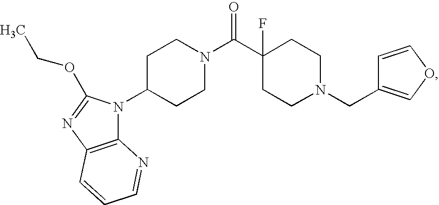

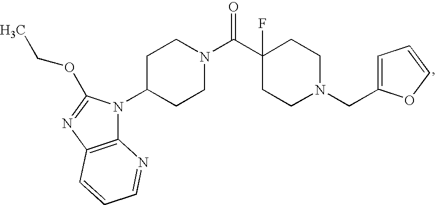

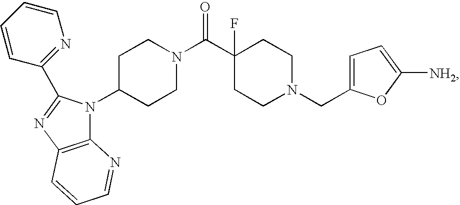

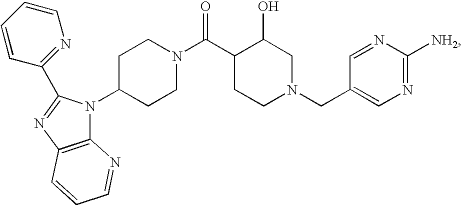

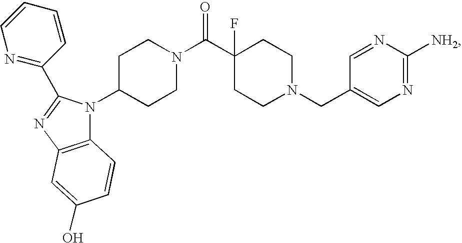

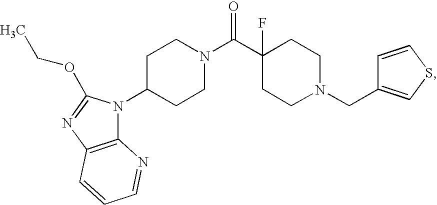

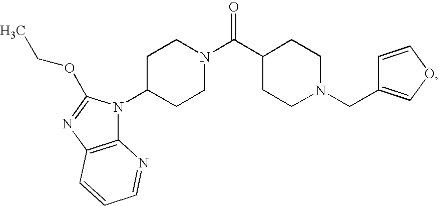

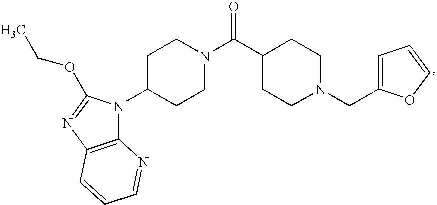

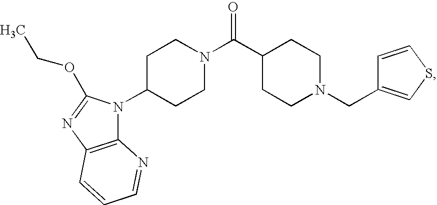

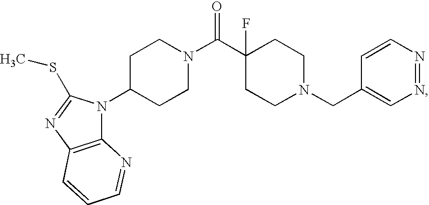

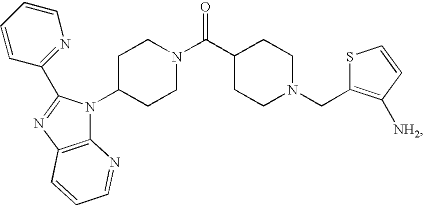

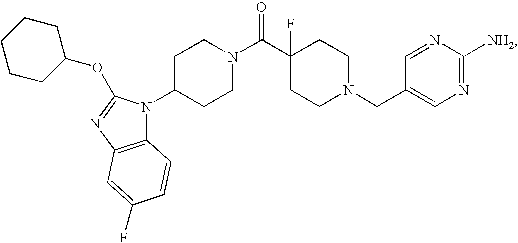

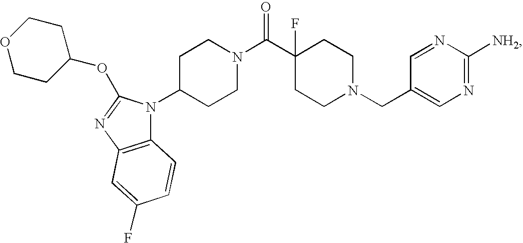

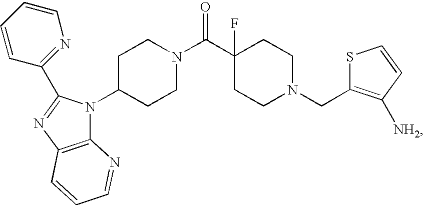

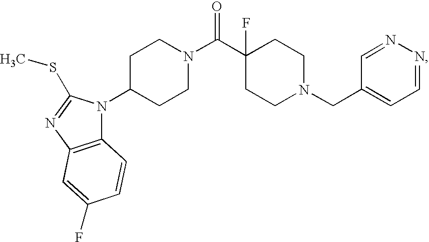

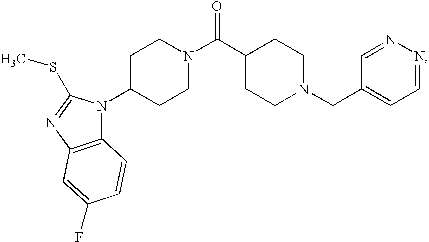

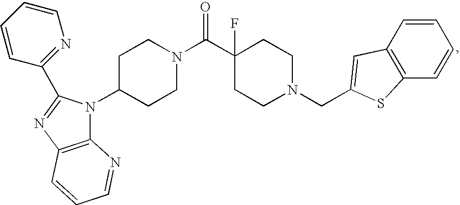

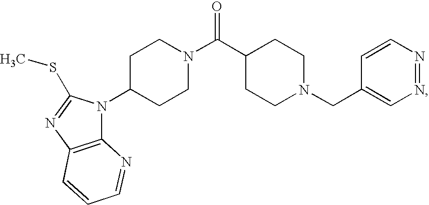

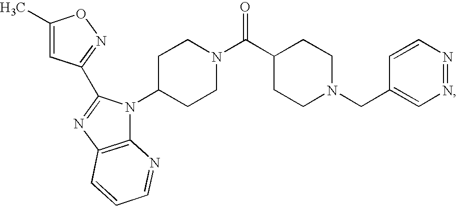

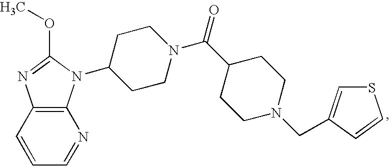

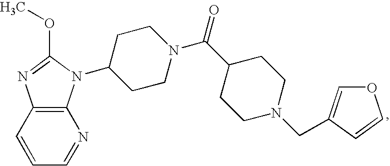

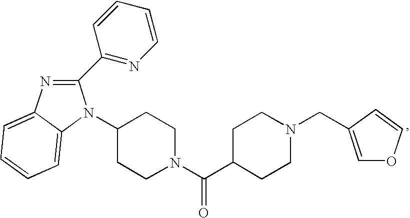

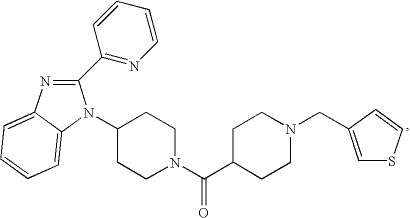

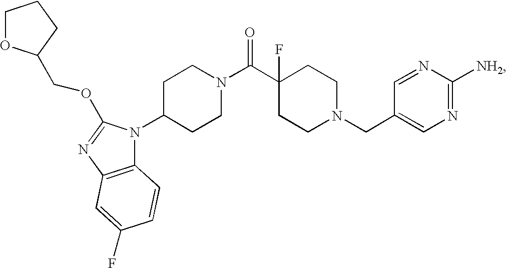

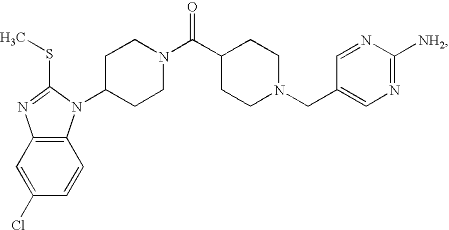

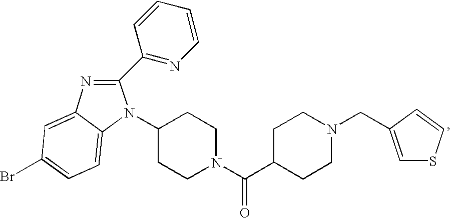

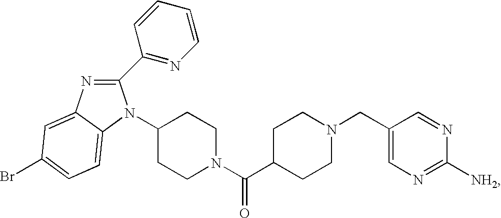

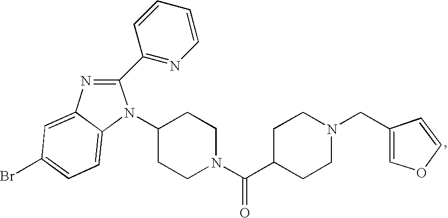

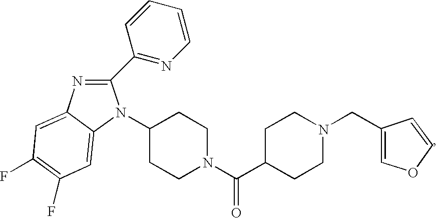

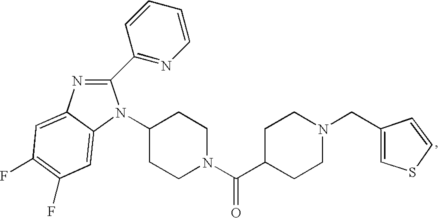

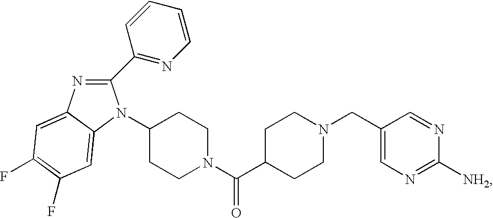

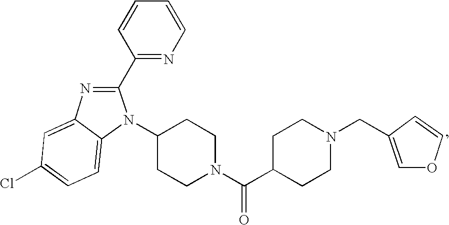

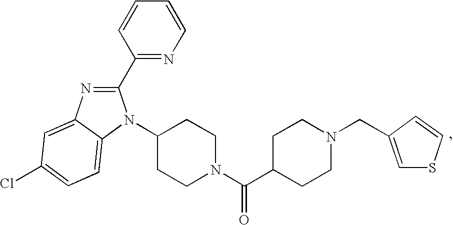

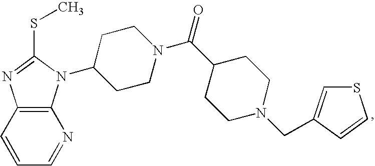

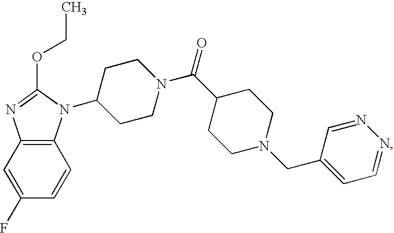

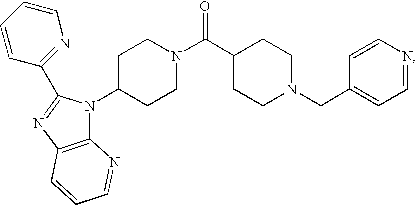

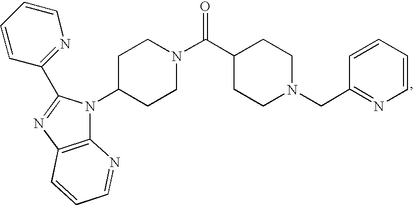

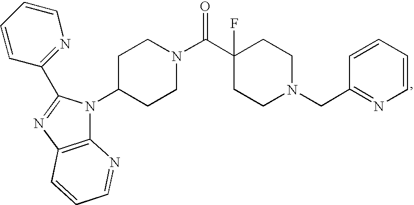

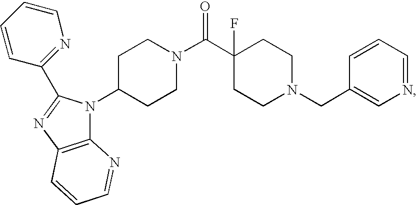

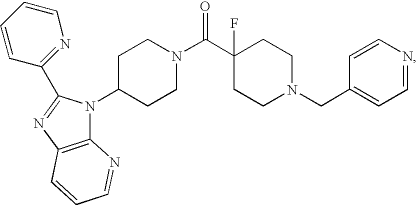

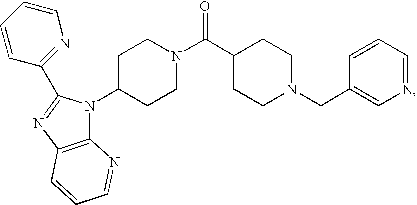

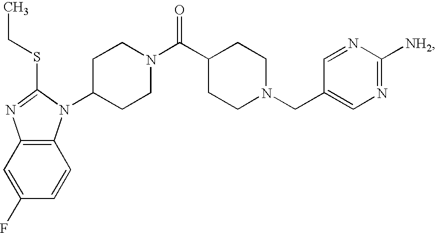

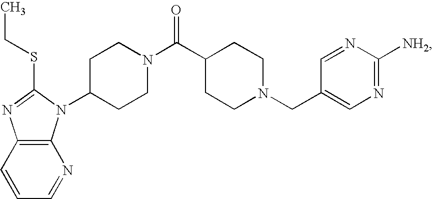

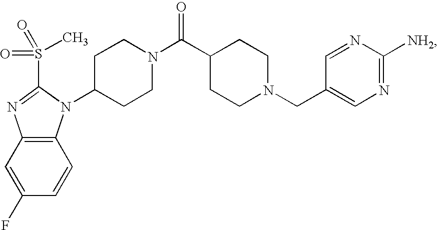

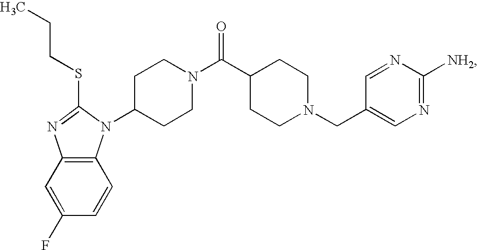

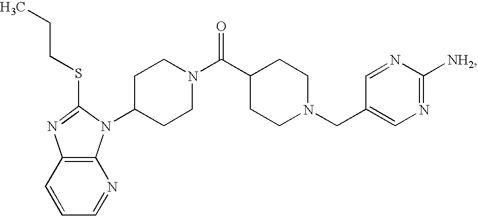

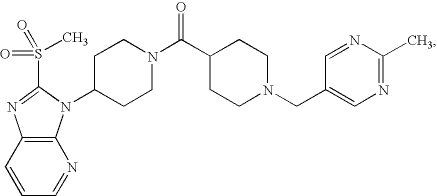

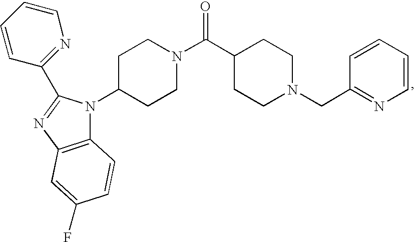

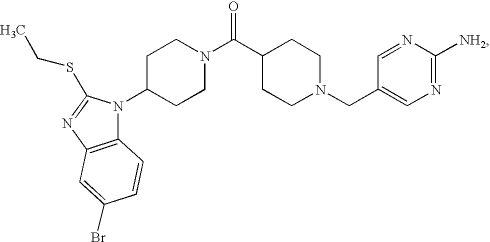

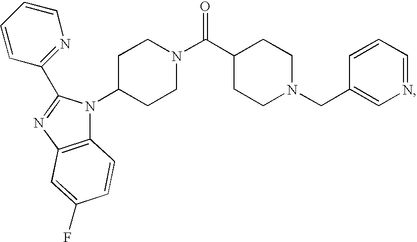

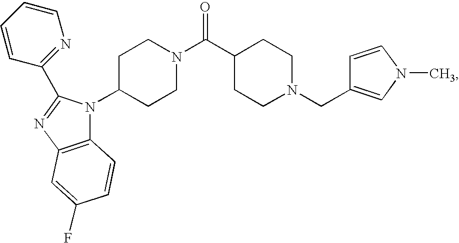

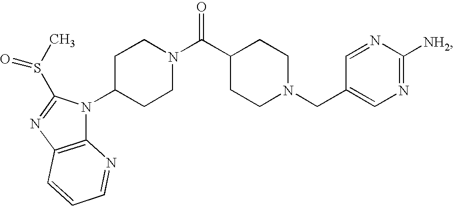

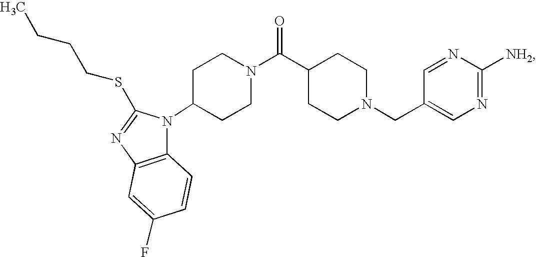

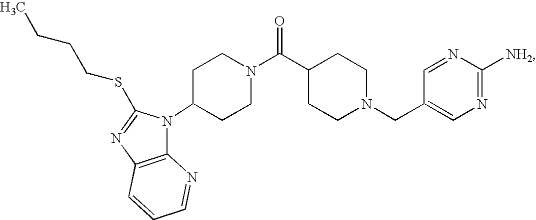

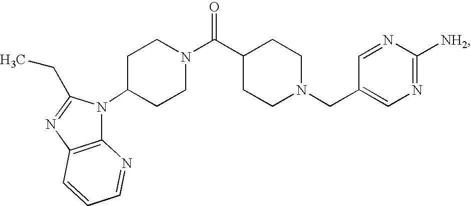

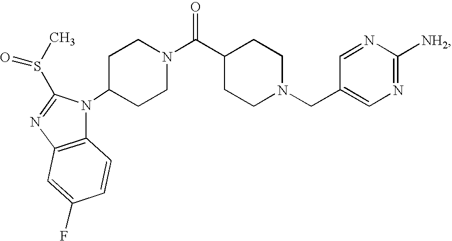

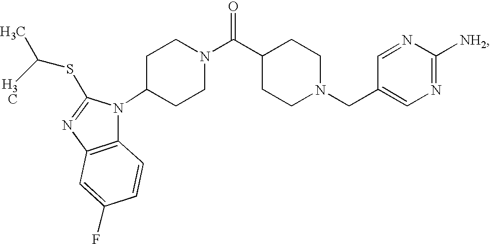

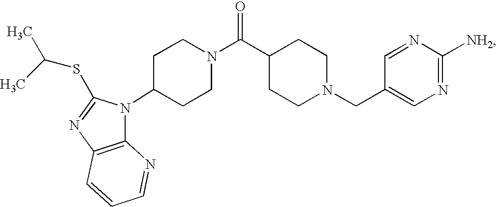

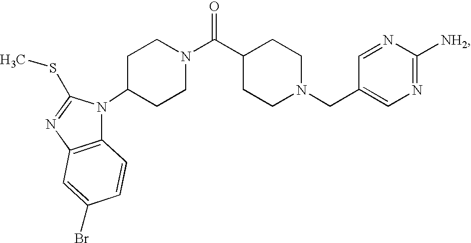

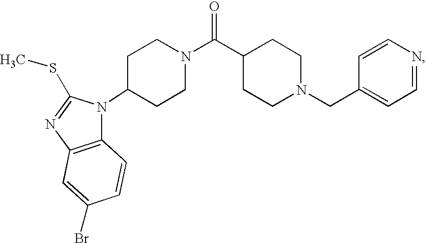

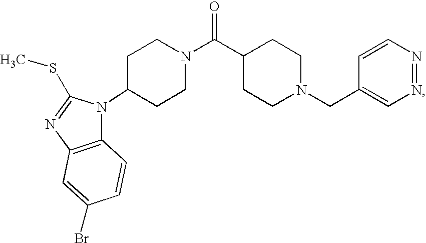

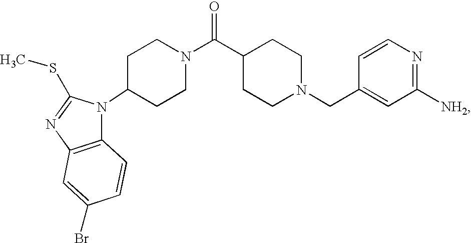

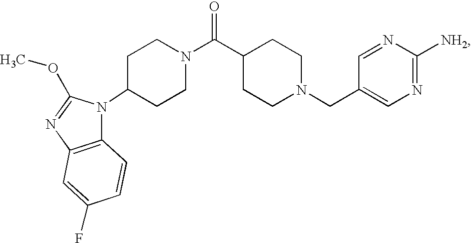

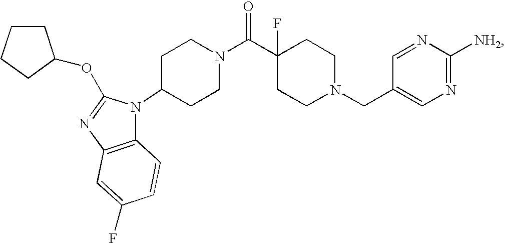

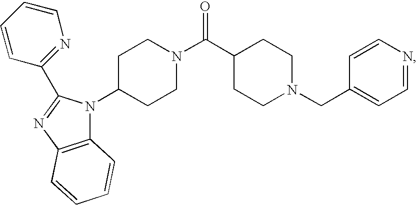

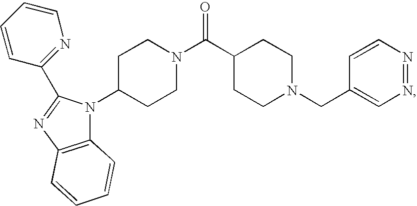

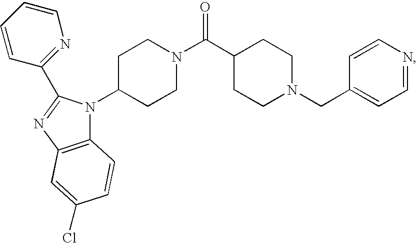

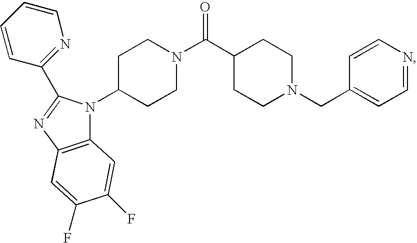

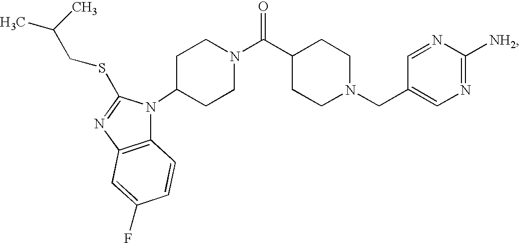

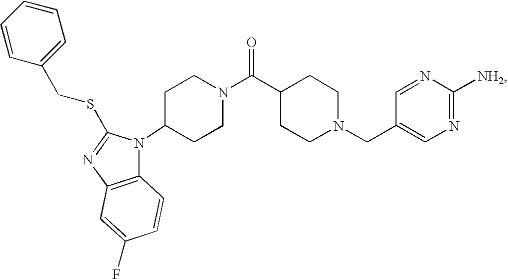

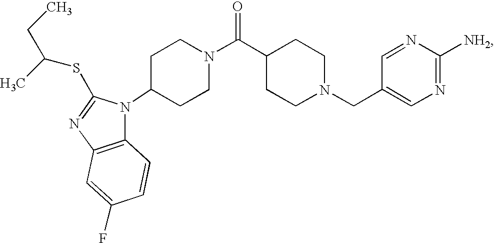

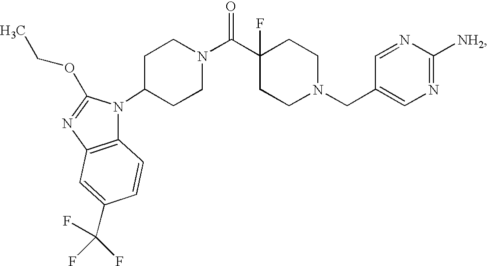

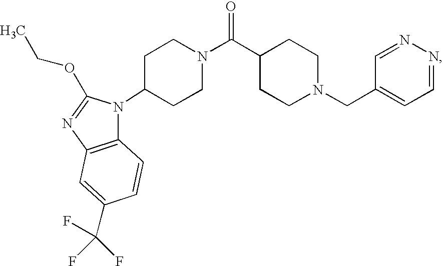

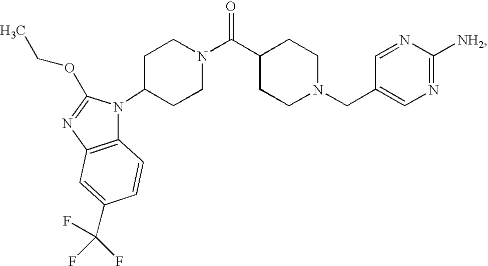

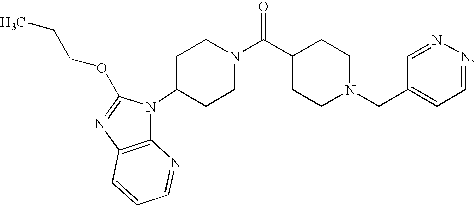

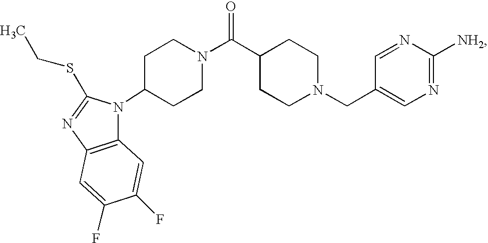

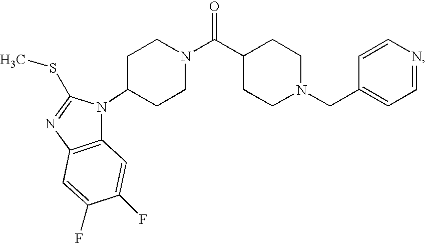

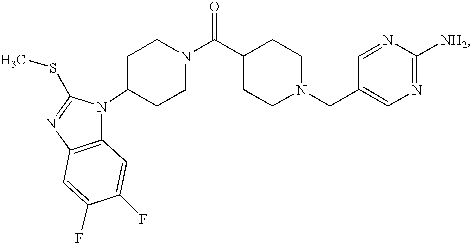

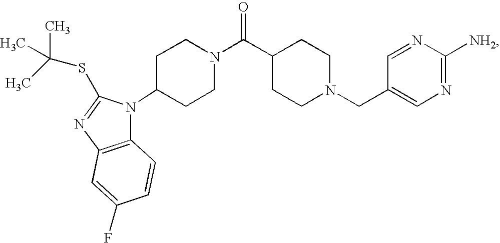

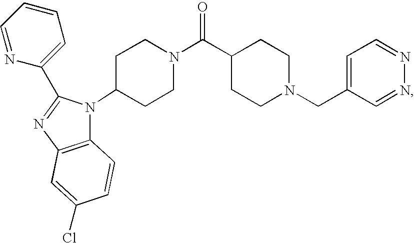

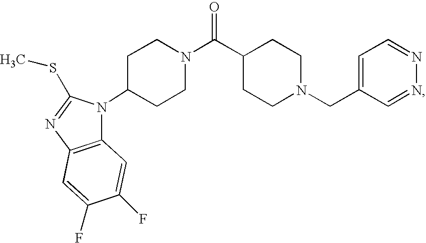

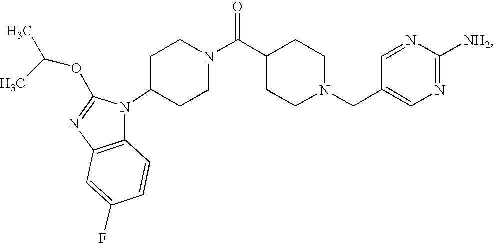

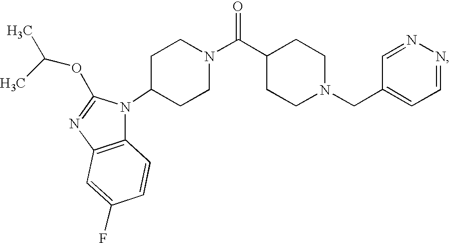

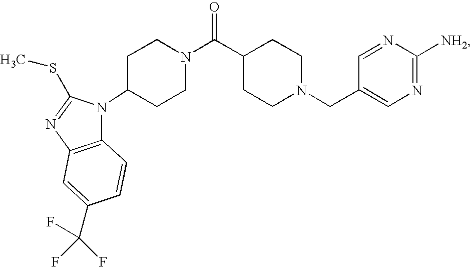

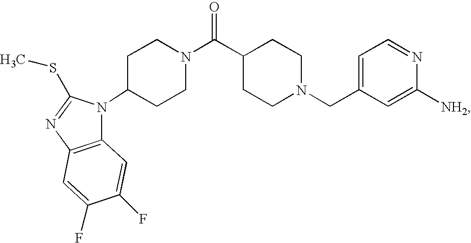

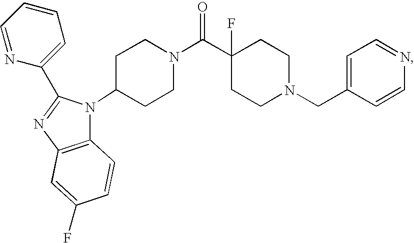

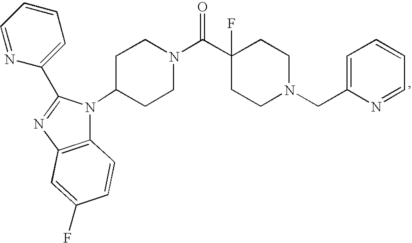

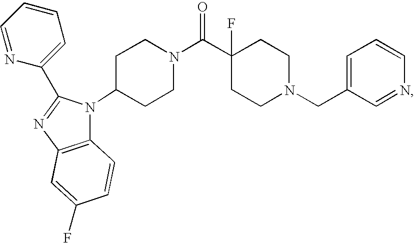

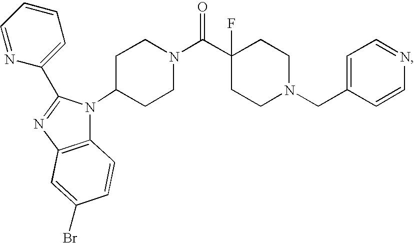

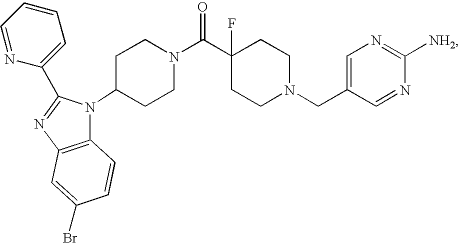

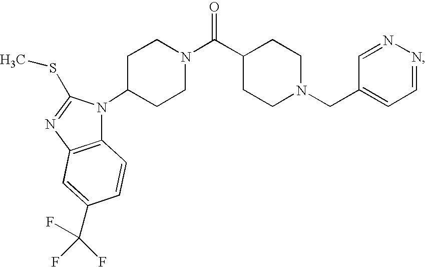

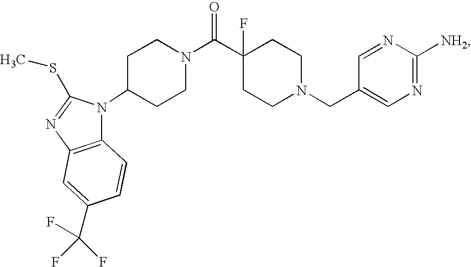

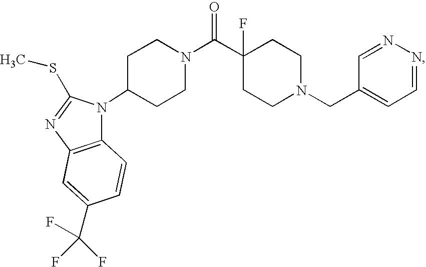

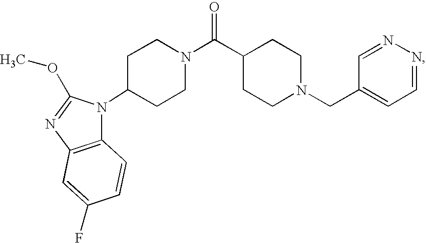

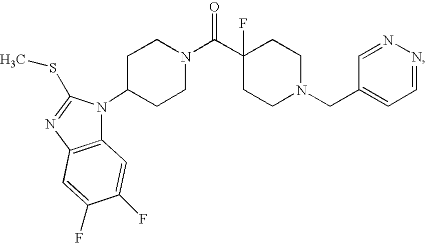

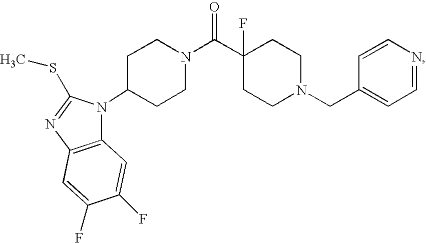

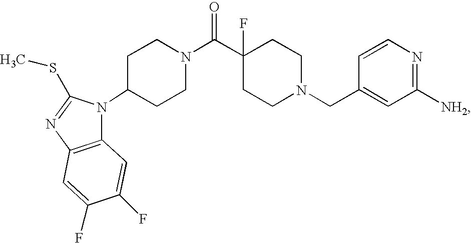

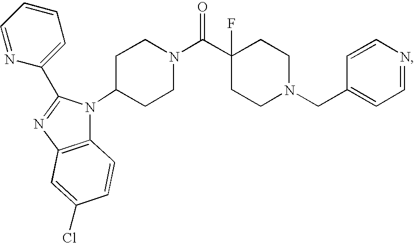

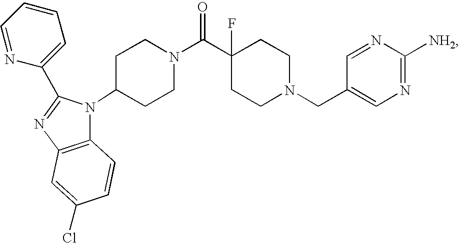

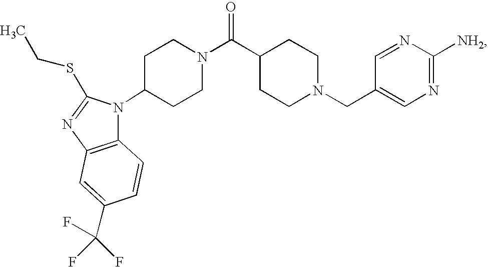

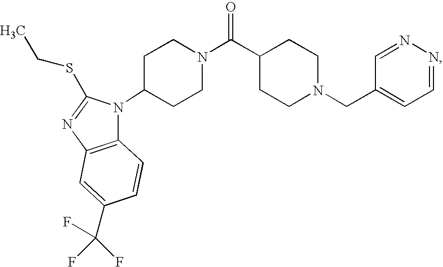

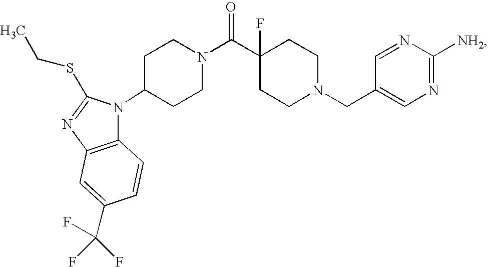

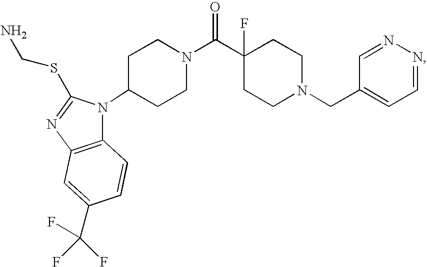

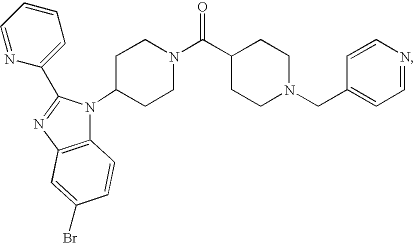

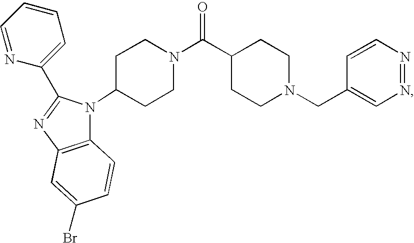

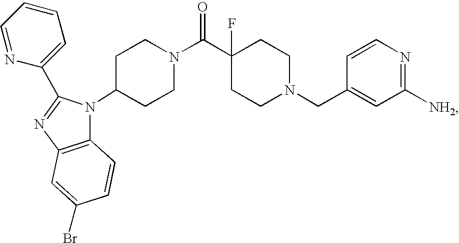

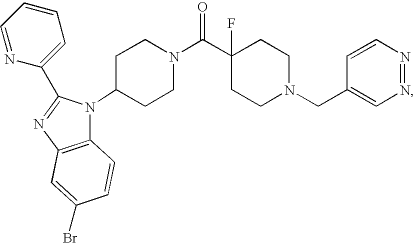

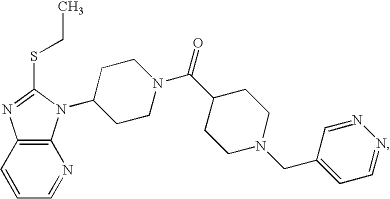

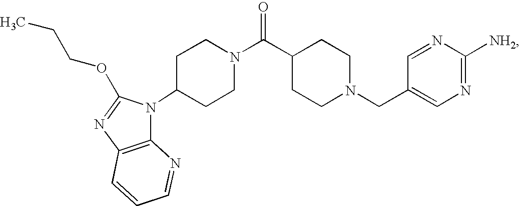

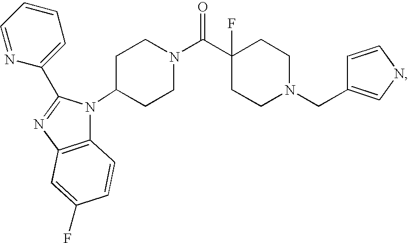

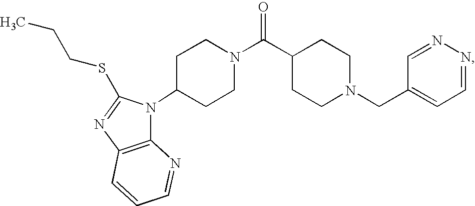

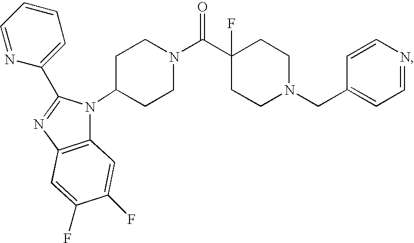

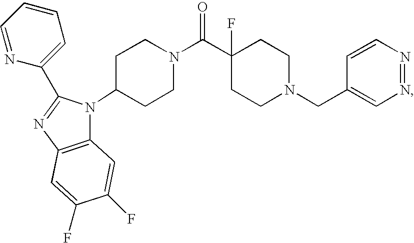

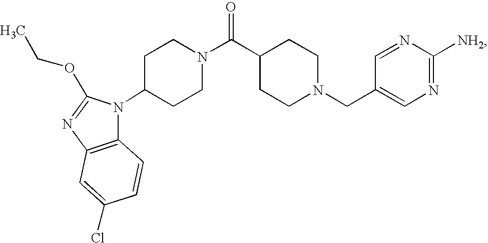

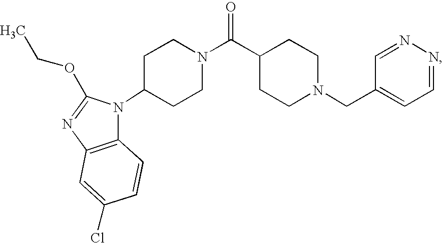

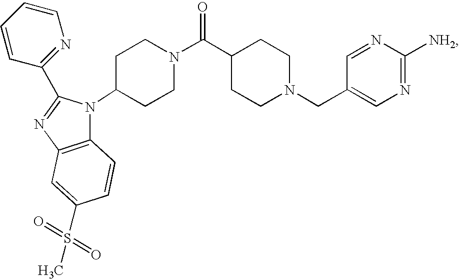

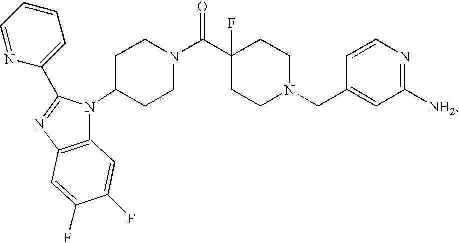

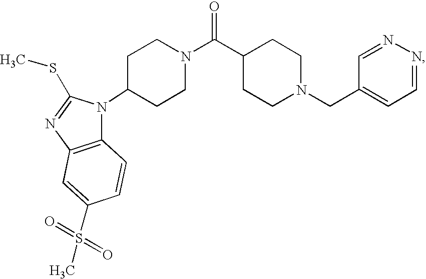

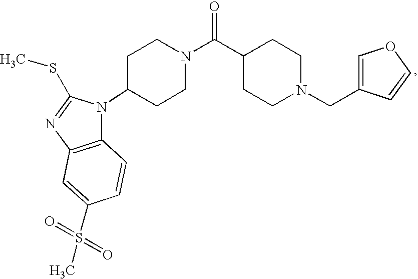

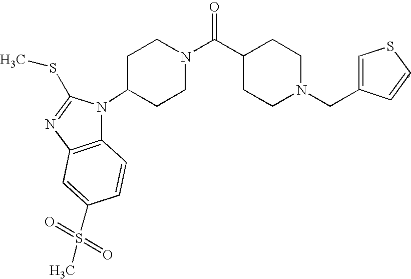

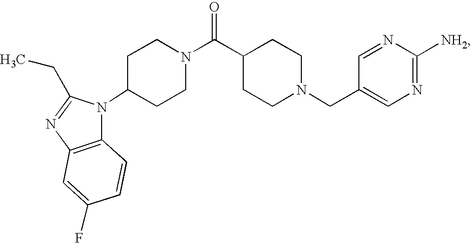

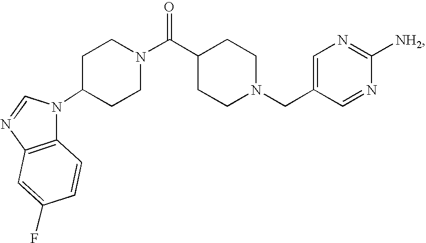

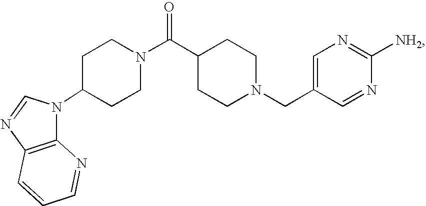

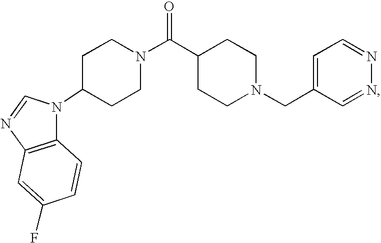

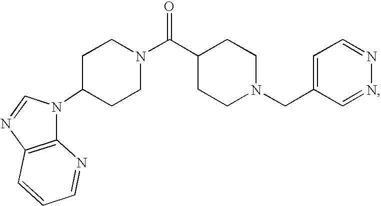

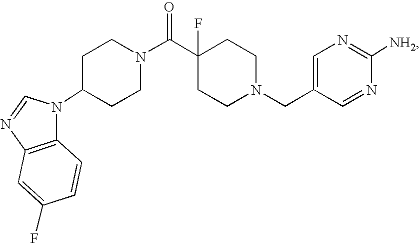

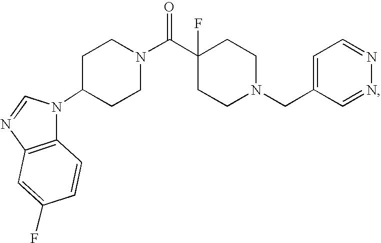

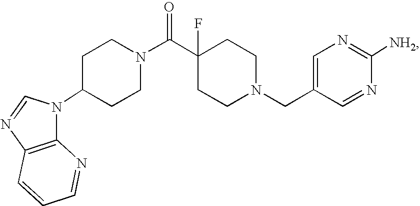

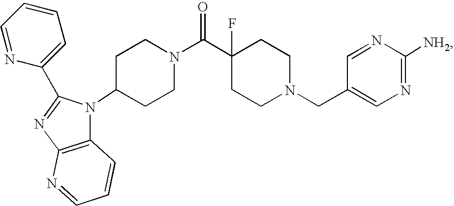

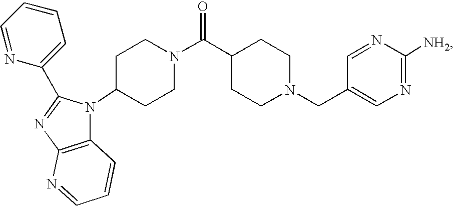

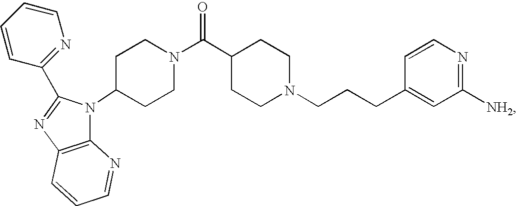

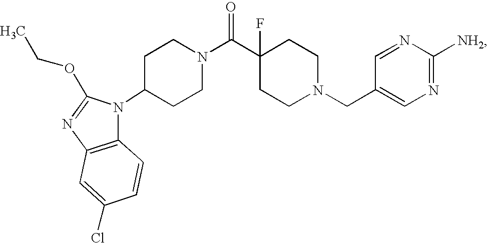

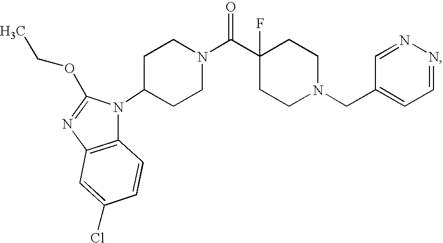

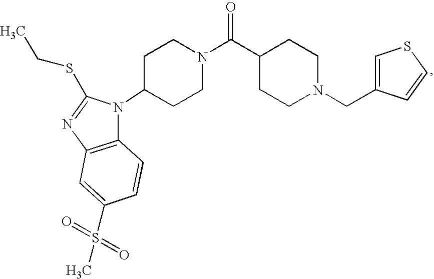

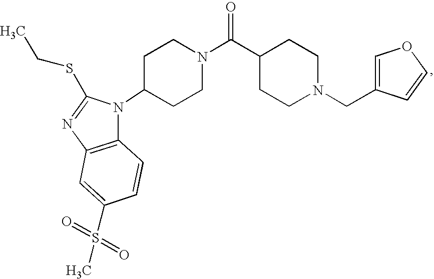

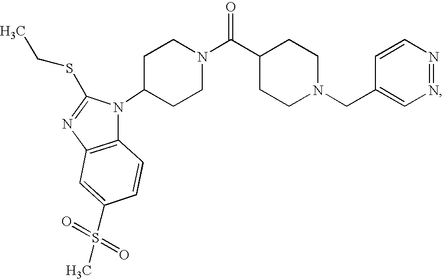

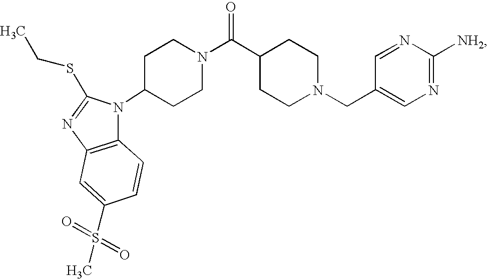

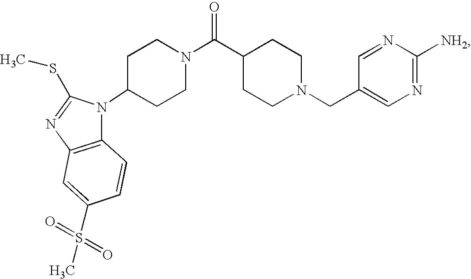

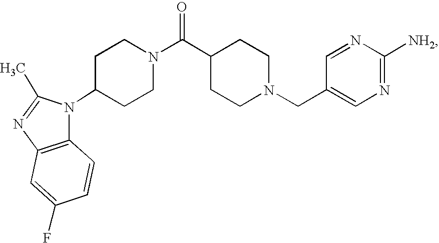

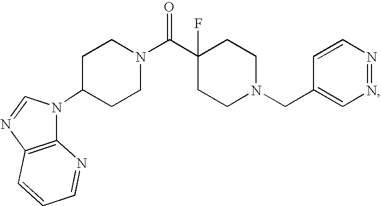

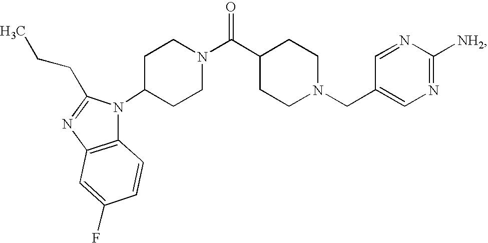

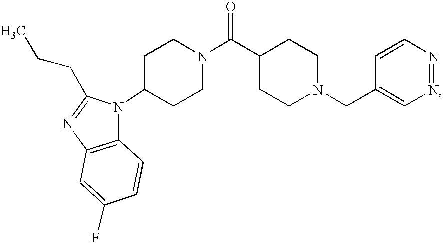

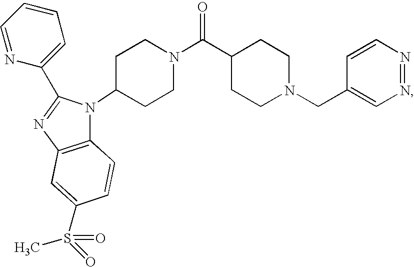

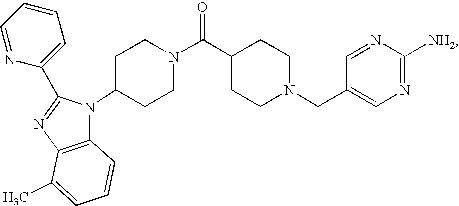

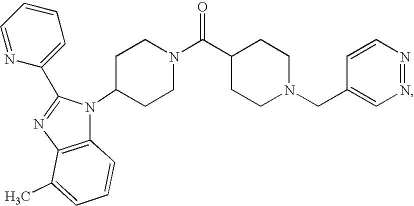

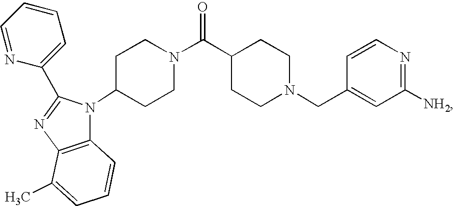

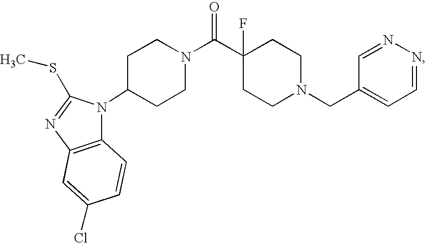

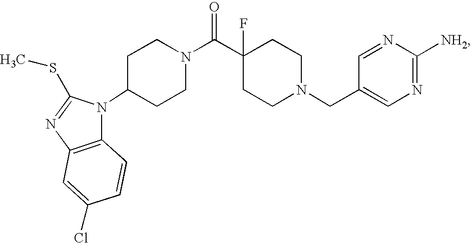

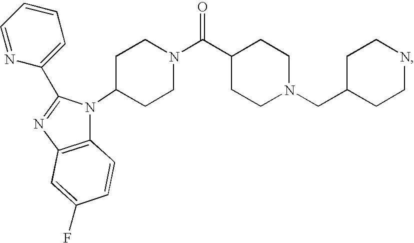

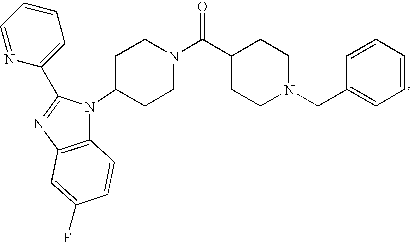

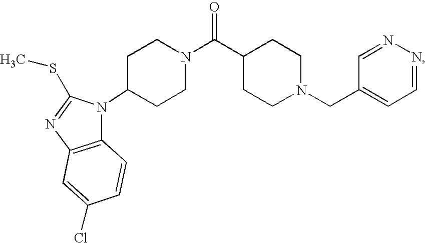

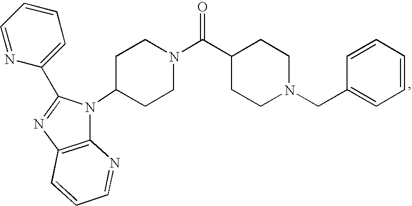

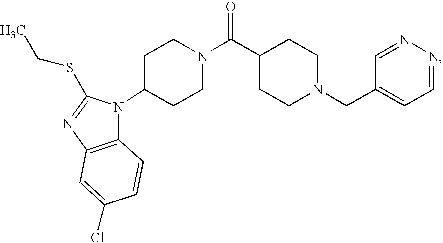

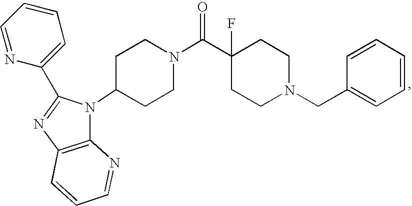

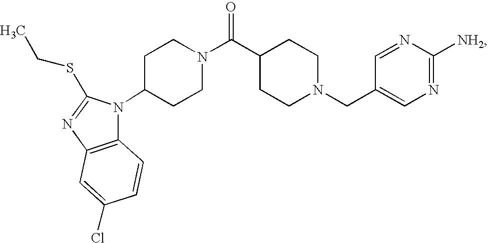

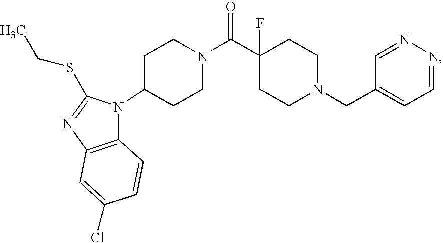

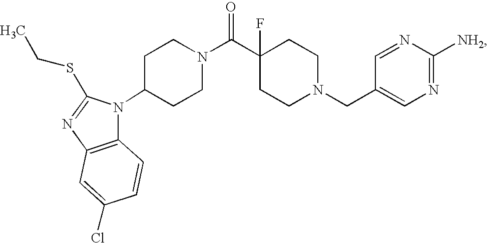

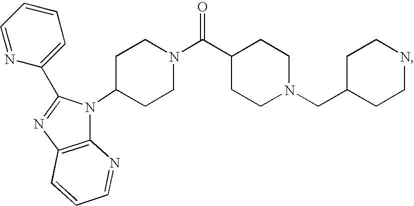

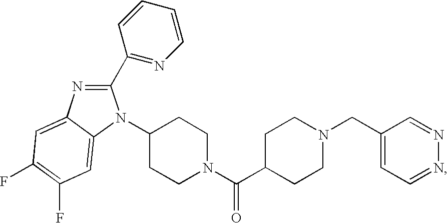

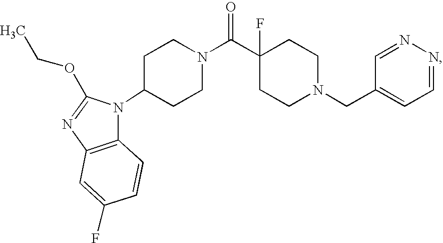

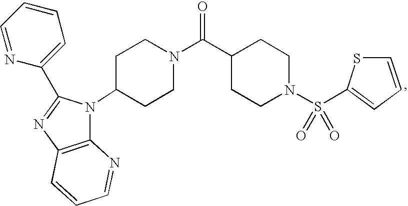

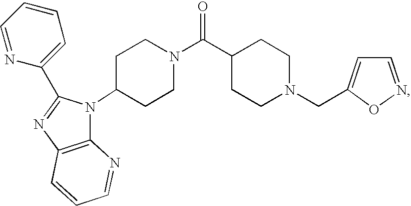

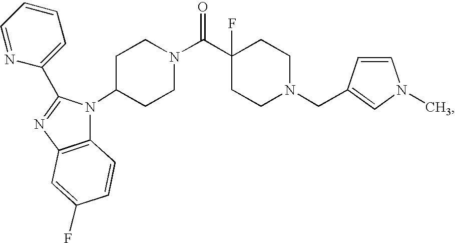

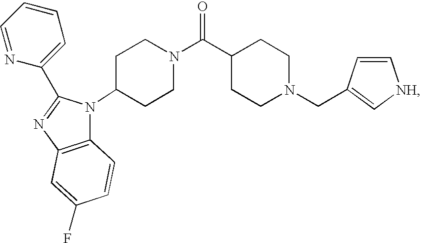

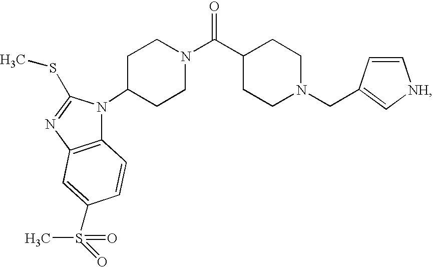

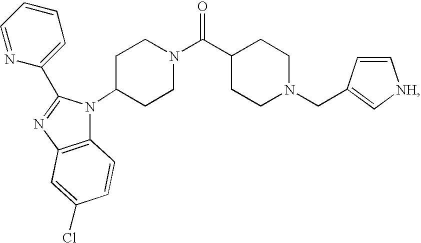

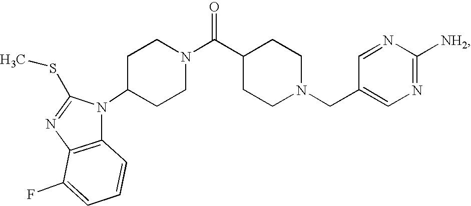

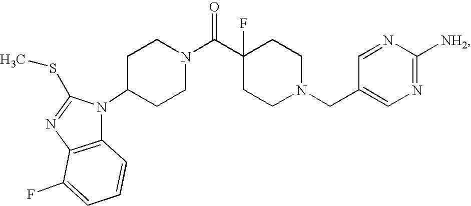

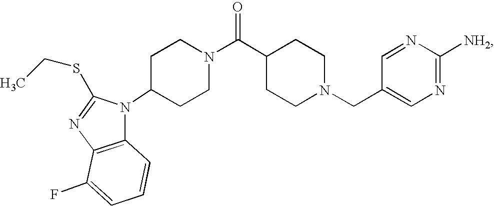

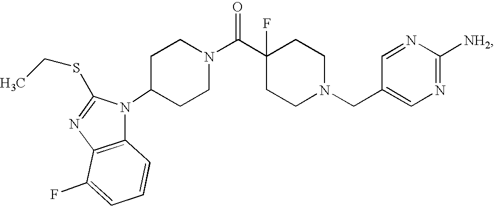

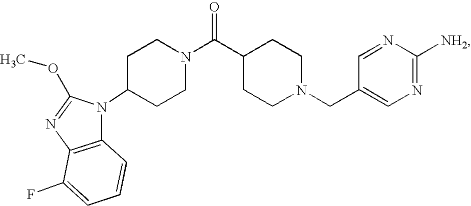

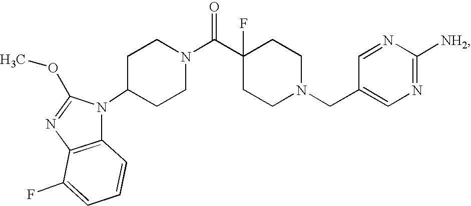

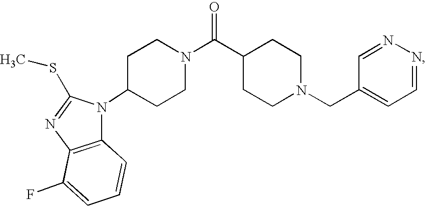

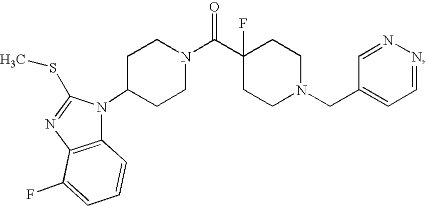

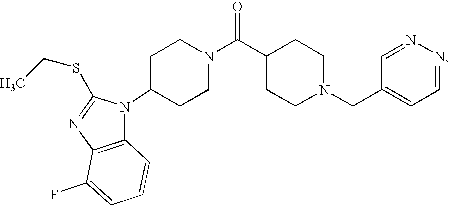

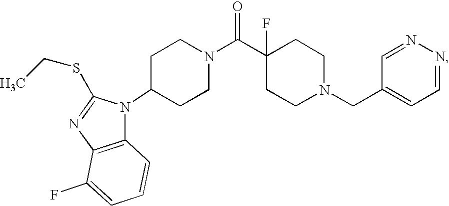

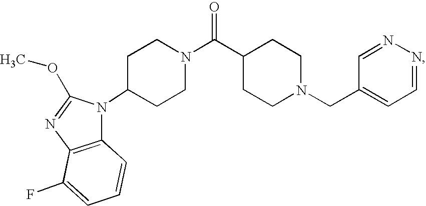

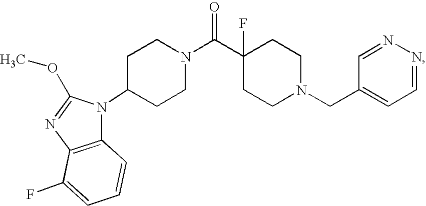

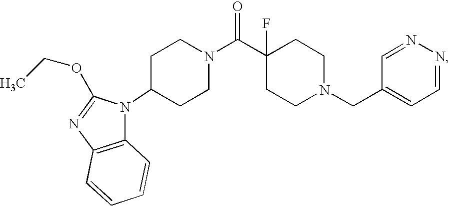

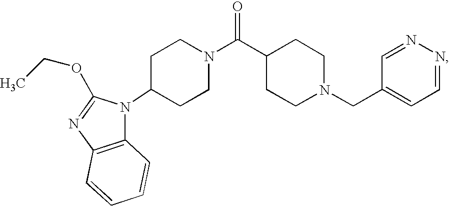

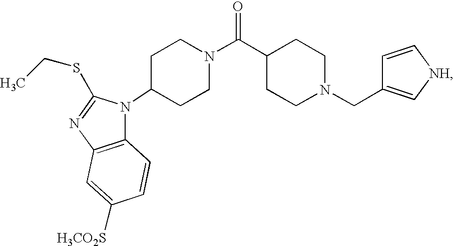

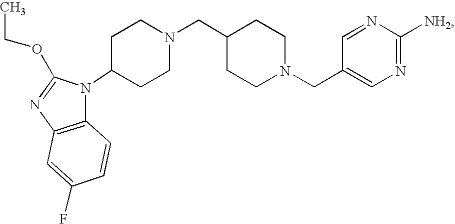

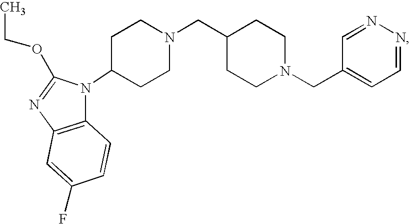

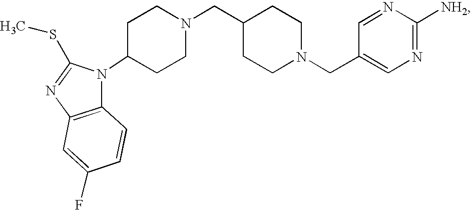

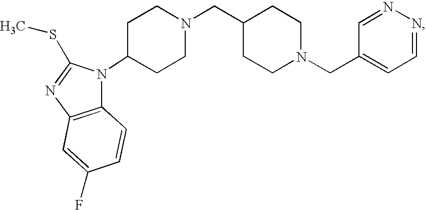

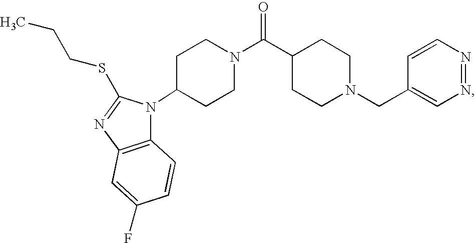

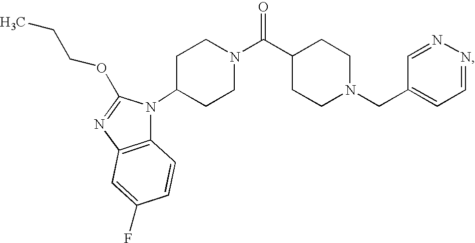

and

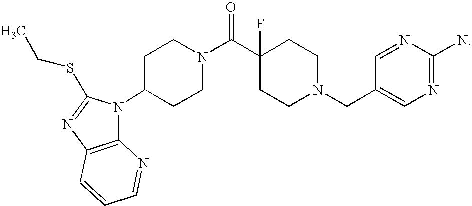

In another embodiment, the H₃ antagonists/inverse agonists of the present invention can have the following structure: 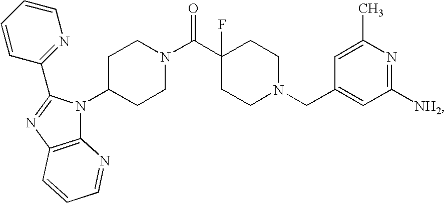
as described in U.S. Provisional Application No. 60/718,673, filed Sep. 20, 2005, and which is herein incorporated by reference in its entirety.

In another embodiment, the H₃ antagonists/inverse agonists of the present invention can have the following Formula (VII): 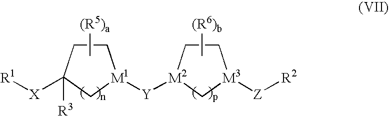
as described in U.S. Provisional Application No. 60/692,110, filed Jun. 20, 2005, and which is herein incorporated by reference in its entirety. Non-limiting examples of compounds of Formula (VII) include:

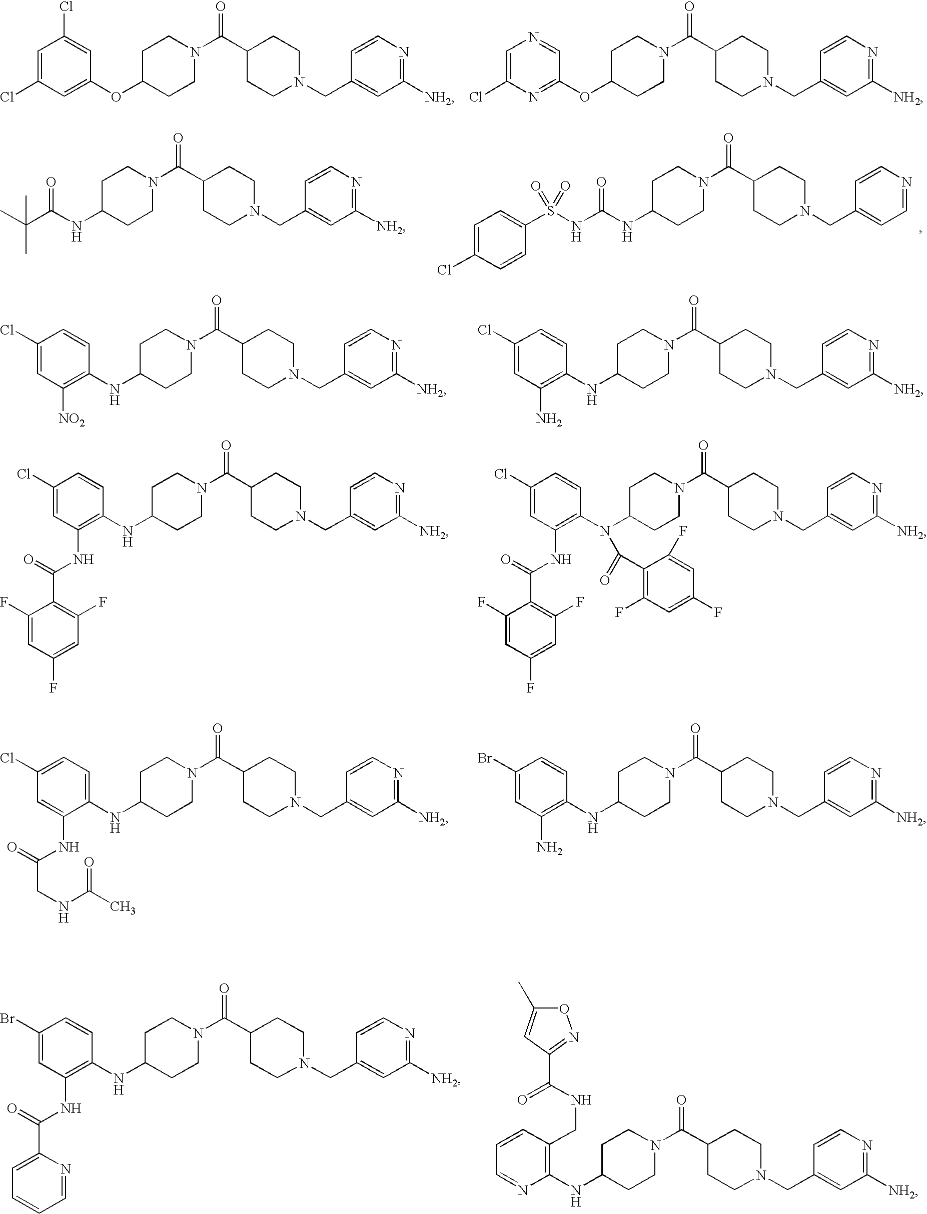

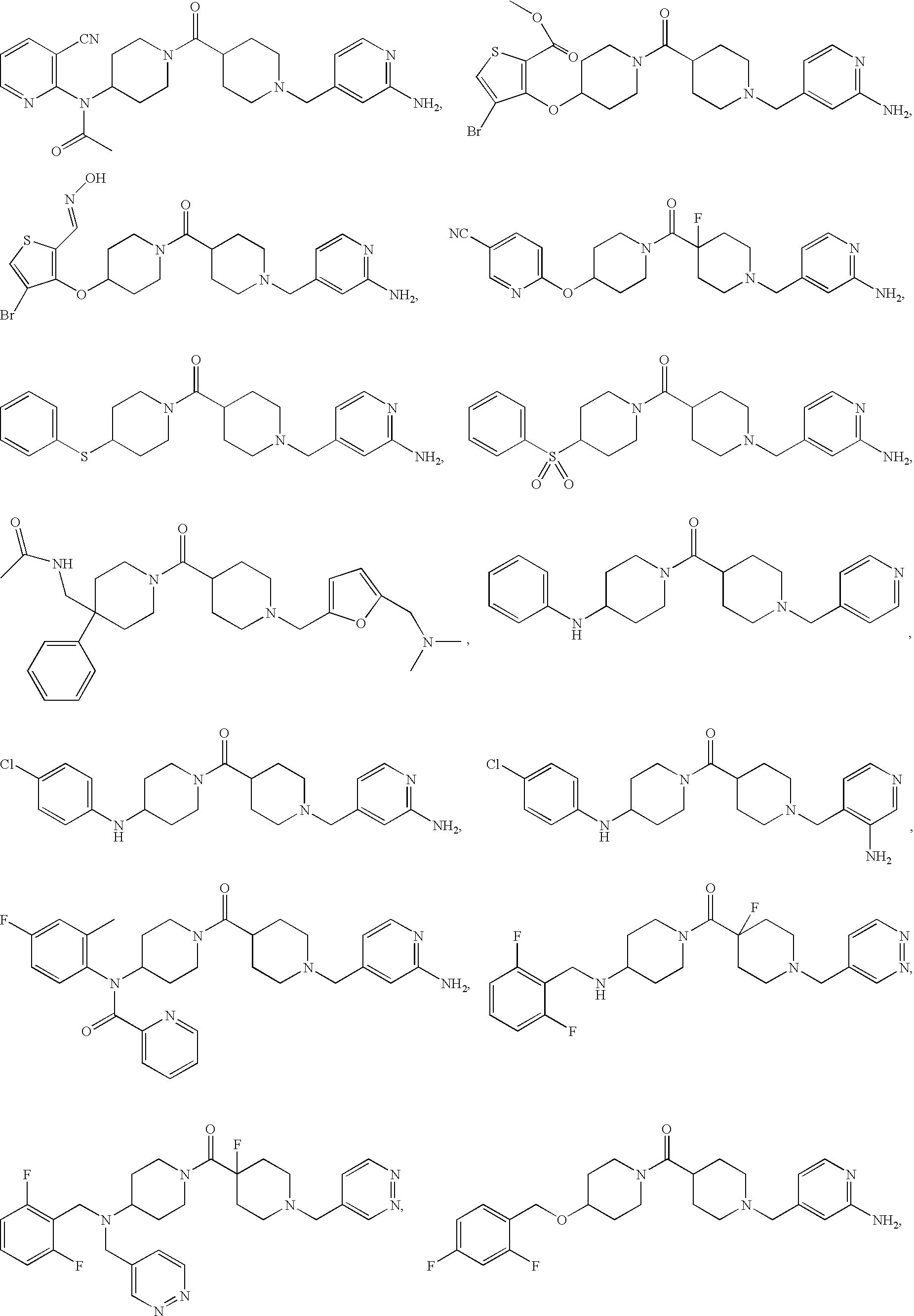

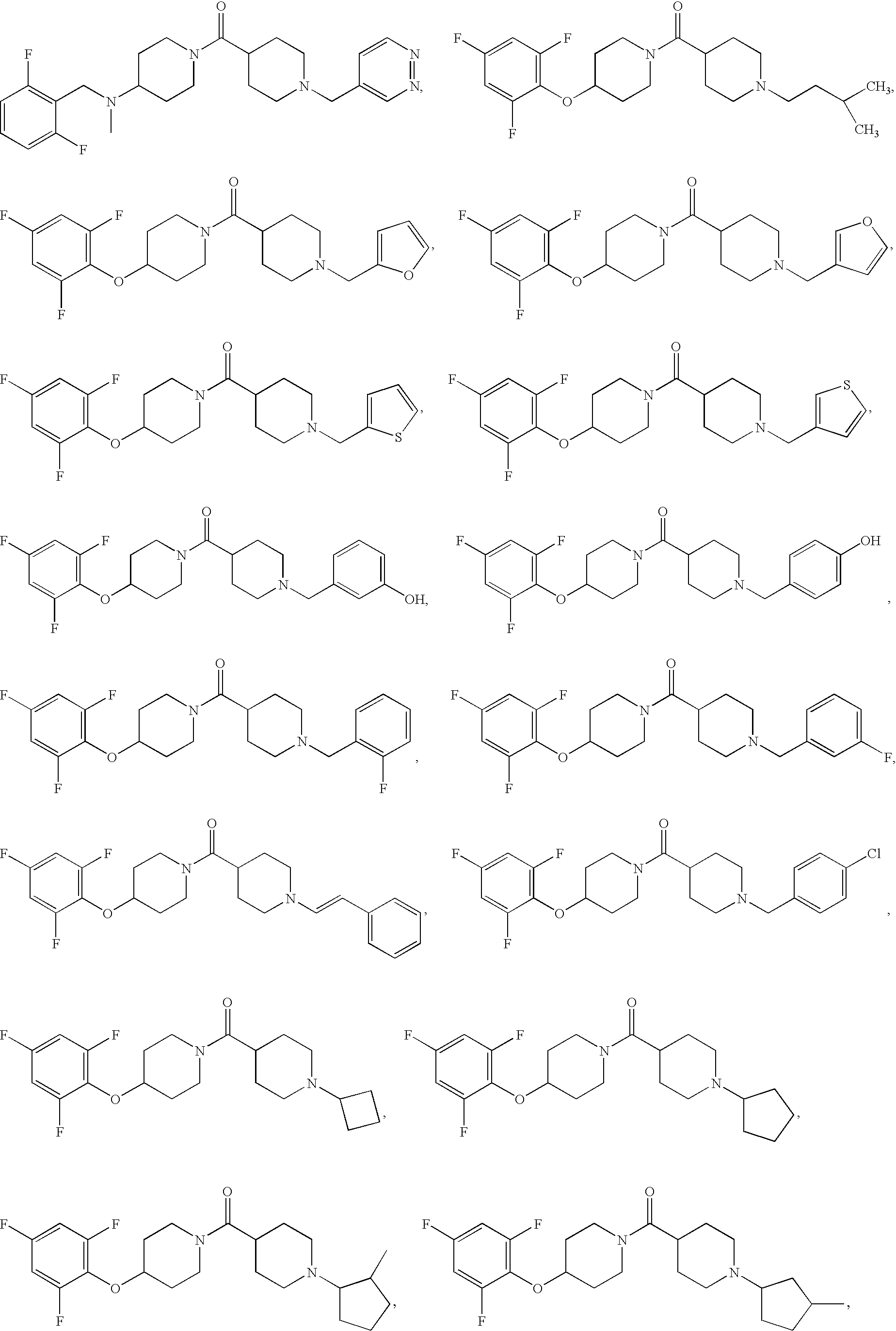

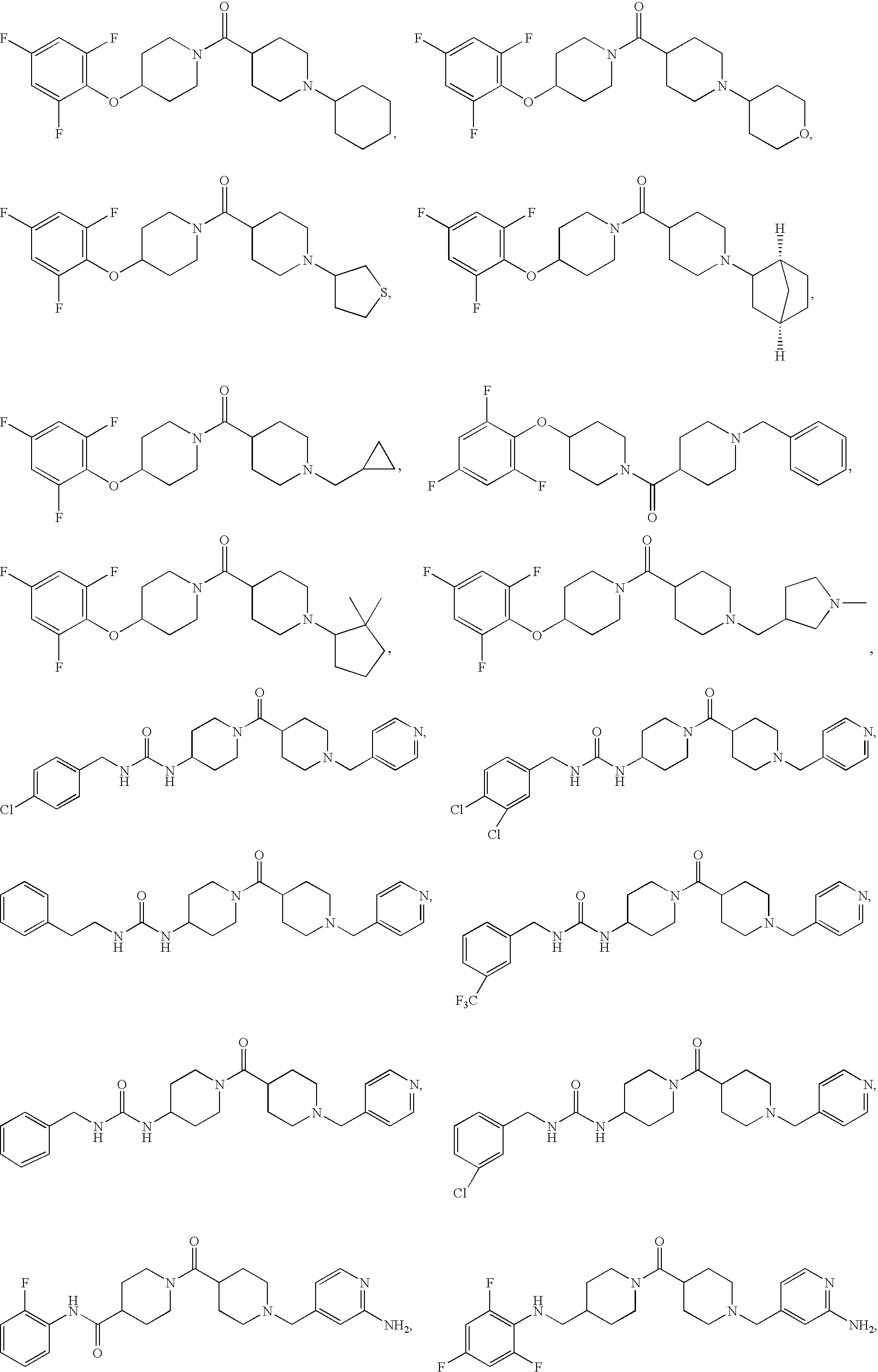

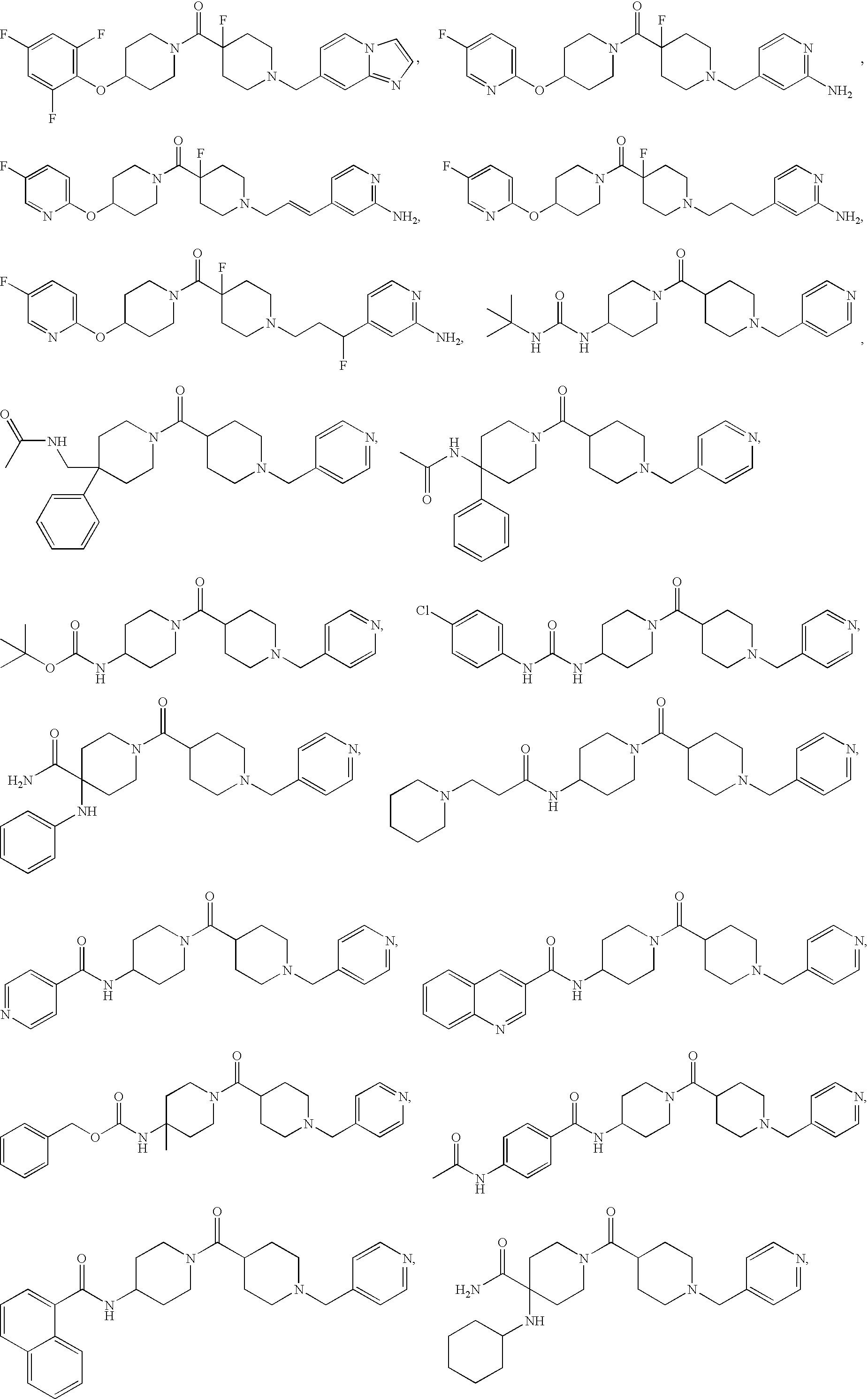

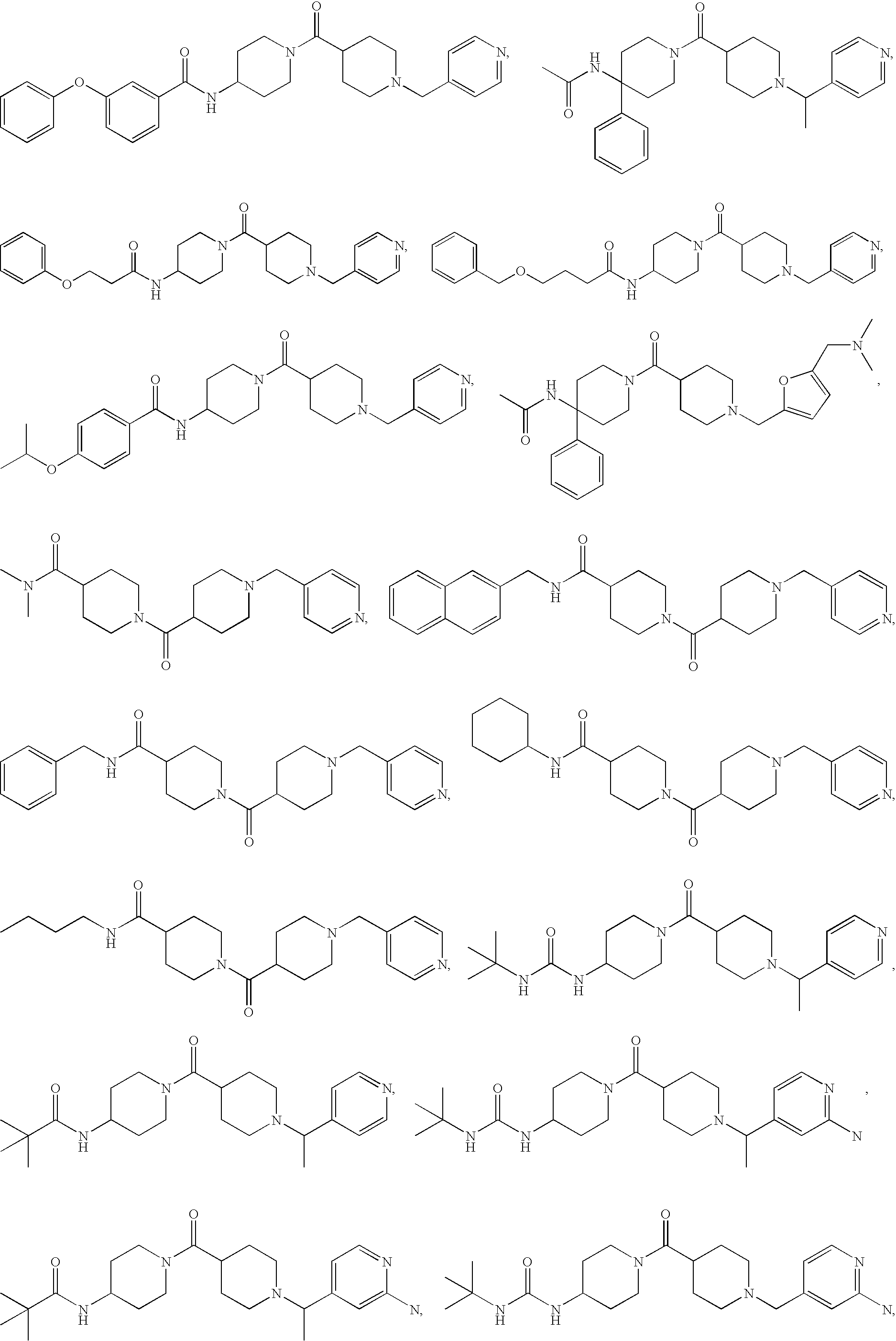

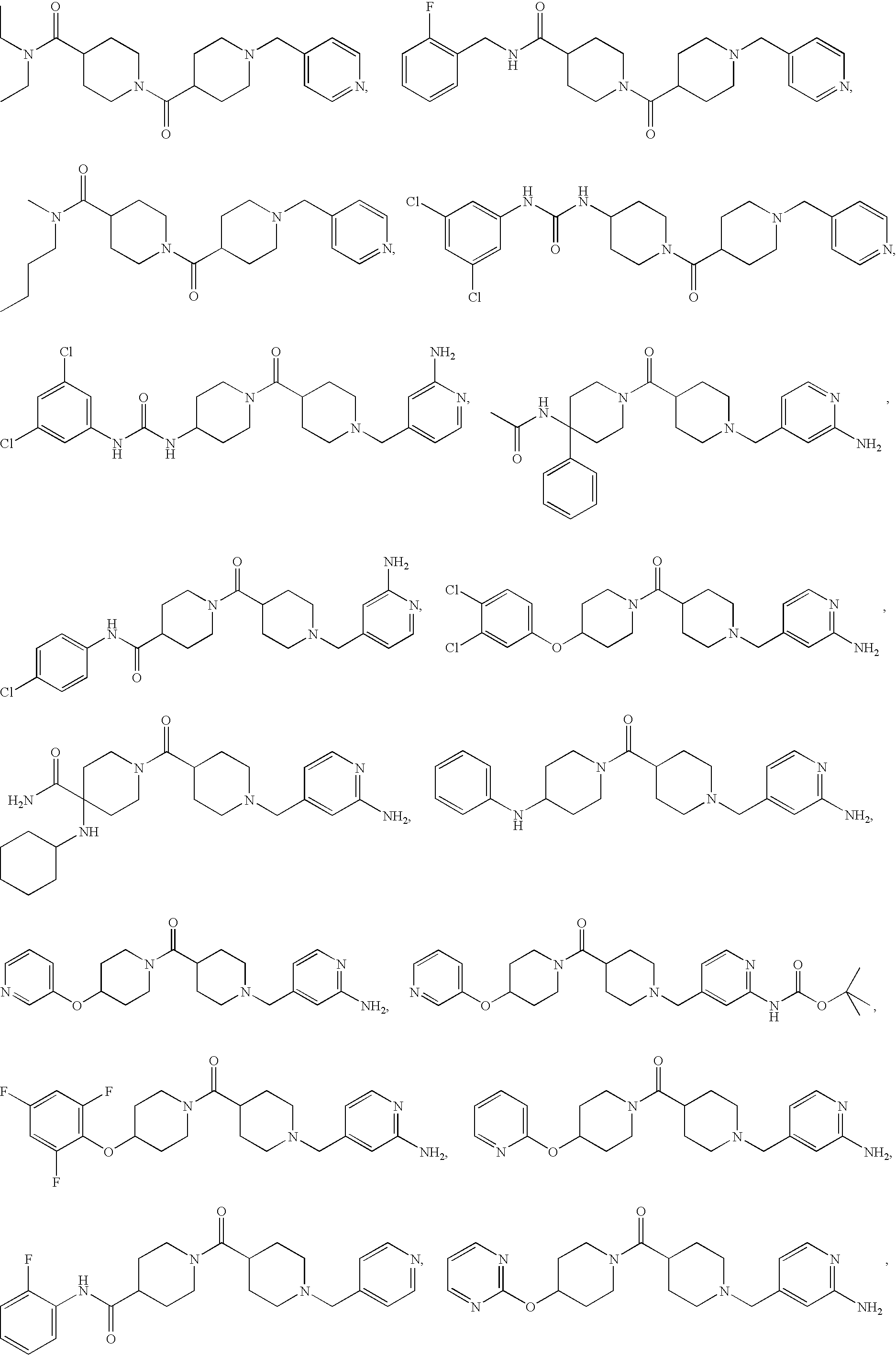

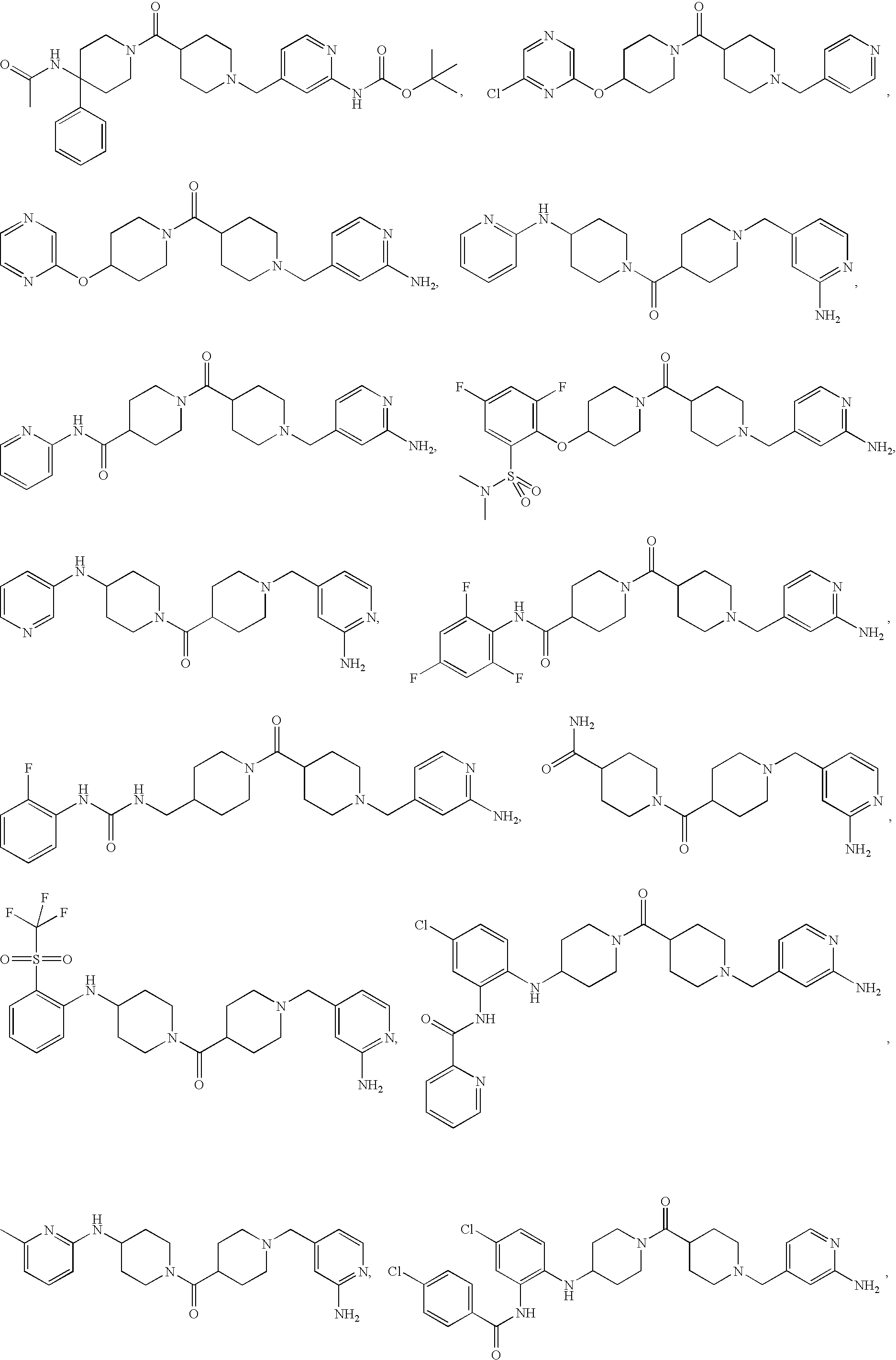

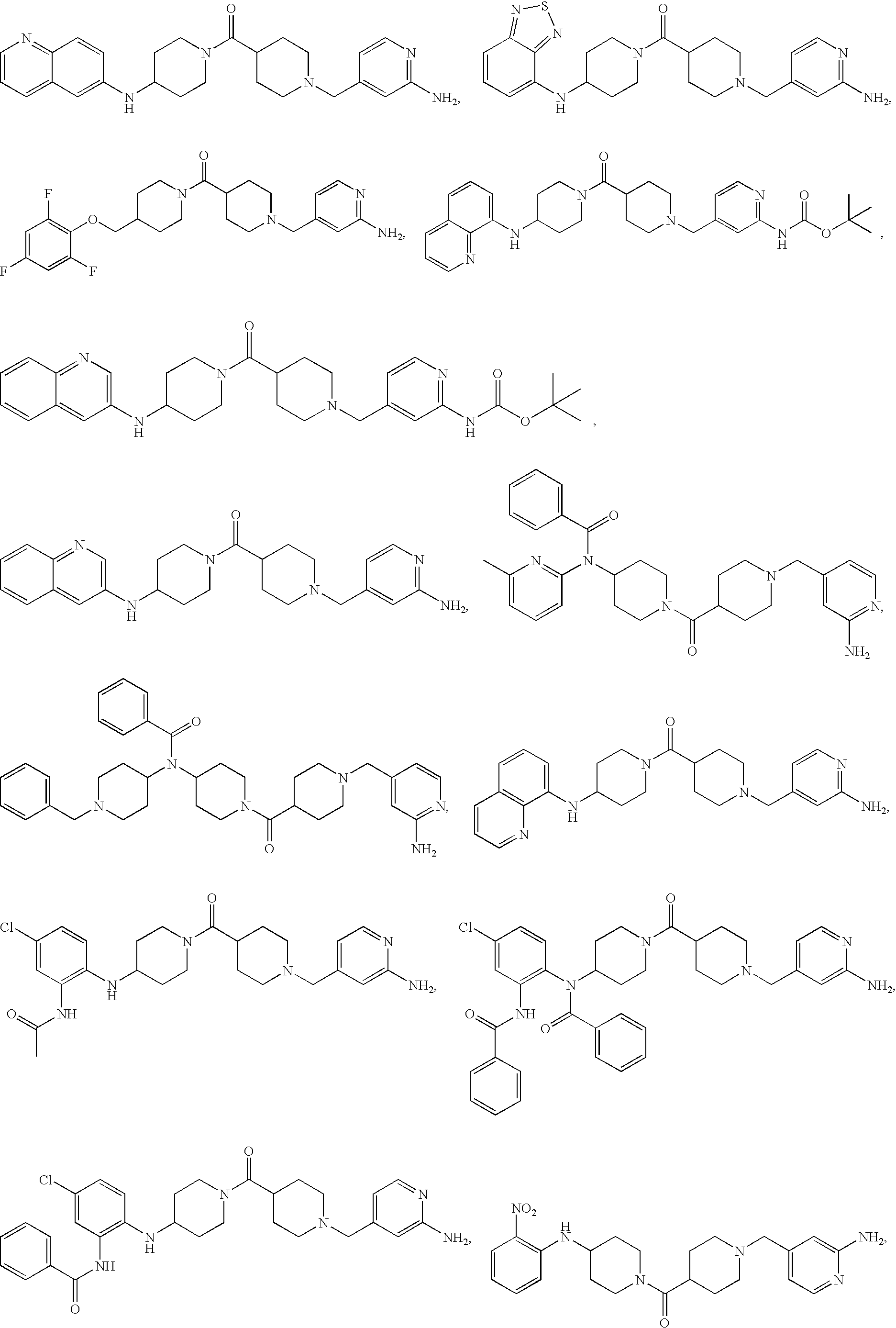

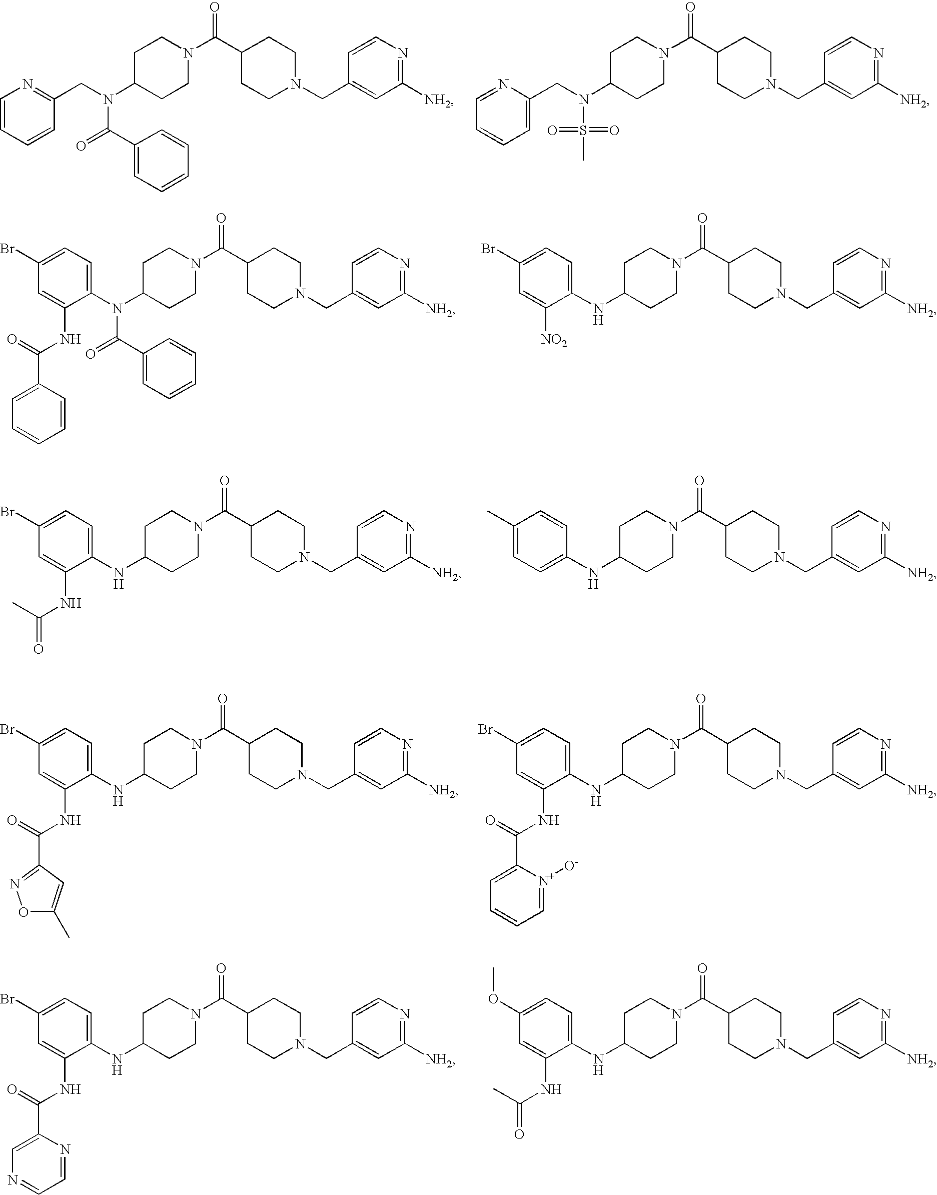

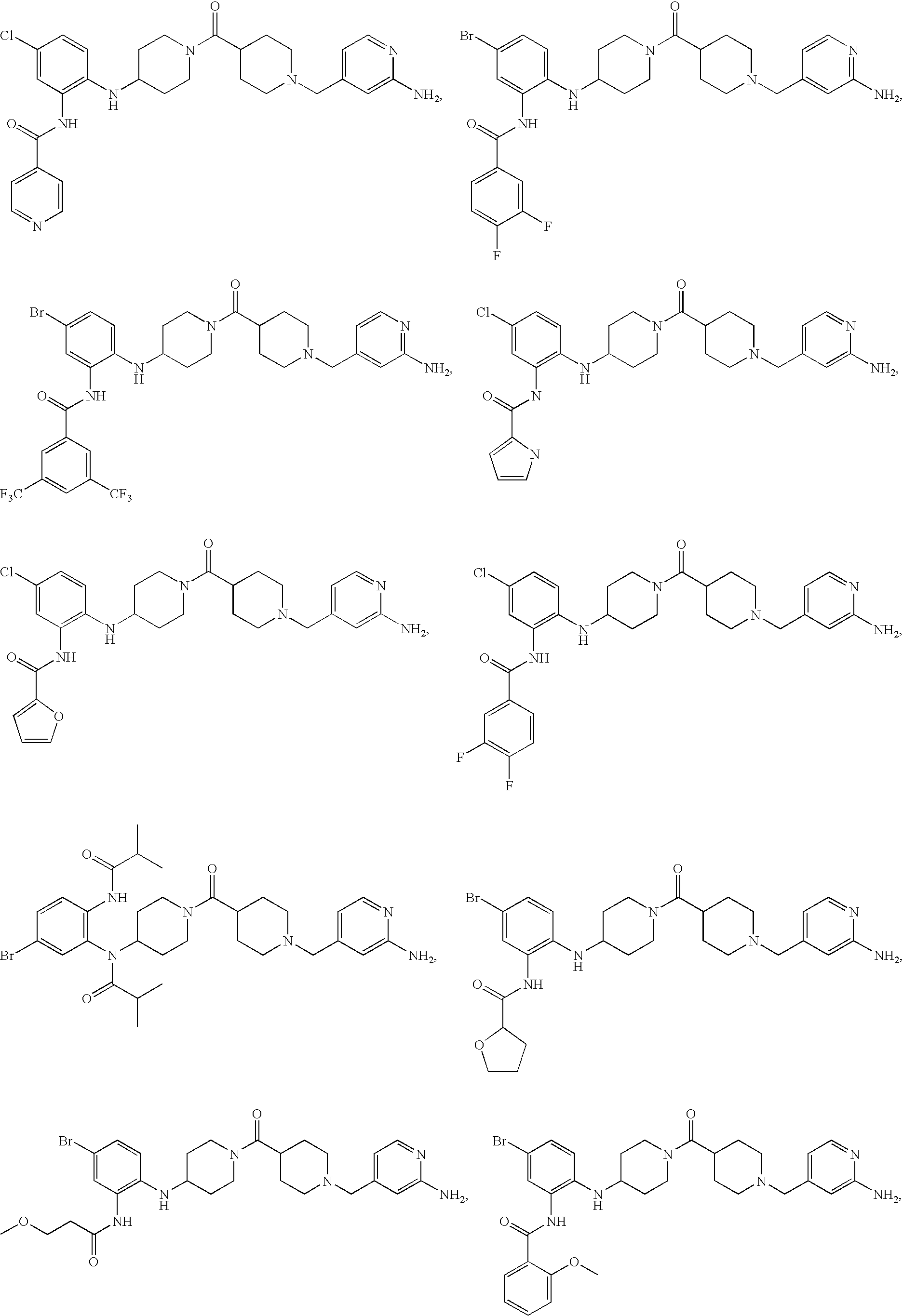

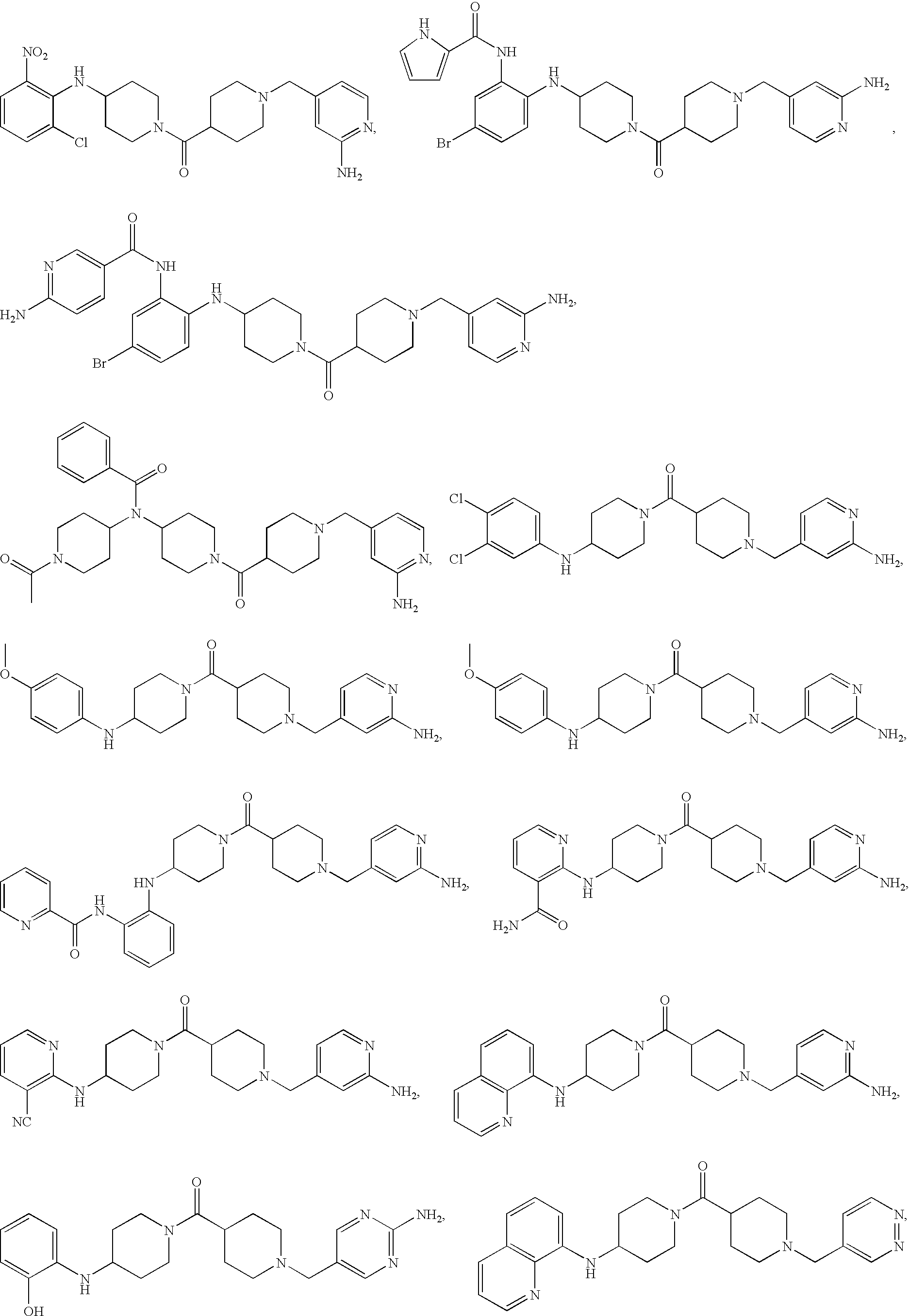

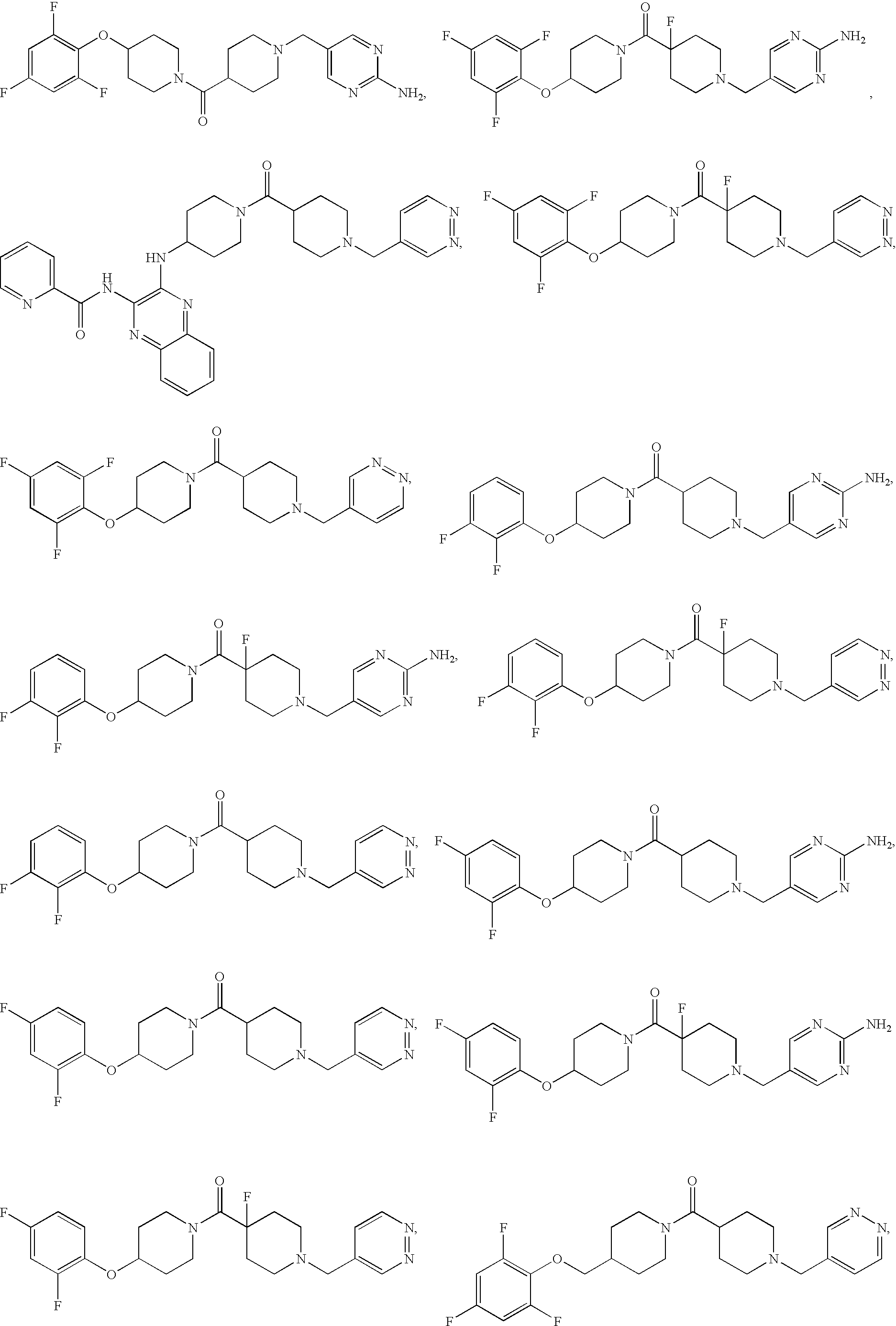

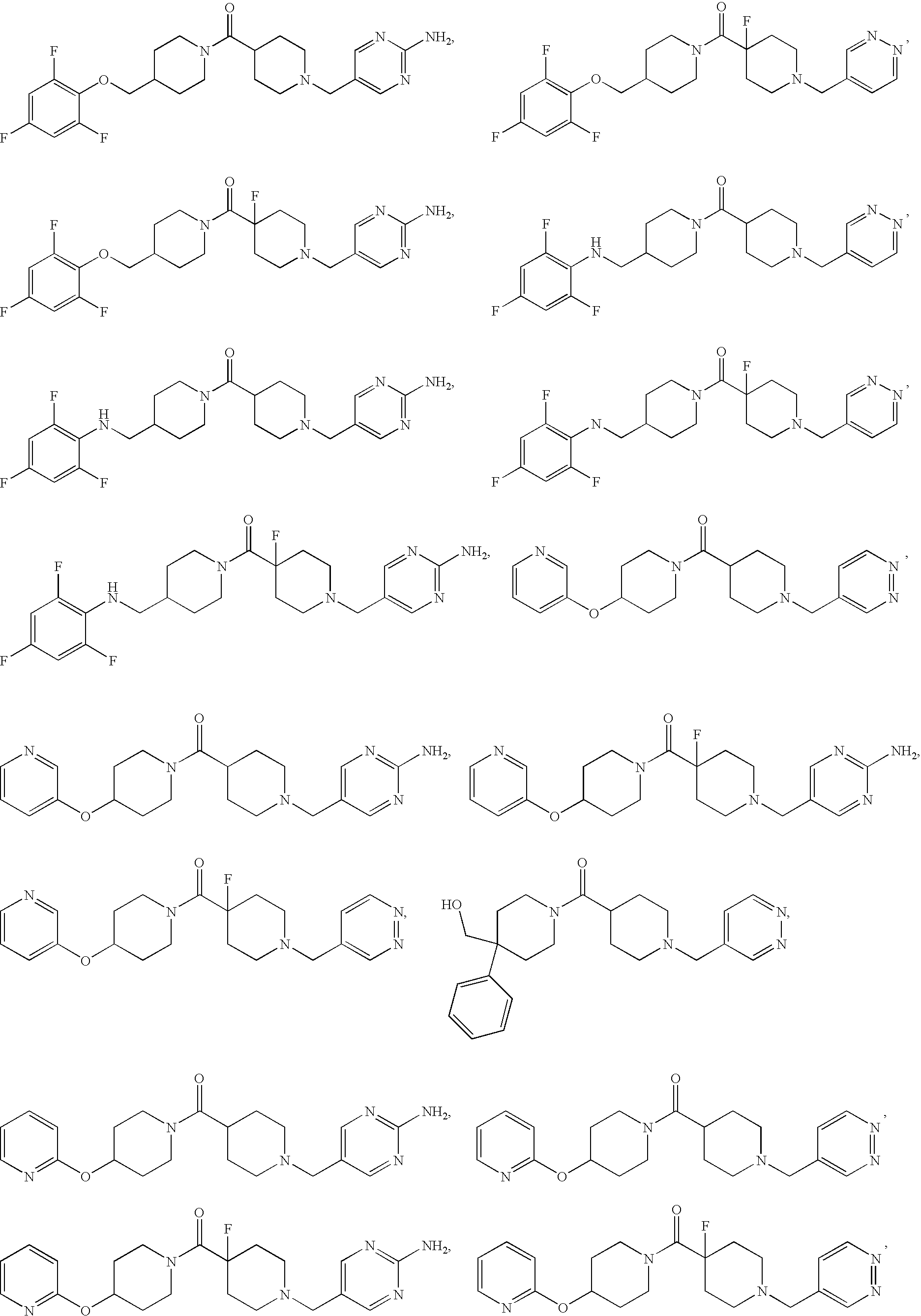

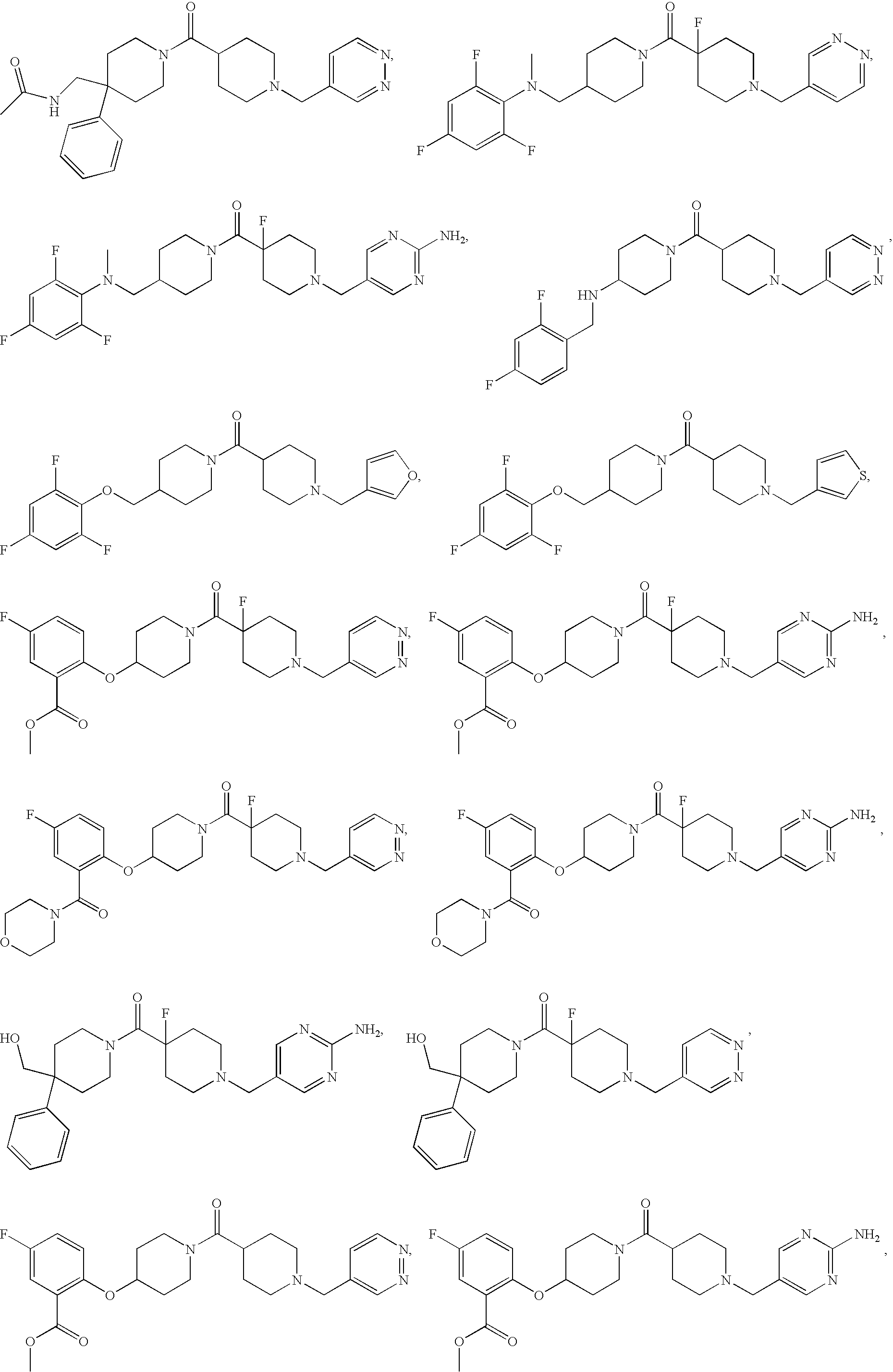

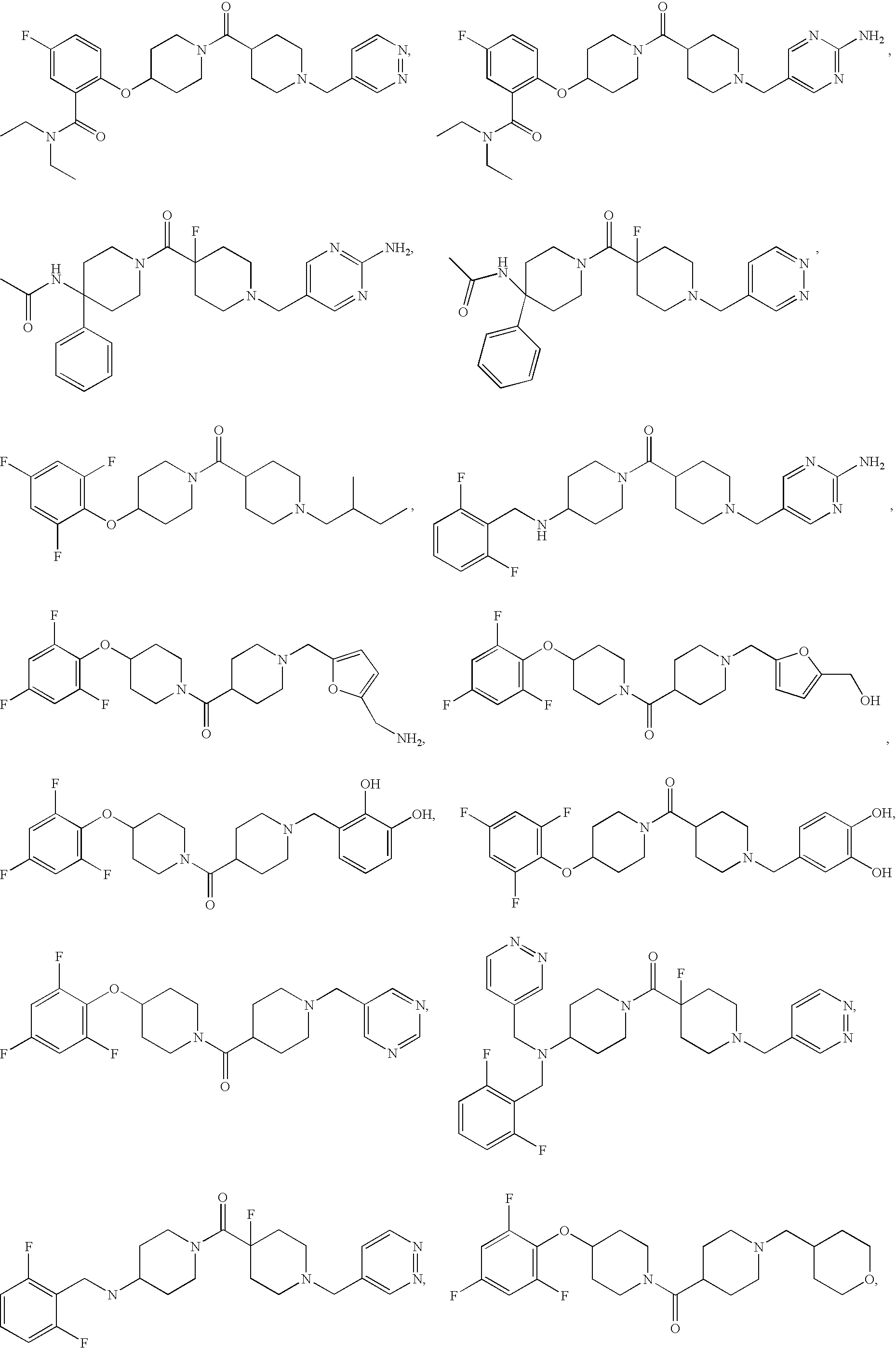

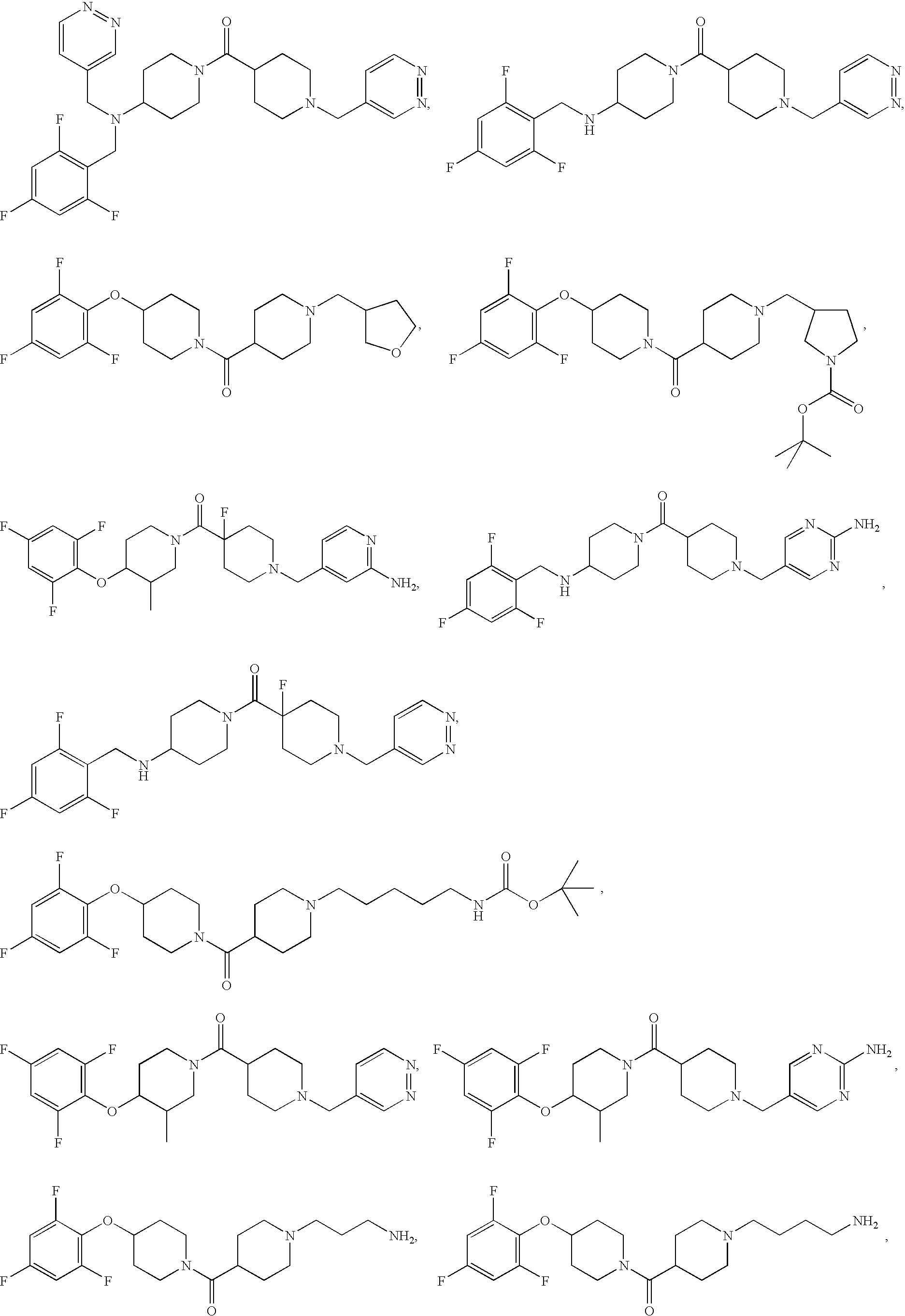

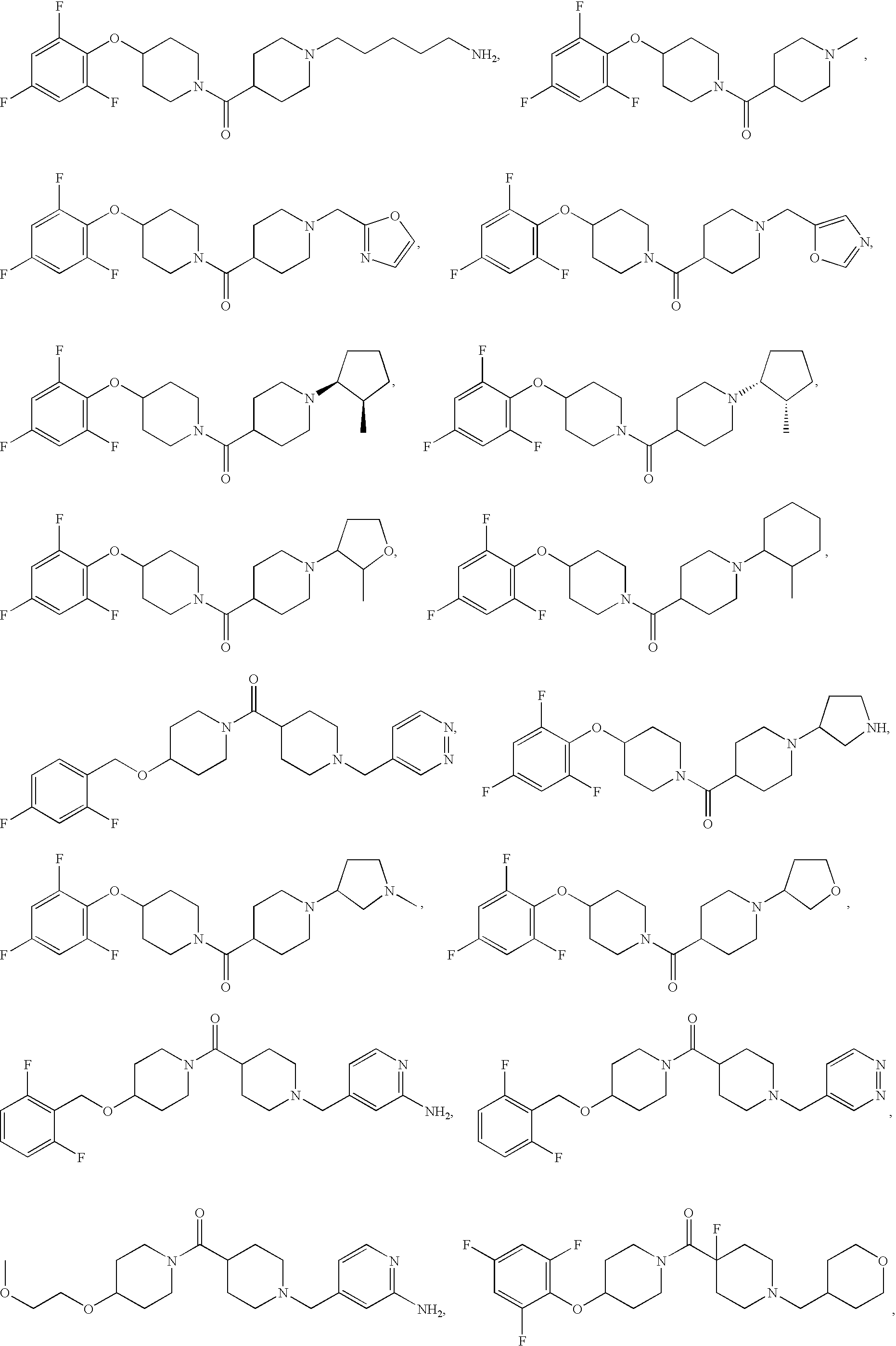

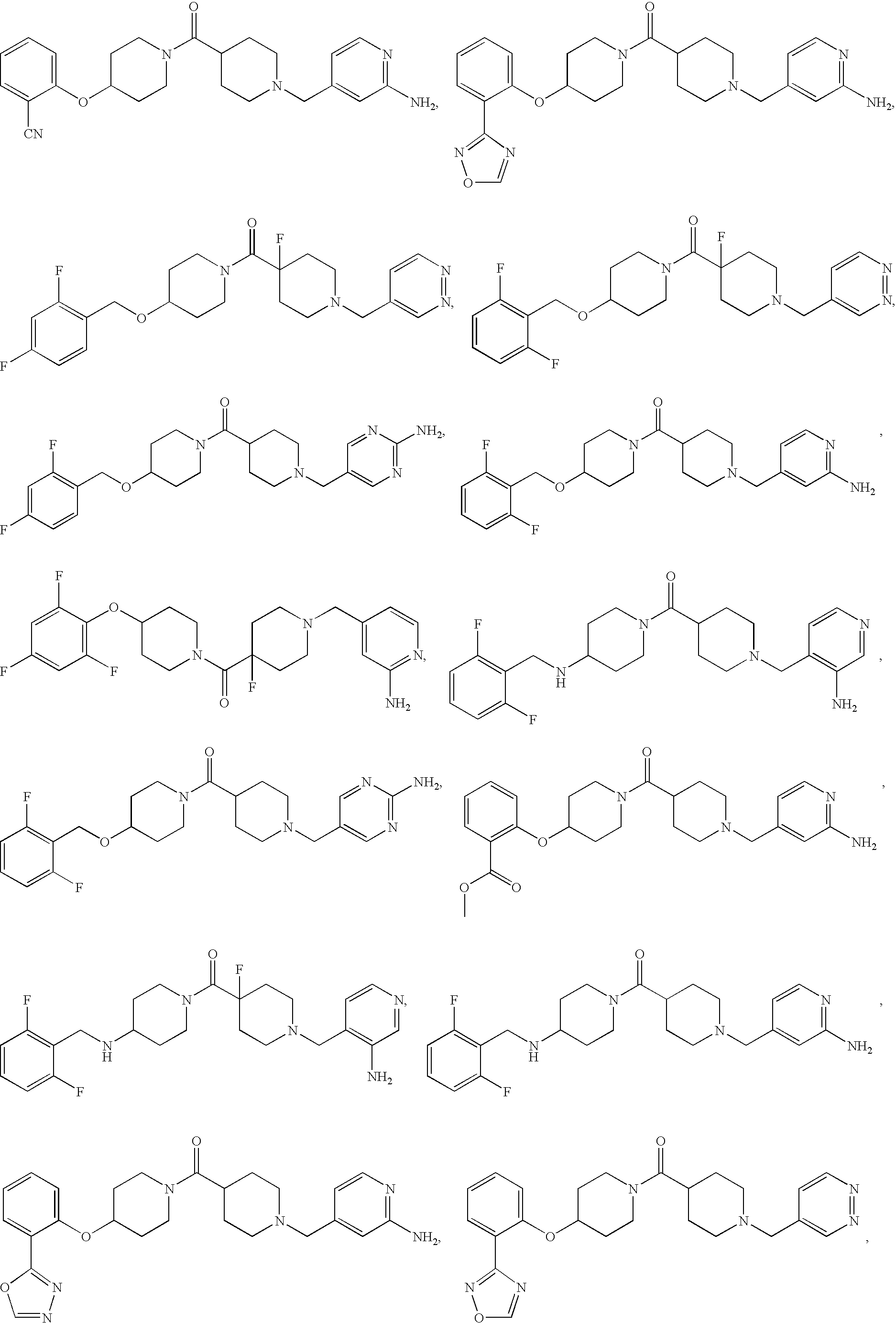

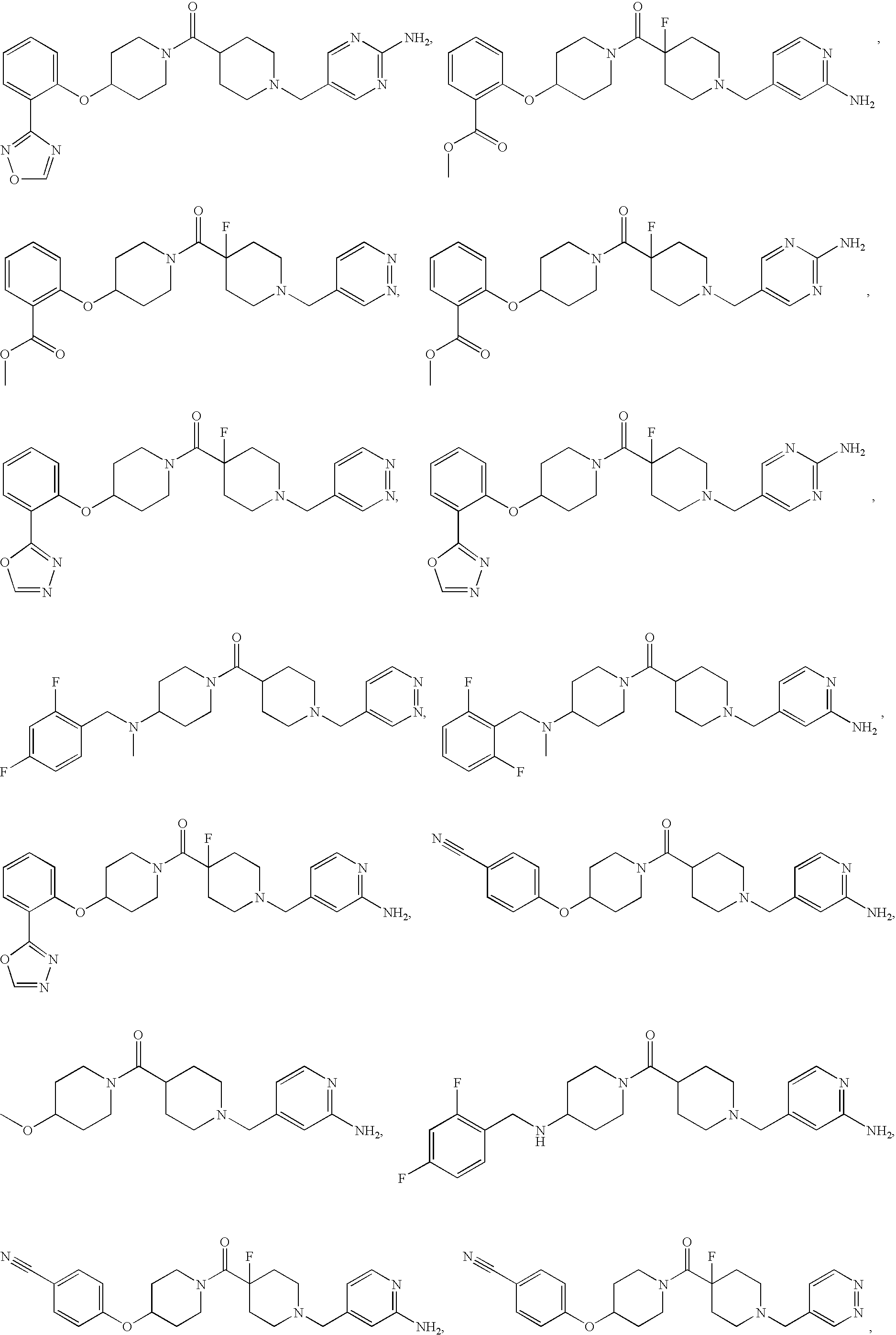

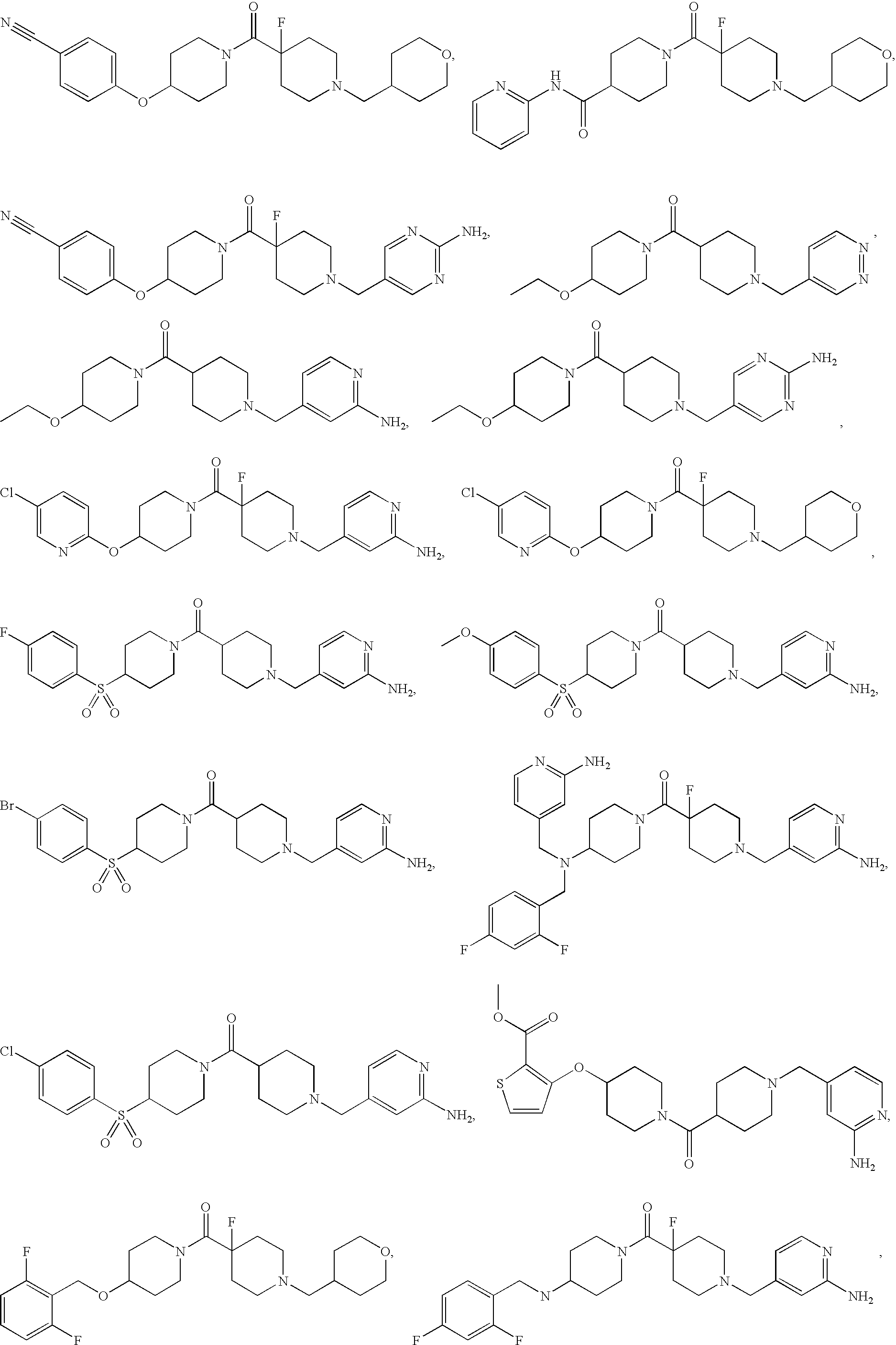

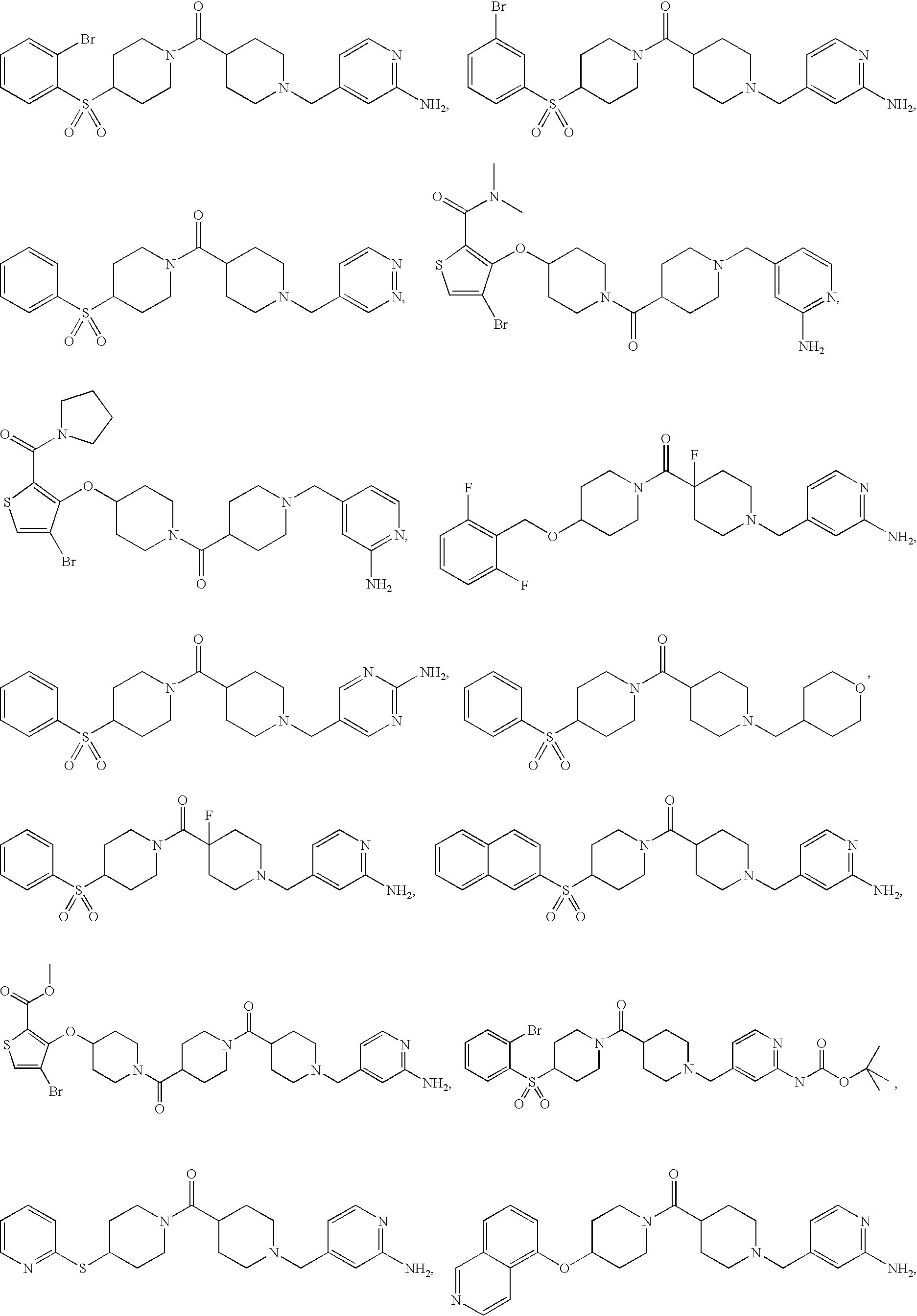

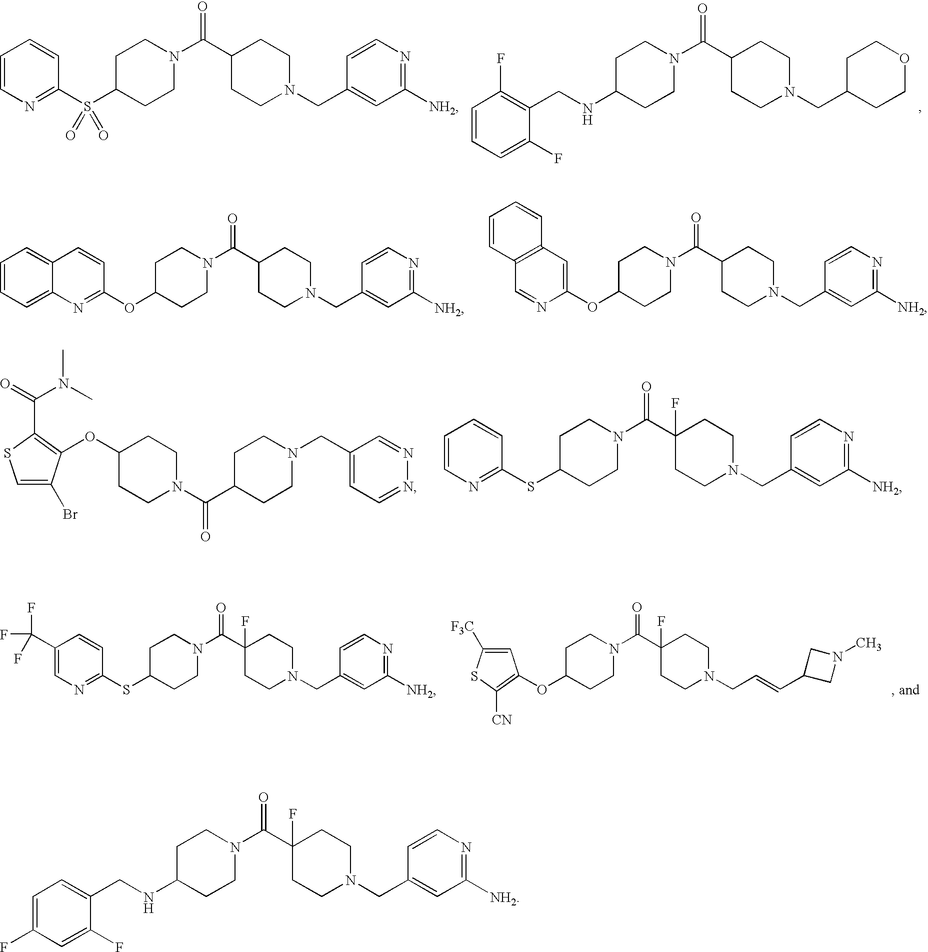

In another embodiment, the H₃ antagonists/inverse agonists of the present invention can have the following Formula (VIII): 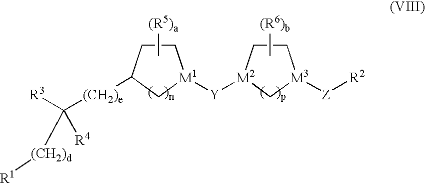
as described in U.S. Provisional Application No. 60/692,175, filed Jun. 20, 2005, and which is herein incorporated by reference in its entirety. Non-limiting examples of compounds of Formula (VIII) include:

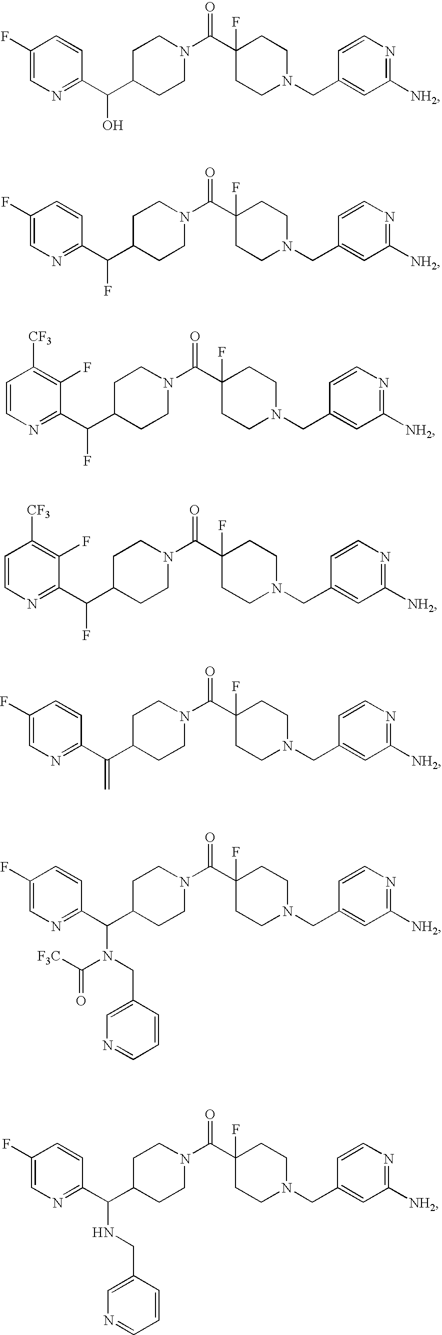

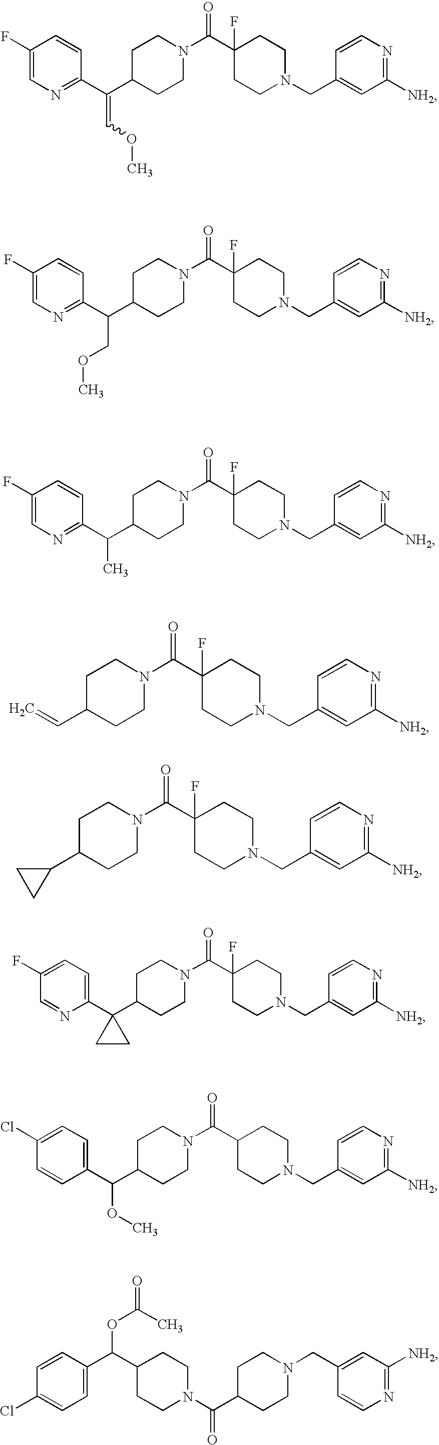

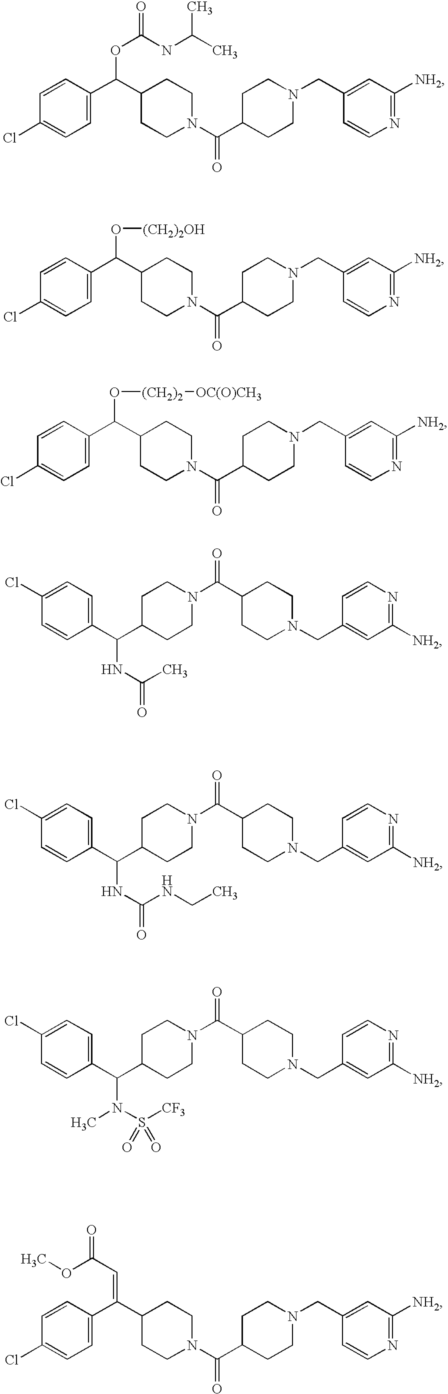

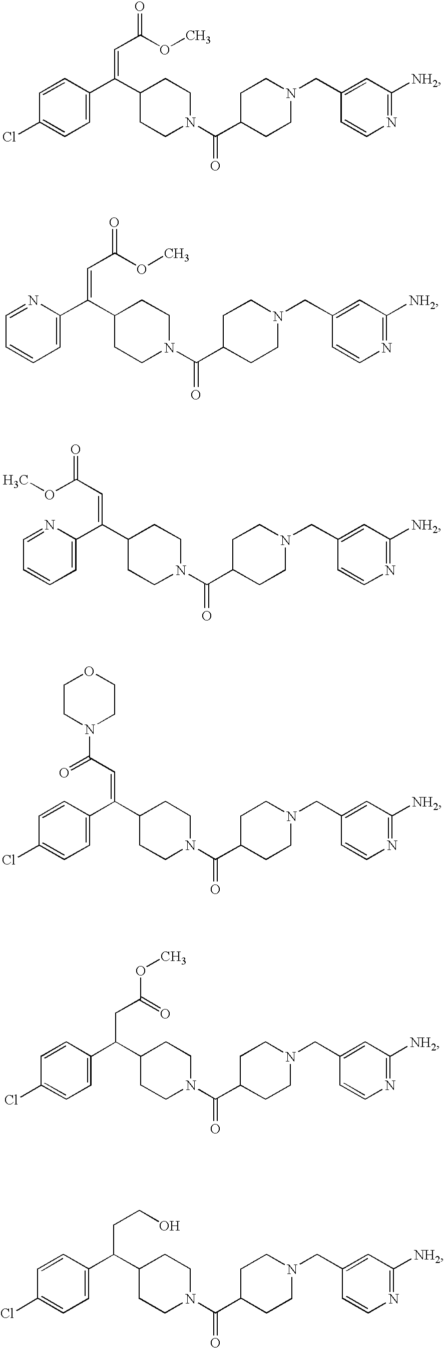

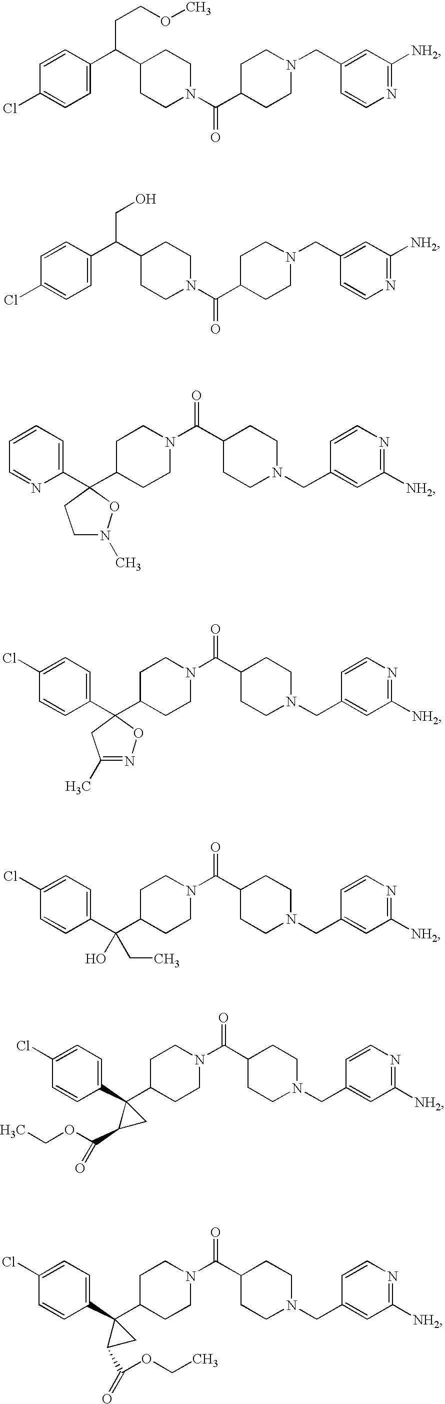

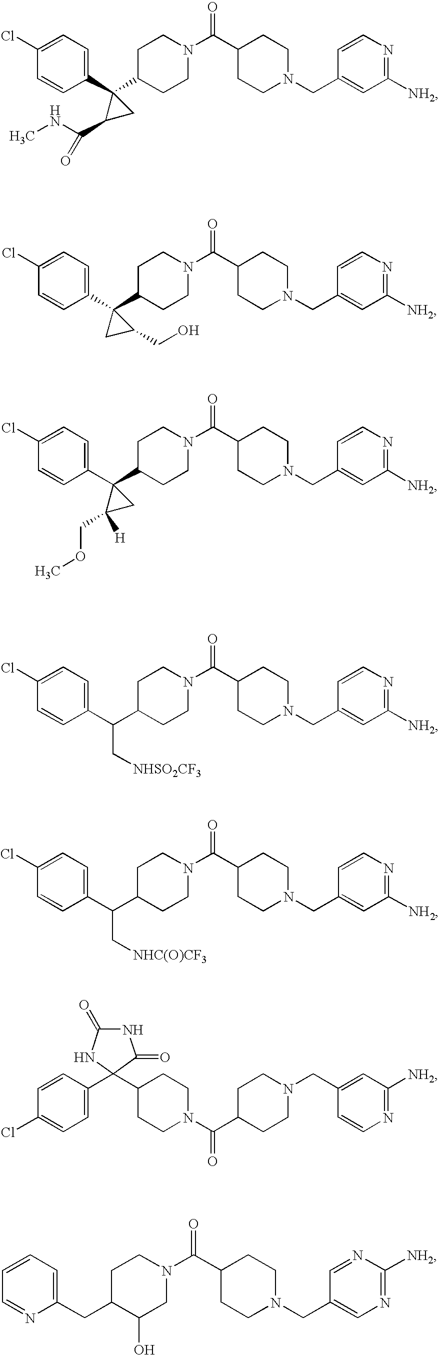

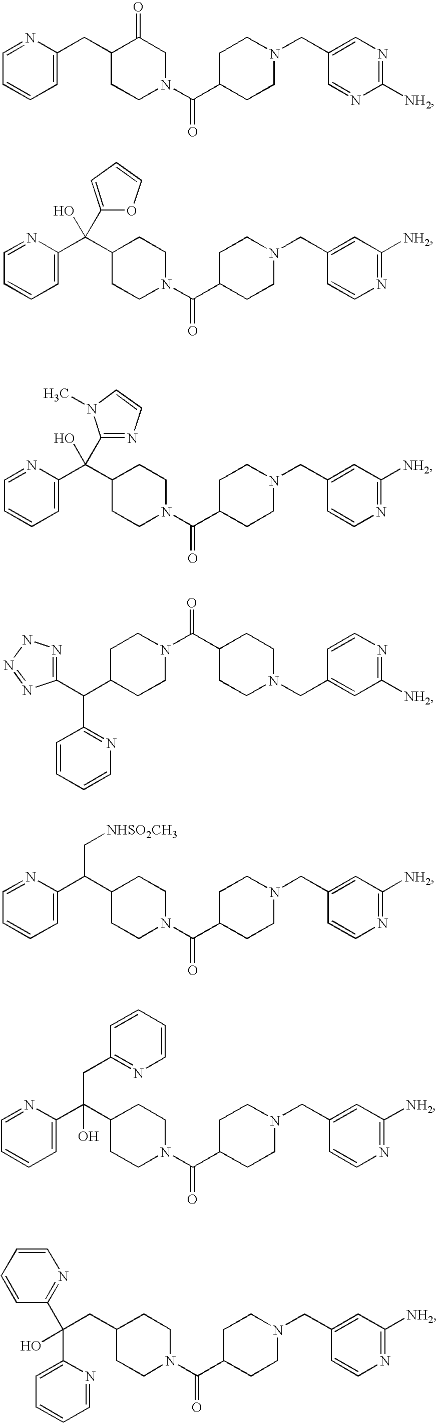

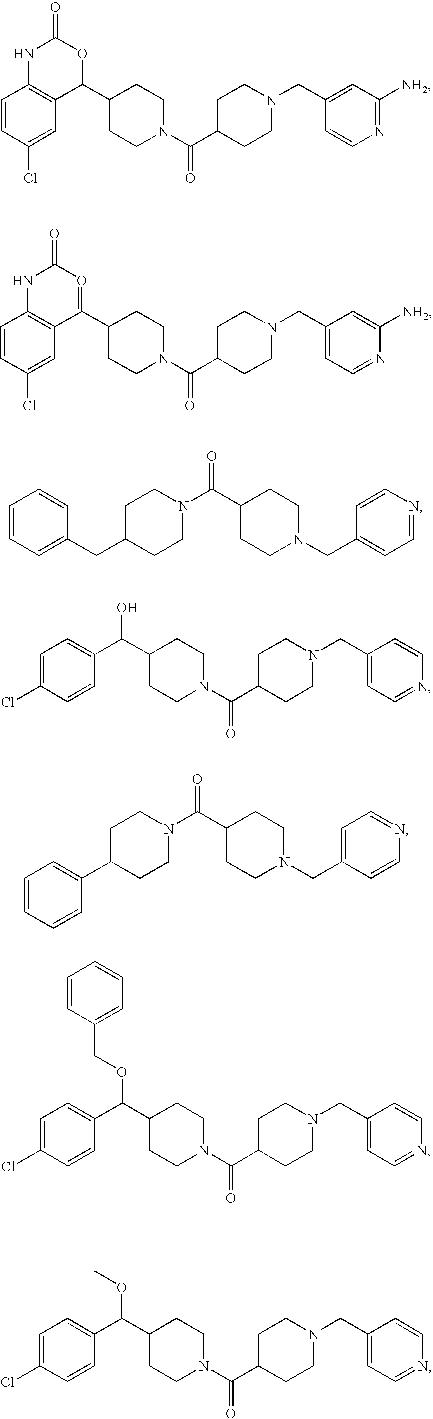

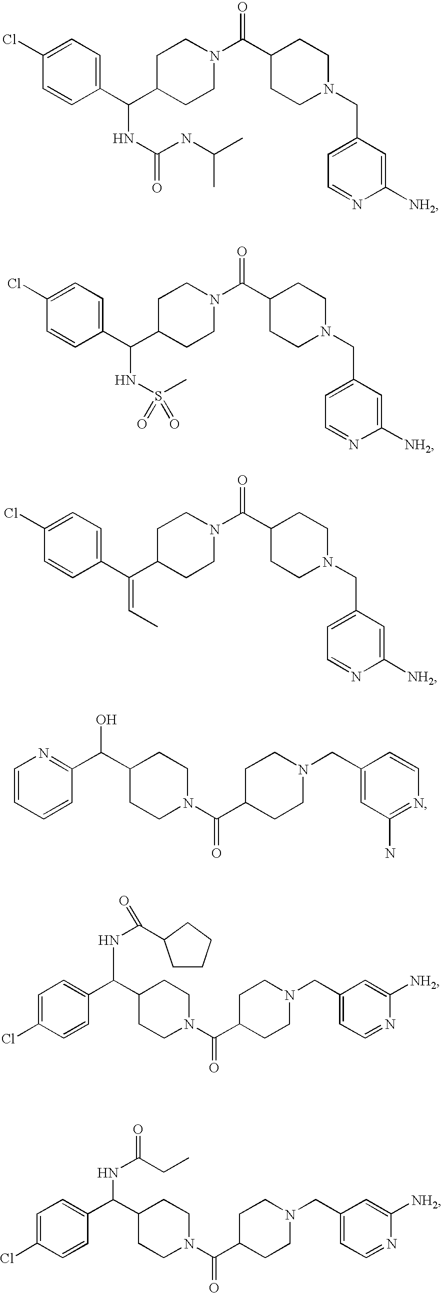

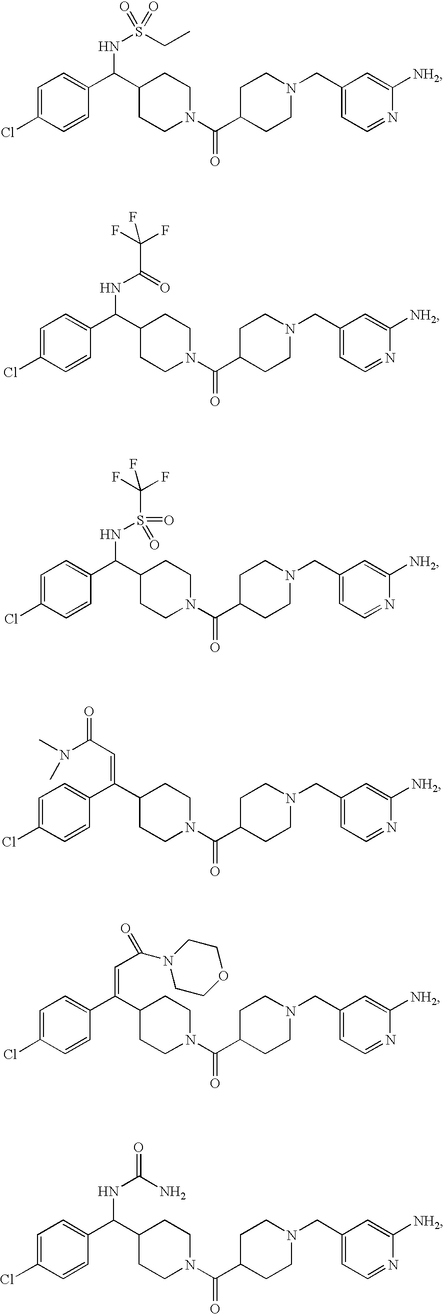

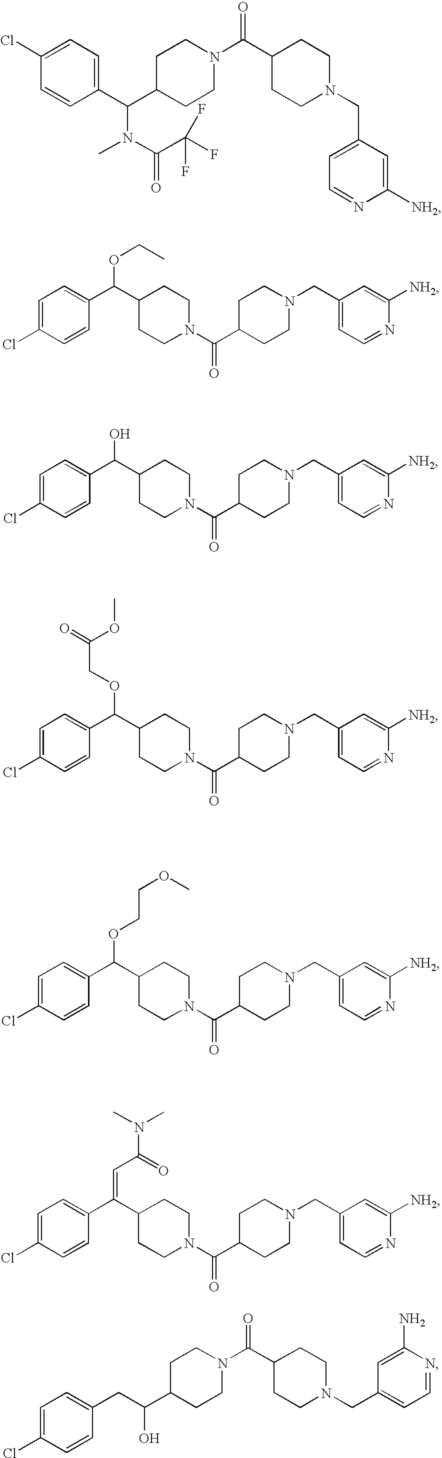

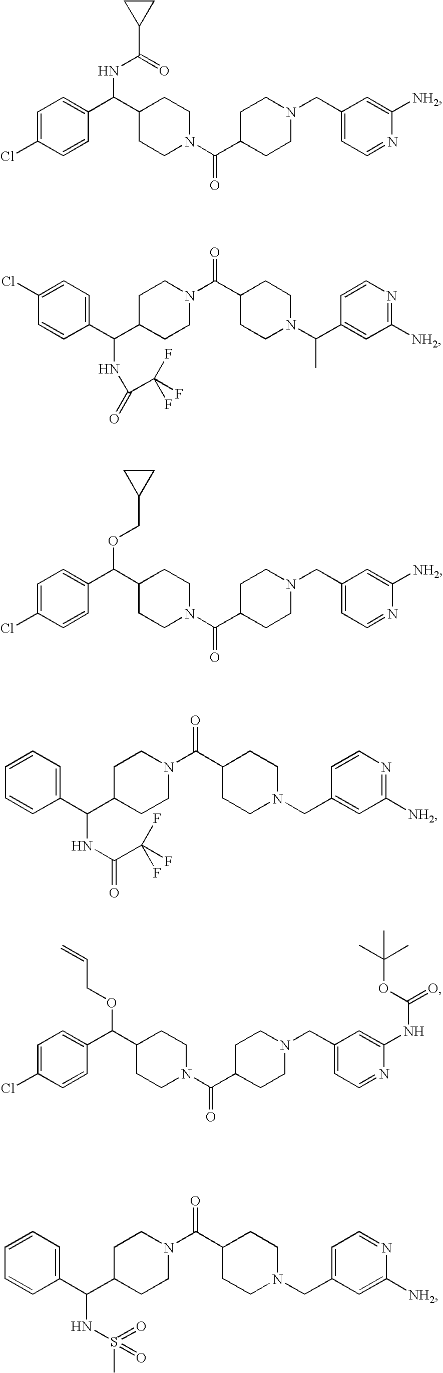

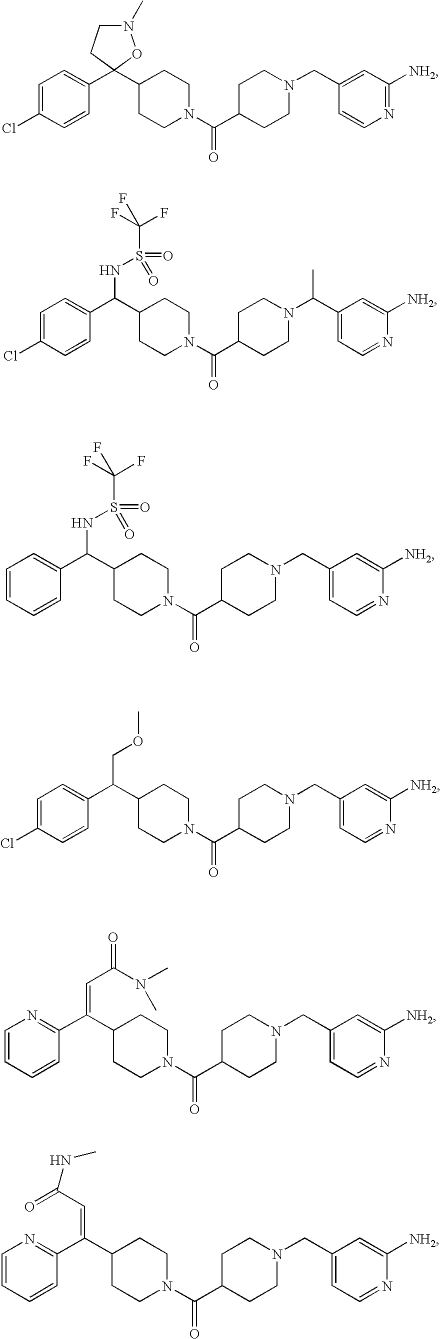

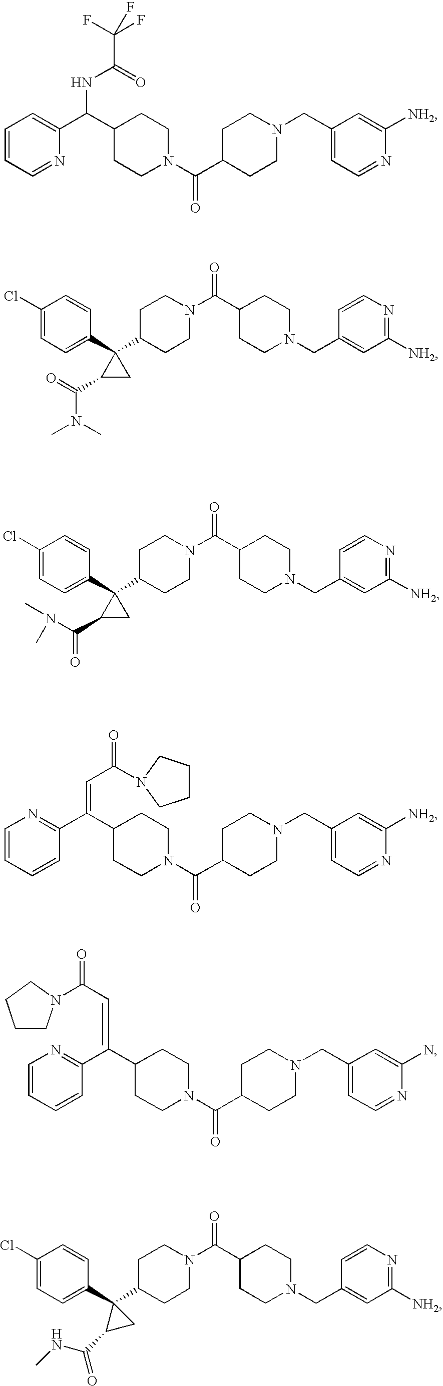

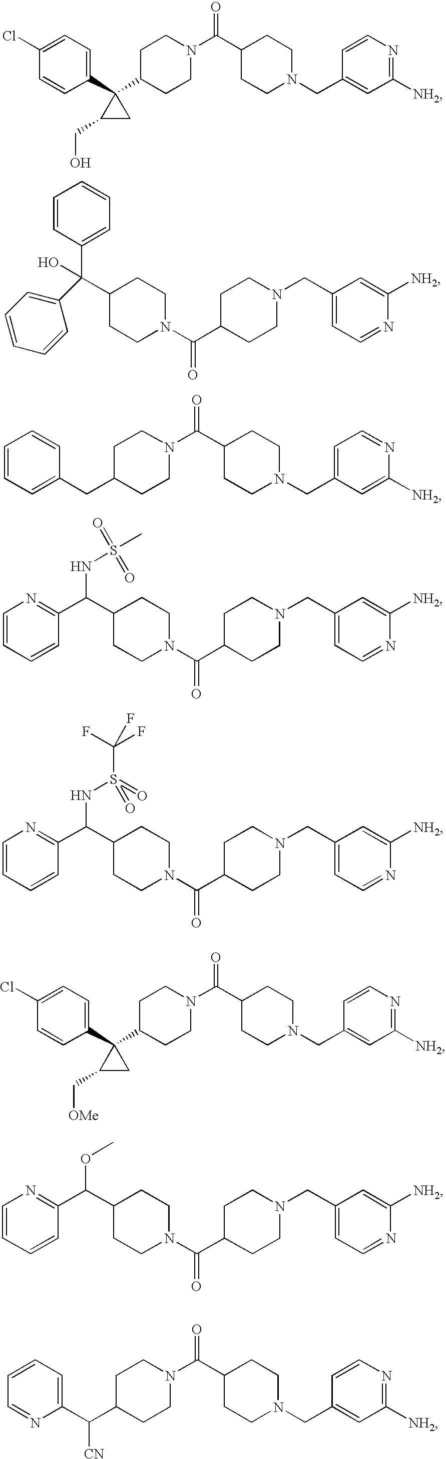

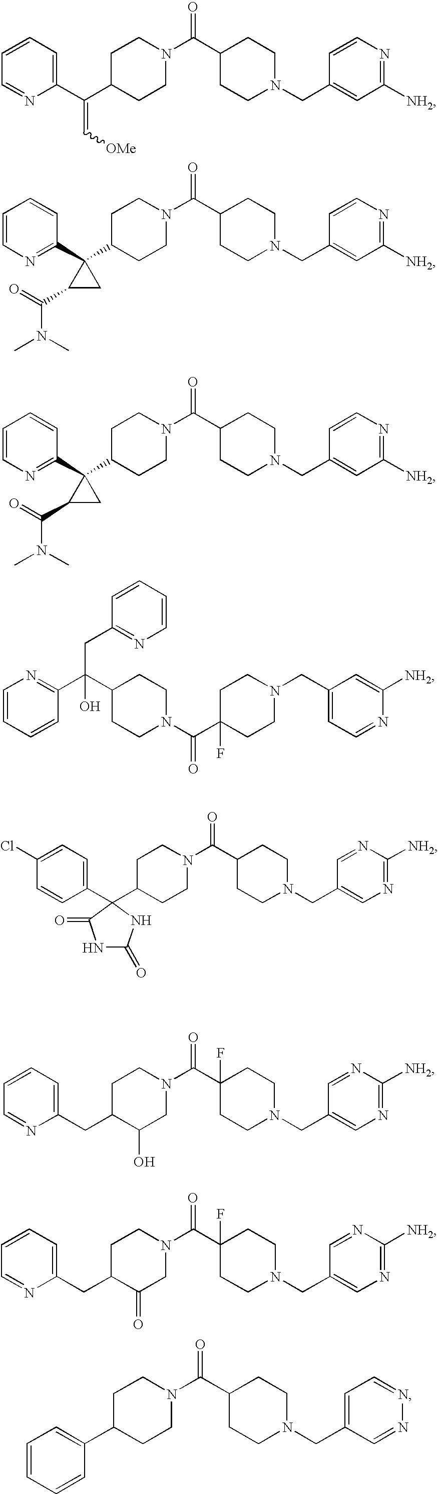

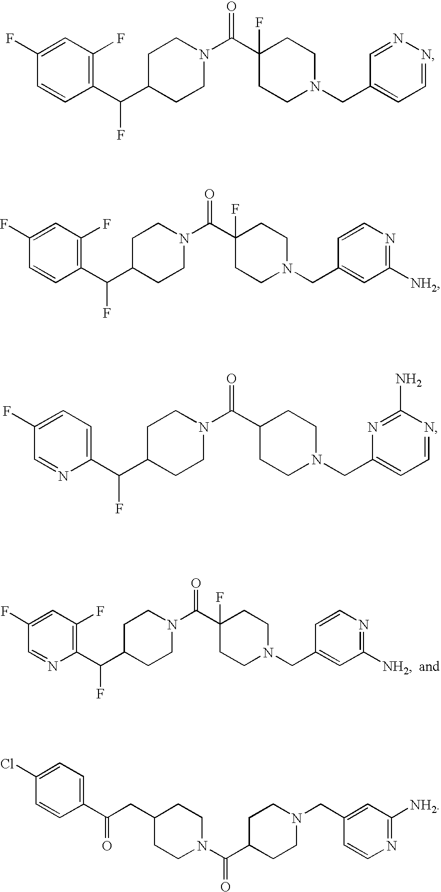

We claim:

1. A composition comprising one or more appetite suppressants and one or more metabolic rate enhancers, wherein the appetite suppressant is selected from the group consisting of a CB₁ antagonist, phentermine, sibutramine, and topiramate; and wherein the one or more metabolic rate enhancers are selected from:
(i) a compound of Formula (I): 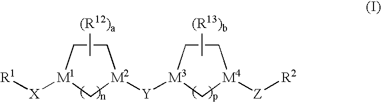
or a pharmaceutically acceptable salt, solvate, ester or prodrug thereof, wherein:
(1) R¹ is selected from:
(a) aryl;
(b) heteroaryl;
(c) heterocycloalkyl
(d) alkyl;
(e) cycloalkyl; or
(f) alkylaryl;
wherein the R¹ groups are optionally substituted with 1 to 4 substituents independently selected from:
(1) halogen;
(2) hydroxyl;
(3) lower alkoxy;
(4) —CF₃;
(5) CF₃O—;
(6) —NR⁴R⁵;
(7) phenyl;
(8) —NO₂,
(9) —CO₂R⁴;
(10) —CON(R⁴)₂ wherein each R⁴ is the same or different;
(11) —S(O)ₘN(R²⁰)₂ wherein each R²⁰ is the same or different H or alkyl group;
(12) —CN; or
(13) alkyl; or
(2) R¹ and X taken together form a group selected from: 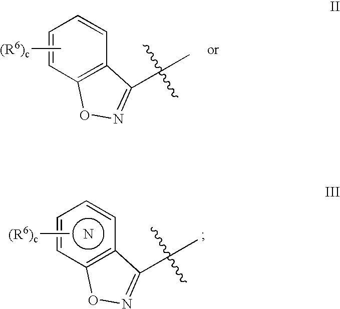
(3) X is selected from: ═C(O), ═C(NOR³), ═C(NNR⁴R⁵), 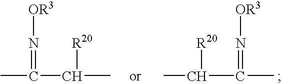
(4) M¹ is carbon;
(5) M² is selected from C or N;
(6) M³ and M⁴ are independently selected from C or N;
(7) Y is selected from: is —CH₂—, ═C(O), ═C(NOR²⁰) (wherein R²⁰ is as defined above), or ═C(S);
(8) Z is a C₁-C₆ alkyl group;
(9) R² is a five or six-membered heteroaryl ring, said six-membered heteroaryl ring comprising 1 or 2 nitrogen atoms with the remaining ring atoms being carbon, and said five-membered heteroaryl ring containing 1 or 2 heteroatoms selected from: nitrogen, oxygen, or sulfur with the remaining ring atoms being carbon; said five or six membered heteroaryl rings being optionally substituted with 1 to 3 substituents independently selected from: halogen, hydroxyl, lower alkyl, lower alkoxy, —CF₃, CF₃O—, —NR⁴R⁵, phenyl, —NO₂, —CO₂R⁴, —CON(R⁴)₂ wherein each R⁴ is the same or different, —CH₂NR⁴R⁵, —(N)C(NR⁴R⁵)₂, or —CN;
(10) R³ is selected from:
(a) hydrogen;
(b) C₁-C₆ alkyl;
(c) aryl;
(d) heteroaryl;
(e) heterocycloalkyl;
(f) arylalkyl;
(g) —(CH₂)ₑ—C(O)N(R⁴)₂ wherein each R⁴ is the same or different,
(h) —(CH₂)ₑ—C(O)OR⁴;
(i) —(CH₂)ₑ—C(O)R³⁰ wherein R³⁰ is a heterocycloalkyl group;
(j) —CF₃; or
(k) —CH₂CF₃;
wherein the aryl, heteroaryl, heterocycloalkyl, and the aryl portion of said arylalkyl are optionally substituted with 1 to 3 substituents selected from: halogen, —OH, —OCF₃, —CF₃, —CN, —N(R⁴⁵)₂, —CO₂R⁴⁵, or —C(O)N(R⁴⁵)₂, wherein each R⁴⁵ is independently selected from: H, alkyl, alkylaryl, or alkylaryl wherein the aryl moiety is substituted with 1 to 3 substituents independently selected from —CF₃, —OH, halogen, alkyl, —NO₂, or —CN;
(11) R⁴ is selected from: hydrogen, C₁-C₆ alkyl, aryl, alkylaryl, said aryl and alkylaryl groups being optionally substituted with 1 to 3 substituents selected from: halogen, —CF₃, —OCF₃, —OH, —N(R⁴⁵)₂, —CO₂R⁴⁵, —C(O)N(R⁴⁵)₂, or —CN; wherein R⁴⁵ is as defined above;
(12) R⁵ is selected from: hydrogen, C₁-C₆ alkyl, —C(O)R⁴, —C(O)₂R⁴, or —C(O)N(R⁴)₂ wherein each R⁴ is independently selected, and R⁴ is as defined above;
(13) or R⁴ and R⁵ taken together with the nitrogen atom to which they are bound forms a five or six membered heterocycloalkyl ring;
(14) R⁶ is selected from: alkyl, aryl, alkylaryl, halogen, hydroxyl, lower alkoxy, —CF₃, CF₃O—, —NR⁴R⁵, phenyl, —NO₂, —CO₂R⁴, —CON(R⁴)₂ wherein each R⁴ is the same or different, or —CN;
(15) R¹² is selected from: alkyl, hydroxyl, alkoxy, or fluoro;
(16) R¹³ is selected from: alkyl, hydroxyl, alkoxy, or fluoro;
(17) a is 0 to 2;
(18) b is 0 to 2;
(19) c is 0 to 2;
(20) e is 0 to 5;
(21) m is 1 or2;
(22) n is 1, 2 or 3; and
(23) p is 1, 2 or 3, with the proviso that when M³ and M⁴ are both nitrogen, then p is 2 or 3; or
(iI) a compound of Formula (II): 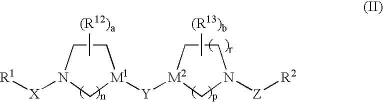
or a pharmaceutically acceptable salt, solvate, ester or prodrug thereof, wherein:
(A) R¹ is selected from:
(1) aryl;
(2) heteroaryl;
(3) heterocycloalkyl
(4) alkyl;
(5) —C(O)N(R^{4B})₂;
(6) cycloalkyl;
(7) arylalkyl;
(8) heteroarylheteroaryl; or
(9) a group selected from: 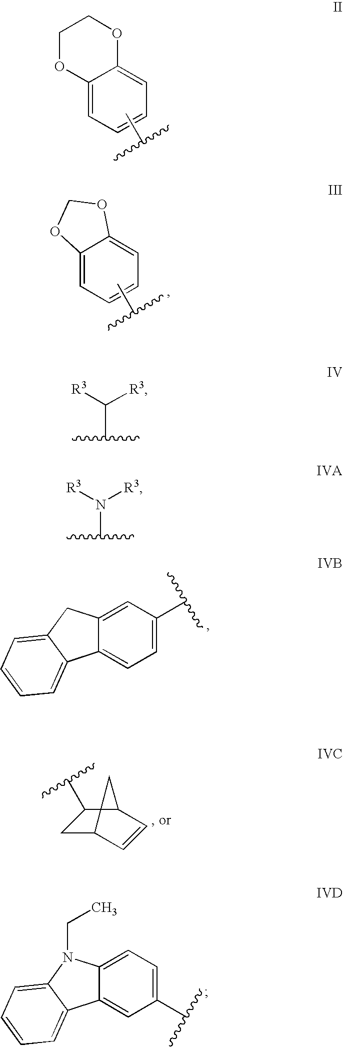
said aryl, heteroaryl, aryl portion of arylalkyl, phenyl ring of formula II, phenyl ring of formula III, phenyl rings of formula IVB, or phenyl rings of formula IVD are optionally substituted with 1 to 3 substituents independently selected from:
(1) halogen;
(2) hydroxyl;
(3) lower alkoxy;
(4) —Oaryl;
(5) —SR²²;
(6) —CF₃;
(7) —OCF₃;
(8) —OCHF₂;
(9) —NR⁴R⁵;
(10) phenyl;
(11) NO₂,
(12) —CO₂R⁴;
(13) —CON(R⁴)₂ wherein each R⁴ is the same or different;
(14) —S(O)₂R²²;
(15) —S(O)₂N(R²⁰)₂ wherein each R²⁰ is the same or different;
(16) —N(R²⁴)S(O)₂R²²;
(17) —CN;
(18) —CH₂OH;
(19) —OCH₂CH₂OR²²;
(20) alkyl;
(21) substituted phenyl wherein the phenyl has 1 to 3 substituents independently selected from alkyl, halogen, —CN, —NO₂, —OCHF₂, —Oalkyl;
(22) —Oalkylaryl wherein the aryl group is optionally substituted with 1 to 3 independently selected halogens; or
(23) phenyl;
(C) X is selected from alkyl or —S(O)₂—;
(D) Y represents
(1) a single bond; or
(2) Y is selected from —C(O)—, —C(S)—, —(CH₂)_{q}—, or —NR⁴C(O)—; with the provisos that:
(a) when M¹ is N, then Y is not —NR⁴C(O)—; and
(b) when Y is a bond, then M¹ and M² are both carbon;
(E) M¹ and M² are independently selected from C or N;
(F) Z is selected from: C₁-C₆ alkyl, —SO₂—, —C(O)— or —C(O)NR⁴—;
(G) R² is selected from:
(1) a six-membered heteroaryl ring having 1 or 2 heteroatoms independently selected from N or N—O, with the remaining ring atoms being carbon;
(2) a five-membered heteroaryl ring having 1 to 3 heteroatoms selected from nitrogen, oxygen, or sulfur with the remaining ring atoms being carbon; or
(3) an alkyl group;
(4) an aryl group wherein the substituted phenyl is substituted with 1 to 3 substituents independently selected from: halogen, —Oalkyl, —OCF₃, —CF₃, —CN, —NO₂, —NHC(O)CH₃, or —O(CH₂)_{q}N(R^{10A})₂;
(5) —N(R^{11A})₂ wherein each R^{11A} is independently selected from: H, alkyl or aryl;
(6) a group of the formula: 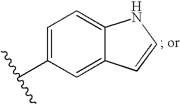
(7) a heteroarylheteroaryl group;
said five membered heteroaryl ring ((G)(2) above) or six-membered heteroaryl ring ((G)(1) above) is optionally substituted with 1 to 3 substituents selected from:
(a) halogen;
(b) hydroxyl;
(c) lower alkyl;
(d) lower alkoxy;
(e) —CF₃;
(f) —NR⁴R⁵;
(g) phenyl;
(h) —NO₂;
(i) —C(O)N(R⁴)₂ (wherein each R⁴ is the same or different);
(j) —C(O)₂R⁴; or
(k) phenyl substituted with 1 to 3 substituents independently selected from: halogen, —Oalkyl, —OCF₃, —CF₃, —CN, —NO₂ or —O(CH₂)_{q}N(R^{10A})₂;
(H) R³ is selected from:
(1) aryl;
(2) heteroaryl;
(3) heterocycloalkyl
(4) alkyl; or
(5) cycloalkyl;
wherein the aryl or heteroaryl R³ groups is optionally substituted with 1 to 3 substituents independently selected from:
(a) halogen;
(b) hydroxyl;
(c) lower alkoxy;
(d) —Oaryl;
(e) —SR²²;
(f) —CF₃;
(g) —OCF₃;
(h) —OCHF₂;
(i) —NR⁴R⁵;
(j) phenyl;
(k) —NO₂,
(l) —CO₂R⁴;
(m) —CON(R⁴)₂ wherein each R⁴ is the same or different;
(n) —S(O)₂R²²;
(o) —S(O)₂N(R²⁰)₂ wherein each R²⁰ is the same or different;
(p) —N(R²⁴)S(O)₂R²²;
(q) —CN;
(r) —CH₂OH;
(s) —OCH₂CH₂OR²²; or
(t) alkyl;
(I) R⁴ is selected from:
(1) hydrogen;
(2) C₁-C₆ alkyl;
(3) cycloalkyl;
(4) cycloalkylalkyl;
(5) heterocycloalkylalky;
(6) bridged bicyclic cycloalkyl ring;
(7) aryl having a fused heterocycloalkyl ring bound to said aryl ring;
(8) aryl;
(9) arylalkyl;
(10) alkylaryl;
(11) —(CH₂)_{d}CH(R^{12A})₂ wherein d is 1 to 3, and each R^{12A} is independently selected from phenyl or substituted phenyl, said substituted phenyl being substituted with 1 to 3 substituents independently selected from: halogen, —Oalkyl, —OCF₃, —CF₃, —CN, or —NO₂;
(12) heterocycloalkylheteroaryl; or
(13) —(C₁ to C₆)alkylene-O—R²²;
wherein the aryl R⁴ group, the aryl portion of the arylalkyl R⁴ group, or the aryl portion of the alkylaryl R⁴ group is optionally substituted with 1 to 3 substituents independently selected from:
(a) halogen;
(b) hydroxyl;
(c) lower alkyl;
(d) lower alkoxy;
(e) —CF₃;
(f) —N(R²⁰)(R²⁴),
(g) phenyl;
(h) —NO₂;
(i) —C(O)N(R²⁰)₂ (wherein each R²⁰ is the same or different),
(j) —C(O)R²²;
(i) —(CH₂)ₖ-cycloalkyl;
(j) —(CH₂)_{q}-aryl; or
(k) —(CH₂)ₘ—OR²²;
(J) each R^{4B} is independently selected from: H, heteroaryl, alkyl, alkenyl, a group of the formula 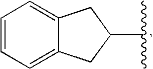
arylalkyl, or arylalkyl wherein the aryl moiety is substitued with 1-3 substituents independently selected from: halogen;
(K) R⁵ is selected from: hydrogen, C₁-C₆ alkyl, —C(O)R²⁰, —C(O)₂R²⁰, —C(O)N(R²⁰)₂ (wherein each R²⁰ is the same or different);
(L) each R^{10A} is independently selected from H or C₁ to C₆ alkyl, or each R^{10A}, taken together with the nitrogen atom to which they are bound, forms a 4 to 7 membered heterocycloalkyl ring;
(M) R¹² is
(1) selected from alkyl, hydroxyl, alkoxy, or fluoro, provided that when R¹² is hydroxy or fluoro then R¹² is not bound to a carbon adjacent to a nitrogen; or
(2) R¹² forms an alkyl bridge from one ring carbon to another ring carbon;
(N) R¹³ is
(1) selected from alkyl, hydroxyl, alkoxy, or fluoro, provided that when R¹³ is hydroxy or fluoro then R¹³ is not bound to a carbon adjacent to a nitrogen; or
(2) R¹³ forms an alkyl bridge from one ring carbon to another ring carbon;
(O) R²⁰ is selected from hydrogen, alkyl, or aryl, wherein the aryl group is optionally substituted with from 1 to 3 groups independently selected from: halogen, —CF₃, —OCF₃, hydroxyl, or methoxy; or when two R²⁰ groups are present, said two R²⁰ groups taken together with the nitrogen to which they are bound form a five or six membered heterocyclic ring;
(P) R²² is selected from: heterocycloalkyl, alkyl or aryl, wherein the aryl group is optionally substituted with 1 to 3 groups independently selected from halogen, —CF₃, —OCF₃, hydroxyl, or methoxy;
(Q) R²⁴ is selected from: hydrogen, alkyl, —SO₂R²², or aryl, wherein the aryl group is optionally substituted with 1 to 3 groups independently selected from halogen, —CF₃, —OCF₃, hydroxyl, or methoxy;
(R) a is 0 to 2;
(S) b is 0 to 2;
(T) k is 1 to 5;
(U) m is 2 to 5;
(V) n is 1, 2 or 3 with the proviso that when M¹ is N, then n is not 1;
(W) p is 1, 2 or 3 with the proviso that when M² is N, then p is not 1;
(X) q is 1 to 5; and
(Y) r is 1, 2, or 3 with the proviso that when r is 2 or 3, then M² is C and p is 1; or
(iii) a compound of Formula (III): 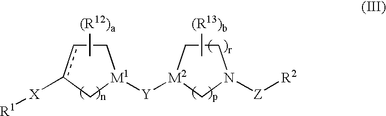
or a pharmaceutically acceptable salt, solvate, ester or prodrug thereof, wherein:
the dotted line represents an optional double bond;
a is 0 to 2;
b is 0 to 2;
n is 1, 2 or 3;
p is 1, 2 or 3;
r is 0, 1, 2, or 3;
with the provisos that when M² is N, p is not 1; and that when r is 0, M² is C(R³); and that the sum of p and r is 1 to 4;
M¹ is C(R³) or N;
M² is C(R³) or N;
X is a bond or C₁-C₆ alkylene;
Y is —C(O)—, —C(S)—, —(CH₂)_{q}, —NR⁴C(O)—, —C(O)NR⁴—, —C(O)CH₂—, —SO₂—, —N(R⁴)—, —NH—C(═N—CN)— or —C(═N—CN)—NH—; with the provisos that when M¹ is N, Y is not —NR⁴C(O)— or —NH—C(═N—CN)—; when M² is N, Y is not —C(O)NR⁴— or —C(═N—CN)—NH—; and when Y is —N(R⁴)—, M¹ is CH and M² is C(R³);
q is 1 to 5, provided that when both M¹ and M² are N, q is 2 to 5;
Z is a bond, C₁-C₆ alkylene, C₁-C₆ alkenylene, —C(O)—, —CH(CN)—, —SO₂— or —CH₂C(O)N R⁴—;
R¹ is 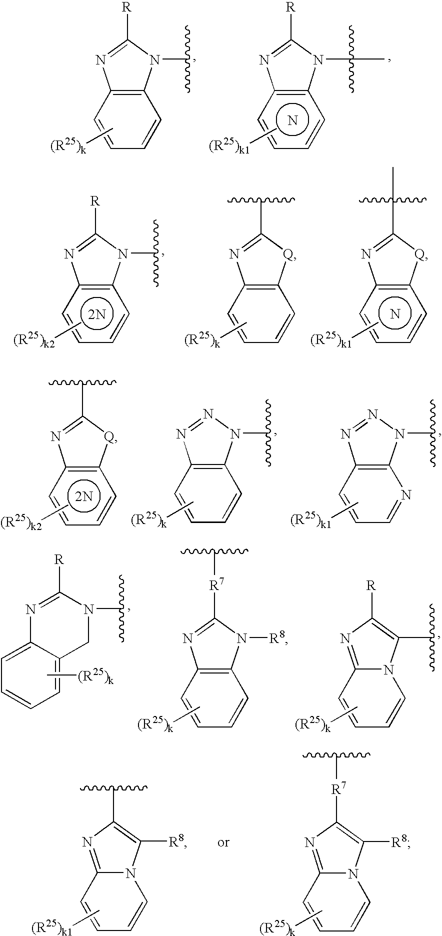
Q is —N(R⁸)—, —S— or —O—;
k is 0, 1, 2, 3or 4;
k1 is 0, 1, 2 or 3;
k2 is 0, 1 or 2;
R is H, C₁-C₆ alkyl, halo(C₁-C₆)alkyl-, C₁-C₆ alkoxy, (C₁-C₆)alkoxy-(C₁-C₆)alkyl-, (C₁-C₆)-alkoxy-(C₁-C₆)alkoxy, (C₁-C₆)alkoxy-(C₁-C₆)alkyl-SO₀₋₂, R³²-aryl(C₁-C₆)alkoxy-, R³²-aryl(C₁-C₆)alkyl-, R³²-aryl, R³²-aryloxy, R³²-heteroaryl, (C₃-C₆)cycloalkyl, (C₃-C₆)cycloalkyl-(C₁-C₆)alkyl, (C₃-C₆)cycloalkyl-(C₁-C₆)alkoxy, (C₃-C₆)cycloalkyl-oxy-, R³⁷-heterocycloalkyl, R³⁷-heterocycloalkyl-oxy-, R³⁷-heterocycloalkyl-(C₁-C₆)alkoxy, N(R³⁰)(R³¹)-(C₁-C₆)alkyl-, —N(R³⁰)(R³¹), —NH—(C₁-C₆)alkyl-O-(C₁-C₆)alkyl, —NHC(O)NH(R²⁹); R²⁹—S(O)₀₋₂—, halo(C₁-C₆)alkyl-S(O)₀₋₂—, N(R³⁰)(R³¹)=(C₁-C₆)alkyl-S(O)₀₋₂— or benzoyl;
R⁸ is H, C₁-C₆ alkyl, halo(C₁-C₆)alkyl-, (C₁-C₆)alkoxy-(C₁-C₆)alkyl-, R³²-aryl(C₁-C₆)alkyl-, R³²-aryl, R³²-heteroaryl, (C₃-C₆)cycloalkyl, (C₃-C₆)cycloalkyl-(C₁-C₆alkyl, R³⁷-heterocycloalkyl, N(R³⁰)(R³¹)—(C₁-C₆)alkyl-, R²⁹—S(O)₂—, halo(C₁-C₆)alkyl-S(O)₂—, R²⁹—S(O)₀₋₁—(C₂-C₆)alkyl-, halo(C₁-C₆)alkyl-S(O)₀₋₁—(C₂-C₆)alkyl-;
R² is a six-membered heteroaryl ring having 1 or 2 heteroatoms independently selected from N or N-O, with the remaining ring atoms being carbon; a five-membered heteroaryl ring having 1, 2, 3 or 4 heteroatoms independently selected from N, O or S, with the remaining ring atoms being carbon; R³²-quinolyl; R³²-aryl; heterocycloalkyl; (C₃-C₆)cycloalkyl; C₁-C₆ alkyl; hydrogen; thianaphthenyl; 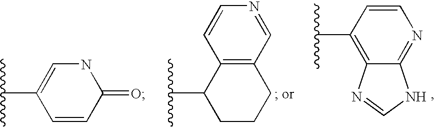
wherein the six-membered heteroaryl ring or said five-membered heteroaryl ring is optionally substituted by R⁶;
R³ is H, halogen, C₁-C₆ alkyl, —OH, (C₁-C₆)alkoxy or —NHSO₂—(C₁-C₆)alkyl;
R⁴ is independently selected from the group consisting of hydrogen, C₁-C₆ alkyl, C₃-C₆ cycloalkyl, (C₃-C₆)cycloalkyl(C₁-C₆)alkyl, R³³-aryl, R³³-aryl(C₁-C₆)alkyl, and R³²-heteroaryl;
R⁵ is hydrogen, C₁-C₆ alkyl, —C(O)R²⁰, —C(O)₂R²⁰, —C(O)N(R²⁰)₂, (C₁-C₆alkyl-SO₂—, or (C₁-C₆)alkyl-SO₂—NH—;
or R⁴ and R⁵, together with the nitrogen to which they are attached, form an azetidinyl, pyrrolidinyl, piperidinyl, piperazinyl or morpholinyl ring;
R⁶ is 1 to 3 substituents independently selected from the group consisting of —OH, halogen, C₁-C₆ alkyl-, C₁-C₆ alkoxy, C₁-C₆ alkylthio, —CF₃, —NR⁴R⁵, —CH₂—NR⁴R⁵, —NHSO₂R²², —N(SO₂R²²)₂, phenyl, R³³-phenyl, NO₂, —CO₂R⁴, —CON(R⁴)₂, 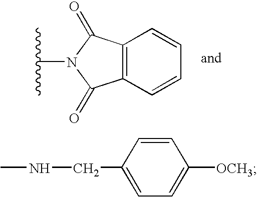
R⁷ is —N(R²⁹)—, —O— or —S(O)₀₋₂—;
R¹² is independently selected from the group consisting of C₁-C₆ alkyl, hydroxyl, C₁-C₆ alkoxy, or fluoro, provided that when R¹² is hydroxy or fluoro, then R¹² is not bound to a carbon adjacent to a nitrogen; or two R¹² substituents form a C₁ to C₂ alkyl bridge from one ring carbon to another non-adjacent ring carbon; or R¹² is ═O;
R¹³ is independently selected from the group consisting of C₁-C₆ alkyl, hydroxyl, C₁-C₆ alkoxy, or fluoro, provided that when R¹³ is hydroxy or fluoro then R¹³ is not bound to a carbon adjacent to a nitrogen; or two R¹³ substituents form a C₁ to C₂ alkyl bridge from one ring carbon to another non-adjacent ring carbon; or R¹³ is ═O;
R²⁰ is independently selected from the group consisting of hydrogen, C₁-C₆ alkyl, or aryl, wherein the aryl group is optionally substituted with from 1 to 3 groups independently selected from halogen, —CF₃, —OCF₃, hydroxyl, or methoxy; or when two R²⁰ groups are present, said two R²⁰ groups taken together with the nitrogen to which they are bound can form a five or six membered heterocyclic ring;
R²² is C₁-C₆ alkyl, R³⁴-aryl or heterocycloalkyl;
R²⁴ is H, C₁-C₆ alkyl, —SO₂R²² or R³⁴-aryl;
R²⁵ is independently selected from the group consisting of C₁-C₆ alkyl, halogen, —CN, —NO₂, —CF₃, —OH, C₁-C₆ alkoxy, (C₁-C₆)alkyl-C(O)—, aryl-C(O)—, —C(O)OR²⁹, —N(R⁴)(R⁵), N(R⁴)(R⁵)—C(O)—, N(R⁴)(R⁵)—S(O)₁₋₂—, R²²—S(O)₀₋₂—, halo-(C₁-C₆)alkyl- or halo-(C₁-C₆)alkoxy-(C₁-C₆)alkyl-;
R²⁹ is H, C₁-C₆ alkyl, C₃-C₆ cycloalkyl, R³⁵-aryl or R³⁵-aryl(C₁-C₆)alkyl-;
R³⁰ is H, C₁-C₆ alkyl-, R³⁵-aryl or R³⁵-aryl(C₁-C₆)alkyl-;
R³¹ is H, C₁-C₆ alkyl-, R³⁵-aryl, R³⁵-aryl(C₁-C₆)alkyl-, R³⁵-heteroaryl, (C₁-C₆)alkyl-C(O)—, R³⁵-aryl-C(O)—, N(R⁴)(R⁵)—C(O)—, (C₁-C₆)alkyl-S(O)₂— or R³⁵-aryl-S(O)₂—;
or R³⁰ and R³¹ together are —(CH₂)₄₋₅—, —(CH₂)₂—O—(CH₂)₂— or —(CH₂)₂—N(R³⁸)—(CH₂)₂— and form a ring with the nitrogen to which they are attached;
R³² is 1 to 3 substituents independently selected from the group consisting of H, —OH, halogen, C₁-C₆ alkyl, C₁-C₆ alkoxy, R³⁵-aryl-O—, —SR²², —CF₃, —OCF₃, —OCHF₂, —NR³⁹R⁴⁰, phenyl, R³³-phenyl, NO₂, —CO₂R³⁹, —CON(R³⁹)₂, —S(O)₂R²², —S(O)₂N(R²⁰)₂, —N(R²⁴)S(O)₂R²², —CN, hydroxy-(C₁-C₆)alkyl-, —OCH₂CH₂OR²², and R³⁵-aryl(C₁-C₆)alkyl-O—, or two R³² groups on adjacent carbon atoms together form a —OCH₂O— or —O(CH₂)₂O— group;
R³³ is 1 to 3 substituents independently selected from the group consisting of C₁-C₆ alkyl, halogen, —CN, —NO₂, —CF₃, —OCF₃, —OCHF₂ and —O—(C₁-C₆)alkyl;
R³⁴ is 1 to 3 substituents independently selected from the group consisting of H, halogen, —CF₃, —OCF₃, —OH and —OCH₃;
R³⁵ is 1 to 3 substituents independently selected from hydrogen, halo, C₁-C₆ alkyl, hydroxy, C₁-C₆ alkoxy, phenoxy, —CF₃, —N(R³⁶)₂, —COOR²⁰ and —NO₂;
R³⁶ is independently selected form the group consisting of H and C₁-C₆ alkyl;
R³⁷ is 1 to 3 substituents independently selected from hydrogen, halo, C₁-C₆ alkyl, hydroxy, C₁-C₆ alkoxy, phenoxy, —CF₃, —N(R³⁶)₂, —COOR²⁰, —C(O)N(R²⁹)₂ and —NO₂, or R³⁷ is one or two ═O groups;
R³⁸ is H, C₁-C₆ alkyl, R³⁵-aryl, R³⁵-aryl(C₁-C₆)alkyl-, (C₁-C₆)alkyl-SO₂ or halo(C₁-C₆)alkyl-SO₂—;
R³⁹ is independently selected from the group consisting of hydrogen, C₁-C₆ alkyl, C₃-C₆ cycloalkyl, (C₃-C₆)cycloalkyl(C₁-C₆)alkyl, R³³-aryl, R³³-aryl(C₁-C₆)alkyl, and R³²-heteroaryl; and
R⁴⁰ is hydrogen, C₁-C₆ alkyl, —C(O)R²⁰, —C(O)₂R²⁰, —C(O)N(R²⁰)₂, (C₁-₆)alkyl-SO₂—, or (C₁-C₆)alkyl-SO₂—NH—;
or R³⁹ and R⁴⁰, together with the nitrogen to which they are attached, form an azetidinyl, pyrrolidinyl, piperidinyl, piperazinyl or morpholinyl ring; or
(iv) a compound of Formula (IV): 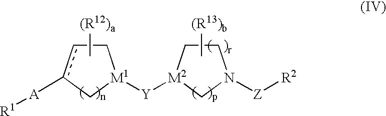
or a pharmaceutically acceptable salt, solvate, ester or prodrug thereof, wherein:
the dotted line represents an optional double bond;
a is 0 to 3;
b is 0 to 3;
n is 1, 2 or 3;
p is 1, 2 or 3 with the proviso that when M² is N, then p is not 1;
r is 1, 2, or 3 with the proviso that when r is 2 or 3, then M² is C(R³) and p is 2 or 3;
A is a bond or C₁-C₆ alkylene;
M¹ is C(R³) or N;
M² is C(R³) or N;
Y is —C(═O)—, —C(═S)—, —(CH₂)_{q}—, —NR⁴C(═O)—, —C(═O)NR⁴—, —C(═O)CH₂—, —CH₂(C═O)—, —SO₁₋₂—, —NH—C(═N—CN)— or —C(═N—CN)—NH—; with the provisos that when M¹ is N, Y is not —NR⁴C(═O)— or —NH—C(═N—CN)—; and when M² is N, Y is not —C(═O)NR⁴— or —C(═N—CN)—NH—;
q is 1 to 5, provided that when M¹ and M² are both N, q is not 1;
Z is a bond, C₁-C₆ alkylene, C₁-C₆ alkenylene, —C(═O)—, —CH(CN)—, or —CH₂C(═O)NR⁴—;
R¹ is 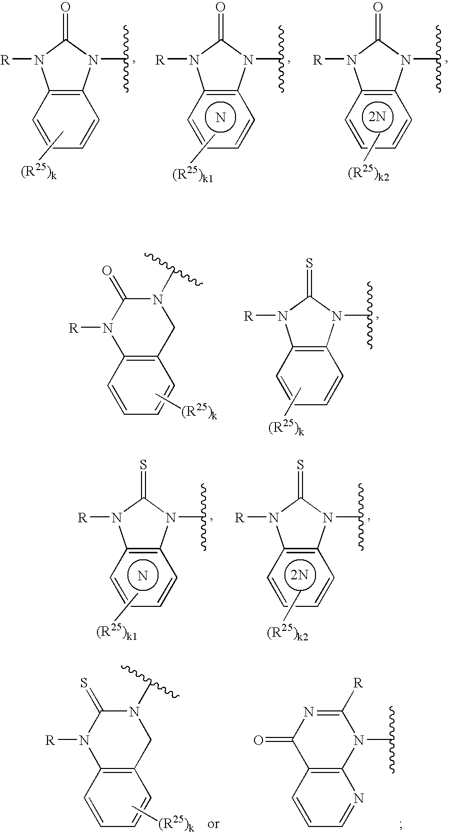
k is 0, 1, 2, 3 or 4;
k1 is 0, 1, 2 or 3;
k2 is 0, 1 or 2;
R is H, C₁-C₆ alkyl, hydroxy-(C₂-C₆)alkyl-, halo-(C₁-C₆)alkyl-, halo-(C₁-C₆)-alkoxy-(C₁-C₆)alkyl-, R²⁹—O—C(O)—(C₁-C₆)alkyl-, (C₁-C₆)alkoxy-(C₁-C₆)alkyl-, N(R³⁰)(R³¹)—(C₁-C₆)alkyl-, (C₁-C₆)alkoxy-(C₁-C₆)alkoxy-(C₁-C₆)alkyl-, R³²-aryl, R³²-aryl(C₁-C₆)alkyl-, R³²-aryloxy(C₁-C₆)alkyl-, R³²-heteroaryl, R³²-heteroaryl(C₁-C₆)alkyl-, (C₃-C₆)cycloalkyl, (C₃-C₆)cycloalkyl(C₁-C₆)alkyl-, N(R³⁰)(R³¹)—C(O)—(C₁-C₆)alkyl-, or heterocycloalkyl(C₁-C₆)alkyl-;
R² is a six-membered heteroaryl ring having 1 or 2 heteroatoms independently selected from N or N—O, with the remaining ring atoms being carbon; a five-membered heteroaryl ring having 1, 2, 3 or 4 heteroatoms independently selected from N, O or S, with the remaining ring atoms being carbon; R³²-quinolyl; R³²-aryl; heterocycloalkyl; (C₃-C₆)cycloalkyl; (C₁-C₆)alkyl; hydrogen; 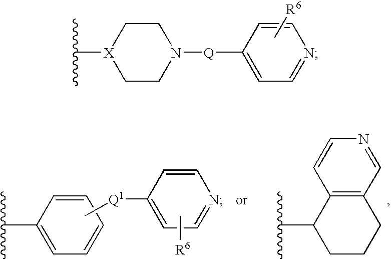
wherein the six-membered heteroaryl ring or said five-membered heteroaryl ring is optionally substituted by R⁶;
X is CH or N;
Q is a bond or C₁-C₆ alkylene;
Q¹ is a bond, C₁-C₆ alkylene or —N(R⁴)—;
R³ is H, halogen, C₁-C₆ alkyl, —OH or (C₁-C₆)alkoxy;
R⁴ is independently selected from the group consisting of hydrogen, C₁-C₆ alkyl, C₃-C₆ cycloalkyl, (C₃-C₆)cycloalkyl(C₁-C₆)alkyl, R³³-aryl, R³³-aryl(C₁-C₆)alkyl, and R³²-heteroaryl;
R⁵ is hydrogen, C₁-C₆ alkyl, —C(O)R²⁰, —C(O)₂R²⁰, —C(O)N(R²⁰)₂ or (C₁-C₆)alkyl-SO₂—;
or R⁴ and R⁵, together with the nitrogen to which they are attached, form an azetidinyl, pyrrolidinyl, piperidinyl, piperazinyl or morpholinyl ring;
R⁶ is 1 to 3 substituents independently selected from the group consisting of —OH, halogen, C₁-C₆ alkyl-, C₁-C₆ alkoxy, C₁-C₆ alkylthio, —CF₃, —NR⁴R⁵, NO₂, —CO₂R⁴, —CON(R⁴)₂, —CH₂—NR⁴R⁵, —CN, 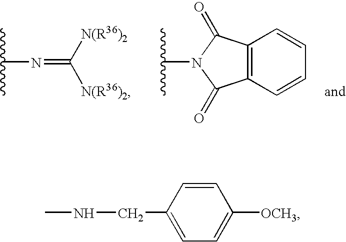
or 2 R⁶ substituents together on the same carbon are ═O;
R¹² is independently selected from the group consisting of C₁-C₆ alkyl, hydroxy, C₁-C₆ alkoxy, or fluoro, provided that when R¹² is hydroxy or fluoro, then R¹² is not bound to a carbon adjacent to a nitrogen; or two R¹² substituents together form a C₁ to C₂ alkyl bridge from one ring carbon to another non-adjacent ring carbon; or R¹² is ═O;
R¹³ is independently selected from the group consisting of C₁-C₆ alkyl, hydroxy, C₁-C₆ alkoxy, or fluoro, provided that when R¹³ is hydroxy or fluoro then R¹³ is not bound to a carbon adjacent to a nitrogen; or two R¹³ substituents together form a C₁ to C₂ alkyl bridge from one ring carbon to another non-adjacent ring carbon; or R¹³ is ═O;
R²⁰ is independently selected from the group consisting of hydrogen, C₁-C₆ alkyl, or aryl, wherein the aryl group is optionally substituted with from 1 to 3 groups independently selected from halogen, —CF₃, —OCF₃, hydroxyl, or methoxy; or when two R²⁰ groups are present, said two R²⁰ groups taken together with the nitrogen to which they are bound can form a five or six membered heterocyclic ring;
R²² is C₁-C₆ alkyl, R³⁴-aryl or heterocycloalkyl;
R²⁴ is H, C₁-C₆ alkyl, —SO₂R²² or R³⁴-aryl;
R²⁵ is independently selected from the group consisting of C₁-C₆ alkyl, —CN, —NO₂, halogen, —CF₃, —OH, C₁-C₆ alkoxy, (C₁-C₆)alkyl-C(O)—, aryl-C(O)—, N(R⁴)(R⁵)—C(O)—, N(R⁴)(R⁵)—S(O)₁₋₂—, halo-(C₁-C₆)alkyl- or halo-(C₁-C₆)alkoxy-(C₁-C₆)alkyl-;
R²⁹ is H, C₁-C₆ alkyl, R³⁵-aryl or R³⁵-aryl(C₁-C₆)alkyl-;
R³⁰ is H, C₁-C₆ alkyl-, R³⁵-aryl or R³⁵-aryl(C₁-C₆)alkyl-;
R³¹ is H, C₁-C₆ alkyl-, R³⁵-aryl, R³⁵-aryl(C₁-C₆)alkyl-, (C₁-C₆)alkyl-C(O)—, R³⁵-aryl-C(O)—, N(R⁴)(R⁵)—C(O)—, (C₁-C₆)alkyl-S(O)₂— or R³⁵-aryl-S(O)₂—;
or R³⁰ and R³¹together are —(CH₂)₄₋₅—, —(CH₂)₂—O—(CH₂)₂— or —(CH₂)₂—N(R²⁹)—(CH₂)₂— and form a ring with the nitrogen to which they are attached;
R³² is 1 to 3 substituents independently selected from the group consisting of H, —OH, halogen, C₁-C₆ alkyl, C₁-C₆ alkoxy, —SR²², —CF₃, —OCF₃, —OCHF₂, —NR³⁷R³⁸, —NO₂, —CO₂R³⁷, —CON(R³⁷)₂, —S(O)₂R²², —S(O)₂N(R²⁰)₂, —N(R²⁴)S(O)₂R²², —CN, hydroxy-(C₁-C₆)alkyl- and —OCH₂CH₂OR²²;
R³³ is 1 to 3 substituents independently selected from the group consisting of C₁-C₆ alkyl, halogen, —CN, —NO₂, —OCHF₂ and —O—(C₁-C₆)alkyl;
R³⁴ is 1 to 3 substituents independently selected from the group consisting of H, halogen, —CF₃, —OCF₃, —OH and —OCH₃;
R³⁵ is 1 to 3 substituents independently selected from hydrogen, halo, C₁-C₆ alkyl, hydroxy, C₁-C₆ alkoxy, phenoxy, —CF₃, —N(R³⁶)₂, —COOR²⁰ and —NO₂;
R³⁶ is independently selected form the group consisting of H and C₁-C₆ alkyl;
R³⁷ is independently selected from the group consisting of hydrogen, C₁-C₆ alkyl, C₃-C₆ cycloalkyl, (C₃-C₆)cycloalkyl(C₁-C₆)alkyl, R³³-aryl, R³³-aryl(C₁-C₆alkyl, and R³²-heteroaryl; and
R³⁸ is hydrogen, C₁-C₆ alkyl, —C(O)R²⁰, —C(O)₂R²⁰, —C(O)N(R²⁰)₂ or (C₁-C₆)alkyl-SO₂—;
or R³⁷ and R³⁸, together with the nitrogen to which they are attached, form an azetidinyl, pyrrolidinyl, piperidinyl, piperazinyl or morpholinyl ring; or
(v) a compound of Formula (V): 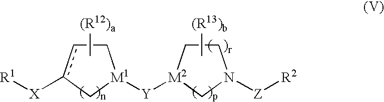
or a pharmaceutically acceptable salt, solvate, ester or prodrug thereof, wherein:
a is 0 to 3;
b is 0 to 3;
n is 1, 2 or 3;
p is 1, 2 or 3;
r is 0, 1, 2, or 3;
X is a bond or C₁-C₆ alkylene;
M¹ is CH or N;
M² is C(R³) or N;
with the provisos that when M² is N, p is not 1; and that when r is 0, M² is C(R³); and that the sum of p and r is 1 to 4;
Y is —C(═O)—, —C(═S)—, —(CH₂)_{q}—, —NR⁴C(═O)—, —C(═O)NR⁴—, —C(═O)CH₂—, —SO₁₋₂—, —C(═N—CN)—NH— or —NH—C(═N—CN)—; with the provisos that when M¹ is N, Y is not —NR⁴C(═O)— or —NH—C(═N—CN)—; and when M² is N, Y is not —C(═O)NR⁴— or —C(═N—CN)—NH—;
q is 1 to 5, provided that when M¹ and M² are both N, q is not 1;
Z is a bond, C₁-C₆ alkylene, C₂-C₆ alkenylene, —C(═O)—, —CH(CN)— or —CH₂C(═O)NR⁴—;
R¹ is 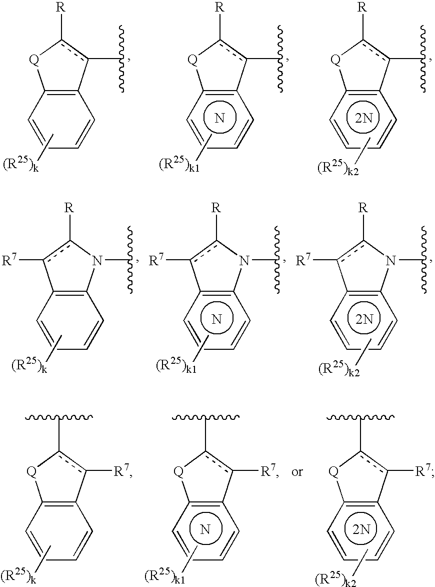
Q is —N(R⁸)—, —S— or —O—;
k is 0, 1, 2, 3 or 4;
k1 is 0, 1, 2 or 3;
k2 is 0, 1 or 2;
the dotted line represents an optional double bond;
R and R⁷ are independently selected from the group consisting of H, C₁-C₆ alkyl, halo(C₁-C₆)alkyl-, C₁-C₆ alkoxy, (C₁-C₆)alkoxy-(C₁-C₆)alkyl-, (C₁-C₆)-alkoxy-(C₁-C₆)alkoxy, (C₁-C₆)alkoxy-(C₁-C₆)alkyl-SO₀₋₂, R³²-aryl(C₁-C₆)alkoxy-, R³²-aryl-(C₁-C₆)alkyl-, R³²-aryl, R³²-aryloxy, R³²-heteroaryl, (C₃-C₆)cycloalkyl, (C₃-C₆)cycloalkyl-(C₁-C₆)alkyl, (C₃-C₆)cycloalkyl-(C₁-C₆)alkoxy, (C₃-C₆)cycloalkyl-oxy-, R³⁷-heterocyclo-alkyl, N(R³⁰)(R³¹)—(C₁-C₆)alkyl-, —N(R³⁰)(R³¹), —NH—(C₁-C₆)alkyl-O—(C₁-C₆)alkyl, —NHC(O)NH(R²⁹); R²²—S(O)₀₋₂—, halo(C₁-C₆)alkyl-S(O)₀₋₂—, N(R³⁰)(R³¹)—(C₁-C₆)alkyl-S(O)₀₋₂—, benzoyl, (C₁-C₆)alkoxy-carbonyl, R³⁷-heterocycloalkyl-N(R²⁹)—C(O)—, (C₁-C₆)alkyl-N(R²⁹)—C(O)—, (C₁-C₆)alkyl-N(C₁-C₆ alkoxy)-C(O)—, —C(═NOR³⁶)R³⁶ and —NHC(O)R²⁹; and when the optional double bond is not present, R⁷ can be OH;
R⁸ is H, C₁-C₆ alkyl, halo(C₁-C₆)alkyl-, (C₁-C₆)alkoxy-(C₂-C₆)alkyl-, R³²-aryl(C₁-C₆)alkyl-, R³²-aryl, R³²-heteroaryl, R³²-heteroaryl(C₁-C₆)alkyl-, (C₃-C₆)cycloalkyl, (C₃-C₆)cycloalkyl-(C₁-C₆)alkyl, R³⁷-heterocycloalkyl, R³⁷-heterocycloalkyl(C₁-C₆)alkyl, N(R³ )(R³¹)—(C₂-C₆)alkyl-, R²²-S(O)₂-, halo(C₁-C₆)alkyl-S(O)₂—, R²²-S(O)₀₋₁—(C₂-C₆)alkyl-, halo(C₁-C₆)alkyl-S(O)₀₋₁—(C₂-C₆)alkyl-, (C₁-C₆)alkyl-N(R²⁹)—SO₂—, or R³²-heteroaryl-SO₂;
R² is a six-membered heteroaryl ring having 1 or 2 heteroatoms independently selected from N or N—O, with the remaining ring atoms being carbon; a five-membered heteroaryl ring having 1, 2, 3 or 4 heteroatoms independently selected from N, O or S, with the remaining ring atoms being carbon; R³²-quinolyl; R³²-aryl; 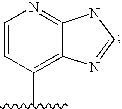
or heterocycloalkyl; wherein the six-membered heteroaryl ring or said five-membered heteroaryl ring is optionally substituted by R⁶;
R³ is H, halogen, C₁-C₆ alkyl, —OH or (C₁-C₆)alkoxy;
R⁴ is independently selected from the group consisting of hydrogen, C₁-C₆ alkyl, C₃-C₆ cycloalkyl, (C₃-C₆)cycloalkyl(C₁-C₆)alkyl, R³³-aryl, R³³-aryl(C₁-C₆)alkyl, and R³²-heteroaryl;
R⁵ is hydrogen, C₁-C₆ alkyl, —C(O)R²⁰, —C(O)₂R²⁰, —C(O)N(R²⁰)₂, R³³-aryl(C₁-C₆)alkyl or (C₁-C₆)alkyl-SO₂—;
R⁶ is 1 to 3 substituents independently selected from the group consisting of —OH, halogen, C₁-C₆ alkyl, C₁-C₆ alkoxy, —CF₃, —NR⁴R⁵, —(C₁-C₆)alkyl-NR⁴R⁵, phenyl, R³³-phenyl, NO₂, —CO₂R⁴, —CON(R⁴)₂, —NHC(O)N(R⁴)₂, R³²-heteroaryl-SO₂—NH—, R³²-aryl-(C₁-C₆)alkyl-NH—, R³²-heteroaryl-(C₁-C₆)alkyl-NH—, R³²-heteroaryl-NH—C(O)—NH—, R³⁷-heterocycloalkyl-N(R²⁹)—C(O)— and R³⁷-heterocycloalkyl-N(R²⁹)—C(O)—NH—;
R¹² is independently selected from the group consisting of C₁-C₆ alkyl, hydroxyl, C₁-C₆ alkoxy, or fluoro, provided that when R¹² is hydroxy or fluoro, then R¹² is not bound to a carbon adjacent to a nitrogen; or R¹² forms a C₁ to C₂ alkyl bridge from one ring carbon to another ring carbon;
R¹³ is independently selected from the group consisting of C₁-C₆ alkyl, hydroxyl, C₁-C₆ alkoxy, or fluoro, provided that when R¹³ is hydroxy or fluoro then R¹³ is not bound to a carbon adjacent to a nitrogen; or forms a C₁ to C₂ alkyl bridge from one ring carbon to another ring carbon; or R¹³ is ═O;
R²⁰ is independently selected from the group consisting of hydrogen, C₁-C₆ alkyl, or aryl, wherein the aryl group is optionally substituted with from 1 to 3 groups independently selected from halogen, —CF₃, —OCF₃, hydroxyl, or methoxy; or when two R²⁰ groups are present, said two R²⁰ groups taken together with the nitrogen to which they are bound can form a five or six membered heterocyclic ring;
R²² is C₁-C₆ alkyl, R³⁴-aryl or heterocycloalkyl;
R²⁴ is H, C₁-C₆ alkyl, —SO₂R²² or R³⁴-aryl;
R²⁵ is independently selected from the group consisting of C₁-C₆ alkyl, halogen, CN, —CF₃, —OH, C₁-C₆ alkoxy, (C₁-C₆)alkyl-C(O)—, aryl-C(O)—, N(R⁴)(R⁵)—C(O)—, N(R⁴)(R⁵)—S(O)₁₋₂—, halo-(C₁-C₆)alkyl- or halo-(C₁-C₆)alkoxy-(C₁-C₆)alkyl-;
R²⁹ is H, C₁-C₆ alkyl, R³⁵-aryl or R³⁵-aryl(C₁-C₆)alkyl-;
R³⁰ is H, C₁-C₆ alkyl-, R³⁵-aryl or R³⁵-aryl(C₁-C₆)alkyl-;
R³¹ is H, C₁-C₆ alkyl-, R³⁵-aryl, R³⁵-aryl(C₁-C₆)alkyl-, (C₁-C₆)alkyl-C(O)—, R³⁵aryl-C(O)—, N(R⁴)(R⁵)—C(O)—, (C₁-C₆)alkyl-S(O)₂— or R³⁵-aryl-S(O)₂—;
or R³⁰ and R³¹together are —(CH₂)₄₋₅—, —(CH₂)₂—O—(CH₂)₂— or —(CH₂)₂—N(R²⁹)—(CH₂)₂— and form a ring with the nitrogen to which they are attached;
R³² is 1 to 3 substituents independently selected from the group consisting of H, —OH, halogen, C₁-C₆ alkyl, C₁-C₆ alkoxy, R³⁵-aryl-O—, —SR²², —CF₃, —OCF₃, —OCHF₂, —NR⁴R⁵, phenyl, R³³-phenyl, —NO₂, —CO₂R⁴, —CON(R⁴)₂, —S(O)₂R²², —S(O)₂N(R²⁰)₂, —N(R²⁴)S(O)₂R²², —CN, hydroxy-(C₁-C₆)alkyl-, —OCH₂CH₂OR²², and R³⁵-aryl(C₁-C₆)-alkyl-O—, wherein the aryl group is optionally substituted with 1 to 3 independently selected halogens;
R³³ is 1 to 3 substituents independently selected from the group consisting of C₁-C₆ alkyl, halogen, —CN, —NO₂, —OCHF₂ and —O—(C₁-C₆)alkyl;
R³⁴ is 1 to 3 substituents independently selected from the group consisting of H, halogen, —CF₃, —OCF₃, —OH and —OCH₃;
R³⁵ is 1 to 3 substituents independently selected from the group consisting of hydrogen, halo, C₁-C₆ alkyl, hydroxy, C₁-C₆ alkoxy, phenoxy, —CF₃, —N(R³⁶)₂, —COOR²⁰ and —NO₂;
R³⁶ is independently selected from the group consisting of H and C₁-C₆ alkyl; and
R³⁷ is independently selected from the group consisting of H, C₁-C₆ alkyl and (C₁-C₆)alkoxycarbonyl; or
(vi) a compound of Formula (VI): 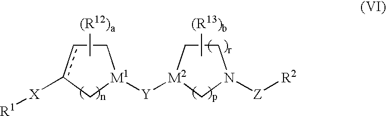
or a pharmaceutically acceptable salt, solvate, ester or prodrug thereof, wherein:
the dotted line represents an optional double bond;
a is 0 to 2;
b is 0 to 2;
n is 1, 2 or3;
p is 1, 2 or3;
r is 0, 1, 2, or 3;
with the provisos that when M² is N, p is not 1; and that when r is 0, M² is C(R³); and that the sum of p and r is 1 to 4;
M¹ is C(R³) or N;
M² is C(R³) or N;
X is a bond or C₁-C₆ alkylene;
Y is —C(O)—, —C(S)—, —(CH₂)_{q}—, —NR⁴C(O)—, —C(O)NR⁴—, —C(O)CH₂—, —SO₂—, —N(R⁴)—, —NH—C(═N—CN)— or —C(═N—CN)—NH—; with the provisos that when M¹ is N, Y is not —NR⁴C(O)— or —NH—C(═N—CN)—; when M² is N, Y is not —C(O)NR⁴— or —C(═N—CN)—NH—; and when Y is —N(R⁴)—, M¹ is CH and M² is C(R³);
q is 1 to 5, provided that when both M¹ and M² are N, q is 2 to 5;
Z is a bond, C₁-C₆ alkylene, C₁-C₆ alkenylene, —C(O)—, —CH(CN)—, —SO₂— or —CH₂C(O)NR⁴—;
R¹ is 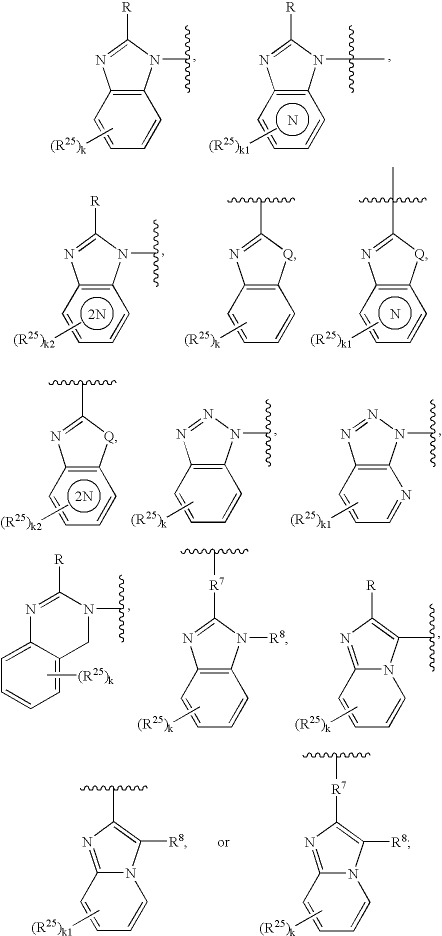
Q is —N(R⁸)—, —S— or —O—;
k is 0, 1, 2, 3 or 4;
k1 is 0, 1, 2 or 3;
k2 is 0, 1 or 2;
R is H, C₁-C₆ alkyl, halo(C₁-C₆)alkyl-, C₁-C₆ alkoxy, (C₁-C₆)alkoxy-(C₁-C₆)alkyl-, (C₁-C₆)-alkoxy-(C₁-C₆)alkoxy, (C₁-C₆)alkoxy-(C₁-C₆)alkyl-SO₀₋₂, R³²-aryl(C₁-C₆)alkoxy-, R³²-aryl(C₁-C₆)alkyl-, R³²-aryl, R³²-aryloxy, R³²-heteroaryl, (C₃-C₆)cycloalkyl, (C₃-C₆)cycloalkyl-(C₁-C₆)alkyl, (C₃-C₆)cycloalkyl-(C₁-C₆)alkoxy, (C₃-C₆)cycloalkyl-oxy-, R³⁷-heterocycloalkyl, R³⁷-heterocycloalkyl-oxy-, R³⁷-heterocycloalkyl-(C₁-C₆)alkoxy, N(R³⁰)(R³¹)−(C₁-C₆)alkyl-, −N(R³⁰)(R³¹), —NH—(C₁-C₆)alkyl-O—(C₁-C₆)alkyl, —NHC(O)NH(R²⁹); R²⁹—S(O)₀₋₂—, halo(C₁-C₆)alkyl-S(O)₀₋₂—, N(R³⁰)(R³¹)—(C₁-C₆)alkyl-S(O)₀₋₂— or benzoyl;
R⁸ is H, C₁-C₆ alkyl, halo(C₁-C₆)alkyl-, (C₁-C₆)alkoxy-(C₁-C₆)alkyl-, R³²-aryl(C₁-C₆)alkyl-, R³²-aryl, R³²-heteroaryl, (C₃-C₆)cycloalkyl, (C₃-C₆)cycloalkyl-(C₁-C₆)alkyl, R³⁷-heterocycloalkyl, N(R³⁰)(R³¹)—(C₁-C₆)alkyl-, R²⁹—S(O)₂—, halo(C₁-C₆)alkyl-S(O)₂—, R²⁹—S(O)₀₋₁—(C₂-C₆)alkyl-, halo(C₁-C₆)alkyl-S(O)₀₋₁—(C₂-C₆)alkyl-;
R² is a six-membered heteroaryl ring having 1 or 2 heteroatoms independently selected from N or N—O, with the remaining ring atoms being carbon; a five-membered heteroaryl ring having 1, 2, 3 or 4 heteroatoms independently selected from N, O or S, with the remaining ring atoms being carbon; R³²-quinolyl; R³²-aryl; heterocycloalkyl; (C₃-C₆)cycloalkyl; C₁-C₆ alkyl; hydrogen; thianaphthenyl; 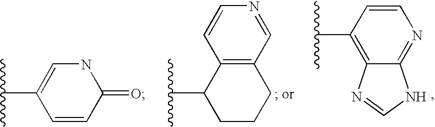
wherein the six-membered heteroaryl ring or said five-membered heteroaryl ring is optionally substituted by R⁶;
R³ is H, halogen, C₁-C₆ alkyl, —OH, (C₁-C₆)alkoxy or —NHSO₂—(C₁-C₆)alkyl;
R⁴ is independently selected from the group consisting of hydrogen, C₁-C₆ alkyl, C₃-C₆ cycloalkyl, (C₃-C₆)cycloalkyl(C₁-C₆)alkyl, R³³-aryl, R³³-aryl(C₁-C₆)alkyl, and R³²-heteroaryl;
R⁵ is hydrogen, C₁-C₆ alkyl, —C(O)R²⁰, —C(O)₂R²⁰, —C(O)N(R²⁰)₂, (C₁-C₆)alkyl-SO₂—, or (C₁-C₆)alkyl-SO₂—NH—;
or R⁴ and R⁵, together with the nitrogen to which they are attached, form an azetidinyl, pyrrolidinyl, piperidinyl, piperazinyl or morpholinyl ring;
R⁶ is 1 to 3 substituents independently selected from the group consisting of —OH, halogen, C₁-C₆ alkyl-, C₁-C₆ alkoxy, C₁-C₆ alkylthio, —CF₃, —NR⁴R⁵, —CH₂—NR⁴R⁵, —NHSO₂R²², —N(SO₂R²²)₂, phenyl, R³³-phenyl, NO₂, —CO₂R⁴, —CON(R⁴)₂, 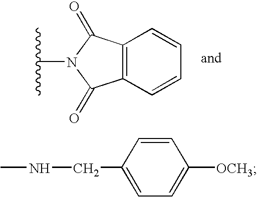
R⁷ is —N(R²⁹)—, —O— or —S(O)₀₋₂—;
R¹² is independently selected from the group consisting of C₁-C₆ alkyl, hydroxyl, C₁-C₆ alkoxy, or fluoro, provided that when R¹² is hydroxy or fluoro, then R¹² is not bound to a carbon adjacent to a nitrogen; or two R¹² substituents form a C₁ to C₂ alkyl bridge from one ring carbon to another non-adjacent ring carbon; or R¹² is ═O;
R¹³ is independently selected from the group consisting of C₁-C₆ alkyl, hydroxyl, C₁-C₆ alkoxy, or fluoro, provided that when R¹³ is hydroxy or fluoro then R¹³ is not bound to a carbon adjacent to a nitrogen; or two R¹³ substituents form a C₁ to C₂ alkyl bridge from one ring carbon to another non-adjacent ring carbon; or R¹³ is ═O;
R²⁰ is independently selected from the group consisting of hydrogen, C₁-C₆ alkyl, or aryl, wherein the aryl group is optionally substituted with from 1 to 3 groups independently selected from halogen, —CF₃, —OCF₃, hydroxyl, or methoxy; or when two R²⁰ groups are present, said two R²⁰ groups taken together with the nitrogen to which they are bound can form a five or six membered heterocyclic ring;
R²² is C₁-C₆ alkyl, R³⁴-aryl or heterocycloalkyl;
R²⁴ is H, C₁-C₆ alkyl, —SO₂R²² or R³⁴-aryl;
R²⁵ is independently selected from the group consisting of C₁-C₆ alkyl, halogen, —CN, —NO₂, —CF₃, —OH, C₁-C₆ alkoxy, (C₁-C₆)alkyl-C(O)—, aryl-C(O)—, —C(O)OR²⁹, —N(R⁴)(R⁵), N(R⁴)(R⁵)—C(O)—, N(R⁴)(R⁵)—S(O)₁₋₂—, R²²—S(O)₀₋₂—, halo-(C₁-C₆)alkyl- or halo-(C₁-C₆)alkoxy-(C₁-C₆)alkyl-;
R²⁹ is H, C₁-C₆ alkyl, C₃-C₆ cycloalkyl, R³⁵-aryl or R³⁵-aryl(C₁-C₆)alkyl-;
R³⁰ is H, C₁-C₆ alkyl-, R³⁵-aryl or R³⁵-aryl(C₁-C₆)alkyl-;
R³¹ is H, C₁-C₆ alkyl-, R³⁵-aryl, R³⁵-aryl(C₁-C₆)alkyl-, R³⁵-heteroaryl, (C₁-C₆)alkyl-C(O)—, R³⁵-aryl-C(O)—, N(R⁴)(R⁵)—C(O)—, (C₁-C₆)alkyl-S(O)₂— or R³⁵-aryl-S(O)₂—;
or R³⁰ and R³¹together are —(CH₂)₄₋₅—, —(CH₂)₂—O—(CH₂)₂— or —(CH₂)₂—N(R³⁸)—(CH₂)₂— and form a ring with the nitrogen to which they are attached;
R³² is 1 to 3 substituents independently selected from the group consisting of H, —OH, halogen, C₁-C₆ alkyl, C₁-C₆ alkoxy, R³⁵-aryl-O—, —SR²², —CF₃, —OCF₃, —OCHF₂, —NR³⁹R⁴⁰, phenyl, R³³-phenyl, NO₂, —CO₂R³⁹, —CON(R³⁹)₂, —S(O)₂R²², —S(O)₂N(R²⁰)₂, —N(R²⁴)S(O)₂R²², —CN, hydroxy-(C₁-C₆)alkyl-, —OCH₂CH₂OR²², and R³⁵-aryl(C₁-C₆)alkyl-O—, or two R³² groups on adjacent carbon atoms together form a —OCH₂O— or —O(CH₂)₂O— group;
R³³ is 1 to 3 substituents independently selected from the group consisting of C₁-C₆ alkyl, halogen, —CN, —NO₂, —CF₃, —OCF₃, —OCHF₂ and —O—(C₁-C₆)alkyl;
R³⁴ is 1 to 3 substituents independently selected from the group consisting of H, halogen, —CF₃, —OCF₃, —OH and —OCH₃;
R³⁵ is 1 to 3 substituents independently selected from hydrogen, halo, C₁-C₆ alkyl, hydroxy, C₁-C₆ alkoxy, phenoxy, —CF₃, —N(R³⁶)₂, —COOR²⁰ and —NO₂;
R³⁶ is independently selected form the group consisting of H and C₁-C₆ alkyl;
R³⁷ is 1 to 3 substituents independently selected from hydrogen, halo, C₁-C₆ alkyl, hydroxy, C₁-C₆ alkoxy, phenoxy, —CF₃, —N(R³⁶)₂, —COOR²⁰, —C(O)N(R²⁹)₂ and —NO₂, or R³⁷ is one or two ═O groups;
R³⁸ is H, C₁-C₆ alkyl, R³⁵-aryl, R³⁵-aryl(C₁-C₆)alkyl-, (C₁-C₆)alkyl-SO₂ or halo(C₁-C₆)alkyl-SO₂—;
R³⁹ is independently selected from the group consisting of hydrogen, C₁-C₆ alkyl, C₃-C₆ cycloalkyl, (C₃-C₆)cycloalkyl(C₁-C₆)alkyl, R³³-aryl, R³³-aryl(C₁-C₆)alkyl, and R³²-heteroaryl; and
R⁴⁰ is hydrogen, C₁-C₆ alkyl, —C(O)R²⁰, —C(O)₂R²⁰, —C(O)N(R²⁰)₂, (C₁-C₆)alkyl-SO₂—, or (C₁-C₆)alkyl-SO₂—NH—;
or R³⁹ and R⁴⁰, together with the nitrogen to which they are attached, form an azetidinyl, pyrrolidinyl, piperidinyl, piperazinyl or morpholinyl ring; or
(vii) a compound of Formula (VII): 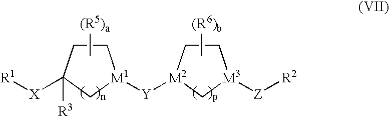
or a pharmaceutically acceptable salt, solvate, prodrug or ester thereof, wherein:
a is 0, 1 or 2;
b is 0, 1 or2;
n is 1, 2 or 3;
p is 1, 2 or 3;
M¹ is CH or N;
M² is CH, CF or N;
M³ is CH or N
with the proviso that when M² and M³ are each N, p is 2 or 3;
Y is —C(═O)—, —C(═S)—, —(CH₂)_{q}—, —C(═NOR⁷)— or —SO₁₋₂—;
q is 1, 2, 3, 4 or 5, provided that when M¹ and M² are both N, q is 2, 3, 4 or 5;
X is —N(R⁴)—, —N(R⁴)—CH(R¹⁹)—, —CH(R¹⁹)—N(R⁴)—, —(CH₂)ᵣ—C(O)—N(R⁴)—, —O—(CH₂)₂—C(O)—N(R⁴)—, —CH₂—O—(CH₂)₃—C(O)—N(R⁴)—, —(CH₂)ₜ—N(R⁴)—C(O)—, —C(O)—N(R⁴)—CH₂—, —(CH₂)ᵣ—N(R¹⁹)C(O)N(R¹⁹)—, —N(R¹⁹)C(O)N(R¹⁹)—(CH₂)ᵣ—, —(CH₂)ₜ—OC(O)N(R¹⁹)—, —N(R¹⁹)C(O)O—, —O—, —OCH₂—, —CH₂O—, —OC(O)—, —C(O)O—, —S—, —S(O)— or —SO₂—;
r is 0, 1, 2 or 3;
t is 0 or 1;
Z is a bond, R⁸-alkylene, —CH(R²⁰)—CH(R²⁰)—O—, —CH(R²⁰)—CH(R²⁰)—N—, —CH(R²⁰)—(R²³—C₁-C₅ alkylene), —CH(R²⁰)—C(R²⁰)═C(R²⁰)—, —CH(R²⁰)—C(R²⁰)═C(R²⁰)—R²³—C₁-C₃ alkylene) or R⁸-alkylene interrupted by a cycloalkylene or heterocycloalkylene group, provided that when M³ is N and Z is R⁸-alkylene interrupted by a heterocycloalkylene group bonded through a ring nitrogen, the alkylene portion of the Z group has 2-4 carbon atoms between M³ and said nitrogen;
R¹ is H, R¹⁰-alkyl, R¹⁰-cycloalkyl, R¹⁰-aryl, R¹⁰-heteroaryl or R¹⁰-heterocycloalkyl;
R² is R¹⁶-alkyl, R¹⁶-alkenyl, R¹⁶-aryl, R¹⁶-heteroaryl, R¹⁶-cycloalkyl or R¹⁶-heterocycloalkyl;
R³ is H, alkyl, R²¹-aryl, R²²-cycloalkyl, R²²-heterocycloalkyl, R²¹-heteroaryl or —C(O)NH₂;
R⁴ is H, alkyl, haloalkyl, R¹⁸-aryl, R¹⁸-heteroaryl, R¹⁸-arylalkyl, —C(O)R¹² or —SO₂R¹³;
R⁵ and R⁶ are each independently selected from the group consisting of halo, alkyl, —OH, alkoxy, —CF₃ and —CN; or two R⁵ substituents on the same carbon atom or two R⁶ substituents on the same carbon atom form ═O;
R⁷ is H, alkyl, haloalkyl, aryl or heteroaryl;
R⁸ is 1, 2 or 3 substituents independently selected from the group consisting of H, cycloalkyl, heterocycloalkyl, aryl, heteroaryl and —CF₃;
each R⁹ is independently selected from the group consisting of H and alkyl;
R¹⁰ is 1, 2, 3 or 4 substituents independently selected from the group consisting of H, halo, alkyl, —OH, alkoxy, aryl, arylalkyl, heteroaryl, heteroarylalkyl, aryloxy, —CF₃, —OCF₃, —NO₂, —C(O)-alkyl, —C(O)-heterocycloalkyl, —CO₂R¹¹, —N(R¹¹)₂, —CON(R¹¹)₂, —NHC(O)R¹¹, —NHC(O)-alkoxyalkyl-, —NHC(O)—CH₂—NHC(O)CH₃, —NHSO₂R¹¹, —CH(═NOR¹⁹), —SO₂N(R¹¹)₂, —SO₂CF₃ and —CN;
each R¹¹ is independently selected from the group consisting of H, alkyl, haloalkyl, R¹⁸-aryl, R¹⁸-heteroaryl, R¹⁸-arylalkyl, cycloalkyl and heterocycloalkyl;
R¹² is alkyl, cycloalkyl, aryl, heteroaryl or heterocycloalkyl;
R¹³ is alkyl, aryl or alkylsulfonylalkyl;
R¹⁶ is 1, 2 or 3 substituents independently selected from the group consisting of H, halo, alkyl, —OH, alkoxy, hydroxyalkyl, aryl, aryloxy, —CF₃, —OCF₃, —NO₂, —CO₂R¹⁷, —N(R¹⁷)₂, -alkylene-N(R¹⁷)₂, —CON(R¹⁷)₂, —NHC(O)R¹⁷, —NHC(O)OR¹⁷, —NHSO₂R¹⁷, —SO₂N(R¹⁷)₂ and —CN;
each R¹⁷ is independently selected from the group consisting of H, alkyl, haloalkyl, aryl, heteroaryl, cycloalkyl and heterocycloalkyl;
R¹⁸ is 1, 2 or 3 substituents independently selected from the group consisting of H, alkyl, halo, alkoxy, —CF₃ and -alkylene-N(R¹⁷)₂;
R¹⁹ is independently selected from the group consisting of H and alkyl;
R²⁰ is independently selected from the group consisting of H and alkyl;
R²¹ is 1, 2, 3 or 4 substituents independently selected from the group consisting of H, halo, alkyl, —OH, alkoxy, —CF₃, —CHF₂, —OCF₃, —NO₂, —CN, —C(O)N(R¹⁹)₂ and —N(R¹⁹)₂;
R²² is 1, 2 or 3 substituents independently selected from the group consisting of halo, alkyl, —OH, alkoxy, —CF₃ and —CN; and
R²³ is 1, 2 or 3 substituents independently selected from the group consisting of H, alkyl, cycloalkyl, heterocycloalkyl, aryl, heteroaryl, —CF₃, halo, —CN, —OH, alkoxy, —OCF₃, —NO₂, and —N(R⁹)₂; or
(viii) a compound of Formula (VIII): 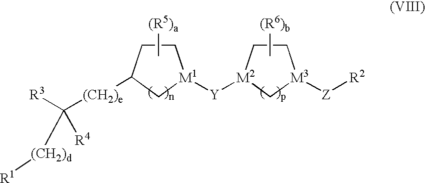
or a pharmaceutically acceptable salt, solvate, ester or prodrug thereof, wherein:
a is 0, 1 or 2;
b is 0, 1 or 2;
d is 0 or 1;
e is 0 or 1
n is 1, 2 or 3;
p is 1, 2 or 3;
M¹ is CH or N;
M² is CH, CF or N;
M³ is CH or N
with the proviso that when M² and M³ are each N, p is 2 or 3;
Y is —C(═O)—, —C(═S)—, —(CH₂)_{q}—, —C(═NOR⁷)— or —SO₁₋₂—;
q is 1 to 5, provided that when M¹ and M² are both N, q is 2 to 5;
Z is a bond, R⁸-alkylene, —CH(R²⁰)—CH(R²⁰)—O—, —CH(R²⁰)—CH(R²⁰)—N—, —CH(R²⁰)—(R²³—C₁-C₅ alkylene), —CH(R²⁰)—C(R²⁰)═C(R²⁰)—, —CH(R²⁰)—C(R²⁰)═C(R²⁰)—(R²³—C₁-C₃ alkylene) or R⁸-alkylene interrupted by a cycloalkylene or heterocycloalkylene group, provided that when M³ is N and Z is R⁸-alkylene interrupted by a heterocycloalkylene group bonded through a ring nitrogen, the alkylene portion of the Z group has 2-4 carbon atoms between M³ and said nitrogen;
R¹ is H, alkyl, alkenyl, R¹⁰-cycloalkyl, R¹⁰-aryl, R¹⁰-pyridyl, R¹⁰-quinolyl or R¹⁰-heterocycloalkyl;
R³ and R⁴ are independently selected from the group consisting of H, halo, alkyl, haloalkyl, hydroxyalkyl, alkoxyalkyl, hydroxyalkoxy, alkoxyalkoxy, aryl, arylalkyl, cycloalkyl, heterocycloalkyl, heteroaryl, heteroarylalkyl, —OR¹², —CN, —(CH₂)_{f}—N(R¹²)₂, —(CH₂)_{f}—N(R¹⁹)—SO₂R¹², —(CH₂)_{f}—N(R¹⁹)—C(O)R¹², —(CH₂)_{f}—NHC(O)NHR¹², —(CH₂)_{f}—NHC(O)OR¹², —O—C(O)NHR¹², —(CH₂)_{f}—C(O)OR¹² and —O—(CH₂)_{f}—C(O)OR¹², provided that when one of R³ and R⁴ is a heteroatom-linked substituent, the other is H;
f is 0, 1 or 2;
or R³ and R⁴, together with the carbon to which they are attached, form —C(═C(R¹⁵)(R¹⁸)—, a 3-7 membered cycloalkyl ring substituted by R¹³, a 3-7-membered heterocycloalkyl ring substituted by R¹³, a R¹³-phenyl ring, or a 5-6-membered heteroaryl ring substituted by R¹³; or when d is 1, or e is 1, or both d and e are 1, R³ and R⁴, together with the carbon to which they are attached, form —C(O)—;
or R¹—(CH₂)_{d}—C(R³)(R⁴)—(CH₂)ₑ— forms 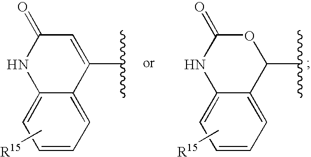
R² is R¹⁶-alkyl, R¹⁶-alkenyl, R¹⁶-aryl, R¹⁶-heteroaryl, R¹⁶-cycloalkyl or R¹⁶-heterocycloalkyl;
R⁵ and R⁶ are each independently selected from the group consisting of halo, alkyl, —OH, alkoxy, —CF₃ and —CN; or two R⁵ substituents on the same carbon atom form ═O;
R⁷ is H, alkyl, haloalkyl, aryl or heteroaryl;
R⁸ is 1, 2 or 3 substituents independently selected from the group consisting of H, cycloalkyl, heterocycloalkyl, aryl, heteroaryl and —CF₃;
each R⁹ is independently selected from the group consisting of H and alkyl;
R¹⁰ is 1 to 4 substituents independently selected from the group consisting of H, halo, alkyl, —OH, alkoxy, aryl, heteroaryl, aryloxy, —CF₃, —CHF₂, —OCF₃, —NO₂, —CO₂R¹¹, —N(R¹¹)₂, —CON(R¹¹)₂, —NHC(O)R¹¹, —NHC(O)OR¹¹, —NHSO₂R¹¹, —SO₂N(R¹¹)₂ and —CN;
each R¹¹ is independently selected from the group consisting of H, alkyl, haloalkyl, aryl, heteroaryl, arylalkyl, cycloalkyl and heterocycloalkyl;
each R¹² is independently selected from the group consisting of H, alkyl, alkenyl, haloalkyl, aryl, arylalkyl, heteroaryl, heteroarylalkyl, cycloalkyl, cycloalkylalkyl and heterocycloalkyl;
R¹³ is 1 to 4 substituents independently selected form the group consisting of H, halo, alkyl, —OH, alkoxy, hydroxyalkyl, alkoxyalkyl, —CO₂R¹⁴, —C(O)N(R¹⁴)₂, —CF₃, and —CN; or two R¹³ substituents on the same carbon atom form ═O;
each R¹⁴ is independently selected from the group consisting of H and alkyl;
R¹⁵ is H, alkyl, halo, aryl or —CF₃;
R¹⁶ is 1 to 3 substituents independently selected from the group consisting of H, halo, alkyl, —OH, alkoxy, aryl, aryloxy, —CF₃, —OCF₃, —NO₂, —CO₂R¹⁷, —N(R¹⁷)₂, —CON(R¹⁷)₂, —NHC(O)R¹⁷, —NHC(O)OR¹⁷, —NHSO₂R¹⁷, —SO₂N(R¹⁷)₂ and —CN;
each R¹⁷ is independently selected from the group consisting of H, alkyl, haloalkyl, aryl, heteroaryl, cycloalkyl and heterocycloalkyl;
R¹⁸ is H, alkyl, halo, aryl, —CF₃, alkoxy, heteroaryl, —O—C(O)R¹², —C(O)N(R¹²)₂, —C(O)OR¹² or —C(O)-heterocycloalkyl;
R¹⁹ is H alkyl or pyridylmethyl;
R²⁰ is independently selected from the group consisting of H and alkyl; and
R²¹ is 1, 2 or 3 substituents independently selected from the group consisting of H, cycloalkyl, heterocycloalkyl, aryl, heteroaryl, —CF₃, halo, —CN, —OH, alkoxy, —OCF₃, —NO₂, and —N(R⁹)₂.

2. The composition of claim 1, wherein the CB₁ antagonist is rimonabant.

3. The composition of claim 1, wherein the H₃ antagonist/inverse agonist is a compound of Formula (I).

4. The composition of claim 1, wherein the H₃ antagonist/inverse agonist is a compound of Formula (II).

5. The composition of claim 1, wherein the H₃ antagonist/inverse agonist is a compound of Formula (III).

6. The composition of claim 1, wherein the H₃ antagonist/inverse agonist is a compound of Formula (IV).

7. The composition of claim 1, wherein the H₃ antagonist/inverse agonist is a compound of Formula (V).

8. The composition of claim 3, wherein the H₃ antagonist/inverse agonist is a compound selected from the group consisting of:

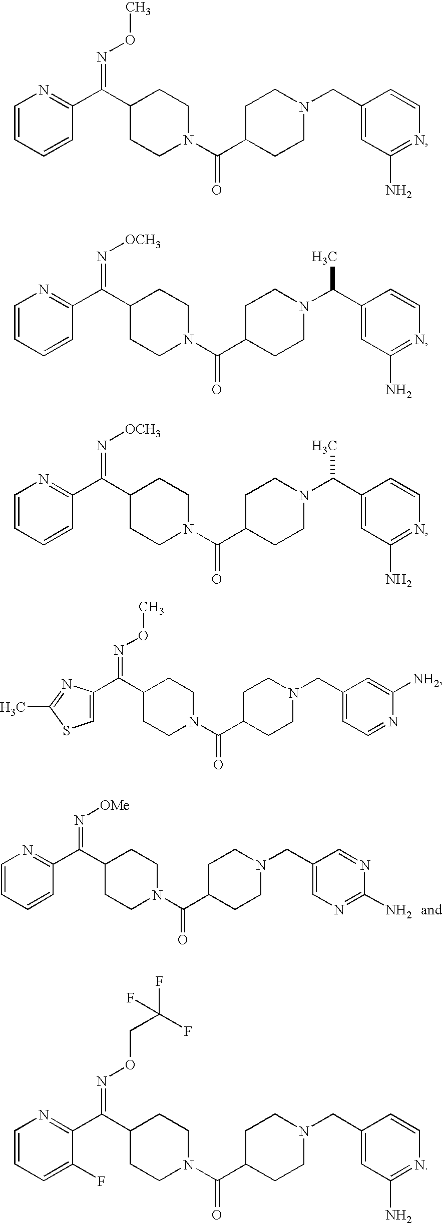

9. The composition of claim 4, wherein the H₃ antagonist/inverse agonist is a compound selected from the group consisting of:

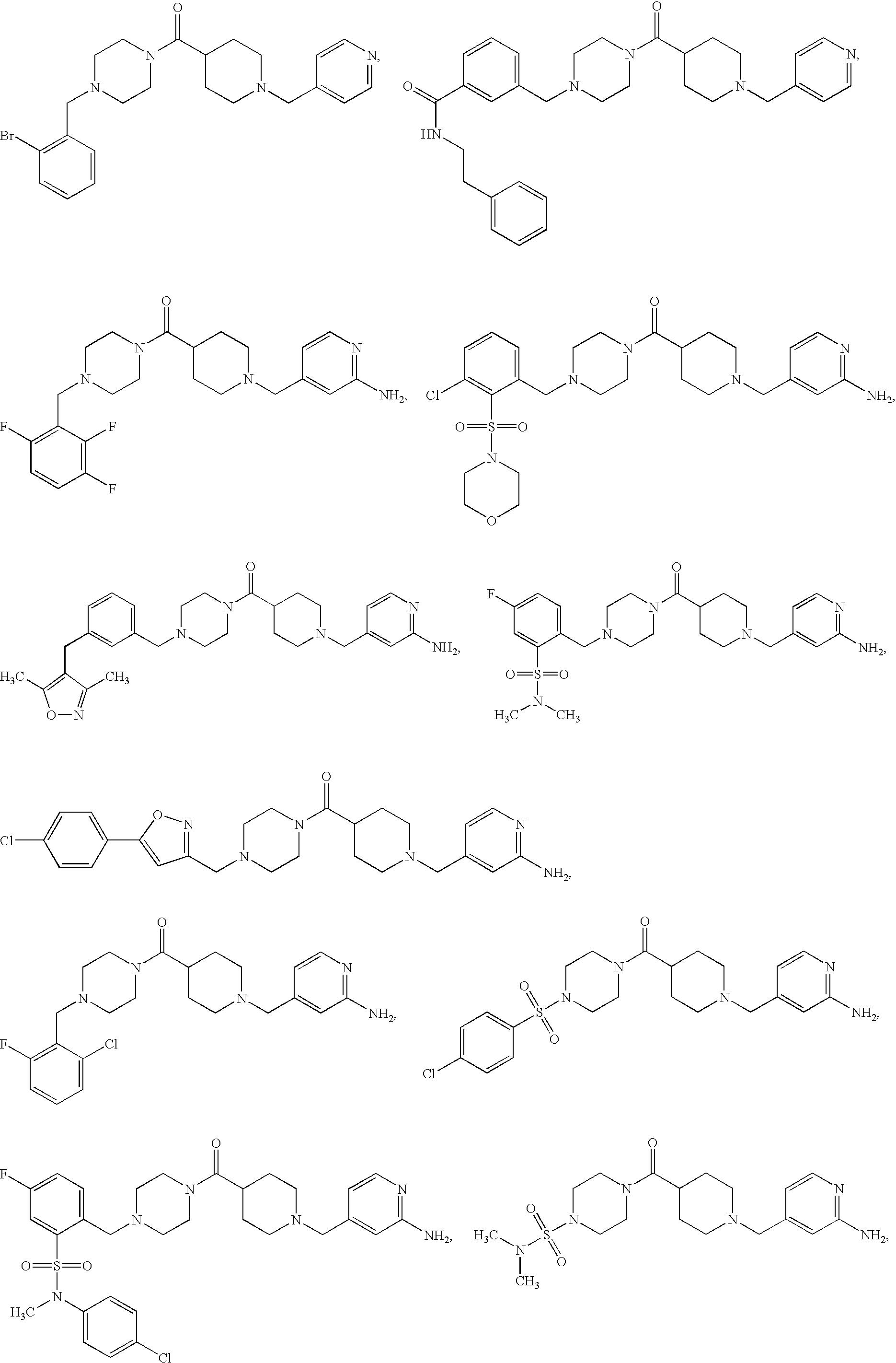

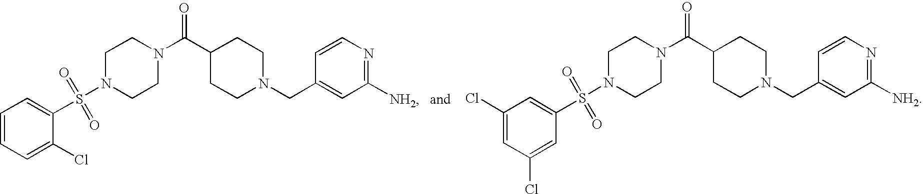

10. The composition of claim 5, wherein the H₃ antagonist/inverse agonist is a compound selected from the group consisting of:

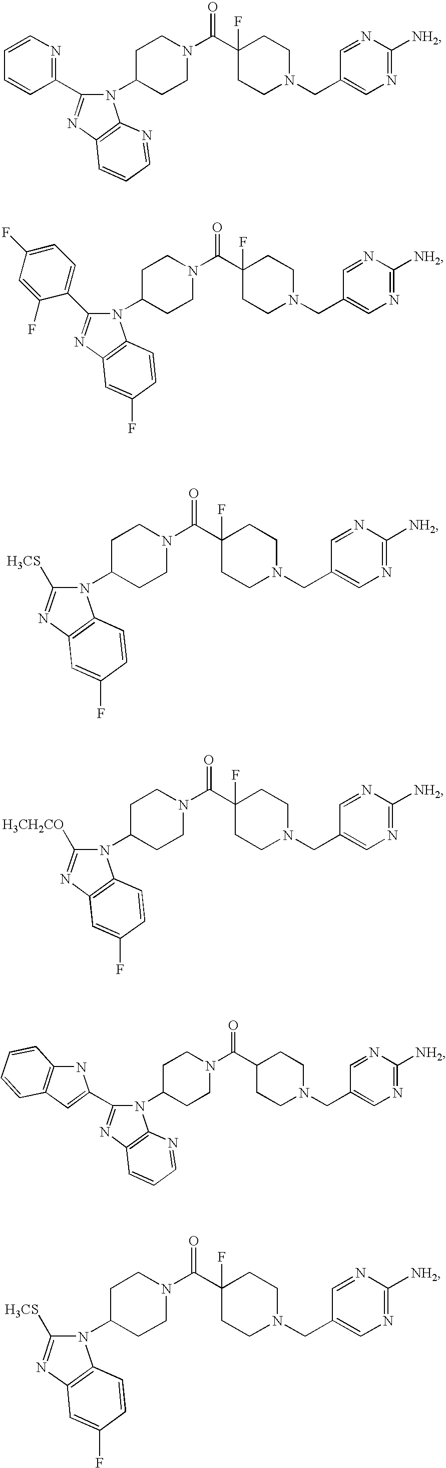

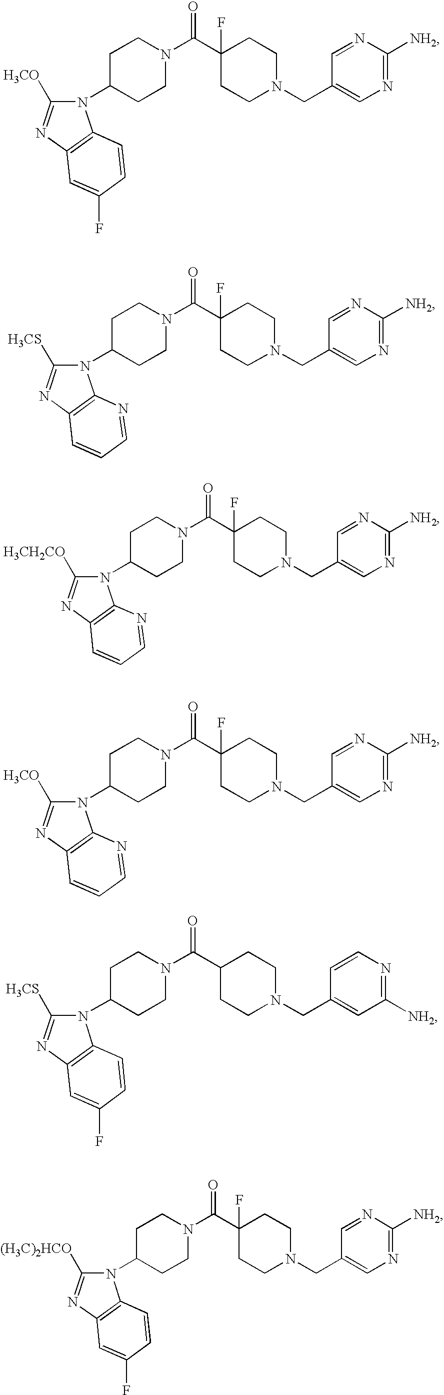

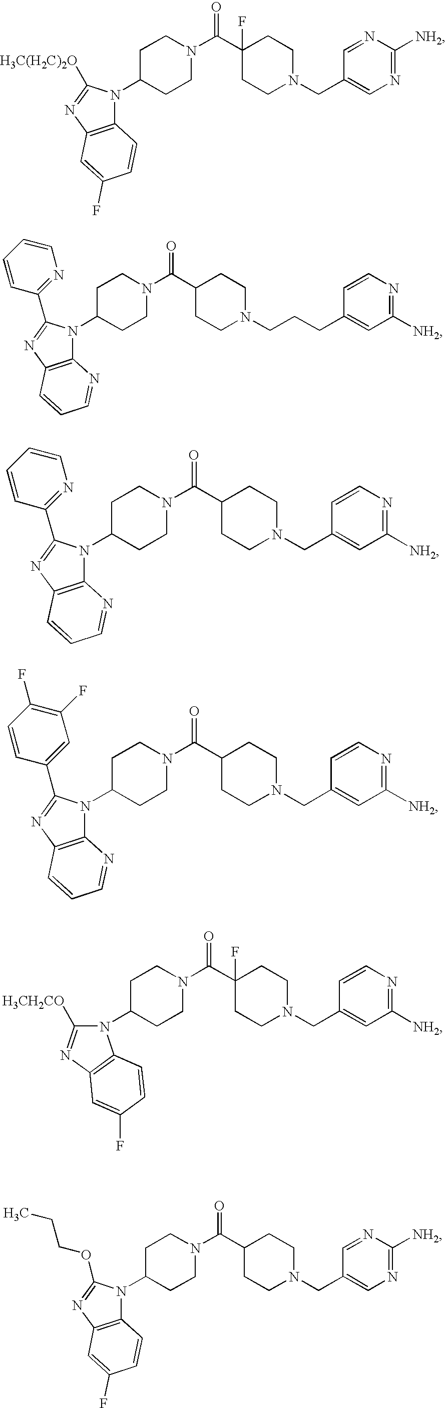

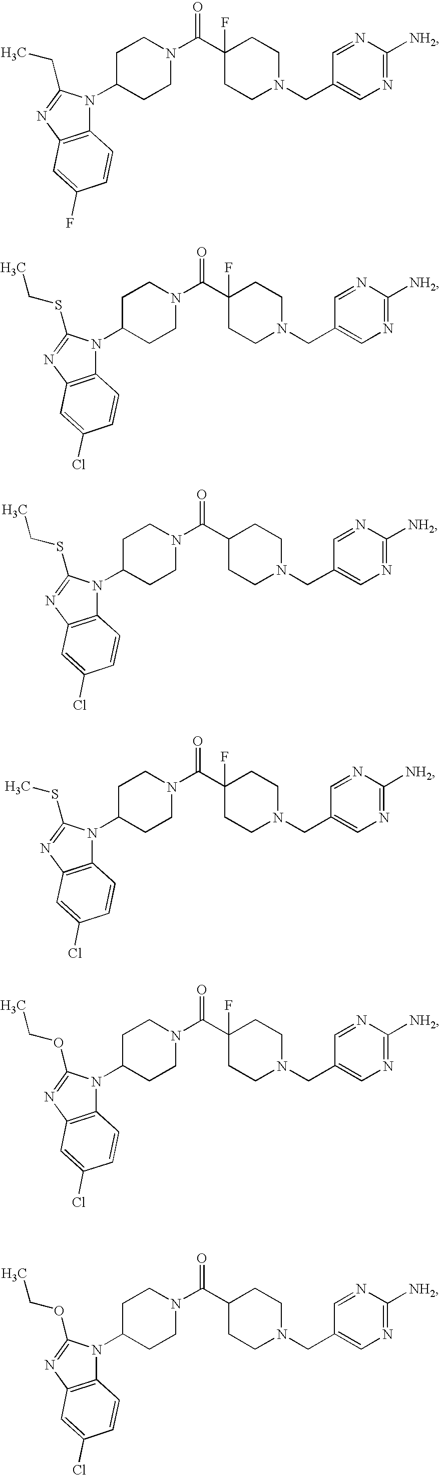

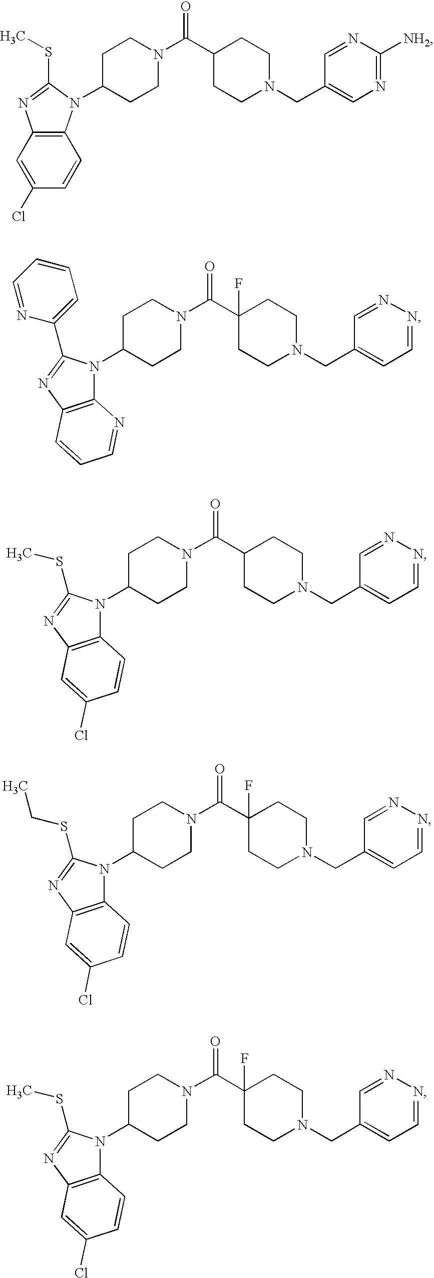

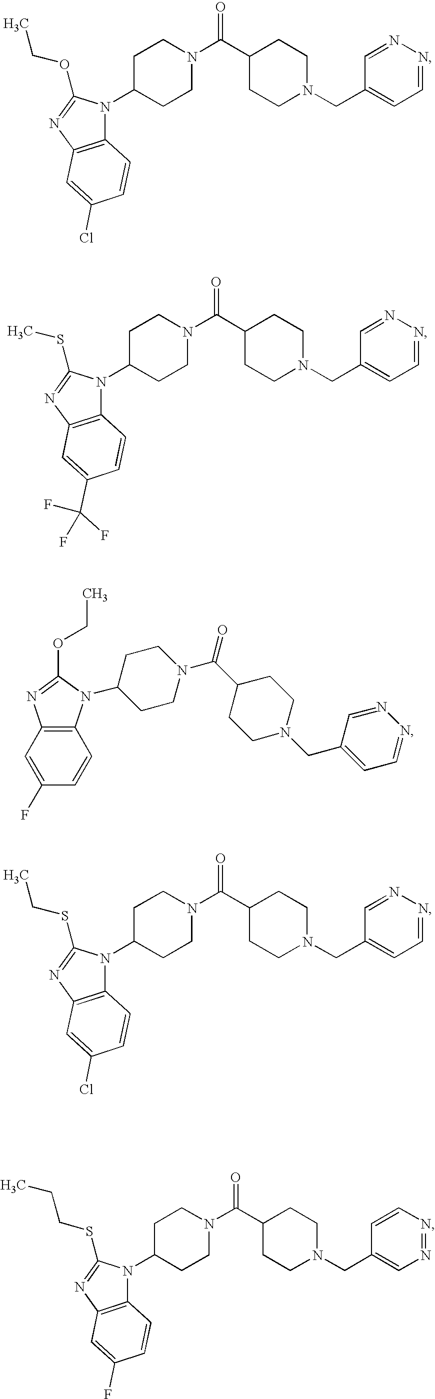

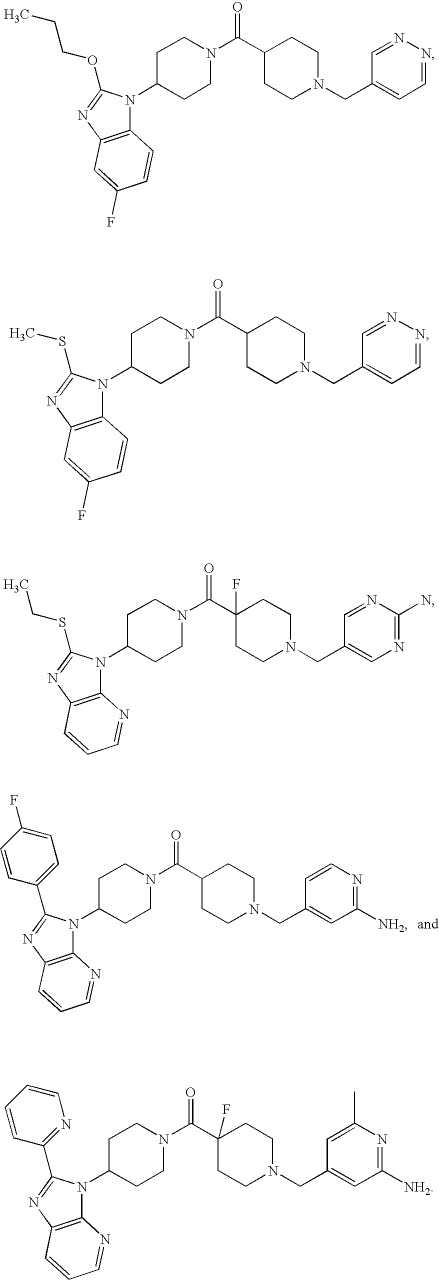

11. The composition of claim 6, wherein the H₃ antagonist/inverse agonist is a compound selected from the group consisting of:

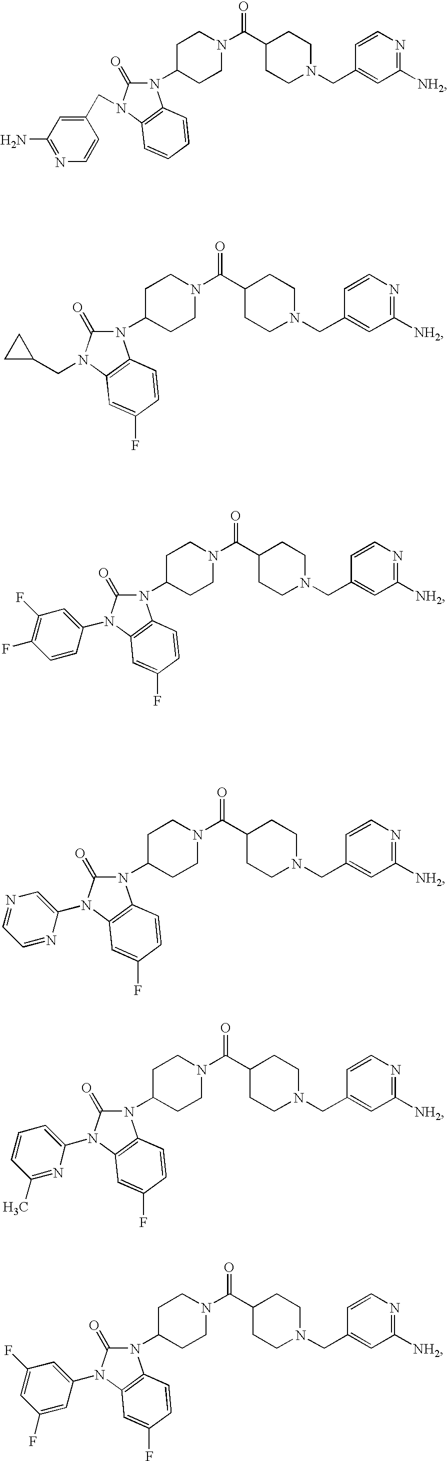

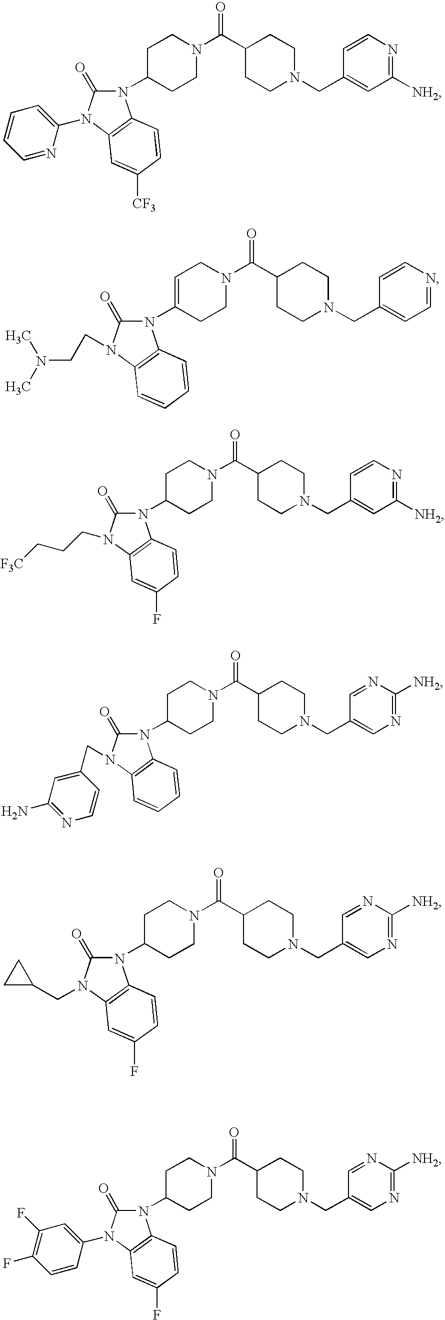

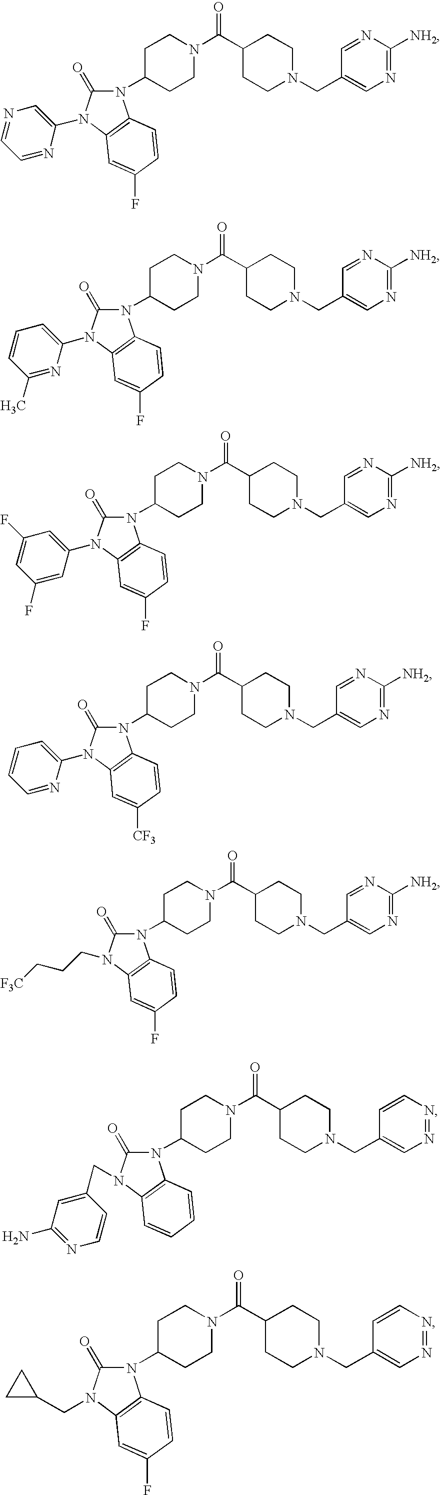

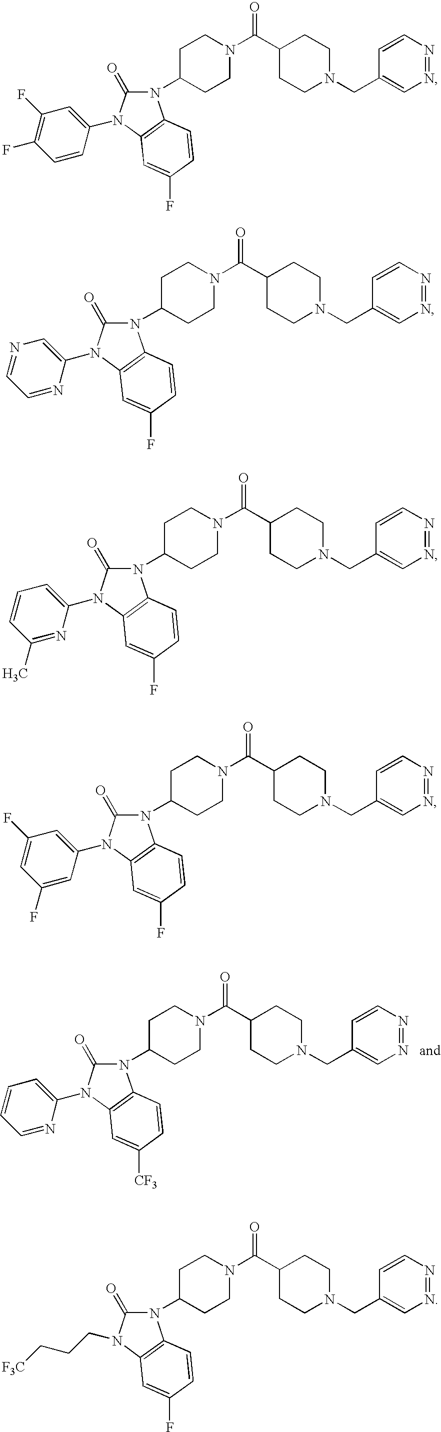

12. The composition of claim 7, wherein the H₃ antagonist/inverse agonist is a compound selected from the group consisting of:

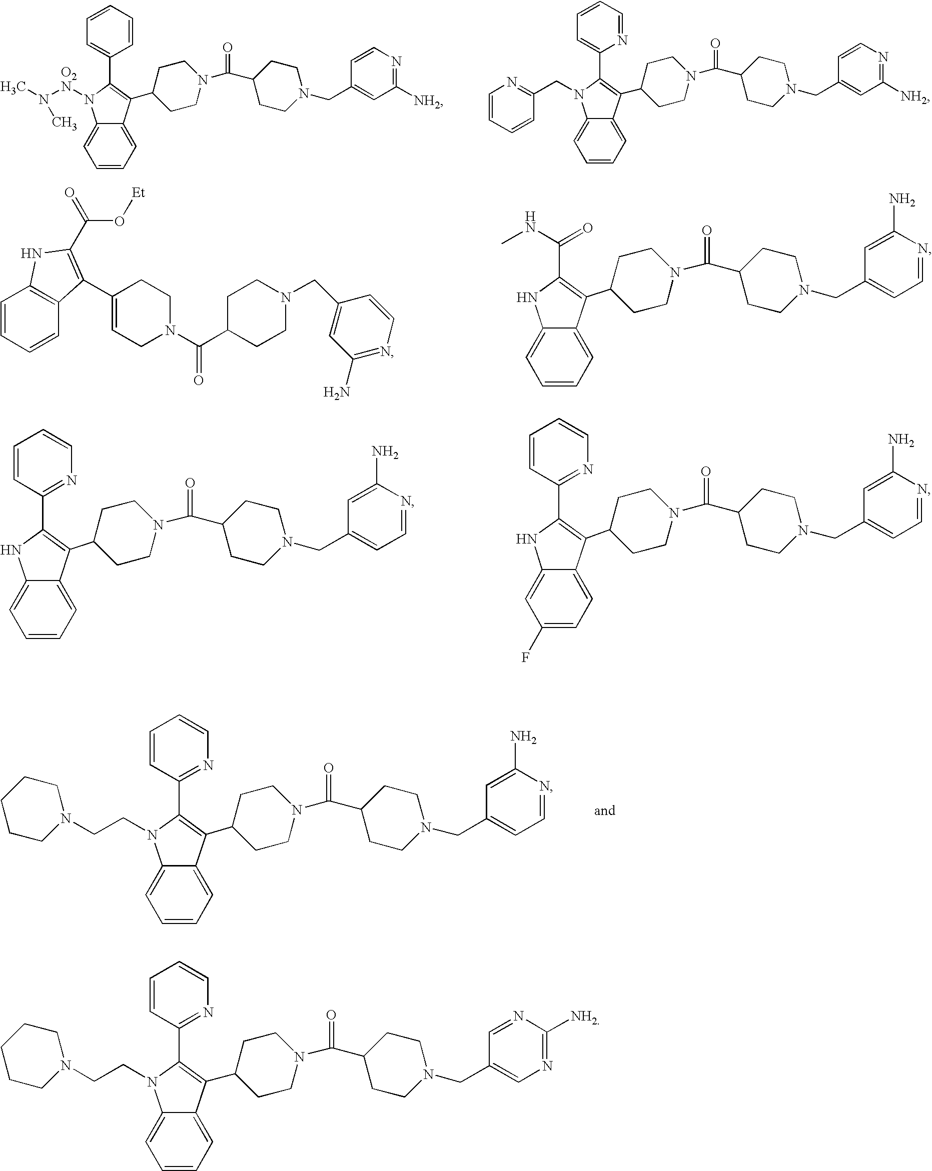

13. The composition of claim 1, wherein the H₃ antagonist/inverse agonist is a compound selected from the group consisting of:

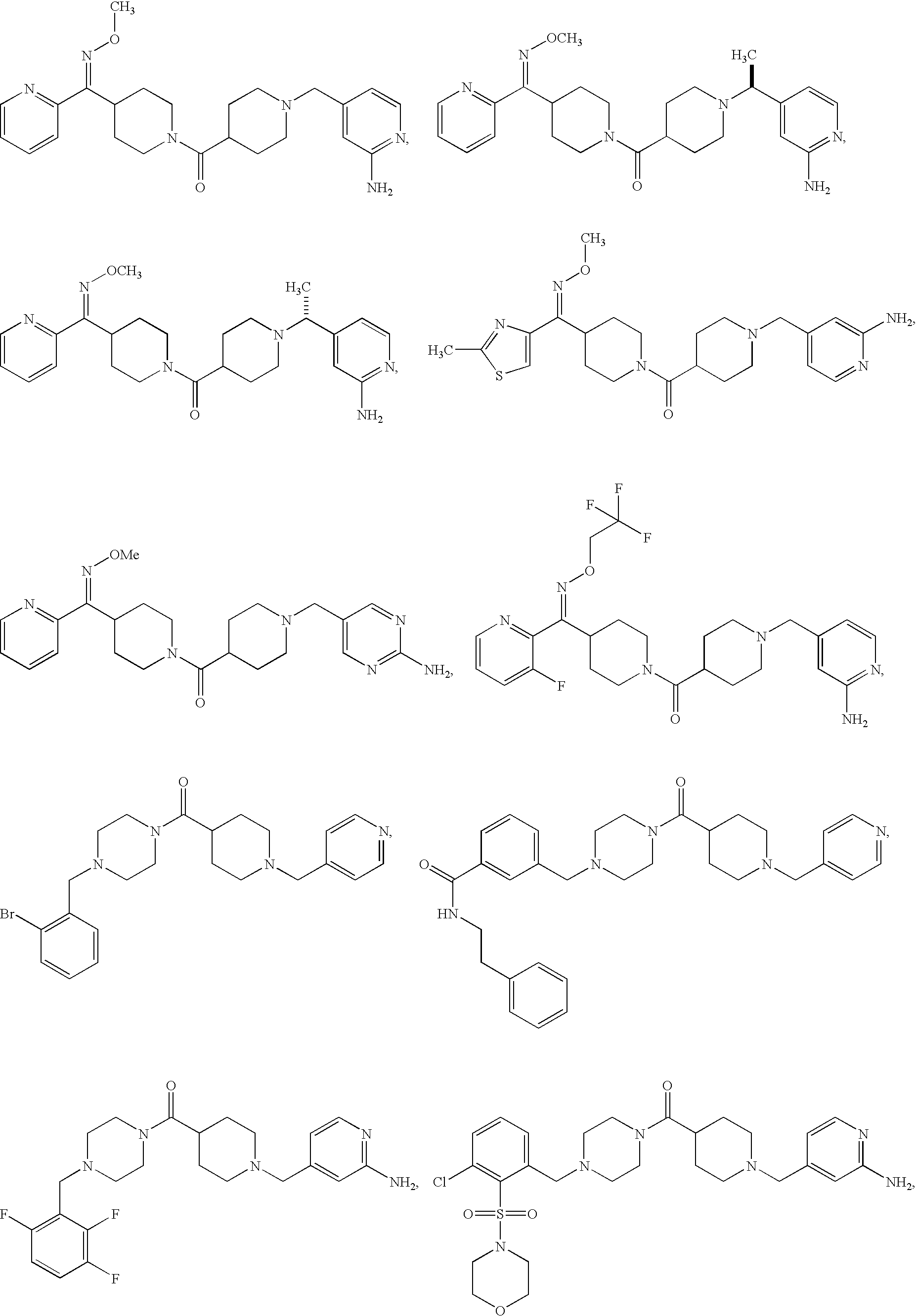

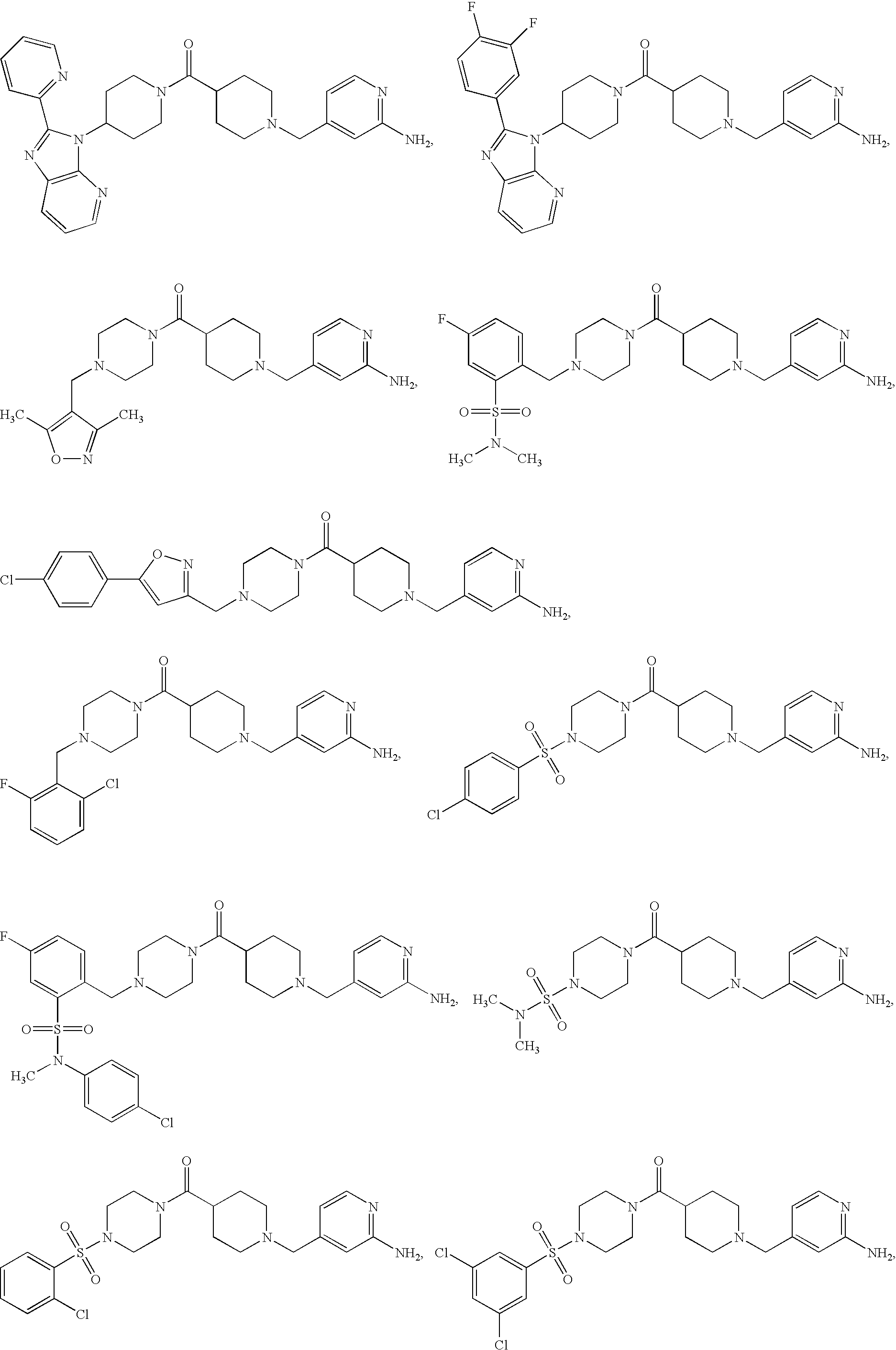

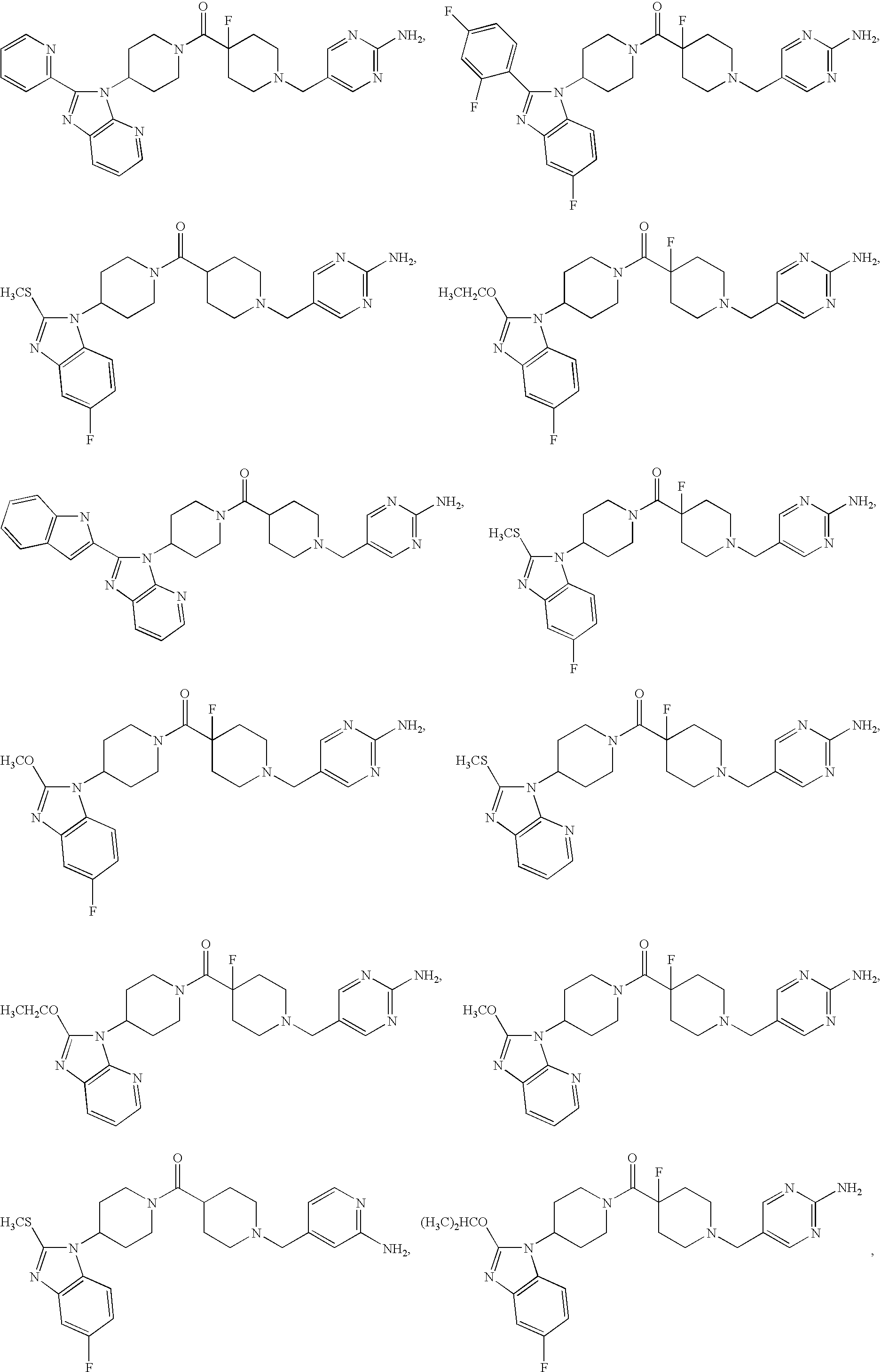

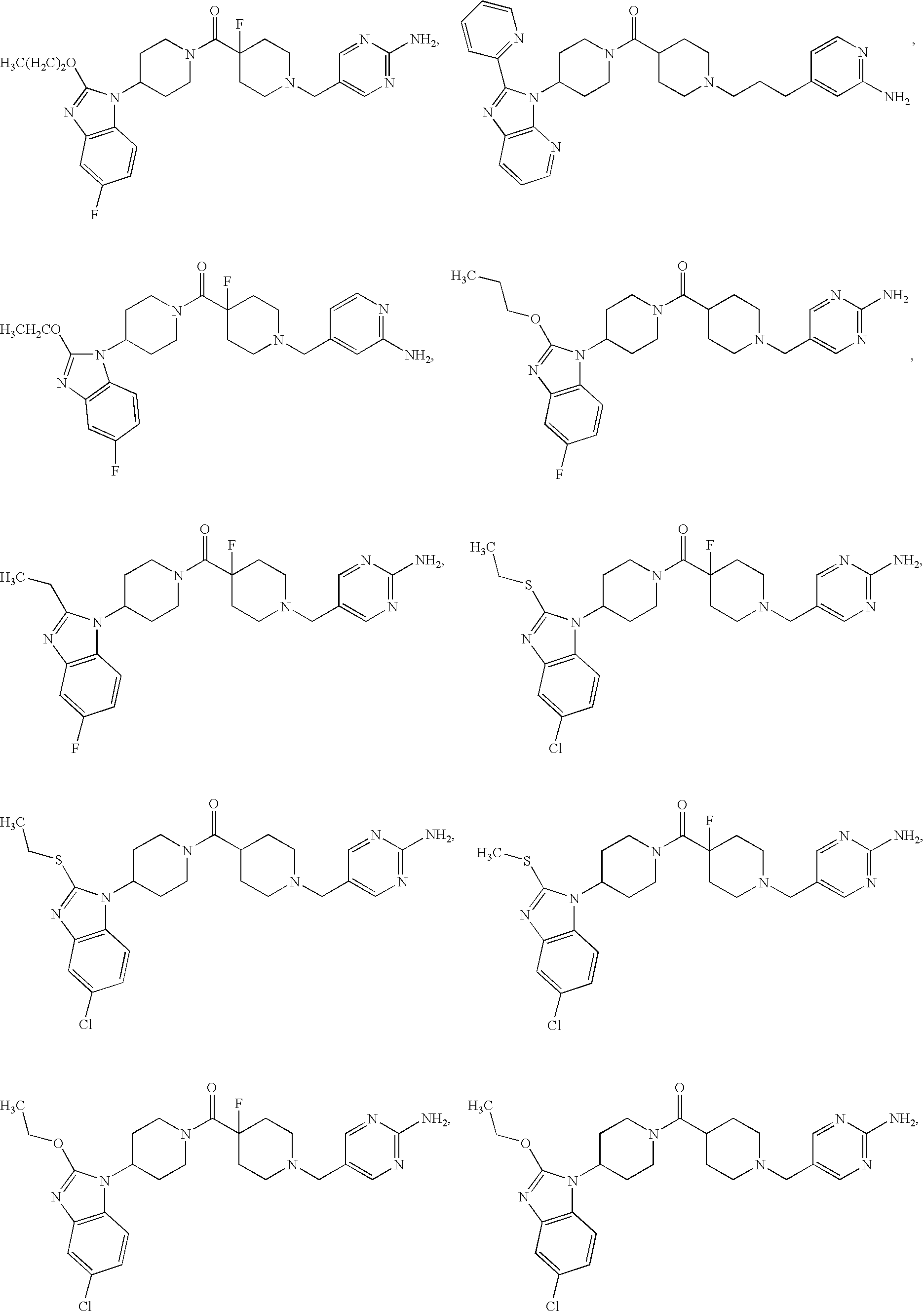

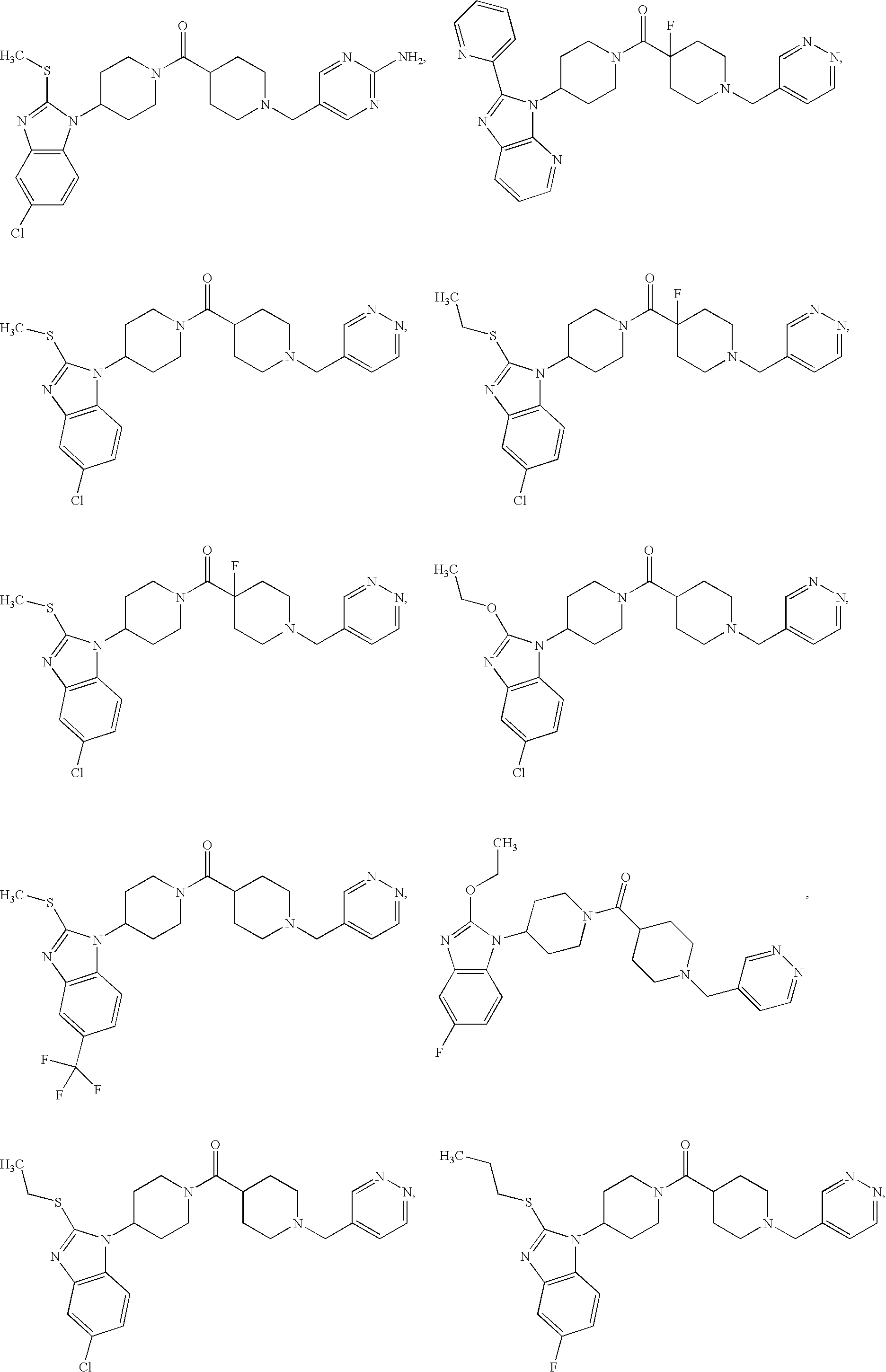

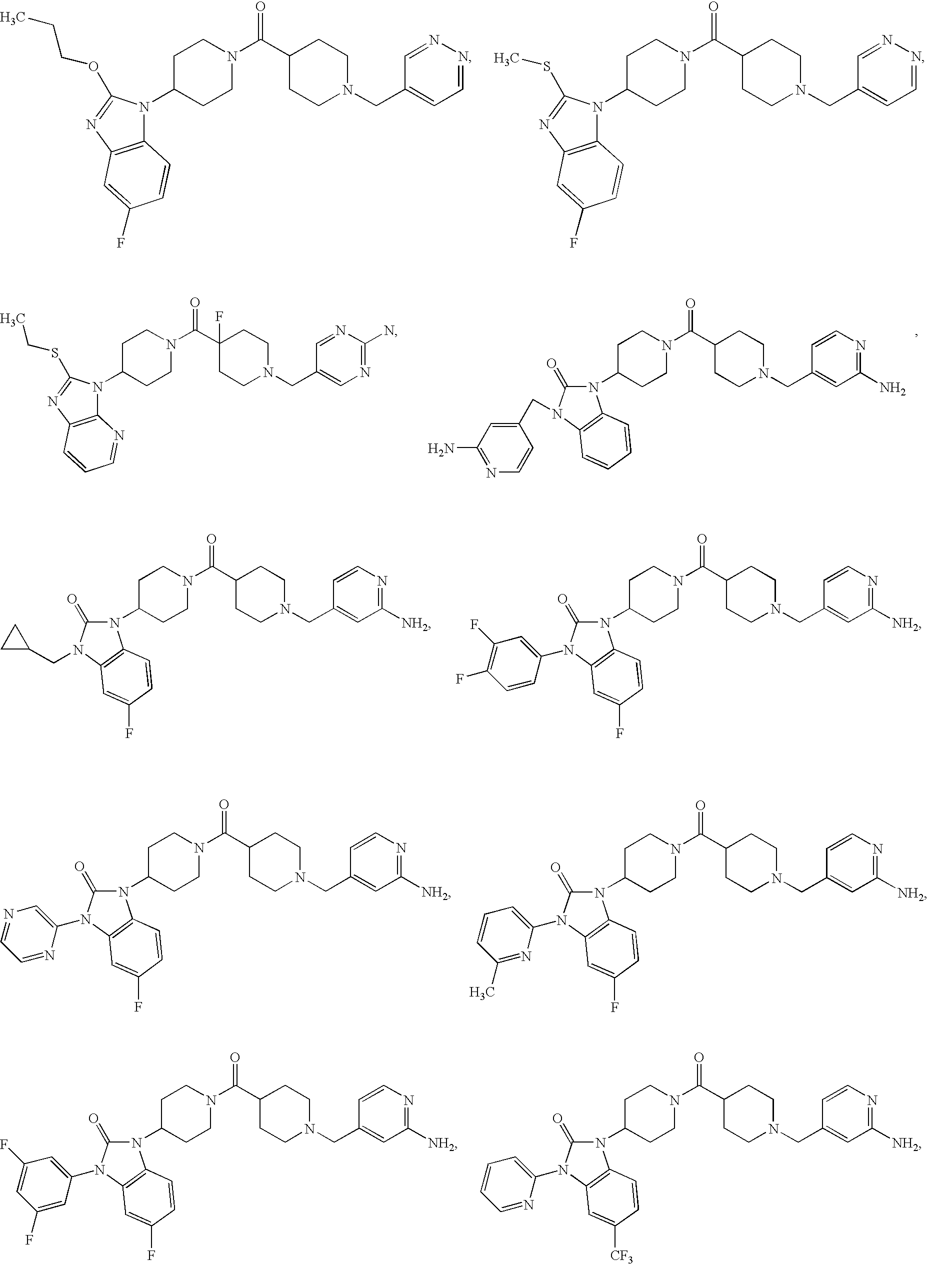

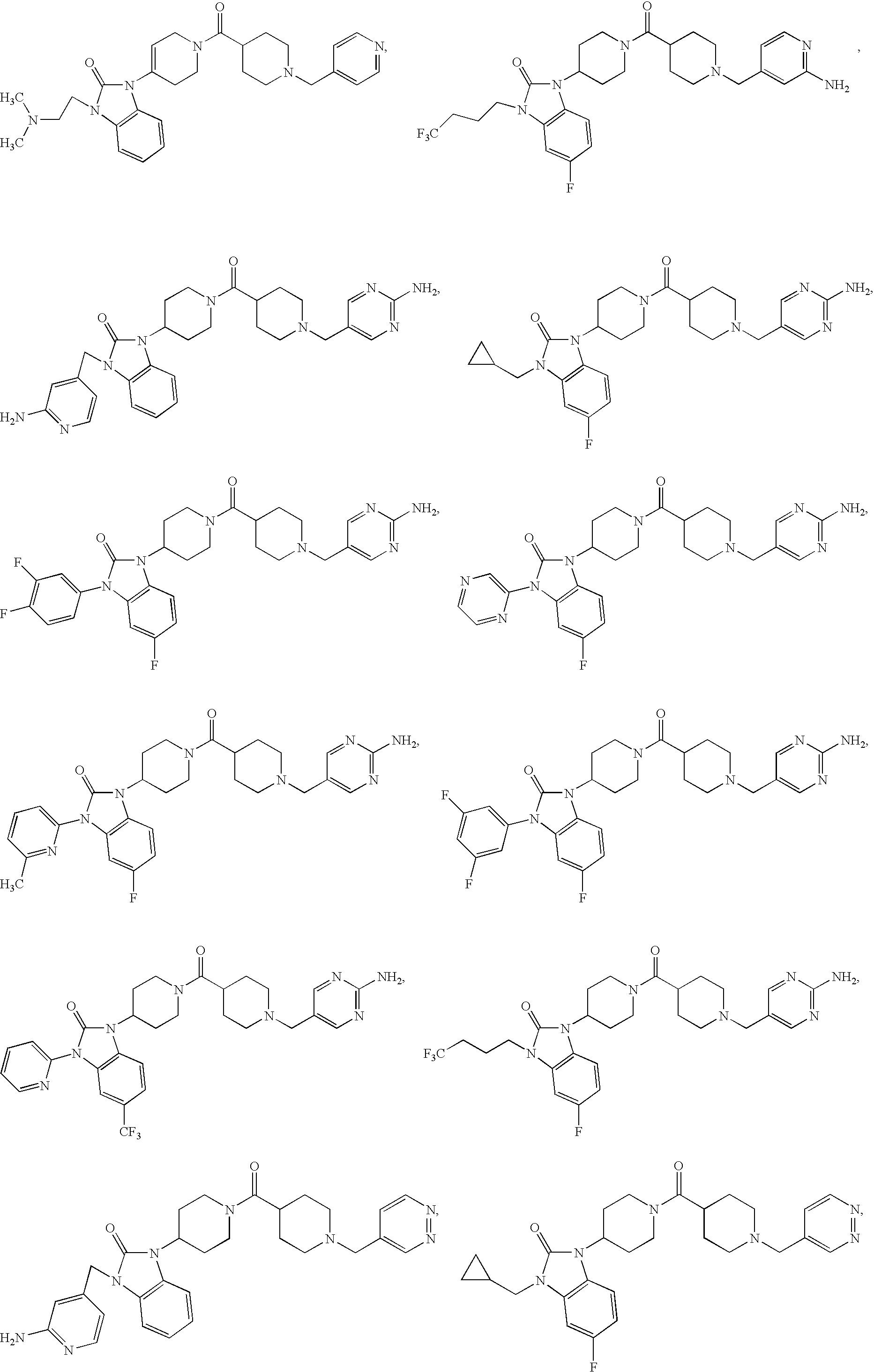

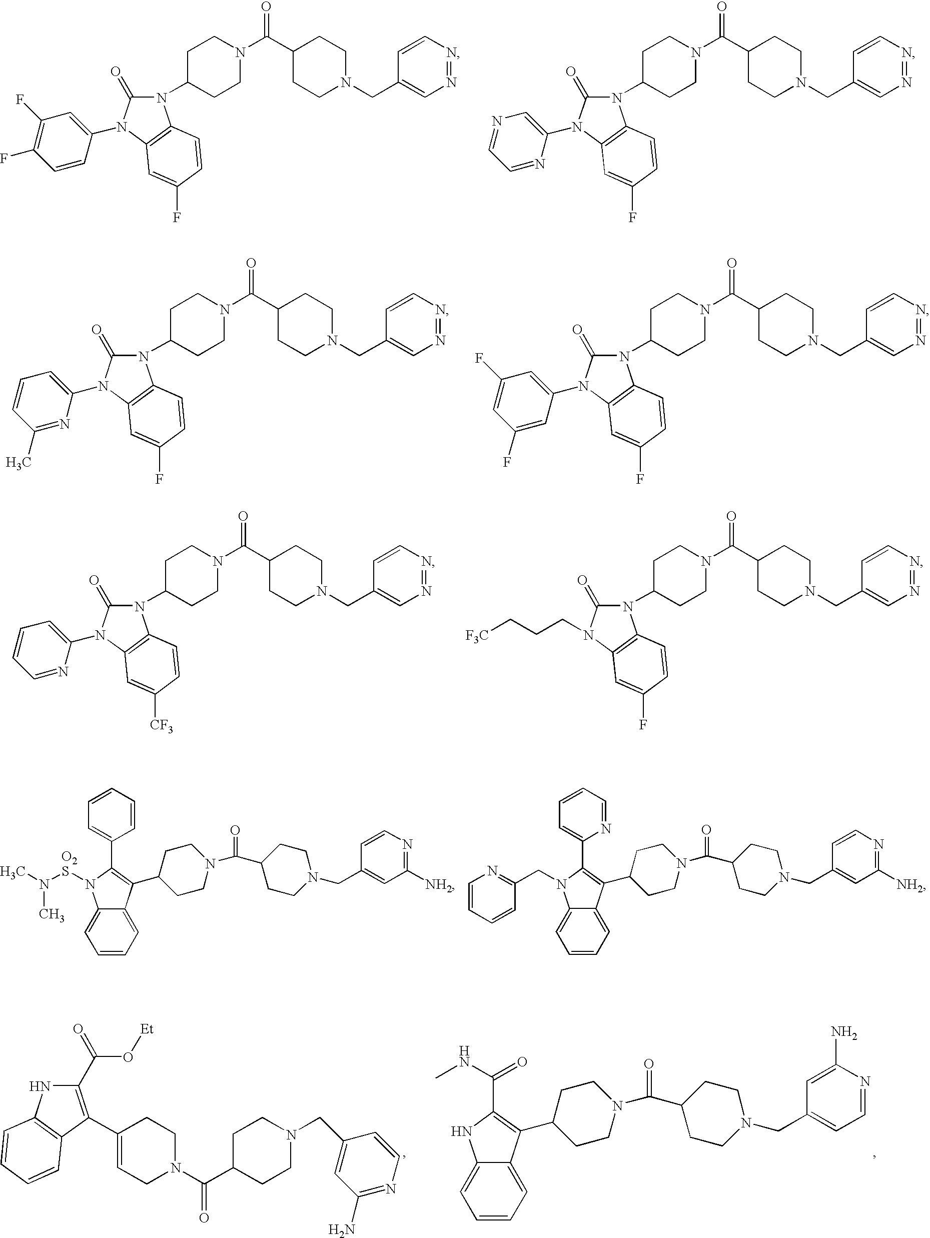

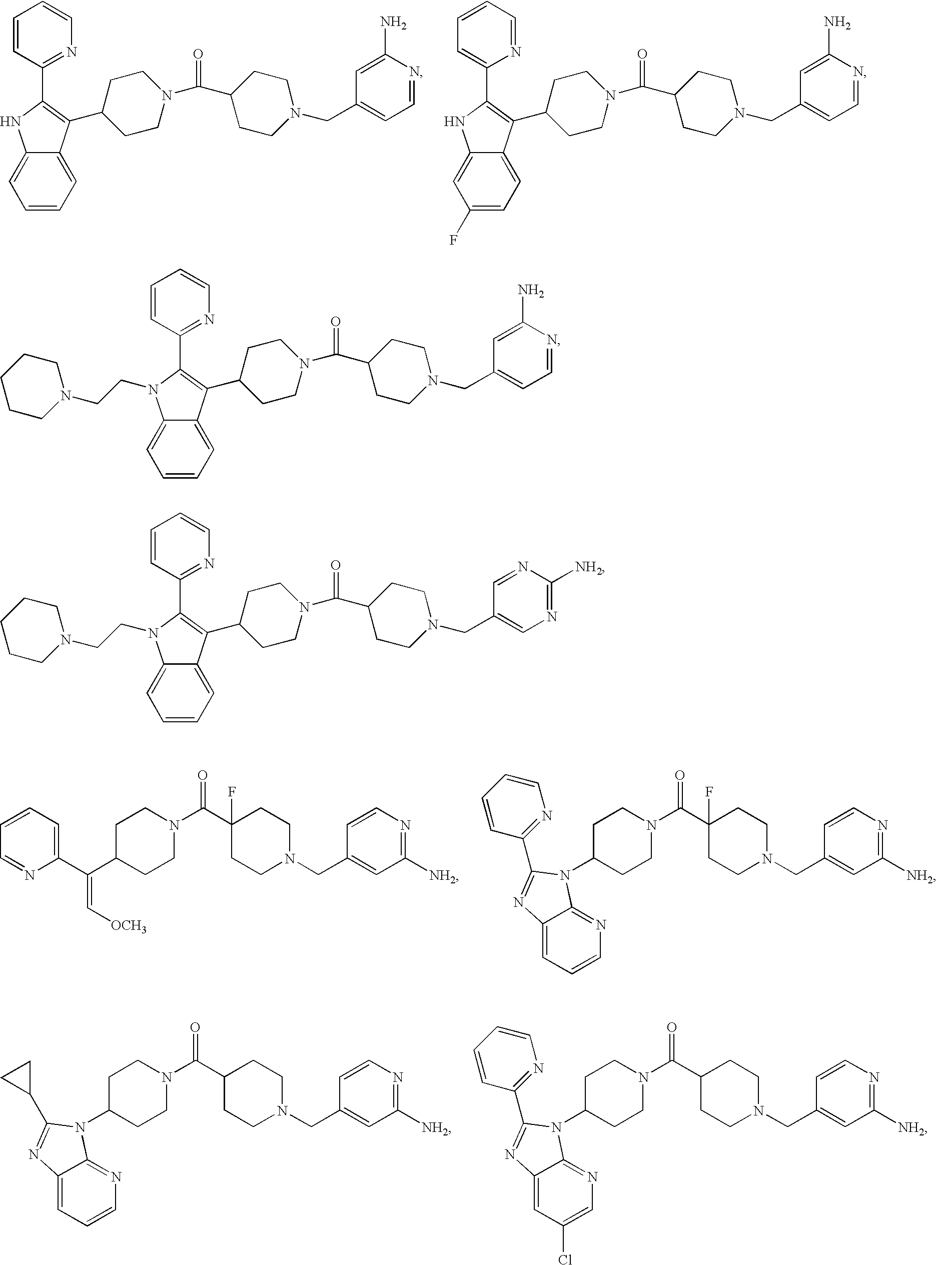

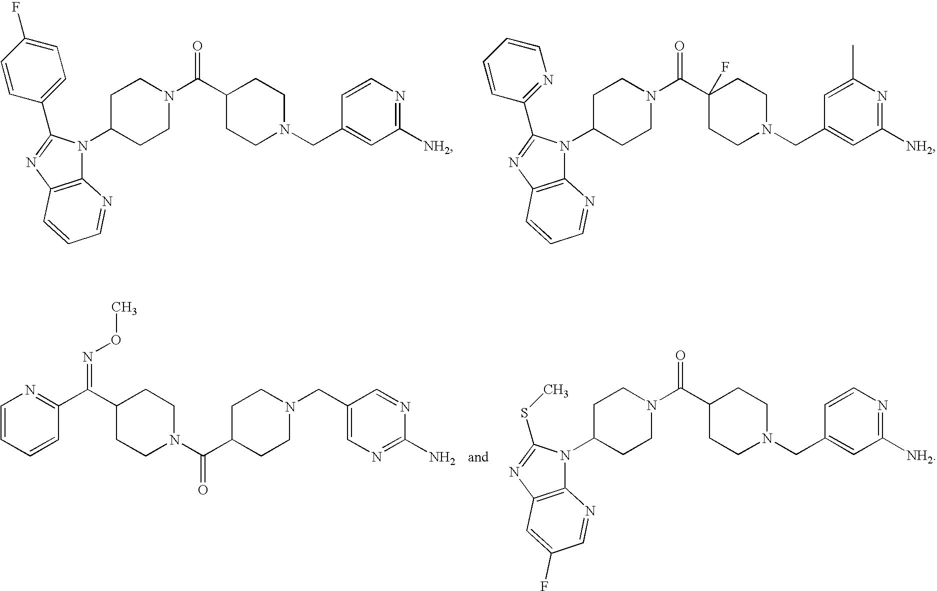

14. The composition of claim 13, wherein the appetite suppressant is rimonabant.

15. The composition of claim 13, wherein the appetite suppressant is phentermine.

16. The composition of claim 13, wherein the appetite suppressant is sibutramine.

17. The composition of claim 13, wherein the appetite suppressant is topiramate.

18. The composition of claim 1, further comprising an HMG-CoA reductase inhibitor.

19. The composition of claim 18, wherein the HMG-CoA reductase inhibitor is pravastatin, lovastatin, simvastatin, fluvastatin, atorvastatin, and rosuvastatin.

20. The composition of claim 19, wherein the HMG-CoA reductase inhibitor is simvastatin.

21. The composition of claim 13, further comprising an HMG-CoA reductase inhibitor.

22. The composition of claim 21, wherein the HMG-CoA reductase inhibitor is pravastatin, lovastatin, simvastatin, fluvastatin, atorvastatin, or rosuvastatin.

23. The composition of claim 22, wherein the HMG-CoA reductase inhibitor is simvastatin.

24. The composition of claim 22, wherein the appetite suppressant is rimonabant.

25. The composition of claim 22, wherein the appetite suppressant is phentermine.

26. The composition of claim 22, wherein the appetite suppressant is sibutramine.

27. The composition of claim 22, wherein the appetite suppressant is topiramate.

28. The composition of claim 1, further comprising an anti-diabetic agent.

29. The composition of claim 13, further comprising an anti-diabetic agent.

30. The composition of claim 22, further comprising an anti-diabetic agent.

31. The composition of claim 28, wherein the anti-diabetic agent is a sulfonylurea, an insulin sensitizer, an α-glucosidase inhibitor, an insulin secretagogue, an anti-obesity agent, a meglitinide, insulin or an insulin-containing composition.

32. The composition of claim 31, wherein the anti-diabetic agent is an insulin sensitizer or a sulfonylurea.

33. The composition of claim 32, wherein the insulin sensitizer is a PPAR activator.

34. The composition of claim 33, wherein the PPAR activator is a thiazolidinedione.

35. The composition of claim 29, wherein the anti-diabetic agent is a sulfonylurea, an insulin sensitizer, an α-glucosidase inhibitor, an insulin secretagogue, an anti-obesity agent, a meglitinide, insulin or an insulin-containing composition.

36. The composition of claim 35, wherein the anti-diabetic agent is an insulin sensitizer or a sulfonylurea.

37. The composition of claim 36, wherein the insulin sensitizer is a PPAR activator.

38. The composition of claim 37, wherein the PPAR activator is a thiazolidinedione.

39. The composition of claim 30, wherein the anti-diabetic agent is a sulfonylurea, an insulin sensitizer, an α-glucosidase inhibitor, an insulin secretagogue, an anti-obesity agent, a meglitinide, insulin or an insulin-containing composition.

40. The composition of claim 39, wherein the anti-diabetic agent is an insulin sensitizer or a sulfonylurea.

41. The composition of claim 40, wherein the insulin sensitizer is a PPAR activator.

42. The composition of claim 41, wherein the PPAR activator is a thiazolidinedione.

43. A method of treating obesity or an obesity-related disorder in a patient comprising administering a therapeutically effective amount of the composition of claim 1 to a patient in need thereof.

44. A method of treating obesity or an obesity-related disorder in a patient comprising administering a therapeutically effective amount of the composition of claim 13 to a patient in need thereof.

45. A method of treating obesity or an obesity-related disorder in a patient comprising administering a therapeutically effective amount of the composition of claim 22 to a patient in need thereof.

46. A method of treating obesity or an obesity-related disorder in a patient comprising administering a therapeutically effective amount of the composition of claim 28 to a patient in need thereof.

47. A method of treating obesity or an obesity-related disorder in a patient comprising administering a therapeutically effective amount of the composition of claim 29 to a patient in need thereof.

48. A method of treating obesity or an obesity-related disorder in a patient comprising administering a therapeutically effective amount of the composition of claim 30 to a patient in need thereof.

49. A method of treating diabetes in a patient comprising administering a therapeutically effective amount of the composition of claim 1 to a patient in need thereof.

50. A method of treating diabetes in a patient comprising administering a therapeutically effective amount of the composition of claim 13 to a patient in need thereof.

51. A method of treating diabetes in a patient comprising administering a therapeutically effective amount of the composition of claim 22 to a patient in need thereof.

52. A method of treating obesity or an obesity-related disorder in a patient comprising administering a therapeutically effective amount of the composition of claim 30 to a patient in need thereof.

53. A method of treating diabetes in a patient comprising administering a therapeutically effective amount of the composition of claim 28 to a patient in need thereof.

54. A method of treating diabetes in a patient comprising administering a therapeutically effective amount of the composition of claim 29 to a patient in need thereof.

55. A method of treating diabetes in a patient comprising administering a therapeutically effective amount of the composition of claim 30 to a patient in need thereof.
